(19) United States
(12) Patent Application Publication
(10) Pub. No.: US 2025/0360201 A1
(43) Pub. Date: Nov. 27, 2025

(54) SARS-COV-2 VARIANTS OF CONCERN-SPECIFIC MULTI-ANTIGENS UNIVERSAL VACCINE

(71) Applicant: THE REGENTS OF THE UNIVERSITY OF CALIFORNIA, Oakland, CA (US)

(72) Inventor: Lbachir BenMohamed, Irvine, CA (US)

(21) Appl. No.: 18/872,496

(22) PCT Filed: Jun. 7, 2023
(86) PCT No.: PCT/US23/68093

§ 371 Date: Dec. 6, 2024

Related U.S. Application Data
(63) Continuation-in-part of application No. 18/046,862, filed on Oct. 14, 2022.
Application No. 18/046,862 is a continuation-in-part of application No. 18/046,875, filed on Oct. 14, 2022.
Application No. 18/046,875 is a continuation-in-part of application No. 18/046,462, filed on Oct. 13, 2022.
(60) Provisional application No. 63/349,904, filed on Jun. 7, 2022.
(60) Provisional application No. 63/349,799, filed on Jun. 7, 2022.
(60) Provisional application No. 63/451,302, filed on Mar. 10, 2023.

(51) Int. Cl. A61K 39/215; A61P 37/04

(52) U.S. Cl. CPC A61K 39/215; A61P 37/04

(57) ABSTRACT

Pan-coronavirus vaccines for inducing efficient, powerful and long-lasting protection against all Coronaviruses infections and diseases, comprising multiple highly conserved large sequences which may comprise one or more conserved B, CD4 and CDS T cell epitopes that help provide multiple targets for the body to develop an immune response for preventing a Coronavirus infection and/or disease. In certain embodiments, the large sequences are conserved proteins or large sequences, e.g., sequences that are highly conserved among human coronaviruses and/or animal coronaviruses (e.g., coronaviruses isolated from animals susceptible to coronavirus infections).

### CROSS-REFERENCES TO RELATED APPLICATIONS

This application claims benefit of U.S. Provisional Application No. 63/349,904 filed Jun. 7, 2022, U.S. Provisional Application No. 63/349,799 filed Jun. 7, 2022, and U.S. Provisional Application No. 63/451,302 filed Mar. 10, 2023, the specifications of which are incorporated herein in their entirety by reference.

This application also claims benefit of U.S. application Ser. No. 18/046,862 filed Oct. 14, 2022, U.S. application Ser. No. 18/046,875 filed Oct. 14, 2022, and U.S. application Ser. No. 18/046,462 filed Oct. 13, 2022, the specifications of which are incorporated herein in their entirety by reference.

### STATEMENT REGARDING FEDERALLY SPONSORED RESEARCH OR DEVELOPMENT

This invention was made with government support under Grant No. AI158060 awarded by National Institute of Health. The government has certain rights in the invention.

### REFERENCE TO A SEQUENCE LISTING

The contents of the electronic sequence listing (name of the file UCI 22_10 PCT.xml; Size: 592,890,311 bytes; and Date of Creation: Jun. 7, 2023) is herein incorporated by reference in its entirety.

### FIELD OF THE INVENTION

The present invention relates to vaccines, for example, viral vaccines, such as those directed to coronaviruses, e.g., pan-coronavirus vaccines.

### BACKGROUND OF THE INVENTION

While the Wuhan Hu1 variant of SARS-CoV-2 is the ancestral reference virus, Alpha (B.1.1.7), Beta (B.1.351), Gamma or P1 (B.1.1.28.1), and Delta (lineage B.1.617.2) variants of concern (VOCs) subsequently emerged in Brazil, India, and South Africa vaccines from 2020 to 2022. The most recent SARS COV-2 variants, including multiple heavily mutated Omicron (B.1.1.529) sub-variants, have prolonged the COVID-19 pandemic. These new variants emerged since December 2020 at a much higher rate, consistent with the accumulation of two mutations per month, and strong selective pressure on the immunologically important SARS-CoV-2 genes. The Alpha, Beta, Gamma, Delta, and Omicron Variants are defined as Variants of Concern (VOC) based on their high transmissibility associated with increased hospitalizations and deaths. This is a result of reduced neutralization by antibodies generated by previous variants and/or by the first-generation COVID-19 vaccines, together with failures of treatments and diagnostics. Dr. Peter Marks, Director/CBER (Center for Biologics Evaluation and Research) for the FDA recently outlined the need for a next-generation vaccine that will protect from multiple SARS-CoV-2 VOCs.

Besides SARS COV-2 variants, two additional Coronaviruses from the severe acute respiratory syndrome (SARS) like betacoronavirus (sarbecovirus) lineage, SARS coronavirus (SARS-CoV) and MERS-CoV, have caused epidemics and pandemics in humans over the past 20 years. In addition, the discovery of diverse Sarbecoviruses in bats together with the constant “jumping” of these zoonotic viruses from bats to intermediate animals raises the possibility of another COVID pandemic in the future. Hence, there is an urgent need to develop a pre-emptive universal pan-Coronavirus vaccine to protect against all SARS-CoV-2 variants, SARS-CoV, MERS-CoV, and other zoonotic Sarbecoviruses with the potential to jump from animals into humans.

The Spike protein is a surface predominant antigen of SARS-CoV-2 that is involved in the docking and penetration of the virus into the target host cells. As such, the Spike protein is the main target of the first-generation COVID-19 subunit vaccines aiming mainly at inducing neutralizing antibodies. Nearly 56% of the 10 billion doses of first-generation COVID-19 vaccines are based on the Spike antigen alone, while the remaining 44% of the COVID-19 vaccines were based on whole virion inactivated (WVI) vaccines. Both the Spike-based COVID-19 sub-unit vaccines and the whole virion-inactivated vaccines were successful. However, because the Spike protein is the most mutated SARS-CoV-2 antigen, these first-generation vaccines lead to immune evasion by many new variants and subvariants, such as the Omicron XBB1.5 sub-variant. Therefore, the second-generation COVID-19 vaccines should be focused not only on the highly variable Spike protein but also on other highly conserved structural and non-structural SARS-CoV-2 antigens capable of inducing protection mediated by not only neutralizing antibodies but also by cross-reactive CD4⁺ and CD8⁺ T cells.

### BRIEF SUMMARY OF THE INVENTION

It is an objective of the present invention to provide compositions and methods featuring a universal pre-emptive coronavirus vaccine as specified in the independent claims. Embodiments of the invention are given in the dependent claims. Embodiments of the present invention can be freely combined with each other if they are not mutually exclusive.

For example, the present invention features a universal pre-emptive pan-Coronavirus vaccine composition, wherein the composition comprising at least two of: (i) one or more conserved coronavirus B-cell target epitopes selected from SEQ ID NO: 23-31, or a combination thereof; (ii) one or more conserved coronavirus CD4+ T cell target epitopes selected from SEQ ID NO: 17-22, or a combination thereof; and (iii) one or more conserved coronavirus CD8+ T cell target epitopes selected from SEQ ID NO: 1-16, or a combination thereof. An at least one epitope of the composition is derived from a non-spike protein.

In some embodiments, the target epitopes are derived from a SARS-CoV-2 protein selected from a group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein and ORF10 protein.

In some embodiments, one or more of the one or more epitopes may be part of one or more large sequences. The one or more large sequences are highly conserved among human and animal Coronaviruses. In some embodiments, at least one large sequence is a whole protein sequence expressed by SARS-CoV-2, a partial protein sequence expressed by SARS-CoV-2, or a combination thereof.

In some embodiments, the large sequences are selected from Variants of Concern or Variants of Interest. In some embodiments, the one or more large sequences are derived from a whole protein sequence expressed by SARS-CoV-2. In some embodiments, the one or more large sequences are derived from a partial protein sequence expressed by SARS-CoV-2. In some embodiments, the one or more large sequences is derived from a full-length spike glycoprotein. In some embodiments, the one or more large sequences is derived from a partial spike glycoprotein. In some embodiments, the one or more large sequences comprises Spike glycoprotein(S) or a portion thereof, Nucleoprotein or a portion thereof, and protein encoded by ORF1a/b or a portion thereof.

Referring to the embodiments herein, the one or more conserved epitopes may be derived from one or more of: one or more SARS-CoV-2 human strains or variants in current circulation; one or more SARS-CoV-2 variants identified in the future; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. past, current, and future coronavirus outbreaks.

Referring to the embodiments herein, the one or more large sequences may be derived from one or more of: one or more of: one or more SARS-CoV-2 human strains or variants in current circulation; one or more SARS-CoV-2 variants identified in the future; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. past, current, and future coronavirus outbreaks.

In some embodiments, the one or more SARS-CoV-2 human strains or variants in current circulation are selected from: strain B.1.177; strain B.1.160, strain B.1.1.7; strain B.1.351; strain P.1; strain B.1.427/B.1.429; strain B.1.258; strain B.1.221; strain B.1.367; strain B.1.1.277; strain B.1.1.302; strain B.1.525; strain B.1.526, strain S: 677H; strain S: 677P; strain B.1.1.529-Omicron (BA.1); strain B.1.1.529-Omicron (BA.2); and strain B.1.617.2-Delta.

In some embodiments, the one or more coronaviruses that cause the common cold are selected from: 229E alpha coronavirus, NL63 alpha coronavirus, OC43 beta coronavirus, and HKU1 beta coronavirus.

In some embodiments, the vaccine composition protects against disease caused by one or more coronavirus variants or coronavirus subvariants. In some embodiments, the coronavirus variants or coronavirus subvariants comprise past or currently circulating coronavirus variants or coronavirus subvariants wherein the coronavirus variants comprise alpha, beta, gamma, delta, and omicron. In some embodiments, the coronavirus variants or coronavirus subvariants comprise future variants or future subvariants of human and animal coronavirus. In some embodiments, the vaccine composition protects against infection and reinfection of coronavirus variants or coronavirus subvariants. In some embodiments, the coronavirus variants or coronavirus subvariants comprise past or currently circulating coronavirus variants or coronavirus subvariants, wherein the coronavirus variants comprise alpha, beta, gamma, delta, and omicron. In some embodiments, the coronavirus variants or coronavirus subvariants comprise future variants or future subvariants of human and animal coronavirus. In some embodiments, the vaccine composition protects against infection or reinfection of one or more coronavirus variants or coronavirus subvariant. In some embodiments, the vaccine composition protects against infection or reinfection of multiple coronavirus variants or coronavirus subvariants. In some embodiments, the vaccine composition protects against infection or re-infection caused by one coronavirus variants or coronavirus subvariants. In some embodiments, the vaccine composition induces strong and long-lasting protection mediated by antibodies (Abs), CD4+ T helper (Th1) cells, and/or CD8+ cytotoxic T-cells (CTL). In some embodiments, the composition protects against Sarbecoviruses, wherein sarbecoviruses comprise SARS-CoV1 or SARS-CoV2.

Referring to the embodiments herein, the composition may further comprise a T cell attracting chemokine, wherein the T cell attracting chemokine is CCL5, CXCL9, CXCL10, CXCL11, or a combination thereof.

Referring to the embodiments herein, the composition may further comprise a composition that promotes T cell proliferation, wherein the composition that promotes T cell proliferation is IL-7 or IL-15.

One of the unique and inventive technical features of the present invention is the use of highly conserved epitopes. Without wishing to limit the invention to any theory or mechanism, it is believed that the technical feature of the present invention advantageously provides for a universal vaccine composition that will protect from future human outbreaks and deter future zoonosis. None of the presently known prior references or work has the unique inventive technical feature of the present invention.

The present invention also includes a pre-emptive pan-coronavirus vaccine composition comprising: at least two conserved coronavirus antigens selected from: (i) a conserved coronavirus Spike protein; (ii) a conserved coronavirus NSP2 protein; (iii) a conserved coronavirus NSP3 protein; (iv) a conserved coronavirus NSP14 protein; and (v) a conserved coronavirus Nucleoprotein. The aforementioned proteins or antigens may refer to portions of a particular entire protein. Thus, the present invention also includes a pre-emptive pan-coronavirus vaccine composition comprising: at least two conserved coronavirus antigens selected from: (i) a conserved coronavirus Spike protein or a portion thereof; (ii) a conserved coronavirus NSP2 protein or a portion thereof; (iii) a conserved coronavirus NSP3 protein or a portion thereof; (iv) a conserved coronavirus NSP14 protein or a portion thereof; and (v) a conserved coronavirus Nucleoprotein or a portion thereof.

In some embodiments, the composition comprises a conserved coronavirus Spike protein (or a portion thereof); and a conserved coronavirus NSP2 protein (or a portion thereof). In some embodiments, the composition comprises a conserved coronavirus Spike protein (or a portion thereof); and a conserved coronavirus NSP3 protein (or a portion thereof). In some embodiments, the composition comprises a conserved coronavirus Spike protein (or a portion thereof); and a conserved coronavirus NSP14 protein (or a portion thereof). In some embodiments, the composition comprises a conserved coronavirus Spike protein (or a portion thereof); and a conserved coronavirus nucleoprotein (nucleocapsid) (or a portion thereof). In some embodiments, the composition comprises a conserved coronavirus Spike protein (or a portion thereof); a conserved coronavirus NSP2 protein (or a portion thereof); and a conserved coronavirus NSP3 protein (or a portion thereof). In some embodiments, the composition comprises a conserved coronavirus Spike protein (or a portion thereof); a conserved coronavirus NSP2 protein (or a portion thereof); and a conserved coronavirus NSP14 protein (or a portion thereof). In some embodiments, the composition comprises a conserved coronavirus Spike protein (or a portion thereof); a conserved coronavirus NSP2 protein (or a portion thereof); and a conserved coronavirus nucleoprotein (nucleocapsid) (or a portion thereof). In some embodiments, the composition comprises a conserved coronavirus Spike protein (or a portion thereof): a conserved coronavirus NSP3 protein (or a portion thereof); and a conserved coronavirus NSP14 protein (or a portion thereof). In some embodiments, the composition comprises a conserved coronavirus Spike protein (or a portion thereof); a conserved coronavirus NSP3 protein (or a portion thereof); and a conserved coronavirus nucleoprotein (nucleocapsid) (or a portion thereof). In some embodiments, the composition comprises a conserved coronavirus Spike protein (or a portion thereof); a conserved coronavirus NSP14 protein (or a portion thereof); and a conserved coronavirus nucleoprotein (nucleocapsid) (or a portion thereof). In some embodiments, the Spike protein (or a portion thereof) comprises one or more proline substitutions. In some embodiments, the composition comprises a conserved coronavirus NSP2 protein (or a portion thereof); and a conserved coronavirus NSP3 protein (or a portion thereof). In some embodiments, the composition comprises a conserved coronavirus NSP2 protein (or a portion thereof); and a conserved coronavirus NSP14 protein (or a portion thereof). In some embodiments, the composition comprises a conserved coronavirus NSP2 protein (or a portion thereof); and a conserved coronavirus nucleoprotein (nucleocapsid) (or a portion thereof). In some embodiments, the composition comprises a conserved coronavirus NSP3 protein (or a portion thereof); and a conserved coronavirus NSP14 protein (or a portion thereof). In some embodiments, the composition comprises a conserved coronavirus NSP3 protein (or a portion thereof); and a conserved coronavirus nucleoprotein (nucleocapsid) (or a portion thereof). In some embodiments, the composition comprises a conserved coronavirus NSP14 protein (or a portion thereof); and a conserved coronavirus nucleoprotein (nucleocapsid) (or a portion thereof). In some embodiments, the composition comprises a conserved coronavirus NSP2 protein (or a portion thereof); a conserved coronavirus NSP3 protein (or a portion thereof); and a conserved coronavirus NSP14 protein (or a portion thereof). In some embodiments, the composition comprises a conserved coronavirus NSP2 protein (or a portion thereof); a conserved coronavirus NSP3 protein (or a portion thereof); and a conserved coronavirus nucleoprotein (nucleocapsid) (or a portion thereof). In some embodiments, the composition comprises a conserved coronavirus NSP2 protein (or a portion thereof); a conserved coronavirus NSP14 protein (or a portion thereof); and a conserved coronavirus nucleoprotein (nucleocapsid) (or a portion thereof). In some embodiments, the composition comprises a conserved coronavirus NSP3 protein (or a portion thereof); a conserved coronavirus NSP14 protein (or a portion thereof); and a conserved coronavirus nucleoprotein (nucleocapsid) (or a portion thereof). In some embodiments, the composition comprises a conserved coronavirus Spike protein (or a portion thereof); a conserved coronavirus NSP2 protein (or a portion thereof): a conserved coronavirus NSP3 protein (or a portion thereof); and a conserved coronavirus NSP14 protein (or a portion thereof). In some embodiments, the composition comprises a conserved coronavirus Spike protein (or a portion thereof); a conserved coronavirus NSP2 protein (or a portion thereof): a conserved coronavirus NSP3 protein (or a portion thereof); and a conserved coronavirus nucleoprotein (nucleocapsid) (or a portion thereof). In some embodiments, the composition comprises a conserved coronavirus Spike protein (or a portion thereof); a conserved coronavirus NSP2 protein (or a portion thereof); a conserved coronavirus NSP3 protein (or a portion thereof); and a conserved coronavirus NSP14 protein (or a portion thereof): a conserved coronavirus nucleoprotein (nucleocapsid) (or a portion thereof). In some embodiments, the Spike protein (or a portion thereof) comprises one or more proline substitutions. In some embodiments, the composition comprises a conserved coronavirus NSP2 protein (or a portion thereof); a conserved coronavirus NSP3 protein (or a portion thereof); and a conserved coronavirus NSP14 protein (or a portion thereof); a conserved coronavirus nucleoprotein (nucleocapsid) (or a portion thereof).

In some embodiments, the composition further comprises a T cell attracting chemokine, wherein the T cell attracting chemokine is CCL5, CXCL9, CXCL10, CXCL11, or a combination thereof. In some embodiments, the composition further comprises a composition that promotes T cell proliferation and T-cell memory, wherein the composition that promotes T cell proliferation and memory is IL-7, IL-2, or IL-15. In some embodiments, the conserved protein or antigen is conserved among human and animal coronaviruses. In some embodiments, the portion of the coronavirus spike(S) protein is derived from a full-length spike glycoprotein. In some embodiments, the portion of the coronavirus spike(S) protein is derived from a partial spike glycoprotein. In some embodiments, the portion of the coronavirus spike(S) protein is receptor-binding domain (RBD). In some embodiments, the RBD comprises a trimerized SARS-CoV-2 receptor-binding domain (RBD).

The present invention also includes a pre-emptive pan-coronavirus vaccine composition, the composition comprising one or more large sequence coronavirus proteins, wherein the one or more large sequence coronavirus proteins comprise one or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus nucleoprotein (nucleocapsid) (or a portion thereof).

In some embodiments, the one or more large sequence coronavirus proteins comprises two or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus nucleoprotein (nucleocapsid) (or a portion thereof). In some embodiments, the one or more large sequence coronavirus proteins comprises three or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus nucleoprotein (nucleocapsid) (or a portion thereof). In some embodiments, the one or more large sequence coronavirus proteins comprises four or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus nucleoprotein (nucleocapsid) (or a portion thereof). In some embodiments, the one or more large sequence coronavirus proteins comprises: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); and a conserved coronavirus nucleoprotein (nucleocapsid) (or a portion thereof). In some embodiments, the large coronavirus sequences are highly conserved among human and animal coronaviruses. In some embodiments, the Spike(S) protein further comprises at least one proline substitution. In some embodiments, the Spike(S) protein comprises a receptor-binding domain (RBD). In some embodiments, the RBD comprises a trimerized SARS-CoV-2 receptor-binding domain (RBD). In some embodiments, the composition further comprises a T cell attracting chemokine, wherein the T cell attracting chemokine is CCL5, CXCL9, CXCL10, CXCL11, or a combination thereof. In some embodiments, the composition further comprises a composition that promotes T cell proliferation and T-cell memory, wherein the composition that promotes T cell proliferation and memory is IL-7, IL-2, or IL-15.

The present invention also features a pre-emptive pan-coronavirus vaccine composition, the composition comprising, or comprising a sequence encoding one or more large sequence coronavirus proteins, wherein the one or more large sequence coronavirus proteins comprise: a conserved coronavirus Spike protein; a conserved coronavirus NSP2 protein; a conserved coronavirus NSP14 protein; and a conserved coronavirus Nucleoprotein.

The present invention also features a method of preventing infection or reinfection by one or more coronavirus variants or subvariants in a subject, said method comprising administering a therapeutically effective amount of a composition according to the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B shows the evolution of emerging SARS-CoV-2 Variants of Concerns and their genetic similarity with other human and animal coronaviruses: FIG. 1A shows a dendrogram showing the evolution of SARS-CoV-2 lineages based on PANGO and Nextstrain nomenclature (top panel). Phylogenetic analysis performed between 712,000 human SARS-CoV-2 genome sequences to show degree of genetic similarity among different SARS-CoV-2 lineages evolved between December 2019 and April 2021 (bottom panel). FIG. 1B shows phylogenetic analysis performed among VOCs of SARS-CoV-2, CoVs strains from human and other species showed minimum genetic distance between the first SARS-CoV-2 isolate Wuhan-Hu-1 reported from the Wuhan Seafood market with different VOCs, followed by bat strains hCoV-19-bat-Yunnan-RmYN02, hCoV-19-bat-Yunnan-RaTG13, and bat-CoV-19-ZXC21 (top panel). Genetic distances based on Maximum Composite Likelihood model among the human, bat, pangolin, civet cat and camel genome sequences. Results indicate least genetic distance among SARS-CoV-2 isolated Wuhan-Hu-1 with VOCs EU2, CAL20C, 20B, B.1.17, P.1, and 20C B.1.367 (bottom panel).

FIGS. 2A and 2B shows identification of SARS-CoV-2 CD8+ T cell epitopes specific to spike glycoprotein possessing non-synonymous mutations from VOCs. FIG. 2A shows twenty-six, spike glycoprotein specific epitopes were predicted. The spike glycoprotein is substituted with all the known non-synonymous mutations reported in context to the 17 SARS-CoV-2 specific variants of concern. FIGS. 2B and 2C show the SARS-CoV-2-derived CD8+ T cell epitopes are screened based on their presence among most frequently observed HLA-A alleles in global population (HLA-A*01:01, HLA-A*02:01, HLA-A*03:01, HLA-A*11:01, HLA-A*23:01). MHC-I binding affinity and high degree of immunogenicity showed high PPC value of 91.48%, average number of epitope hits/HLA combinations recognized by the population is 21.3, and minimum number of epitope hits/HLA combinations recognized by 90% of the population (pc90) is 5.06.

FIGS. 3A, 3B, and 3C shows the identification of SARS-CoV-2 CD4+ T cell epitopes specific to spike glycoprotein possessing non-synonymous mutations from VOCs. FIG. 3A shows Nineteen spike glycoprotein specific CD4+ T cell epitopes were predicted. The spike glycoprotein is substituted with all the known non-synonymous mutations reported in context to the 17 SARS-CoV-2 specific variants of concern. FIGS. 3B and 3C shows the SARS-CoV-2-derived CD4+ T cell epitopes are screened based on their presence among most frequently observed HLA-DR alleles in global population (HLA-DRB1*01:01, HLA-DRB1*11:01, HLA-DRB1*15:01, HLA-DRB1*03:01, HLADRB1*04:01). MHC-I binding affinity and high degree of immunogenicity showed high PPC value of 86.39%, average number of epitope hits/HLA combinations recognized by the population is 13.69, and minimum number of epitope hits/HLA combinations recognized by 90% of the population (pc90) is 2.87.

FIGS. 4A and 4B shows the docking of SARS-CoV-2 Spike glycoprotein-derived B cell epitopes to human ACE2 receptors. FIG. 4A shows eight spike glycoprotein specific B cell epitopes were predicted. The spike glycoprotein is substituted with all the known non-synonymous mutations reported in context to the 17 SARS-CoV-2 specific variants of concern. FIG. 4B shows molecular docking of 8 B-cell epitopes, identified from the SARS-CoV-2 Spike glycoprotein, with ACE2 receptors. B cell epitope peptides are shown in ball and stick structures whereas the ACE2 receptor protein is shown as a template.

FIGS. 5A, 5B, and 5C shows experimental evidence showing immunization with pool of CD8+ T cell, CD4+ T cell, and B cell peptides to provide protection against all the current SARS-CoV-2 variants of concerns in triple transgenic h-ACE2-HLA-A2/DR mice. Viral titration (FIG. 5A), weight loss (FIG. 5B), and survival (FIG. 5C) data have been shown.

FIG. 6 shows non-limiting configurations of vaccine compositions described herein.

FIGS. 7A and 7B shows non-limiting examples of hybrid vaccine compositions described herein. The proteins may be covalently or non-covalently linked together for administration of the vaccine composition. Note: “Nsp” may refer to Nsp1, Nsp2, Nsp3, Nsp5, Nsp6, Nsp7, Nsp8, Nsp9, Nsp10, Nsp12, Nsp13, Nsp14, Nsp15, Nsp16, or a combination thereof, “Spike protein” may refer to a portion of the spike protein, or a spike protein with one or more mutations (e.g., a spike protein with two or six proline substitutions).

FIG. 8 shows sequence-based variant effect predictor analysis to access the pathogenicity of mutations in VOCs.

FIGS. 9A, 9B, and 9C show screening of COVID-19 patients based on SARS-CoV-2 variants and subsequent evaluation of IFN-γ CD8⁺ and CD4⁺ T cell responses for conserved CD8⁺, and CD4⁺ T cell “asymptomatic” epitopes: FIG. 9A shows an experimental plan of the screening process of COVID-19 patients (n=210) into Asymptomatic and Symptomatic categories based on clinical parameters. Blood and nasopharyngeal swabs were collected from all the subjects and a qRT-PCR assay was performed. Six novel nonsynonymous mutations (Δ69-70, Δ242-244, N501Y, E484K, L452R, and T478K) were used to identify the haplotypes unique to different SARS-CoV-2 variants of concern (Omicron (B.1.1.529 (BA.1)), Omicron (B.1.1.529 (BA.2)), Alpha (B.1.1.7), Beta (B.1.351), Gamma (P.1), Delta (B.1.617.2), and Epsilon (B.1.427/B.1.429)) and variants of interest (Eta (B.1.525), R.1, Zeta (P.2), lota (B.1.526) and B.1.2/501Y or B.1.1.165). FIG. 9B shows ELISpot images and bar diagrams showing average frequencies of IFN-γ producing cell spots from immune cells from PBMCs (1×10⁶ cells per well) of COVID-19 infected with highly pathogenic SARS-CoV-2 variants of concern Beta (B.1.351) (left panel) and Omicron (B.1.1.529) (right panel). Cells were stimulated for 48 hours with 10 mM of 16 immunodominant CD8+ T cell peptides derived from SARS-CoV-2 structural (Spike, Envelope, Membrane) and nonstructural (orf1ab, ORF6, ORF7b, ORF8a, ORF10) proteins. FIG. 9C shows ELISpot images and bar diagrams showing average frequencies of IFN-γ producing cell spots from immune cells from PBMCs (1×10⁶ cells per well) of COVID-19 infected with SARS-CoV-2 variants of concern Alpha (B.1.1.7) (left panel) and Omicron (B.1.1.529) (right panel). Cells were stimulated for 48 hours with 10 mM of 6 immunodominant CD4⁺ T cell peptides derived from SARS-CoV-2 structural (Spike, Membrane, Nucleocapsid) and nonstructural (ORF1a, ORF6, ORF8a) proteins. The bar diagrams show the average/mean numbers (±SD) of IFN-γ-spot forming cells (SFCs) after CD8⁺ T cell peptide-stimulation PBMCs of Asymptomatic and Symptomatic COVID-19 patients. Dotted lines represent an arbitrary threshold set to evaluate the relative magnitude of the response. A strong response is defined for mean SFCs>25 per 1×10⁶ stimulated PBMCs. Results were considered statistically significant at P<0.05.

FIGS. 10A, 10B, 10C, and 10D shows protection induced against six SARS-CoV-2 variants of concern in triple transgenic HLA-A*02:01/HLA-DRB1*01:01-hACE-2 mice following immunization with a pan-Coronavirus vaccine incorporating conserved human B, CD4+, and CD8+ T cell “asymptomatic” epitopes: FIG. 10A shows an experimental scheme of vaccination and challenge triple transgenic HLA-A*02:01/HLA-DRB1*01:01-hACE-2 mice. Triple transgenic HLA-A*02:01/HLA-DRB1*01:01-hACE-2 mice (7-8-week-old, n=60) were immunized subcutaneously on Days 0 and 14 with a multi-epitope pan-Coronavirus vaccine consisting of a pool of conserved B, CD4⁺ T cell and CD8⁺ T cell human epitope peptides. The pool of peptides comprised 25 μg of each of the 16 CD8⁺ T cell peptides, 6 CD4⁺ T cell peptides, and 7 B-cell peptides. The final composition of peptides was mixed with 25 μg of CpG and 25 μg of Alum. Mock-vaccinated mice were used as controls (Mock). Fourteen days following the second immunization, mice were intranasally challenged with each of the six different SARS-CoV-2 variants of concern (WA/USA2020, Alpha (B.1.1.7), Beta (B.1.351), Gamma (P.1), Delta (B.1.617.2), and Omicron (B.1.1.529)). Vaccinated and mock-vaccinated mice were followed 14 days post-challenge for COVID-like symptoms, weight loss, survival, and virus replication. FIG. 10B shows the percent weight change recorded daily for 14 days p.i. in vaccinated and mock-vaccinated mice following the challenge with each of the six different SARS-CoV-2 variants. FIG. 10C shows kaplan-Meir survival plots for vaccinated and mock-vaccinated mice following the challenge with each of the six different SARS-CoV-2 variants. FIG. 10D shows virus replication in vaccinated and mock-vaccinated mice following the challenge with each of the six different SARS-CoV-2 variants detected in throat swabs on Days 2, 4, 6, 8, 10, and 14, The indicated P values are calculated using the unpaired t-test, comparing results obtained in vaccinated VERSUS mock-vaccinated mice.

FIGS. 11A, 11B, 11C, and 11D shows histopathology and immunohistochemistry of the lungs from in triple transgenic HLA-A*02:01/HLA-DRB1*01:01-hACE-2 mice vaccinated and mock-vaccinated mice. FIG. 11A shows representative images of hematoxylin and Eosin (H & E) staining of the lungs harvested on day 14 p.i. from vaccinated (left panels) and mock-vaccinated (right panels) mice. FIG. 11B shows a representative immunohistochemistry (IHC) sections of the lungs were harvested on Day 14 p.i. from vaccinated (left panels) and mock-vaccinated (right panels) mice and stained with SARS-CoV-2 Nucleocapsid antibody. Black arrows point to the antibody staining. Fluorescence microscopy images showing infiltration of CD8⁺ T cells (FIG. 11C) and of CD4⁺ T cells (FIG. 11D) in the lungs from vaccinated (left panels) and mock-vaccinated (right panels) mice. Lung sections were co-stained using DAPI (blue) and mAb specific to CD8⁺ T cells (Pink) (magnification, 20×). The white arrows point to CD8⁺ and CD4⁺ T cells infiltrating the infected lungs.

FIGS. 12A, 12B, 12C, and 12D shows the effect of Pan-Coronavirus immunization on CD8⁺ and CD4⁺ T cell function and memory response: FACS plots and bar graphs showing the (FIG. 12A) expression of CD8⁺ T cell function markers, (FIG. 12B) CD4⁺ T cell function associated markers, (FIG. 12C) CD8⁺ T effector memory response (CD44⁺CD62L⁻), and CD8″ T resident memory (CD103⁺CD69⁺) response, and (FIG. 12D) CD4⁺ T effector memory response (CD44⁺CD62L⁻), and CD4⁺ resident memory (CD103⁺CD69⁺) response in the lung of vaccinated and mock-vaccinated groups of mice infected with multiple SARS-CoV-2 variants. Bars represent means±SEM. Data were analyzed by student's t-test. Results were considered statistically significant at P<0.05.

FIGS. 13A, 13B, 13C, 13D, 13E, and 13F shows Immunogenicity of conserved SARS-CoV-2 CD8⁺ T cell epitopes in triple transgenic HLA-A*02:01/HLA-DRB1*01:01-hACE-2 mice: ELISpot images and bar diagrams showing average frequencies of IFN-producing cell spots from mononuclear cells from lung tissue (1×10⁶ cells per well) of vaccinated and mock-vaccinated mice challenged with (FIG. 13A) WA/USA2020, (FIG. 13B) Alpha (B.1.1.7), (FIG. 13C) Beta (B.1.351), (FIG. 13D) Gamma (P.1), (FIG. 13E) Delta (B.1.617.2), and (FIG. 13F) Omicron (B.1.1.529). The cells were stimulated for 48 hours with 10 mM of 16 immunodominant CD8+ T cell peptides. The bar diagrams show the average/mean numbers (+SD) of IFN-γ-spot forming cells (SFCs) after CD8⁺ T cell peptide stimulation in lung tissues of vaccinated and mock-vaccinated mice. Dotted lines represent an arbitrary threshold set to evaluate the relative magnitude of the response. A strong response is defined for mean SFCs>25 per 1×10⁶ stimulated PBMCs. Results were considered statistically significant at P<0.05.

FIGS. 14A, 14B, 14C, 14D, 14E, and 14F show The magnitude of the IFN-CD4⁺ T cell responses for 6 conserved SARS-CoV-2 CD4+ T cell epitopes in triple transgenic HLA-A*02:01/HLA-DRB1*01:01-hACE-2 mice: ELISpot images and bar diagrams showing average frequencies of IFN-producing cell spots from mononuclear cells from lung tissue (1×10⁶ cells per well) of vaccinated and mock-vaccinated mice challenged with (FIG. 14A) WA/USA2020, (FIG. 14B) Alpha (B.1.1.7), (FIG. 14C) Beta (B.1.351), (FIG. 14D) Gamma (P.1), (FIG. 14E) Delta (B.1.617.2), and (FIG. 14F) Omicron (B.1.1.529). Cells were stimulated for 48 hours with 10 mM of 6 immunodominant CD4+ T cell peptides derived from SARS-CoV-2 structural (Spike, Envelope, Membrane) and nonstructural (orf1ab, ORF6, ORF7b, ORF8a, ORF10) proteins. The bar diagrams show the average/mean numbers (+SD) of IFN-γ-spot forming cells (SFCs) after CD8+ T cell peptide stimulation in lung tissues of vaccinated and mock-vaccinated mice. The dotted lines represent an arbitrary threshold set to evaluate the relative magnitude of the response. A strong response is defined for mean SFCs>25 per 1×10⁶ stimulated PBMCs. Results were considered statistically significant at P≤0.05.

FIGS. 15A, 15B, 15C, and 15D show the effect of treatment with CXCL-9, CXCL-10, and CXCL-11 chemokines on COVID-19-like symptoms detected from K18-hACE2 single transgenic mice infected with SARS-CoV-2. (FIG. 15A) Experimental plan to study the effect of treatment with CXCL-9, CXCL-10, and CXCL-11 chemokines on COVID-19-like symptoms detected from K18-hACE2 single transgenic mice following infection with SARS-CoV-2 (Washington USA-WA1/2020 variant). 8-9 week-old male and female K18-hACE2 mice (n=20) were infected intranasally with 1×10⁴ pfu of the SARS-CoV-2-USA-WA1/2020 variant. Mice were subsequently treated intranasally with CXCL-9 (n=4), CXCL-10 (n=4), and CXCL-11 (n=4) on days 3, 5, 7, and 9 post-infection (p.i). As a control mice (n=4) were left untreated. 15D shows the average body weight change p.i. normalized to the body weight on the day of infection. FIG. 15B shows the maximum percent body weight change on day 7 p.i. (FIG. 15C) Shows the percentage survival detected in CXCL-9, CXCL-10, and CXCL-11 treated vs. untreated mice up to day 13 p.i.

FIGS. 16A and 16B shows the effect of treatment with CXCL-9, CXCL-10, and CXCL-11chemokines on disease outcome detected from HLA-DR*0101/HLA-A*0201/hACE2 triple transgenic mice immunized with SARS-CoV-2 vaccine. (FIG. 16A) FIG. 16B shows a prototype of the multi-epitope Coronavirus vaccine consisting of highly conserved and immunogenic 16 CD8+ T cell epitopes, 6 CD4+ T cell epitopes, and 8 B cell epitopes (SEQ ID NO: 36). Experimental plan to study the effect of treatment with CXCL-9, CXCL-10, and CXCL-11 chemokines on COVID-19-like symptoms detected from HLA-DR*0101/HLA-A*0201/hACE2 triple transgenic mice immunized with multi-epitope Coronavirus vaccine (CoV-Vacc). 8-9 week-old male and female HLA-DR*0101/HLA-A*0201/hACE2 triple transgenic mice were intranasally immunized with CoV-Vacc; 2×10¹⁰ VP per mice on day 0 (n=28). As a control mice (n=7) were left unimmunized. The immunized mice were subsequently treated intranasally with 2.4 μg of; CXCL-9 (n=7), CXCL-10 (n=7), and CXCL-11 (n=7) on days 10,12,14,22,24,26 post-immunization. On day 27 two mice per group were euthanized, and lung tissues were collected. The immune cell response was evaluated by flow cytometry. The remaining mice (n=25) were intranasally infected with 1×10⁴ pfu of SARS-CoV-2 (USA-WA1/2020) on day 28 post-immunization. Three mice groups were subsequently treated intranasally with CXCL-9 (n=5), CXCL-10 (n=5), and CXCL-11 (n=5) on days 30, 32, and 34 post-immunization. disease monitoring, weighing, and survival were monitored in the mice up to day 14 p.i.

FIGS. 17A, 17B, 17C, and 17D shows the effect of treatment with CXCL-9, CXCL-10, and CXCL-11 chemokines on COVID-19-like symptoms detected from HLA-DR*0101/HLA-A*0201/hACE2 triple transgenic mice immunized with multi-epitope Coronavirus and challenged with SARS-CoV-2-USA-WA1/2020 variant. (FIG. 17A) Data showing average percent weight change each day p.i. normalized to the body weight on the day of infection is shown in the right panel. The bar graph (left panel) shows the percent weight change at day 7 p.i. Bars represent means±SEM. (FIG. 17B) Shows the percentage survival detected in mice groups of, CoV-Vacc, CoV-Vacc+CXCL-9, CoV-Vacc+CXCL-10, CoV-Vacc+CXCL-11, and mock vaccinated group up to 14 days p.i. (FIG. 17C) Viral titration data showing viral RNA copy number in the lungs for each group at days 4 and 8 p.i. (FIG. 17D) Representative H & E staining images of the lungs at day 14 p.i. of SARS-CoV-2 infected mice treated with different chemokines at 4×, 10×, and 40× magnifications.

FIGS. 18A and 18B shows the effect of treatment with CXCL-9, CXCL-10, and CXCL-11chemokines on CD4⁺ T cells in the lung and spleen of immunized HLA-DR*0101/HLA-A*0201/hACE2 triple transgenic mice. (FIG. 18A) The left panel shows FACS plots for CD4⁺ T cells in the lungs of mice immunized with the multi-epitope Coronavirus vaccine (n=7) and treated with CXCL-9, CXCL-10, and CXCL-11. Graphs depict the differences in response to various treatments on the percentage of CD4⁺ T cells present in the lungs of mice shown in the right panel. Bars represent the means±SEM. Student's t-test was used to analyze the data. (FIG. 18B) The left panel represents FACS plots for CD4⁺ T cells in the spleen of mice immunized with the multi-epitope Coronavirus vaccine (n=7) and treated with CXCL-9, CXCL-10, and CXCL-11. Graphs on the right panel show the difference in response to different chemokine treatments on the percentage of CD4+ T cells in the spleen of mice. Bars represent means±SEM.

FIGS. 19A and 19B shows the effect of treatment with CXCL-9, CXCL-10, and CXCL-11chemokines on CD8⁺ T cells in the lung and spleen of immunized hACE2-HLA-A2/DR triple transgenic mice. (FIG. 19A) The left panel shows FACS plots for CD8⁺ T cells in the lungs of mice immunized with the multi-epitope Coronavirus vaccine (n=7) and treated with CXCL-9, CXCL-10, and CXCL-11. Graphs depict the differences in response to various treatments on the percentage of CD8⁺ T cells present in the lungs of mice shown in the right panel. Bars represent the means±SEM. Student's t-test was used to analyze the data. (FIG. 19B) The left panel represents FACS plots for CD8+ T cells in the spleen of mice immunized with the multi-epitope Coronavirus vaccine (n=7) and treated with CXCL-9, CXCL-10, and CXCL-11. Graphs on the right panel show the difference in response to different chemokine treatments on the percentage of CD4⁺ T cells in the spleen of mice. Bars represent means±SEM.

FIGS. 20A and 20B shows the effect of treatment with CXCL-9, CXCL-10, and CXCL-11chemokines on CD4⁺ T cells and CD8⁺ T cells in the lung of immunized hACE2-HLA-A2/DR triple transgenic mice. (FIG. 20A) The left panel shows FACS plots for CD4⁺ T cells in the lungs of mice immunized with the multi-epitope Coronavirus vaccine (n=5) and treated with CXCL-9, CXCL-10, and CXCL-11. Graphs depict the differences in response to various treatments on the percentage of CD4⁺ T cells present in the lungs of mice shown in the right panel. Bars represent the means±SEM. Student's t-test was used to analyze the data. (FIG. 20B) The left panel represents FACS plots for CD8⁺ T cells in the lungs of mice immunized with the multi-epitope Coronavirus vaccine (n=5) and treated with CXCL-9, CXCL-10, and CXCL-11. Graphs on the right panel show the difference in response to different chemokine treatments on the percentage of CD8⁺ T cells in the lungs of mice. Bars represent means±SEM.

FIGS. 21A, 21B, and 21C shows an experimental schematic (FIG. 21A) to determine the efficacy of mRNA-LNP vaccine against the SARS-CoV-2 Delta (B.1.617.2) (FIG. 21B) and Omicron (B.1.1.529) (FIG. 21C) variants in golden hamster.

FIG. 22 shows qRT-PCR data showing reduced viral copy number for hamsters immunized with different mRNA-LNP Pan-CoV-Vaccines against the SARS-CoV-2 Delta (B.1.617.2) variant in golden hamsters

FIG. 23 shows qRT-PCR data showing reduced viral copy number for hamsters immunized with different mRNA-LNP Pan-CoV-Vaccines against the SARS-CoV-2 Delta (B.1.617.2) variant in golden hamsters.

FIG. 24 shows RT-PCR data showing reduced viral copy number for hamsters immunized with different mRNA-LNP Pan-CoV-Vaccines against the SARS-CoV-2 Delta (B.1.617.2) variant in golden hamsters.

FIG. 25 shows qRT-PCR data showing reduced viral copy number for hamsters immunized with different mRNA-LNP Pan-CoV-Vaccines against the SARS-CoV-2 Delta (B.1.617.2) variant in golden hamsters.

FIGS. 26A and 26B shows Anti SARS-CoV-2 Spike specific IgG measured in the serum of immunized hamsters with mRNA-LNP 2 proline versus 6 proline vaccine at 1 μg and 10 μg. FIG. 26A show an endpoint titer is statistically significant increase in spike specific IgG antibody in the serum of hamsters immunized with Spike 2 proline (1 μg) compared to Spike 6 proline (1 μg). (blood was collected at day 27 after the first immunization (FIG. 21). FIG. 26B shows endpoint titer has no statistical significant difference in spike specific IgG antibody in the serum of hamsters immunized with Spike 2 proline (10 μg) compared to Spike 6 proline (10 μg) (blood was collected at day 27 after the first immunization) (FIG. 21). Plates were coated with 100 ng of Spike (S1+S2) from Sino biological

FIG. 27 shows qRT-PCR data showing reduced viral copy number for hamsters immunized with different mRNA-LNP Pan-CoV-Vaccines against the SARS-CoV-2 Omicron (B.1.1.529) variant in golden hamsters.

FIG. 28 shows qRT-PCR data showing reduced viral copy number for hamsters immunized with different mRNA-LNP Pan-CoV-Vaccines against the SARS-CoV-2 Omicron (B.1.1.529) variant in golden hamsters.

FIG. 29 shows qRT-PCR data showing reduced viral copy number for hamsters immunized with different mRNA-LNP Pan-CoV-Vaccines against the SARS-CoV-2 Omicron (B.1.1.529) variant in golden hamsters.

FIG. 30 shows qRT-PCR data showing reduced viral copy number for hamsters immunized with different mRNA-LNP Pan-CoV-Vaccines against the SARS-CoV-2 Omicron (B.1.1.529) variant in golden hamsters.

FIGS. 31A, 31B, and 31C shows an experimental schematic to determine the efficacy of mRNA-LNP vaccine against the SARS-CoV-2 variants of concern (Delta (B.1.617.2) FIG. 31A) and Omicron (B.1.1.529) FIG. 31C) in golden hamster

FIG. 32 shows qRT-PCR data showing reduced viral copy number for hamsters immunized with different mRNA-LNP Pan-CoV-Vaccines against the SARS-CoV-2 Delta (B.1.617.2) variant in golden hamsters.

FIG. 33 shows qRT-PCR data showing reduced viral copy number for hamsters immunized with different mRNA-LNP Pan-CoV-Vaccines against the SARS-CoV-2 Delta (B.1.617.2) variant in golden hamsters.

FIG. 34 shows qRT-PCR data showing reduced viral copy number for hamsters immunized with different mRNA-LNP Pan-CoV-Vaccines against the SARS-CoV-2 Delta (B.1.617.2) variant in golden hamsters.

FIG. 35 shows qRT-PCR data showing reduced viral copy number for hamsters immunized with different mRNA-LNP Pan-CoV-Vaccines against the SARS-CoV-2 Delta (B.1.617.2) variant in golden hamsters.

FIG. 36 shows qRT-PCR data showing reduced viral copy number for hamsters immunized with different mRNA-LNP Pan-CoV-Vaccines against the SARS-CoV-2 Delta (B.1.617.2) variant in golden hamsters.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely.” Furthermore, variations of the word “comprising,” such as “comprise” and “comprises,” have correspondingly the same meanings. In one respect, the technology described herein is related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and are not intended to be limiting.

As used herein, the term “immunological response” to a composition or vaccine refers to the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. The host may display either a therapeutic or protective immunological response, so resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the term “variant” refers to a substantially similar sequence. For polynucleotides, a variant comprises a deletion and/or addition and/or change of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or an amino acid sequence, respectively. Variants of a particular polynucleotide of the disclosure (e.g., the reference polynucleotide) can also be evaluated by comparison of the percent sequence identity between the polypeptide encoded by a variant polynucleotide and the polypeptide encoded by the reference polynucleotide. “Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active; that is they have the ability to elicit an immune response.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

As used herein, the term “carrier,” or “pharmaceutically acceptable carrier,” or “pharmaceutically acceptable vehicle” refers to any appropriate or useful carrier or vehicle for introducing a composition to a subject. Pharmaceutically acceptable carriers or vehicles may be conventional but are not limited to conventional vehicles. For example, E. W. Martin, Remington's Pharmaceutical Sciences, Mack Publishing Co., Easton, PA, 15th Edition (1975) and D. B. Troy, ed. Remington: The Science and Practice of Pharmacy, Lippincott Williams & Wilkins, Baltimore MD and Philadelphia, PA, 21^{st} Edition (2006) describe compositions and formulations suitable for pharmaceutical delivery of one or more therapeutic compounds or molecules. Carriers (e.g., pharmaceutical carriers, pharmaceutical vehicles, pharmaceutical compositions, pharmaceutical molecules, etc.) are materials generally known to deliver molecules, proteins, cells and/or drugs and/or other appropriate material into the body. In general, the nature of the carrier will depend on the nature of the composition being delivered as well as the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like. Patents that describe pharmaceutical carriers include, but are not limited to: U.S. Pat. Nos. 6,667,371; 6,613,355; 6,596,296; 6,413,536; 5,968,543; 4,079,038; 4,093,709; 4,131,648; 4,138,344: U.S. Pat. Nos. 4,180,646; 4,304,767; 4,946,931, the disclosures of which are incorporated in their entirety by reference herein. The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise a biological matrix and a portion may comprise synthetic matrix.

As used herein, “coronavirus” may refer to a group of related viruses such as but not limited to severe acute respiratory syndrome (SARS), middle east respiratory syndrome (MERS), and severe acute respiratory syndrome coronavirus 2 (SARS-CoV-2). All the coronaviruses cause respiratory tract infection that range from mild to lethal in mammals. Several non-limiting examples of Coronavirus strains are described herein.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile, or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects

The terms “administering,” and “administration” refer to methods of providing a pharmaceutical preparation to a subject. Such methods are well known to those skilled in the art and include, but are not limited to, administering the compositions orally, parenterally (e.g., intravenously, and subcutaneously), by intramuscular injection, by intraperitoneal injection, intrathecally, transdermally, extracorporeally, topically or the like.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition comprising the vaccine composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the vaccine composition is placed between the gum and the cheek of a patient. As used herein “sublingual delivery” may refer to a method of administration in which the compound is delivered through the mucosal membrane under the tongue. In some embodiments, for a sublingual delivery the vaccine composition is administered under the tongue of a patient.

Parenteral administration of the composition, if used, is generally characterized by injection.

Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. Embodiments of the present invention can be freely combined with each other if they are not mutually exclusive

### Multi-Epitope Vaccines:

The present invention features preemptive multi-epitope pan-Coronavirus vaccines, methods of use, and methods of producing said vaccines, methods of preventing coronavirus infections, etc. The present invention also provides methods of testing said vaccines, e.g., using particular animal models and clinical trials. The vaccine compositions herein can induce efficient and powerful protection against the coronavirus disease or infection, e.g., by inducing the production of antibodies (Abs), CD4+ T helper (Th1) cells, and CD+8 cytotoxic T-cells (CTL).

The vaccine compositions, e.g., the antigens, herein feature multiple epitopes, which helps provide multiple opportunities for the body to develop an immune response for preventing an infection.

In certain embodiments, the epitopes are conserved epitopes, e.g., epitopes that are highly conserved among human coronaviruses and/or animal coronaviruses (e.g., coronaviruses isolated from animals susceptible to coronavirus infections). The vaccines herein may be designed to be effective against past, current, and future coronavirus outbreaks

As used herein, the term “conserved” refers to an epitope that is among the most highly conserved epitopes identified in a sequence alignment and analysis for its particular epitopes type (e.g., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). In some embodiments, the conserved epitopes may be the 50% most highly conserved epitopes identified (for the particular type of epitope). In some embodiments, the conserved epitopes may be the 60% most highly conserved epitopes identified (for the particular type of epitope). In some embodiments, the conserved epitopes may be the 70% most highly conserved epitopes identified (for the particular type of epitope). In some embodiments, the conserved epitopes may be the 80% most highly conserved epitopes identified (for the particular type of epitope). In some embodiments, the conserved epitopes may be the 90% most highly conserved epitopes identified (for the particular type of epitope). In some embodiments, the conserved epitopes may be the 95% most highly conserved epitopes identified (for the particular type of epitope). In some embodiments, the conserved epitopes may be the 99% most highly conserved epitopes identified (for the particular type of epitope). The present invention is not limited to the aforementioned thresholds.

In certain embodiments, one or more of the conserved epitopes are derived from one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. SARS-CoV-2 human strains and variants in current circulation may include the original SARS-CoV-2 strain (SARS-CoV-2 isolate Wuhan-Hu-1), and several variants of SARS-CoV-2 including but not limited to Spain variant B.1.177: Australia variant B.1.160, England variant B.1.1.7; South Africa variant B.1.351; Brazil variant P.1; California variant B.1.427/B.1.429; Scotland variant B.1.258; Belgium/Netherlands variant B.1.221; Norway/France variant B.1.367; Norway/Denmark.UK variant B.1.1.277; Sweden variant B.1.1.302; North America, Europe, Asia, Africa, and Australia variant B.1.525; a New York variant B.1.526; variant B.1.525; variant B.1.526, variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). The present invention is not limited to the aforementioned variants of SARS-CoV-2 and encompasses variants identified in the future. The one or more coronaviruses that cause the common cold may include but are not limited to strains 229E (alpha coronavirus), NL63 (alpha coronavirus), OC43 (beta coronavirus), HKU1 (beta coronavirus).

Additionally, other coronaviruses may be used for determining conserved epitopes (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID”, ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, Increased risk of particular conditions such as multisystem inflammatory syndrome or long-haul COVID or Increased affinity for particular demographic or clinical groups, such as children or immunocompromised individuals. Once validated, variants of interest are renamed “variant of concern” by monitoring organizations, such as the CDC.

The present invention may feature a multi-epitope, pan-coronavirus vaccine composition, the composition comprising at least two of: a) one or more conserved coronavirus B-cell target epitopes selected from SEQ ID NO: 23-31, or a combination thereof; b) one or more conserved coronavirus CD4+ T cell target epitopes selected from SEQ ID NO: 17-22, or a combination thereof; or c) one or more conserved coronavirus CD8+ T cell target epitopes selected from SEQ ID NO: 1-16, or a combination thereof. In some embodiments, at least one epitope is derived from a non-spike protein, wherein the composition induces immunity to only the epitopes

TABLE 1
Shows non-limiting examples of conserved coronavirus epitopes.

| | | | SEQ |
|---|---|---|---|
| Target | | | ID |
| Epitope | Protein | Epitope Sequence | NO: |
| CD8⁺ T-cells | ORF1ab₂₂₁₀₋₂₂₁₈(nsp3) | CLEASFNYL | 1 |
| CD8⁺ T-cells | ORF1ab₃₀₁₃₋₃₀₂₁(nsp4) | SLPGVFCGV | 2 |
| CD8⁺ T-cells | ORF1ab₄₂₈₃₋₄₂₉₁₀(nsp10) | ALNTLVKQL | 3 |
| CD8⁺ T-cells | ORF1ab₆₇₄₉₋₆₇₅₇(nsp15) | LLLDDFVEI | 4 |
| CD8⁺ T-cells | S₉₅₈₋₉₆₆ | ALNTLVKQL | 5 |
| CD8⁺ T-cells | S₁₀₀₀₋₁₀₀₈ | RLQSLQTYV | 6 |
| CD8⁺ T-cells | S(1220-1228) | FIAGLIAIV | 7 |
| CD8⁺ T-cells | E(20-28) | FLAFVVFLL | 8 |
| CD8⁺ T-cells | E(26-34) | FLLVTLAIL | 9 |
| CD8⁺ T-cells | M(52-60) | IFLWLLWPV | 10 |
| CD8⁺ T-cells | M(89-97) | GLMWLSYFI | 11 |
| CD8⁺ T-cells | ORF6(4-11) | IHLVDFQVT | 12 |
| CD8⁺ T-cells | ORF7b(26-34) | IIFWFSLEL | 13 |
| CD8⁺ T-cells | ORF8s(73-81) | YIDIGNYTV | 14 |
| CD8⁺ T-cells | ORF10(3-11) | YINVFAFPF | 15 |
| CD8⁺ T-cells | ORF10(5-12) | NVFAFPFTI | 16 |
| CD4+ T-cells | ORF1ab(1350-1365) | KSAFYILPSIISNEK | 17 |
| | (nsp3) | | |
| CD4+ T-cells | S(1-13) | MFVFLVLLPLVSS | 18 |
| CD4+ T-cells | M(176-190) | LSYYKLGASQRVAGD | 19 |
| CD4+ T-cells | ORF6(12-26) | MKFLVFLGIITTVAA | 20 |
| CD4+ T-cells | ORF8b(1-15) | AEILLIIMRTFKVSI | 21 |
| CD4+ T-cells | N(388-403) | KQQTVTLLPAADLDDF | 22 |
| B-cells | S(13-37) | SQCVNLTTRTQLPPAYTNSFTRGVY | 23 |
| B-cells | S(287-317) | DAVDCALDPLSETKCTLKSFTVEKGIYQTSN | 24 |
| B-cells | S(338-363) | FGEVFNATRFASVYAWNRKRISNCVA | 25 |
| B-cells | S(369-393) | YNSASFSTFKCYGVSPTKLNDLCFT | 26 |
| B-cells | S(471-501) | EIYQAGSTPCNGVEGFNCYFPLQSYGFQPTN | 27 |
| B-cells | S(524-558) | VCGPKKSTNLVKNKCVNFNFNGLTGTGVLTESNKK | 28 |
| B-cells | S(565-598) | FGRDIADTTDAVRDPQTLEILDITPCSFGGVSVI | 29 |
| B-cells | S(601-628) | GTNTSNQVAVLYQDVNCTEVPVAIHADQ | 30 |
| B-cells | S(614-640) | DVNCTEVPVAIHADQLTPTWRVYSTGS | 31 |

In certain embodiments, the composition comprises 1-16 CD8+ T cell target epitopes. In certain embodiments, the composition comprises 2-16 CD8+ T cell target epitopes. In certain embodiments, the composition comprises 2-10 CD8+ T cell target epitopes. In certain embodiments, the composition comprises 2-15 CD8+ T cell target epitopes. In certain embodiments, the composition comprises 2-5 CD8⁺ T cell target epitopes. In certain embodiments, the composition comprises 5-10 CD8⁺ T cell target epitopes. In certain embodiments, the composition comprises 5-15 CD8⁺ T cell target epitopes.

In certain embodiments, the composition comprises 1-6 CD4⁺ T cell target epitopes. In certain embodiments, the composition comprises 2-6 CD4⁺ T cell target epitopes. In certain embodiments, the composition comprises 3-6 CD4⁺ T cell target epitopes. In certain embodiments, the composition comprises 4-6 CD4⁺ T cell target epitopes. In certain embodiments, the composition comprises 5-6 CD4⁺ T cell target epitopes. In certain embodiments, the composition comprises 2-5 CD4⁺ T cell target epitopes. In certain embodiments, the composition comprises 3-5 CD4⁺ T cell target epitopes.

In certain embodiments, the composition comprises 1-9 of the aforementioned B cell target epitopes. In certain embodiments, the composition comprises 2-9 B cell target epitopes. In certain embodiments, the composition comprises 2-5 B cell target epitopes. In certain embodiments, the composition comprises 5-9 B cell target epitopes.

The epitopes may be each separated by a linker. In certain embodiments, the linker allows for an enzyme to cleave between the target epitopes. The present invention is not limited to particular linkers or particular lengths of linkers. As an example, in certain embodiments, one or more epitopes may be separated by a linker 2 amino acids in length. In certain embodiments, one or more epitopes may be separated by a linker 3 amino acids in length. In certain embodiments, one or more epitopes may be separated by a linker 4 amino acids in length. In certain embodiments, one or more epitopes may be separated by a linker 5 amino acids in length. In certain embodiments, one or more epitopes may be separated by a linker 6 amino acids in length. In certain embodiments, one or more epitopes may be separated by a linker 7 amino acids in length. In certain embodiments, one or more epitopes may be separated by a linker 8 amino acids in length. In certain embodiments, one or more epitopes may be separated by a linker 9 amino acids in length. In certain embodiments, one or more epitopes may be separated by a linker 10 amino acids in length. In certain embodiments, one or more epitopes may be separated by a linker from 2 to 10 amino acids in length.

Linkers are well known to one of ordinary skill in the art. Non-limiting examples of linkers include AAY, KK, and GPGPG. For example, in certain embodiments, one or more CD8+ T cell epitopes are separated by AAY. In some embodiments, one or more CD4+ T cell epitopes are separated by GPGPG. In certain embodiments, one or more B cell epitopes are separated by KK. In certain embodiments, KK is a linker between a CD4+ T cell epitope and a B cell epitope. In certain embodiments, KK is a linker between a CD8+ T cell epitope and a B cell epitope. In certain embodiments, KK is a linker between a CD8+ T cell epitope and a CD4+ T cell epitope. In certain embodiments, AAY is a linker between a CD4 T cell epitope and a B cell epitope. In certain embodiments, AAY is a linker between a CD8+ T cell epitope and a B cell epitope. In certain embodiments, AAY is a linker between a CD8+ T cell epitope and a CD4+ T cell epitope. In certain embodiments, GPGPG is a linker between a CD4+ T cell epitope and a B cell epitope. In certain embodiments, GPGPG is a linker between a CD8+ T cell epitope and a B cell epitope. In certain embodiments, GPGPG is a linker between a CD8+ T cell epitope and a CD4+ T cell epitope.

The target epitopes may be derived from structural proteins, non-structural proteins, or a combination thereof. For example, structural proteins may include spike proteins(S), envelope proteins (E), membrane proteins (M), or nucleoproteins (N).

In some embodiments, the target epitopes are derived from at least one SARS-CoV-2 protein. The SARS-CoV-2 proteins may include ORF1ab protein, Spike glycoprotein, ORF3a protein, Envelope protein, Membrane glycoprotein, ORF6 protein, ORF7a protein, ORF7b protein, ORF8 protein, Nucleocapsid protein, and ORF10 protein. The ORF1ab protein provides nonstructural proteins (Nsp) such as Nsp1, Nsp2, Nsp3 (Papain-like protease), Nsp4, Nsp5 (3C-like protease), Nsp6, Nsp7, Nsp8, Nsp9, Nsp10, Nsp11, Nsp12 (RNA polymerase), Nsp13 (5′ RNA triphosphatase enzyme), Nsp14 (guanosineN7-methyltransferase), Nsp15 (endoribonuclease), and Nsp16 (2′-O-ribose-methyltransferase).

In some embodiments, the antigen or antigens (e.g., epitopes) are operatively linked to a promoter. In certain embodiments, the antigen or antigens (e.g., epitopes) are operatively linked to a generic promoter. For example, in certain embodiments, the antigen or antigens (e.g., epitopes) are operatively linked to a CMV promoter. In certain embodiments, the antigen or antigens (e.g., epitopes) are operatively linked to a CAG, EFIA, EFS, CBh, SFFV, MSCV, mPGK, hPGK, SV40, UBC, or other appropriate promoter.

In some embodiments, the antigen or antigens (e.g., epitopes) are operatively linked to a tissue-specific promoter (e.g., a lung-specific promoter). For example, the antigen or antigens (e.g., epitopes) may be operatively linked to a SpB promoter or a CD144 promoter.

Table 2 shows non-limiting examples of a portion of a promoter that may be used in accordance with the present invention.

| | | SEQ |
|---|---|---|
| | Sequence | ID NO: |
| CMV | TAGTTATTAATAGTAATCAATTACGGGGTCATTAGTTCATAGCCCATATATGGAGTTC | 32 |
| | CGCGTTACATAACTTACGGTAAATGGCCCGCCTGGCTGACCGCCCAACGACCCC | |
| | CGCCCATTGACGTCAATAATGACGTATGTTCCCATAGTAACGCCAATAGGGACTTT | |
| | CCATTGACGTCAATGGGTGGAGTATTTACGGTAAACTGCCCACTTGGCAGTACAT | |
| | CAAGTGTATCATATGCCAAGTACGCCCCCTATTGACGTCAATGACGGTAAATGGCC | |
| | CGCCTGGCATTATGCCCAGTACATGACCTTATGGGACTTTCCTACTTGGCAGTACA | |
| | TCTACGTATTAGTCATCGCTATTACCATGGTGATGCGGTTTTGGCAGTACATCAATG | |
| | GGCGTGGATAGCGGTTTGACTCACGGGGATTTCCAAGTCTCCACCCCATTGACG | |
| | TCAATGGGAGTTTGTTTTGGCACCAAAATCAACGGGACTTTCCAAAATGTCGTAA | |
| | CAACTCCGCCCCATTGACGCAAATGGGCGGTAGGCGTGTACGGTGGGAGGTCTA | |
| | TATAAGCAGAGCTGGTTTAGTGAACCGTCAGATC | |
| CAG | CTCGACATTGATTATTGACTAGTTATTAATAGTAATCAATTACGGGGTCATTAGTTCA | 33 |
| | TAGCCCATATATGGAGTTCCGCGTTACATAACTTACGGTAAATGGCCCGCCTGGCT | |
| | GACCGCCCAACGACCCCCGCCCATTGACGTCAATAATGACGTATGTTCCCATAGT | |
| | AACGCCAATAGGGACTTTCCATTGACGTCAATGGGTGGAGTATTTACGGTAAACT | |
| | GCCCACTTGGCAGTACATCAAGTGTATCATATGCCAAGTACGCCCCCTATTGACGT | |
| | CAATGACGGTAAATGGCCCGCCTGGCATTATGCCCAGTACATGACCTTATGGGAC | |
| | TTTCCTACTTGGCAGTACATCTACGTATTAGTCATCGCTATTACCATGGTCGAGGT | |
| | GAGCCCCACGTTCTGCTTCACTCTCCCCATCTCCCCCCCCTCCCCACCCCCAAT | |
| | TTTGTATTTATTTATTTTTTAATTATTTTGTGCAGCGATGGGGGCGGGGGGGGGGGG | |
| | GGGGGCGCGCGCCAGGCGGGGCGGGGCGGGGCGAGGGGCGGGGCGGGGCG | |
| | AGGCGGAGAGGTGCGGCGGCAGCCAATCAGAGCGGCGCGCTCCGAAAGTTTC | |
| | CTTTTATGGCGAGGCGGCGGCGGCGGCGGCCCTATAAAAAGCGAAGCGCGCGG | |
| | CGGGCGGGAGTCGCTGCGCGCTGCCTTCGCCCCGTGCCCCGCTCCGCCGCCG | |
| | CCTCGCGCCGCCCGCCCCGGCTCTGACTGACCGCGTTACTCCCACAGGTGAGC | |
| | GGGCGGGACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAGGGCCCT | |
| | TTGTGCGGGGGGAGCGGCTCGGGGGGTGCGTGCGTGTGTGTGTGCGTGGGGA | |
| | GCGCCGCGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGGCCGGGG | |
| | GCGGTGCCCCGCGGTGCGGGGGGGGCTGCGAGGGGAACAAAGGCTGCGTGC | |
| | GGGGTGTGTGCGTGGGGGGGTGAGCAGGGGGTGTGGGCGCGTCGGTCGGGC | |
| | TGCAACCCCCCCTGCACCCCCCTCCCCGAGTTGCTGAGCACGGCCCGGCTTCG | |
| | GGTGCGGGGCTCCGTACGGGGCGTGGCGCGGGGCTCGCCGTGCCGGGCGGG | |
| | GGGTGGCGGCAGGTGGGGGTGCCGGGCGGGGCGGGGCCGCCTCGGGCCGG | |
| | GGAGGGCTCGGGGGAGGGGCGCGGCGGCCCCCGGAGCGCCGGCGGCTGTCG | |
| | AGGCGCGGCGAGCCGCAGCCATTGCCTTTTATGGTAATCGTGCGAGAGGGCGC | |
| | AGGGACTTCCTTTGTCCCAAATCTGTGCGGAGCCGAAATCTGGGAGGCGCCGCC | |
| | GCACCCCCTCTAGCGGGCGCGGGGCGAAGCGGTGCGGCGCCGGCAGGAAGGA | |
| | AATGGGGGGGGAGGGCCTTCGTGCGTCGCCGCGCCGCCGTCCCCTTCTCCCTC | |
| | TCCAGCCTCGGGGCTGTCCGCGGGGGGACGGCTGCCTTCGGGGGGGACGGG | |
| | GCAGGGCGGGGTTCGGCTTCTGGCGTGTGACCGGCGGCTCTAGAGCCTCTGCT | |
| | AACCATGTTCATGCCTTCTTCTTTTTCCTACAGCTCCTGGGCAACGTGCTGGTTAT | |
| | TGTGCTGTCTCATCATTTTGGCAAAGAATTG | |
| SP-B | GTATAGGGCTGTCTGGGAGCCACTCCAGGGCCACAGAAATCTTGTCTCTGACTC | 34 |
| | AGGGTATTTTGTTTTCTGTTTTGTGTAAATGCTCTTCTGACTAATGCAAACCATGTG | |
| | TCCATAGAACCAGAAGATTTTTCCAGGGGAAAAGGTAAGGAGGTGGTGAGAGTG | |
| | TCCTGGGTCTGCCCTTCCAGGGCTTGCCCTGGGTTAAGAGCCAGGCAGGAAGC | |
| | TCTCAAGAGCATTGCTCAAGAGTAGAGGGGGCCTGGGAGGCCCAGGGAGGGGA | |
| | TGGGAGGGGAACACCCAGGCTGCCCCCAACCAGATGCCCTCCACCCTCCTCAA | |
| | CCTCCCTCCCACGGCCTGGAGAGGTGGGACCAGGTATGGAGGCTTGAGAGCCC | |
| | CTGGTTGGAGGAAGCCACAAGTCCAGGAACATGGGAGTCTGGGCAGGGGGCAA | |
| | AGGAGGCAGGAACAGGCCATCAGCCAGGACAGGTGGTAAGGCAGGCAGGAGT | |
| | GTTCCTGCTGGGAAAAGGTGGGATCAAGCACCTGGAGGGCTCTTCAGAGCAAA | |
| | GACAAACACTGAGGTCGCTGCCACTCCTACAGAGCCCCCACGCCCCGCCCAGC | |
| | TATAAGGGGCCATGCACCAAGCAGGGTACCCAGGCTGCAGAGGTGCC | |
| CD144 | CATCCATGCCCATGGCCTCAGATGCCAGCCATAAGCTGTTGGGTTCCAAACCTCG | 35 |
| | ACTCCAGGCTGGACTCACCCCTGTCTCCCCCACCAGCCTGACACCTCCACCTGG | |
| | GTATCTAACGAGCATCTCAAACTCAACCTGCCTGAGACAGAGGAATCACTATCCC | |
| | CTCCTCCTCCAAAAATATCCTTCCATCACACTCCCCATCTTGTGCTCTGATTTACTA | |
| | AACGGCCCTGGGCCCTCTCTTTCTCAGGGTCTCTGCTTGCCCAGCTATATAATAA | |
| | AACAAGTTTGGGACTTCCCAACCATTCACCCATGGAAAAACAGAAGCAACTCTTC | |
| | AAAGGACAGATTCCCAGGATCTGCCCTGGGAGATTCCAAATCAGTTGATCTGGG | |
| | GTGAGCCCAGTCCTCTGTAGTTTTTAGAAGCTCCTCCTATGTCTCTCCTGGTCAG | |
| | CAGAATCTTGGCCCCTCCCTTCCCCCCAGCCTCTTGGTTCTTCTGGGCTCTGATC | |
| | CAGCCTCAGCGTCACTGTCTTCCACGCCCCTCTTTGATTCTCGTTTATGTCAAAA | |
| | GCCTTGTGAGGATGAGGCTGTGATTATCCCCATTTTACAGATGAGGAAACTGTGG | |
| | CTCCAGGATGACACAACTGGCCAGAGGTCACATCAGAAGCAGAGCTGGGTCACT | |
| | TGACTCCACCCAATATCCCTAAATGCAAACATCCCCTACAGACCGAGGCTGGCAC | |
| | CTTAGAGCTGGAGTCCATGCCCGCTCTGACCAGGAGAAGCCAACCTGGTCCTCC | |
| | AGAGCCAAGAGCTTCTGTCCCTTTCCCATCTCCTGAAGCCTCCCTGTCACCTTTA | |
| | AAGTCCATTCCCACAAAGACATCATGGGATCACCACAGAAAATCAAGCTCTGGGG | |
| | CTAGGCTGACCCCAGCTAGATTTTTGGCTCTTTTATACCCCAGCTGGGTGGACAA | |
| | GCACCTTAAACCCGCTGAGCCTCAGCTTCCCGGGCTATAAAATGGGGGTGATGA | |
| | CACCTGCCTGTAGCATTCCAAGGAGGGTTAAATGTGATGCTGCAGCCAAGGGTC | |
| | CCCACAGCCAGGCTCTTTGCAGGTGCTGGGTTCAGAGTCCCAGAGCTGAGGCC | |
| | GGGAGTAGGGGTTCAAGTGGGGTGCCCCAGGCAGGGTCCAGTGCCAGCCCTCT | |
| | GTGGAGACAGCCATCCGGGGCCGAGGCAGCCGCCCACCGCAGGGCCTGCCTA | |
| | TCTGCAGCCAGCCCAGCCCTCACAAAGGAACAATAACAGGAAACCATCCCAGGG | |
| | GGAAGTGGGCCAGGGCCAGCTGGAAAACCTGAAGGGGAGGCAGCCAGGCCTC | |
| | CCTCGCCAGCGGGGTGTGGCTCCCCTCCAAAGACGGTCGGCTGACAGGCTCCA | |
| | CAGAGCTCCACTCACGCTCAGCCCTGGACGGACAGGCAGTCCAACGGAACAGA | |
| | AACATCCCTCAGCCCACAGGCACGGTGAGTGGGGGCTCCCACACTCCCCTCCA | |
| | CCCCAAACCCGCCACCCTGCGCCCAAGATGGGAGGGTCCTCAGCTTCCCCATCT | |
| | GTAGAATGGGCATCGTCCCACTCCCATGACAGAGAGGCTCC | |

In certain embodiments, the vaccine composition comprises a molecular adjuvant and/or one or more T Cell enhancement compositions. The adjuvant and/or enhancement compositions may help improve the immunogenicity and/or long-term memory of the vaccine composition. Non-limiting examples of molecular adjuvants include CpG, such as a CpG polymer, and flagellin.

In some embodiments, the vaccine composition comprises a T cell attracting chemokine. The T cell attracting chemokine helps pull the T cells from the circulation to the appropriate tissues, e.g., the lungs, heart, kidney, and brain. Non-limiting examples of T cell attracting chemokines include CCL5, CXCL9, CXCL10, CXCL11, CCL25, CCL28, CXCL14, CXCL17, or a combination thereof.

In some embodiments, the vaccine composition comprises a composition that promotes T cell proliferation. Non-limiting examples of compositions that promote T cell proliferation include IL-7, IL-15, IL-2, or a combination thereof.

In some embodiments, the vaccine composition comprises a composition that promotes T cell homing in the lungs. Non-limiting examples of compositions that promote T cell homing include CCL25, CCL28, CXCL14, CXCL17 or a combination thereof.

Table 3: example of vaccine compositions described herein. The present invention is not limited to the examples in Table 3.

| Vaccine | | SEQ ID |
|---|---|---|
| Candidate | Sequence | NO: |
| 1 | CCTGCAGGCAGCTGCGCGCTCGCTCGCTCACTGAGGCCGCCCGGGC | 36 |
| | AAAGCCCGGGGGTCGGGCGACCTTTGGTCGCCCGGCCTCAGTGAGC | |
| | GAGCGAGCGCGCAGAGAGGGAGTGGCCAACTCCATCACTAGGGGTT | |
| | CCTTCTAGACAACTTTGTATAGAAAAGTTGCTCGACATTGATTATTGACT | |
| | AGTTATTAATAGTAATCAATTACGGGGTCATTAGTTCATAGCCCATATATG | |
| | GAGTTCCGCGTTACATAACTTACGGTAAATGGCCCGCCTGGCTGACCG | |
| | CCCAACGACCCCCGCCCATTGACGTCAATAATGACGTATGTTCCCATAG | |
| | TAACGCCAATAGGGACTTTCCATTGACGTCAATGGGTGGAGTATTTACG | |
| | GTAAACTGCCCACTTGGCAGTACATCAAGTGTATCATATGCCAAGTACG | |
| | CCCCCTATTGACGTCAATGACGGTAAATGGCCCGCCTGGCATTATGCC | |
| | CAGTACATGACCTTATGGGACTTTCCTACTTGGCAGTACATCTACGTATT | |
| | AGTCATCGCTATTACCATGGTCGAGGTGAGCCCCACGTTCTGCTTCAC | |
| | TCTCCCCATCTCCCCCCCCTCCCCACCCCCAATTTTGTATTTATTTATTT | |
| | TTTAATTATTTTGTGCAGCATGGGGGCGGGGGGGGGGGGGGGGCG | |
| | CGCGCCAGGCGGGGCGGGGCGGGGCGAGGGGCGGGGCGGGGCGA | |
| | GGCGGAGAGGTGCGGGGGCAGCCAATCAGAGCGGCGCGCTCCGAAA | |
| | GTTTCCTTTTATGGCGAGGGGGGGGCGGGGGGGGCCCTATAAAAAGC | |
| | GAAGCGCGGGGGGGGGGGGAGTCGCTGCGCGCTGCCTTCGCCCCG | |
| | TGCCCCGCTCCGCCGCCGCCTCGCGCCGCCCGCCCCGGCTCTGACT | |
| | GACCGCGTTACTCCCACAGGTGAGCGGGGGGGACGGCCCTTCTCCT | |
| | CCGGGCTGTAATTAGCGCTTGGTTTAATGACGGCTTGTTTCTTTTCTGT | |
| | GGCTGCGTGAAAGCCTTGAGGGGCTCCGGGAGGGCCCTTTGTGCGG | |
| | GGGGAGCGGCTCGGGGGGTGCGTGCGTGTGTGTGTGCGTGGGGAG | |
| | CGCCGCGTGCGGCTCCGCGCTGCCCGGGGGCTGTGAGCGCTGCGG | |
| | GCGCGGCCGGGGCTTTGTGCGCTCCGCAGTGTGCGCGAGGGGAG | |
| | CGCGGCCGGGGGGGGTGCCCCGCGGTGCGGGGGGGGCTGCGAGG | |
| | GGAACAAAGGCTGCGTGCGGGGTGTGTGCGTGGGGGGGTGAGCAG | |
| | GGGGTGTGGGGGCGTCGGTCGGGCTGCAACCCCCCCTGCACCCCCC | |
| | TCCCCGAGTTGCTGAGCACGGCCCGGCTTCGGGTGGGGGGCTCCGT | |
| | ACGGGGCGTGGCGCGGGGCTCGCCGTGCCGGGCGGGGGGTGGCG | |
| | GCAGGTGGGGGTGCCGGGCGGGGCGGGGCCGCCTCGGGCCGGGG | |
| | AGGGCTCGGGGGAGGGGCGCGGCGGCCCCCGGAGCGCCGGCGGC | |
| | TGTCGAGGCGCGGGGAGCCGCAGCCATTGCCTTTTATGGTAATCGTG | |
| | CGAGAGGGCGCAGGGACTTCCTTTGTCCCAAATCTGTGCGGAGCCGA | |
| | AATCTGGGAGGCGCCGCCGCACCCCCTCTAGGGGGGGGGGGGCGAA | |
| | GCGGTGGGGGGCCGGCAGGAAGGAAATGGGGGGGGAGGGCCTTCG | |
| | TGCGTCGCCGCGCCGCCGTCCCCTTCTCCCTCTCCAGCCTCGGGGC | |
| | TGTCCGGGGGGGGACGGCTGCCTTCGGGGGGGACGGGGCAGGGCG | |
| | GGGTTCGGCTTCTGGCGTGTGACCGGGGGCTCTAGAGCCTCTGCTAA | |
| | CCATGTTCATGCCTTCTTCTTTTTCCTACAGCTCCTGGGCAACGTGCTG | |
| | GTTATTGTGCTGTCTCATCATTTTGGCAAAGAATTGCAAGTTTGTACAAA | |
| | AAAGCAGGCTGCCACCATGTTCGTGTTTCTGGTCCTGCTGCCTCTGGT | |
| | GAGCTCCCTGCTGCTGGACGACTTTGTGGAAATTGCTGCTTATTACCT | |
| | CGCTTCCGGCGGCCAGCCAATCGCCGCCTATGCCCTGAACACCCTGG | |
| | TGAAGCAGCTGGCTGCATATTTTCTGGCTTTCGTGGTGTTTCTGCTGG | |
| | CCGCCTACTTCATCGCCGGGCTGATCGCTATCGTGGCCGCCTATTTTC | |
| | TGCTGGTGACACTGGCCATTCTGGCCGCCTACTGCCTGGAGGCCTCC | |
| | TTCAACTACCTGGCCGCCTACTACATCGACATCGGCAACTACACCGTG | |
| | GCCGCCTATATCATTTTCTGGTTCTCACTGGAGCTGGCCGCTTACTCCC | |
| | TGCCCGGCGTGTTCTGTGGAGTGGCCGCTTACAACGTGTTTGCCTTC | |
| | CCCTTCACCATCGCAGCCTACGGTCTGATGTGGCTGTCATACTTCATC | |
| | GCAGCCTATCATCTGGTGGACTTCCAGGTGACTATCGCAGCCTACATC | |
| | TTCCTGTGGCTGCTGTGGCCCGTGGCCGCATACTACATTAATGTGTTC | |
| | GCCTTTCCTTTTGCCGCCTACAGACTGCAGAGCCTGCAGACCTACGTC | |
| | GGCCCTGGCCCTGGGAAGTCCGCCTTCTACATCCTGCCCAGTATCATT | |
| | TCCAACGAGAAGGGGCCAGGACCGGGAATGTTCGTGTTCCTGGTGCT | |
| | GTTGCCACTGGTGAGTAGCGGCCCCGGACCAGGCCTGAGCTACTATA | |
| | AGCTGGGCGCCTCCCAGGGGGTGGCTGGCGACGGCCCCGGACCAG | |
| | GCGCTGAGATCCTGCTGATTATCATGAGGACCTTCAAGGTGTCCATCG | |
| | GGCCTGGTCCTGGAATGAAGTTCCTGGTGTTCCTGGGCATCATCACTA | |
| | CCGTGGCCGCCGGGCCTGGGCCAGGCAAGCAGCAGACCGTGACCCT | |
| | GCTGCCAGCTGCCGATCTGGATGATTTCAAGAAGGACGCCGTGGACT | |
| | GCGCCCTGGATCCCCTGTCCGAAACTAAGTGTACCCTGAAGTCTTTCA | |
| | CAGTGGAGAAGGGCATCTACCAGACAAGCAACAAGAAGGGCACCAAT | |
| | ACTTCTAACCAGGTCGCCGTGCTGTACCAGGACGTGAACTGCACTGAA | |
| | GTCCCCGTGGCCATTCACGCTGACCAGGACGTGAACTGCACAGAGGT | |
| | GCCAGTGGCCATCCACGCCGACCAACTGACCCCCACCTGGCGAGTGT | |
| | ACTCCACTGGCTCCAAGAAATCCCAGTGCGTGAATTTGACTACCAGAA | |
| | CACAGCTGCCCCCCGCTTACACCAACAGTTTCACAAGAGGGGTCTACA | |
| | AGAAGGTGTGCGGACCAAAGAAAAGCACCAACCTGGTGAAAAACAAG | |
| | TGCGTGAATTTCAACTTCAATGGACTGACCGGCACGGGAGTGCTGAC | |
| | CGAGAGCAATAAGAAGAAAAAGTTTGGACGCGACATCGCCGATACAAC | |
| | CGATGCCGTGAGGGACCCACAGACACTGGAGATCCTCGACATCACCC | |
| | CATGTAGTTTTGGCGGAGTGTCAGTGATCAAGAAGGAGATTTACCAGG | |
| | CCGGCAGCACCCCATGCAACGGCGTGGAAGGCTTTAATTGCTACTTTC | |
| | CACTGCAGTCCTATGGATTCCAGCCCACCAACAAAAAGTATAATTCTGC | |
| | CTCCTTTAGCACTTTTAAGTGCTATGGCGTGAGCCCCACAAAACTGAAC | |
| | GATCTGTGCTTCACAAAGAAGTTTGGCGAGGTGTTT | |

### Large Sequence Vaccines

The present invention features a pan-coronavirus vaccine composition. The composition comprising one or more conserved large sequences, each of the one or more conserved large sequences comprise at least one of: one or more conserved coronavirus B-cell target epitopes, one or more conserved coronavirus CD4+ T cell target epitopes, and/or one or more conserved coronavirus CD8+ T cell target epitopes.

The present invention may also feature a pan-coronavirus vaccine composition. The composition comprising two or more conserved large sequences, each of the two or more conserved large sequences comprise at least one of: one or more conserved coronavirus B-cell target epitopes, one or more conserved coronavirus CD4+ T cell target epitopes, and/or one or more conserved coronavirus CD8+ T cell target epitopes. In some embodiments, at least one large sequence is derived from a non-spike protein.

As used herein, the term “conserved” refers to a large sequence that is among the most highly conserved large sequences identified in a sequence alignment and analysis. For example, the conserved large sequences may be the 2 most highly conserved sequences identified. In some embodiments, the conserved large sequences may be the 3 most highly conserved sequences identified. In some embodiments, the conserved large sequences may be the 4 most highly conserved sequences identified. In some embodiments, the conserved large sequences may be the 5 most highly conserved sequences identified. In some embodiments, the conserved large sequences may be the 6 most highly conserved sequences identified. In some embodiments, the conserved large sequences may be the 7 most highly conserved sequences identified. In some embodiments, the conserved large sequences may be the 8 most highly conserved sequences identified. In some embodiments, the conserved large sequences may be the 9 most highly conserved sequences identified. In some embodiments, the conserved large sequences may be the 10 most highly conserved sequences identified. In some embodiments, the conserved large sequences may be the 15 most highly conserved sequences identified. In some embodiments, the conserved large sequences may be the 20 most highly conserved sequences identified. In some embodiments, the conserved large sequences may be the 25 most highly conserved sequences identified. In some embodiments, the conserved large sequences may be the 30 most highly conserved sequences identified. In some embodiments, the conserved large sequences may be the 40 most highly conserved sequences identified. In some embodiments, the conserved large sequences may be the 50 most highly conserved sequences identified. In some embodiments, the conserved sequences may be the 50% most highly conserved large sequences identified. In some embodiments, the conserved large sequences may be the 60% most highly conserved sequences identified. In some embodiments, the large conserved sequences may be the 70% most highly conserved sequences identified. In some embodiments, the conserved large sequences may be the 80% most highly conserved sequences identified. In some embodiments, the conserved large sequences may be the 90% most highly conserved sequences identified. In some embodiments, the conserved large sequences may be the 95% most highly conserved sequences identified. In some embodiments, the conserved large sequences may be the 99% most highly conserved sequences identified. The present invention is not limited to the aforementioned thresholds.

In some embodiments, the non-spike protein is ORF1ab protein, ORF3a protein, Envelope protein, Membrane glycoprotein, ORF6 protein, ORF7a protein, ORF7b protein, ORF8 protein, Nucleocapsid protein and ORF10 protein.

The present invention may further feature a pan-coronavirus vaccine composition, the composition comprising one or more large sequences, wherein each of the one or more large sequences comprise conserved regions of a coronavirus.

In some embodiments, the portion of the coronavirus spike(S) protein is highly conserved among human and animal coronaviruses. The portion of the coronavirus spike(S) protein may be derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold.

In some embodiments, the one or more large sequences are highly conserved among human and animal coronaviruses. In some embodiments, the one or more large sequences are derived from at least one of SARS-CoV-2 proteins. In some embodiments, the one or more large sequences are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. The conserved large sequences may be selected from Variants of Concern or Variants of Interest.

In some embodiments, the one or more SARS-CoV-2 human strains or variants in current circulation are selected from: variant B.1.177; variant B.1.160, variant B.1.1.7; variant B.1.351; variant P.1; variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; variant B.1.1.529-Omicron (BA.1); variant B.1.1.529-Omicron (BA.2); and variant B.1.617.2-Delta). In some embodiments, the one or more coronaviruses that cause the common cold are selected from: 229E alpha coronavirus, NL63 alpha coronavirus, OC43 beta coronavirus, and HKU1 beta coronavirus.

In some embodiments, the one or more large sequences are derived from a whole protein sequence expressed by SARS-CoV-2. In some embodiments, the one or more large sequences are derived from a partial protein sequence expressed by SARS-CoV-2. In other embodiments, the one or more large conserved sequences is derived from a full-length spike glycoprotein. In some embodiments, the one or more large conserved sequences is derived from a partial spike glycoprotein. In some embodiments the spike(S) protein comprises at least one proline substitution, or at least two proline substitution, or at least, four proline substitution, or at least six proline substitution. The spike(S) protein may comprise two consecutive proline substitutions at amino acid positions 986 and 987. The proline substitutions may comprise K986P and V987P mutations. In further embodiments, the spike(S) protein is receptor-binding domain (RBD). In some embodiments, the RBD comprises a trimerized SARS-CoV-2 receptor-binding domain (RBD).

Table 4: Shows non-limiting examples of a portion of a coronavirus spike(S) protein that may be used in accordance with the present invention.

| | | SEQ |
|---|---|---|
| | | ID |
| | Sequence | NO: |
| spike | MFVFLVLLPLVSSQCVNLTTRTQLPPAYTNSFTRGVYYPDKVFRSSVLHSTQDL | 37 |
| glycoprotein | FLPFFSNVTWFHAIHVSGTNGTKRFDNPVLPFNDGVYFASTEKSNIIRGWIFGT | |
| with two | TLDSKTQSLLIVNNATNVVIKVCEFQFCNDPFLGVYYHKNNKSWMESEFRVYS | |
| proline | SANNCTFEYVSQPFLMDLEGKQGNFKNLREFVFKNIDGYFKIYSKHTPINLVRD | |
| substitutions | LPQGFSALEPLVDLPIGINITRFQTLLALHRSYLTPGDSSSGWTAGAAAYYVGYL | |
| (K986P, | QPRTFLLKYNENGTITDAVDCALDPLSETKCTLKSFTVEKGIYQTSNFRVQPTE | |
| V987P) | SIVRFPNITNLCPFGEVFNATRFASVYAWNRKRISNCVADYSVLYNSASFSTFKC | |
| | YGVSPTKLNDLCFTNVYADSFVIRGDEVRQIAPGQTGKIADYNYKLPDDFTGCV | |
| | IAWNSNNLDSKVGGNYNYLYRLFRKSNLKPFERDISTEIYQAGSTPCNGVEGF | |
| | NCYFPLQSYGFQPTNGVGYQPYRVVVLSFELLHAPATVCGPKKSTNLVKNKCV | |
| | NFNFNGLTGTGVLTESNKKFLPFQQFGRDIADTTDAVRDPQTLEILDITPCSFG | |
| | GVSVITPGTNTSNQVAVLYQDVNCTEVPVAIHADQLTPTWRVYSTGSNVFQTR | |
| | AGCLIGAEHVNNSYECDIPIGAGICASYQTQTNSPRRARSVASQSIIAYTMSLGA | |
| | ENSVAYSNNSIAIPTNFTISVTTEILPVSMTKTSVDCTMYICGDSTECSNLLLQYG | |
| | SFCTQLNRALTGIAVEQDKNTQEVFAQVKQIYKTPPIKDFGGFNFSQILPDPSKP | |
| | SKRSFIEDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLYENQKL | |
| | IANQFNSAIGKIQDSLSSTASALGKLQDVVNQNAQALNTLVKQLSSNFGAISSVL | |
| | NDILSRLDPPEAEVQIDRLITGRLQSLQTYVTQQLIRAAEIRASANLAATKMSEC | |
| | VLGQSKRVDFCGKGYHLMSFPQSAPHGVVFLHVTYVPAQEKNFTTAPAICHDG | |
| | KAHFPREGVFVSNGTHWFVTQRNFYEPQIITTDNTFVSGNCDVVIGIVNNTVYD | |
| | PLQPELDSFKEELDKYFKNHTSPDVDLGDISGINASVVNIQKEIDRLNEVAKNLN | |
| | ESLIDLQELGKYEQYIKWPWYIWLGFIAGLIAIVMVTIMLCCMTSCCSCLKGCCS | |
| | CGSCCKFDEDDSEPVLKGVKLHYT | |
| spike | MFVFLVLLPLVSSQCVNLTTRTQLPPAYTNSFTRGVYYPDKVFRSSVLHSTQDL | 38 |
| glycoprotein | FLPFFSNVTWFHAIHVSGTNGTKRFDNPVLPFNDGVYFASTEKSNIIRGWIFGT | |
| with six | TLDSKTQSLLIVNNATNVVIKVCEFQFCNDPFLGVYYHKNNKSWMESEFRVYS | |
| proline | SANNCTFEYVSQPFLMDLEGKQGNFKNLREFVFKNIDGYFKIYSKHTPINLVRD | |
| substitutions | LPQGFSALEPLVDLPIGINITRFQTLLALHRSYLTPGDSSSGWTAGAAAYYVGYL | |
| (F817P, | QPRTFLLKYNENGTITDAVDCALDPLSETKCTLKSFTVEKGIYQTSNFRVQPTE | |
| A892P, | SIVRFPNITNLCPFGEVFNATRFASVYAWNRKRISNCVADYSVLYNSASFSTFKC | |
| A899P, | YGVSPTKLNDLCFTNVYADSFVIRGDEVRQIAPGQTGKIADYNYKLPDDFTGCV | |
| A942P, | IAWNSNNLDSKVGGNYNYLYRLFRKSNLKPFERDISTEIYQAGSTPCNGVEGF | |
| K986P, | NCYFPLQSYGFQPTNGVGYQPYRVVVLSFELLHAPATVCGPKKSTNLVKNKCV | |
| V987P) | NFNFNGLTGTGVLTESNKKFLPFQQFGRDIADTTDAVTRAGCLIGAEHVNNSYE | |
| | CDIPIGAGICASYQTQTNRDPQTLEILDITPCSFGGVSVITPGTNTSNQVAVLYQ | |
| | DVNCTEVPVAIHADQLTPTWRVYSTGSNVFQSPRRARSVASQSIIAYTMSLGAE | |
| | NSVAYSNNSIAIPTNFTISVTTEILPVSMTKTSVDCTMYICGDSTECSNLLLQYGS | |
| | FCTQLNRALTGIAVEQDKNTQEVFAQVKQIYKTPPIKDFGGFNFSQILPDPSKPS | |
| | KRSPIEDLLFNKVTLADAGFIKQYGDCLGDIAARDLICAQKFNGLTVLPPLLTDE | |
| | MIAQYTSALLAGTITSGWTFGAGPALQIPFPMQMAYRFNGIGVTQNVLYENQKL | |
| | IANQFNSAIGKIQDSLSSTPSALGKLQDVVNQNAQALNTLVKQLSSNFGAISSVL | |
| | NDILSRLDPPEAEVQIDRLITGRLQSLQTYVTQQLIRAAEIRASANLAATKMSEC | |
| | VLGQSKRVDFCGKGYHLMSFPQSAPHGVVFLHVTYVPAQEKNFTTAPAICHDG | |
| | KAHFPREGVFVSNGTHWFVTQRNFYEPQIITTDNTFVSGNCDVVIGIVNNTVYD | |
| | PLQPELDSFKEELDKYFKNHTSPDVDLGDISGINASVVNIQKEIDRLNEVAKNLN | |
| | ESLIDLQELGKYEQYIKWPWYIWLGFIAGLIAIVMVTIMLCCMTSCCSCLKGCCS | |
| | CGSCCKFDEDDSEPVLKGVKLHYT | |
| Spike | AACCTGACCACCAGGACCCAGCTGCCCCCCGCCTACACCAACAGCTTCAC | 39 |
| protein | CAGGGGCGTGTACTACCCCGACAAGGTGTTCAGGAGCAGCGTGCTGCACA | |
| (Wuhan) | GCACCCAGGACCTGTTCCTGCCCTTCTTCAGCAACGTGACCTGGTTCCAC | |
| with six | GCCATCCACGTGAGCGGCACCAACGGCACCAAGAGGTTCGACAACCCCGT | |
| proline | GCTGCCCTTCAACGACGGCGTGTACTTCGCCAGCACCGAGAAGAGCAACA | |
| substitutions | TCATCAGGGGCTGGATCTTCGGCACCACCCTGGACAGCAAGACCCAGAGC | |
| (F817P, | CTGCTGATCGTGAACAACGCCACCAACGTGGTGATCAAGGTGTGCGAGTTC | |
| A892P, | CAGTTCTGCAACGACCCCTTCCTGGGCGTGTACTACCACAAGAACAACAAG | |
| A899P, | AGCTGGATGGAGAGCGAGTTCAGGGTGTACAGCAGCGCCAACAACTGCAC | |
| A942P, | CTTCGAGTACGTGAGCCAGCCCTTCCTGATGGACCTGGAGGGCAAGCAGG | |
| K986P, | GCAACTTCAAGAACCTGAGGGAGTTCGTGTTCAAGAACATCGACGGCTACT | |
| V987P) | TCAAGATCTACAGCAAGCACACCCCCATCAACCTGGTGAGGGACCTGCCCC | |
| | AGGGCTTCAGCGCCCTGGAGCCCCTGGTGGACCTGCCCATCGGCATCAAC | |
| | ATCACCAGGTTCCAGACCCTGCTGGCCCTGCACAGGAGCTACCTGACCCC | |
| | CGGCGACAGCAGCAGCGGCTGGACCGCCGGCGCCGCCGCCTACTACGTG | |
| | GGCTACCTGCAGCCCAGGACCTTCCTGCTGAAGTACAACGAGAACGGCAC | |
| | CATCACCGACGCCGTGGACTGCGCCCTGGACCCCCTGAGCGAGACCAAGT | |
| | GCACCCTGAAGAGCTTCACCGTGGAGAAGGGCATCTACCAGACCAGCAAC | |
| | TTCAGGGTGCAGCCCACCGAGAGCATCGTGAGGTTCCCCAACATCACCAA | |
| | CCTGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGCGTG | |
| | CTGTACAACAGCGCCAGCTTCAGCACCTTCAAGTGCTACGGCGTGAGCCC | |
| | CACCAAGCTGAACGACCTGTGCTTCACCAACGTGTACGCCGACAGCTTCGT | |
| | GATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATC | |
| | GCCTGGAACAGCAACAACCTGGACAGCAAGGTGGGCGGCAACTACAACTA | |
| | CCTGTACAGGCTGTTCAGGAAGAGCAACCTGAAGCCCTTCGAGAGGGACA | |
| | TCAGCACCGAGATCTACCAGGCCGGCAGCACCCCCTGCAACGGCGTGGAG | |
| | GGCTTCAACTGCTACTTCCCCCTGCAGAGCTACGGCTTCCAGCCCACCAAC | |
| | GGCGTGGGCTACCAGCCCTACAGGGTGGTGGTGCTGAGCTTCGAGCTGCT | |
| | GCACGCCCCCGCCACCGTGTGCGGCCCCAAGAAGAGCACCAACCTGGTG | |
| | AAGAACAAGTGCGTGAACTTCAACTTCAACGGCCTGACCGGCACCGGCGT | |
| | GCTGACCGAGAGCAACAAGAAGTTCCTGCCCTTCCAGCAGTTCGGCAGGG | |
| | ACATCGCCGACACCACCGACGCCGTGAGGGACCCCCAGACCCTGGAGATC | |
| | CTGGACATCACCCCCTGCAGCTTCGGCGGCGTGAGCGTGATCACCCCCGG | |
| | CACCAACACCAGCAACCAGGTGGCCGTGCTGTACCAGGACGTGAACTGCA | |
| | CCGAGGTGCCCGTGGCCATCCACGCCGACCAGCTGACCCCCACCTGGAG | |
| | GGTGTACAGCACCGGCAGCAACGTGTTCCAGACCAGGGCCGGCTGCCTG | |
| | ATCGGCGCCGAGCACGTGAACAACAGCTACGAGTGCGACATCCCCATCGG | |
| | CGCCGGCATCTGCGCCAGCTACCAGACCCAGACCAACAGCCCCGGCAGC | |
| | GCCAGCAGCGTGGCCAGCCAGAGCATCATCGCCTACACCATGAGCCTGGG | |
| | CGCCGAGAACAGCGTGGCCTACAGCAACAACAGCATCGCCATCCCCACCA | |
| | ACTTCACCATCAGCGTGACCACCGAGATCCTGCCCGTGAGCATGACCAAGA | |
| | CCAGCGTGGACTGCACCATGTACATCTGCGGCGACAGCACCGAGTGCAGC | |
| | AACCTGCTGCTGCAGTACGGCAGCTTCTGCACCCAGCTGAACAGGGCCCT | |
| | GACCGGCATCGCCGTGGAGCAGGACAAGAACACCCAGGAGGTGTTCGCC | |
| | CAGGTGAAGCAGATCTACAAGACCCCCCCCATCAAGGACTTCGGCGGCTT | |
| | CAACTTCAGCCAGATCCTGCCCGACCCCAGCAAGCCCAGCAAGAGGAGCC | |
| | CCATCGAGGACCTGCTGTTCAACAAGGTGACCCTGGCCGACGCCGGCTTC | |
| | ATCAAGCAGTACGGCGACTGCCTGGGCGACATCGCCGCCAGGGACCTGAT | |
| | CTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACC | |
| | AGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGT | |
| | ACGAGAACCAGAAGCTGATCGCCAACCAGTTCAACAGCGCCATCGGCAAG | |
| | ATCCAGGACAGCCTGAGCAGCACCCCCAGCGCCCTGGGCAAGCTGCAGG | |
| | ACGTGGTGAACCAGAACGCCCAGGCCCTGAACACCCTGGTGAAGCAGCTG | |
| | AGCAGCAACTTCGGCGCCATCAGCAGCGTGCTGAACGACATCCTGAGCAG | |
| | GCTGGACCCCCCCGAGGCCGAGGTGCAGATCGACAGGCTGATCACCGGC | |
| | AGGCTGCAGAGCCTGCAGACCTACGTGACCCAGCAGCTGATCAGGGCCGC | |
| | CGAGATCAGGGCCAGCGCCAACCTGGCCGCCACCAAGATGAGCGAGTGC | |
| | GTGCTGGGCCAGAGCAAGAGGGTGGACTTCTGCGGCAAGGGCTACCACC | |
| | TGATGAGCTTCCCCCAGAGCGCCCCCCACGGCGTGGTGTTCCTGCACGTG | |
| | ACCTACGTGCCCGCCCAGGAGAAGAACTTCACCACCGCCCCCGCCATCTG | |
| | CCACGACGGCAAGGCCCACTTCCCCAGGGAGGGCGTGTTCGTGAGCAAC | |
| | GGCACCCACTGGTTCGTGACCCAGAGGAACTTCTACGAGCCCCAGATCATC | |
| | ACCACCGACAACACCTTCGTGAGCGGCAACTGCGACGTGGTGATCGGCAT | |
| | CGTGAACAACACCGTGTACGACCCCCTGCAGCCCGAGCTGGACAGCTTCA | |
| | AGGAGGAGCTGGACAAGTACTTCAAGAACCACACCAGCCCCGACGTGGAC | |
| | CTGGGCGACATCAGCGGCATCAACGCCAGCGTGGTGAACATCCAGAAGGA | |
| | GATCGACAGGCTGAACGAGGTGGCCAAGAACCTGAACGAGAGCCTGATCG | |
| | ACCTGCAGGAGCTGGGCAAGTACGAGCAGTACATCAAGTGGCCCTGGTAC | |
| | ATCTGGCTGGGCTTCATCGCCGGCCTGATCGCCATCGTGATGGTGACCATC | |
| | ATGCTGTGCTGCATGACCAGCTGCTGCAGCTGCCTGAAGGGCTGCTGCAG | |
| | CTGCGGCAGCTGCTGCAAGTTCGACGAGGACGACAGCGAGCCCGTGCTG | |
| | AAGGGCGTGAAGCTGCACTACACC | |
| Spike | AACCTGACCACCAGGACCCAGCTGCCCCCCGCCTACACCAACAGCTTCAC | 40 |
| protein | CAGGGGCGTGTACTACCCCGACAAGGTGTTCAGGAGCAGCGTGCTGCACA | |
| (Wuhan) | GCACCCAGGACCTGTTCCTGCCCTTCTTCAGCAACGTGACCTGGTTCCAC | |
| with two | GCCATCCACGTGAGCGGCACCAACGGCACCAAGAGGTTCGACAACCCCGT | |
| proline | GCTGCCCTTCAACGACGGCGTGTACTTCGCCAGCACCGAGAAGAGCAACA | |
| substitutions | TCATCAGGGGCTGGATCTTCGGCACCACCCTGGACAGCAAGACCCAGAGC | |
| (K986P, | CTGCTGATCGTGAACAACGCCACCAACGTGGTGATCAAGGTGTGCGAGTTC | |
| V987P) | CAGTTCTGCAACGACCCCTTCCTGGGCGTGTACTACCACAAGAACAACAAG | |
| | AGCTGGATGGAGAGCGAGTTCAGGGTGTACAGCAGCGCCAACAACTGCAC | |
| | CTTCGAGTACGTGAGCCAGCCCTTCCTGATGGACCTGGAGGGCAAGCAGG | |
| | GCAACTTCAAGAACCTGAGGGAGTTCGTGTTCAAGAACATCGACGGCTACT | |
| | TCAAGATCTACAGCAAGCACACCCCCATCAACCTGGTGAGGGACCTGCCCC | |
| | AGGGCTTCAGCGCCCTGGAGCCCCTGGTGGACCTGCCCATCGGCATCAAC | |
| | ATCACCAGGTTCCAGACCCTGCTGGCCCTGCACAGGAGCTACCTGACCCC | |
| | CGGCGACAGCAGCAGCGGCTGGACCGCCGGCGCCGCCGCCTACTACGTG | |
| | GGCTACCTGCAGCCCAGGACCTTCCTGCTGAAGTACAACGAGAACGGCAC | |
| | CATCACCGACGCCGTGGACTGCGCCCTGGACCCCCTGAGCGAGACCAAGT | |
| | GCACCCTGAAGAGCTTCACCGTGGAGAAGGGCATCTACCAGACCAGCAAC | |
| | TTCAGGGTGCAGCCCACCGAGAGCATCGTGAGGTTCCCCAACATCACCAA | |
| | CCTGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGCGTG | |
| | CTGTACAACAGCGCCAGCTTCAGCACCTTCAAGTGCTACGGCGTGAGCCC | |
| | CACCAAGCTGAACGACCTGTGCTTCACCAACGTGTACGCCGACAGCTTCGT | |
| | GATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATC | |
| | GCCTGGAACAGCAACAACCTGGACAGCAAGGTGGGCGGCAACTACAACTA | |
| | CCTGTACAGGCTGTTCAGGAAGAGCAACCTGAAGCCCTTCGAGAGGGACA | |
| | TCAGCACCGAGATCTACCAGGCCGGCAGCACCCCCTGCAACGGCGTGGAG | |
| | GGCTTCAACTGCTACTTCCCCCTGCAGAGCTACGGCTTCCAGCCCACCAAC | |
| | GGCGTGGGCTACCAGCCCTACAGGGTGGTGGTGCTGAGCTTCGAGCTGCT | |
| | GCACGCCCCCGCCACCGTGTGCGGCCCCAAGAAGAGCACCAACCTGGTG | |
| | AAGAACAAGTGCGTGAACTTCAACTTCAACGGCCTGACCGGCACCGGCGT | |
| | GCTGACCGAGAGCAACAAGAAGTTCCTGCCCTTCCAGCAGTTCGGCAGGG | |
| | ACATCGCCGACACCACCGACGCCGTGAGGGACCCCCAGACCCTGGAGATC | |
| | CTGGACATCACCCCCTGCAGCTTCGGCGGCGTGAGCGTGATCACCCCCGG | |
| | CACCAACACCAGCAACCAGGTGGCCGTGCTGTACCAGGACGTGAACTGCA | |
| | CCGAGGTGCCCGTGGCCATCCACGCCGACCAGCTGACCCCCACCTGGAG | |
| | GGTGTACAGCACCGGCAGCAACGTGTTCCAGACCAGGGCCGGCTGCCTG | |
| | ATCGGCGCCGAGCACGTGAACAACAGCTACGAGTGCGACATCCCCATCGG | |
| | CGCCGGCATCTGCGCCAGCTACCAGACCCAGACCAACAGCCCCAGGAGG | |
| | GCCAGGAGCGTGGCCAGCCAGAGCATCATCGCCTACACCATGAGCCTGGG | |
| | CGCCGAGAACAGCGTGGCCTACAGCAACAACAGCATCGCCATCCCCACCA | |
| | ACTTCACCATCAGCGTGACCACCGAGATCCTGCCCGTGAGCATGACCAAGA | |
| | CCAGCGTGGACTGCACCATGTACATCTGCGGCGACAGCACCGAGTGCAGC | |
| | AACCTGCTGCTGCAGTACGGCAGCTTCTGCACCCAGCTGAACAGGGCCCT | |
| | GACCGGCATCGCCGTGGAGCAGGACAAGAACACCCAGGAGGTGTTCGCC | |
| | CAGGTGAAGCAGATCTACAAGACCCCCCCCATCAAGGACTTCGGCGGCTT | |
| | CAACTTCAGCCAGATCCTGCCCGACCCCAGCAAGCCCAGCAAGAGGAGCT | |
| | TCATCGAGGACCTGCTGTTCAACAAGGTGACCCTGGCCGACGCCGGCTTC | |
| | ATCAAGCAGTACGGCGACTGCCTGGGCGACATCGCCGCCAGGGACCTGAT | |
| | CTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACC | |
| | AGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGT | |
| | ACGAGAACCAGAAGCTGATCGCCAACCAGTTCAACAGCGCCATCGGCAAG | |
| | ATCCAGGACAGCCTGAGCAGCACCGCCAGCGCCCTGGGCAAGCTGCAGG | |
| | ACGTGGTGAACCAGAACGCCCAGGCCCTGAACACCCTGGTGAAGCAGCTG | |
| | AGCAGCAACTTCGGCGCCATCAGCAGCGTGCTGAACGACATCCTGAGCAG | |
| | GCTGGACCCCCCCGAGGCCGAGGTGCAGATCGACAGGCTGATCACCGGC | |
| | AGGCTGCAGAGCCTGCAGACCTACGTGACCCAGCAGCTGATCAGGGCCGC | |
| | CGAGATCAGGGCCAGCGCCAACCTGGCCGCCACCAAGATGAGCGAGTGC | |
| | GTGCTGGGCCAGAGCAAGAGGGTGGACTTCTGCGGCAAGGGCTACCACC | |
| | TGATGAGCTTCCCCCAGAGCGCCCCCCACGGCGTGGTGTTCCTGCACGTG | |
| | ACCTACGTGCCCGCCCAGGAGAAGAACTTCACCACCGCCCCCGCCATCTG | |
| | CCACGACGGCAAGGCCCACTTCCCCAGGGAGGGCGTGTTCGTGAGCAAC | |
| | GGCACCCACTGGTTCGTGACCCAGAGGAACTTCTACGAGCCCCAGATCATC | |
| | ACCACCGACAACACCTTCGTGAGCGGCAACTGCGACGTGGTGATCGGCAT | |
| | CGTGAACAACACCGTGTACGACCCCCTGCAGCCCGAGCTGGACAGCTTCA | |
| | AGGAGGAGCTGGACAAGTACTTCAAGAACCACACCAGCCCCGACGTGGAC | |
| | CTGGGCGACATCAGCGGCATCAACGCCAGCGTGGTGAACATCCAGAAGGA | |
| | GATCGACAGGCTGAACGAGGTGGCCAAGAACCTGAACGAGAGCCTGATCG | |
| | ACCTGCAGGAGCTGGGCAAGTACGAGCAGTACATCAAGTGGCCCTGGTAC | |
| | ATCTGGCTGGGCTTCATCGCCGGCCTGATCGCCATCGTGATGGTGACCATC | |
| | ATGCTGTGCTGCATGACCAGCTGCTGCAGCTGCCTGAAGGGCTGCTGCAG | |
| | CTGCGGCAGCTGCTGCAAGTTCGACGAGGACGACAGCGAGCCCGTGCTG | |
| | AAGGGCGTGAAGCTGCACTACACC | |
| Spike | AACCTGATCACCAGGACCCAGAGCTACACCAACAGCTTCACCAGGGGCGT | 41 |
| protein | GTACTACCCCGACAAGGTGTTCAGGAGCAGCGTGCTGCACAGCACCCAGG | |
| (Omicron | ACCTGTTCCTGCCCTTCTTCAGCAACGTGACCTGGTTCCACGCCATCCACG | |
| BA.2) with 6 | TGAGCGGCACCAACGGCACCAAGAGGTTCGACAACCCCGTGCTGCCCTTC | |
| stabilizing | AACGACGGCGTGTACTTCGCCAGCACCGAGAAGAGCAACATCATCAGGGG | |
| mutations | CTGGATCTTCGGCACCACCCTGGACAGCAAGACCCAGAGCCTGCTGATCG | |
| | TGAACAACGCCACCAACGTGGTGATCAAGGTGTGCGAGTTCCAGTTCTGCA | |
| | ACGACCCCTTCCTGGACGTGTACTACCACAAGAACAACAAGAGCTGGATGG | |
| | AGAGCGAGTTCAGGGTGTACAGCAGCGCCAACAACTGCACCTTCGAGTAC | |
| | GTGAGCCAGCCCTTCCTGATGGACCTGGAGGGCAAGCAGGGCAACTTCAA | |
| | GAACCTGAGGGAGTTCGTGTTCAAGAACATCGACGGCTACTTCAAGATCTA | |
| | CAGCAAGCACACCCCCATCAACCTGGGCAGGGACCTGCCCCAGGGCTTCA | |
| | GCGCCCTGGAGCCCCTGGTGGACCTGCCCATCGGCATCAACATCACCAGG | |
| | TTCCAGACCCTGCTGGCCCTGCACAGGAGCTACCTGACCCCCGGCGACAG | |
| | CAGCAGCGGCTGGACCGCCGGCGCCGCCGCCTACTACGTGGGCTACCTG | |
| | CAGCCCAGGACCTTCCTGCTGAAGTACAACGAGAACGGCACCATCACCGA | |
| | CGCCGTGGACTGCGCCCTGGACCCCCTGAGCGAGACCAAGTGCACCCTG | |
| | AAGAGCTTCACCGTGGAGAAGGGCATCTACCAGACCAGCAACTTCAGGGT | |
| | GCAGCCCACCGAGAGCATCGTGAGGTTCCCCAACATCACCAACCTGTGCC | |
| | CCTTCGACGAGGTGTTCAACGCCACCAGGTTCGCCAGCGTGTACGCCTGG | |
| | AACAGGAAGAGGATCAGCAACTGCGTGGCCGACTACAGCGTGCTGTACAA | |
| | CTTCGCCCCCTTCTTCGCCTTCAAGTGCTACGGCGTGAGCCCCACCAAGCT | |
| | GAACGACCTGTGCTTCACCAACGTGTACGCCGACAGCTTCGTGATCAGGG | |
| | GCAACGAGGTGAGCCAGATCGCCCCCGGCCAGACCGGCAACATCGCCGA | |
| | CTACAACTACAAGCTGCCCGACGACTTCACCGGCTGCGTGATCGCCTGGAA | |
| | CAGCAACAAGCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAACAAGCCCTGCAACGGCGTGGCCGGCTTCAA | |
| | CTGCTACTTCCCCCTGAGGAGCTACGGCTTCAGGCCCACCTACGGCGTGG | |
| | GCCACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGGCGTGAACTGCACCGAGGT | |
| | GCCCGTGGCCATCCACGCCGACCAGCTGACCCCCACCTGGAGGGTGTACA | |
| | GCACCGGCAGCAACGTGTTCCAGACCAGGGCCGGCTGCCTGATCGGCGC | |
| | CGAGTACGTGAACAACAGCTACGAGTGCGACATCCCCATCGGCGCCGGCA | |
| | TCTGCGCCAGCTACCAGACCCAGACCAAGAGCCACCAGCAGGCCCAGAGC | |
| | GTGGCCAGCCAGAGCATCATCGCCTACACCATGAGCCTGGGCGCCGAGAA | |
| | CAGCGTGGCCTACAGCAACAACAGCATCGCCATCCCCACCAACTTCACCAT | |
| | CAGCGTGACCACCGAGATCCTGCCCGTGAGCATGACCAAGACCAGCGTGG | |
| | ACTGCACCATGTACATCTGCGGCGACAGCACCGAGTGCAGCAACCTGCTG | |
| | CTGCAGTACGGCAGCTTCTGCACCCAGCTGAAGAGGGCCCTGACCGGCAT | |
| | CGCCGTGGAGCAGGACAAGAACACCCAGGAGGTGTTCGCCCAGGTGAAG | |
| | CAGATCTACAAGACCCCCCCCATCAAGTACTTCGGCGGCTTCAACTTCAGC | |
| | CAGATCCTGCCCGACCCCAGCAAGCCCAGCAAGAGGAGCCCCATCGAGGA | |
| | CCTGCTGTTCAACAAGGTGACCCTGGCCGACGCCGGCTTCATCAAGCAGT | |
| | ACGGCGACTGCCTGGGCGACATCGCCGCCAGGGACCTGATCTGCGCCCA | |
| | GAAGTTCAACGGCCTGACCGTGCTGCCCCCCCTGCTGACCGACGAGATGA | |
| | TCGCCCAGTACACCAGCGCCCTGCTGGCCGGCACCATCACCAGCGGCTGG | |
| | ACCTTCGGCGCCGGCCCCGCCCTGCAGATCCCCTTCCCCATGCAGATGGC | |
| | CTACAGGTTCAACGGCATCGGCGTGACCCAGAACGTGCTGTACGAGAACC | |
| | AGAAGCTGATCGCCAACCAGTTCAACAGCGCCATCGGCAAGATCCAGGAC | |
| | AGCCTGAGCAGCACCCCCAGCGCCCTGGGCAAGCTGCAGGACGTGGTGA | |
| | ACCACAACGCCCAGGCCCTGAACACCCTGGTGAAGCAGCTGAGCAGCAAG | |
| | TTCGGCGCCATCAGCAGCGTGCTGAACGACATCCTGAGCAGGCTGGACCC | |
| | CCCCGAGGCCGAGGTGCAGATCGACAGGCTGATCACCGGCAGGCTGCAG | |
| | AGCCTGCAGACCTACGTGACCCAGCAGCTGATCAGGGCCGCCGAGATCAG | |
| | GGCCAGCGCCAACCTGGCCGCCACCAAGATGAGCGAGTGCGTGCTGGGC | |
| | CAGAGCAAGAGGGTGGACTTCTGCGGCAAGGGCTACCACCTGATGAGCTT | |
| | CCCCCAGAGCGCCCCCCACGGCGTGGTGTTCCTGCACGTGACCTACGTGC | |
| | CCGCCCAGGAGAAGAACTTCACCACCGCCCCCGCCATCTGCCACGACGGC | |
| | AAGGCCCACTTCCCCAGGGAGGGCGTGTTCGTGAGCAACGGCACCCACT | |
| | GGTTCGTGACCCAGAGGAACTTCTACGAGCCCCAGATCATCACCACCGACA | |
| | ACACCTTCGTGAGCGGCAACTGCGACGTGGTGATCGGCATCGTGAACAAC | |
| | ACCGTGTACGACCCCCTGCAGCCCGAGCTGGACAGCTTCAAGGAGGAGCT | |
| | GGACAAGTACTTCAAGAACCACACCAGCCCCGACGTGGACCTGGGCGACA | |
| | TCAGCGGCATCAACGCCAGCGTGGTGAACATCCAGAAGGAGATCGACAGG | |
| | CTGAACGAGGTGGCCAAGAACCTGAACGAGAGCCTGATCGACCTGCAGGA | |
| | GCTGGGCAAGTACGAGCAGTACATCAAGTGGCCCTGGTACATCTGGCTGG | |
| | GCTTCATCGCCGGCCTGATCGCCATCGTGATGGTGACCATCATGCTGTGCT | |
| | GCATGACCAGCTGCTGCAGCTGCCTGAAGGGCTGCTGCAGCTGCGGCAG | |
| | CTGCTGCAAGTTCGACGAGGACGACAGCGAGCCCGTGCTGAAGGGCGTG | |
| | AAGCTGCACTACACC | |
| Spike | AACCTGATCACCAGGACCCAGAGCTACACCAACAGCTTCACCAGGGGCGT | 42 |
| protein | GTACTACCCCGACAAGGTGTTCAGGAGCAGCGTGCTGCACAGCACCCAGG | |
| (Omicron | ACCTGTTCCTGCCCTTCTTCAGCAACGTGACCTGGTTCCACGCCATCCACG | |
| BA.2) with 2 | TGAGCGGCACCAACGGCACCAAGAGGTTCGACAACCCCGTGCTGCCCTTC | |
| proline | AACGACGGCGTGTACTTCGCCAGCACCGAGAAGAGCAACATCATCAGGGG | |
| substitution | CTGGATCTTCGGCACCACCCTGGACAGCAAGACCCAGAGCCTGCTGATCG | |
| | TGAACAACGCCACCAACGTGGTGATCAAGGTGTGCGAGTTCCAGTTCTGCA | |
| | ACGACCCCTTCCTGGACGTGTACTACCACAAGAACAACAAGAGCTGGATGG | |
| | AGAGCGAGTTCAGGGTGTACAGCAGCGCCAACAACTGCACCTTCGAGTAC | |
| | GTGAGCCAGCCCTTCCTGATGGACCTGGAGGGCAAGCAGGGCAACTTCAA | |
| | GAACCTGAGGGAGTTCGTGTTCAAGAACATCGACGGCTACTTCAAGATCTA | |
| | CAGCAAGCACACCCCCATCAACCTGGGCAGGGACCTGCCCCAGGGCTTCA | |
| | GCGCCCTGGAGCCCCTGGTGGACCTGCCCATCGGCATCAACATCACCAGG | |
| | TTCCAGACCCTGCTGGCCCTGCACAGGAGCTACCTGACCCCCGGCGACAG | |
| | CAGCAGCGGCTGGACCGCCGGCGCCGCCGCCTACTACGTGGGCTACCTG | |
| | CAGCCCAGGACCTTCCTGCTGAAGTACAACGAGAACGGCACCATCACCGA | |
| | CGCCGTGGACTGCGCCCTGGACCCCCTGAGCGAGACCAAGTGCACCCTG | |
| | AAGAGCTTCACCGTGGAGAAGGGCATCTACCAGACCAGCAACTTCAGGGT | |
| | GCAGCCCACCGAGAGCATCGTGAGGTTCCCCAACATCACCAACCTGTGCC | |
| | CCTTCGACGAGGTGTTCAACGCCACCAGGTTCGCCAGCGTGTACGCCTGG | |
| | AACAGGAAGAGGATCAGCAACTGCGTGGCCGACTACAGCGTGCTGTACAA | |
| | CTTCGCCCCCTTCTTCGCCTTCAAGTGCTACGGCGTGAGCCCCACCAAGCT | |
| | GAACGACCTGTGCTTCACCAACGTGTACGCCGACAGCTTCGTGATCAGGG | |
| | GCAACGAGGTGAGCCAGATCGCCCCCGGCCAGACCGGCAACATCGCCGA | |
| | CTACAACTACAAGCTGCCCGACGACTTCACCGGCTGCGTGATCGCCTGGAA | |
| | CAGCAACAAGCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAACAAGCCCTGCAACGGCGTGGCCGGCTTCAA | |
| | CTGCTACTTCCCCCTGAGGAGCTACGGCTTCAGGCCCACCTACGGCGTGG | |
| | GCCACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGGCGTGAACTGCACCGAGGT | |
| | GCCCGTGGCCATCCACGCCGACCAGCTGACCCCCACCTGGAGGGTGTACA | |
| | GCACCGGCAGCAACGTGTTCCAGACCAGGGCCGGCTGCCTGATCGGCGC | |
| | CGAGTACGTGAACAACAGCTACGAGTGCGACATCCCCATCGGCGCCGGCA | |
| | TCTGCGCCAGCTACCAGACCCAGACCAAGAGCCACCAGCAGGCCCAGAGC | |
| | GTGGCCAGCCAGAGCATCATCGCCTACACCATGAGCCTGGGCGCCGAGAA | |
| | CAGCGTGGCCTACAGCAACAACAGCATCGCCATCCCCACCAACTTCACCAT | |
| | CAGCGTGACCACCGAGATCCTGCCCGTGAGCATGACCAAGACCAGCGTGG | |
| | ACTGCACCATGTACATCTGCGGCGACAGCACCGAGTGCAGCAACCTGCTG | |
| | CTGCAGTACGGCAGCTTCTGCACCCAGCTGAAGAGGGCCCTGACCGGCAT | |
| | CGCCGTGGAGCAGGACAAGAACACCCAGGAGGTGTTCGCCCAGGTGAAG | |
| | CAGATCTACAAGACCCCCCCCATCAAGTACTTCGGCGGCTTCAACTTCAGC | |
| | CAGATCCTGCCCGACCCCAGCAAGCCCAGCAAGAGGAGCTTCATCGAGGA | |
| | CCTGCTGTTCAACAAGGTGACCCTGGCCGACGCCGGCTTCATCAAGCAGT | |
| | ACGGCGACTGCCTGGGCGACATCGCCGCCAGGGACCTGATCTGCGCCCA | |
| | GAAGTTCAACGGCCTGACCGTGCTGCCCCCCCTGCTGACCGACGAGATGA | |
| | TCGCCCAGTACACCAGCGCCCTGCTGGCCGGCACCATCACCAGCGGCTGG | |
| | ACCTTCGGCGCCGGCGCCGCCCTGCAGATCCCCTTCGCCATGCAGATGGC | |
| | CTACAGGTTCAACGGCATCGGCGTGACCCAGAACGTGCTGTACGAGAACC | |
| | AGAAGCTGATCGCCAACCAGTTCAACAGCGCCATCGGCAAGATCCAGGAC | |
| | AGCCTGAGCAGCACCGCCAGCGCCCTGGGCAAGCTGCAGGACGTGGTGA | |
| | ACCACAACGCCCAGGCCCTGAACACCCTGGTGAAGCAGCTGAGCAGCAAG | |
| | TTCGGCGCCATCAGCAGCGTGCTGAACGACATCCTGAGCAGGCTGGACCC | |
| | CCCCGAGGCCGAGGTGCAGATCGACAGGCTGATCACCGGCAGGCTGCAG | |
| | AGCCTGCAGACCTACGTGACCCAGCAGCTGATCAGGGCCGCCGAGATCAG | |
| | GGCCAGCGCCAACCTGGCCGCCACCAAGATGAGCGAGTGCGTGCTGGGC | |
| | CAGAGCAAGAGGGTGGACTTCTGCGGCAAGGGCTACCACCTGATGAGCTT | |
| | CCCCCAGAGCGCCCCCCACGGCGTGGTGTTCCTGCACGTGACCTACGTGC | |
| | CCGCCCAGGAGAAGAACTTCACCACCGCCCCCGCCATCTGCCACGACGGC | |
| | AAGGCCCACTTCCCCAGGGAGGGCGTGTTCGTGAGCAACGGCACCCACT | |
| | GGTTCGTGACCCAGAGGAACTTCTACGAGCCCCAGATCATCACCACCGACA | |
| | ACACCTTCGTGAGCGGCAACTGCGACGTGGTGATCGGCATCGTGAACAAC | |
| | ACCGTGTACGACCCCCTGCAGCCCGAGCTGGACAGCTTCAAGGAGGAGCT | |
| | GGACAAGTACTTCAAGAACCACACCAGCCCCGACGTGGACCTGGGCGACA | |
| | TCAGCGGCATCAACGCCAGCGTGGTGAACATCCAGAAGGAGATCGACAGG | |
| | CTGAACGAGGTGGCCAAGAACCTGAACGAGAGCCTGATCGACCTGCAGGA | |
| | GCTGGGCAAGTACGAGCAGTACATCAAGTGGCCCTGGTACATCTGGCTGG | |
| | GCTTCATCGCCGGCCTGATCGCCATCGTGATGGTGACCATCATGCTGTGCT | |
| | GCATGACCAGCTGCTGCAGCTGCCTGAAGGGCTGCTGCAGCTGCGGCAG | |
| | CTGCTGCAAGTTCGACGAGGACGACAGCGAGCCCGTGCTGAAGGGCGTG | |
| | AAGCTGCACTACACC | |
| spike | TACCAAGCTACTAGAGTAGTGGTACTTTCATTTGAGCTTCTAAATGCACCTGC | 43 |
| glycoprotein | CACAGTGTGTGGACCAAAATTGTCCACATCACTAATTAAGAACCAGTGTGTC | |
| | AATTTTAATTTCAATGGACTCAAGGGTACTGGTGTGTTGACTGACTCGTCCA | |
| | AAAAGTTTCAGTCTTTTCAACAATTTGGAAGGGATGCATCTGATTTTACTGAC | |
| | TCAGTACGCGACCCTCAGACACTTCAAATACTTGACATTTCACCATGTTCATT | |
| | TGGTGGTGTGAGTGTAATAACACCAGGAACAAATGCTTCATCTGAAGTAGCC | |
| | GTTCTATACCAAGATGTAAACTGCACTGATGTTCCCACGGCCATACGTGCTG | |
| | ACCAACTCACACCTGCTTGGCGTGTTTACTCTGCTGGAGTAAATGTGTTTCA | |
| | AACTCAGGCTGGCTGTTTAATAGGAGCGGAACATGTCAATGCTTCATATGAG | |
| | TGTGACATTCCCATTGGTGCAGGCATTTGTGCTAGTTACCATACAGCTTCCC | |
| | TTTTACGTAATACAGGCCAGAAATCAATTGTGGCCTATACTATGTCACTTGGT | |
| | GCTGAAAACTCAATTGCTTATGCTAATAACTCAATTGCCATACCTACAAATTTT | |
| | TCAATCAGTGTCACAACTGAAGTGATGCCTGTTTCAATGGCTAAGACATCAG | |
| | TAGATTGTACAATGTACATCTGTGGTGACTCTCAGGAGTGCAGCAACTTACT | |
| | ACTTCAGTATGGTAGCTTTTGCACACAATTAAATCGTGCCCTTTCAGGCATTG | |
| | CTGTTGAACAGGACAAAAACACTCAAGAGGTTTTTGCCCAAGTTAAACAAAT | |
| | GTATAAGACACCAGCCATAAAAGATTTTGGTGGCTTTAATTTCTCACAAATATT | |
| | GCCTGACCCTTCTAAGCCAACAAAAAGATCATTTATTGAGGATTTACTCTTCA | |
| | ACAAAGTGACTCTCGCTGATGCTGGCTTTATGAAGCAATACGGCGAATGCCT | |
| | AGGCGATATTAGTGCTAGAGATCTCATTTGTGCGCAGAAGTTCAATGGACTC | |
| | ACTGTCCTTCCACCTCTACTCACGGATGAAATGATTGCTGCTTACACCGCCG | |
| | CTCTTGTCAGCGGTACTGCTACTGCTGGTTGGACATTTGGTGCAGGTGCTG | |
| | CTCTACAAATACCTTTTGCTATGCAAATGGCTTATAGGTTCAATGGCATTGGA | |
| | GTTACTCAAAATGTTCTCTATGAGAACCAGAAGCAGATCGCTAACCAATTTAA | |
| | CAAGGCGATCAGTCAAATTCAAGAATCACTTACTACTACTTCAACTGCATTG | |
| | GGCAAGCTGCAAGACGTCGTCAACCAGAATGCTCAAGCATTGAACACACTT | |
| | GTTAAACAACTAAGTTCTAACTTTGGTGCAATTTCAAGTGTTTTAAATGACATT | |
| | CTGTCTCGACTYGACAAAGTTGAGGCTGAAGTGCAAATTGATAGGTTGATTA | |
| | CTGGCAGATTACAAAGCCTTCAGACCTATGTAACACAACAACTAATCAGAGC | |
| | TGCTGAAATCAGAGCTTCTGCCAATCTTGCTGCCACTAAGATGTCCGAGTGT | |
| | GTTCTTGGACAATCAAAAAGAGTTGACTTTTGTGGAAAAGGCTATCATCTTAT | |
| | GTCTTTCCCTCAAGCAGCCCCACATGGTGTCGTCTTCTTACATGTCACATAC | |
| | GTGCCATCGCAAGAAAGAAACTTCACCACTGCCCCAGCAATCTGCCATCAA | |
| | GGCAAGGCACACTTCCCTCGTGAAGGTGTTTTTGTATCTAATGGCACTTCTT | |
| | GGTTTATCACACAGAGGAACTTCTTTTCACCACAAATAATTACAACAGACAAT | |
| | ACATTTGTCTCTGGAAATTGTGATGTCGTTATTGGCATCATCAACAATACTGT | |
| | TTATGATCCTCTGCAACCTGAGCTTGACTCATTTAAAGAAGAGCTGGACAAG | |
| | TACTTCAAAAACCACACGTCACCTGATGTRGATCTTGGCGACATCTCAGGCA | |
| | TTAATGCTTCAGTCGTCAATATTCAAAAAGAAATTGACCGCCTCAATGAGGTT | |
| | GCCAAAAATCTAAATGAATCGCTCATCGATCTTCAAGAACTTGGAAAATATGA | |
| | GCA | |

In some embodiments, the large sequences are derived from structural proteins, non-structural proteins, or a combination thereof.

In some embodiments, the one or more large sequences comprises Spike glycoprotein(S) or a portion thereof, Nucleoprotein or a portion thereof, Membrane protein or a portion thereof, and ORF1a/b or a portion thereof. In other embodiments, the one or more large sequences comprises Spike glycoprotein(S) or a portion thereof, Nucleoprotein or a portion thereof, and ORF1a/b or a portion thereof. In further embodiments, the one or more large sequences is selected from the group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein an ORF10 protein. The ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16.

Table 5 shows non-limiting examples of large sequences that may be used in vaccine compositions described herein.

TABLE 5

| | | SEQ |
|---|---|---|
| | | ID |
| | Sequence | NO: |
| NSP1 | ATGGAGAGCCTGGTGCCCGGCTTCAACGAGAAGACCCACGTGCAGCTGAGCCTG | 44 |
| | CCCGTGCTGCAGGTGAGGGACGTGCTGGTGAGGGGCTTCGGCGACAGCGTGGA | |
| | GGAGGTGCTGAGCGAGGCCAGGCAGCACCTGAAGGACGGCACCTGCGGCCTG | |
| | GTGGAGGTGGAGAAGGGCGTGCTGCCCCAGCTGGAGCAGCCCTACGTGTTCATC | |
| | AAGAGGAGCGACGCCAGGACCGCCCCCCACGGCCACGTGATGGTGGAGCTGGT | |
| | GGCCGAGCTGGAGGGCATCCAGTACGGCAGGAGCGGCGAGACCCTGGGCGTGC | |
| | TGGTGCCCCACGTGGGCGAGATCCCCGTGGCCTACAGGAAGGTGCTGCTGAGG | |
| | AAGAACGGCAACAAGGGCGCCGGCGGCCACAGCTACGGCGCCGACCTGAAGAG | |
| | CTTCGACCTGGGCGACGAGCTGGGCACCGACCCCTACGAGGACTTCCAGGAGA | |
| | ACTGGAACACCAAGCACAGCAGCGGCGTGACCAGGGAGCTGATGAGGGAGCTG | |
| | AACGGCGGC | |
| NSP2 | GCCTACACCAGGTACGTGGACAACAACTTCTGCGGCCCCGACGGCTACCCCCTG | 45 |
| | GAGTGCATCAAGGACCTGCTGGCCAGGGCCGGCAAGGCCAGCTGCACCCTGAG | |
| | CGAGCAGCTGGACTTCATCGACACCAAGAGGGGCGTGTACTGCTGCAGGGAGCA | |
| | CGAGCACGAGATCGCCTGGTACACCGAGAGGAGCGAGAAGAGCTACGAGCTGCA | |
| | GACCCCCTTCGAGATCAAGCTGGCCAAGAAGTTCGACACCTTCAACGGCGAGTG | |
| | CCCCAACTTCGTGTTCCCCCTGAACAGCATCATCAAGACCATCCAGCCCAGGGTG | |
| | GAGAAGAAGAAGCTGGACGGCTTCATGGGCAGGATCAGGAGCGTGTACCCCGTG | |
| | GCCAGCCCCAACGAGTGCAACCAGATGTGCCTGAGCACCCTGATGAAGTGCGAC | |
| | CACTGCGGCGAGACCAGCTGGCAGACCGGCGACTTCGTGAAGGCCACCTGCGA | |
| | GTTCTGCGGCACCGAGAACCTGACCAAGGAGGGCGCCACCACCTGCGGCTACC | |
| | TGCCCCAGAACGCCGTGGTGAAGATCTACTGCCCCGCCTGCCACAACAGCGAGG | |
| | TGGGCCCCGAGCACAGCCTGGCCGAGTACCACAACGAGAGCGGCCTGAAGACC | |
| | ATCCTGAGGAAGGGCGGCAGGACCATCGCCTTCGGCGGCTGCGTGTTCAGCTAC | |
| | GTGGGCTGCCACAACAAGTGCGCCTACTGGGTGCCCAGGGCCAGCGCCAACATC | |
| | GGCTGCAACCACACCGGCGTGGTGGGCGAGGGCAGCGAGGGCCTGAACGACAA | |
| | CCTGCTGGAGATCCTGCAGAAGGAGAAGGTGAACATCAACATCGTGGGCGACTT | |
| | CAAGCTGAACGAGGAGATCGCCATCATCCTGGCCAGCTTCAGCGCCAGCACCAG | |
| | CGCCTTCGTGGAGACCGTGAAGGGCCTGGACTACAAGGCCTTCAAGCAGATCGT | |
| | GGAGAGCTGCGGCAACTTCAAGGTGACCAAGGGCAAGGCCAAGAAGGGCGCCT | |
| | GGAACATCGGCGAGCAGAAGAGCATCCTGAGCCCCCTGTACGCCTTCGCCAGCG | |
| | AGGCCGCCAGGGTGGTGAGGAGCATCTTCAGCAGGACCCTGGAGACCGCCCAG | |
| | AACAGCGTGAGGGTGCTGCAGAAGGCCGCCATCACCATCCTGGACGGCATCAGC | |
| | CAGTACAGCCTGAGGCTGATCGACGCCATGATGTTCACCAGCGACCTGGCCACC | |
| | AACAACCTGGTGGTGATGGCCTACATCACCGGCGGCGTGGTGCAGCTGACCAGC | |
| | CAGTGGCTGACCAACATCTTCGGCACCGTGTACGAGAAGCTGAAGCCCGTGCTG | |
| | GACTGGCTGGAGGAGAAGTTCAAGGAGGGCGTGGAGTTCCTGAGGGACGGCTG | |
| | GGAGATCGTGAAGTTCATCAGCACCTGCGCCTGCGAGATCGTGGGCGGCCAGAT | |
| | CGTGACCTGCGCCAAGGAGATCAAGGAGAGCGTGCAGACCTTCTTCAAGCTGGT | |
| | GAACAAGTTCCTGGCCCTGTGCGCCGACAGCATCATCATCGGCGGCGCCAAGCT | |
| | GAAGGCCCTGAACCTGGGCGAGACCTTCGTGACCCACAGCAAGGGCCTGTACAG | |
| | GAAGTGCGTGAAGAGCAGGGAGGAGACCGGCCTGCTGATGCCCCTGAAGGCCC | |
| | CCAAGGAGATCATCTTCCTGGAGGGCGAGACCCTGCCCACCGAGGTGCTGACCG | |
| | AGGAGGTGGTGCTGAAGACCGGCGACCTGCAGCCCCTGGAGCAGCCCACCAGC | |
| | GAGGCCGTGGAGGCCCCCCTGGTGGGCACCCCCGTGTGCATCAACGGCCTGAT | |
| | GCTGCTGGAGATCAAGGACACCGAGAAGTACTGCGCCCTGGCCCCCAACATGAT | |
| | GGTGACCAACAACACCTTCACCCTGAAGGGCGGC | |
| NSP3 | ATGGCCCCCACCAAGGTGACCTTCGGCGACGACACCGTGATCGAGGTGCAGGG | 46 |
| | CTACAAGAGCGTGAACATCACCTTCGAGCTGGACGAGAGGATCGACAAGGTGCT | |
| | GAACGAGAAGTGCAGCGCCTACACCGTGGAGCTGGGCACCGAGGTGAACGAGT | |
| | TCGCCTGCGTGGTGGCCGACGCCGTGATCAAGACCCTGCAGCCCGTGAGCGAG | |
| | CTGCTGACCCCCCTGGGCATCGACCTGGACGAGTGGAGCATGGCCACCTACTAC | |
| | CTGTTCGACGAGAGCGGCGAGTTCAAGCTGGCCAGCCACATGTACTGCAGCTTC | |
| | TACCCCCCCGACGAGGACGAGGAGGAGGGCGACTGCGAGGAGGAGGAGTTCGA | |
| | GCCCAGCACCCAGTACGAGTACGGCACCGAGGACGACTACCAGGGCAAGCCCCT | |
| | GGAGTTCGGCGCCACCAGCGCCGCCCTGCAGCCCGAGGAGGAGCAGGAGGAG | |
| | GACTGGCTGGACGACGACAGCCAGCAGACCGTGGGCCAGCAGGACGGCAGCGA | |
| | GGACAACCAGACCACCACCATCCAGACCATCGTGGAGGTGCAGCCCCAGCTGGA | |
| | GATGGAGCTGACCCCCGTGGTGCAGACCATCGAGGTGAACAGCTTCAGCGGCTA | |
| | CCTGAAGCTGACCGACAACGTGTACATCAAGAACGCCGACATCGTGGAGGAGGC | |
| | CAAGAAGGTGAAGCCCACCGTGGTGGTGAACGCCGCCAACGTGTACCTGAAGCA | |
| | CGGCGGCGGCGTGGCCGGCGCCCTGAACAAGGCCACCAACAACGCCATGCAGG | |
| | TGGAGAGCGACGACTACATCGCCACCAACGGCCCCCTGAAGGTGGGCGGCAGC | |
| | TGCGTGCTGAGCGGCCACAACCTGGCCAAGCACTGCCTGCACGTGGTGGGCCC | |
| | CAACGTGAACAAGGGCGAGGACATCCAGCTGCTGAAGAGCGCCTACGAGAACTT | |
| | CAACCAGCACGAGGTGCTGCTGGCCCCCCTGCTGAGCGCCGGCATCTTCGGCG | |
| | CCGACCCCATCCACAGCCTGAGGGTGTGCGTGGACACCGTGAGGACCAACGTGT | |
| | ACCTGGCCGTGTTCGACAAGAACCTGTACGACAAGCTGGTGAGCAGCTTCCTGG | |
| | AGATGAAGAGCGAGAAGCAGGTGGAGCAGAAGATCGCCGAGATCCCCAAGGAG | |
| | GAGGTGAAGCCCTTCATCACCGAGAGCAAGCCCAGCGTGGAGCAGAGGAAGCA | |
| | GGACGACAAGAAGATCAAGGCCTGCGTGGAGGAGGTGACCACCACCCTGGAGG | |
| | AGACCAAGTTCCTGACCGAGAACCTGCTGCTGTACATCGACATCAACGGCAACCT | |
| | GCACCCCGACAGCGCCACCCTGGTGAGCGACATCGACATCACCTTCCTGAAGAA | |
| | GGACGCCCCCTACATCGTGGGCGACGTGGTGCAGGAGGGCGTGCTGACCGCCG | |
| | TGGTGATCCCCACCAAGAAGGCCGGCGGCACCACCGAGATGCTGGCCAAGGCC | |
| | CTGAGGAAGGTGCCCACCGACAACTACATCACCACCTACCCCGGCCAGGGCCTG | |
| | AACGGCTACACCGTGGAGGAGGCCAAGACCGTGCTGAAGAAGTGCAAGAGCGC | |
| | CTTCTACATCCTGCCCAGCATCATCAGCAACGAGAAGCAGGAGATCCTGGGCACC | |
| | GTGAGCTGGAACCTGAGGGAGATGCTGGCCCACGCCGAGGAGACCAGGAAGCT | |
| | GATGCCCGTGTGCGTGGAGACCAAGGCCATCGTGAGCACCATCCAGAGGAAGTA | |
| | CAAGGGCATCAAGATCCAGGAGGGCGTGGTGGACTACGGCGCCAGGTTCTACTT | |
| | CTACACCAGCAAGACCACCGTGGCCAGCCTGATCAACACCCTGAACGACCTGAA | |
| | CGAGACCCTGGTGACCATGCCCCTGGGCTACGTGACCCACGGCCTGAACCTGGA | |
| | GGAGGCCGCCAGGTACATGAGGAGCCTGAAGGTGCCCGCCACCGTGAGCGTGA | |
| | GCAGCCCCGACGCCGTGACCGCCTACAACGGCTACCTGACCAGCAGCAGCAAGA | |
| | CCCCCGAGGAGCACTTCATCGAGACCATCAGCCTGGCCGGCAGCTACAAGGACT | |
| | GGAGCTACAGCGGCCAGAGCACCCAGCTGGGCATCGAGTTCCTGAAGAGGGGC | |
| | GACAAGAGCGTGTACTACACCAGCAACCCCACCACCTTCCACCTGGACGGCGAG | |
| | GTGATCACCTTCGACAACCTGAAGACCCTGCTGAGCCTGAGGGAGGTGAGGACC | |
| | ATCAAGGTGTTCACCACCGTGGACAACATCAACCTGCAC | |
| NSP5 | AGCGGCTTCAGGAAGATGGCCTTCCCCAGCGGCAAGGTGGAGGGCTGCATGGT | 47 |
| | GCAGGTGACCTGCGGCACCACCACCCTGAACGGCCTGTGGCTGGACGACGTGG | |
| | TGTACTGCCCCAGGCACGTGATCTGCACCAGCGAGGACATGCTGAACCCCAACTA | |
| | CGAGGACCTGCTGATCAGGAAGAGCAACCACAACTTCCTGGTGCAGGCCGGCAA | |
| | CGTGCAGCTGAGGGTGATCGGCCACAGCATGCAGAACTGCGTGCTGAAGCTGAA | |
| | GGTGGACACCGCCAACCCCAAGACCCCCAAGTACAAGTTCGTGAGGATCCAGCC | |
| | CGGCCAGACCTTCAGCGTGCTGGCCTGCTACAACGGCAGCCCCAGCGGCGTGTA | |
| | CCAGTGCGCCATGAGGCCCAACTTCACCATCAAGGGCAGCTTCCTGAACGGCAG | |
| | CTGCGGCAGCGTGGGCTTCAACATCGACTACGACTGCGTGAGCTTCTGCTACATG | |
| | CACCACATGGAGCTGCCCACCGGCGTGCACGCCGGCACCGACCTGGAGGGCAA | |
| | CTTCTACGGCCCCTTCGTGGACAGGCAGACCGCCCAGGCCGCCGGCACCGACA | |
| | CCACCATCACCGTGAACGTGCTGGCCTGGCTGTACGCCGCCGTGATCAACGGCG | |
| | ACAGGTGGTTCCTGAACAGGTTCACCACCACCCTGAACGACTTCAACCTGGTGG | |
| | CCATGAAGTACAACTACGAGCCCCTGACCCAGGACCACGTGGACATCCTGGGCC | |
| | CCCTGAGCGCCCAGACCGGCATCGCCGTGCTGGACATGTGCGCCAGCCTGAAG | |
| | GAGCTGCTGCAGAACGGCATGAACGGCAGGACCATCCTGGGCAGCGCCCTGCT | |
| | GGAGGACGAGTTCACCCCCTTCGACGTGGTGAGGCAGTGCAGCGGCGTGACCT | |
| | TCCAG | |
| NSP6 | AGCGCCGTGAAGAGGACCATCAAGGGCACCCACCACTGGCTGCTGCTGACCATC | 48 |
| | CTGACCAGCCTGCTGGTGCTGGTGCAGAGCACCCAGTGGAGCCTGTTCTTCTTC | |
| | CTGTACGAGAACGCCTTCCTGCCCTTCGCCATGGGCATCATCGCCATGAGCGCCT | |
| | TCGCCATGATGTTCGTGAAGCACAAGCACGCCTTCCTGTGCCTGTTCCTGCTGCC | |
| | CAGCCTGGCCACCGTGGCCTACTTCAACATGGTGTACATGCCCGCCAGCTGGGT | |
| | GATGAGGATCATGACCTGGCTGGACATGGTGGACACCAGCCTGAGCGGCTTCAA | |
| | GCTGAAGGACTGCGTGATGTACGCCAGCGCCGTGGTGCTGCTGATCCTGATGAC | |
| | CGCCAGGACCGTGTACGACGACGGCGCCAGGAGGGTGTGGACCCTGATGAACG | |
| | TGCTGACCCTGGTGTACAAGGTGTACTACGGCAACGCCCTGGACCAGGCCATCA | |
| | GCATGTGGGCCCTGATCATCAGCGTGACCAGCAACTACAGCGGCGTGGTGACCA | |
| | CCGTGATGTTCCTGGCCAGGGGCATCGTGTTCATGTGCGTGGAGTACTGCCCCAT | |
| | CTTCTTCATCACCGGCAACACCCTGCAGTGCATCATGCTGGTGTACTGCTTCCTG | |
| | GGCTACTTCTGCACCTGCTACTTCGGCCTGTTCTGCCTGCTGAACAGGTACTTCA | |
| | GGCTGACCCTGGGCGTGTACGACTACCTGGTGAGCACCCAGGAGTTCAGGTACA | |
| | TGAACAGCCAGGGCCTGCTGCCCCCCAAGAACAGCATCGACGCCTTCAAGCTGA | |
| | ACATCAAGCTGCTGGGCGTGGGCGGCAAGCCCTGCATCAAGGTGGCCACCGTG | |
| | CAG | |
| NSP7 | AGCAAGATGAGCGACGTGAAGTGCACCAGCGTGGTGCTGCTGAGCGTGCTGCA | 49 |
| | GCAGCTGAGGGTGGAGAGCAGCAGCAAGCTGTGGGCCCAGTGCGTGCAGCTGC | |
| | ACAACGACATCCTGCTGGCCAAGGACACCACCGAGGCCTTCGAGAAGATGGTGA | |
| | GCCTGCTGAGCGTGCTGCTGAGCATGCAGGGCGCCGTGGACATCAACAAGCTGT | |
| | GCGAGGAGATGCTGGACAACAGGGCCACCCTGCAG | |
| NSP8 | GCCATCGCCAGCGAGTTCAGCAGCCTGCCCAGCTACGCCGCCTTCGCCACCGCC | 50 |
| | CAGGAGGCCTACGAGCAGGCCGTGGCCAACGGCGACAGCGAGGTGGTGCTGAA | |
| | GAAGCTGAAGAAGAGCCTGAACGTGGCCAAGAGCGAGTTCGACAGGGACGCCG | |
| | CCATGCAGAGGAAGCTGGAGAAGATGGCCGACCAGGCCATGACCCAGATGTACA | |
| | AGCAGGCCAGGAGCGAGGACAAGAGGGCCAAGGTGACCAGCGCCATGCAGACC | |
| | ATGCTGTTCACCATGCTGAGGAAGCTGGACAACGACGCCCTGAACAACATCATCA | |
| | ACAACGCCAGGGACGGCTGCGTGCCCCTGAACATCATCCCCCTGACCACCGCCG | |
| | CCAAGCTGATGGTGGTGATCCCCGACTACAACACCTACAAGAACACCTGCGACGG | |
| | CACCACCTTCACCTACGCCAGCGCCCTGTGGGAGATCCAGCAGGTGGTGGACGC | |
| | CGACAGCAAGATCGTGCAGCTGAGCGAGATCAGCATGGACAACAGCCCCAACCT | |
| | GGCCTGGCCCCTGATCGTGACCGCCCTGAGGGCCAACAGCGCCGTGAAGCTGC | |
| | A | |
| NSP9 | AACAACGAGCTGAGCCCCGTGGCCCTGAGGCAGATGAGCTGCGCCGCCGGCAC | 51 |
| | CACCCAGACCGCCTGCACCGACGACAACGCCCTGGCCTACTACAACACCACCAA | |
| | GGGCGGCAGGTTCGTGCTGGCCCTGCTGAGCGACCTGCAGGACCTGAAGTGGG | |
| | CCAGGTTCCCCAAGAGCGACGGCACCGGCACCATCTACACCGAGCTGGAGCCCC | |
| | CCTGCAGGTTCGTGACCGACACCCCCAAGGGCCCCAAGGTGAAGTACCTGTACT | |
| | TCATCAAGGGCCTGAACAACCTGAACAGGGGCATGGTGCTGGGCAGCCTGGCCG | |
| | CCACCGTGAGGCTGCAG | |
| NSP | GCCGGCAACGCCACCGAGGTGCCCGCCAACAGCACCGTGCTGAGCTTCTGCGC | 52 |
| 10 | CTTCGCCGTGGACGCCGCCAAGGCCTACAAGGACTACCTGGCCAGCGGCGGCC | |
| | AGCCCATCACCAACTGCGTGAAGATGCTGTGCACCCACACCGGCACCGGCCAGG | |
| | CCATCACCGTGACCCCCGAGGCCAACATGGACCAGGAGAGCTTCGGCGGCGCC | |
| | AGCTGCTGCCTGTACTGCAGGTGCCACATCGACCACCCCAACCCCAAGGGCTTC | |
| | TGCGACCTGAAGGGCAAGTACGTGCAGATCCCCACCACCTGCGCCAACGACCCC | |
| | GTGGGCTTCACCCTGAAGAACACCGTGTGCACCGTGTGCGGCATGTGGAAGGGC | |
| | TACGGCTGCAGCTGCGACCAGCTGAGGGAGCCCATGCTGCAG | |
| NSP | ATGAGCGCCGACGCCCAGAGCTTCCTGAACAGGGTGTGCGGCGTGAGCGCCGC | 53 |
| 12 | CAGGCTGACCCCCTGCGGCACCGGCACCAGCACCGACGTGGTGTACAGGGCCT | |
| | TCGACATCTACAACGACAAGGTGGCCGGCTTCGCCAAGTTCCTGAAGACCAACTG | |
| | CTGCAGGTTCCAGGAGAAGGACGAGGACGACAACCTGATCGACAGCTACTTCGT | |
| | GGTGAAGAGGCACACCTTCAGCAACTACCAGCACGAGGAGACCATCTACAACCT | |
| | GCTGAAGGACTGCCCCGCCGTGGCCAAGCACGACTTCTTCAAGTTCAGGATCGA | |
| | CGGCGACATGGTGCCCCACATCAGCAGGCAGAGGCTGACCAAGTACACCATGGC | |
| | CGACCTGGTGTACGCCCTGAGGCACTTCGACGAGGGCAACTGCGACACCCTGAA | |
| | GGAGATCCTGGTGACCTACAACTGCTGCGACGACGACTACTTCAACAAGAAGGAC | |
| | TGGTACGACTTCGTGGAGAACCCCGACATCCTGAGGGTGTACGCCAACCTGGGC | |
| | GAGAGGGTGAGGCAGGCCCTGCTGAAGACCGTGCAGTTCTGCGACGCCATGAG | |
| | GAACGCCGGCATCGTGGGCGTGCTGACCCTGGACAACCAGGACCTGAACGGCA | |
| | ACTGGTACGACTTCGGCGACTTCATCCAGACCACCCCCGGCAGCGGCGTGCCCG | |
| | TGGTGGACAGCTACTACAGCCTGCTGATGCCCATCCTGACCCTGACCAGGGCCC | |
| | TGACCGCCGAGAGCCACGTGGACACCGACCTGACCAAGCCCTACATCAAGTGGG | |
| | ACCTGCTGAAGTACGACTTCACCGAGGAGAGGCTGAAGCTGTTCGACAGGTACTT | |
| | CAAGTACTGGGACCAGACCTACCACCCCAACTGCGTGAACTGCCTGGACGACAG | |
| | GTGCATCCTGCACTGCGCCAACTTCAACGTGCTGTTCAGCACCGTGTTCCCCCCC | |
| | ACCAGCTTCGGCCCCCTGGTGAGGAAGATCTTCGTGGACGGCGTGCCCTTCGTG | |
| | GTGAGCACCGGCTACCACTTCAGGGAGCTGGGCGTGGTGCACAACCAGGACGT | |
| | GAACCTGCACAGCAGCAGGCTGAGCTTCAAGGAGCTGCTGGTGTACGCCGCCGA | |
| | CCCCGCCATGCACGCCGCCAGCGGCAACCTGCTGCTGGACAAGAGGACCACCT | |
| | GCTTCAGCGTGGCCGCCCTGACCAACAACGTGGCCTTCCAGACCGTGAAGCCCG | |
| | GCAACTTCAACAAGGACTTCTACGACTTCGCCGTGAGCAAGGGCTTCTTCAAGGA | |
| | GGGCAGCAGCGTGGAGCTGAAGCACTTCTTCTTCGCCCAGGACGGCAACGCCG | |
| | CCATCAGCGACTACGACTACTACAGGTACAACCTGCCCACCATGTGCGACATCAG | |
| | GCAGCTGCTGTTCGTGGTGGAGGTGGTGGACAAGTACTTCGACTGCTACGACGG | |
| | CGGCTGCATCAACGCCAACCAGGTGATCGTGAACAACCTGGACAAGAGCGCCGG | |
| | CTTCCCCTTCAACAAGTGGGGCAAGGCCAGGCTGTACTACGACAGCATGAGCTAC | |
| | GAGGACCAGGACGCCCTGTTCGCCTACACCAAGAGGAACGTGATCCCCACCATC | |
| | ACCCAGATGAACCTGAAGTACGCCATCAGCGCCAAGAACAGGGCCAGGACCGTG | |
| | GCCGGCGTGAGCATCTGCAGCACCATGACCAACAGGCAGTTCCACCAGAAGCTG | |
| | CTGAAGAGCATCGCCGCCACCAGGGGCGCCACCGTGGTGATCGGCACCAGCAA | |
| | GTTCTACGGCGGCTGGCACAACATGCTGAAGACCGTGTACAGCGACGTGGAGAA | |
| | CCCCCACCTGATGGGCTGGGACTACCCCAAGTGCGACAGGGCCATGCCCAACAT | |
| | GCTGAGGATCATGGCCAGCCTGGTGCTGGCCAGGAAGCACACCACCTGCTGCAG | |
| | CCTGAGCCACAGGTTCTACAGGCTGGCCAACGAGTGCGCCCAGGTGCTGAGCG | |
| | AGATGGTGATGTGCGGCGGCAGCCTGTACGTGAAGCCCGGCGGCACCAGCAGC | |
| | GGCGACGCCACCACCGCCTACGCCAACAGCGTGTTCAACATCTGCCAGGCCGTG | |
| | ACCGCCAACGTGAACGCCCTGCTGAGCACCGACGGCAACAAGATCGCCGACAAG | |
| | TACGTGAGGAACCTGCAGCACAGGCTGTACGAGTGCCTGTACAGGAACAGGGAC | |
| | GTGGACACCGACTTCGTGAACGAGTTCTACGCCTACCTGAGGAAGCACTTCAGCA | |
| | TGATGATCCTGAGCGACGACGCCGTGGTGTGCTTCAACAGCACCTACGCCAGCC | |
| | AGGGCCTGGTGGCCAGCATCAAGAACTTCAAGAGCGTGCTGTACTACCAGAACAA | |
| | CGTGTTCATGAGCGAGGCCAAGTGCTGGACCGAGACCGACCTGACCAAGGGCC | |
| | CCCACGAGTTCTGCAGCCAGCACACCATGCTGGTGAAGCAGGGCGACGACTACG | |
| | TGTACCTGCCCTACCCCGACCCCAGCAGGATCCTGGGCGCCGGCTGCTTCGTGG | |
| | ACGACATCGTGAAGACCGACGGCACCCTGATGATCGAGAGGTTCGTGAGCCTGG | |
| | CCATCGACGCCTACCCCCTGACCAAGCACCCCAACCAGGAGTACGCCGACGTGT | |
| | TCCACCTGTACCTGCAGTACATCAGGAAGCTGCACGACGAGCTGACCGGCCACAT | |
| | GCTGGACATGTACAGCGTGATGCTGACCAACGACAACACCAGCAGGTACTGGGA | |
| | GCCCGAGTTCTACGAGGCCATGTACACCCCCCACACCGTGCTGCAG | |
| NSP | GCCGTGGGCGCCTGCGTGCTGTGCAACAGCCAGACCAGCCTGAGGTGCGGCGC | 54 |
| 13 | CTGCATCAGGAGGCCCTTCCTGTGCTGCAAGTGCTGCTACGACCACGTGATCAG | |
| | CACCAGCCACAAGCTGGTGCTGAGCGTGAACCCCTACGTGTGCAACGCCCCCGG | |
| | CTGCGACGTGACCGACGTGACCCAGCTGTACCTGGGCGGCATGAGCTACTACTG | |
| | CAAGAGCCACAAGCCCCCCATCAGCTTCCCCCTGTGCGCCAACGGCCAGGTGTT | |
| | CGGCCTGTACAAGAACACCTGCGTGGGCAGCGACAACGTGACCGACTTCAACGC | |
| | CATCGCCACCTGCGACTGGACCAACGCCGGCGACTACATCCTGGCCAACACCTG | |
| | CACCGAGAGGCTGAAGCTGTTCGCCGCCGAGACCCTGAAGGCCACCGAGGAGA | |
| | CCTTCAAGCTGAGCTACGGCATCGCCACCGTGAGGGAGGTGCTGAGCGACAGG | |
| | GAGCTGCACCTGAGCTGGGAGGTGGGCAAGCCCAGGCCCCCCCTGAACAGGAA | |
| | CTACGTGTTCACCGGCTACAGGGTGACCAAGAACAGCAAGGTGCAGATCGGCGA | |
| | GTACACCTTCGAGAAGGGCGACTACGGCGACGCCGTGGTGTACAGGGGCACCAC | |
| | CACCTACAAGCTGAACGTGGGCGACTACTTCGTGCTGACCAGCCACACCGTGAT | |
| | GCCCCTGAGCGCCCCCACCCTGGTGCCCCAGGAGCACTACGTGAGGATCACCG | |
| | GCCTGTACCCCACCCTGAACATCAGCGACGAGTTCAGCAGCAACGTGGCCAACT | |
| | ACCAGAAGGTGGGCATGCAGAAGTACAGCACCCTGCAGGGCCCCCCCGGCACC | |
| | GGCAAGAGCCACTTCGCCATCGGCCTGGCCCTGTACTACCCCAGCGCCAGGATC | |
| | GTGTACACCGCCTGCAGCCACGCCGCCGTGGACGCCCTGTGCGAGAAGGCCCT | |
| | GAAGTACCTGCCCATCGACAAGTGCAGCAGGATCATCCCCGCCAGGGCCAGGGT | |
| | GGAGTGCTTCGACAAGTTCAAGGTGAACAGCACCCTGGAGCAGTACGTGTTCTG | |
| | CACCGTGAACGCCCTGCCCGAGACCACCGCCGACATCGTGGTGTTCGACGAGAT | |
| | CAGCATGGCCACCAACTACGACCTGAGCGTGGTGAACGCCAGGCTGAGGGCCAA | |
| | GCACTACGTGTACATCGGCGACCCCGCCCAGCTGCCCGCCCCCAGGACCCTGCT | |
| | GACCAAGGGCACCCTGGAGCCCGAGTACTTCAACAGCGTGTGCAGGCTGATGAA | |
| | GACCATCGGCCCCGACATGTTCCTGGGCACCTGCAGGAGGTGCCCCGCCGAGAT | |
| | CGTGGACACCGTGAGCGCCCTGGTGTACGACAACAAGCTGAAGGCCCACAAGGA | |
| | CAAGAGCGCCCAGTGCTTCAAGATGTTCTACAAGGGCGTGATCACCCACGACGT | |
| | GAGCAGCGCCATCAACAGGCCCCAGATCGGCGTGGTGAGGGAGTTCCTGACCA | |
| | GGAACCCCGCCTGGAGGAAGGCCGTGTTCATCAGCCCCTACAACAGCCAGAACG | |
| | CCGTGGCCAGCAAGATCCTGGGCCTGCCCACCCAGACCGTGGACAGCAGCCAG | |
| | GGCAGCGAGTACGACTACGTGATCTTCACCCAGACCACCGAGACCGCCCACAGC | |
| | TGCAACGTGAACAGGTTCAACGTGGCCATCACCAGGGCCAAGGTGGGCATCCTG | |
| | TGCATCATGAGCGACAGGGACCTGTACGACAAGCTGCAGTTCACCAGCCTGGAG | |
| | ATCCCCAGGAGGAACGTGGCCACCCTGCAG | |
| NSP | GCCGAGAACGTGACCGGCCTGTTCAAGGACTGCAGCAAGGTGATCACCGGCCTG | 55 |
| 14 | CACCCCACCCAGGCCCCCACCCACCTGAGCGTGGACACCAAGTTCAAGACCGAG | |
| | GGCCTGTGCGTGGACATCCCCGGCATCCCCAAGGACATGACCTACAGGAGGCTG | |
| | ATCAGCATGATGGGCTTCAAGATGAACTACCAGGTGAACGGCTACCCCAACATGTT | |
| | CATCACCAGGGAGGAGGCCATCAGGCACGTGAGGGCCTGGATCGGCTTCGACGT | |
| | GGAGGGCTGCCACGCCACCAGGGAGGCCGTGGGCACCAACCTGCCCCTGCAGC | |
| | TGGGCTTCAGCACCGGCGTGAACCTGGTGGCCGTGCCCACCGGCTACGTGGAC | |
| | ACCCCCAACAACACCGACTTCAGCAGGGTGAGCGCCAAGCCCCCCCCCGGCGA | |
| | CCAGTTCAAGCACCTGATCCCCCTGATGTACAAGGGCCTGCCCTGGAACGTGGT | |
| | GAGGATCAAGATCGTGCAGATGCTGAGCGACACCCTGAAGAACCTGAGCGACAG | |
| | GGTGGTGTTCGTGCTGTGGGCCCACGGCTTCGAGCTGACCAGCATGAAGTACTT | |
| | CGTGAAGATCGGCCCCGAGAGGACCTGCTGCCTGTGCGACAGGAGGGCCACCT | |
| | GCTTCAGCACCGCCAGCGACACCTACGCCTGCTGGCACCACAGCATCGGCTTCG | |
| | ACTACGTGTACAACCCCTTCATGATCGACGTGCAGCAGTGGGGCTTCACCGGCAA | |
| | CCTGCAGAGCAACCACGACCTGTACTGCCAGGTGCACGGCAACGCCCACGTGG | |
| | CCAGCTGCGACGCCATCATGACCAGGTGCCTGGCCGTGCACGAGTGCTTCGTGA | |
| | AGAGGGTGGACTGGACCATCGAGTACCCCATCATCGGCGACGAGCTGAAGATCA | |
| | ACGCCGCCTGCAGGAAGGTGCAGCACATGGTGGTGAAGGCCGCCCTGCTGGCC | |
| | GACAAGTTCCCCGTGCTGCACGACATCGGCAACCCCAAGGCCATCAAGTGCGTG | |
| | CCCCAGGCCGACGTGGAGTGGAAGTTCTACGACGCCCAGCCCTGCAGCGACAA | |
| | GGCCTACAAGATCGAGGAGCTGTTCTACAGCTACGCCACCCACAGCGACAAGTTC | |
| | ACCGACGGCGTGTGCCTGTTCTGGAACTGCAACGTGGACAGGTACCCCGCCAAC | |
| | AGCATCGTGTGCAGGTTCGACACCAGGGTGCTGAGCAACCTGAACCTGCCCGGC | |
| | TGCGACGGCGGCAGCCTGTACGTGAACAAGCACGCCTTCCACACCCCCGCCTTC | |
| | GACAAGAGCGCCTTCGTGAACCTGAAGCAGCTGCCCTTCTTCTACTACAGCGACA | |
| | GCCCCTGCGAGAGCCACGGCAAGCAGGTGGTGAGCGACATCGACTACGTGCCC | |
| | CTGAAGAGCGCCACCTGCATCACCAGGTGCAACCTGGGCGGCGCCGTGTGCAG | |
| | GCACCACGCCAACGAGTACAGGCTGTACCTGGACGCCTACAACATGATGATCAGC | |
| | GCCGGCTTCAGCCTGTGGGTGTACAAGCAGTTCGACACCTACAACCTGTGGAAC | |
| | ACCTTCACCAGGCTGCAG | |
| NSP | AGCCTGGAGAACGTGGCCTTCAACGTGGTGAACAAGGGCCACTTCGACGGCCAG | 56 |
| 15 | CAGGGCGAGGTGCCCGTGAGCATCATCAACAACACCGTGTACACCAAGGTGGAC | |
| | GGCGTGGACGTGGAGCTGTTCGAGAACAAGACCACCCTGCCCGTGAACGTGGC | |
| | CTTCGAGCTGTGGGCCAAGAGGAACATCAAGCCCGTGCCCGAGGTGAAGATCCT | |
| | GAACAACCTGGGCGTGGACATCGCCGCCAACACCGTGATCTGGGACTACAAGAG | |
| | GGACGCCCCCGCCCACATCAGCACCATCGGCGTGTGCAGCATGACCGACATCGC | |
| | CAAGAAGCCCACCGAGACCATCTGCGCCCCCCTGACCGTGTTCTTCGACGGCAG | |
| | GGTGGACGGCCAGGTGGACCTGTTCAGGAACGCCAGGAACGGCGTGCTGATCA | |
| | CCGAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTACTA | |
| | CAAGAAGGTGGACGGCGTGGTGCAGCAGCTGCCCGAGACCTACTTCACCCAGA | |
| | GCAGGAACCTGCAGGAGTTCAAGCCCAGGAGCCAGATGGAGATCGACTTCCTGG | |
| | AGCTGGCCATGGACGAGTTCATCGAGAGGTACAAGCTGGAGGGCTACGCCTTCG | |
| | AGCACATCGTGTACGGCGACTTCAGCCACAGCCAGCTGGGCGGCCTGCACCTGC | |
| | TGATCGGCCTGGCCAAGAGGTTCAAGGAGAGCCCCTTCGAGCTGGAGGACTTCA | |
| | TCCCCATGGACAGCACCGTGAAGAACTACTTCATCACCGACGCCCAGACCGGCA | |
| | GCAGCAAGTGCGTGTGCAGCGTGATCGACCTGCTGCTGGACGACTTCGTGGAGA | |
| | TCATCAAGAGCCAGGACCTGAGCGTGGTGAGCAAGGTGGTGAAGGTGACCATCG | |
| | ACTACACCGAGATCAGCTTCATGCTGTGGTGCAAGGACGGCCACGTGGAGACCT | |
| | TCTACCCCAAGCTGCAG | |
| NSP | AGCAGCCAGGCCTGGCAGCCCGGCGTGGCCATGCCCAACCTGTACAAGATGCAG | 57 |
| 16 | AGGATGCTGCTGGAGAAGTGCGACCTGCAGAACTACGGCGACAGCGCCACCCTG | |
| | CCCAAGGGCATCATGATGAACGTGGCCAAGTACACCCAGCTGTGCCAGTACCTGA | |
| | ACACCCTGACCCTGGCCGTGCCCTACAACATGAGGGTGATCCACTTCGGCGCCG | |
| | GCAGCGACAAGGGCGTGGCCCCCGGCACCGCCGTGCTGAGGCAGTGGCTGCC | |
| | CACCGGCACCCTGCTGGTGGACAGCGACCTGAACGACTTCGTGAGCGACGCCG | |
| | ACAGCACCCTGATCGGCGACTGCGCCACCGTGCACACCGCCAACAAGTGGGAC | |
| | CTGATCATCAGCGACATGTACGACCCCAAGACCAAGAACGTGACCAAGGAGAACG | |
| | ACAGCAAGGAGGGCTTCTTCACCTACATCTGCGGCTTCATCCAGCAGAAGCTGGC | |
| | CCTGGGCGGCAGCGTGGCCATCAAGATCACCGAGCACAGCTGGAACGCCGACC | |
| | TGTACAAGCTGATGGGCCACTTCGCCTGGTGGACCGCCTTCGTGACCAACGTGA | |
| | ACGCCAGCAGCAGCGAGGCCTTCCTGATCGGCTGCAACTACCTGGGCAAGCCCA | |
| | GGGAGCAGATCGACGGCTACGTGATGCACGCCAACTACATCTTCTGGAGGAACAC | |
| | CAACCCCATCCAGCTGAGCAGCTACAGCCTGTTCGACATGAGCAAGTTCCCCCTG | |
| | AAGCTGAGGGGCACCGCCGTGATGAGCCTGAAGGAGGGCCAGATCAACGACATG | |
| | ATCCTGAGCCTGCTGAGCAAGGGCAGGCTGATCATCAGGGAGAACAACAGGGTG | |
| | GTGATCAGCAGCGACGTGCTGGTGAACAAC | |
| Orf3a | ATGGACCTGTTCATGAGGATCTTCACCATCGGCACCGTGACCCTGAAGCAGGGC | 58 |
| | GAGATCAAGGACGCCACCCCCAGCGACTTCGTGAGGGCCACCGCCACCATCCCC | |
| | ATCCAGGCCAGCCTGCCCTTCGGCTGGCTGATCGTGGGCGTGGCCCTGCTGGC | |
| | CGTGTTCCAGAGCGCCAGCAAGATCATCACCCTGAAGAAGAGGTGGCAGCTGGC | |
| | CCTGAGCAAGGGCGTGCACTTCGTGTGCAACCTGCTGCTGCTGTTCGTGACCGT | |
| | GTACAGCCACCTGCTGCTGGTGGCCGCCGGCCTGGAGGCCCCCTTCCTGTACCT | |
| | GTACGCCCTGGTGTACTTCCTGCAGAGCATCAACTTCGTGAGGATCATCATGAGG | |
| | CTGTGGCTGTGCTGGAAGTGCAGGAGCAAGAACCCCCTGCTGTACGACGCCAAC | |
| | TACTTCCTGTGCTGGCACACCAACTGCTACGACTACTGCATCCCCTACAACAGCGT | |
| | GACCAGCAGCATCGTGATCACCAGCGGCGACGGCACCACCAGCCCCATCAGCGA | |
| | GCACGACTACCAGATCGGCGGCTACACCGAGAAGTGGGAGAGCGGCGTGAAGG | |
| | ACTGCGTGGTGCTGCACAGCTACTTCACCAGCGACTACTACCAGCTGTACAGCAC | |
| | CCAGCTGAGCACCGACACCGGCGTGGAGCACGTGACCTTCTTCATCTACAACAA | |
| | GATCGTGGACGAGCCCGAGGAGCACGTGCAGATCCACACCATCGACGGCAGCA | |
| | GCGGCGTGGTGAACCCCGTGATGGAGCCCATCTACGACGAGCCCACCACCACCA | |
| | CCAGCGTGCCCCTG | |
| Nucleo- | AGCGACAACGGCCCCCAGAACCAGAGGAACGCCCCCAGGATCACCTTCGGCGG | 59 |
| capsid | CCCCAGCGACAGCACCGGCAGCAACCAGAACGGCGAGAGGAGCGGCGCCAGG | |
| | AGCAAGCAGAGGAGGCCCCAGGGCCTGCCCAACAACACCGCCAGCTGGTTCAC | |
| | CGCCCTGACCCAGCACGGCAAGGAGGACCTGAAGTTCCCCAGGGGCCAGGGCG | |
| | TGCCCATCAACACCAACAGCAGCCCCGACGACCAGATCGGCTACTACAGGAGGG | |
| | CCACCAGGAGGATCAGGGGCGGCGACGGCAAGATGAAGGACCTGAGCCCCAGG | |
| | TGGTACTTCTACTACCTGGGCACCGGCCCCGAGGCCGGCCTGCCCTACGGCGCC | |
| | AACAAGGACGGCATCATCTGGGTGGCCACCGAGGGCGCCCTGAACACCCCCAAG | |
| | GACCACATCGGCACCAGGAACCCCGCCAACAACGCCGCCATCGTGCTGCAGCTG | |
| | CCCCAGGGCACCACCCTGCCCAAGGGCTTCTACGCCGAGGGCAGCAGGGGCGG | |
| | CAGCCAGGCCAGCAGCAGGAGCAGCAGCAGGAGCAGGAACAGCAGCAGGAACA | |
| | GCACCCCCGGCAGCAGCAGGGGCACCAGCCCCGCCAGGATGGCCGGCAACGG | |
| | CGGCGACGCCGCCCTGGCCCTGCTGCTGCTGGACAGGCTGAACCAGCTGGAGA | |
| | GCAAGATGAGCGGCAAGGGCCAGCAGCAGCAGGGCCAGACCGTGACCAAGAAG | |
| | AGCGCCGCCGAGGCCAGCAAGAAGCCCAGGCAGAAGAGGACCGCCACCAAGGC | |
| | CTACAACGTGACCCAGGCCTTCGGCAGGAGGGGCCCCGAGCAGACCCAGGGCA | |
| | ACTTCGGCGACCAGGAGCTGATCAGGCAGGGCACCGACTACAAGCACTGGCCCC | |
| | AGATCGCCCAGTTCGCCCCCAGCGCCAGCGCCTTCTTCGGCATGAGCAGGATCG | |
| | GCATGGAGGTGACCCCCAGCGGCACCTGGCTGACCTACACCGGCGCCATCAAGC | |
| | TGGACGACAAGGACCCCAACTTCAAGGACCAGGTGATCCTGCTGAACAAGCACAT | |
| | CGACGCCTACAAGACCTTCCCCCCCACCGAGCCCAAGAAGGACAAGAAGAAGAA | |
| | GGCCGACGAGACCCAGGCCCTGCCCCAGAGGCAGAAGAAGCAGCAGACCGTGA | |
| | CCCTGCTGCCCGCCGCCGACCTGGACGACTTCAGCAAGCAGCTGCAGCAGAGC | |
| | ATGAGCAGCGCCGACAGCACCCAGGCC | |
| Orf1a/b | GACCAGGAACCCCGCCTGGAGGAAGGCCGTGTTCATCAGCCCCTACAACAGCCA | 60 |
| | GAACGCCGTGGCCAGCAAGATCCTGGGCCTGCCCACCCAGACCGTGGACAGCA | |
| | GCCAGGGCAGCGAGTACGACTACGTGATCTTCACCCAGACCACCGAGACCGCCC | |
| | ACAGCTGCAACGTGAACAGGTTCAACGTGGCCATCACCAGGGCCAAGGTGGGCA | |
| | TCCTGTGCATCATGAGCGACAGGGACCTGTACGACAAGCTGCAGTTCACCAGCCT | |
| | GGAGATCCCCAGGAGGAACGTGGCCACCCTGCAGGCCGAGAACGTGACCGGCC | |
| | TGTTCAAGGACTGCAGCAAGGTGATCACCGGCCTGCACCCCACCCAGGCCCCCA | |
| | CCCACCTGAGCGTGGACACCAAGTTCAAGACCGAGGGCCTGTGCGTGGACATCC | |
| | CCGGCATCCCCAAGGACATGACCTACAGGAGGCTGATCAGCATGATGGGCTTCAA | |
| | GATGAACTACCAGGTGAACGGCTACCCCAACATGTTCATCACCAGGGAGGAGGCC | |
| | ATCAGGCACGTGAGGGCCTGGATCGGCTTCGACGTGGAGGGCTGCCACGCCAC | |
| | CAGGGAGGCCGTGGGCACCAACCTGCCCCTGCAGCTGGGCTTCAGCACCGGCG | |
| | TGAACCTGGTGGCCGTGCCCACCGGCTACGTGGACACCCCCAACAACACCGACT | |
| | TCAGCAGGGTGAGCGCCAAGCCCCCCCCCGGCGACCAGTTCAAGCACCTGATC | |
| | CCCCTGATGTACAAGGGCCTGCCCTGGAACGTGGTGAGGATCAAGATCGTGCAG | |
| | ATGCTGAGCGACACCCTGAAGAACCTGAGCGACAGGGTGGTGTTCGTGCTGTGG | |
| | GCCCACGGCTTCGAGCTGACCAGCATGAAGTACTTCGTGAAGATCGGCCCCGAG | |
| | AGGACCTGCTGCCTGTGCGACAGGAGGGCCACCTGCTTCAGCACCGCCAGCGA | |
| | CACCTACGCCTGCTGGCACCACAGCATCGGCTTCGACTACGTGTACAACCCCTTC | |
| | ATGATCGACGTGCAGCAGTGGGGCTTCACCGGCAACCTGCAGAGCAACCACGAC | |
| | CTGTACTGCCAGGTGCACGGCAACGCCCACGTGGCCAGCTGCGACGCCATCATG | |
| | ACCAGGTGCCTGGCCGTGCACGAGTGCTTCGTGAAGAGGGTGGACTGGACCATC | |
| | GAGTACCCCATCATCGGCGACGAGCTGAAGATCAACGCCGCCTGCAGGAAGGTG | |
| | CAGCACATGGTGGTGAAGGCCGCCCTGCTGGCCGACAAGTTCCCCGTGCTGCAC | |
| | GACATCGGCAACCCCAAGGCCATCAAGTGCGTGCCCCAGGCCGACGTGGAGTG | |
| | GAAGTTCTACGACGCCCAGCCCTGCAGCGACAAGGCCTACAAGATCGAGGAGCT | |
| | GTTCTACAGCTACGCCACCCACAGCGACAAGTTCACCGACGGCGTGTGCCTGTT | |
| | CTGGAACTGCAACGTGGACAGGTACCCCGCCAACAGCATCGTGTGCAGGTTCGA | |
| | CACCAGGGTGCTGAGCAACCTGAACCTGCCCGGCTGCGACGGCGGCAGCCTGT | |
| | ACGTGAACAAGCACGCCTTCCACACCCCCGCCTTCGACAAGAGCGCCTTCGTGA | |
| | ACCTGAAGCAGCTGCCCTTCTTCTACTACAGCGACAGCCCCTGCGAGAGCCACG | |
| | GCAAGCAGGTGGTGAGCGACATCGACTACGTGCCCCTGAAGAGCGCCACCTGCA | |
| | TCACCAGGTGCAACCTGGGCGGCGCCGTGTGCAGGCACCACGCCAACGAGTAC | |
| | AGGCTGTACCTGGACGCCTACAACATGATGATCAGCGCCGGCTTCAGCCTGTGG | |
| | GTGTACAAGCAGTTCGACACCTACAACCTGTGGAACACCTTCACCAGGCTGCAGA | |
| | GCCTGGAGAACGTGGCCTTCAACGTGGTGAACAAGGGCCACTTCGACGGCCAG | |
| | CAGGGCGAGGTGCCCGTGAGCATCATCAACAACACCGTGTACACCAAGGTGGAC | |
| | GGCGTGGACGTGGAGCTGTTCGAGAACAAGACCACCCTGCCCGTGAACGTGGC | |
| | CTTCGAGCTGTGGGCCAAGAGGAACATCAAGCCCGTGCCCGAGGTGAAGATCCT | |
| | GAACAACCTGGGCGTGGACATCGCCGCCAACACCGTGATCTGGGACTACAAGAG | |
| | GGACGCCCCCGCCCACATCAGCACCATCGGCGTGTGCAGCATGACCGACATCGC | |
| | CAAGAAGCCCACCGAGACCATCTGCGCCCCCCTGACCGTGTTCTTCGACGGCAG | |
| | GGTGGACGGCCAGGTGGACCTGTTCAGGAACGCCAGGAACGGCGTGCTGATCA | |
| | CCGAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTACTA | |
| | CAAGAAGGTGGACGGCGTGGTGCAGCAGCTGCCCGAGACCTACTTCACCCAGA | |
| | GCAGGAACCTGCAGGAGTTCAAGCCCAGGAGCCAGATGGAGATCGACTTCCTGG | |
| | AGCTGGCCATGGACGAGTTCATCGAGAGGTACAAGCTGGAGGGCTACGCCTTCG | |
| | AGCACATCGTGTACGGCGACTTCAGCCACAGCCAGCTGGGCGGCCTGCACCTGC | |
| | TGATCGGCCTGGCCAAGAGGTTCAAGGAGAGCCCCTTCGAGCTGGAGGACTTCA | |
| | TCCCCATGGACAGCACCGTGAAGAACTACTTCATCACCGACGCCCAGACCGGCA | |
| | GCAGCAAGTGCGTGTGCAGCGTGATCGACCTGCTGCTGGACGACTTCGTGGAGA | |
| | TCATCAAGAGCCAGGACCTGAGCGTGGTGAGCAAGGTGGTGAAGGTGACCATCG | |
| | ACTACACCGAGATCAGCTTCATGCTGTGGTGCAAGGACGGCCACGTGGAGACCT | |
| | TCTACCCCAAGCTGCAGAGCAGCCAGGCCTGGCAGCCCGGCGTGGCCATGCCC | |
| | AACCTGTACAAGATGCAGAGGATGCTGCTGGAGAAGTGCGACCTGCAGAACTAC | |
| | GGCGACAGCGCCACCCTGCCCAAGGGCATCATGATGAACGTGGCCAAGTACACC | |
| | CAGCTGTGCCAGTACCTGAACACCCTGACCCTGGCCGTGCCCTACAACATGAGG | |
| | GTGATCCACTTCGGCGCCGGCAGCGACAAGGGCGTGGCCCCCGGCACCGCCGT | |
| | GCTGAGGCAGTGGCTGCCCACCGGCACCCTGCTGGTGGACAGCGACCTGAACG | |
| | ACTTCGTGAGCGACGCCGACAGCACCCTGATCGGCGACTGCGCCACCGTGCACA | |
| | CCGCCAACAAGTGGGACCTGATCATCAGCGACATGTACGACCCCAAGACCAAGAA | |
| | CGTGACCAAGGAGAACGACAGCAAGGAGGGCTTCTTCACCTACATCTGCGGCTT | |
| | CATCCAGCAGAAGCTGGCCCTGGGCGGCAGCGTGGCCATCAAGATCACCGAGCA | |
| | CAGCTGGAACGCCGACCTGTACAAGCTGATGGGCCACTTCGCCTGGTGGACCGC | |
| | CTTCGTGACCAACGTGAACGCCAGCAGCAGCGAGGCCTTCCTGATCGGCTGCAA | |
| | CTACCTGGGCAAGCCCAGGGAGCAGATCGACGGCTACGTGATGCACGCCAACTA | |
| | CATCTTCTGGAGGAACACCAACCCCATCCAGCTGAGCAGCTACAGCCTGTTCGAC | |
| | ATGAGCAAGTTCCCCCTGAAGCT | |
| Envelope | ATGTACAGCTTCGTGAGCGAGGAGACCGGCACCCTGATCGTGAACAGCGTGCTG | 61 |
| | CTGTTCCTGGCCTTCGTGGTGTTCCTGCTGGTGACCCTGGCCATCCTGACCGCC | |
| | CTGAGGCTGTGCGCCTACTGCTGCAACATCGTGAACGTGAGCCTGGTGAAGCCC | |
| | AGCTTCTACGTGTACAGCAGGGTGAAGAACCTGAACAGCAGCAGGGTGCCCGAC | |
| | CTGCTGGTG | |
| Membrane | GCCGACAGCAACGGCACCATCACCGTGGAGGAGCTGAAGAAGCTGCTGGAGCA | 62 |
| | GTGGAACCTGGTGATCGGCTTCCTGTTCCTGACCTGGATCTGCCTGCTGCAGTTC | |
| | GCCTACGCCAACAGGAACAGGTTCCTGTACATCATCAAGCTGATCTTCCTGTGGC | |
| | TGCTGTGGCCCGTGACCCTGGCCTGCTTCGTGCTGGCCGCCGTGTACAGGATCA | |
| | ACTGGATCACCGGCGGCATCGCCATCGCCATGGCCTGCCTGGTGGGCCTGATGT | |
| | GGCTGAGCTACTTCATCGCCAGCTTCAGGCTGTTCGCCAGGACCAGGAGCATGT | |
| | GGAGCTTCAACCCCGAGACCAACATCCTGCTGAACGTGCCCCTGCACGGCACCA | |
| | TCCTGACCAGGCCCCTGCTGGAGAGCGAGCTGGTGATCGGCGCCGTGATCCTGA | |
| | GGGGCCACCTGAGGATCGCCGGCCACCACCTGGGCAGGTGCGACATCAAGGAC | |
| | CTGCCCAAGGAGATCACCGTGGCCACCAGCAGGACCCTGAGCTACTACAAGCTG | |
| | GGCGCCAGCCAGAGGGTGGCCGGCGACAGCGGCTTCGCCGCCTACAGCAGGTA | |
| | CAGGATCGGCAACTACAAGCTGAACACCGACCACAGCAGCAGCAGCGACAACAT | |
| | CGCCCTGCTGGTGCAG | |
| Orf7a | AAGATCATCCTGTTCCTGGCCCTGATCACCCTGGCCACCTGCGAGCTGTACCACT | 63 |
| | ACCAGGAGTGCGTGAGGGGCACCACCGTG | |

In some embodiments, the one or more of the large sequences comprises a T-cell epitope restricted to a large number of human class 1 and class 2 HLA haplotypes and are not restricted to HLA-0201 for class 1 or HLA-DR for class 2.

In some embodiments, the one or more large sequences are separated by a linker. The linker may be 2 to 10 amino acids in length.

In some embodiments, the one or more large sequences are operatively linked to a generic promoter. In some embodiments, the generic promoter is a CMV or a CAG promoter.

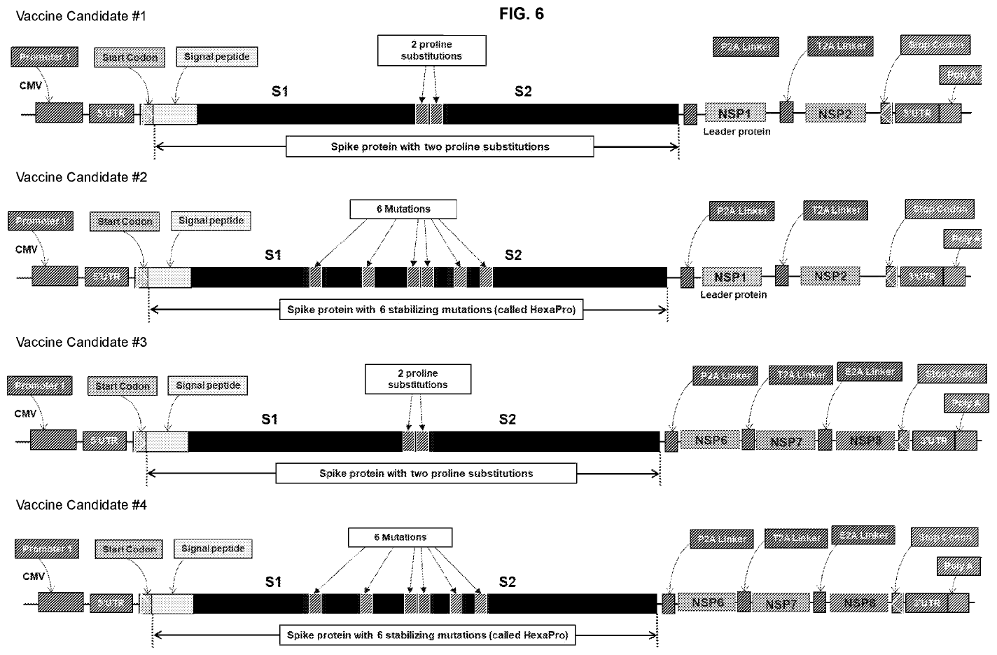

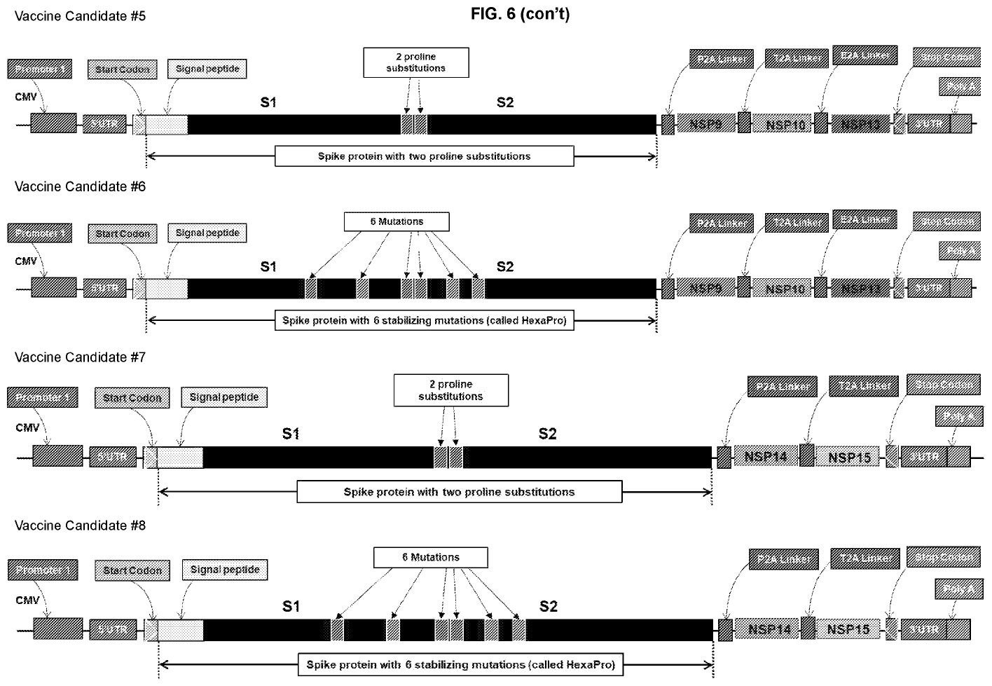

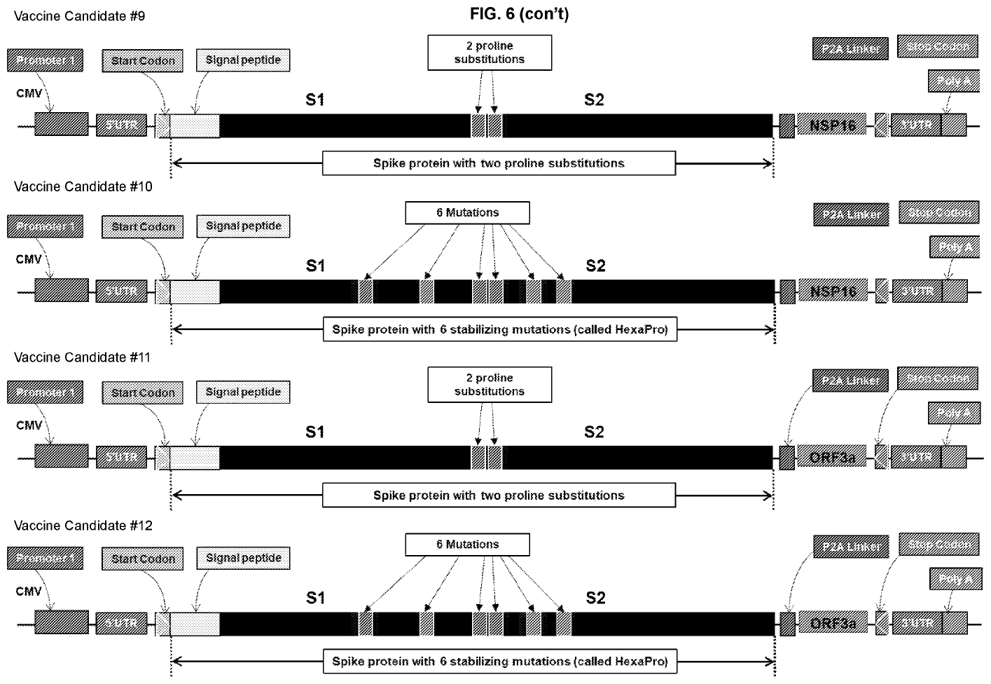

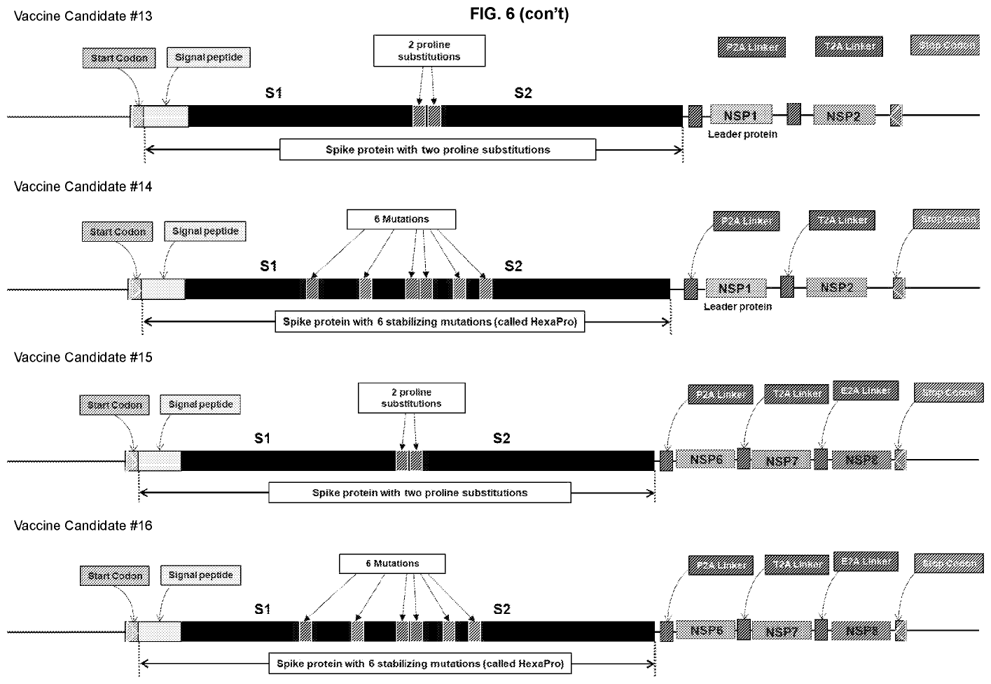

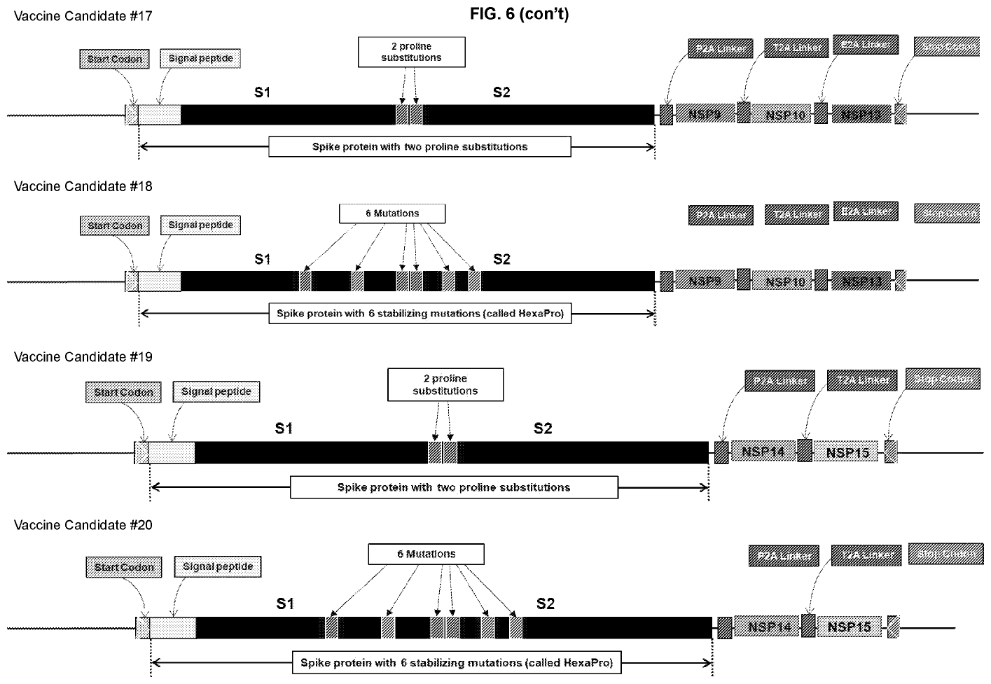

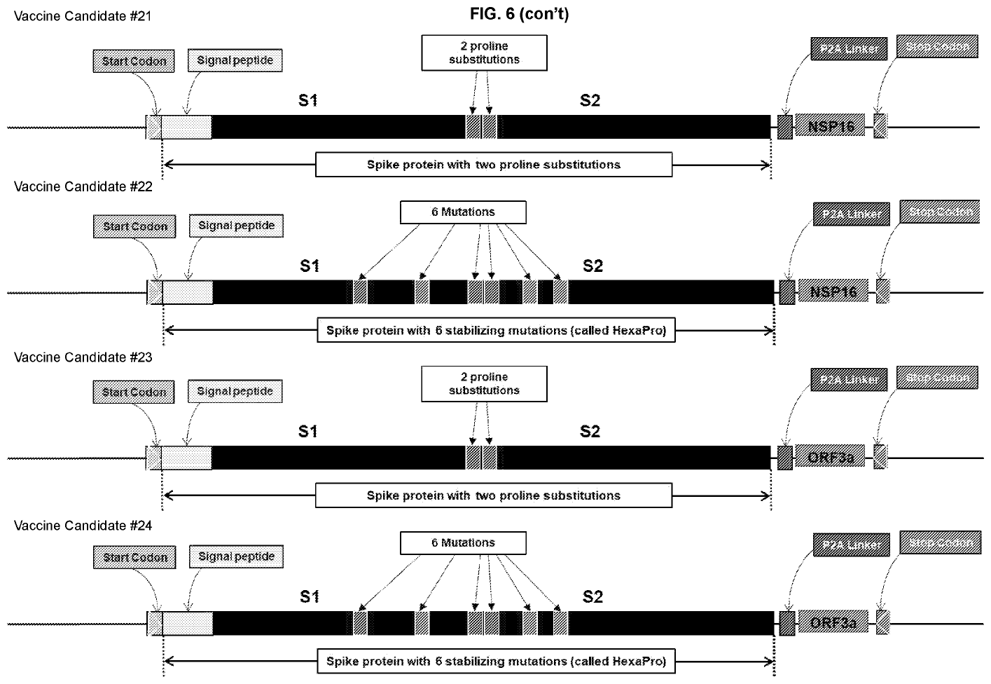

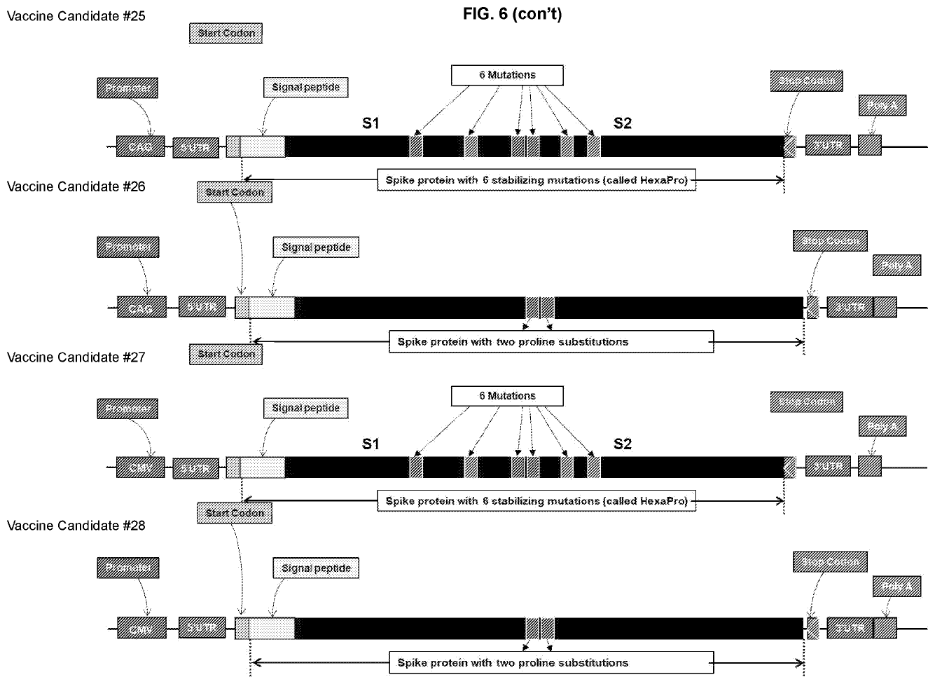

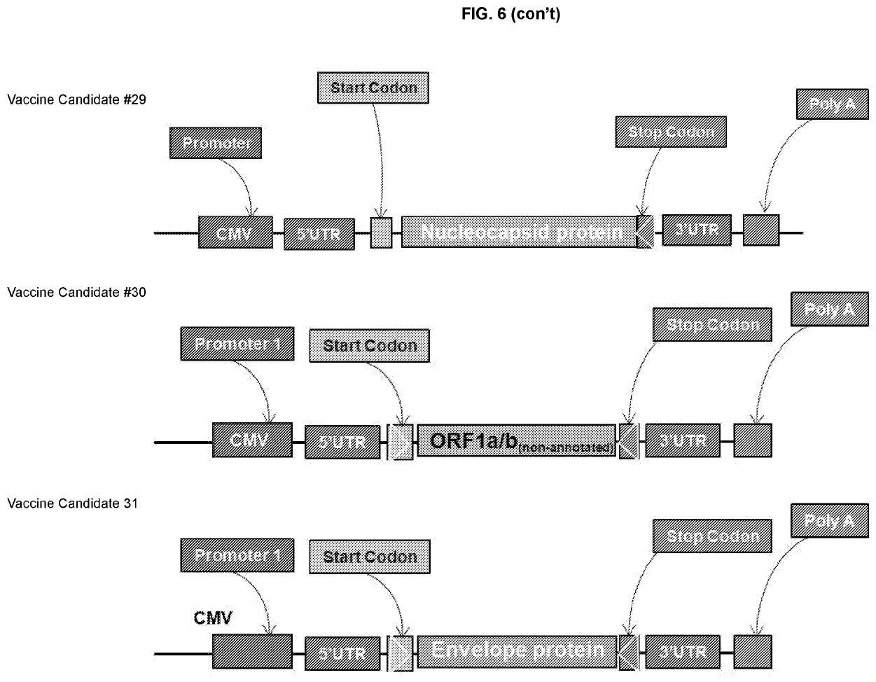

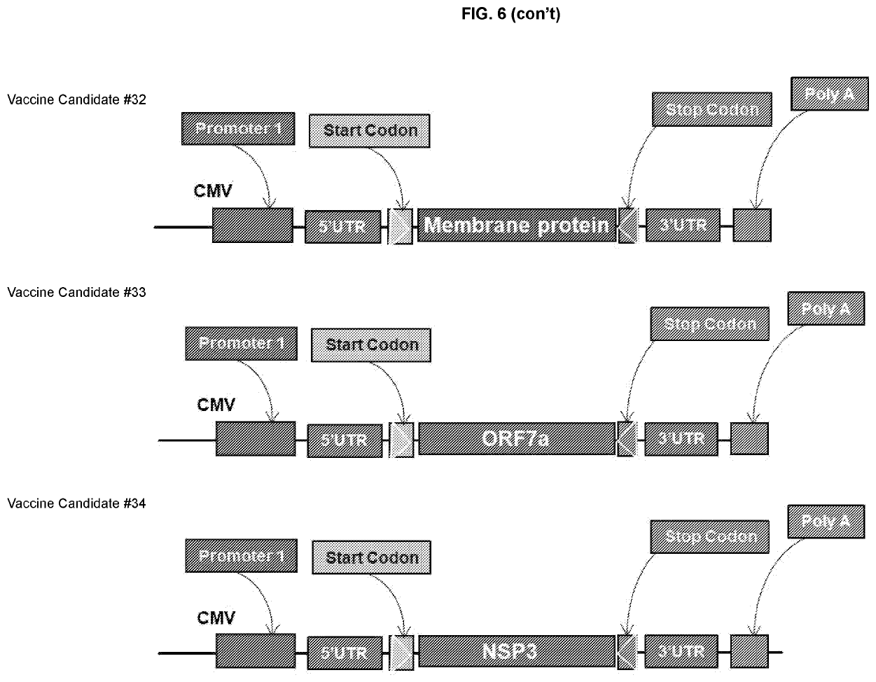

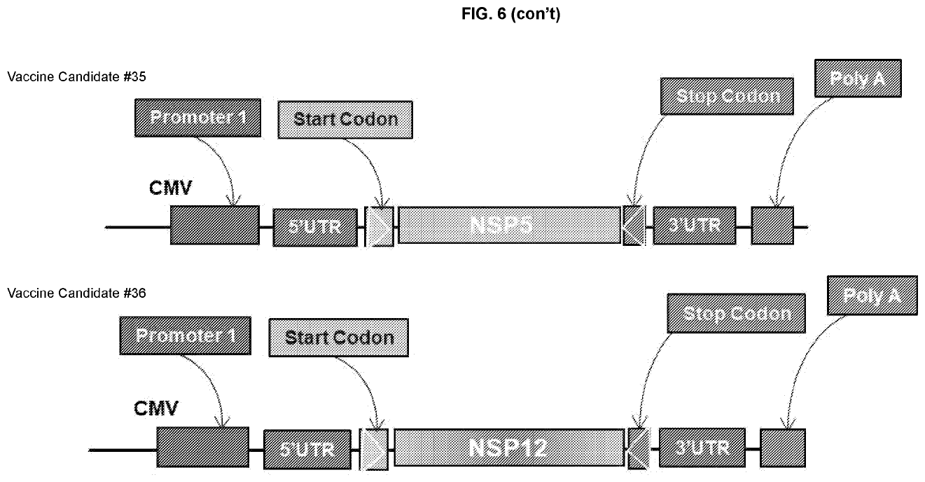

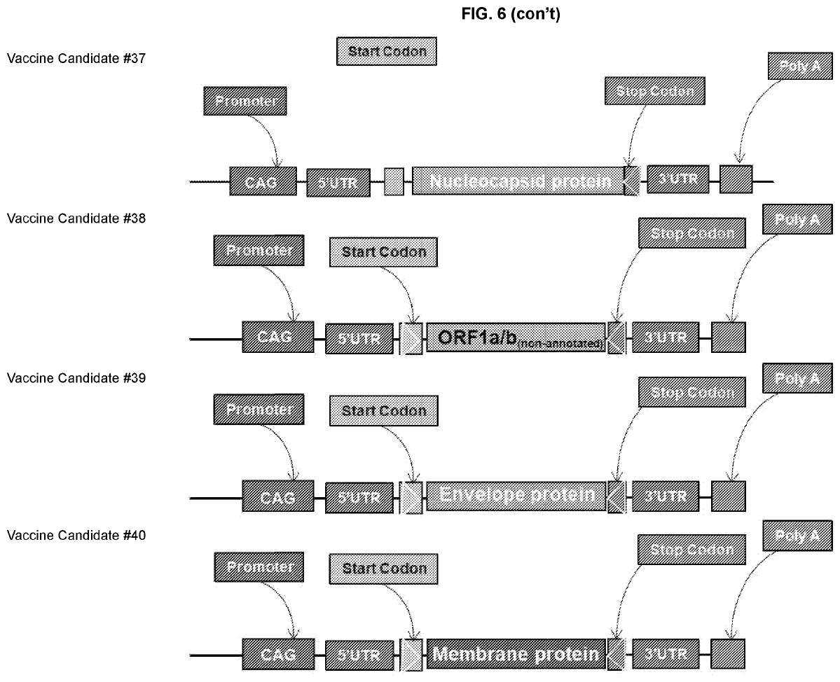

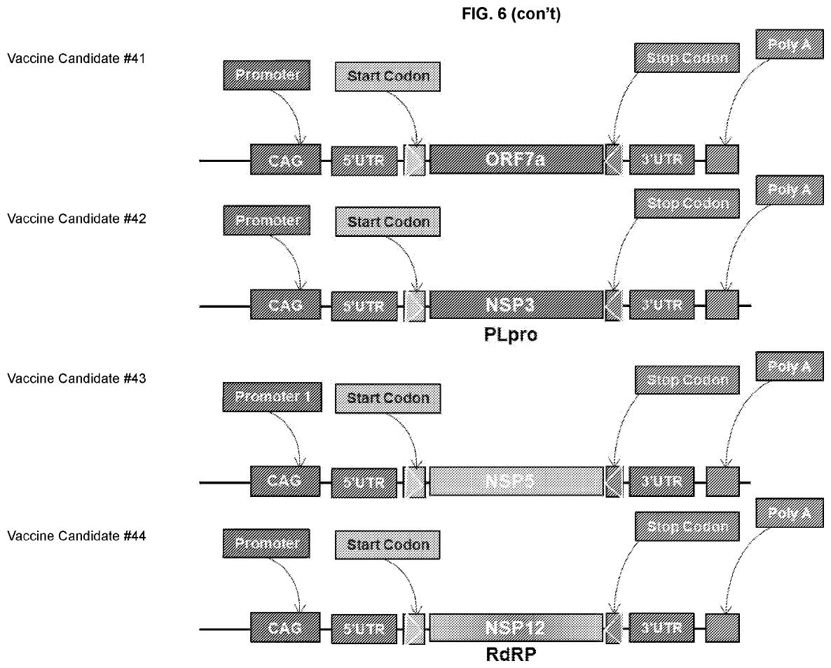

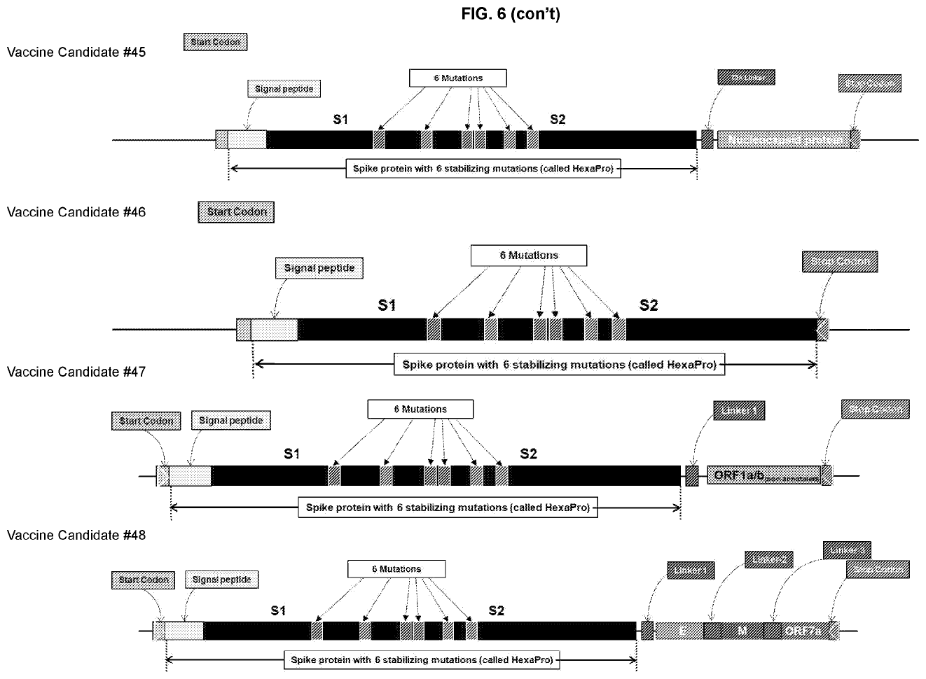

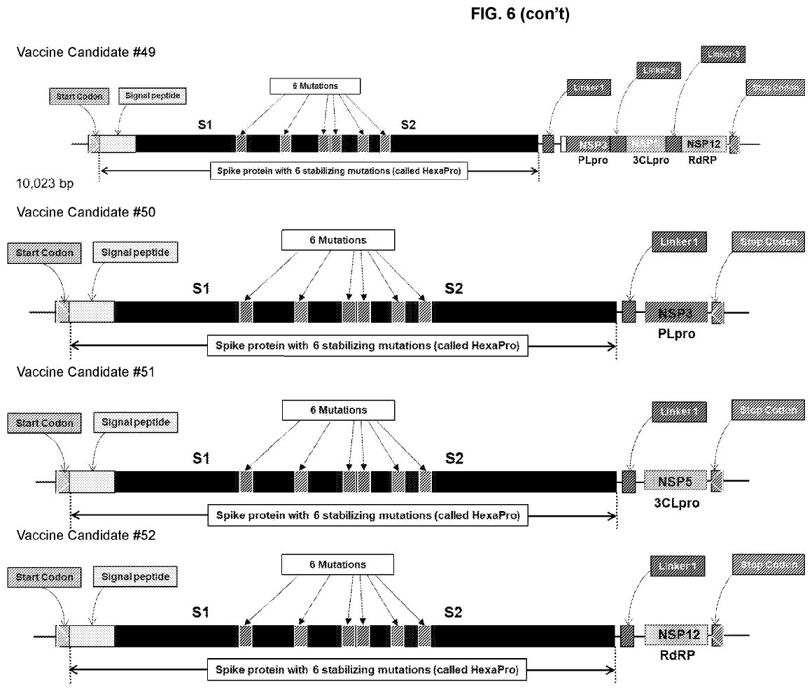

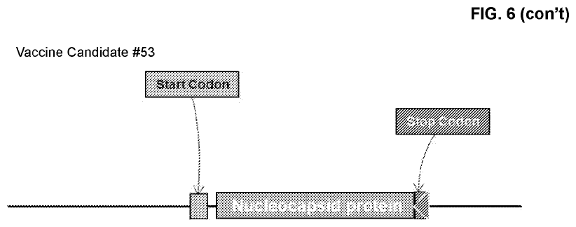

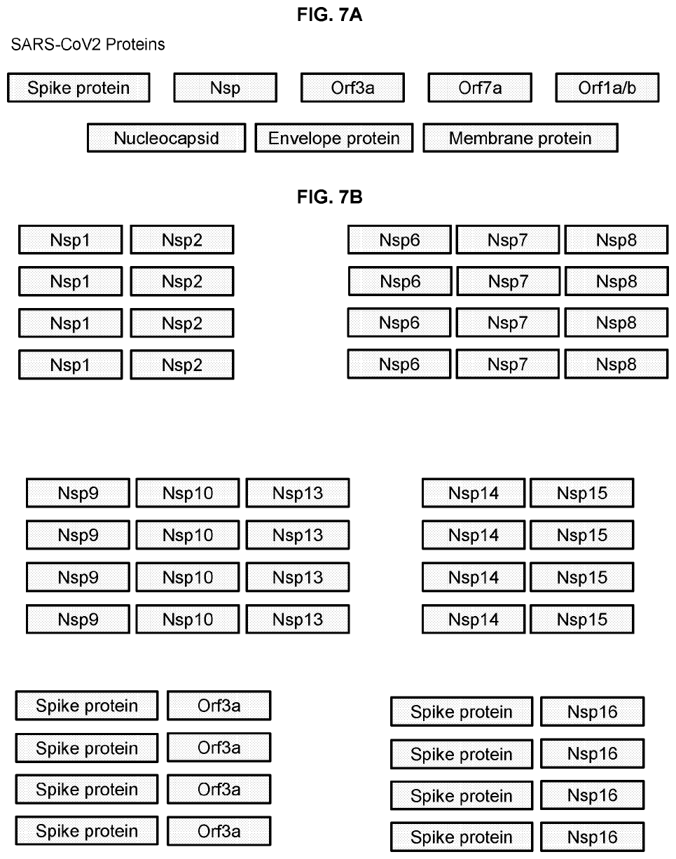

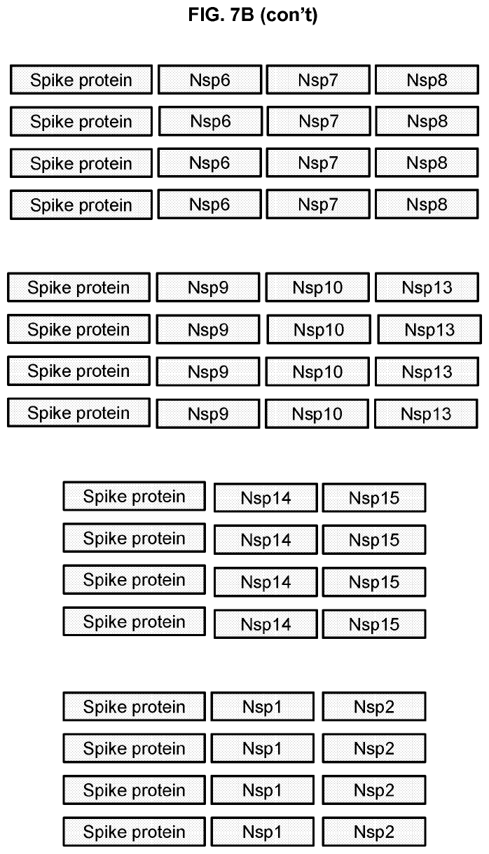

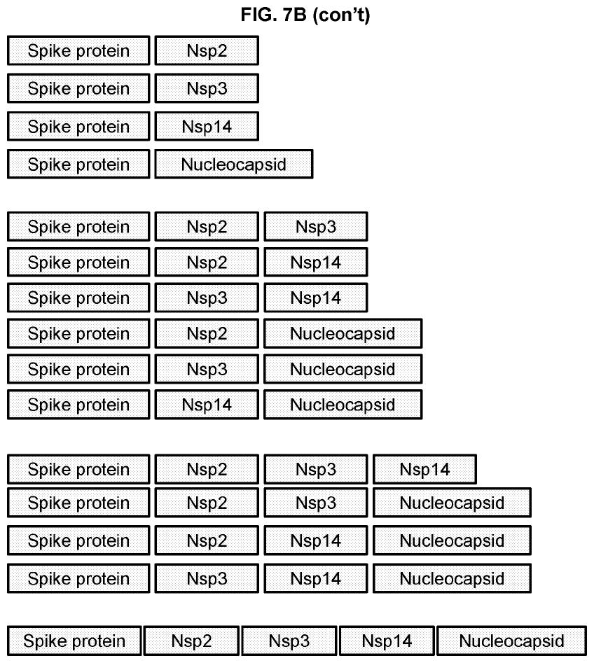

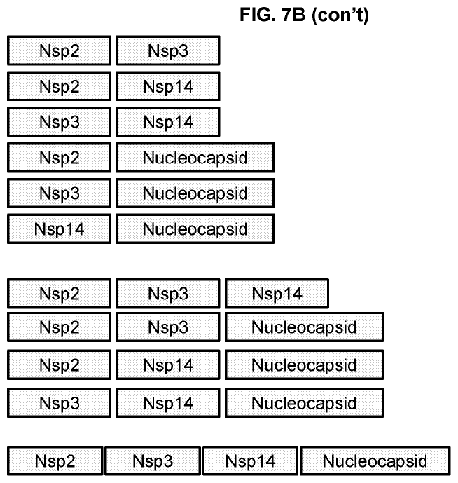

Table 6 and FIG. 6 shows examples of vaccine compositions described herein. The present Invention is not limited to the examples in Table 6.

| | | SEQ |
|---|---|---|
| Vaccine | | ID |
| Candidate | Sequence | NO: |
| Ad5-Pan- | TAGTTATTAATAGTAATCAATTACGGGGTCATTAGTTCATAGCCCATATATGGAGT | 64 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #1 | ACTTTCCATTGACGTCAATGGGTGGAGTATTTACGGTAAACTGCCCACTTGGCA | |
| Spike-NS | GTACATCAAGTGTATCATATGCCAAGTACGCCCCCTATTGACGTCAATGACGGT | |
| P1-NSP2 | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | GAGCCTGGTGCCCGGCTTCAACGAGAAGACCCACGTGCAGCTGAGCCTGCC | |
| | CGTGCTGCAGGTGAGGGACGTGCTGGTGAGGGGCTTCGGCGACAGCGTGGA | |
| | GGAGGTGCTGAGCGAGGCCAGGCAGCACCTGAAGGACGGCACCTGCGGCCT | |
| | GGTGGAGGTGGAGAAGGGCGTGCTGCCCCAGCTGGAGCAGCCCTACGTGTT | |
| | CATCAAGAGGAGCGACGCCAGGACCGCCCCCCACGGCCACGTGATGGTGGA | |
| | GCTGGTGGCCGAGCTGGAGGGCATCCAGTACGGCAGGAGCGGCGAGACCCT | |
| | GGGCGTGCTGGTGCCCCACGTGGGCGAGATCCCCGTGGCCTACAGGAAGGT | |
| | GCTGCTGAGGAAGAACGGCAACAAGGGCGCCGGCGGCCACAGCTACGGCGC | |
| | CGACCTGAAGAGCTTCGACCTGGGCGACGAGCTGGGCACCGACCCCTACGA | |
| | GGACTTCCAGGAGAACTGGAACACCAAGCACAGCAGCGGCGTGACCAGGGA | |
| | GCTGATGAGGGAGCTGAACGGCGGCGGAAGCGGAGAGGGCAGGGGAAGTCT | |
| | TCTAACATGCGGGGACGTGGAGGAAAATCCCGGCCCCGCCTACACCAGGTAC | |
| | GTGGACAACAACTTCTGCGGCCCCGACGGCTACCCCCTGGAGTGCATCAAGG | |
| | ACCTGCTGGCCAGGGCCGGCAAGGCCAGCTGCACCCTGAGCGAGCAGCTGG | |
| | ACTTCATCGACACCAAGAGGGGCGTGTACTGCTGCAGGGAGCACGAGCACGA | |
| | GATCGCCTGGTACACCGAGAGGAGCGAGAAGAGCTACGAGCTGCAGACCCCC | |
| | TTCGAGATCAAGCTGGCCAAGAAGTTCGACACCTTCAACGGCGAGTGCCCCA | |
| | ACTTCGTGTTCCCCCTGAACAGCATCATCAAGACCATCCAGCCCAGGGTGGAG | |
| | AAGAAGAAGCTGGACGGCTTCATGGGCAGGATCAGGAGCGTGTACCCCGTGG | |
| | CCAGCCCCAACGAGTGCAACCAGATGTGCCTGAGCACCCTGATGAAGTGCGA | |
| | CCACTGCGGCGAGACCAGCTGGCAGACCGGCGACTTCGTGAAGGCCACCTG | |
| | CGAGTTCTGCGGCACCGAGAACCTGACCAAGGAGGGCGCCACCACCTGCGG | |
| | CTACCTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAGCGGC | |
| | CTGAAGACCATCCTGAGGAAGGGCGGCAGGACCATCGCCTTCGGCGGCTGC | |
| | GTGTTCAGCTACGTGGGCTGCCACAACAAGTGCGCCTACTGGGTGCCCAGGG | |
| | CCAGCGCCAACATCGGCTGCAACCACACCGGCGTGGTGGGCGAGGGCAGCG | |
| | AGGGCCTGAACGACAACCTGCTGGAGATCCTGCAGAAGGAGAAGGTGAACAT | |
| | CAACATCGTGGGCGACTTCAAGCTGAACGAGGAGATCGCCATCATCCTGGCCA | |
| | GCTTCAGCGCCAGCACCAGCGCCTTCGTGGAGACCGTGAAGGGCCTGGACTA | |
| | CAAGGCCTTCAAGCAGATCGTGGAGAGCTGCGGCAACTTCAAGGTGACCAAG | |
| | GGCAAGGCCAAGAAGGGCGCCTGGAACATCGGCGAGCAGAAGAGCATCCTG | |
| | AGCCCCCTGTACGCCTTCGCCAGCGAGGCCGCCAGGGTGGTGAGGAGCATC | |
| | TTCAGCAGGACCCTGGAGACCGCCCAGAACAGCGTGAGGGTGCTGCAGAAG | |
| | GCCGCCATCACCATCCTGGACGGCATCAGCCAGTACAGCCTGAGGCTGATCG | |
| | ACGCCATGATGTTCACCAGCGACCTGGCCACCAACAACCTGGTGGTGATGGC | |
| | CTACATCACCGGCGGCGTGGTGCAGCTGACCAGCCAGTGGCTGACCAACATC | |
| | TTCGGCACCGTGTACGAGAAGCTGAAGCCCGTGCTGGACTGGCTGGAGGAGA | |
| | AGTTCAAGGAGGGCGTGGAGTTCCTGAGGGACGGCTGGGAGATCGTGAAGTT | |
| | CATCAGCACCTGCGCCTGCGAGATCGTGGGCGGCCAGATCGTGACCTGCGCC | |
| | AAGGAGATCAAGGAGAGCGTGCAGACCTTCTTCAAGCTGGTGAACAAGTTCCT | |
| | GGCCCTGTGCGCCGACAGCATCATCATCGGCGGCGCCAAGCTGAAGGCCCT | |
| | GAACCTGGGCGAGACCTTCGTGACCCACAGCAAGGGCCTGTACAGGAAGTGC | |
| | GTGAAGAGCAGGGAGGAGACCGGCCTGCTGATGCCCCTGAAGGCCCCCAAG | |
| | GAGATCATCTTCCTGGAGGGCGAGACCCTGCCCACCGAGGTGCTGACCGAGG | |
| | AGGTGGTGCTGAAGACCGGCGACCTGCAGCCCCTGGAGCAGCCCACCAGCG | |
| | AGGCCGTGGAGGCCCCCCTGGTGGGCACCCCCGTGTGCATCAACGGCCTGA | |
| | TGCTGCTGGAGATCAAGGACACCGAGAAGTACTGCGCCCTGGCCCCCAACAT | |
| | GATGGTGACCAACAACACCTTCACCCTGAAGGGCGGCTGATGACTCGAGCTG | |
| | GTACTGCATGCACGCAATGCTAGCTGCCCCTTTCCCGTCCTGGGTACCCCGAG | |
| | TCTCCCCCGACCTCGGGTCCCAGGTATGCTCCCACCTCCACCTGCCCCACTC | |
| | ACCACCTCTGCTAGTTCCAGACACCTCCCAAGCACGCAGCAATGCAGCTCAAA | |
| | ACGCTTAGCCTAGCCACACCCCCACGGGAAACAGCAGTGATTAACCTTTAGCA | |
| | ATAAACGAAAGTTTAACTAAGCTATACTAACCCCAGGGTTGGTCAATTTCGTGCC | |
| | AGCCACACCCTGGAGCTAGCAAAAAAAA | |
| Ad5-Pan- | TAGTTATTAATAGTAATCAATTACGGGGTCATTAGTTCATAGCCCATATATGGAGT | 65 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #2 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| NSP1-NS | AAATGGCCCGCCTGGCATTATGCCCAGTACATGACCTTATGGGACTTTCCTACT | |
| P2 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | GAGCCTGGTGCCCGGCTTCAACGAGAAGACCCACGTGCAGCTGAGCCTGCC | |
| | CGTGCTGCAGGTGAGGGACGTGCTGGTGAGGGGCTTCGGCGACAGCGTGGA | |
| | GGAGGTGCTGAGCGAGGCCAGGCAGCACCTGAAGGACGGCACCTGCGGCCT | |
| | GGTGGAGGTGGAGAAGGGCGTGCTGCCCCAGCTGGAGCAGCCCTACGTGTT | |
| | CATCAAGAGGAGCGACGCCAGGACCGCCCCCCACGGCCACGTGATGGTGGA | |
| | GCTGGTGGCCGAGCTGGAGGGCATCCAGTACGGCAGGAGCGGCGAGACCCT | |
| | GGGCGTGCTGGTGCCCCACGTGGGCGAGATCCCCGTGGCCTACAGGAAGGT | |
| | GCTGCTGAGGAAGAACGGCAACAAGGGCGCCGGCGGCCACAGCTACGGCGC | |
| | CGACCTGAAGAGCTTCGACCTGGGCGACGAGCTGGGCACCGACCCCTACGA | |
| | GGACTTCCAGGAGAACTGGAACACCAAGCACAGCAGCGGCGTGACCAGGGA | |
| | GCTGATGAGGGAGCTGAACGGCGGCGGAAGCGGAGAGGGCAGGGGAAGTCT | |
| | TCTAACATGCGGGGACGTGGAGGAAAATCCCGGCCCCGCCTACACCAGGTAC | |
| | GTGGACAACAACTTCTGCGGCCCCGACGGCTACCCCCTGGAGTGCATCAAGG | |
| | ACCTGCTGGCCAGGGCCGGCAAGGCCAGCTGCACCCTGAGCGAGCAGCTGG | |
| | ACTTCATCGACACCAAGAGGGGCGTGTACTGCTGCAGGGAGCACGAGCACGA | |
| | GATCGCCTGGTACACCGAGAGGAGCGAGAAGAGCTACGAGCTGCAGACCCCC | |
| | TTCGAGATCAAGCTGGCCAAGAAGTTCGACACCTTCAACGGCGAGTGCCCCA | |
| | ACTTCGTGTTCCCCCTGAACAGCATCATCAAGACCATCCAGCCCAGGGTGGAG | |
| | AAGAAGAAGCTGGACGGCTTCATGGGCAGGATCAGGAGCGTGTACCCCGTGG | |
| | CCAGCCCCAACGAGTGCAACCAGATGTGCCTGAGCACCCTGATGAAGTGCGA | |
| | CCACTGCGGCGAGACCAGCTGGCAGACCGGCGACTTCGTGAAGGCCACCTG | |
| | CGAGTTCTGCGGCACCGAGAACCTGACCAAGGAGGGCGCCACCACCTGCGG | |
| | CTACCTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAGCGGC | |
| | CTGAAGACCATCCTGAGGAAGGGCGGCAGGACCATCGCCTTCGGCGGCTGC | |
| | GTGTTCAGCTACGTGGGCTGCCACAACAAGTGCGCCTACTGGGTGCCCAGGG | |
| | CCAGCGCCAACATCGGCTGCAACCACACCGGCGTGGTGGGCGAGGGCAGCG | |
| | AGGGCCTGAACGACAACCTGCTGGAGATCCTGCAGAAGGAGAAGGTGAACAT | |
| | CAACATCGTGGGCGACTTCAAGCTGAACGAGGAGATCGCCATCATCCTGGCCA | |
| | GCTTCAGCGCCAGCACCAGCGCCTTCGTGGAGACCGTGAAGGGCCTGGACTA | |
| | CAAGGCCTTCAAGCAGATCGTGGAGAGCTGCGGCAACTTCAAGGTGACCAAG | |
| | GGCAAGGCCAAGAAGGGCGCCTGGAACATCGGCGAGCAGAAGAGCATCCTG | |
| | AGCCCCCTGTACGCCTTCGCCAGCGAGGCCGCCAGGGTGGTGAGGAGCATC | |
| | TTCAGCAGGACCCTGGAGACCGCCCAGAACAGCGTGAGGGTGCTGCAGAAG | |
| | GCCGCCATCACCATCCTGGACGGCATCAGCCAGTACAGCCTGAGGCTGATCG | |
| | ACGCCATGATGTTCACCAGCGACCTGGCCACCAACAACCTGGTGGTGATGGC | |
| | CTACATCACCGGCGGCGTGGTGCAGCTGACCAGCCAGTGGCTGACCAACATC | |
| | TTCGGCACCGTGTACGAGAAGCTGAAGCCCGTGCTGGACTGGCTGGAGGAGA | |
| | AGTTCAAGGAGGGCGTGGAGTTCCTGAGGGACGGCTGGGAGATCGTGAAGTT | |
| | CATCAGCACCTGCGCCTGCGAGATCGTGGGCGGCCAGATCGTGACCTGCGCC | |
| | AAGGAGATCAAGGAGAGCGTGCAGACCTTCTTCAAGCTGGTGAACAAGTTCCT | |
| | GGCCCTGTGCGCCGACAGCATCATCATCGGCGGCGCCAAGCTGAAGGCCCT | |
| | GAACCTGGGCGAGACCTTCGTGACCCACAGCAAGGGCCTGTACAGGAAGTGC | |
| | GTGAAGAGCAGGGAGGAGACCGGCCTGCTGATGCCCCTGAAGGCCCCCAAG | |
| | GAGATCATCTTCCTGGAGGGCGAGACCCTGCCCACCGAGGTGCTGACCGAGG | |
| | AGGTGGTGCTGAAGACCGGCGACCTGCAGCCCCTGGAGCAGCCCACCAGCG | |
| | AGGCCGTGGAGGCCCCCCTGGTGGGCACCCCCGTGTGCATCAACGGCCTGA | |
| | TGCTGCTGGAGATCAAGGACACCGAGAAGTACTGCGCCCTGGCCCCCAACAT | |
| | GATGGTGACCAACAACACCTTCACCCTGAAGGGCGGCTGATGACTCGAGCTG | |
| | GTACTGCATGCACGCAATGCTAGCTGCCCCTTTCCCGTCCTGGGTACCCCGAG | |
| | TCTCCCCCGACCTCGGGTCCCAGGTATGCTCCCACCTCCACCTGCCCCACTC | |
| | ACCACCTCTGCTAGTTCCAGACACCTCCCAAGCACGCAGCAATGCAGCTCAAA | |
| | ACGCTTAGCCTAGCCACACCCCCACGGGAAACAGCAGTGATTAACCTTTAGCA | |
| | ATAAACGAAAGTTTAACTAAGCTATACTAACCCCAGGGTTGGTCAATTTCGTGCC | |
| | AGCCACACCCTGGAGCTAGCAAAAAAAA | |
| Ad5-Pan- | TAGTTATTAATAGTAATCAATTACGGGGTCATTAGTTCATAGCCCATATATGGAGT | 66 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #3 | ACTTTCCATTGACGTCAATGGGTGGAGTATTTACGGTAAACTGCCCACTTGGCA | |
| Spike-NS | GTACATCAAGTGTATCATATGCCAAGTACGCCCCCTATTGACGTCAATGACGGT | |
| P6-NSP7- | AAATGGCCCGCCTGGCATTATGCCCAGTACATGACCTTATGGGACTTTCCTACT | |
| NSP8 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGC | |
| | CGTGAAGAGGACCATCAAGGGCACCCACCACTGGCTGCTGCTGACCATCCTG | |
| | ACCAGCCTGCTGGTGCTGGTGCAGAGCACCCAGTGGAGCCTGTTCTTCTTCC | |
| | TGTACGAGAACGCCTTCCTGCCCTTCGCCATGGGCATCATCGCCATGAGCGCC | |
| | TTCGCCATGATGTTCGTGAAGCACAAGCACGCCTTCCTGTGCCTGTTCCTGCT | |
| | GCCCAGCCTGGCCACCGTGGCCTACTTCAACATGGTGTACATGCCCGCCAGC | |
| | TGGGTGATGAGGATCATGACCTGGCTGGACATGGTGGACACCAGCCTGAGCG | |
| | GCTTCAAGCTGAAGGACTGCGTGATGTACGCCAGCGCCGTGGTGCTGCTGAT | |
| | CCTGATGACCGCCAGGACCGTGTACGACGACGGCGCCAGGAGGGTGTGGAC | |
| | CCTGATGAACGTGCTGACCCTGGTGTACAAGGTGTACTACGGCAACGCCCTG | |
| | GACCAGGCCATCAGCATGTGGGCCCTGATCATCAGCGTGACCAGCAACTACA | |
| | GCGGCGTGGTGACCACCGTGATGTTCCTGGCCAGGGGCATCGTGTTCATGTG | |
| | CGTGGAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCTGTTC | |
| | TGCCTGCTGAACAGGTACTTCAGGCTGACCCTGGGCGTGTACGACTACCTGG | |
| | TGAGCACCCAGGAGTTCAGGTACATGAACAGCCAGGGCCTGCTGCCCCCCAA | |
| | GAACAGCATCGACGCCTTCAAGCTGAACATCAAGCTGCTGGGCGTGGGCGGC | |
| | AAGCCCTGCATCAAGGTGGCCACCGTGCAGGGAAGCGGAGAGGGCAGGGGA | |
| | AGTCTTCTAACATGCGGGGACGTGGAGGAAAATCCCGGCCCCAGCAAGATGA | |
| | GCGACGTGAAGTGCACCAGCGTGGTGCTGCTGAGCGTGCTGCAGCAGCTGA | |
| | GGGTGGAGAGCAGCAGCAAGCTGTGGGCCCAGTGCGTGCAGCTGCACAACG | |
| | ACATCCTGCTGGCCAAGGACACCACCGAGGCCTTCGAGAAGATGGTGAGCCT | |
| | GCTGAGCGTGCTGCTGAGCATGCAGGGCGCCGTGGACATCAACAAGCTGTGC | |
| | GAGGAGATGCTGGACAACAGGGCCACCCTGCAGGGAAGCGGACAGTGTACTA | |
| | ATTATGCTCTCTTGAAATTGGCTGGAGATGTTGAGAGCAACCCAGGTCCCGCC | |
| | ATCGCCAGCGAGTTCAGCAGCCTGCCCAGCTACGCCGCCTTCGCCACCGCCC | |
| | AGGAGGCCTACGAGCAGGCCGTGGCCAACGGCGACAGCGAGGTGGTGCTGA | |
| | AGAAGCTGAAGAAGAGCCTGAACGTGGCCAAGAGCGAGTTCGACAGGGACG | |
| | CCGCCATGCAGAGGAAGCTGGAGAAGATGGCCGACCAGGCCATGACCCAGAT | |
| | GTACAAGCAGGCCAGGAGCGAGGACAAGAGGGCCAAGGTGACCAGCGCCAT | |
| | GCAGACCATGCTGTTCACCATGCTGAGGAAGCTGGACAACGACGCCCTGAAC | |
| | AACATCATCAACAACGCCAGGGACGGCTGCGTGCCCCTGAACATCATCCCCCT | |
| | GACCACCGCCGCCAAGCTGATGGTGGTGATCCCCGACTACAACACCTACAAG | |
| | AACACCTGCGACGGCACCACCTTCACCTACGCCAGCGCCCTGTGGGAGATCC | |
| | AGCAGGTGGTGGACGCCGACAGCAAGATCGTGCAGCTGAGCGAGATCAGCAT | |
| | GGACAACAGCCCCAACCTGGCCTGGCCCCTGATCGTGACCGCCCTGAGGGC | |
| | CAACAGCGCCGTGAAGCTGCAGTGATGACTCGAGCTGGTACTGCATGCACGC | |
| | AATGCTAGCTGCCCCTTTCCCGTCCTGGGTACCCCGAGTCTCCCCCGACCTC | |
| | GGGTCCCAGGTATGCTCCCACCTCCACCTGCCCCACTCACCACCTCTGCTAGT | |
| | TCCAGACACCTCCCAAGCACGCAGCAATGCAGCTCAAAACGCTTAGCCTAGCC | |
| | ACACCCCCACGGGAAACAGCAGTGATTAACCTTTAGCAATAAACGAAAGTTTAA | |
| | CTAAGCTATACTAACCCCAGGGTTGGTCAATTTCGTGCCAGCCACACCCTGGA | |
| | GCTAGCAAAAAAAA | |
| Ad5-Pan- | TAGTTATTAATAGTAATCAATTACGGGGTCATTAGTTCATAGCCCATATATGGAGT | 67 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #4 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| NSP6-NS | AAATGGCCCGCCTGGCATTATGCCCAGTACATGACCTTATGGGACTTTCCTACT | |
| P7-NSP8 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGC | |
| | CGTGAAGAGGACCATCAAGGGCACCCACCACTGGCTGCTGCTGACCATCCTG | |
| | ACCAGCCTGCTGGTGCTGGTGCAGAGCACCCAGTGGAGCCTGTTCTTCTTCC | |
| | TGTACGAGAACGCCTTCCTGCCCTTCGCCATGGGCATCATCGCCATGAGCGCC | |
| | TTCGCCATGATGTTCGTGAAGCACAAGCACGCCTTCCTGTGCCTGTTCCTGCT | |
| | GCCCAGCCTGGCCACCGTGGCCTACTTCAACATGGTGTACATGCCCGCCAGC | |
| | TGGGTGATGAGGATCATGACCTGGCTGGACATGGTGGACACCAGCCTGAGCG | |
| | GCTTCAAGCTGAAGGACTGCGTGATGTACGCCAGCGCCGTGGTGCTGCTGAT | |
| | CCTGATGACCGCCAGGACCGTGTACGACGACGGCGCCAGGAGGGTGTGGAC | |
| | CCTGATGAACGTGCTGACCCTGGTGTACAAGGTGTACTACGGCAACGCCCTG | |
| | GACCAGGCCATCAGCATGTGGGCCCTGATCATCAGCGTGACCAGCAACTACA | |
| | GCGGCGTGGTGACCACCGTGATGTTCCTGGCCAGGGGCATCGTGTTCATGTG | |
| | CGTGGAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCTGTTC | |
| | TGCCTGCTGAACAGGTACTTCAGGCTGACCCTGGGCGTGTACGACTACCTGG | |
| | TGAGCACCCAGGAGTTCAGGTACATGAACAGCCAGGGCCTGCTGCCCCCCAA | |
| | GAACAGCATCGACGCCTTCAAGCTGAACATCAAGCTGCTGGGCGTGGGCGGC | |
| | AAGCCCTGCATCAAGGTGGCCACCGTGCAGGGAAGCGGAGAGGGCAGGGGA | |
| | AGTCTTCTAACATGCGGGGACGTGGAGGAAAATCCCGGCCCCAGCAAGATGA | |
| | GCGACGTGAAGTGCACCAGCGTGGTGCTGCTGAGCGTGCTGCAGCAGCTGA | |
| | GGGTGGAGAGCAGCAGCAAGCTGTGGGCCCAGTGCGTGCAGCTGCACAACG | |
| | ACATCCTGCTGGCCAAGGACACCACCGAGGCCTTCGAGAAGATGGTGAGCCT | |
| | GCTGAGCGTGCTGCTGAGCATGCAGGGCGCCGTGGACATCAACAAGCTGTGC | |
| | GAGGAGATGCTGGACAACAGGGCCACCCTGCAGGGAAGCGGACAGTGTACTA | |
| | ATTATGCTCTCTTGAAATTGGCTGGAGATGTTGAGAGCAACCCAGGTCCCGCC | |
| | ATCGCCAGCGAGTTCAGCAGCCTGCCCAGCTACGCCGCCTTCGCCACCGCCC | |
| | AGGAGGCCTACGAGCAGGCCGTGGCCAACGGCGACAGCGAGGTGGTGCTGA | |
| | AGAAGCTGAAGAAGAGCCTGAACGTGGCCAAGAGCGAGTTCGACAGGGACG | |
| | CCGCCATGCAGAGGAAGCTGGAGAAGATGGCCGACCAGGCCATGACCCAGAT | |
| | GTACAAGCAGGCCAGGAGCGAGGACAAGAGGGCCAAGGTGACCAGCGCCAT | |
| | GCAGACCATGCTGTTCACCATGCTGAGGAAGCTGGACAACGACGCCCTGAAC | |
| | AACATCATCAACAACGCCAGGGACGGCTGCGTGCCCCTGAACATCATCCCCCT | |
| | GACCACCGCCGCCAAGCTGATGGTGGTGATCCCCGACTACAACACCTACAAG | |
| | AACACCTGCGACGGCACCACCTTCACCTACGCCAGCGCCCTGTGGGAGATCC | |
| | AGCAGGTGGTGGACGCCGACAGCAAGATCGTGCAGCTGAGCGAGATCAGCAT | |
| | GGACAACAGCCCCAACCTGGCCTGGCCCCTGATCGTGACCGCCCTGAGGGC | |
| | CAACAGCGCCGTGAAGCTGCAGTGATGACTCGAGCTGGTACTGCATGCACGC | |
| | AATGCTAGCTGCCCCTTTCCCGTCCTGGGTACCCCGAGTCTCCCCCGACCTC | |
| | GGGTCCCAGGTATGCTCCCACCTCCACCTGCCCCACTCACCACCTCTGCTAGT | |
| | TCCAGACACCTCCCAAGCACGCAGCAATGCAGCTCAAAACGCTTAGCCTAGCC | |
| | ACACCCCCACGGGAAACAGCAGTGATTAACCTTTAGCAATAAACGAAAGTTTAA | |
| | CTAAGCTATACTAACCCCAGGGTTGGTCAATTTCGTGCCAGCCACACCCTGGA | |
| | GCTAGCAAAAAAAA | |
| Ad5-Pan- | TAGTTATTAATAGTAATCAATTACGGGGTCATTAGTTCATAGCCCATATATGGAGT | 69 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #5 | ACTTTCCATTGACGTCAATGGGTGGAGTATTTACGGTAAACTGCCCACTTGGCA | |
| Spike-NS | GTACATCAAGTGTATCATATGCCAAGTACGCCCCCTATTGACGTCAATGACGGT | |
| P9-NSP1 | AAATGGCCCGCCTGGCATTATGCCCAGTACATGACCTTATGGGACTTTCCTACT | |
| 0-NSP13 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAACAA | |
| | CGAGCTGAGCCCCGTGGCCCTGAGGCAGATGAGCTGCGCCGCCGGCACCAC | |
| | CCAGACCGCCTGCACCGACGACAACGCCCTGGCCTACTACAACACCACCAAG | |
| | GGCGGCAGGTTCGTGCTGGCCCTGCTGAGCGACCTGCAGGACCTGAAGTGG | |
| | GCCAGGTTCCCCAAGAGCGACGGCACCGGCACCATCTACACCGAGCTGGAG | |
| | CCCCCCTGCAGGTTCGTGACCGACACCCCCAAGGGCCCCAAGGTGAAGTACC | |
| | TGTACTTCATCAAGGGCCTGAACAACCTGAACAGGGGCATGGTGCTGGGCAG | |
| | CCTGGCCGCCACCGTGAGGCTGCAGGGAAGCGGAGAGGGCAGGGGAAGTC | |
| | TTCTAACATGCGGGGACGTGGAGGAAAATCCCGGCCCCGCCGGCAACGCCAC | |
| | CGAGGTGCCCGCCAACAGCACCGTGCTGAGCTTCTGCGCCTTCGCCGTGGA | |
| | CGCCGCCAAGGCCTACAAGGACTACCTGGCCAGCGGCGGCCAGCCCATCAC | |
| | CAACTGCGTGAAGATGCTGTGCACCCACACCGGCACCGGCCAGGCCATCACC | |
| | GTGACCCCCGAGGCCAACATGGACCAGGAGAGCTTCGGCGGCGCCAGCTGC | |
| | TGCCTGTACTGCAGGTGCCACATCGACCACCCCAACCCCAAGGGCTTCTGCG | |
| | ACCTGAAGGGCAAGTACGTGCAGATCCCCACCACCTGCGCCAACGACCCCGT | |
| | GGGCTTCACCCTGAAGAACACCGTGTGCACCGTGTGCGGCATGTGGAAGGGC | |
| | TACGGCTGCAGCTGCGACCAGCTGAGGGAGCCCATGCTGCAGGGAAGCGGA | |
| | CAGTGTACTAATTATGCTCTCTTGAAATTGGCTGGAGATGTTGAGAGCAACCCA | |
| | GGTCCCGCCGTGGGCGCCTGCGTGCTGTGCAACAGCCAGACCAGCCTGAGG | |
| | TGCGGCGCCTGCATCAGGAGGCCCTTCCTGTGCTGCAAGTGCTGCTACGACC | |
| | ACGTGATCAGCACCAGCCACAAGCTGGTGCTGAGCGTGAACCCCTACGTGTG | |
| | CAACGCCCCCGGCTGCGACGTGACCGACGTGACCCAGCTGTACCTGGGCGG | |
| | CATGAGCTACTACTGCAAGAGCCACAAGCCCCCCATCAGCTTCCCCCTGTGCG | |
| | CCAACGGCCAGGTGTTCGGCCTGTACAAGAACACCTGCGTGGGCAGCGACAA | |
| | CGTGACCGACTTCAACGCCATCGCCACCTGCGACTGGACCAACGCCGGCGAC | |
| | TACATCCTGGCCAACACCTGCACCGAGAGGCTGAAGCTGTTCGCCGCCGAGA | |
| | CCCTGAAGGCCACCGAGGAGACCTTCAAGCTGAGCTACGGCATCGCCACCGT | |
| | GAGGGAGGTGCTGAGCGACAGGGAGCTGCACCTGAGCTGGGAGGTGGGCAA | |
| | GCCCAGGCCCCCCCTGAACAGGAACTACGTGTTCACCGGCTACAGGGTGACC | |
| | AAGAACAGCAAGGTGCAGATCGGCGAGTACACCTTCGAGAAGGGCGACTACG | |
| | GCGACGCCGTGGTGTACAGGGGCACCACCACCTACAAGCTGAACGTGGGCG | |
| | ACTACTTCGTGCTGACCAGCCACACCGTGATGCCCCTGAGCGCCCCCACCCT | |
| | GGTGCCCCAGGAGCACTACGTGAGGATCACCGGCCTGTACCCCACCCTGAAC | |
| | ATCAGCGACGAGTTCAGCAGCAACGTGGCCAACTACCAGAAGGTGGGCATGC | |
| | AGAAGTACAGCACCCTGCAGGGCCCCCCCGGCACCGGCAAGAGCCACTTCG | |
| | CCATCGGCCTGGCCCTGTACTACCCCAGCGCCAGGATCGTGTACACCGCCTG | |
| | CAGCCACGCCGCCGTGGACGCCCTGTGCGAGAAGGCCCTGAAGTACCTGCC | |
| | CATCGACAAGTGCAGCAGGATCATCCCCGCCAGGGCCAGGGTGGAGTGCTTC | |
| | GACAAGTTCAAGGTGAACAGCACCCTGGAGCAGTACGTGTTCTGCACCGTGA | |
| | ACGCCCTGCCCGAGACCACCGCCGACATCGTGGTGTTCGACGAGATCAGCAT | |
| | GGCCACCAACTACGACCTGAGCGTGGTGAACGCCAGGCTGAGGGCCAAGCA | |
| | CTACGTGTACATCGGCGACCCCGCCCAGCTGCCCGCCCCCAGGACCCTGCTG | |
| | ACCAAGGGCACCCTGGAGCCCGAGTACTTCAACAGCGTGTGCAGGCTGATGA | |
| | AGACCATCGGCCCCGACATGTTCCTGGGCACCTGCAGGAGGTGCCCCGCCG | |
| | AGATCGTGGACACCGTGAGCGCCCTGGTGTACGACAACAAGCTGAAGGCCCA | |
| | CAAGGACAAGAGCGCCCAGTGCTTCAAGATGTTCTACAAGGGCGTGATCACC | |
| | CACGACGTGAGCAGCGCCATCAACAGGCCCCAGATCGGCGTGGTGAGGGAG | |
| | TTCCTGACCAGGAACCCCGCCTGGAGGAAGGCCGTGTTCATCAGCCCCTACA | |
| | ACAGCCAGAACGCCGTGGCCAGCAAGATCCTGGGCCTGCCCACCCAGACCG | |
| | TGGACAGCAGCCAGGGCAGCGAGTACGACTACGTGATCTTCACCCAGACCAC | |
| | CGAGACCGCCCACAGCTGCAACGTGAACAGGTTCAACGTGGCCATCACCAGG | |
| | GCCAAGGTGGGCATCCTGTGCATCATGAGCGACAGGGACCTGTACGACAAGC | |
| | TGCAGTTCACCAGCCTGGAGATCCCCAGGAGGAACGTGGCCACCCTGCAGTG | |
| | ATGACTCGAGCTGGTACTGCATGCACGCAATGCTAGCTGCCCCTTTCCCGTCC | |
| | TGGGTACCCCGAGTCTCCCCCGACCTCGGGTCCCAGGTATGCTCCCACCTCC | |
| | ACCTGCCCCACTCACCACCTCTGCTAGTTCCAGACACCTCCCAAGCACGCAG | |
| | CAATGCAGCTCAAAACGCTTAGCCTAGCCACACCCCCACGGGAAACAGCAGT | |
| | GATTAACCTTTAGCAATAAACGAAAGTTTAACTAAGCTATACTAACCCCAGGGTT | |
| | GGTCAATTTCGTGCCAGCCACACCCTGGAGCTAGCAAAAAAAA | |
| Ad5-Pan- | TAGTTATTAATAGTAATCAATTACGGGGTCATTAGTTCATAGCCCATATATGGAGT | 69 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #6 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| NSP9-NS | AAATGGCCCGCCTGGCATTATGCCCAGTACATGACCTTATGGGACTTTCCTACT | |
| P10-NSP | TGGCAGTACATCTACGTATTAGTCATCGCTATTACCATGGTGATGCGGTTTTGGC | |
| 13 | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAACAA | |
| | CGAGCTGAGCCCCGTGGCCCTGAGGCAGATGAGCTGCGCCGCCGGCACCAC | |
| | CCAGACCGCCTGCACCGACGACAACGCCCTGGCCTACTACAACACCACCAAG | |
| | GGCGGCAGGTTCGTGCTGGCCCTGCTGAGCGACCTGCAGGACCTGAAGTGG | |
| | GCCAGGTTCCCCAAGAGCGACGGCACCGGCACCATCTACACCGAGCTGGAG | |
| | CCCCCCTGCAGGTTCGTGACCGACACCCCCAAGGGCCCCAAGGTGAAGTACC | |
| | TGTACTTCATCAAGGGCCTGAACAACCTGAACAGGGGCATGGTGCTGGGCAG | |
| | CCTGGCCGCCACCGTGAGGCTGCAGGGAAGCGGAGAGGGCAGGGGAAGTC | |
| | TTCTAACATGCGGGGACGTGGAGGAAAATCCCGGCCCCGCCGGCAACGCCAC | |
| | CGAGGTGCCCGCCAACAGCACCGTGCTGAGCTTCTGCGCCTTCGCCGTGGA | |
| | CGCCGCCAAGGCCTACAAGGACTACCTGGCCAGCGGCGGCCAGCCCATCAC | |
| | CAACTGCGTGAAGATGCTGTGCACCCACACCGGCACCGGCCAGGCCATCACC | |
| | GTGACCCCCGAGGCCAACATGGACCAGGAGAGCTTCGGCGGCGCCAGCTGC | |
| | TGCCTGTACTGCAGGTGCCACATCGACCACCCCAACCCCAAGGGCTTCTGCG | |
| | ACCTGAAGGGCAAGTACGTGCAGATCCCCACCACCTGCGCCAACGACCCCGT | |
| | GGGCTTCACCCTGAAGAACACCGTGTGCACCGTGTGCGGCATGTGGAAGGGC | |
| | TACGGCTGCAGCTGCGACCAGCTGAGGGAGCCCATGCTGCAGGGAAGCGGA | |
| | CAGTGTACTAATTATGCTCTCTTGAAATTGGCTGGAGATGTTGAGAGCAACCCA | |
| | GGTCCCGCCGTGGGCGCCTGCGTGCTGTGCAACAGCCAGACCAGCCTGAGG | |
| | TGCGGCGCCTGCATCAGGAGGCCCTTCCTGTGCTGCAAGTGCTGCTACGACC | |
| | ACGTGATCAGCACCAGCCACAAGCTGGTGCTGAGCGTGAACCCCTACGTGTG | |
| | CAACGCCCCCGGCTGCGACGTGACCGACGTGACCCAGCTGTACCTGGGCGG | |
| | CATGAGCTACTACTGCAAGAGCCACAAGCCCCCCATCAGCTTCCCCCTGTGCG | |
| | CCAACGGCCAGGTGTTCGGCCTGTACAAGAACACCTGCGTGGGCAGCGACAA | |
| | CGTGACCGACTTCAACGCCATCGCCACCTGCGACTGGACCAACGCCGGCGAC | |
| | TACATCCTGGCCAACACCTGCACCGAGAGGCTGAAGCTGTTCGCCGCCGAGA | |
| | CCCTGAAGGCCACCGAGGAGACCTTCAAGCTGAGCTACGGCATCGCCACCGT | |
| | GAGGGAGGTGCTGAGCGACAGGGAGCTGCACCTGAGCTGGGAGGTGGGCAA | |
| | GCCCAGGCCCCCCCTGAACAGGAACTACGTGTTCACCGGCTACAGGGTGACC | |
| | AAGAACAGCAAGGTGCAGATCGGCGAGTACACCTTCGAGAAGGGCGACTACG | |
| | GCGACGCCGTGGTGTACAGGGGCACCACCACCTACAAGCTGAACGTGGGCG | |
| | ACTACTTCGTGCTGACCAGCCACACCGTGATGCCCCTGAGCGCCCCCACCCT | |
| | GGTGCCCCAGGAGCACTACGTGAGGATCACCGGCCTGTACCCCACCCTGAAC | |
| | ATCAGCGACGAGTTCAGCAGCAACGTGGCCAACTACCAGAAGGTGGGCATGC | |
| | AGAAGTACAGCACCCTGCAGGGCCCCCCCGGCACCGGCAAGAGCCACTTCG | |
| | CCATCGGCCTGGCCCTGTACTACCCCAGCGCCAGGATCGTGTACACCGCCTG | |
| | CAGCCACGCCGCCGTGGACGCCCTGTGCGAGAAGGCCCTGAAGTACCTGCC | |
| | CATCGACAAGTGCAGCAGGATCATCCCCGCCAGGGCCAGGGTGGAGTGCTTC | |
| | GACAAGTTCAAGGTGAACAGCACCCTGGAGCAGTACGTGTTCTGCACCGTGA | |
| | ACGCCCTGCCCGAGACCACCGCCGACATCGTGGTGTTCGACGAGATCAGCAT | |
| | GGCCACCAACTACGACCTGAGCGTGGTGAACGCCAGGCTGAGGGCCAAGCA | |
| | CTACGTGTACATCGGCGACCCCGCCCAGCTGCCCGCCCCCAGGACCCTGCTG | |
| | ACCAAGGGCACCCTGGAGCCCGAGTACTTCAACAGCGTGTGCAGGCTGATGA | |
| | AGACCATCGGCCCCGACATGTTCCTGGGCACCTGCAGGAGGTGCCCCGCCG | |
| | AGATCGTGGACACCGTGAGCGCCCTGGTGTACGACAACAAGCTGAAGGCCCA | |
| | CAAGGACAAGAGCGCCCAGTGCTTCAAGATGTTCTACAAGGGCGTGATCACC | |
| | CACGACGTGAGCAGCGCCATCAACAGGCCCCAGATCGGCGTGGTGAGGGAG | |
| | TTCCTGACCAGGAACCCCGCCTGGAGGAAGGCCGTGTTCATCAGCCCCTACA | |
| | ACAGCCAGAACGCCGTGGCCAGCAAGATCCTGGGCCTGCCCACCCAGACCG | |
| | TGGACAGCAGCCAGGGCAGCGAGTACGACTACGTGATCTTCACCCAGACCAC | |
| | CGAGACCGCCCACAGCTGCAACGTGAACAGGTTCAACGTGGCCATCACCAGG | |
| | GCCAAGGTGGGCATCCTGTGCATCATGAGCGACAGGGACCTGTACGACAAGC | |
| | TGCAGTTCACCAGCCTGGAGATCCCCAGGAGGAACGTGGCCACCCTGCAGTG | |
| | ATGACTCGAGCTGGTACTGCATGCACGCAATGCTAGCTGCCCCTTTCCCGTCC | |
| | TGGGTACCCCGAGTCTCCCCCGACCTCGGGTCCCAGGTATGCTCCCACCTCC | |
| | ACCTGCCCCACTCACCACCTCTGCTAGTTCCAGACACCTCCCAAGCACGCAG | |
| | CAATGCAGCTCAAAACGCTTAGCCTAGCCACACCCCCACGGGAAACAGCAGT | |
| | GATTAACCTTTAGCAATAAACGAAAGTTTAACTAAGCTATACTAACCCCAGGGTT | |
| | GGTCAATTTCGTGCCAGCCACACCCTGGAGCTAGCAAAAAAAA | |
| Ad5-Pan- | TAGTTATTAATAGTAATCAATTACGGGGTCATTAGTTCATAGCCCATATATGGAGT | 70 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #7 | ACTTTCCATTGACGTCAATGGGTGGAGTATTTACGGTAAACTGCCCACTTGGCA | |
| Spike-NS | GTACATCAAGTGTATCATATGCCAAGTACGCCCCCTATTGACGTCAATGACGGT | |
| P14-NSP | AAATGGCCCGCCTGGCATTATGCCCAGTACATGACCTTATGGGACTTTCCTACT | |
| 15 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCGA | |
| | GAACGTGACCGGCCTGTTCAAGGACTGCAGCAAGGTGATCACCGGCCTGCAC | |
| | CCCACCCAGGCCCCCACCCACCTGAGCGTGGACACCAAGTTCAAGACCGAG | |
| | GGCCTGTGCGTGGACATCCCCGGCATCCCCAAGGACATGACCTACAGGAGGC | |
| | TGATCAGCATGATGGGCTTCAAGATGAACTACCAGGTGAACGGCTACCCCAAC | |
| | ATGTTCATCACCAGGGAGGAGGCCATCAGGCACGTGAGGGCCTGGATCGGCT | |
| | TCGACGTGGAGGGCTGCCACGCCACCAGGGAGGCCGTGGGCACCAACCTGC | |
| | CCCTGCAGCTGGGCTTCAGCACCGGCGTGAACCTGGTGGCCGTGCCCACCG | |
| | GCTACGTGGACACCCCCAACAACACCGACTTCAGCAGGGTGAGCGCCAAGCC | |
| | CCCCCCCGGCGACCAGTTCAAGCACCTGATCCCCCTGATGTACAAGGGCCTG | |
| | CCCTGGAACGTGGTGAGGATCAAGATCGTGCAGATGCTGAGCGACACCCTGA | |
| | AGAACCTGAGCGACAGGGTGGTGTTCGTGCTGTGGGCCCACGGCTTCGAGC | |
| | TGACCAGCATGAAGTACTTCGTGAAGATCGGCCCCGAGAGGACCTGCTGCCT | |
| | GTGCGACAGGAGGGCCACCTGCTTCAGCACCGCCAGCGACACCTACGCCTG | |
| | CTGGCACCACAGCATCGGCTTCGACTACGTGTACAACCCCTTCATGATCGACG | |
| | TGCAGCAGTGGGGCTTCACCGGCAACCTGCAGAGCAACCACGACCTGTACTG | |
| | CCAGGTGCACGGCAACGCCCACGTGGCCAGCTGCGACGCCATCATGACCAG | |
| | GTGCCTGGCCGTGCACGAGTGCTTCGTGAAGAGGGTGGACTGGACCATCGA | |
| | GTACCCCATCATCGGCGACGAGCTGAAGATCAACGCCGCCTGCAGGAAGGTG | |
| | CAGCACATGGTGGTGAAGGCCGCCCTGCTGGCCGACAAGTTCCCCGTGCTGC | |
| | ACGACATCGGCAACCCCAAGGCCATCAAGTGCGTGCCCCAGGCCGACGTGGA | |
| | GTGGAAGTTCTACGACGCCCAGCCCTGCAGCGACAAGGCCTACAAGATCGAG | |
| | GAGCTGTTCTACAGCTACGCCACCCACAGCGACAAGTTCACCGACGGCGTGT | |
| | GCCTGTTCTGGAACTGCAACGTGGACAGGTACCCCGCCAACAGCATCGTGTG | |
| | CAGGTTCGACACCAGGGTGCTGAGCAACCTGAACCTGCCCGGCTGCGACGG | |
| | CGGCAGCCTGTACGTGAACAAGCACGCCTTCCACACCCCCGCCTTCGACAAG | |
| | AGCGCCTTCGTGAACCTGAAGCAGCTGCCCTTCTTCTACTACAGCGACAGCCC | |
| | CTGCGAGAGCCACGGCAAGCAGGTGGTGAGCGACATCGACTACGTGCCCCT | |
| | GAAGAGCGCCACCTGCATCACCAGGTGCAACCTGGGCGGCGCCGTGTGCAG | |
| | GCACCACGCCAACGAGTACAGGCTGTACCTGGACGCCTACAACATGATGATCA | |
| | GCGCCGGCTTCAGCCTGTGGGTGTACAAGCAGTTCGACACCTACAACCTGTG | |
| | GAACACCTTCACCAGGCTGCAGGGAAGCGGAGAGGGCAGGGGAAGTCTTCTA | |
| | ACATGCGGGGACGTGGAGGAAAATCCCGGCCCCAGCCTGGAGAACGTGGCC | |
| | TTCAACGTGGTGAACAAGGGCCACTTCGACGGCCAGCAGGGCGAGGTGCCC | |
| | GTGAGCATCATCAACAACACCGTGTACACCAAGGTGGACGGCGTGGACGTGG | |
| | AGCTGTTCGAGAACAAGACCACCCTGCCCGTGAACGTGGCCTTCGAGCTGTG | |
| | GGCCAAGAGGAACATCAAGCCCGTGCCCGAGGTGAAGATCCTGAACAACCTG | |
| | GGCGTGGACATCGCCGCCAACACCGTGATCTGGGACTACAAGAGGGACGCCC | |
| | CCGCCCACATCAGCACCATCGGCGTGTGCAGCATGACCGACATCGCCAAGAA | |
| | GCCCACCGAGACCATCTGCGCCCCCCTGACCGTGTTCTTCGACGGCAGGGTG | |
| | GACGGCCAGGTGGACCTGTTCAGGAACGCCAGGAACGGCGTGCTGATCACC | |
| | GAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTAC | |
| | TACAAGAAGGTGGACGGCGTGGTGCAGCAGCTGCCCGAGACCTACTTCACCC | |
| | AGAGCAGGAACCTGCAGGAGTTCAAGCCCAGGAGCCAGATGGAGATCGACTT | |
| | CCTGGAGCTGGCCATGGACGAGTTCATCGAGAGGTACAAGCTGGAGGGCTAC | |
| | GCCTTCGAGCACATCGTGTACGGCGACTTCAGCCACAGCCAGCTGGGCGGCC | |
| | TGCACCTGCTGATCGGCCTGGCCAAGAGGTTCAAGGAGAGCCCCTTCGAGCT | |
| | GGAGGACTTCATCCCCATGGACAGCACCGTGAAGAACTACTTCATCACCGACG | |
| | CCCAGACCGGCAGCAGCAAGTGCGTGTGCAGCGTGATCGACCTGCTGCTGG | |
| | ACGACTTCGTGGAGATCATCAAGAGCCAGGACCTGAGCGTGGTGAGCAAGGT | |
| | GGTGAAGGTGACCATCGACTACACCGAGATCAGCTTCATGCTGTGGTGCAAGG | |
| | ACGGCCACGTGGAGACCTTCTACCCCAAGCTGCAGTGATGACTCGAGCTGGT | |
| | ACTGCATGCACGCAATGCTAGCTGCCCCTTTCCCGTCCTGGGTACCCCGAGTC | |
| | TCCCCCGACCTCGGGTCCCAGGTATGCTCCCACCTCCACCTGCCCCACTCAC | |
| | CACCTCTGCTAGTTCCAGACACCTCCCAAGCACGCAGCAATGCAGCTCAAAAC | |
| | GCTTAGCCTAGCCACACCCCCACGGGAAACAGCAGTGATTAACCTTTAGCAATA | |
| | AACGAAAGTTTAACTAAGCTATACTAACCCCAGGGTTGGTCAATTTCGTGCCAG | |
| | CCACACCCTGGAGCTAGCAAAAAAAA | |
| Ad5-Pan- | TAGTTATTAATAGTAATCAATTACGGGGTCATTAGTTCATAGCCCATATATGGAGT | 71 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #8 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| NSP14-N | AAATGGCCCGCCTGGCATTATGCCCAGTACATGACCTTATGGGACTTTCCTACT | |
| SP15 | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCGA | |
| | GAACGTGACCGGCCTGTTCAAGGACTGCAGCAAGGTGATCACCGGCCTGCAC | |
| | CCCACCCAGGCCCCCACCCACCTGAGCGTGGACACCAAGTTCAAGACCGAG | |
| | GGCCTGTGCGTGGACATCCCCGGCATCCCCAAGGACATGACCTACAGGAGGC | |
| | TGATCAGCATGATGGGCTTCAAGATGAACTACCAGGTGAACGGCTACCCCAAC | |
| | ATGTTCATCACCAGGGAGGAGGCCATCAGGCACGTGAGGGCCTGGATCGGCT | |
| | TCGACGTGGAGGGCTGCCACGCCACCAGGGAGGCCGTGGGCACCAACCTGC | |
| | CCCTGCAGCTGGGCTTCAGCACCGGCGTGAACCTGGTGGCCGTGCCCACCG | |
| | GCTACGTGGACACCCCCAACAACACCGACTTCAGCAGGGTGAGCGCCAAGCC | |
| | CCCCCCCGGCGACCAGTTCAAGCACCTGATCCCCCTGATGTACAAGGGCCTG | |
| | CCCTGGAACGTGGTGAGGATCAAGATCGTGCAGATGCTGAGCGACACCCTGA | |
| | AGAACCTGAGCGACAGGGTGGTGTTCGTGCTGTGGGCCCACGGCTTCGAGC | |
| | TGACCAGCATGAAGTACTTCGTGAAGATCGGCCCCGAGAGGACCTGCTGCCT | |
| | GTGCGACAGGAGGGCCACCTGCTTCAGCACCGCCAGCGACACCTACGCCTG | |
| | CTGGCACCACAGCATCGGCTTCGACTACGTGTACAACCCCTTCATGATCGACG | |
| | TGCAGCAGTGGGGCTTCACCGGCAACCTGCAGAGCAACCACGACCTGTACTG | |
| | CCAGGTGCACGGCAACGCCCACGTGGCCAGCTGCGACGCCATCATGACCAG | |
| | GTGCCTGGCCGTGCACGAGTGCTTCGTGAAGAGGGTGGACTGGACCATCGA | |
| | GTACCCCATCATCGGCGACGAGCTGAAGATCAACGCCGCCTGCAGGAAGGTG | |
| | CAGCACATGGTGGTGAAGGCCGCCCTGCTGGCCGACAAGTTCCCCGTGCTGC | |
| | ACGACATCGGCAACCCCAAGGCCATCAAGTGCGTGCCCCAGGCCGACGTGGA | |
| | GTGGAAGTTCTACGACGCCCAGCCCTGCAGCGACAAGGCCTACAAGATCGAG | |
| | GAGCTGTTCTACAGCTACGCCACCCACAGCGACAAGTTCACCGACGGCGTGT | |
| | GCCTGTTCTGGAACTGCAACGTGGACAGGTACCCCGCCAACAGCATCGTGTG | |
| | CAGGTTCGACACCAGGGTGCTGAGCAACCTGAACCTGCCCGGCTGCGACGG | |
| | CGGCAGCCTGTACGTGAACAAGCACGCCTTCCACACCCCCGCCTTCGACAAG | |
| | AGCGCCTTCGTGAACCTGAAGCAGCTGCCCTTCTTCTACTACAGCGACAGCCC | |
| | CTGCGAGAGCCACGGCAAGCAGGTGGTGAGCGACATCGACTACGTGCCCCT | |
| | GAAGAGCGCCACCTGCATCACCAGGTGCAACCTGGGCGGCGCCGTGTGCAG | |
| | GCACCACGCCAACGAGTACAGGCTGTACCTGGACGCCTACAACATGATGATCA | |
| | GCGCCGGCTTCAGCCTGTGGGTGTACAAGCAGTTCGACACCTACAACCTGTG | |
| | GAACACCTTCACCAGGCTGCAGGGAAGCGGAGAGGGCAGGGGAAGTCTTCTA | |
| | ACATGCGGGGACGTGGAGGAAAATCCCGGCCCCAGCCTGGAGAACGTGGCC | |
| | TTCAACGTGGTGAACAAGGGCCACTTCGACGGCCAGCAGGGCGAGGTGCCC | |
| | GTGAGCATCATCAACAACACCGTGTACACCAAGGTGGACGGCGTGGACGTGG | |
| | AGCTGTTCGAGAACAAGACCACCCTGCCCGTGAACGTGGCCTTCGAGCTGTG | |
| | GGCCAAGAGGAACATCAAGCCCGTGCCCGAGGTGAAGATCCTGAACAACCTG | |
| | GGCGTGGACATCGCCGCCAACACCGTGATCTGGGACTACAAGAGGGACGCCC | |
| | CCGCCCACATCAGCACCATCGGCGTGTGCAGCATGACCGACATCGCCAAGAA | |
| | GCCCACCGAGACCATCTGCGCCCCCCTGACCGTGTTCTTCGACGGCAGGGTG | |
| | GACGGCCAGGTGGACCTGTTCAGGAACGCCAGGAACGGCGTGCTGATCACC | |
| | GAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTAC | |
| | TACAAGAAGGTGGACGGCGTGGTGCAGCAGCTGCCCGAGACCTACTTCACCC | |
| | AGAGCAGGAACCTGCAGGAGTTCAAGCCCAGGAGCCAGATGGAGATCGACTT | |
| | CCTGGAGCTGGCCATGGACGAGTTCATCGAGAGGTACAAGCTGGAGGGCTAC | |
| | GCCTTCGAGCACATCGTGTACGGCGACTTCAGCCACAGCCAGCTGGGCGGCC | |
| | TGCACCTGCTGATCGGCCTGGCCAAGAGGTTCAAGGAGAGCCCCTTCGAGCT | |
| | GGAGGACTTCATCCCCATGGACAGCACCGTGAAGAACTACTTCATCACCGACG | |
| | CCCAGACCGGCAGCAGCAAGTGCGTGTGCAGCGTGATCGACCTGCTGCTGG | |
| | ACGACTTCGTGGAGATCATCAAGAGCCAGGACCTGAGCGTGGTGAGCAAGGT | |
| | GGTGAAGGTGACCATCGACTACACCGAGATCAGCTTCATGCTGTGGTGCAAGG | |
| | ACGGCCACGTGGAGACCTTCTACCCCAAGCTGCAGTGATGACTCGAGCTGGT | |
| | ACTGCATGCACGCAATGCTAGCTGCCCCTTTCCCGTCCTGGGTACCCCGAGTC | |
| | TCCCCCGACCTCGGGTCCCAGGTATGCTCCCACCTCCACCTGCCCCACTCAC | |
| | CACCTCTGCTAGTTCCAGACACCTCCCAAGCACGCAGCAATGCAGCTCAAAAC | |
| | GCTTAGCCTAGCCACACCCCCACGGGAAACAGCAGTGATTAACCTTTAGCAATA | |
| | AACGAAAGTTTAACTAAGCTATACTAACCCCAGGGTTGGTCAATTTCGTGCCAG | |
| | CCACACCCTGGAGCTAGCAAAAAAAA | |
| Ad5-Pan- | TAGTTATTAATAGTAATCAATTACGGGGTCATTAGTTCATAGCCCATATATGGAGT | 72 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #9 | ACTTTCCATTGACGTCAATGGGTGGAGTATTTACGGTAAACTGCCCACTTGGCA | |
| Spike-NS | GTACATCAAGTGTATCATATGCCAAGTACGCCCCCTATTGACGTCAATGACGGT | |
| P16 | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCAG | |
| | CCAGGCCTGGCAGCCCGGCGTGGCCATGCCCAACCTGTACAAGATGCAGAG | |
| | GATGCTGCTGGAGAAGTGCGACCTGCAGAACTACGGCGACAGCGCCACCCTG | |
| | CCCAAGGGCATCATGATGAACGTGGCCAAGTACACCCAGCTGTGCCAGTACCT | |
| | GAACACCCTGACCCTGGCCGTGCCCTACAACATGAGGGTGATCCACTTCGGC | |
| | GCCGGCAGCGACAAGGGCGTGGCCCCCGGCACCGCCGTGCTGAGGCAGTG | |
| | GCTGCCCACCGGCACCCTGCTGGTGGACAGCGACCTGAACGACTTCGTGAG | |
| | CGACGCCGACAGCACCCTGATCGGCGACTGCGCCACCGTGCACACCGCCAA | |
| | CAAGTGGGACCTGATCATCAGCGACATGTACGACCCCAAGACCAAGAACGTGA | |
| | CCAAGGAGAACGACAGCAAGGAGGGCTTCTTCACCTACATCTGCGGCTTCATC | |
| | CAGCAGAAGCTGGCCCTGGGCGGCAGCGTGGCCATCAAGATCACCGAGCAC | |
| | AGCTGGAACGCCGACCTGTACAAGCTGATGGGCCACTTCGCCTGGTGGACCG | |
| | CCTTCGTGACCAACGTGAACGCCAGCAGCAGCGAGGCCTTCCTGATCGGCTG | |
| | CAACTACCTGGGCAAGCCCAGGGAGCAGATCGACGGCTACGTGATGCACGCC | |
| | AACTACATCTTCTGGAGGAACACCAACCCCATCCAGCTGAGCAGCTACAGCCT | |
| | GTTCGACATGAGCAAGTTCCCCCTGAAGCTGAGGGGCACCGCCGTGATGAGC | |
| | CTGAAGGAGGGCCAGATCAACGACATGATCCTGAGCCTGCTGAGCAAGGGCA | |
| | GGCTGATCATCAGGGAGAACAACAGGGTGGTGATCAGCAGCGACGTGCTGGT | |
| | GAACAACTGATGACTCGAGCTGGTACTGCATGCACGCAATGCTAGCTGCCCCT | |
| | TTCCCGTCCTGGGTACCCCGAGTCTCCCCCGACCTCGGGTCCCAGGTATGCT | |
| | CCCACCTCCACCTGCCCCACTCACCACCTCTGCTAGTTCCAGACACCTCCCAA | |
| | GCACGCAGCAATGCAGCTCAAAACGCTTAGCCTAGCCACACCCCCACGGGAA | |
| | ACAGCAGTGATTAACCTTTAGCAATAAACGAAAGTTTAACTAAGCTATACTAACC | |
| | CCAGGGTTGGTCAATTTCGTGCCAGCCACACCCTGGAGCTAGCAAAAAAAA | |
| Ad5-Pan- | TAGTTATTAATAGTAATCAATTACGGGGTCATTAGTTCATAGCCCATATATGGAGT | 73 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #10 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| NSP16 | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCAG | |
| | CCAGGCCTGGCAGCCCGGCGTGGCCATGCCCAACCTGTACAAGATGCAGAG | |
| | GATGCTGCTGGAGAAGTGCGACCTGCAGAACTACGGCGACAGCGCCACCCTG | |
| | CCCAAGGGCATCATGATGAACGTGGCCAAGTACACCCAGCTGTGCCAGTACCT | |
| | GAACACCCTGACCCTGGCCGTGCCCTACAACATGAGGGTGATCCACTTCGGC | |
| | GCCGGCAGCGACAAGGGCGTGGCCCCCGGCACCGCCGTGCTGAGGCAGTG | |
| | GCTGCCCACCGGCACCCTGCTGGTGGACAGCGACCTGAACGACTTCGTGAG | |
| | CGACGCCGACAGCACCCTGATCGGCGACTGCGCCACCGTGCACACCGCCAA | |
| | CAAGTGGGACCTGATCATCAGCGACATGTACGACCCCAAGACCAAGAACGTGA | |
| | CCAAGGAGAACGACAGCAAGGAGGGCTTCTTCACCTACATCTGCGGCTTCATC | |
| | CAGCAGAAGCTGGCCCTGGGCGGCAGCGTGGCCATCAAGATCACCGAGCAC | |
| | AGCTGGAACGCCGACCTGTACAAGCTGATGGGCCACTTCGCCTGGTGGACCG | |
| | CCTTCGTGACCAACGTGAACGCCAGCAGCAGCGAGGCCTTCCTGATCGGCTG | |
| | CAACTACCTGGGCAAGCCCAGGGAGCAGATCGACGGCTACGTGATGCACGCC | |
| | AACTACATCTTCTGGAGGAACACCAACCCCATCCAGCTGAGCAGCTACAGCCT | |
| | GTTCGACATGAGCAAGTTCCCCCTGAAGCTGAGGGGCACCGCCGTGATGAGC | |
| | CTGAAGGAGGGCCAGATCAACGACATGATCCTGAGCCTGCTGAGCAAGGGCA | |
| | GGCTGATCATCAGGGAGAACAACAGGGTGGTGATCAGCAGCGACGTGCTGGT | |
| | GAACAACTGATGACTCGAGCTGGTACTGCATGCACGCAATGCTAGCTGCCCCT | |
| | TTCCCGTCCTGGGTACCCCGAGTCTCCCCCGACCTCGGGTCCCAGGTATGCT | |
| | CCCACCTCCACCTGCCCCACTCACCACCTCTGCTAGTTCCAGACACCTCCCAA | |
| | GCACGCAGCAATGCAGCTCAAAACGCTTAGCCTAGCCACACCCCCACGGGAA | |
| | ACAGCAGTGATTAACCTTTAGCAATAAACGAAAGTTTAACTAAGCTATACTAACC | |
| | CCAGGGTTGGTCAATTTCGTGCCAGCCACACCCTGGAGCTAGCAAAAAAAA | |
| Ad5-Pan- | TAGTTATTAATAGTAATCAATTACGGGGTCATTAGTTCATAGCCCATATATGGAGT | 74 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #11 | ACTTTCCATTGACGTCAATGGGTGGAGTATTTACGGTAAACTGCCCACTTGGCA | |
| Spike-OR | GTACATCAAGTGTATCATATGCCAAGTACGCCCCCTATTGACGTCAATGACGGT | |
| F3a | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | CCTGTTCATGAGGATCTTCACCATCGGCACCGTGACCCTGAAGCAGGGCGAG | |
| | ATCAAGGACGCCACCCCCAGCGACTTCGTGAGGGCCACCGCCACCATCCCCA | |
| | TCCAGGCCAGCCTGCCCTTCGGCTGGCTGATCGTGGGCGTGGCCCTGCTGG | |
| | CCGTGTTCCAGAGCGCCAGCAAGATCATCACCCTGAAGAAGAGGTGGCAGCT | |
| | GGCCCTGAGCAAGGGCGTGCACTTCGTGTGCAACCTGCTGCTGCTGTTCGTG | |
| | ACCGTGTACAGCCACCTGCTGCTGGTGGCCGCCGGCCTGGAGGCCCCCTTC | |
| | CTGTACCTGTACGCCCTGGTGTACTTCCTGCAGAGCATCAACTTCGTGAGGAT | |
| | CATCATGAGGCTGTGGCTGTGCTGGAAGTGCAGGAGCAAGAACCCCCTGCTG | |
| | TACGACGCCAACTACTTCCTGTGCTGGCACACCAACTGCTACGACTACTGCAT | |
| | CCCCTACAACAGCGTGACCAGCAGCATCGTGATCACCAGCGGCGACGGCACC | |
| | ACCAGCCCCATCAGCGAGCACGACTACCAGATCGGCGGCTACACCGAGAAGT | |
| | GGGAGAGCGGCGTGAAGGACTGCGTGGTGCTGCACAGCTACTTCACCAGCG | |
| | ACTACTACCAGCTGTACAGCACCCAGCTGAGCACCGACACCGGCGTGGAGCA | |
| | CGTGACCTTCTTCATCTACAACAAGATCGTGGACGAGCCCGAGGAGCACGTG | |
| | CAGATCCACACCATCGACGGCAGCAGCGGCGTGGTGAACCCCGTGATGGAGC | |
| | CCATCTACGACGAGCCCACCACCACCACCAGCGTGCCCCTGTGATGACTCGA | |
| | GCTGGTACTGCATGCACGCAATGCTAGCTGCCCCTTTCCCGTCCTGGGTACCC | |
| | CGAGTCTCCCCCGACCTCGGGTCCCAGGTATGCTCCCACCTCCACCTGCCCC | |
| | ACTCACCACCTCTGCTAGTTCCAGACACCTCCCAAGCACGCAGCAATGCAGCT | |
| | CAAAACGCTTAGCCTAGCCACACCCCCACGGGAAACAGCAGTGATTAACCTTT | |
| | AGCAATAAACGAAAGTTTAACTAAGCTATACTAACCCCAGGGTTGGTCAATTTCG | |
| | TGCCAGCCACACCCTGGAGCTAGCAAAAAAAA | |
| Ad5-Pan- | TAGTTATTAATAGTAATCAATTACGGGGTCATTAGTTCATAGCCCATATATGGAGT | 75 |
| CoV | TCCGCGTTACATAACTTACGGTAAATGGCCCGCCTGGCTGACCGCCCAACGAC | |
| Vaccine | CCCCGCCCATTGACGTCAATAATGACGTATGTTCCCATAGTAACGCCAATAGGG | |
| #12 | ACTTTCCATTGACGTCAATGGGTGGAGTATTTACGGTAAACTGCCCACTTGGCA | |
| HexaPro- | GTACATCAAGTGTATCATATGCCAAGTACGCCCCCTATTGACGTCAATGACGGT | |
| ORF3a | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | CCTGTTCATGAGGATCTTCACCATCGGCACCGTGACCCTGAAGCAGGGCGAG | |
| | ATCAAGGACGCCACCCCCAGCGACTTCGTGAGGGCCACCGCCACCATCCCCA | |
| | TCCAGGCCAGCCTGCCCTTCGGCTGGCTGATCGTGGGCGTGGCCCTGCTGG | |
| | CCGTGTTCCAGAGCGCCAGCAAGATCATCACCCTGAAGAAGAGGTGGCAGCT | |
| | GGCCCTGAGCAAGGGCGTGCACTTCGTGTGCAACCTGCTGCTGCTGTTCGTG | |
| | ACCGTGTACAGCCACCTGCTGCTGGTGGCCGCCGGCCTGGAGGCCCCCTTC | |
| | CTGTACCTGTACGCCCTGGTGTACTTCCTGCAGAGCATCAACTTCGTGAGGAT | |
| | CATCATGAGGCTGTGGCTGTGCTGGAAGTGCAGGAGCAAGAACCCCCTGCTG | |
| | TACGACGCCAACTACTTCCTGTGCTGGCACACCAACTGCTACGACTACTGCAT | |
| | CCCCTACAACAGCGTGACCAGCAGCATCGTGATCACCAGCGGCGACGGCACC | |
| | ACCAGCCCCATCAGCGAGCACGACTACCAGATCGGCGGCTACACCGAGAAGT | |
| | GGGAGAGCGGCGTGAAGGACTGCGTGGTGCTGCACAGCTACTTCACCAGCG | |
| | ACTACTACCAGCTGTACAGCACCCAGCTGAGCACCGACACCGGCGTGGAGCA | |
| | CGTGACCTTCTTCATCTACAACAAGATCGTGGACGAGCCCGAGGAGCACGTG | |
| | CAGATCCACACCATCGACGGCAGCAGCGGCGTGGTGAACCCCGTGATGGAGC | |
| | CCATCTACGACGAGCCCACCACCACCACCAGCGTGCCCCTGTGATGACTCGA | |
| | GCTGGTACTGCATGCACGCAATGCTAGCTGCCCCTTTCCCGTCCTGGGTACCC | |
| | CGAGTCTCCCCCGACCTCGGGTCCCAGGTATGCTCCCACCTCCACCTGCCCC | |
| | ACTCACCACCTCTGCTAGTTCCAGACACCTCCCAAGCACGCAGCAATGCAGCT | |
| | CAAAACGCTTAGCCTAGCCACACCCCCACGGGAAACAGCAGTGATTAACCTTT | |
| | AGCAATAAACGAAAGTTTAACTAAGCTATACTAACCCCAGGGTTGGTCAATTTCG | |
| | TGCCAGCCACACCCTGGAGCTAGCAAAAAAAA | |
| Pan-CoV | ATGTTCGTGTTCCTGGTGCTGCTGCCCCTGGTGAGCAGCCAGTGCGTGAACC | 76 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #13 | GACCTGTTCCTGCCCTTCTTCAGCAACGTGACCTGGTTCCACGCCATCCACGT | |
| Spike-NS | GAGCGGCACCAACGGCACCAAGAGGTTCGACAACCCCGTGCTGCCCTTCAAC | |
| P1-NSP2 | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | GAGCCTGGTGCCCGGCTTCAACGAGAAGACCCACGTGCAGCTGAGCCTGCC | |
| | CGTGCTGCAGGTGAGGGACGTGCTGGTGAGGGGCTTCGGCGACAGCGTGGA | |
| | GGAGGTGCTGAGCGAGGCCAGGCAGCACCTGAAGGACGGCACCTGCGGCCT | |
| | GGTGGAGGTGGAGAAGGGCGTGCTGCCCCAGCTGGAGCAGCCCTACGTGTT | |
| | CATCAAGAGGAGCGACGCCAGGACCGCCCCCCACGGCCACGTGATGGTGGA | |
| | GCTGGTGGCCGAGCTGGAGGGCATCCAGTACGGCAGGAGCGGCGAGACCCT | |
| | GGGCGTGCTGGTGCCCCACGTGGGCGAGATCCCCGTGGCCTACAGGAAGGT | |
| | GCTGCTGAGGAAGAACGGCAACAAGGGCGCCGGCGGCCACAGCTACGGCGC | |
| | CGACCTGAAGAGCTTCGACCTGGGCGACGAGCTGGGCACCGACCCCTACGA | |
| | GGACTTCCAGGAGAACTGGAACACCAAGCACAGCAGCGGCGTGACCAGGGA | |
| | GCTGATGAGGGAGCTGAACGGCGGCGGAAGCGGAGAGGGCAGGGGAAGTCT | |
| | TCTAACATGCGGGGACGTGGAGGAAAATCCCGGCCCCGCCTACACCAGGTAC | |
| | GTGGACAACAACTTCTGCGGCCCCGACGGCTACCCCCTGGAGTGCATCAAGG | |
| | ACCTGCTGGCCAGGGCCGGCAAGGCCAGCTGCACCCTGAGCGAGCAGCTGG | |
| | ACTTCATCGACACCAAGAGGGGCGTGTACTGCTGCAGGGAGCACGAGCACGA | |
| | GATCGCCTGGTACACCGAGAGGAGCGAGAAGAGCTACGAGCTGCAGACCCCC | |
| | TTCGAGATCAAGCTGGCCAAGAAGTTCGACACCTTCAACGGCGAGTGCCCCA | |
| | ACTTCGTGTTCCCCCTGAACAGCATCATCAAGACCATCCAGCCCAGGGTGGAG | |
| | AAGAAGAAGCTGGACGGCTTCATGGGCAGGATCAGGAGCGTGTACCCCGTGG | |
| | CCAGCCCCAACGAGTGCAACCAGATGTGCCTGAGCACCCTGATGAAGTGCGA | |
| | CCACTGCGGCGAGACCAGCTGGCAGACCGGCGACTTCGTGAAGGCCACCTG | |
| | CGAGTTCTGCGGCACCGAGAACCTGACCAAGGAGGGCGCCACCACCTGCGG | |
| | CTACCTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAGCGGC | |
| | CTGAAGACCATCCTGAGGAAGGGCGGCAGGACCATCGCCTTCGGCGGCTGC | |
| | GTGTTCAGCTACGTGGGCTGCCACAACAAGTGCGCCTACTGGGTGCCCAGGG | |
| | CCAGCGCCAACATCGGCTGCAACCACACCGGCGTGGTGGGCGAGGGCAGCG | |
| | AGGGCCTGAACGACAACCTGCTGGAGATCCTGCAGAAGGAGAAGGTGAACAT | |
| | CAACATCGTGGGCGACTTCAAGCTGAACGAGGAGATCGCCATCATCCTGGCCA | |
| | GCTTCAGCGCCAGCACCAGCGCCTTCGTGGAGACCGTGAAGGGCCTGGACTA | |
| | CAAGGCCTTCAAGCAGATCGTGGAGAGCTGCGGCAACTTCAAGGTGACCAAG | |
| | GGCAAGGCCAAGAAGGGCGCCTGGAACATCGGCGAGCAGAAGAGCATCCTG | |
| | AGCCCCCTGTACGCCTTCGCCAGCGAGGCCGCCAGGGTGGTGAGGAGCATC | |
| | TTCAGCAGGACCCTGGAGACCGCCCAGAACAGCGTGAGGGTGCTGCAGAAG | |
| | GCCGCCATCACCATCCTGGACGGCATCAGCCAGTACAGCCTGAGGCTGATCG | |
| | ACGCCATGATGTTCACCAGCGACCTGGCCACCAACAACCTGGTGGTGATGGC | |
| | CTACATCACCGGCGGCGTGGTGCAGCTGACCAGCCAGTGGCTGACCAACATC | |
| | TTCGGCACCGTGTACGAGAAGCTGAAGCCCGTGCTGGACTGGCTGGAGGAGA | |
| | AGTTCAAGGAGGGCGTGGAGTTCCTGAGGGACGGCTGGGAGATCGTGAAGTT | |
| | CATCAGCACCTGCGCCTGCGAGATCGTGGGCGGCCAGATCGTGACCTGCGCC | |
| | AAGGAGATCAAGGAGAGCGTGCAGACCTTCTTCAAGCTGGTGAACAAGTTCCT | |
| | GGCCCTGTGCGCCGACAGCATCATCATCGGCGGCGCCAAGCTGAAGGCCCT | |
| | GAACCTGGGCGAGACCTTCGTGACCCACAGCAAGGGCCTGTACAGGAAGTGC | |
| | GTGAAGAGCAGGGAGGAGACCGGCCTGCTGATGCCCCTGAAGGCCCCCAAG | |
| | GAGATCATCTTCCTGGAGGGCGAGACCCTGCCCACCGAGGTGCTGACCGAGG | |
| | AGGTGGTGCTGAAGACCGGCGACCTGCAGCCCCTGGAGCAGCCCACCAGCG | |
| | AGGCCGTGGAGGCCCCCCTGGTGGGCACCCCCGTGTGCATCAACGGCCTGA | |
| | TGCTGCTGGAGATCAAGGACACCGAGAAGTACTGCGCCCTGGCCCCCAACAT | |
| | GATGGTGACCAACAACACCTTCACCCTGAAGGGCGGCTGATGA | |
| Pan-CoV | ATGTTCGTGTTCCTGGTGCTGCTGCCCCTGGTGAGCAGCCAGTGCGTGAACC | 77 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #14 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| NSP1-NS | GACGGCGTGTACTTCGCCAGCACCGAGAAGAGCAACATCATCAGGGGCTGGA | |
| P2 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | GAGCCTGGTGCCCGGCTTCAACGAGAAGACCCACGTGCAGCTGAGCCTGCC | |
| | CGTGCTGCAGGTGAGGGACGTGCTGGTGAGGGGCTTCGGCGACAGCGTGGA | |
| | GGAGGTGCTGAGCGAGGCCAGGCAGCACCTGAAGGACGGCACCTGCGGCCT | |
| | GGTGGAGGTGGAGAAGGGCGTGCTGCCCCAGCTGGAGCAGCCCTACGTGTT | |
| | CATCAAGAGGAGCGACGCCAGGACCGCCCCCCACGGCCACGTGATGGTGGA | |
| | GCTGGTGGCCGAGCTGGAGGGCATCCAGTACGGCAGGAGCGGCGAGACCCT | |
| | GGGCGTGCTGGTGCCCCACGTGGGCGAGATCCCCGTGGCCTACAGGAAGGT | |
| | GCTGCTGAGGAAGAACGGCAACAAGGGCGCCGGCGGCCACAGCTACGGCGC | |
| | CGACCTGAAGAGCTTCGACCTGGGCGACGAGCTGGGCACCGACCCCTACGA | |
| | GGACTTCCAGGAGAACTGGAACACCAAGCACAGCAGCGGCGTGACCAGGGA | |
| | GCTGATGAGGGAGCTGAACGGCGGCGGAAGCGGAGAGGGCAGGGGAAGTCT | |
| | TCTAACATGCGGGGACGTGGAGGAAAATCCCGGCCCCGCCTACACCAGGTAC | |
| | GTGGACAACAACTTCTGCGGCCCCGACGGCTACCCCCTGGAGTGCATCAAGG | |
| | ACCTGCTGGCCAGGGCCGGCAAGGCCAGCTGCACCCTGAGCGAGCAGCTGG | |
| | ACTTCATCGACACCAAGAGGGGCGTGTACTGCTGCAGGGAGCACGAGCACGA | |
| | GATCGCCTGGTACACCGAGAGGAGCGAGAAGAGCTACGAGCTGCAGACCCCC | |
| | TTCGAGATCAAGCTGGCCAAGAAGTTCGACACCTTCAACGGCGAGTGCCCCA | |
| | ACTTCGTGTTCCCCCTGAACAGCATCATCAAGACCATCCAGCCCAGGGTGGAG | |
| | AAGAAGAAGCTGGACGGCTTCATGGGCAGGATCAGGAGCGTGTACCCCGTGG | |
| | CCAGCCCCAACGAGTGCAACCAGATGTGCCTGAGCACCCTGATGAAGTGCGA | |
| | CCACTGCGGCGAGACCAGCTGGCAGACCGGCGACTTCGTGAAGGCCACCTG | |
| | CGAGTTCTGCGGCACCGAGAACCTGACCAAGGAGGGCGCCACCACCTGCGG | |
| | CTACCTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAGCGGC | |
| | CTGAAGACCATCCTGAGGAAGGGCGGCAGGACCATCGCCTTCGGCGGCTGC | |
| | GTGTTCAGCTACGTGGGCTGCCACAACAAGTGCGCCTACTGGGTGCCCAGGG | |
| | CCAGCGCCAACATCGGCTGCAACCACACCGGCGTGGTGGGCGAGGGCAGCG | |
| | AGGGCCTGAACGACAACCTGCTGGAGATCCTGCAGAAGGAGAAGGTGAACAT | |
| | CAACATCGTGGGCGACTTCAAGCTGAACGAGGAGATCGCCATCATCCTGGCCA | |
| | GCTTCAGCGCCAGCACCAGCGCCTTCGTGGAGACCGTGAAGGGCCTGGACTA | |
| | CAAGGCCTTCAAGCAGATCGTGGAGAGCTGCGGCAACTTCAAGGTGACCAAG | |
| | GGCAAGGCCAAGAAGGGCGCCTGGAACATCGGCGAGCAGAAGAGCATCCTG | |
| | AGCCCCCTGTACGCCTTCGCCAGCGAGGCCGCCAGGGTGGTGAGGAGCATC | |
| | TTCAGCAGGACCCTGGAGACCGCCCAGAACAGCGTGAGGGTGCTGCAGAAG | |
| | GCCGCCATCACCATCCTGGACGGCATCAGCCAGTACAGCCTGAGGCTGATCG | |
| | ACGCCATGATGTTCACCAGCGACCTGGCCACCAACAACCTGGTGGTGATGGC | |
| | CTACATCACCGGCGGCGTGGTGCAGCTGACCAGCCAGTGGCTGACCAACATC | |
| | TTCGGCACCGTGTACGAGAAGCTGAAGCCCGTGCTGGACTGGCTGGAGGAGA | |
| | AGTTCAAGGAGGGCGTGGAGTTCCTGAGGGACGGCTGGGAGATCGTGAAGTT | |
| | CATCAGCACCTGCGCCTGCGAGATCGTGGGCGGCCAGATCGTGACCTGCGCC | |
| | AAGGAGATCAAGGAGAGCGTGCAGACCTTCTTCAAGCTGGTGAACAAGTTCCT | |
| | GGCCCTGTGCGCCGACAGCATCATCATCGGCGGCGCCAAGCTGAAGGCCCT | |
| | GAACCTGGGCGAGACCTTCGTGACCCACAGCAAGGGCCTGTACAGGAAGTGC | |
| | GTGAAGAGCAGGGAGGAGACCGGCCTGCTGATGCCCCTGAAGGCCCCCAAG | |
| | GAGATCATCTTCCTGGAGGGCGAGACCCTGCCCACCGAGGTGCTGACCGAGG | |
| | AGGTGGTGCTGAAGACCGGCGACCTGCAGCCCCTGGAGCAGCCCACCAGCG | |
| | AGGCCGTGGAGGCCCCCCTGGTGGGCACCCCCGTGTGCATCAACGGCCTGA | |
| | TGCTGCTGGAGATCAAGGACACCGAGAAGTACTGCGCCCTGGCCCCCAACAT | |
| | GATGGTGACCAACAACACCTTCACCCTGAAGGGCGGCTGATGA | |
| Pan-CoV | ATGTTCGTGTTCCTGGTGCTGCTGCCCCTGGTGAGCAGCCAGTGCGTGAACC | 78 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #15 | GACCTGTTCCTGCCCTTCTTCAGCAACGTGACCTGGTTCCACGCCATCCACGT | |
| Spike-NS | GAGCGGCACCAACGGCACCAAGAGGTTCGACAACCCCGTGCTGCCCTTCAAC | |
| P6-NSP7- | GACGGCGTGTACTTCGCCAGCACCGAGAAGAGCAACATCATCAGGGGCTGGA | |
| NSP8 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGC | |
| | CGTGAAGAGGACCATCAAGGGCACCCACCACTGGCTGCTGCTGACCATCCTG | |
| | ACCAGCCTGCTGGTGCTGGTGCAGAGCACCCAGTGGAGCCTGTTCTTCTTCC | |
| | TGTACGAGAACGCCTTCCTGCCCTTCGCCATGGGCATCATCGCCATGAGCGCC | |
| | TTCGCCATGATGTTCGTGAAGCACAAGCACGCCTTCCTGTGCCTGTTCCTGCT | |
| | GCCCAGCCTGGCCACCGTGGCCTACTTCAACATGGTGTACATGCCCGCCAGC | |
| | TGGGTGATGAGGATCATGACCTGGCTGGACATGGTGGACACCAGCCTGAGCG | |
| | GCTTCAAGCTGAAGGACTGCGTGATGTACGCCAGCGCCGTGGTGCTGCTGAT | |
| | CCTGATGACCGCCAGGACCGTGTACGACGACGGCGCCAGGAGGGTGTGGAC | |
| | CCTGATGAACGTGCTGACCCTGGTGTACAAGGTGTACTACGGCAACGCCCTG | |
| | GACCAGGCCATCAGCATGTGGGCCCTGATCATCAGCGTGACCAGCAACTACA | |
| | GCGGCGTGGTGACCACCGTGATGTTCCTGGCCAGGGGCATCGTGTTCATGTG | |
| | CGTGGAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCTGTTC | |
| | TGCCTGCTGAACAGGTACTTCAGGCTGACCCTGGGCGTGTACGACTACCTGG | |
| | TGAGCACCCAGGAGTTCAGGTACATGAACAGCCAGGGCCTGCTGCCCCCCAA | |
| | GAACAGCATCGACGCCTTCAAGCTGAACATCAAGCTGCTGGGCGTGGGCGGC | |
| | AAGCCCTGCATCAAGGTGGCCACCGTGCAGGGAAGCGGAGAGGGCAGGGGA | |
| | AGTCTTCTAACATGCGGGGACGTGGAGGAAAATCCCGGCCCCAGCAAGATGA | |
| | GCGACGTGAAGTGCACCAGCGTGGTGCTGCTGAGCGTGCTGCAGCAGCTGA | |
| | GGGTGGAGAGCAGCAGCAAGCTGTGGGCCCAGTGCGTGCAGCTGCACAACG | |
| | ACATCCTGCTGGCCAAGGACACCACCGAGGCCTTCGAGAAGATGGTGAGCCT | |
| | GCTGAGCGTGCTGCTGAGCATGCAGGGCGCCGTGGACATCAACAAGCTGTGC | |
| | GAGGAGATGCTGGACAACAGGGCCACCCTGCAGGGAAGCGGACAGTGTACTA | |
| | ATTATGCTCTCTTGAAATTGGCTGGAGATGTTGAGAGCAACCCAGGTCCCGCC | |
| | ATCGCCAGCGAGTTCAGCAGCCTGCCCAGCTACGCCGCCTTCGCCACCGCCC | |
| | AGGAGGCCTACGAGCAGGCCGTGGCCAACGGCGACAGCGAGGTGGTGCTGA | |
| | AGAAGCTGAAGAAGAGCCTGAACGTGGCCAAGAGCGAGTTCGACAGGGACG | |
| | CCGCCATGCAGAGGAAGCTGGAGAAGATGGCCGACCAGGCCATGACCCAGAT | |
| | GTACAAGCAGGCCAGGAGCGAGGACAAGAGGGCCAAGGTGACCAGCGCCAT | |
| | GCAGACCATGCTGTTCACCATGCTGAGGAAGCTGGACAACGACGCCCTGAAC | |
| | AACATCATCAACAACGCCAGGGACGGCTGCGTGCCCCTGAACATCATCCCCCT | |
| | GACCACCGCCGCCAAGCTGATGGTGGTGATCCCCGACTACAACACCTACAAG | |
| | AACACCTGCGACGGCACCACCTTCACCTACGCCAGCGCCCTGTGGGAGATCC | |
| | AGCAGGTGGTGGACGCCGACAGCAAGATCGTGCAGCTGAGCGAGATCAGCAT | |
| | GGACAACAGCCCCAACCTGGCCTGGCCCCTGATCGTGACCGCCCTGAGGGC | |
| | CAACAGCGCCGTGAAGCTGCAGTGATGA | |
| Pan-CoV | ATGTTCGTGTTCCTGGTGCTGCTGCCCCTGGTGAGCAGCCAGTGCGTGAACC | 79 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #16 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| NSP6-NS | GACGGCGTGTACTTCGCCAGCACCGAGAAGAGCAACATCATCAGGGGCTGGA | |
| P7-NSP8 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGC | |
| | CGTGAAGAGGACCATCAAGGGCACCCACCACTGGCTGCTGCTGACCATCCTG | |
| | ACCAGCCTGCTGGTGCTGGTGCAGAGCACCCAGTGGAGCCTGTTCTTCTTCC | |
| | TGTACGAGAACGCCTTCCTGCCCTTCGCCATGGGCATCATCGCCATGAGCGCC | |
| | TTCGCCATGATGTTCGTGAAGCACAAGCACGCCTTCCTGTGCCTGTTCCTGCT | |
| | GCCCAGCCTGGCCACCGTGGCCTACTTCAACATGGTGTACATGCCCGCCAGC | |
| | TGGGTGATGAGGATCATGACCTGGCTGGACATGGTGGACACCAGCCTGAGCG | |
| | GCTTCAAGCTGAAGGACTGCGTGATGTACGCCAGCGCCGTGGTGCTGCTGAT | |
| | CCTGATGACCGCCAGGACCGTGTACGACGACGGCGCCAGGAGGGTGTGGAC | |
| | CCTGATGAACGTGCTGACCCTGGTGTACAAGGTGTACTACGGCAACGCCCTG | |
| | GACCAGGCCATCAGCATGTGGGCCCTGATCATCAGCGTGACCAGCAACTACA | |
| | GCGGCGTGGTGACCACCGTGATGTTCCTGGCCAGGGGCATCGTGTTCATGTG | |
| | CGTGGAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCTGTTC | |
| | TGCCTGCTGAACAGGTACTTCAGGCTGACCCTGGGCGTGTACGACTACCTGG | |
| | TGAGCACCCAGGAGTTCAGGTACATGAACAGCCAGGGCCTGCTGCCCCCCAA | |
| | GAACAGCATCGACGCCTTCAAGCTGAACATCAAGCTGCTGGGCGTGGGCGGC | |
| | AAGCCCTGCATCAAGGTGGCCACCGTGCAGGGAAGCGGAGAGGGCAGGGGA | |
| | AGTCTTCTAACATGCGGGGACGTGGAGGAAAATCCCGGCCCCAGCAAGATGA | |
| | GCGACGTGAAGTGCACCAGCGTGGTGCTGCTGAGCGTGCTGCAGCAGCTGA | |
| | GGGTGGAGAGCAGCAGCAAGCTGTGGGCCCAGTGCGTGCAGCTGCACAACG | |
| | ACATCCTGCTGGCCAAGGACACCACCGAGGCCTTCGAGAAGATGGTGAGCCT | |
| | GCTGAGCGTGCTGCTGAGCATGCAGGGCGCCGTGGACATCAACAAGCTGTGC | |
| | GAGGAGATGCTGGACAACAGGGCCACCCTGCAGGGAAGCGGACAGTGTACTA | |
| | ATTATGCTCTCTTGAAATTGGCTGGAGATGTTGAGAGCAACCCAGGTCCCGCC | |
| | ATCGCCAGCGAGTTCAGCAGCCTGCCCAGCTACGCCGCCTTCGCCACCGCCC | |
| | AGGAGGCCTACGAGCAGGCCGTGGCCAACGGCGACAGCGAGGTGGTGCTGA | |
| | AGAAGCTGAAGAAGAGCCTGAACGTGGCCAAGAGCGAGTTCGACAGGGACG | |
| | CCGCCATGCAGAGGAAGCTGGAGAAGATGGCCGACCAGGCCATGACCCAGAT | |
| | GTACAAGCAGGCCAGGAGCGAGGACAAGAGGGCCAAGGTGACCAGCGCCAT | |
| | GCAGACCATGCTGTTCACCATGCTGAGGAAGCTGGACAACGACGCCCTGAAC | |
| | AACATCATCAACAACGCCAGGGACGGCTGCGTGCCCCTGAACATCATCCCCCT | |
| | GACCACCGCCGCCAAGCTGATGGTGGTGATCCCCGACTACAACACCTACAAG | |
| | AACACCTGCGACGGCACCACCTTCACCTACGCCAGCGCCCTGTGGGAGATCC | |
| | AGCAGGTGGTGGACGCCGACAGCAAGATCGTGCAGCTGAGCGAGATCAGCAT | |
| | GGACAACAGCCCCAACCTGGCCTGGCCCCTGATCGTGACCGCCCTGAGGGC | |
| | CAACAGCGCCGTGAAGCTGCAGTGATGA | |
| Pan-CoV | ATGTTCGTGTTCCTGGTGCTGCTGCCCCTGGTGAGCAGCCAGTGCGTGAACC | 80 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #17 | GACCTGTTCCTGCCCTTCTTCAGCAACGTGACCTGGTTCCACGCCATCCACGT | |
| Spike-NS | GAGCGGCACCAACGGCACCAAGAGGTTCGACAACCCCGTGCTGCCCTTCAAC | |
| P9-NSP1 | GACGGCGTGTACTTCGCCAGCACCGAGAAGAGCAACATCATCAGGGGCTGGA | |
| 0-NSP13 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAACAA | |
| | CGAGCTGAGCCCCGTGGCCCTGAGGCAGATGAGCTGCGCCGCCGGCACCAC | |
| | CCAGACCGCCTGCACCGACGACAACGCCCTGGCCTACTACAACACCACCAAG | |
| | GGCGGCAGGTTCGTGCTGGCCCTGCTGAGCGACCTGCAGGACCTGAAGTGG | |
| | GCCAGGTTCCCCAAGAGCGACGGCACCGGCACCATCTACACCGAGCTGGAG | |
| | CCCCCCTGCAGGTTCGTGACCGACACCCCCAAGGGCCCCAAGGTGAAGTACC | |
| | TGTACTTCATCAAGGGCCTGAACAACCTGAACAGGGGCATGGTGCTGGGCAG | |
| | CCTGGCCGCCACCGTGAGGCTGCAGGGAAGCGGAGAGGGCAGGGGAAGTC | |
| | TTCTAACATGCGGGGACGTGGAGGAAAATCCCGGCCCCGCCGGCAACGCCAC | |
| | CGAGGTGCCCGCCAACAGCACCGTGCTGAGCTTCTGCGCCTTCGCCGTGGA | |
| | CGCCGCCAAGGCCTACAAGGACTACCTGGCCAGCGGCGGCCAGCCCATCAC | |
| | CAACTGCGTGAAGATGCTGTGCACCCACACCGGCACCGGCCAGGCCATCACC | |
| | GTGACCCCCGAGGCCAACATGGACCAGGAGAGCTTCGGCGGCGCCAGCTGC | |
| | TGCCTGTACTGCAGGTGCCACATCGACCACCCCAACCCCAAGGGCTTCTGCG | |
| | ACCTGAAGGGCAAGTACGTGCAGATCCCCACCACCTGCGCCAACGACCCCGT | |
| | GGGCTTCACCCTGAAGAACACCGTGTGCACCGTGTGCGGCATGTGGAAGGGC | |
| | TACGGCTGCAGCTGCGACCAGCTGAGGGAGCCCATGCTGCAGGGAAGCGGA | |
| | CAGTGTACTAATTATGCTCTCTTGAAATTGGCTGGAGATGTTGAGAGCAACCCA | |
| | GGTCCCGCCGTGGGCGCCTGCGTGCTGTGCAACAGCCAGACCAGCCTGAGG | |
| | TGCGGCGCCTGCATCAGGAGGCCCTTCCTGTGCTGCAAGTGCTGCTACGACC | |
| | ACGTGATCAGCACCAGCCACAAGCTGGTGCTGAGCGTGAACCCCTACGTGTG | |
| | CAACGCCCCCGGCTGCGACGTGACCGACGTGACCCAGCTGTACCTGGGCGG | |
| | CATGAGCTACTACTGCAAGAGCCACAAGCCCCCCATCAGCTTCCCCCTGTGCG | |
| | CCAACGGCCAGGTGTTCGGCCTGTACAAGAACACCTGCGTGGGCAGCGACAA | |
| | CGTGACCGACTTCAACGCCATCGCCACCTGCGACTGGACCAACGCCGGCGAC | |
| | TACATCCTGGCCAACACCTGCACCGAGAGGCTGAAGCTGTTCGCCGCCGAGA | |
| | CCCTGAAGGCCACCGAGGAGACCTTCAAGCTGAGCTACGGCATCGCCACCGT | |
| | GAGGGAGGTGCTGAGCGACAGGGAGCTGCACCTGAGCTGGGAGGTGGGCAA | |
| | GCCCAGGCCCCCCCTGAACAGGAACTACGTGTTCACCGGCTACAGGGTGACC | |
| | AAGAACAGCAAGGTGCAGATCGGCGAGTACACCTTCGAGAAGGGCGACTACG | |
| | GCGACGCCGTGGTGTACAGGGGCACCACCACCTACAAGCTGAACGTGGGCG | |
| | ACTACTTCGTGCTGACCAGCCACACCGTGATGCCCCTGAGCGCCCCCACCCT | |
| | GGTGCCCCAGGAGCACTACGTGAGGATCACCGGCCTGTACCCCACCCTGAAC | |
| | ATCAGCGACGAGTTCAGCAGCAACGTGGCCAACTACCAGAAGGTGGGCATGC | |
| | AGAAGTACAGCACCCTGCAGGGCCCCCCCGGCACCGGCAAGAGCCACTTCG | |
| | CCATCGGCCTGGCCCTGTACTACCCCAGCGCCAGGATCGTGTACACCGCCTG | |
| | CAGCCACGCCGCCGTGGACGCCCTGTGCGAGAAGGCCCTGAAGTACCTGCC | |
| | CATCGACAAGTGCAGCAGGATCATCCCCGCCAGGGCCAGGGTGGAGTGCTTC | |
| | GACAAGTTCAAGGTGAACAGCACCCTGGAGCAGTACGTGTTCTGCACCGTGA | |
| | ACGCCCTGCCCGAGACCACCGCCGACATCGTGGTGTTCGACGAGATCAGCAT | |
| | GGCCACCAACTACGACCTGAGCGTGGTGAACGCCAGGCTGAGGGCCAAGCA | |
| | CTACGTGTACATCGGCGACCCCGCCCAGCTGCCCGCCCCCAGGACCCTGCTG | |
| | ACCAAGGGCACCCTGGAGCCCGAGTACTTCAACAGCGTGTGCAGGCTGATGA | |
| | AGACCATCGGCCCCGACATGTTCCTGGGCACCTGCAGGAGGTGCCCCGCCG | |
| | AGATCGTGGACACCGTGAGCGCCCTGGTGTACGACAACAAGCTGAAGGCCCA | |
| | CAAGGACAAGAGCGCCCAGTGCTTCAAGATGTTCTACAAGGGCGTGATCACC | |
| | CACGACGTGAGCAGCGCCATCAACAGGCCCCAGATCGGCGTGGTGAGGGAG | |
| | TTCCTGACCAGGAACCCCGCCTGGAGGAAGGCCGTGTTCATCAGCCCCTACA | |
| | ACAGCCAGAACGCCGTGGCCAGCAAGATCCTGGGCCTGCCCACCCAGACCG | |
| | TGGACAGCAGCCAGGGCAGCGAGTACGACTACGTGATCTTCACCCAGACCAC | |
| | CGAGACCGCCCACAGCTGCAACGTGAACAGGTTCAACGTGGCCATCACCAGG | |
| | GCCAAGGTGGGCATCCTGTGCATCATGAGCGACAGGGACCTGTACGACAAGC | |
| | TGCAGTTCACCAGCCTGGAGATCCCCAGGAGGAACGTGGCCACCCTGCAGTG | |
| | ATGA | |
| Pan-CoV | ATGTTCGTGTTCCTGGTGCTGCTGCCCCTGGTGAGCAGCCAGTGCGTGAACC | 81 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #18 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| NSP9-NS | GACGGCGTGTACTTCGCCAGCACCGAGAAGAGCAACATCATCAGGGGCTGGA | |
| P10-NSP | TCTTCGGCACCACCCTGGACAGCAAGACCCAGAGCCTGCTGATCGTGAACAA | |
| 13 | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAACAA | |
| | CGAGCTGAGCCCCGTGGCCCTGAGGCAGATGAGCTGCGCCGCCGGCACCAC | |
| | CCAGACCGCCTGCACCGACGACAACGCCCTGGCCTACTACAACACCACCAAG | |
| | GGCGGCAGGTTCGTGCTGGCCCTGCTGAGCGACCTGCAGGACCTGAAGTGG | |
| | GCCAGGTTCCCCAAGAGCGACGGCACCGGCACCATCTACACCGAGCTGGAG | |
| | CCCCCCTGCAGGTTCGTGACCGACACCCCCAAGGGCCCCAAGGTGAAGTACC | |
| | TGTACTTCATCAAGGGCCTGAACAACCTGAACAGGGGCATGGTGCTGGGCAG | |
| | CCTGGCCGCCACCGTGAGGCTGCAGGGAAGCGGAGAGGGCAGGGGAAGTC | |
| | TTCTAACATGCGGGGACGTGGAGGAAAATCCCGGCCCCGCCGGCAACGCCAC | |
| | CGAGGTGCCCGCCAACAGCACCGTGCTGAGCTTCTGCGCCTTCGCCGTGGA | |
| | CGCCGCCAAGGCCTACAAGGACTACCTGGCCAGCGGCGGCCAGCCCATCAC | |
| | CAACTGCGTGAAGATGCTGTGCACCCACACCGGCACCGGCCAGGCCATCACC | |
| | GTGACCCCCGAGGCCAACATGGACCAGGAGAGCTTCGGCGGCGCCAGCTGC | |
| | TGCCTGTACTGCAGGTGCCACATCGACCACCCCAACCCCAAGGGCTTCTGCG | |
| | ACCTGAAGGGCAAGTACGTGCAGATCCCCACCACCTGCGCCAACGACCCCGT | |
| | GGGCTTCACCCTGAAGAACACCGTGTGCACCGTGTGCGGCATGTGGAAGGGC | |
| | TACGGCTGCAGCTGCGACCAGCTGAGGGAGCCCATGCTGCAGGGAAGCGGA | |
| | CAGTGTACTAATTATGCTCTCTTGAAATTGGCTGGAGATGTTGAGAGCAACCCA | |
| | GGTCCCGCCGTGGGCGCCTGCGTGCTGTGCAACAGCCAGACCAGCCTGAGG | |
| | TGCGGCGCCTGCATCAGGAGGCCCTTCCTGTGCTGCAAGTGCTGCTACGACC | |
| | ACGTGATCAGCACCAGCCACAAGCTGGTGCTGAGCGTGAACCCCTACGTGTG | |
| | CAACGCCCCCGGCTGCGACGTGACCGACGTGACCCAGCTGTACCTGGGCGG | |
| | CATGAGCTACTACTGCAAGAGCCACAAGCCCCCCATCAGCTTCCCCCTGTGCG | |
| | CCAACGGCCAGGTGTTCGGCCTGTACAAGAACACCTGCGTGGGCAGCGACAA | |
| | CGTGACCGACTTCAACGCCATCGCCACCTGCGACTGGACCAACGCCGGCGAC | |
| | TACATCCTGGCCAACACCTGCACCGAGAGGCTGAAGCTGTTCGCCGCCGAGA | |
| | CCCTGAAGGCCACCGAGGAGACCTTCAAGCTGAGCTACGGCATCGCCACCGT | |
| | GAGGGAGGTGCTGAGCGACAGGGAGCTGCACCTGAGCTGGGAGGTGGGCAA | |
| | GCCCAGGCCCCCCCTGAACAGGAACTACGTGTTCACCGGCTACAGGGTGACC | |
| | AAGAACAGCAAGGTGCAGATCGGCGAGTACACCTTCGAGAAGGGCGACTACG | |
| | GCGACGCCGTGGTGTACAGGGGCACCACCACCTACAAGCTGAACGTGGGCG | |
| | ACTACTTCGTGCTGACCAGCCACACCGTGATGCCCCTGAGCGCCCCCACCCT | |
| | GGTGCCCCAGGAGCACTACGTGAGGATCACCGGCCTGTACCCCACCCTGAAC | |
| | ATCAGCGACGAGTTCAGCAGCAACGTGGCCAACTACCAGAAGGTGGGCATGC | |
| | AGAAGTACAGCACCCTGCAGGGCCCCCCCGGCACCGGCAAGAGCCACTTCG | |
| | CCATCGGCCTGGCCCTGTACTACCCCAGCGCCAGGATCGTGTACACCGCCTG | |
| | CAGCCACGCCGCCGTGGACGCCCTGTGCGAGAAGGCCCTGAAGTACCTGCC | |
| | CATCGACAAGTGCAGCAGGATCATCCCCGCCAGGGCCAGGGTGGAGTGCTTC | |
| | GACAAGTTCAAGGTGAACAGCACCCTGGAGCAGTACGTGTTCTGCACCGTGA | |
| | ACGCCCTGCCCGAGACCACCGCCGACATCGTGGTGTTCGACGAGATCAGCAT | |
| | GGCCACCAACTACGACCTGAGCGTGGTGAACGCCAGGCTGAGGGCCAAGCA | |
| | CTACGTGTACATCGGCGACCCCGCCCAGCTGCCCGCCCCCAGGACCCTGCTG | |
| | ACCAAGGGCACCCTGGAGCCCGAGTACTTCAACAGCGTGTGCAGGCTGATGA | |
| | AGACCATCGGCCCCGACATGTTCCTGGGCACCTGCAGGAGGTGCCCCGCCG | |
| | AGATCGTGGACACCGTGAGCGCCCTGGTGTACGACAACAAGCTGAAGGCCCA | |
| | CAAGGACAAGAGCGCCCAGTGCTTCAAGATGTTCTACAAGGGCGTGATCACC | |
| | CACGACGTGAGCAGCGCCATCAACAGGCCCCAGATCGGCGTGGTGAGGGAG | |
| | TTCCTGACCAGGAACCCCGCCTGGAGGAAGGCCGTGTTCATCAGCCCCTACA | |
| | ACAGCCAGAACGCCGTGGCCAGCAAGATCCTGGGCCTGCCCACCCAGACCG | |
| | TGGACAGCAGCCAGGGCAGCGAGTACGACTACGTGATCTTCACCCAGACCAC | |
| | CGAGACCGCCCACAGCTGCAACGTGAACAGGTTCAACGTGGCCATCACCAGG | |
| | GCCAAGGTGGGCATCCTGTGCATCATGAGCGACAGGGACCTGTACGACAAGC | |
| | TGCAGTTCACCAGCCTGGAGATCCCCAGGAGGAACGTGGCCACCCTGCAGTG | |
| | ATGA | |
| Pan-CoV | ATGTTCGTGTTCCTGGTGCTGCTGCCCCTGGTGAGCAGCCAGTGCGTGAACC | 82 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #19 | GACCTGTTCCTGCCCTTCTTCAGCAACGTGACCTGGTTCCACGCCATCCACGT | |
| Spike-NS | GAGCGGCACCAACGGCACCAAGAGGTTCGACAACCCCGTGCTGCCCTTCAAC | |
| P14-NSP | GACGGCGTGTACTTCGCCAGCACCGAGAAGAGCAACATCATCAGGGGCTGGA | |
| 15 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCGA | |
| | GAACGTGACCGGCCTGTTCAAGGACTGCAGCAAGGTGATCACCGGCCTGCAC | |
| | CCCACCCAGGCCCCCACCCACCTGAGCGTGGACACCAAGTTCAAGACCGAG | |
| | GGCCTGTGCGTGGACATCCCCGGCATCCCCAAGGACATGACCTACAGGAGGC | |
| | TGATCAGCATGATGGGCTTCAAGATGAACTACCAGGTGAACGGCTACCCCAAC | |
| | ATGTTCATCACCAGGGAGGAGGCCATCAGGCACGTGAGGGCCTGGATCGGCT | |
| | TCGACGTGGAGGGCTGCCACGCCACCAGGGAGGCCGTGGGCACCAACCTGC | |
| | CCCTGCAGCTGGGCTTCAGCACCGGCGTGAACCTGGTGGCCGTGCCCACCG | |
| | GCTACGTGGACACCCCCAACAACACCGACTTCAGCAGGGTGAGCGCCAAGCC | |
| | CCCCCCCGGCGACCAGTTCAAGCACCTGATCCCCCTGATGTACAAGGGCCTG | |
| | CCCTGGAACGTGGTGAGGATCAAGATCGTGCAGATGCTGAGCGACACCCTGA | |
| | AGAACCTGAGCGACAGGGTGGTGTTCGTGCTGTGGGCCCACGGCTTCGAGC | |
| | TGACCAGCATGAAGTACTTCGTGAAGATCGGCCCCGAGAGGACCTGCTGCCT | |
| | GTGCGACAGGAGGGCCACCTGCTTCAGCACCGCCAGCGACACCTACGCCTG | |
| | CTGGCACCACAGCATCGGCTTCGACTACGTGTACAACCCCTTCATGATCGACG | |
| | TGCAGCAGTGGGGCTTCACCGGCAACCTGCAGAGCAACCACGACCTGTACTG | |
| | CCAGGTGCACGGCAACGCCCACGTGGCCAGCTGCGACGCCATCATGACCAG | |
| | GTGCCTGGCCGTGCACGAGTGCTTCGTGAAGAGGGTGGACTGGACCATCGA | |
| | GTACCCCATCATCGGCGACGAGCTGAAGATCAACGCCGCCTGCAGGAAGGTG | |
| | CAGCACATGGTGGTGAAGGCCGCCCTGCTGGCCGACAAGTTCCCCGTGCTGC | |
| | ACGACATCGGCAACCCCAAGGCCATCAAGTGCGTGCCCCAGGCCGACGTGGA | |
| | GTGGAAGTTCTACGACGCCCAGCCCTGCAGCGACAAGGCCTACAAGATCGAG | |
| | GAGCTGTTCTACAGCTACGCCACCCACAGCGACAAGTTCACCGACGGCGTGT | |
| | GCCTGTTCTGGAACTGCAACGTGGACAGGTACCCCGCCAACAGCATCGTGTG | |
| | CAGGTTCGACACCAGGGTGCTGAGCAACCTGAACCTGCCCGGCTGCGACGG | |
| | CGGCAGCCTGTACGTGAACAAGCACGCCTTCCACACCCCCGCCTTCGACAAG | |
| | AGCGCCTTCGTGAACCTGAAGCAGCTGCCCTTCTTCTACTACAGCGACAGCCC | |
| | CTGCGAGAGCCACGGCAAGCAGGTGGTGAGCGACATCGACTACGTGCCCCT | |
| | GAAGAGCGCCACCTGCATCACCAGGTGCAACCTGGGCGGCGCCGTGTGCAG | |
| | GCACCACGCCAACGAGTACAGGCTGTACCTGGACGCCTACAACATGATGATCA | |
| | GCGCCGGCTTCAGCCTGTGGGTGTACAAGCAGTTCGACACCTACAACCTGTG | |
| | GAACACCTTCACCAGGCTGCAGGGAAGCGGAGAGGGCAGGGGAAGTCTTCTA | |
| | ACATGCGGGGACGTGGAGGAAAATCCCGGCCCCAGCCTGGAGAACGTGGCC | |
| | TTCAACGTGGTGAACAAGGGCCACTTCGACGGCCAGCAGGGCGAGGTGCCC | |
| | GTGAGCATCATCAACAACACCGTGTACACCAAGGTGGACGGCGTGGACGTGG | |
| | AGCTGTTCGAGAACAAGACCACCCTGCCCGTGAACGTGGCCTTCGAGCTGTG | |
| | GGCCAAGAGGAACATCAAGCCCGTGCCCGAGGTGAAGATCCTGAACAACCTG | |
| | GGCGTGGACATCGCCGCCAACACCGTGATCTGGGACTACAAGAGGGACGCCC | |
| | CCGCCCACATCAGCACCATCGGCGTGTGCAGCATGACCGACATCGCCAAGAA | |
| | GCCCACCGAGACCATCTGCGCCCCCCTGACCGTGTTCTTCGACGGCAGGGTG | |
| | GACGGCCAGGTGGACCTGTTCAGGAACGCCAGGAACGGCGTGCTGATCACC | |
| | GAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTAC | |
| | TACAAGAAGGTGGACGGCGTGGTGCAGCAGCTGCCCGAGACCTACTTCACCC | |
| | AGAGCAGGAACCTGCAGGAGTTCAAGCCCAGGAGCCAGATGGAGATCGACTT | |
| | CCTGGAGCTGGCCATGGACGAGTTCATCGAGAGGTACAAGCTGGAGGGCTAC | |
| | GCCTTCGAGCACATCGTGTACGGCGACTTCAGCCACAGCCAGCTGGGCGGCC | |
| | TGCACCTGCTGATCGGCCTGGCCAAGAGGTTCAAGGAGAGCCCCTTCGAGCT | |
| | GGAGGACTTCATCCCCATGGACAGCACCGTGAAGAACTACTTCATCACCGACG | |
| | CCCAGACCGGCAGCAGCAAGTGCGTGTGCAGCGTGATCGACCTGCTGCTGG | |
| | ACGACTTCGTGGAGATCATCAAGAGCCAGGACCTGAGCGTGGTGAGCAAGGT | |
| | GGTGAAGGTGACCATCGACTACACCGAGATCAGCTTCATGCTGTGGTGCAAGG | |
| | ACGGCCACGTGGAGACCTTCTACCCCAAGCTGCAGTGATGA | |
| Pan-CoV | ATGTTCGTGTTCCTGGTGCTGCTGCCCCTGGTGAGCAGCCAGTGCGTGAACC | 83 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #20 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| NSP14-N | GACGGCGTGTACTTCGCCAGCACCGAGAAGAGCAACATCATCAGGGGCTGGA | |
| SP15 | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCGA | |
| | GAACGTGACCGGCCTGTTCAAGGACTGCAGCAAGGTGATCACCGGCCTGCAC | |
| | CCCACCCAGGCCCCCACCCACCTGAGCGTGGACACCAAGTTCAAGACCGAG | |
| | GGCCTGTGCGTGGACATCCCCGGCATCCCCAAGGACATGACCTACAGGAGGC | |
| | TGATCAGCATGATGGGCTTCAAGATGAACTACCAGGTGAACGGCTACCCCAAC | |
| | ATGTTCATCACCAGGGAGGAGGCCATCAGGCACGTGAGGGCCTGGATCGGCT | |
| | TCGACGTGGAGGGCTGCCACGCCACCAGGGAGGCCGTGGGCACCAACCTGC | |
| | CCCTGCAGCTGGGCTTCAGCACCGGCGTGAACCTGGTGGCCGTGCCCACCG | |
| | GCTACGTGGACACCCCCAACAACACCGACTTCAGCAGGGTGAGCGCCAAGCC | |
| | CCCCCCCGGCGACCAGTTCAAGCACCTGATCCCCCTGATGTACAAGGGCCTG | |
| | CCCTGGAACGTGGTGAGGATCAAGATCGTGCAGATGCTGAGCGACACCCTGA | |
| | AGAACCTGAGCGACAGGGTGGTGTTCGTGCTGTGGGCCCACGGCTTCGAGC | |
| | TGACCAGCATGAAGTACTTCGTGAAGATCGGCCCCGAGAGGACCTGCTGCCT | |
| | GTGCGACAGGAGGGCCACCTGCTTCAGCACCGCCAGCGACACCTACGCCTG | |
| | CTGGCACCACAGCATCGGCTTCGACTACGTGTACAACCCCTTCATGATCGACG | |
| | TGCAGCAGTGGGGCTTCACCGGCAACCTGCAGAGCAACCACGACCTGTACTG | |
| | CCAGGTGCACGGCAACGCCCACGTGGCCAGCTGCGACGCCATCATGACCAG | |
| | GTGCCTGGCCGTGCACGAGTGCTTCGTGAAGAGGGTGGACTGGACCATCGA | |
| | GTACCCCATCATCGGCGACGAGCTGAAGATCAACGCCGCCTGCAGGAAGGTG | |
| | CAGCACATGGTGGTGAAGGCCGCCCTGCTGGCCGACAAGTTCCCCGTGCTGC | |
| | ACGACATCGGCAACCCCAAGGCCATCAAGTGCGTGCCCCAGGCCGACGTGGA | |
| | GTGGAAGTTCTACGACGCCCAGCCCTGCAGCGACAAGGCCTACAAGATCGAG | |
| | GAGCTGTTCTACAGCTACGCCACCCACAGCGACAAGTTCACCGACGGCGTGT | |
| | GCCTGTTCTGGAACTGCAACGTGGACAGGTACCCCGCCAACAGCATCGTGTG | |
| | CAGGTTCGACACCAGGGTGCTGAGCAACCTGAACCTGCCCGGCTGCGACGG | |
| | CGGCAGCCTGTACGTGAACAAGCACGCCTTCCACACCCCCGCCTTCGACAAG | |
| | AGCGCCTTCGTGAACCTGAAGCAGCTGCCCTTCTTCTACTACAGCGACAGCCC | |
| | CTGCGAGAGCCACGGCAAGCAGGTGGTGAGCGACATCGACTACGTGCCCCT | |
| | GAAGAGCGCCACCTGCATCACCAGGTGCAACCTGGGCGGCGCCGTGTGCAG | |
| | GCACCACGCCAACGAGTACAGGCTGTACCTGGACGCCTACAACATGATGATCA | |
| | GCGCCGGCTTCAGCCTGTGGGTGTACAAGCAGTTCGACACCTACAACCTGTG | |
| | GAACACCTTCACCAGGCTGCAGGGAAGCGGAGAGGGCAGGGGAAGTCTTCTA | |
| | ACATGCGGGGACGTGGAGGAAAATCCCGGCCCCAGCCTGGAGAACGTGGCC | |
| | TTCAACGTGGTGAACAAGGGCCACTTCGACGGCCAGCAGGGCGAGGTGCCC | |
| | GTGAGCATCATCAACAACACCGTGTACACCAAGGTGGACGGCGTGGACGTGG | |
| | AGCTGTTCGAGAACAAGACCACCCTGCCCGTGAACGTGGCCTTCGAGCTGTG | |
| | GGCCAAGAGGAACATCAAGCCCGTGCCCGAGGTGAAGATCCTGAACAACCTG | |
| | GGCGTGGACATCGCCGCCAACACCGTGATCTGGGACTACAAGAGGGACGCCC | |
| | CCGCCCACATCAGCACCATCGGCGTGTGCAGCATGACCGACATCGCCAAGAA | |
| | GCCCACCGAGACCATCTGCGCCCCCCTGACCGTGTTCTTCGACGGCAGGGTG | |
| | GACGGCCAGGTGGACCTGTTCAGGAACGCCAGGAACGGCGTGCTGATCACC | |
| | GAGGGCAGCGTGAAGGGCCTGCAGCCCAGCGTGGGCCCCAAGCAGGCCAG | |
| | CCTGAACGGCGTGACCCTGATCGGCGAGGCCGTGAAGACCCAGTTCAACTAC | |
| | TACAAGAAGGTGGACGGCGTGGTGCAGCAGCTGCCCGAGACCTACTTCACCC | |
| | AGAGCAGGAACCTGCAGGAGTTCAAGCCCAGGAGCCAGATGGAGATCGACTT | |
| | CCTGGAGCTGGCCATGGACGAGTTCATCGAGAGGTACAAGCTGGAGGGCTAC | |
| | GCCTTCGAGCACATCGTGTACGGCGACTTCAGCCACAGCCAGCTGGGCGGCC | |
| | TGCACCTGCTGATCGGCCTGGCCAAGAGGTTCAAGGAGAGCCCCTTCGAGCT | |
| | GGAGGACTTCATCCCCATGGACAGCACCGTGAAGAACTACTTCATCACCGACG | |
| | CCCAGACCGGCAGCAGCAAGTGCGTGTGCAGCGTGATCGACCTGCTGCTGG | |
| | ACGACTTCGTGGAGATCATCAAGAGCCAGGACCTGAGCGTGGTGAGCAAGGT | |
| | GGTGAAGGTGACCATCGACTACACCGAGATCAGCTTCATGCTGTGGTGCAAGG | |
| | ACGGCCACGTGGAGACCTTCTACCCCAAGCTGCAGTGATGA | |
| Pan-CoV | ATGTTCGTGTTCCTGGTGCTGCTGCCCCTGGTGAGCAGCCAGTGCGTGAACC | 84 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #21 | GACCTGTTCCTGCCCTTCTTCAGCAACGTGACCTGGTTCCACGCCATCCACGT | |
| Spike-NS | GAGCGGCACCAACGGCACCAAGAGGTTCGACAACCCCGTGCTGCCCTTCAAC | |
| P16 | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCAG | |
| | CCAGGCCTGGCAGCCCGGCGTGGCCATGCCCAACCTGTACAAGATGCAGAG | |
| | GATGCTGCTGGAGAAGTGCGACCTGCAGAACTACGGCGACAGCGCCACCCTG | |
| | CCCAAGGGCATCATGATGAACGTGGCCAAGTACACCCAGCTGTGCCAGTACCT | |
| | GAACACCCTGACCCTGGCCGTGCCCTACAACATGAGGGTGATCCACTTCGGC | |
| | GCCGGCAGCGACAAGGGCGTGGCCCCCGGCACCGCCGTGCTGAGGCAGTG | |
| | GCTGCCCACCGGCACCCTGCTGGTGGACAGCGACCTGAACGACTTCGTGAG | |
| | CGACGCCGACAGCACCCTGATCGGCGACTGCGCCACCGTGCACACCGCCAA | |
| | CAAGTGGGACCTGATCATCAGCGACATGTACGACCCCAAGACCAAGAACGTGA | |
| | CCAAGGAGAACGACAGCAAGGAGGGCTTCTTCACCTACATCTGCGGCTTCATC | |
| | CAGCAGAAGCTGGCCCTGGGCGGCAGCGTGGCCATCAAGATCACCGAGCAC | |
| | AGCTGGAACGCCGACCTGTACAAGCTGATGGGCCACTTCGCCTGGTGGACCG | |
| | CCTTCGTGACCAACGTGAACGCCAGCAGCAGCGAGGCCTTCCTGATCGGCTG | |
| | CAACTACCTGGGCAAGCCCAGGGAGCAGATCGACGGCTACGTGATGCACGCC | |
| | AACTACATCTTCTGGAGGAACACCAACCCCATCCAGCTGAGCAGCTACAGCCT | |
| | GTTCGACATGAGCAAGTTCCCCCTGAAGCTGAGGGGCACCGCCGTGATGAGC | |
| | CTGAAGGAGGGCCAGATCAACGACATGATCCTGAGCCTGCTGAGCAAGGGCA | |
| | GGCTGATCATCAGGGAGAACAACAGGGTGGTGATCAGCAGCGACGTGCTGGT | |
| | GAACAACTGATGA | |
| Pan-CoV | ATGTTCGTGTTCCTGGTGCTGCTGCCCCTGGTGAGCAGCCAGTGCGTGAACC | 85 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #22 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| NSP16 | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCAG | |
| | CCAGGCCTGGCAGCCCGGCGTGGCCATGCCCAACCTGTACAAGATGCAGAG | |
| | GATGCTGCTGGAGAAGTGCGACCTGCAGAACTACGGCGACAGCGCCACCCTG | |
| | CCCAAGGGCATCATGATGAACGTGGCCAAGTACACCCAGCTGTGCCAGTACCT | |
| | GAACACCCTGACCCTGGCCGTGCCCTACAACATGAGGGTGATCCACTTCGGC | |
| | GCCGGCAGCGACAAGGGCGTGGCCCCCGGCACCGCCGTGCTGAGGCAGTG | |
| | GCTGCCCACCGGCACCCTGCTGGTGGACAGCGACCTGAACGACTTCGTGAG | |
| | CGACGCCGACAGCACCCTGATCGGCGACTGCGCCACCGTGCACACCGCCAA | |
| | CAAGTGGGACCTGATCATCAGCGACATGTACGACCCCAAGACCAAGAACGTGA | |
| | CCAAGGAGAACGACAGCAAGGAGGGCTTCTTCACCTACATCTGCGGCTTCATC | |
| | CAGCAGAAGCTGGCCCTGGGCGGCAGCGTGGCCATCAAGATCACCGAGCAC | |
| | AGCTGGAACGCCGACCTGTACAAGCTGATGGGCCACTTCGCCTGGTGGACCG | |
| | CCTTCGTGACCAACGTGAACGCCAGCAGCAGCGAGGCCTTCCTGATCGGCTG | |
| | CAACTACCTGGGCAAGCCCAGGGAGCAGATCGACGGCTACGTGATGCACGCC | |
| | AACTACATCTTCTGGAGGAACACCAACCCCATCCAGCTGAGCAGCTACAGCCT | |
| | GTTCGACATGAGCAAGTTCCCCCTGAAGCTGAGGGGCACCGCCGTGATGAGC | |
| | CTGAAGGAGGGCCAGATCAACGACATGATCCTGAGCCTGCTGAGCAAGGGCA | |
| | GGCTGATCATCAGGGAGAACAACAGGGTGGTGATCAGCAGCGACGTGCTGGT | |
| | GAACAACTGATGA | |
| Pan-CoV | ATGTTCGTGTTCCTGGTGCTGCTGCCCCTGGTGAGCAGCCAGTGCGTGAACC | 86 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #23 | GACCTGTTCCTGCCCTTCTTCAGCAACGTGACCTGGTTCCACGCCATCCACGT | |
| Spike-OR | GAGCGGCACCAACGGCACCAAGAGGTTCGACAACCCCGTGCTGCCCTTCAAC | |
| F3a | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCAGGAGGGCCAGGAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCTTCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCGCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | CCTGTTCATGAGGATCTTCACCATCGGCACCGTGACCCTGAAGCAGGGCGAG | |
| | ATCAAGGACGCCACCCCCAGCGACTTCGTGAGGGCCACCGCCACCATCCCCA | |
| | TCCAGGCCAGCCTGCCCTTCGGCTGGCTGATCGTGGGCGTGGCCCTGCTGG | |
| | CCGTGTTCCAGAGCGCCAGCAAGATCATCACCCTGAAGAAGAGGTGGCAGCT | |
| | GGCCCTGAGCAAGGGCGTGCACTTCGTGTGCAACCTGCTGCTGCTGTTCGTG | |
| | ACCGTGTACAGCCACCTGCTGCTGGTGGCCGCCGGCCTGGAGGCCCCCTTC | |
| | CTGTACCTGTACGCCCTGGTGTACTTCCTGCAGAGCATCAACTTCGTGAGGAT | |
| | CATCATGAGGCTGTGGCTGTGCTGGAAGTGCAGGAGCAAGAACCCCCTGCTG | |
| | TACGACGCCAACTACTTCCTGTGCTGGCACACCAACTGCTACGACTACTGCAT | |
| | CCCCTACAACAGCGTGACCAGCAGCATCGTGATCACCAGCGGCGACGGCACC | |
| | ACCAGCCCCATCAGCGAGCACGACTACCAGATCGGCGGCTACACCGAGAAGT | |
| | GGGAGAGCGGCGTGAAGGACTGCGTGGTGCTGCACAGCTACTTCACCAGCG | |
| | ACTACTACCAGCTGTACAGCACCCAGCTGAGCACCGACACCGGCGTGGAGCA | |
| | CGTGACCTTCTTCATCTACAACAAGATCGTGGACGAGCCCGAGGAGCACGTG | |
| | CAGATCCACACCATCGACGGCAGCAGCGGCGTGGTGAACCCCGTGATGGAGC | |
| | CCATCTACGACGAGCCCACCACCACCACCAGCGTGCCCCTGTGATGA | |
| Pan-CoV | ATGTTCGTGTTCCTGGTGCTGCTGCCCCTGGTGAGCAGCCAGTGCGTGAACC | 87 |
| mRNA | TGACCACCAGGACCCAGCTGCCCCCCGCCTACACCAACAGCTTCACCAGGGG | |
| Vaccine | CGTGTACTACCCCGACAAGGTGTTCAGGAGCAGCGTGCTGCACAGCACCCAG | |
| #24 | GACCTGTTCCTGCCCTTCTTCAGCAACGTGACCTGGTTCCACGCCATCCACGT | |
| HexaPro- | GAGCGGCACCAACGGCACCAAGAGGTTCGACAACCCCGTGCTGCCCTTCAAC | |
| ORF3a | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGGA | |
| | CCTGTTCATGAGGATCTTCACCATCGGCACCGTGACCCTGAAGCAGGGCGAG | |
| | ATCAAGGACGCCACCCCCAGCGACTTCGTGAGGGCCACCGCCACCATCCCCA | |
| | TCCAGGCCAGCCTGCCCTTCGGCTGGCTGATCGTGGGCGTGGCCCTGCTGG | |
| | CCGTGTTCCAGAGCGCCAGCAAGATCATCACCCTGAAGAAGAGGTGGCAGCT | |
| | GGCCCTGAGCAAGGGCGTGCACTTCGTGTGCAACCTGCTGCTGCTGTTCGTG | |
| | ACCGTGTACAGCCACCTGCTGCTGGTGGCCGCCGGCCTGGAGGCCCCCTTC | |
| | CTGTACCTGTACGCCCTGGTGTACTTCCTGCAGAGCATCAACTTCGTGAGGAT | |
| | CATCATGAGGCTGTGGCTGTGCTGGAAGTGCAGGAGCAAGAACCCCCTGCTG | |
| | TACGACGCCAACTACTTCCTGTGCTGGCACACCAACTGCTACGACTACTGCAT | |
| | CCCCTACAACAGCGTGACCAGCAGCATCGTGATCACCAGCGGCGACGGCACC | |
| | ACCAGCCCCATCAGCGAGCACGACTACCAGATCGGCGGCTACACCGAGAAGT | |
| | GGGAGAGCGGCGTGAAGGACTGCGTGGTGCTGCACAGCTACTTCACCAGCG | |
| | ACTACTACCAGCTGTACAGCACCCAGCTGAGCACCGACACCGGCGTGGAGCA | |
| | CGTGACCTTCTTCATCTACAACAAGATCGTGGACGAGCCCGAGGAGCACGTG | |
| | CAGATCCACACCATCGACGGCAGCAGCGGCGTGGTGAACCCCGTGATGGAGC | |
| | CCATCTACGACGAGCCCACCACCACCACCAGCGTGCCCCTGTGATGA | |
| Pan-CoV | CTCGACATTGATTATTGACTAGTTATTAATAGTAATCAATTACGGGGTCATTAGTT | 88 |
| Vaccine | CATAGCCCATATATGGAGTTCCGCGTTACATAACTTACGGTAAATGGCCCGCCT | |
| #25 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-Hexa | GTAAACTGCCCACTTGGCAGTACATCAAGTGTATCATATGCCAAGTACGCCCCC | |
| Pro | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGCGGGGCGGGGGCGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCAGGTGG | |
| | GGGTGCCGGGCGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGTTCGTGTTCCTGGTGCTGCTGC | |
| | CCCTGGTGAGCAGCCAGTGCGTGAACCTGACCACCAGGACCCAGCTGCCCC | |
| | CCGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAGC | |
| | AACGTGACCTGGTTCCACGCCATCCACGTGAGCGGCACCAACGGCACCAAGA | |
| | GGTTCGACAACCCCGTGCTGCCCTTCAACGACGGCGTGTACTTCGCCAGCAC | |
| | CGAGAAGAGCAACATCATCAGGGGCTGGATCTTCGGCACCACCCTGGACAGC | |
| | AAGACCCAGAGCCTGCTGATCGTGAACAACGCCACCAACGTGGTGATCAAGG | |
| | TGTGCGAGTTCCAGTTCTGCAACGACCCCTTCCTGGGCGTGTACTACCACAAG | |
| | AACAACAAGAGCTGGATGGAGAGCGAGTTCAGGGTGTACAGCAGCGCCAACA | |
| | ACTGCACCTTCGAGTACGTGAGCCAGCCCTTCCTGATGGACCTGGAGGGCAA | |
| | GCAGGGCAACTTCAAGAACCTGAGGGAGTTCGTGTTCAAGAACATCGACGGC | |
| | TACTTCAAGATCTACAGCAAGCACACCCCCATCAACCTGGTGAGGGACCTGCC | |
| | CCAGGGCTTCAGCGCCCTGGAGCCCCTGGTGGACCTGCCCATCGGCATCAAC | |
| | ATCACCAGGTTCCAGACCCTGCTGGCCCTGCACAGGAGCTACCTGACCCCCG | |
| | GCGACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCAC | |
| | CGACGCCGTGGACTGCGCCCTGGACCCCCTGAGCGAGACCAAGTGCACCCT | |
| | GAAGAGCTTCACCGTGGAGAAGGGCATCTACCAGACCAGCAACTTCAGGGTG | |
| | CAGCCCACCGAGAGCATCGTGAGGTTCCCCAACATCACCAACCTGTGCCCCT | |
| | TCGGCGAGGTGTTCAACGCCACCAGGTTCGCCAGCGTGTACGCCTGGAACAG | |
| | GAAGAGGATCAGCAACTGCGTGGCCGACTACAGCGTGCTGTACAACAGCGCC | |
| | AGCTTCAGCACCTTCAAGTGCTACGGCGTGAGCCCCACCAAGCTGAACGACC | |
| | TGTGCTTCACCAACGTGTACGCCGACAGCTTCGTGATCAGGGGCGACGAGGT | |
| | GAGGCAGATCGCCCCCGGCCAGACCGGCAAGATCGCCGACTACAACTACAAG | |
| | CTGCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGAG | |
| | CAACCTGAAGCCCTTCGAGAGGGACATCAGCACCGAGATCTACCAGGCCGGC | |
| | AGCACCCCCTGCAACGGCGTGGAGGGCTTCAACTGCTACTTCCCCCTGCAGA | |
| | GCTACGGCTTCCAGCCCACCAACGGCGTGGGCTACCAGCCCTACAGGGTGGT | |
| | GGTGCTGAGCTTCGAGCTGCTGCACGCCCCCGCCACCGTGTGCGGCCCCAA | |
| | GAAGAGCACCAACCTGGTGAAGAACAAGTGCGTGAACTTCAACTTCAACGGC | |
| | CTGACCGGCACCGGCGTGCTGACCGAGAGCAACAAGAAGTTCCTGCCCTTCC | |
| | AGCAGTTCGGCAGGGACATCGCCGACACCACCGACGCCGTGAGGGACCCCC | |
| | AGACCCTGGAGATCCTGGACATCACCCCCTGCAGCTTCGGCGGCGTGAGCGT | |
| | GATCACCCCCGGCACCAACACCAGCAACCAGGTGGCCGTGCTGTACCAGGAC | |
| | GTGAACTGCACCGAGGTGCCCGTGGCCATCCACGCCGACCAGCTGACCCCC | |
| | ACCTGGAGGGTGTACAGCACCGGCAGCAACGTGTTCCAGACCAGGGCCGGC | |
| | TGCCTGATCGGCGCCGAGCACGTGAACAACAGCTACGAGTGCGACATCCCCA | |
| | TCGGCGCCGGCATCTGCGCCAGCTACCAGACCCAGACCAACAGCCCCGGCA | |
| | GCGCCAGCAGCGTGGCCAGCCAGAGCATCATCGCCTACACCATGAGCCTGGG | |
| | CGCCGAGAACAGCGTGGCCTACAGCAACAACAGCATCGCCATCCCCACCAAC | |
| | TTCACCATCAGCGTGACCACCGAGATCCTGCCCGTGAGCATGACCAAGACCA | |
| | GCGTGGACTGCACCATGTACATCTGCGGCGACAGCACCGAGTGCAGCAACCT | |
| | GCTGCTGCAGTACGGCAGCTTCTGCACCCAGCTGAACAGGGCCCTGACCGGC | |
| | ATCGCCGTGGAGCAGGACAAGAACACCCAGGAGGTGTTCGCCCAGGTGAAG | |
| | CAGATCTACAAGACCCCCCCCATCAAGGACTTCGGCGGCTTCAACTTCAGCCA | |
| | GATCCTGCCCGACCCCAGCAAGCCCAGCAAGAGGAGCCCCATCGAGGACCT | |
| | GCTGTTCAACAAGGTGACCCTGGCCGACGCCGGCTTCATCAAGCAGTACGGC | |
| | GACTGCCTGGGCGACATCGCCGCCAGGGACCTGATCTGCGCCCAGAAGTTCA | |
| | ACGGCCTGACCGTGCTGCCCCCCCTGCTGACCGACGAGATGATCGCCCAGTA | |
| | CACCAGCGCCCTGCTGGCCGGCACCATCACCAGCGGCTGGACCTTCGGCGC | |
| | CGGCCCCGCCCTGCAGATCCCCTTCCCCATGCAGATGGCCTACAGGTTCAAC | |
| | GGCATCGGCGTGACCCAGAACGTGCTGTACGAGAACCAGAAGCTGATCGCCA | |
| | ACCAGTTCAACAGCGCCATCGGCAAGATCCAGGACAGCCTGAGCAGCACCCC | |
| | CAGCGCCCTGGGCAAGCTGCAGGACGTGGTGAACCAGAACGCCCAGGCCCT | |
| | GAACACCCTGGTGAAGCAGCTGAGCAGCAACTTCGGCGCCATCAGCAGCGTG | |
| | CTGAACGACATCCTGAGCAGGCTGGACCCCCCCGAGGCCGAGGTGCAGATC | |
| | GACAGGCTGATCACCGGCAGGCTGCAGAGCCTGCAGACCTACGTGACCCAGC | |
| | AGCTGATCAGGGCCGCCGAGATCAGGGCCAGCGCCAACCTGGCCGCCACCA | |
| | AGATGAGCGAGTGCGTGCTGGGCCAGAGCAAGAGGGTGGACTTCTGCGGCA | |
| | AGGGCTACCACCTGATGAGCTTCCCCCAGAGCGCCCCCCACGGCGTGGTGTT | |
| | CCTGCACGTGACCTACGTGCCCGCCCAGGAGAAGAACTTCACCACCGCCCCC | |
| | GCCATCTGCCACGACGGCAAGGCCCACTTCCCCAGGGAGGGCGTGTTCGTG | |
| | AGCAACGGCACCCACTGGTTCGTGACCCAGAGGAACTTCTACGAGCCCCAGA | |
| | TCATCACCACCGACAACACCTTCGTGAGCGGCAACTGCGACGTGGTGATCGG | |
| | CATCGTGAACAACACCGTGTACGACCCCCTGCAGCCCGAGCTGGACAGCTTC | |
| | AAGGAGGAGCTGGACAAGTACTTCAAGAACCACACCAGCCCCGACGTGGACC | |
| | TGGGCGACATCAGCGGCATCAACGCCAGCGTGGTGAACATCCAGAAGGAGAT | |
| | CGACAGGCTGAACGAGGTGGCCAAGAACCTGAACGAGAGCCTGATCGACCTG | |
| | CAGGAGCTGGGCAAGTACGAGCAGTACATCAAGTGGCCCTGGTACATCTGGC | |
| | TGGGCTTCATCGCCGGCCTGATCGCCATCGTGATGGTGACCATCATGCTGTGC | |
| | TGCATGACCAGCTGCTGCAGCTGCCTGAAGGGCTGCTGCAGCTGCGGCAGCT | |
| | GCTGCAAGTTCGACGAGGACGACAGCGAGCCCGTGCTGAAGGGCGTGAAGC | |
| | TGCACTACACCTGATGACTCGAGCTGGTACTGCATGCACGCAATGCTAGCTGC | |
| | CCCTTTCCCGTCCTGGGTACCCCGAGTCTCCCCCGACCTCGGGTCCCAGGTA | |
| | TGCTCCCACCTCCACCTGCCCCACTCACCACCTCTGCTAGTTCCAGACACCTC | |
| | CCAAGCACGCAGCAATGCAGCTCAAAACGCTTAGCCTAGCCACACCCCCACG | |
| | GGAAACAGCAGTGATTAACCTTTAGCAATAAACGAAAGTTTAACTAAGCTATACT | |
| | AACCCCAGGGTTGGTCAATTTCGTGCCAGCCACACCCTGGAGCTAGCAAAAAA | |
| | AA | |
| Pan-CoV | CTCGACATTGATTATTGACTAGTTATTAATAGTAATCAATTACGGGGTCATTAGTT | 89 |
| Vaccine | CATAGCCCATATATGGAGTTCCGCGTTACATAACTTACGGTAAATGGCCCGCCT | |
| #26 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-Two | GTAAACTGCCCACTTGGCAGTACATCAAGTGTATCATATGCCAAGTACGCCCCC | |
| Proline | TATTGACGTCAATGACGGTAAATGGCCCGCCTGGCATTATGCCCAGTACATGAC | |
| substi- | CTTATGGGACTTTCCTACTTGGCAGTACATCTACGTATTAGTCATCGCTATTACC | |
| tution | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGGGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCAGGTGG | |
| | GGGTGCCGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGTTCGTGTTCCTGGTGCTGCTGC | |
| | CCCTGGTGAGCAGCCAGTGCGTGAACCTGACCACCAGGACCCAGCTGCCCC | |
| | CCGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAGC | |
| | AACGTGACCTGGTTCCACGCCATCCACGTGAGCGGCACCAACGGCACCAAGA | |
| | GGTTCGACAACCCCGTGCTGCCCTTCAACGACGGCGTGTACTTCGCCAGCAC | |
| | CGAGAAGAGCAACATCATCAGGGGCTGGATCTTCGGCACCACCCTGGACAGC | |
| | AAGACCCAGAGCCTGCTGATCGTGAACAACGCCACCAACGTGGTGATCAAGG | |
| | TGTGCGAGTTCCAGTTCTGCAACGACCCCTTCCTGGGCGTGTACTACCACAAG | |
| | AACAACAAGAGCTGGATGGAGAGCGAGTTCAGGGTGTACAGCAGCGCCAACA | |
| | ACTGCACCTTCGAGTACGTGAGCCAGCCCTTCCTGATGGACCTGGAGGGCAA | |
| | GCAGGGCAACTTCAAGAACCTGAGGGAGTTCGTGTTCAAGAACATCGACGGC | |
| | TACTTCAAGATCTACAGCAAGCACACCCCCATCAACCTGGTGAGGGACCTGCC | |
| | CCAGGGCTTCAGCGCCCTGGAGCCCCTGGTGGACCTGCCCATCGGCATCAAC | |
| | ATCACCAGGTTCCAGACCCTGCTGGCCCTGCACAGGAGCTACCTGACCCCCG | |
| | GCGACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCAC | |
| | CGACGCCGTGGACTGCGCCCTGGACCCCCTGAGCGAGACCAAGTGCACCCT | |
| | GAAGAGCTTCACCGTGGAGAAGGGCATCTACCAGACCAGCAACTTCAGGGTG | |
| | CAGCCCACCGAGAGCATCGTGAGGTTCCCCAACATCACCAACCTGTGCCCCT | |
| | TCGGCGAGGTGTTCAACGCCACCAGGTTCGCCAGCGTGTACGCCTGGAACAG | |
| | GAAGAGGATCAGCAACTGCGTGGCCGACTACAGCGTGCTGTACAACAGCGCC | |
| | AGCTTCAGCACCTTCAAGTGCTACGGCGTGAGCCCCACCAAGCTGAACGACC | |
| | TGTGCTTCACCAACGTGTACGCCGACAGCTTCGTGATCAGGGGCGACGAGGT | |
| | GAGGCAGATCGCCCCCGGCCAGACCGGCAAGATCGCCGACTACAACTACAAG | |
| | CTGCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGAG | |
| | CAACCTGAAGCCCTTCGAGAGGGACATCAGCACCGAGATCTACCAGGCCGGC | |
| | AGCACCCCCTGCAACGGCGTGGAGGGCTTCAACTGCTACTTCCCCCTGCAGA | |
| | GCTACGGCTTCCAGCCCACCAACGGCGTGGGCTACCAGCCCTACAGGGTGGT | |
| | GGTGCTGAGCTTCGAGCTGCTGCACGCCCCCGCCACCGTGTGCGGCCCCAA | |
| | GAAGAGCACCAACCTGGTGAAGAACAAGTGCGTGAACTTCAACTTCAACGGC | |
| | CTGACCGGCACCGGCGTGCTGACCGAGAGCAACAAGAAGTTCCTGCCCTTCC | |
| | AGCAGTTCGGCAGGGACATCGCCGACACCACCGACGCCGTGAGGGACCCCC | |
| | AGACCCTGGAGATCCTGGACATCACCCCCTGCAGCTTCGGCGGCGTGAGCGT | |
| | GATCACCCCCGGCACCAACACCAGCAACCAGGTGGCCGTGCTGTACCAGGAC | |
| | GTGAACTGCACCGAGGTGCCCGTGGCCATCCACGCCGACCAGCTGACCCCC | |
| | ACCTGGAGGGTGTACAGCACCGGCAGCAACGTGTTCCAGACCAGGGCCGGC | |
| | TGCCTGATCGGCGCCGAGCACGTGAACAACAGCTACGAGTGCGACATCCCCA | |
| | TCGGCGCCGGCATCTGCGCCAGCTACCAGACCCAGACCAACAGCCCCAGGA | |
| | GGGCCAGGAGCGTGGCCAGCCAGAGCATCATCGCCTACACCATGAGCCTGGG | |
| | CGCCGAGAACAGCGTGGCCTACAGCAACAACAGCATCGCCATCCCCACCAAC | |
| | TTCACCATCAGCGTGACCACCGAGATCCTGCCCGTGAGCATGACCAAGACCA | |
| | GCGTGGACTGCACCATGTACATCTGCGGCGACAGCACCGAGTGCAGCAACCT | |
| | GCTGCTGCAGTACGGCAGCTTCTGCACCCAGCTGAACAGGGCCCTGACCGGC | |
| | ATCGCCGTGGAGCAGGACAAGAACACCCAGGAGGTGTTCGCCCAGGTGAAG | |
| | CAGATCTACAAGACCCCCCCCATCAAGGACTTCGGCGGCTTCAACTTCAGCCA | |
| | GATCCTGCCCGACCCCAGCAAGCCCAGCAAGAGGAGCTTCATCGAGGACCTG | |
| | CTGTTCAACAAGGTGACCCTGGCCGACGCCGGCTTCATCAAGCAGTACGGCG | |
| | ACTGCCTGGGCGACATCGCCGCCAGGGACCTGATCTGCGCCCAGAAGTTCAA | |
| | CGGCCTGACCGTGCTGCCCCCCCTGCTGACCGACGAGATGATCGCCCAGTAC | |
| | ACCAGCGCCCTGCTGGCCGGCACCATCACCAGCGGCTGGACCTTCGGCGCC | |
| | GGCGCCGCCCTGCAGATCCCCTTCGCCATGCAGATGGCCTACAGGTTCAACG | |
| | GCATCGGCGTGACCCAGAACGTGCTGTACGAGAACCAGAAGCTGATCGCCAA | |
| | CCAGTTCAACAGCGCCATCGGCAAGATCCAGGACAGCCTGAGCAGCACCGCC | |
| | AGCGCCCTGGGCAAGCTGCAGGACGTGGTGAACCAGAACGCCCAGGCCCTG | |
| | AACACCCTGGTGAAGCAGCTGAGCAGCAACTTCGGCGCCATCAGCAGCGTGC | |
| | TGAACGACATCCTGAGCAGGCTGGACCCCCCCGAGGCCGAGGTGCAGATCG | |
| | ACAGGCTGATCACCGGCAGGCTGCAGAGCCTGCAGACCTACGTGACCCAGCA | |
| | GCTGATCAGGGCCGCCGAGATCAGGGCCAGCGCCAACCTGGCCGCCACCAA | |
| | GATGAGCGAGTGCGTGCTGGGCCAGAGCAAGAGGGTGGACTTCTGCGGCAA | |
| | GGGCTACCACCTGATGAGCTTCCCCCAGAGCGCCCCCCACGGCGTGGTGTTC | |
| | CTGCACGTGACCTACGTGCCCGCCCAGGAGAAGAACTTCACCACCGCCCCCG | |
| | CCATCTGCCACGACGGCAAGGCCCACTTCCCCAGGGAGGGCGTGTTCGTGA | |
| | GCAACGGCACCCACTGGTTCGTGACCCAGAGGAACTTCTACGAGCCCCAGAT | |
| | CATCACCACCGACAACACCTTCGTGAGCGGCAACTGCGACGTGGTGATCGGC | |
| | ATCGTGAACAACACCGTGTACGACCCCCTGCAGCCCGAGCTGGACAGCTTCA | |
| | AGGAGGAGCTGGACAAGTACTTCAAGAACCACACCAGCCCCGACGTGGACCT | |
| | GGGCGACATCAGCGGCATCAACGCCAGCGTGGTGAACATCCAGAAGGAGATC | |
| | GACAGGCTGAACGAGGTGGCCAAGAACCTGAACGAGAGCCTGATCGACCTGC | |
| | AGGAGCTGGGCAAGTACGAGCAGTACATCAAGTGGCCCTGGTACATCTGGCT | |
| | GGGCTTCATCGCCGGCCTGATCGCCATCGTGATGGTGACCATCATGCTGTGCT | |
| | GCATGACCAGCTGCTGCAGCTGCCTGAAGGGCTGCTGCAGCTGCGGCAGCT | |
| | GCTGCAAGTTCGACGAGGACGACAGCGAGCCCGTGCTGAAGGGCGTGAAGC | |
| | TGCACTACACCTGATGACTCGAGCTGGTACTGCATGCACGCAATGCTAGCTGC | |
| | CCCTTTCCCGTCCTGGGTACCCCGAGTCTCCCCCGACCTCGGGTCCCAGGTA | |
| | TGCTCCCACCTCCACCTGCCCCACTCACCACCTCTGCTAGTTCCAGACACCTC | |
| | CCAAGCACGCAGCAATGCAGCTCAAAACGCTTAGCCTAGCCACACCCCCACG | |
| | GGAAACAGCAGTGATTAACCTTTAGCAATAAACGAAAGTTTAACTAAGCTATACT | |
| | AACCCCAGGGTTGGTCAATTTCGTGCCAGCCACACCCTGGAGCTAGCAAAAAA | |
| | AA | |
| Pan-CoV | TAGTTATTAATAGTAATCAATTACGGGGTCATTAGTTCATAGCCCATATATGGAGT | 90 |
| Vaccine | TCCGCGTTACATAACTTACGGTAAATGGCCCGCCTGGCTGACCGCCCAACGAC | |
| #27 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-Hexa | GTACATCAAGTGTATCATATGCCAAGTACGCCCCCTATTGACGTCAATGACGGT | |
| Pro | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCGGCAGCGCCAGCAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCCCCATCGAGGACCTGCTGTTCAACAAGGTGACCCTGGCCGACGCCG | |
| | GCTTCATCAAGCAGTACGGCGACTGCCTGGGCGACATCGCCGCCAGGGACCT | |
| | GATCTGCGCCCAGAAGTTCAACGGCCTGACCGTGCTGCCCCCCCTGCTGACC | |
| | GACGAGATGATCGCCCAGTACACCAGCGCCCTGCTGGCCGGCACCATCACCA | |
| | GCGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTACGA | |
| | GAACCAGAAGCTGATCGCCAACCAGTTCAACAGCGCCATCGGCAAGATCCAG | |
| | GACAGCCTGAGCAGCACCCCCAGCGCCCTGGGCAAGCTGCAGGACGTGGTG | |
| | AACCAGAACGCCCAGGCCCTGAACACCCTGGTGAAGCAGCTGAGCAGCAACT | |
| | TCGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCTGATGACTCGAGCTGGTAC | |
| | TGCATGCACGCAATGCTAGCTGCCCCTTTCCCGTCCTGGGTACCCCGAGTCTC | |
| | CCCCGACCTCGGGTCCCAGGTATGCTCCCACCTCCACCTGCCCCACTCACCA | |
| | CCTCTGCTAGTTCCAGACACCTCCCAAGCACGCAGCAATGCAGCTCAAAACGC | |
| | TTAGCCTAGCCACACCCCCACGGGAAACAGCAGTGATTAACCTTTAGCAATAAA | |
| | CGAAAGTTTAACTAAGCTATACTAACCCCAGGGTTGGTCAATTTCGTGCCAGCC | |
| | ACACCCTGGAGCTAGCAAAAAAAA | |
| Pan-CoV | TAGTTATTAATAGTAATCAATTACGGGGTCATTAGTTCATAGCCCATATATGGAGT | 91 |
| Vaccine | TCCGCGTTACATAACTTACGGTAAATGGCCCGCCTGGCTGACCGCCCAACGAC | |
| #28 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-Two | GTACATCAAGTGTATCATATGCCAAGTACGCCCCCTATTGACGTCAATGACGGT | |
| Proline | AAATGGCCCGCCTGGCATTATGCCCAGTACATGACCTTATGGGACTTTCCTACT | |
| substi- | TGGCAGTACATCTACGTATTAGTCATCGCTATTACCATGGTGATGCGGTTTTGGC | |
| tution | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TTCGTGTTCCTGGTGCTGCTGCCCCTGGTGAGCAGCCAGTGCGTGAACCTGA | |
| | CCACCAGGACCCAGCTGCCCCCCGCCTACACCAACAGCTTCACCAGGGGCGT | |
| | GTACTACCCCGACAAGGTGTTCAGGAGCAGCGTGCTGCACAGCACCCAGGAC | |
| | CTGTTCCTGCCCTTCTTCAGCAACGTGACCTGGTTCCACGCCATCCACGTGAG | |
| | CGGCACCAACGGCACCAAGAGGTTCGACAACCCCGTGCTGCCCTTCAACGAC | |
| | GGCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAACGC | |
| | CACCAACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGTTCAG | |
| | GGTGTACAGCAGCGCCAACAACTGCACCTTCGAGTACGTGAGCCAGCCCTTC | |
| | CTGATGGACCTGGAGGGCAAGCAGGGCAACTTCAAGAACCTGAGGGAGTTCG | |
| | TGTTCAAGAACATCGACGGCTACTTCAAGATCTACAGCAAGCACACCCCCATCA | |
| | ACCTGGTGAGGGACCTGCCCCAGGGCTTCAGCGCCCTGGAGCCCCTGGTGG | |
| | ACCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGCGC | |
| | CGCCGCCTACTACGTGGGCTACCTGCAGCCCAGGACCTTCCTGCTGAAGTAC | |
| | AACGAGAACGGCACCATCACCGACGCCGTGGACTGCGCCCTGGACCCCCTG | |
| | AGCGAGACCAAGTGCACCCTGAAGAGCTTCACCGTGGAGAAGGGCATCTACC | |
| | AGACCAGCAACTTCAGGGTGCAGCCCACCGAGAGCATCGTGAGGTTCCCCAA | |
| | CATCACCAACCTGTGCCCCTTCGGCGAGGTGTTCAACGCCACCAGGTTCGCC | |
| | AGCGTGTACGCCTGGAACAGGAAGAGGATCAGCAACTGCGTGGCCGACTACA | |
| | GCGTGCTGTACAACAGCGCCAGCTTCAGCACCTTCAAGTGCTACGGCGTGAG | |
| | CCCCACCAAGCTGAACGACCTGTGCTTCACCAACGTGTACGCCGACAGCTTC | |
| | GTGATCAGGGGCGACGAGGTGAGGCAGATCGCCCCCGGCCAGACCGGCAAG | |
| | ATCGCCGACTACAACTACAAGCTGCCCGACGACTTCACCGGCTGCGTGATCGC | |
| | CTGGAACAGCAACAACCTGGACAGCAAGGTGGGCGGCAACTACAACTACCTG | |
| | TACAGGCTGTTCAGGAAGAGCAACCTGAAGCCCTTCGAGAGGGACATCAGCA | |
| | CCGAGATCTACCAGGCCGGCAGCACCCCCTGCAACGGCGTGGAGGGCTTCA | |
| | ACTGCTACTTCCCCCTGCAGAGCTACGGCTTCCAGCCCACCAACGGCGTGGG | |
| | CTACCAGCCCTACAGGGTGGTGGTGCTGAGCTTCGAGCTGCTGCACGCCCCC | |
| | GCCACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACCAC | |
| | CGACGCCGTGAGGGACCCCCAGACCCTGGAGATCCTGGACATCACCCCCTGC | |
| | AGCTTCGGCGGCGTGAGCGTGATCACCCCCGGCACCAACACCAGCAACCAG | |
| | GTGGCCGTGCTGTACCAGGACGTGAACTGCACCGAGGTGCCCGTGGCCATCC | |
| | ACGCCGACCAGCTGACCCCCACCTGGAGGGTGTACAGCACCGGCAGCAACG | |
| | TGTTCCAGACCAGGGCCGGCTGCCTGATCGGCGCCGAGCACGTGAACAACA | |
| | GCTACGAGTGCGACATCCCCATCGGCGCCGGCATCTGCGCCAGCTACCAGAC | |
| | CCAGACCAACAGCCCCAGGAGGGCCAGGAGCGTGGCCAGCCAGAGCATCAT | |
| | CGCCTACACCATGAGCCTGGGCGCCGAGAACAGCGTGGCCTACAGCAACAAC | |
| | AGCATCGCCATCCCCACCAACTTCACCATCAGCGTGACCACCGAGATCCTGCC | |
| | CGTGAGCATGACCAAGACCAGCGTGGACTGCACCATGTACATCTGCGGCGAC | |
| | AGCACCGAGTGCAGCAACCTGCTGCTGCAGTACGGCAGCTTCTGCACCCAGC | |
| | TGAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACTTC | |
| | GGCGGCTTCAACTTCAGCCAGATCCTGCCCGACCCCAGCAAGCCCAGCAAGA | |
| | GGAGCTTCATCGAGGACCTGCTGTTCAACAAGGTGACCCTGGCCGACGCCGG | |
| | CTTCATCAAGCAGTACGGCGACTGCCTGGGCGACATCGCCGCCAGGGACCTG | |
| | ATCTGCGCCCAGAAGTTCAACGGCCTGACCGTGCTGCCCCCCCTGCTGACCG | |
| | ACGAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGCA | |
| | GATGGCCTACAGGTTCAACGGCATCGGCGTGACCCAGAACGTGCTGTACGAG | |
| | AACCAGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTGGTGA | |
| | ACCAGAACGCCCAGGCCCTGAACACCCTGGTGAAGCAGCTGAGCAGCAACTT | |
| | CGGCGCCATCAGCAGCGTGCTGAACGACATCCTGAGCAGGCTGGACCCCCC | |
| | CGAGGCCGAGGTGCAGATCGACAGGCTGATCACCGGCAGGCTGCAGAGCCT | |
| | GCAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGAGC | |
| | GCCCCCCACGGCGTGGTGTTCCTGCACGTGACCTACGTGCCCGCCCAGGAG | |
| | AAGAACTTCACCACCGCCCCCGCCATCTGCCACGACGGCAAGGCCCACTTCC | |
| | CCAGGGAGGGCGTGTTCGTGAGCAACGGCACCCACTGGTTCGTGACCCAGA | |
| | GGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCTGATGACTCGAGCTGGTAC | |
| | TGCATGCACGCAATGCTAGCTGCCCCTTTCCCGTCCTGGGTACCCCGAGTCTC | |
| | CCCCGACCTCGGGTCCCAGGTATGCTCCCACCTCCACCTGCCCCACTCACCA | |
| | CCTCTGCTAGTTCCAGACACCTCCCAAGCACGCAGCAATGCAGCTCAAAACGC | |
| | TTAGCCTAGCCACACCCCCACGGGAAACAGCAGTGATTAACCTTTAGCAATAAA | |
| | CGAAAGTTTAACTAAGCTATACTAACCCCAGGGTTGGTCAATTTCGTGCCAGCC | |
| | ACACCCTGGAGCTAGCAAAAAAAA | |
| Pan-CoV | TAGTTATTAATAGTAATCAATTACGGGGTCATTAGTTCATAGCCCATATATGGAGT | 92 |
| Vaccine | TCCGCGTTACATAACTTACGGTAAATGGCCCGCCTGGCTGACCGCCCAACGAC | |
| #29 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-Nucle | GTACATCAAGTGTATCATATGCCAAGTACGCCCCCTATTGACGTCAATGACGGT | |
| ocapsid | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | AGCGACAACGGCCCCCAGAACCAGAGGAACGCCCCCAGGATCACCTTCGGC | |
| | GGCCCCAGCGACAGCACCGGCAGCAACCAGAACGGCGAGAGGAGCGGCGC | |
| | CAGGAGCAAGCAGAGGAGGCCCCAGGGCCTGCCCAACAACACCGCCAGCTG | |
| | GTTCACCGCCCTGACCCAGCACGGCAAGGAGGACCTGAAGTTCCCCAGGGG | |
| | CCAGGGCGTGCCCATCAACACCAACAGCAGCCCCGACGACCAGATCGGCTAC | |
| | TACAGGAGGGCCACCAGGAGGATCAGGGGCGGCGACGGCAAGATGAAGGAC | |
| | CTGAGCCCCAGGTGGTACTTCTACTACCTGGGCACCGGCCCCGAGGCCGGCC | |
| | TGCCCTACGGCGCCAACAAGGACGGCATCATCTGGGTGGCCACCGAGGGCG | |
| | CCCTGAACACCCCCAAGGACCACATCGGCACCAGGAACCCCGCCAACAACGC | |
| | CGCCATCGTGCTGCAGCTGCCCCAGGGCACCACCCTGCCCAAGGGCTTCTAC | |
| | GCCGAGGGCAGCAGGGGCGGCAGCCAGGCCAGCAGCAGGAGCAGCAGCAG | |
| | GAGCAGGAACAGCAGCAGGAACAGCACCCCCGGCAGCAGCAGGGGCACCAG | |
| | CCCCGCCAGGATGGCCGGCAACGGCGGCGACGCCGCCCTGGCCCTGCTGCT | |
| | GCTGGACAGGCTGAACCAGCTGGAGAGCAAGATGAGCGGCAAGGGCCAGCA | |
| | GCAGCAGGGCCAGACCGTGACCAAGAAGAGCGCCGCCGAGGCCAGCAAGAA | |
| | GCCCAGGCAGAAGAGGACCGCCACCAAGGCCTACAACGTGACCCAGGCCTT | |
| | CGGCAGGAGGGGCCCCGAGCAGACCCAGGGCAACTTCGGCGACCAGGAGC | |
| | TGATCAGGCAGGGCACCGACTACAAGCACTGGCCCCAGATCGCCCAGTTCGC | |
| | CCCCAGCGCCAGCGCCTTCTTCGGCATGAGCAGGATCGGCATGGAGGTGACC | |
| | CCCAGCGGCACCTGGCTGACCTACACCGGCGCCATCAAGCTGGACGACAAG | |
| | GACCCCAACTTCAAGGACCAGGTGATCCTGCTGAACAAGCACATCGACGCCTA | |
| | CAAGACCTTCCCCCCCACCGAGCCCAAGAAGGACAAGAAGAAGAAGGCCGAC | |
| | GAGACCCAGGCCCTGCCCCAGAGGCAGAAGAAGCAGCAGACCGTGACCCTG | |
| | CTGCCCGCCGCCGACCTGGACGACTTCAGCAAGCAGCTGCAGCAGAGCATGA | |
| | GCAGCGCCGACAGCACCCAGGCCTGATGACTCGAGCTGGTACTGCATGCACG | |
| | CAATGCTAGCTGCCCCTTTCCCGTCCTGGGTACCCCGAGTCTCCCCCGACCTC | |
| | GGGTCCCAGGTATGCTCCCACCTCCACCTGCCCCACTCACCACCTCTGCTAGT | |
| | TCCAGACACCTCCCAAGCACGCAGCAATGCAGCTCAAAACGCTTAGCCTAGCC | |
| | ACACCCCCACGGGAAACAGCAGTGATTAACCTTTAGCAATAAACGAAAGTTTAA | |
| | CTAAGCTATACTAACCCCAGGGTTGGTCAATTTCGTGCCAGCCACACCCTGGA | |
| | GCTAGCAAAAAAAA | |
| Pan-CoV | TAGTTATTAATAGTAATCAATTACGGGGTCATTAGTTCATAGCCCATATATGGAGT | 93 |
| Vaccine | TCCGCGTTACATAACTTACGGTAAATGGCCCGCCTGGCTGACCGCCCAACGAC | |
| #30 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-ORF1 | GTACATCAAGTGTATCATATGCCAAGTACGCCCCCTATTGACGTCAATGACGGT | |
| a/b | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | GACCAGGAACCCCGCCTGGAGGAAGGCCGTGTTCATCAGCCCCTACAACAGC | |
| | CAGAACGCCGTGGCCAGCAAGATCCTGGGCCTGCCCACCCAGACCGTGGAC | |
| | AGCAGCCAGGGCAGCGAGTACGACTACGTGATCTTCACCCAGACCACCGAGA | |
| | CCGCCCACAGCTGCAACGTGAACAGGTTCAACGTGGCCATCACCAGGGCCAA | |
| | GGTGGGCATCCTGTGCATCATGAGCGACAGGGACCTGTACGACAAGCTGCAG | |
| | TTCACCAGCCTGGAGATCCCCAGGAGGAACGTGGCCACCCTGCAGGCCGAG | |
| | AACGTGACCGGCCTGTTCAAGGACTGCAGCAAGGTGATCACCGGCCTGCACC | |
| | CCACCCAGGCCCCCACCCACCTGAGCGTGGACACCAAGTTCAAGACCGAGG | |
| | GCCTGTGCGTGGACATCCCCGGCATCCCCAAGGACATGACCTACAGGAGGCT | |
| | GATCAGCATGATGGGCTTCAAGATGAACTACCAGGTGAACGGCTACCCCAACA | |
| | TGTTCATCACCAGGGAGGAGGCCATCAGGCACGTGAGGGCCTGGATCGGCTT | |
| | CGACGTGGAGGGCTGCCACGCCACCAGGGAGGCCGTGGGCACCAACCTGCC | |
| | CCTGCAGCTGGGCTTCAGCACCGGCGTGAACCTGGTGGCCGTGCCCACCGG | |
| | CTACGTGGACACCCCCAACAACACCGACTTCAGCAGGGTGAGCGCCAAGCCC | |
| | CCCCCCGGCGACCAGTTCAAGCACCTGATCCCCCTGATGTACAAGGGCCTGC | |
| | CCTGGAACGTGGTGAGGATCAAGATCGTGCAGATGCTGAGCGACACCCTGAA | |
| | GAACCTGAGCGACAGGGTGGTGTTCGTGCTGTGGGCCCACGGCTTCGAGCT | |
| | GACCAGCATGAAGTACTTCGTGAAGATCGGCCCCGAGAGGACCTGCTGCCTG | |
| | TGCGACAGGAGGGCCACCTGCTTCAGCACCGCCAGCGACACCTACGCCTGCT | |
| | GGCACCACAGCATCGGCTTCGACTACGTGTACAACCCCTTCATGATCGACGTG | |
| | CAGCAGTGGGGCTTCACCGGCAACCTGCAGAGCAACCACGACCTGTACTGCC | |
| | AGGTGCACGGCAACGCCCACGTGGCCAGCTGCGACGCCATCATGACCAGGT | |
| | GCCTGGCCGTGCACGAGTGCTTCGTGAAGAGGGTGGACTGGACCATCGAGTA | |
| | CCCCATCATCGGCGACGAGCTGAAGATCAACGCCGCCTGCAGGAAGGTGCAG | |
| | CACATGGTGGTGAAGGCCGCCCTGCTGGCCGACAAGTTCCCCGTGCTGCACG | |
| | ACATCGGCAACCCCAAGGCCATCAAGTGCGTGCCCCAGGCCGACGTGGAGTG | |
| | GAAGTTCTACGACGCCCAGCCCTGCAGCGACAAGGCCTACAAGATCGAGGAG | |
| | CTGTTCTACAGCTACGCCACCCACAGCGACAAGTTCACCGACGGCGTGTGCC | |
| | TGTTCTGGAACTGCAACGTGGACAGGTACCCCGCCAACAGCATCGTGTGCAG | |
| | GTTCGACACCAGGGTGCTGAGCAACCTGAACCTGCCCGGCTGCGACGGCGG | |
| | CAGCCTGTACGTGAACAAGCACGCCTTCCACACCCCCGCCTTCGACAAGAGC | |
| | GCCTTCGTGAACCTGAAGCAGCTGCCCTTCTTCTACTACAGCGACAGCCCCTG | |
| | CGAGAGCCACGGCAAGCAGGTGGTGAGCGACATCGACTACGTGCCCCTGAA | |
| | GAGCGCCACCTGCATCACCAGGTGCAACCTGGGCGGCGCCGTGTGCAGGCA | |
| | CCACGCCAACGAGTACAGGCTGTACCTGGACGCCTACAACATGATGATCAGCG | |
| | CCGGCTTCAGCCTGTGGGTGTACAAGCAGTTCGACACCTACAACCTGTGGAA | |
| | CACCTTCACCAGGCTGCAGAGCCTGGAGAACGTGGCCTTCAACGTGGTGAAC | |
| | AAGGGCCACTTCGACGGCCAGCAGGGCGAGGTGCCCGTGAGCATCATCAACA | |
| | ACACCGTGTACACCAAGGTGGACGGCGTGGACGTGGAGCTGTTCGAGAACAA | |
| | GACCACCCTGCCCGTGAACGTGGCCTTCGAGCTGTGGGCCAAGAGGAACATC | |
| | AAGCCCGTGCCCGAGGTGAAGATCCTGAACAACCTGGGCGTGGACATCGCCG | |
| | CCAACACCGTGATCTGGGACTACAAGAGGGACGCCCCCGCCCACATCAGCAC | |
| | CATCGGCGTGTGCAGCATGACCGACATCGCCAAGAAGCCCACCGAGACCATC | |
| | TGCGCCCCCCTGACCGTGTTCTTCGACGGCAGGGTGGACGGCCAGGTGGAC | |
| | CTGTTCAGGAACGCCAGGAACGGCGTGCTGATCACCGAGGGCAGCGTGAAG | |
| | GGCCTGCAGCCCAGCGTGGGCCCCAAGCAGGCCAGCCTGAACGGCGTGACC | |
| | CTGATCGGCGAGGCCGTGAAGACCCAGTTCAACTACTACAAGAAGGTGGACG | |
| | GCGTGGTGCAGCAGCTGCCCGAGACCTACTTCACCCAGAGCAGGAACCTGCA | |
| | GGAGTTCAAGCCCAGGAGCCAGATGGAGATCGACTTCCTGGAGCTGGCCATG | |
| | GACGAGTTCATCGAGAGGTACAAGCTGGAGGGCTACGCCTTCGAGCACATCG | |
| | TGTACGGCGACTTCAGCCACAGCCAGCTGGGCGGCCTGCACCTGCTGATCGG | |
| | CCTGGCCAAGAGGTTCAAGGAGAGCCCCTTCGAGCTGGAGGACTTCATCCCC | |
| | ATGGACAGCACCGTGAAGAACTACTTCATCACCGACGCCCAGACCGGCAGCA | |
| | GCAAGTGCGTGTGCAGCGTGATCGACCTGCTGCTGGACGACTTCGTGGAGAT | |
| | CATCAAGAGCCAGGACCTGAGCGTGGTGAGCAAGGTGGTGAAGGTGACCATC | |
| | GACTACACCGAGATCAGCTTCATGCTGTGGTGCAAGGACGGCCACGTGGAGA | |
| | CCTTCTACCCCAAGCTGCAGAGCAGCCAGGCCTGGCAGCCCGGCGTGGCCAT | |
| | GCCCAACCTGTACAAGATGCAGAGGATGCTGCTGGAGAAGTGCGACCTGCAG | |
| | AACTACGGCGACAGCGCCACCCTGCCCAAGGGCATCATGATGAACGTGGCCA | |
| | AGTACACCCAGCTGTGCCAGTACCTGAACACCCTGACCCTGGCCGTGCCCTA | |
| | CAACATGAGGGTGATCCACTTCGGCGCCGGCAGCGACAAGGGCGTGGCCCC | |
| | CGGCACCGCCGTGCTGAGGCAGTGGCTGCCCACCGGCACCCTGCTGGTGGA | |
| | CAGCGACCTGAACGACTTCGTGAGCGACGCCGACAGCACCCTGATCGGCGAC | |
| | TGCGCCACCGTGCACACCGCCAACAAGTGGGACCTGATCATCAGCGACATGT | |
| | ACGACCCCAAGACCAAGAACGTGACCAAGGAGAACGACAGCAAGGAGGGCTT | |
| | CTTCACCTACATCTGCGGCTTCATCCAGCAGAAGCTGGCCCTGGGCGGCAGC | |
| | GTGGCCATCAAGATCACCGAGCACAGCTGGAACGCCGACCTGTACAAGCTGA | |
| | TGGGCCACTTCGCCTGGTGGACCGCCTTCGTGACCAACGTGAACGCCAGCAG | |
| | CAGCGAGGCCTTCCTGATCGGCTGCAACTACCTGGGCAAGCCCAGGGAGCAG | |
| | ATCGACGGCTACGTGATGCACGCCAACTACATCTTCTGGAGGAACACCAACCC | |
| | CATCCAGCTGAGCAGCTACAGCCTGTTCGACATGAGCAAGTTCCCCCTGAAGC | |
| | TTGATGACTCGAGCTGGTACTGCATGCACGCAATGCTAGCTGCCCCTTTCCCG | |
| | TCCTGGGTACCCCGAGTCTCCCCCGACCTCGGGTCCCAGGTATGCTCCCACC | |
| | TCCACCTGCCCCACTCACCACCTCTGCTAGTTCCAGACACCTCCCAAGCACGC | |
| | AGCAATGCAGCTCAAAACGCTTAGCCTAGCCACACCCCCACGGGAAACAGCA | |
| | GTGATTAACCTTTAGCAATAAACGAAAGTTTAACTAAGCTATACTAACCCCAGGG | |
| | TTGGTCAATTTCGTGCCAGCCACACCCTGGAGCTAGCAAAAAAAA | |
| Pan-CoV | TAGTTATTAATAGTAATCAATTACGGGGTCATTAGTTCATAGCCCATATATGGAGT | 94 |
| Vaccine | TCCGCGTTACATAACTTACGGTAAATGGCCCGCCTGGCTGACCGCCCAACGAC | |
| #31 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV- | GTACATCAAGTGTATCATATGCCAAGTACGCCCCCTATTGACGTCAATGACGGT | |
| Envelope | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | TACAGCTTCGTGAGCGAGGAGACCGGCACCCTGATCGTGAACAGCGTGCTGC | |
| | TGTTCCTGGCCTTCGTGGTGTTCCTGCTGGTGACCCTGGCCATCCTGACCGC | |
| | CCTGAGGCTGTGCGCCTACTGCTGCAACATCGTGAACGTGAGCCTGGTGAAG | |
| | CCCAGCTTCTACGTGTACAGCAGGGTGAAGAACCTGAACAGCAGCAGGGTGC | |
| | CCGACCTGCTGGTGTGATGACTCGAGCTGGTACTGCATGCACGCAATGCTAGC | |
| | TGCCCCTTTCCCGTCCTGGGTACCCCGAGTCTCCCCCGACCTCGGGTCCCAG | |
| | GTATGCTCCCACCTCCACCTGCCCCACTCACCACCTCTGCTAGTTCCAGACAC | |
| | CTCCCAAGCACGCAGCAATGCAGCTCAAAACGCTTAGCCTAGCCACACCCCCA | |
| | CGGGAAACAGCAGTGATTAACCTTTAGCAATAAACGAAAGTTTAACTAAGCTATA | |
| | CTAACCCCAGGGTTGGTCAATTTCGTGCCAGCCACACCCTGGAGCTAGCAAAA | |
| | AAAA | |
| Pan-CoV | TAGTTATTAATAGTAATCAATTACGGGGTCATTAGTTCATAGCCCATATATGGAGT | 95 |
| Vaccine | TCCGCGTTACATAACTTACGGTAAATGGCCCGCCTGGCTGACCGCCCAACGAC | |
| #32 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV- | GTACATCAAGTGTATCATATGCCAAGTACGCCCCCTATTGACGTCAATGACGGT | |
| Membrane | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | GCCGACAGCAACGGCACCATCACCGTGGAGGAGCTGAAGAAGCTGCTGGAG | |
| | CAGTGGAACCTGGTGATCGGCTTCCTGTTCCTGACCTGGATCTGCCTGCTGCA | |
| | GTTCGCCTACGCCAACAGGAACAGGTTCCTGTACATCATCAAGCTGATCTTCCT | |
| | GTGGCTGCTGTGGCCCGTGACCCTGGCCTGCTTCGTGCTGGCCGCCGTGTAC | |
| | AGGATCAACTGGATCACCGGCGGCATCGCCATCGCCATGGCCTGCCTGGTGG | |
| | GCCTGATGTGGCTGAGCTACTTCATCGCCAGCTTCAGGCTGTTCGCCAGGAC | |
| | CAGGAGCATGTGGAGCTTCAACCCCGAGACCAACATCCTGCTGAACGTGCCC | |
| | CTGCACGGCACCATCCTGACCAGGCCCCTGCTGGAGAGCGAGCTGGTGATCG | |
| | GCGCCGTGATCCTGAGGGGCCACCTGAGGATCGCCGGCCACCACCTGGGCA | |
| | GGTGCGACATCAAGGACCTGCCCAAGGAGATCACCGTGGCCACCAGCAGGAC | |
| | CCTGAGCTACTACAAGCTGGGCGCCAGCCAGAGGGTGGCCGGCGACAGCGG | |
| | CTTCGCCGCCTACAGCAGGTACAGGATCGGCAACTACAAGCTGAACACCGAC | |
| | CACAGCAGCAGCAGCGACAACATCGCCCTGCTGGTGCAGTGATGACTCGAGC | |
| | TGGTACTGCATGCACGCAATGCTAGCTGCCCCTTTCCCGTCCTGGGTACCCCG | |
| | AGTCTCCCCCGACCTCGGGTCCCAGGTATGCTCCCACCTCCACCTGCCCCAC | |
| | TCACCACCTCTGCTAGTTCCAGACACCTCCCAAGCACGCAGCAATGCAGCTCA | |
| | AAACGCTTAGCCTAGCCACACCCCCACGGGAAACAGCAGTGATTAACCTTTAG | |
| | CAATAAACGAAAGTTTAACTAAGCTATACTAACCCCAGGGTTGGTCAATTTCGTG | |
| | CCAGCCACACCCTGGAGCTAGCAAAAAAAA | |
| Pan-CoV | TAGTTATTAATAGTAATCAATTACGGGGTCATTAGTTCATAGCCCATATATGGAGT | 96 |
| Vaccine | TCCGCGTTACATAACTTACGGTAAATGGCCCGCCTGGCTGACCGCCCAACGAC | |
| #33 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-ORF7 | GTACATCAAGTGTATCATATGCCAAGTACGCCCCCTATTGACGTCAATGACGGT | |
| a | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | AAGATCATCCTGTTCCTGGCCCTGATCACCCTGGCCACCTGCGAGCTGTACCA | |
| | CTACCAGGAGTGCGTGAGGGGCACCACCGTGTGATGACTCGAGCTGGTACTG | |
| | CATGCACGCAATGCTAGCTGCCCCTTTCCCGTCCTGGGTACCCCGAGTCTCCC | |
| | CCGACCTCGGGTCCCAGGTATGCTCCCACCTCCACCTGCCCCACTCACCACC | |
| | TCTGCTAGTTCCAGACACCTCCCAAGCACGCAGCAATGCAGCTCAAAACGCTT | |
| | AGCCTAGCCACACCCCCACGGGAAACAGCAGTGATTAACCTTTAGCAATAAAC | |
| | GAAAGTTTAACTAAGCTATACTAACCCCAGGGTTGGTCAATTTCGTGCCAGCCA | |
| | CACCCTGGAGCTAGCAAAAAAAA | |
| Pan-CoV | TAGTTATTAATAGTAATCAATTACGGGGTCATTAGTTCATAGCCCATATATGGAGT | 97 |
| Vaccine | TCCGCGTTACATAACTTACGGTAAATGGCCCGCCTGGCTGACCGCCCAACGAC | |
| #34 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-NSP3 | GTACATCAAGTGTATCATATGCCAAGTACGCCCCCTATTGACGTCAATGACGGT | |
| | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | GCCCCCACCAAGGTGACCTTCGGCGACGACACCGTGATCGAGGTGCAGGGC | |
| | TACAAGAGCGTGAACATCACCTTCGAGCTGGACGAGAGGATCGACAAGGTGC | |
| | TGAACGAGAAGTGCAGCGCCTACACCGTGGAGCTGGGCACCGAGGTGAACG | |
| | AGTTCGCCTGCGTGGTGGCCGACGCCGTGATCAAGACCCTGCAGCCCGTGA | |
| | GCGAGCTGCTGACCCCCCTGGGCATCGACCTGGACGAGTGGAGCATGGCCA | |
| | CCTACTACCTGTTCGACGAGAGCGGCGAGTTCAAGCTGGCCAGCCACATGTAC | |
| | TGCAGCTTCTACCCCCCCGACGAGGACGAGGAGGAGGGCGACTGCGAGGAG | |
| | GAGGAGTTCGAGCCCAGCACCCAGTACGAGTACGGCACCGAGGACGACTACC | |
| | AGGGCAAGCCCCTGGAGTTCGGCGCCACCAGCGCCGCCCTGCAGCCCGAGG | |
| | AGGAGCAGGAGGAGGACTGGCTGGACGACGACAGCCAGCAGACCGTGGGC | |
| | CAGCAGGACGGCAGCGAGGACAACCAGACCACCACCATCCAGACCATCGTGG | |
| | AGGTGCAGCCCCAGCTGGAGATGGAGCTGACCCCCGTGGTGCAGACCATCG | |
| | AGGTGAACAGCTTCAGCGGCTACCTGAAGCTGACCGACAACGTGTACATCAAG | |
| | AACGCCGACATCGTGGAGGAGGCCAAGAAGGTGAAGCCCACCGTGGTGGTG | |
| | AACGCCGCCAACGTGTACCTGAAGCACGGCGGCGGCGTGGCCGGCGCCCTG | |
| | AACAAGGCCACCAACAACGCCATGCAGGTGGAGAGCGACGACTACATCGCCA | |
| | CCAACGGCCCCCTGAAGGTGGGCGGCAGCTGCGTGCTGAGCGGCCACAACC | |
| | TGGCCAAGCACTGCCTGCACGTGGTGGGCCCCAACGTGAACAAGGGCGAGG | |
| | ACATCCAGCTGCTGAAGAGCGCCTACGAGAACTTCAACCAGCACGAGGTGCT | |
| | GCTGGCCCCCCTGCTGAGCGCCGGCATCTTCGGCGCCGACCCCATCCACAG | |
| | CCTGAGGGTGTGCGTGGACACCGTGAGGACCAACGTGTACCTGGCCGTGTTC | |
| | GACAAGAACCTGTACGACAAGCTGGTGAGCAGCTTCCTGGAGATGAAGAGCG | |
| | AGAAGCAGGTGGAGCAGAAGATCGCCGAGATCCCCAAGGAGGAGGTGAAGC | |
| | CCTTCATCACCGAGAGCAAGCCCAGCGTGGAGCAGAGGAAGCAGGACGACAA | |
| | GAAGATCAAGGCCTGCGTGGAGGAGGTGACCACCACCCTGGAGGAGACCAA | |
| | GTTCCTGACCGAGAACCTGCTGCTGTACATCGACATCAACGGCAACCTGCACC | |
| | CCGACAGCGCCACCCTGGTGAGCGACATCGACATCACCTTCCTGAAGAAGGA | |
| | CGCCCCCTACATCGTGGGCGACGTGGTGCAGGAGGGCGTGCTGACCGCCGT | |
| | GGTGATCCCCACCAAGAAGGCCGGCGGCACCACCGAGATGCTGGCCAAGGC | |
| | CCTGAGGAAGGTGCCCACCGACAACTACATCACCACCTACCCCGGCCAGGGC | |
| | CTGAACGGCTACACCGTGGAGGAGGCCAAGACCGTGCTGAAGAAGTGCAAGA | |
| | GCGCCTTCTACATCCTGCCCAGCATCATCAGCAACGAGAAGCAGGAGATCCTG | |
| | GGCACCGTGAGCTGGAACCTGAGGGAGATGCTGGCCCACGCCGAGGAGACC | |
| | AGGAAGCTGATGCCCGTGTGCGTGGAGACCAAGGCCATCGTGAGCACCATCC | |
| | AGAGGAAGTACAAGGGCATCAAGATCCAGGAGGGCGTGGTGGACTACGGCGC | |
| | CAGGTTCTACTTCTACACCAGCAAGACCACCGTGGCCAGCCTGATCAACACCC | |
| | TGAACGACCTGAACGAGACCCTGGTGACCATGCCCCTGGGCTACGTGACCCA | |
| | CGGCCTGAACCTGGAGGAGGCCGCCAGGTACATGAGGAGCCTGAAGGTGCC | |
| | CGCCACCGTGAGCGTGAGCAGCCCCGACGCCGTGACCGCCTACAACGGCTA | |
| | CCTGACCAGCAGCAGCAAGACCCCCGAGGAGCACTTCATCGAGACCATCAGC | |
| | CTGGCCGGCAGCTACAAGGACTGGAGCTACAGCGGCCAGAGCACCCAGCTG | |
| | GGCATCGAGTTCCTGAAGAGGGGCGACAAGAGCGTGTACTACACCAGCAACC | |
| | CCACCACCTTCCACCTGGACGGCGAGGTGATCACCTTCGACAACCTGAAGAC | |
| | CCTGCTGAGCCTGAGGGAGGTGAGGACCATCAAGGTGTTCACCACCGTGGAC | |
| | AACATCAACCTGCACTGATGACTCGAGCTGGTACTGCATGCACGCAATGCTAG | |
| | CTGCCCCTTTCCCGTCCTGGGTACCCCGAGTCTCCCCCGACCTCGGGTCCCA | |
| | GGTATGCTCCCACCTCCACCTGCCCCACTCACCACCTCTGCTAGTTCCAGACA | |
| | CCTCCCAAGCACGCAGCAATGCAGCTCAAAACGCTTAGCCTAGCCACACCCC | |
| | CACGGGAAACAGCAGTGATTAACCTTTAGCAATAAACGAAAGTTTAACTAAGCT | |
| | ATACTAACCCCAGGGTTGGTCAATTTCGTGCCAGCCACACCCTGGAGCTAGCA | |
| | AAAAAAA | |
| Pan-CoV | TAGTTATTAATAGTAATCAATTACGGGGTCATTAGTTCATAGCCCATATATGGAGT | 98 |
| Vaccine | TCCGCGTTACATAACTTACGGTAAATGGCCCGCCTGGCTGACCGCCCAACGAC | |
| #35 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-NSP5 | GTACATCAAGTGTATCATATGCCAAGTACGCCCCCTATTGACGTCAATGACGGT | |
| | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | AGCGGCTTCAGGAAGATGGCCTTCCCCAGCGGCAAGGTGGAGGGCTGCATG | |
| | GTGCAGGTGACCTGCGGCACCACCACCCTGAACGGCCTGTGGCTGGACGAC | |
| | GTGGTGTACTGCCCCAGGCACGTGATCTGCACCAGCGAGGACATGCTGAACC | |
| | CCAACTACGAGGACCTGCTGATCAGGAAGAGCAACCACAACTTCCTGGTGCA | |
| | GGCCGGCAACGTGCAGCTGAGGGTGATCGGCCACAGCATGCAGAACTGCGT | |
| | GCTGAAGCTGAAGGTGGACACCGCCAACCCCAAGACCCCCAAGTACAAGTTC | |
| | GTGAGGATCCAGCCCGGCCAGACCTTCAGCGTGCTGGCCTGCTACAACGGCA | |
| | GCCCCAGCGGCGTGTACCAGTGCGCCATGAGGCCCAACTTCACCATCAAGGG | |
| | CAGCTTCCTGAACGGCAGCTGCGGCAGCGTGGGCTTCAACATCGACTACGAC | |
| | TGCGTGAGCTTCTGCTACATGCACCACATGGAGCTGCCCACCGGCGTGCACG | |
| | CCGGCACCGACCTGGAGGGCAACTTCTACGGCCCCTTCGTGGACAGGCAGA | |
| | CCGCCCAGGCCGCCGGCACCGACACCACCATCACCGTGAACGTGCTGGCCT | |
| | GGCTGTACGCCGCCGTGATCAACGGCGACAGGTGGTTCCTGAACAGGTTCAC | |
| | CACCACCCTGAACGACTTCAACCTGGTGGCCATGAAGTACAACTACGAGCCCC | |
| | TGACCCAGGACCACGTGGACATCCTGGGCCCCCTGAGCGCCCAGACCGGCA | |
| | TCGCCGTGCTGGACATGTGCGCCAGCCTGAAGGAGCTGCTGCAGAACGGCAT | |
| | GAACGGCAGGACCATCCTGGGCAGCGCCCTGCTGGAGGACGAGTTCACCCC | |
| | CTTCGACGTGGTGAGGCAGTGCAGCGGCGTGACCTTCCAGTGATGACTCGAG | |
| | CTGGTACTGCATGCACGCAATGCTAGCTGCCCCTTTCCCGTCCTGGGTACCCC | |
| | GAGTCTCCCCCGACCTCGGGTCCCAGGTATGCTCCCACCTCCACCTGCCCCA | |
| | CTCACCACCTCTGCTAGTTCCAGACACCTCCCAAGCACGCAGCAATGCAGCTC | |
| | AAAACGCTTAGCCTAGCCACACCCCCACGGGAAACAGCAGTGATTAACCTTTA | |
| | GCAATAAACGAAAGTTTAACTAAGCTATACTAACCCCAGGGTTGGTCAATTTCGT | |
| | GCCAGCCACACCCTGGAGCTAGCAAAAAAAA | |
| Pan-CoV | TAGTTATTAATAGTAATCAATTACGGGGTCATTAGTTCATAGCCCATATATGGAGT | 99 |
| Vaccine | TCCGCGTTACATAACTTACGGTAAATGGCCCGCCTGGCTGACCGCCCAACGAC | |
| #36 | CCCCGCCCATTGACGTCAATAATGACGTATGTTCCCATAGTAACGCCAATAGGG | |
| rAdv5-C | ACTTTCCATTGACGTCAATGGGTGGAGTATTTACGGTAAACTGCCCACTTGGCA | |
| MV-NSP1 | GTACATCAAGTGTATCATATGCCAAGTACGCCCCCTATTGACGTCAATGACGGT | |
| 2 | AAATGGCCCGCCTGGCATTATGCCCAGTACATGACCTTATGGGACTTTCCTACT | |
| | TGGCAGTACATCTACGTATTAGTCATCGCTATTACCATGGTGATGCGGTTTTGGC | |
| | AGTACATCAATGGGCGTGGATAGCGGTTTGACTCACGGGGATTTCCAAGTCTC | |
| | CACCCCATTGACGTCAATGGGAGTTTGTTTTGGCACCAAAATCAACGGGACTT | |
| | TCCAAAATGTCGTAACAACTCCGCCCCATTGACGCAAATGGGCGGTAGGCGTG | |
| | TACGGTGGGAGGTCTATATAAGCAGAGCTGGTTTAGTGAACCGTCAGATCGAG | |
| | AATAAACTAGTATTCTTCTGGTCCCCACAGACTCAGAGAGAACCCGCCACCATG | |
| | AGCGCCGACGCCCAGAGCTTCCTGAACAGGGTGTGCGGCGTGAGCGCCGCC | |
| | AGGCTGACCCCCTGCGGCACCGGCACCAGCACCGACGTGGTGTACAGGGCC | |
| | TTCGACATCTACAACGACAAGGTGGCCGGCTTCGCCAAGTTCCTGAAGACCAA | |
| | CTGCTGCAGGTTCCAGGAGAAGGACGAGGACGACAACCTGATCGACAGCTAC | |
| | TTCGTGGTGAAGAGGCACACCTTCAGCAACTACCAGCACGAGGAGACCATCTA | |
| | CAACCTGCTGAAGGACTGCCCCGCCGTGGCCAAGCACGACTTCTTCAAGTTC | |
| | AGGATCGACGGCGACATGGTGCCCCACATCAGCAGGCAGAGGCTGACCAAGT | |
| | ACACCATGGCCGACCTGGTGTACGCCCTGAGGCACTTCGACGAGGGCAACTG | |
| | CGACACCCTGAAGGAGATCCTGGTGACCTACAACTGCTGCGACGACGACTAC | |
| | TTCAACAAGAAGGACTGGTACGACTTCGTGGAGAACCCCGACATCCTGAGGG | |
| | TGTACGCCAACCTGGGCGAGAGGGTGAGGCAGGCCCTGCTGAAGACCGTGC | |
| | AGTTCTGCGACGCCATGAGGAACGCCGGCATCGTGGGCGTGCTGACCCTGGA | |
| | CAACCAGGACCTGAACGGCAACTGGTACGACTTCGGCGACTTCATCCAGACC | |
| | ACCCCCGGCAGCGGCGTGCCCGTGGTGGACAGCTACTACAGCCTGCTGATGC | |
| | CCATCCTGACCCTGACCAGGGCCCTGACCGCCGAGAGCCACGTGGACACCG | |
| | ACCTGACCAAGCCCTACATCAAGTGGGACCTGCTGAAGTACGACTTCACCGAG | |
| | GAGAGGCTGAAGCTGTTCGACAGGTACTTCAAGTACTGGGACCAGACCTACCA | |
| | CCCCAACTGCGTGAACTGCCTGGACGACAGGTGCATCCTGCACTGCGCCAAC | |
| | TTCAACGTGCTGTTCAGCACCGTGTTCCCCCCCACCAGCTTCGGCCCCCTGG | |
| | TGAGGAAGATCTTCGTGGACGGCGTGCCCTTCGTGGTGAGCACCGGCTACCA | |
| | CTTCAGGGAGCTGGGCGTGGTGCACAACCAGGACGTGAACCTGCACAGCAG | |
| | CAGGCTGAGCTTCAAGGAGCTGCTGGTGTACGCCGCCGACCCCGCCATGCAC | |
| | GCCGCCAGCGGCAACCTGCTGCTGGACAAGAGGACCACCTGCTTCAGCGTG | |
| | GCCGCCCTGACCAACAACGTGGCCTTCCAGACCGTGAAGCCCGGCAACTTCA | |
| | ACAAGGACTTCTACGACTTCGCCGTGAGCAAGGGCTTCTTCAAGGAGGGCAG | |
| | CAGCGTGGAGCTGAAGCACTTCTTCTTCGCCCAGGACGGCAACGCCGCCATC | |
| | AGCGACTACGACTACTACAGGTACAACCTGCCCACCATGTGCGACATCAGGCA | |
| | GCTGCTGTTCGTGGTGGAGGTGGTGGACAAGTACTTCGACTGCTACGACGGC | |
| | GGCTGCATCAACGCCAACCAGGTGATCGTGAACAACCTGGACAAGAGCGCCG | |
| | GCTTCCCCTTCAACAAGTGGGGCAAGGCCAGGCTGTACTACGACAGCATGAG | |
| | CTACGAGGACCAGGACGCCCTGTTCGCCTACACCAAGAGGAACGTGATCCCC | |
| | ACCATCACCCAGATGAACCTGAAGTACGCCATCAGCGCCAAGAACAGGGCCA | |
| | GGACCGTGGCCGGCGTGAGCATCTGCAGCACCATGACCAACAGGCAGTTCCA | |
| | CCAGAAGCTGCTGAAGAGCATCGCCGCCACCAGGGGCGCCACCGTGGTGAT | |
| | CGGCACCAGCAAGTTCTACGGCGGCTGGCACAACATGCTGAAGACCGTGTAC | |
| | AGCGACGTGGAGAACCCCCACCTGATGGGCTGGGACTACCCCAAGTGCGACA | |
| | GGGCCATGCCCAACATGCTGAGGATCATGGCCAGCCTGGTGCTGGCCAGGAA | |
| | GCACACCACCTGCTGCAGCCTGAGCCACAGGTTCTACAGGCTGGCCAACGAG | |
| | TGCGCCCAGGTGCTGAGCGAGATGGTGATGTGCGGCGGCAGCCTGTACGTG | |
| | AAGCCCGGCGGCACCAGCAGCGGCGACGCCACCACCGCCTACGCCAACAGC | |
| | GTGTTCAACATCTGCCAGGCCGTGACCGCCAACGTGAACGCCCTGCTGAGCA | |
| | CCGACGGCAACAAGATCGCCGACAAGTACGTGAGGAACCTGCAGCACAGGCT | |
| | GTACGAGTGCCTGTACAGGAACAGGGACGTGGACACCGACTTCGTGAACGAG | |
| | TTCTACGCCTACCTGAGGAAGCACTTCAGCATGATGATCCTGAGCGACGACGC | |
| | CGTGGTGTGCTTCAACAGCACCTACGCCAGCCAGGGCCTGGTGGCCAGCATC | |
| | AAGAACTTCAAGAGCGTGCTGTACTACCAGAACAACGTGTTCATGAGCGAGGC | |
| | CAAGTGCTGGACCGAGACCGACCTGACCAAGGGCCCCCACGAGTTCTGCAG | |
| | CCAGCACACCATGCTGGTGAAGCAGGGCGACGACTACGTGTACCTGCCCTAC | |
| | CCCGACCCCAGCAGGATCCTGGGCGCCGGCTGCTTCGTGGACGACATCGTG | |
| | AAGACCGACGGCACCCTGATGATCGAGAGGTTCGTGAGCCTGGCCATCGACG | |
| | CCTACCCCCTGACCAAGCACCCCAACCAGGAGTACGCCGACGTGTTCCACCT | |
| | GTACCTGCAGTACATCAGGAAGCTGCACGACGAGCTGACCGGCCACATGCTG | |
| | GACATGTACAGCGTGATGCTGACCAACGACAACACCAGCAGGTACTGGGAGC | |
| | CCGAGTTCTACGAGGCCATGTACACCCCCCACACCGTGCTGCAGTGATGACTC | |
| | GAGCTGGTACTGCATGCACGCAATGCTAGCTGCCCCTTTCCCGTCCTGGGTAC | |
| | CCCGAGTCTCCCCCGACCTCGGGTCCCAGGTATGCTCCCACCTCCACCTGCC | |
| | CCACTCACCACCTCTGCTAGTTCCAGACACCTCCCAAGCACGCAGCAATGCAG | |
| | CTCAAAACGCTTAGCCTAGCCACACCCCCACGGGAAACAGCAGTGATTAACCT | |
| | TTAGCAATAAACGAAAGTTTAACTAAGCTATACTAACCCCAGGGTTGGTCAATTT | |
| | CGTGCCAGCCACACCCTGGAGCTAGCAAAAAAAA | |
| Pan-CoV | CTCGACATTGATTATTGACTAGTTATTAATAGTAATCAATTACGGGGTCATTAGTT | 100 |
| Vaccine | CATAGCCCATATATGGAGTTCCGCGTTACATAACTTACGGTAAATGGCCCGCCT | |
| #37 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG- | GTAAACTGCCCACTTGGCAGTACATCAAGTGTATCATATGCCAAGTACGCCCCC | |
| Nucleo- | TATTGACGTCAATGACGGTAAATGGCCCGCCTGGCATTATGCCCAGTACATGAC | |
| capsid | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGCGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGAGCGACAACGGCCCCCAGAACC | |
| | AGAGGAACGCCCCCAGGATCACCTTCGGCGGCCCCAGCGACAGCACCGGCA | |
| | GCAACCAGAACGGCGAGAGGAGCGGCGCCAGGAGCAAGCAGAGGAGGCCC | |
| | CAGGGCCTGCCCAACAACACCGCCAGCTGGTTCACCGCCCTGACCCAGCAC | |
| | GGCAAGGAGGACCTGAAGTTCCCCAGGGGCCAGGGCGTGCCCATCAACACC | |
| | AACAGCAGCCCCGACGACCAGATCGGCTACTACAGGAGGGCCACCAGGAGG | |
| | ATCAGGGGCGGCGACGGCAAGATGAAGGACCTGAGCCCCAGGTGGTACTTCT | |
| | ACTACCTGGGCACCGGCCCCGAGGCCGGCCTGCCCTACGGCGCCAACAAGG | |
| | ACGGCATCATCTGGGTGGCCACCGAGGGCGCCCTGAACACCCCCAAGGACC | |
| | ACATCGGCACCAGGAACCCCGCCAACAACGCCGCCATCGTGCTGCAGCTGCC | |
| | CCAGGGCACCACCCTGCCCAAGGGCTTCTACGCCGAGGGCAGCAGGGGCGG | |
| | CAGCCAGGCCAGCAGCAGGAGCAGCAGCAGGAGCAGGAACAGCAGCAGGAA | |
| | CAGCACCCCCGGCAGCAGCAGGGGCACCAGCCCCGCCAGGATGGCCGGCAA | |
| | CGGCGGCGACGCCGCCCTGGCCCTGCTGCTGCTGGACAGGCTGAACCAGCT | |
| | GGAGAGCAAGATGAGCGGCAAGGGCCAGCAGCAGCAGGGCCAGACCGTGAC | |
| | CAAGAAGAGCGCCGCCGAGGCCAGCAAGAAGCCCAGGCAGAAGAGGACCGC | |
| | CACCAAGGCCTACAACGTGACCCAGGCCTTCGGCAGGAGGGGCCCCGAGCA | |
| | GACCCAGGGCAACTTCGGCGACCAGGAGCTGATCAGGCAGGGCACCGACTA | |
| | CAAGCACTGGCCCCAGATCGCCCAGTTCGCCCCCAGCGCCAGCGCCTTCTTC | |
| | GGCATGAGCAGGATCGGCATGGAGGTGACCCCCAGCGGCACCTGGCTGACC | |
| | TACACCGGCGCCATCAAGCTGGACGACAAGGACCCCAACTTCAAGGACCAGG | |
| | TGATCCTGCTGAACAAGCACATCGACGCCTACAAGACCTTCCCCCCCACCGAG | |
| | CCCAAGAAGGACAAGAAGAAGAAGGCCGACGAGACCCAGGCCCTGCCCCAG | |
| | AGGCAGAAGAAGCAGCAGACCGTGACCCTGCTGCCCGCCGCCGACCTGGAC | |
| | GACTTCAGCAAGCAGCTGCAGCAGAGCATGAGCAGCGCCGACAGCACCCAG | |
| | GCCTGATGACTCGAGCTGGTACTGCATGCACGCAATGCTAGCTGCCCCTTTCC | |
| | CGTCCTGGGTACCCCGAGTCTCCCCCGACCTCGGGTCCCAGGTATGCTCCCA | |
| | CCTCCACCTGCCCCACTCACCACCTCTGCTAGTTCCAGACACCTCCCAAGCAC | |
| | GCAGCAATGCAGCTCAAAACGCTTAGCCTAGCCACACCCCCACGGGAAACAG | |
| | CAGTGATTAACCTTTAGCAATAAACGAAAGTTTAACTAAGCTATACTAACCCCAG | |
| | GGTTGGTCAATTTCGTGCCAGCCACACCCTGGAGCTAGCAAAAAAAA | |
| Pan-CoV | CTCGACATTGATTATTGACTAGTTATTAATAGTAATCAATTACGGGGTCATTAGTT | 101 |
| Vaccine | CATAGCCCATATATGGAGTTCCGCGTTACATAACTTACGGTAAATGGCCCGCCT | |
| #38 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-ORF1 | GTAAACTGCCCACTTGGCAGTACATCAAGTGTATCATATGCCAAGTACGCCCCC | |
| a/b | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGCGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGTGGCGGCAGGTGG | |
| | GGGTGCCGGGCGGGGCGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGGACCAGGAACCCCGCCTGGAG | |
| | GAAGGCCGTGTTCATCAGCCCCTACAACAGCCAGAACGCCGTGGCCAGCAAG | |
| | ATCCTGGGCCTGCCCACCCAGACCGTGGACAGCAGCCAGGGCAGCGAGTAC | |
| | GACTACGTGATCTTCACCCAGACCACCGAGACCGCCCACAGCTGCAACGTGA | |
| | ACAGGTTCAACGTGGCCATCACCAGGGCCAAGGTGGGCATCCTGTGCATCAT | |
| | GAGCGACAGGGACCTGTACGACAAGCTGCAGTTCACCAGCCTGGAGATCCCC | |
| | AGGAGGAACGTGGCCACCCTGCAGGCCGAGAACGTGACCGGCCTGTTCAAG | |
| | GACTGCAGCAAGGTGATCACCGGCCTGCACCCCACCCAGGCCCCCACCCAC | |
| | CTGAGCGTGGACACCAAGTTCAAGACCGAGGGCCTGTGCGTGGACATCCCCG | |
| | GCATCCCCAAGGACATGACCTACAGGAGGCTGATCAGCATGATGGGCTTCAAG | |
| | ATGAACTACCAGGTGAACGGCTACCCCAACATGTTCATCACCAGGGAGGAGGC | |
| | CATCAGGCACGTGAGGGCCTGGATCGGCTTCGACGTGGAGGGCTGCCACGC | |
| | CACCAGGGAGGCCGTGGGCACCAACCTGCCCCTGCAGCTGGGCTTCAGCAC | |
| | CGGCGTGAACCTGGTGGCCGTGCCCACCGGCTACGTGGACACCCCCAACAA | |
| | CACCGACTTCAGCAGGGTGAGCGCCAAGCCCCCCCCCGGCGACCAGTTCAA | |
| | GCACCTGATCCCCCTGATGTACAAGGGCCTGCCCTGGAACGTGGTGAGGATC | |
| | AAGATCGTGCAGATGCTGAGCGACACCCTGAAGAACCTGAGCGACAGGGTGG | |
| | TGTTCGTGCTGTGGGCCCACGGCTTCGAGCTGACCAGCATGAAGTACTTCGT | |
| | GAAGATCGGCCCCGAGAGGACCTGCTGCCTGTGCGACAGGAGGGCCACCTG | |
| | CTTCAGCACCGCCAGCGACACCTACGCCTGCTGGCACCACAGCATCGGCTTC | |
| | GACTACGTGTACAACCCCTTCATGATCGACGTGCAGCAGTGGGGCTTCACCGG | |
| | CAACCTGCAGAGCAACCACGACCTGTACTGCCAGGTGCACGGCAACGCCCAC | |
| | GTGGCCAGCTGCGACGCCATCATGACCAGGTGCCTGGCCGTGCACGAGTGCT | |
| | TCGTGAAGAGGGTGGACTGGACCATCGAGTACCCCATCATCGGCGACGAGCT | |
| | GAAGATCAACGCCGCCTGCAGGAAGGTGCAGCACATGGTGGTGAAGGCCGC | |
| | CCTGCTGGCCGACAAGTTCCCCGTGCTGCACGACATCGGCAACCCCAAGGCC | |
| | ATCAAGTGCGTGCCCCAGGCCGACGTGGAGTGGAAGTTCTACGACGCCCAGC | |
| | CCTGCAGCGACAAGGCCTACAAGATCGAGGAGCTGTTCTACAGCTACGCCAC | |
| | CCACAGCGACAAGTTCACCGACGGCGTGTGCCTGTTCTGGAACTGCAACGTG | |
| | GACAGGTACCCCGCCAACAGCATCGTGTGCAGGTTCGACACCAGGGTGCTGA | |
| | GCAACCTGAACCTGCCCGGCTGCGACGGCGGCAGCCTGTACGTGAACAAGC | |
| | ACGCCTTCCACACCCCCGCCTTCGACAAGAGCGCCTTCGTGAACCTGAAGCA | |
| | GCTGCCCTTCTTCTACTACAGCGACAGCCCCTGCGAGAGCCACGGCAAGCAG | |
| | GTGGTGAGCGACATCGACTACGTGCCCCTGAAGAGCGCCACCTGCATCACCA | |
| | GGTGCAACCTGGGCGGCGCCGTGTGCAGGCACCACGCCAACGAGTACAGGC | |
| | TGTACCTGGACGCCTACAACATGATGATCAGCGCCGGCTTCAGCCTGTGGGTG | |
| | TACAAGCAGTTCGACACCTACAACCTGTGGAACACCTTCACCAGGCTGCAGAG | |
| | CCTGGAGAACGTGGCCTTCAACGTGGTGAACAAGGGCCACTTCGACGGCCAG | |
| | CAGGGCGAGGTGCCCGTGAGCATCATCAACAACACCGTGTACACCAAGGTGG | |
| | ACGGCGTGGACGTGGAGCTGTTCGAGAACAAGACCACCCTGCCCGTGAACGT | |
| | GGCCTTCGAGCTGTGGGCCAAGAGGAACATCAAGCCCGTGCCCGAGGTGAA | |
| | GATCCTGAACAACCTGGGCGTGGACATCGCCGCCAACACCGTGATCTGGGAC | |
| | TACAAGAGGGACGCCCCCGCCCACATCAGCACCATCGGCGTGTGCAGCATGA | |
| | CCGACATCGCCAAGAAGCCCACCGAGACCATCTGCGCCCCCCTGACCGTGTT | |
| | CTTCGACGGCAGGGTGGACGGCCAGGTGGACCTGTTCAGGAACGCCAGGAA | |
| | CGGCGTGCTGATCACCGAGGGCAGCGTGAAGGGCCTGCAGCCCAGCGTGGG | |
| | CCCCAAGCAGGCCAGCCTGAACGGCGTGACCCTGATCGGCGAGGCCGTGAA | |
| | GACCCAGTTCAACTACTACAAGAAGGTGGACGGCGTGGTGCAGCAGCTGCCC | |
| | GAGACCTACTTCACCCAGAGCAGGAACCTGCAGGAGTTCAAGCCCAGGAGCC | |
| | AGATGGAGATCGACTTCCTGGAGCTGGCCATGGACGAGTTCATCGAGAGGTAC | |
| | AAGCTGGAGGGCTACGCCTTCGAGCACATCGTGTACGGCGACTTCAGCCACA | |
| | GCCAGCTGGGCGGCCTGCACCTGCTGATCGGCCTGGCCAAGAGGTTCAAGG | |
| | AGAGCCCCTTCGAGCTGGAGGACTTCATCCCCATGGACAGCACCGTGAAGAA | |
| | CTACTTCATCACCGACGCCCAGACCGGCAGCAGCAAGTGCGTGTGCAGCGTG | |
| | ATCGACCTGCTGCTGGACGACTTCGTGGAGATCATCAAGAGCCAGGACCTGA | |
| | GCGTGGTGAGCAAGGTGGTGAAGGTGACCATCGACTACACCGAGATCAGCTT | |
| | CATGCTGTGGTGCAAGGACGGCCACGTGGAGACCTTCTACCCCAAGCTGCAG | |
| | AGCAGCCAGGCCTGGCAGCCCGGCGTGGCCATGCCCAACCTGTACAAGATGC | |
| | AGAGGATGCTGCTGGAGAAGTGCGACCTGCAGAACTACGGCGACAGCGCCAC | |
| | CCTGCCCAAGGGCATCATGATGAACGTGGCCAAGTACACCCAGCTGTGCCAG | |
| | TACCTGAACACCCTGACCCTGGCCGTGCCCTACAACATGAGGGTGATCCACTT | |
| | CGGCGCCGGCAGCGACAAGGGCGTGGCCCCCGGCACCGCCGTGCTGAGGC | |
| | AGTGGCTGCCCACCGGCACCCTGCTGGTGGACAGCGACCTGAACGACTTCGT | |
| | GAGCGACGCCGACAGCACCCTGATCGGCGACTGCGCCACCGTGCACACCGC | |
| | CAACAAGTGGGACCTGATCATCAGCGACATGTACGACCCCAAGACCAAGAACG | |
| | TGACCAAGGAGAACGACAGCAAGGAGGGCTTCTTCACCTACATCTGCGGCTT | |
| | CATCCAGCAGAAGCTGGCCCTGGGCGGCAGCGTGGCCATCAAGATCACCGAG | |
| | CACAGCTGGAACGCCGACCTGTACAAGCTGATGGGCCACTTCGCCTGGTGGA | |
| | CCGCCTTCGTGACCAACGTGAACGCCAGCAGCAGCGAGGCCTTCCTGATCGG | |
| | CTGCAACTACCTGGGCAAGCCCAGGGAGCAGATCGACGGCTACGTGATGCAC | |
| | GCCAACTACATCTTCTGGAGGAACACCAACCCCATCCAGCTGAGCAGCTACAG | |
| | CCTGTTCGACATGAGCAAGTTCCCCCTGAAGCTTGATGACTCGAGCTGGTACT | |
| | GCATGCACGCAATGCTAGCTGCCCCTTTCCCGTCCTGGGTACCCCGAGTCTCC | |
| | CCCGACCTCGGGTCCCAGGTATGCTCCCACCTCCACCTGCCCCACTCACCAC | |
| | CTCTGCTAGTTCCAGACACCTCCCAAGCACGCAGCAATGCAGCTCAAAACGCT | |
| | TAGCCTAGCCACACCCCCACGGGAAACAGCAGTGATTAACCTTTAGCAATAAAC | |
| | GAAAGTTTAACTAAGCTATACTAACCCCAGGGTTGGTCAATTTCGTGCCAGCCA | |
| | CACCCTGGAGCTAGCAAAAAAAA | |
| Pan-CoV | CTCGACATTGATTATTGACTAGTTATTAATAGTAATCAATTACGGGGTCATTAGTT | 102 |
| Vaccine | CATAGCCCATATATGGAGTTCCGCGTTACATAACTTACGGTAAATGGCCCGCCT | |
| #39 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG- | GTAAACTGCCCACTTGGCAGTACATCAAGTGTATCATATGCCAAGTACGCCCCC | |
| Envelope | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGGGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCAGGTGG | |
| | GGGTGCCGGGCGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGTACAGCTTCGTGAGCGAGGAGA | |
| | CCGGCACCCTGATCGTGAACAGCGTGCTGCTGTTCCTGGCCTTCGTGGTGTT | |
| | CCTGCTGGTGACCCTGGCCATCCTGACCGCCCTGAGGCTGTGCGCCTACTGC | |
| | TGCAACATCGTGAACGTGAGCCTGGTGAAGCCCAGCTTCTACGTGTACAGCAG | |
| | GGTGAAGAACCTGAACAGCAGCAGGGTGCCCGACCTGCTGGTGTGATGACTC | |
| | GAGCTGGTACTGCATGCACGCAATGCTAGCTGCCCCTTTCCCGTCCTGGGTAC | |
| | CCCGAGTCTCCCCCGACCTCGGGTCCCAGGTATGCTCCCACCTCCACCTGCC | |
| | CCACTCACCACCTCTGCTAGTTCCAGACACCTCCCAAGCACGCAGCAATGCAG | |
| | CTCAAAACGCTTAGCCTAGCCACACCCCCACGGGAAACAGCAGTGATTAACCT | |
| | TTAGCAATAAACGAAAGTTTAACTAAGCTATACTAACCCCAGGGTTGGTCAATTT | |
| | CGTGCCAGCCACACCCTGGAGCTAGCAAAAAAAA | |
| Pan-CoV | CTCGACATTGATTATTGACTAGTTATTAATAGTAATCAATTACGGGGTCATTAGTT | 103 |
| Vaccine | CATAGCCCATATATGGAGTTCCGCGTTACATAACTTACGGTAAATGGCCCGCCT | |
| #40 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG- | GTAAACTGCCCACTTGGCAGTACATCAAGTGTATCATATGCCAAGTACGCCCCC | |
| Membrane | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGGGGGGGGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGGGGGGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGGCCGACAGCAACGGCACCATCA | |
| | CCGTGGAGGAGCTGAAGAAGCTGCTGGAGCAGTGGAACCTGGTGATCGGCTT | |
| | CCTGTTCCTGACCTGGATCTGCCTGCTGCAGTTCGCCTACGCCAACAGGAACA | |
| | GGTTCCTGTACATCATCAAGCTGATCTTCCTGTGGCTGCTGTGGCCCGTGACC | |
| | CTGGCCTGCTTCGTGCTGGCCGCCGTGTACAGGATCAACTGGATCACCGGCG | |
| | GCATCGCCATCGCCATGGCCTGCCTGGTGGGCCTGATGTGGCTGAGCTACTT | |
| | CATCGCCAGCTTCAGGCTGTTCGCCAGGACCAGGAGCATGTGGAGCTTCAAC | |
| | CCCGAGACCAACATCCTGCTGAACGTGCCCCTGCACGGCACCATCCTGACCA | |
| | GGCCCCTGCTGGAGAGCGAGCTGGTGATCGGCGCCGTGATCCTGAGGGGCC | |
| | ACCTGAGGATCGCCGGCCACCACCTGGGCAGGTGCGACATCAAGGACCTGC | |
| | CCAAGGAGATCACCGTGGCCACCAGCAGGACCCTGAGCTACTACAAGCTGGG | |
| | CGCCAGCCAGAGGGTGGCCGGCGACAGCGGCTTCGCCGCCTACAGCAGGTA | |
| | CAGGATCGGCAACTACAAGCTGAACACCGACCACAGCAGCAGCAGCGACAAC | |
| | ATCGCCCTGCTGGTGCAGTGATGACTCGAGCTGGTACTGCATGCACGCAATGC | |
| | TAGCTGCCCCTTTCCCGTCCTGGGTACCCCGAGTCTCCCCCGACCTCGGGTC | |
| | CCAGGTATGCTCCCACCTCCACCTGCCCCACTCACCACCTCTGCTAGTTCCAG | |
| | ACACCTCCCAAGCACGCAGCAATGCAGCTCAAAACGCTTAGCCTAGCCACACC | |
| | CCCACGGGAAACAGCAGTGATTAACCTTTAGCAATAAACGAAAGTTTAACTAAG | |
| | CTATACTAACCCCAGGGTTGGTCAATTTCGTGCCAGCCACACCCTGGAGCTAG | |
| | CAAAAAAAA | |
| Pan-CoV | CTCGACATTGATTATTGACTAGTTATTAATAGTAATCAATTACGGGGTCATTAGTT | 104 |
| Vaccine | CATAGCCCATATATGGAGTTCCGCGTTACATAACTTACGGTAAATGGCCCGCCT | |
| #41 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-ORF7 | GTAAACTGCCCACTTGGCAGTACATCAAGTGTATCATATGCCAAGTACGCCCCC | |
| a | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGGGGGGGGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGAAGATCATCCTGTTCCTGGCCCT | |
| | GATCACCCTGGCCACCTGCGAGCTGTACCACTACCAGGAGTGCGTGAGGGGC | |
| | ACCACCGTGTGATGACTCGAGCTGGTACTGCATGCACGCAATGCTAGCTGCCC | |
| | CTTTCCCGTCCTGGGTACCCCGAGTCTCCCCCGACCTCGGGTCCCAGGTATG | |
| | CTCCCACCTCCACCTGCCCCACTCACCACCTCTGCTAGTTCCAGACACCTCCC | |
| | AAGCACGCAGCAATGCAGCTCAAAACGCTTAGCCTAGCCACACCCCCACGGG | |
| | AAACAGCAGTGATTAACCTTTAGCAATAAACGAAAGTTTAACTAAGCTATACTAA | |
| | CCCCAGGGTTGGTCAATTTCGTGCCAGCCACACCCTGGAGCTAGCAAAAAAAA | |
| Pan-CoV | CTCGACATTGATTATTGACTAGTTATTAATAGTAATCAATTACGGGGTCATTAGTT | 105 |
| Vaccine | CATAGCCCATATATGGAGTTCCGCGTTACATAACTTACGGTAAATGGCCCGCCT | |
| #42 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-NSP3 | GTAAACTGCCCACTTGGCAGTACATCAAGTGTATCATATGCCAAGTACGCCCCC | |
| | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGCGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGGGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGGCCCCCACCAAGGTGACCTTCG | |
| | GCGACGACACCGTGATCGAGGTGCAGGGCTACAAGAGCGTGAACATCACCTT | |
| | CGAGCTGGACGAGAGGATCGACAAGGTGCTGAACGAGAAGTGCAGCGCCTAC | |
| | ACCGTGGAGCTGGGCACCGAGGTGAACGAGTTCGCCTGCGTGGTGGCCGAC | |
| | GCCGTGATCAAGACCCTGCAGCCCGTGAGCGAGCTGCTGACCCCCCTGGGC | |
| | ATCGACCTGGACGAGTGGAGCATGGCCACCTACTACCTGTTCGACGAGAGCG | |
| | GCGAGTTCAAGCTGGCCAGCCACATGTACTGCAGCTTCTACCCCCCCGACGA | |
| | GGACGAGGAGGAGGGCGACTGCGAGGAGGAGGAGTTCGAGCCCAGCACCC | |
| | AGTACGAGTACGGCACCGAGGACGACTACCAGGGCAAGCCCCTGGAGTTCGG | |
| | CGCCACCAGCGCCGCCCTGCAGCCCGAGGAGGAGCAGGAGGAGGACTGGC | |
| | TGGACGACGACAGCCAGCAGACCGTGGGCCAGCAGGACGGCAGCGAGGACA | |
| | ACCAGACCACCACCATCCAGACCATCGTGGAGGTGCAGCCCCAGCTGGAGAT | |
| | GGAGCTGACCCCCGTGGTGCAGACCATCGAGGTGAACAGCTTCAGCGGCTAC | |
| | CTGAAGCTGACCGACAACGTGTACATCAAGAACGCCGACATCGTGGAGGAGG | |
| | CCAAGAAGGTGAAGCCCACCGTGGTGGTGAACGCCGCCAACGTGTACCTGAA | |
| | GCACGGCGGCGGCGTGGCCGGCGCCCTGAACAAGGCCACCAACAACGCCAT | |
| | GCAGGTGGAGAGCGACGACTACATCGCCACCAACGGCCCCCTGAAGGTGGG | |
| | CGGCAGCTGCGTGCTGAGCGGCCACAACCTGGCCAAGCACTGCCTGCACGT | |
| | GGTGGGCCCCAACGTGAACAAGGGCGAGGACATCCAGCTGCTGAAGAGCGC | |
| | CTACGAGAACTTCAACCAGCACGAGGTGCTGCTGGCCCCCCTGCTGAGCGCC | |
| | GGCATCTTCGGCGCCGACCCCATCCACAGCCTGAGGGTGTGCGTGGACACC | |
| | GTGAGGACCAACGTGTACCTGGCCGTGTTCGACAAGAACCTGTACGACAAGC | |
| | TGGTGAGCAGCTTCCTGGAGATGAAGAGCGAGAAGCAGGTGGAGCAGAAGAT | |
| | CGCCGAGATCCCCAAGGAGGAGGTGAAGCCCTTCATCACCGAGAGCAAGCCC | |
| | AGCGTGGAGCAGAGGAAGCAGGACGACAAGAAGATCAAGGCCTGCGTGGAG | |
| | GAGGTGACCACCACCCTGGAGGAGACCAAGTTCCTGACCGAGAACCTGCTGC | |
| | TGTACATCGACATCAACGGCAACCTGCACCCCGACAGCGCCACCCTGGTGAG | |
| | CGACATCGACATCACCTTCCTGAAGAAGGACGCCCCCTACATCGTGGGCGAC | |
| | GTGGTGCAGGAGGGCGTGCTGACCGCCGTGGTGATCCCCACCAAGAAGGCC | |
| | GGCGGCACCACCGAGATGCTGGCCAAGGCCCTGAGGAAGGTGCCCACCGAC | |
| | AACTACATCACCACCTACCCCGGCCAGGGCCTGAACGGCTACACCGTGGAGG | |
| | AGGCCAAGACCGTGCTGAAGAAGTGCAAGAGCGCCTTCTACATCCTGCCCAG | |
| | CATCATCAGCAACGAGAAGCAGGAGATCCTGGGCACCGTGAGCTGGAACCTG | |
| | AGGGAGATGCTGGCCCACGCCGAGGAGACCAGGAAGCTGATGCCCGTGTGC | |
| | GTGGAGACCAAGGCCATCGTGAGCACCATCCAGAGGAAGTACAAGGGCATCA | |
| | AGATCCAGGAGGGCGTGGTGGACTACGGCGCCAGGTTCTACTTCTACACCAG | |
| | CAAGACCACCGTGGCCAGCCTGATCAACACCCTGAACGACCTGAACGAGACC | |
| | CTGGTGACCATGCCCCTGGGCTACGTGACCCACGGCCTGAACCTGGAGGAG | |
| | GCCGCCAGGTACATGAGGAGCCTGAAGGTGCCCGCCACCGTGAGCGTGAGC | |
| | AGCCCCGACGCCGTGACCGCCTACAACGGCTACCTGACCAGCAGCAGCAAGA | |
| | CCCCCGAGGAGCACTTCATCGAGACCATCAGCCTGGCCGGCAGCTACAAGGA | |
| | CTGGAGCTACAGCGGCCAGAGCACCCAGCTGGGCATCGAGTTCCTGAAGAGG | |
| | GGCGACAAGAGCGTGTACTACACCAGCAACCCCACCACCTTCCACCTGGACG | |
| | GCGAGGTGATCACCTTCGACAACCTGAAGACCCTGCTGAGCCTGAGGGAGGT | |
| | GAGGACCATCAAGGTGTTCACCACCGTGGACAACATCAACCTGCACTGATGAC | |
| | TCGAGCTGGTACTGCATGCACGCAATGCTAGCTGCCCCTTTCCCGTCCTGGGT | |
| | ACCCCGAGTCTCCCCCGACCTCGGGTCCCAGGTATGCTCCCACCTCCACCTG | |
| | CCCCACTCACCACCTCTGCTAGTTCCAGACACCTCCCAAGCACGCAGCAATGC | |
| | AGCTCAAAACGCTTAGCCTAGCCACACCCCCACGGGAAACAGCAGTGATTAAC | |
| | CTTTAGCAATAAACGAAAGTTTAACTAAGCTATACTAACCCCAGGGTTGGTCAAT | |
| | TTCGTGCCAGCCACACCCTGGAGCTAGCAAAAAAAA | |
| Pan-CoV | CTCGACATTGATTATTGACTAGTTATTAATAGTAATCAATTACGGGGTCATTAGTT | 106 |
| Vaccine | CATAGCCCATATATGGAGTTCCGCGTTACATAACTTACGGTAAATGGCCCGCCT | |
| #43 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-NSP5 | GTAAACTGCCCACTTGGCAGTACATCAAGTGTATCATATGCCAAGTACGCCCCC | |
| | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGGGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGAGCGGCTTCAGGAAGATGGCCT | |
| | TCCCCAGCGGCAAGGTGGAGGGCTGCATGGTGCAGGTGACCTGCGGCACCA | |
| | CCACCCTGAACGGCCTGTGGCTGGACGACGTGGTGTACTGCCCCAGGCACGT | |
| | GATCTGCACCAGCGAGGACATGCTGAACCCCAACTACGAGGACCTGCTGATCA | |
| | GGAAGAGCAACCACAACTTCCTGGTGCAGGCCGGCAACGTGCAGCTGAGGG | |
| | TGATCGGCCACAGCATGCAGAACTGCGTGCTGAAGCTGAAGGTGGACACCGC | |
| | CAACCCCAAGACCCCCAAGTACAAGTTCGTGAGGATCCAGCCCGGCCAGACC | |
| | TTCAGCGTGCTGGCCTGCTACAACGGCAGCCCCAGCGGCGTGTACCAGTGCG | |
| | CCATGAGGCCCAACTTCACCATCAAGGGCAGCTTCCTGAACGGCAGCTGCGG | |
| | CAGCGTGGGCTTCAACATCGACTACGACTGCGTGAGCTTCTGCTACATGCACC | |
| | ACATGGAGCTGCCCACCGGCGTGCACGCCGGCACCGACCTGGAGGGCAACT | |
| | TCTACGGCCCCTTCGTGGACAGGCAGACCGCCCAGGCCGCCGGCACCGACA | |
| | CCACCATCACCGTGAACGTGCTGGCCTGGCTGTACGCCGCCGTGATCAACGG | |
| | CGACAGGTGGTTCCTGAACAGGTTCACCACCACCCTGAACGACTTCAACCTG | |
| | GTGGCCATGAAGTACAACTACGAGCCCCTGACCCAGGACCACGTGGACATCC | |
| | TGGGCCCCCTGAGCGCCCAGACCGGCATCGCCGTGCTGGACATGTGCGCCA | |
| | GCCTGAAGGAGCTGCTGCAGAACGGCATGAACGGCAGGACCATCCTGGGCA | |
| | GCGCCCTGCTGGAGGACGAGTTCACCCCCTTCGACGTGGTGAGGCAGTGCA | |
| | GCGGCGTGACCTTCCAGTGATGACTCGAGCTGGTACTGCATGCACGCAATGCT | |
| | AGCTGCCCCTTTCCCGTCCTGGGTACCCCGAGTCTCCCCCGACCTCGGGTCC | |
| | CAGGTATGCTCCCACCTCCACCTGCCCCACTCACCACCTCTGCTAGTTCCAGA | |
| | CACCTCCCAAGCACGCAGCAATGCAGCTCAAAACGCTTAGCCTAGCCACACCC | |
| | CCACGGGAAACAGCAGTGATTAACCTTTAGCAATAAACGAAAGTTTAACTAAGC | |
| | TATACTAACCCCAGGGTTGGTCAATTTCGTGCCAGCCACACCCTGGAGCTAGC | |
| | AAAAAAAA | |
| Pan-CoV | CTCGACATTGATTATTGACTAGTTATTAATAGTAATCAATTACGGGGTCATTAGTT | 107 |
| Vaccine | CATAGCCCATATATGGAGTTCCGCGTTACATAACTTACGGTAAATGGCCCGCCT | |
| #44 | GGCTGACCGCCCAACGACCCCCGCCCATTGACGTCAATAATGACGTATGTTCC | |
| rAdv5-C | CATAGTAACGCCAATAGGGACTTTCCATTGACGTCAATGGGTGGAGTATTTACG | |
| AG-NSP1 | GTAAACTGCCCACTTGGCAGTACATCAAGTGTATCATATGCCAAGTACGCCCCC | |
| 2 | TATTGACGTCAATGACGGTAAATGGCCCGCCTGGCATTATGCCCAGTACATGAC | |
| | CTTATGGGACTTTCCTACTTGGCAGTACATCTACGTATTAGTCATCGCTATTACC | |
| | ATGGTCGAGGTGAGCCCCACGTTCTGCTTCACTCTCCCCATCTCCCCCCCCTC | |
| | CCCACCCCCAATTTTGTATTTATTTATTTTTTAATTATTTTGTGCAGCGATGGGGG | |
| | CGGGGGGGGGGGGGGGGCGCGCGCCAGGCGGGGCGGGGCGGGGCGAGG | |
| | GGCGGGGCGGGGCGAGGCGGAGAGGTGCGGCGGCAGCCAATCAGAGCGGC | |
| | GCGCTCCGAAAGTTTCCTTTTATGGCGAGGCGGCGGCGGCGGCGGCCCTATA | |
| | AAAAGCGAAGCGCGCGGCGGGCGGGAGTCGCTGCGCGCTGCCTTCGCCCC | |
| | GTGCCCCGCTCCGCCGCCGCCTCGCGCCGCCCGCCCCGGCTCTGACTGACC | |
| | GCGTTACTCCCACAGGTGAGCGGGCGGGACGGCCCTTCTCCTCCGGGCTGTA | |
| | ATTAGCGCTTGGTTTAATGACGGCTTGTTTCTTTTCTGTGGCTGCGTGAAAGCC | |
| | TTGAGGGGCTCCGGGAGGGCCCTTTGTGCGGGGGGAGCGGCTCGGGGGGT | |
| | GCGTGCGTGTGTGTGTGCGTGGGGAGCGCCGCGTGCGGCTCCGCGCTGCC | |
| | CGGCGGCTGTGAGCGCTGCGGGCGCGGCGCGGGGCTTTGTGCGCTCCGCA | |
| | GTGTGCGCGAGGGGAGCGCGGCCGGGGGCGGTGCCCCGCGGTGCGGGGG | |
| | GGGCTGCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGCACCCC | |
| | CCTCCCCGAGTTGCTGAGCACGGCCCGGCTTCGGGTGCGGGGCTCCGTACG | |
| | GGGCGTGGCGCGGGGCTCGCCGTGCCGGGGGGGGGGGGCGGCAGGTGG | |
| | GGGTGCCGGGGGGGGGGGGGCCGCCTCGGGCCGGGGAGGGCTCGGGGGA | |
| | GGGGCGCGGCGGCCCCCGGAGCGCCGGCGGCTGTCGAGGCGCGGCGAGC | |
| | CGCAGCCATTGCCTTTTATGGTAATCGTGCGAGAGGGCGCAGGGACTTCCTTT | |
| | GTCCCAAATCTGTGCGGAGCCGAAATCTGGGAGGCGCCGCCGCACCCCCTCT | |
| | AGCGGGCGCGGGGCGAAGCGGTGCGGCGCCGGCAGGAAGGAAATGGGCGG | |
| | GGAGGGCCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTTATTGT | |
| | GCTGTCTCATCATTTTGGCAAAGAATTGGAGAATAAACTAGTATTCTTCTGGTCC | |
| | CCACAGACTCAGAGAGAACCCGCCACCATGAGCGCCGACGCCCAGAGCTTCC | |
| | TGAACAGGGTGTGCGGCGTGAGCGCCGCCAGGCTGACCCCCTGCGGCACCG | |
| | GCACCAGCACCGACGTGGTGTACAGGGCCTTCGACATCTACAACGACAAGGT | |
| | GGCCGGCTTCGCCAAGTTCCTGAAGACCAACTGCTGCAGGTTCCAGGAGAAG | |
| | GACGAGGACGACAACCTGATCGACAGCTACTTCGTGGTGAAGAGGCACACCT | |
| | TCAGCAACTACCAGCACGAGGAGACCATCTACAACCTGCTGAAGGACTGCCC | |
| | CGCCGTGGCCAAGCACGACTTCTTCAAGTTCAGGATCGACGGCGACATGGTG | |
| | CCCCACATCAGCAGGCAGAGGCTGACCAAGTACACCATGGCCGACCTGGTGT | |
| | ACGCCCTGAGGCACTTCGACGAGGGCAACTGCGACACCCTGAAGGAGATCCT | |
| | GGTGACCTACAACTGCTGCGACGACGACTACTTCAACAAGAAGGACTGGTACG | |
| | ACTTCGTGGAGAACCCCGACATCCTGAGGGTGTACGCCAACCTGGGCGAGAG | |
| | GGTGAGGCAGGCCCTGCTGAAGACCGTGCAGTTCTGCGACGCCATGAGGAA | |
| | CGCCGGCATCGTGGGCGTGCTGACCCTGGACAACCAGGACCTGAACGGCAA | |
| | CTGGTACGACTTCGGCGACTTCATCCAGACCACCCCCGGCAGCGGCGTGCCC | |
| | GTGGTGGACAGCTACTACAGCCTGCTGATGCCCATCCTGACCCTGACCAGGG | |
| | CCCTGACCGCCGAGAGCCACGTGGACACCGACCTGACCAAGCCCTACATCAA | |
| | GTGGGACCTGCTGAAGTACGACTTCACCGAGGAGAGGCTGAAGCTGTTCGAC | |
| | AGGTACTTCAAGTACTGGGACCAGACCTACCACCCCAACTGCGTGAACTGCCT | |
| | GGACGACAGGTGCATCCTGCACTGCGCCAACTTCAACGTGCTGTTCAGCACC | |
| | GTGTTCCCCCCCACCAGCTTCGGCCCCCTGGTGAGGAAGATCTTCGTGGACG | |
| | GCGTGCCCTTCGTGGTGAGCACCGGCTACCACTTCAGGGAGCTGGGCGTGG | |
| | TGCACAACCAGGACGTGAACCTGCACAGCAGCAGGCTGAGCTTCAAGGAGCT | |
| | GCTGGTGTACGCCGCCGACCCCGCCATGCACGCCGCCAGCGGCAACCTGCT | |
| | GCTGGACAAGAGGACCACCTGCTTCAGCGTGGCCGCCCTGACCAACAACGTG | |
| | GCCTTCCAGACCGTGAAGCCCGGCAACTTCAACAAGGACTTCTACGACTTCGC | |
| | CGTGAGCAAGGGCTTCTTCAAGGAGGGCAGCAGCGTGGAGCTGAAGCACTTC | |
| | TTCTTCGCCCAGGACGGCAACGCCGCCATCAGCGACTACGACTACTACAGGTA | |
| | CAACCTGCCCACCATGTGCGACATCAGGCAGCTGCTGTTCGTGGTGGAGGTG | |
| | GTGGACAAGTACTTCGACTGCTACGACGGCGGCTGCATCAACGCCAACCAGG | |
| | TGATCGTGAACAACCTGGACAAGAGCGCCGGCTTCCCCTTCAACAAGTGGGG | |
| | CAAGGCCAGGCTGTACTACGACAGCATGAGCTACGAGGACCAGGACGCCCTG | |
| | TTCGCCTACACCAAGAGGAACGTGATCCCCACCATCACCCAGATGAACCTGAA | |
| | GTACGCCATCAGCGCCAAGAACAGGGCCAGGACCGTGGCCGGCGTGAGCAT | |
| | CTGCAGCACCATGACCAACAGGCAGTTCCACCAGAAGCTGCTGAAGAGCATC | |
| | GCCGCCACCAGGGGCGCCACCGTGGTGATCGGCACCAGCAAGTTCTACGGC | |
| | GGCTGGCACAACATGCTGAAGACCGTGTACAGCGACGTGGAGAACCCCCACC | |
| | TGATGGGCTGGGACTACCCCAAGTGCGACAGGGCCATGCCCAACATGCTGAG | |
| | GATCATGGCCAGCCTGGTGCTGGCCAGGAAGCACACCACCTGCTGCAGCCTG | |
| | AGCCACAGGTTCTACAGGCTGGCCAACGAGTGCGCCCAGGTGCTGAGCGAG | |
| | ATGGTGATGTGCGGCGGCAGCCTGTACGTGAAGCCCGGCGGCACCAGCAGC | |
| | GGCGACGCCACCACCGCCTACGCCAACAGCGTGTTCAACATCTGCCAGGCCG | |
| | TGACCGCCAACGTGAACGCCCTGCTGAGCACCGACGGCAACAAGATCGCCGA | |
| | CAAGTACGTGAGGAACCTGCAGCACAGGCTGTACGAGTGCCTGTACAGGAAC | |
| | AGGGACGTGGACACCGACTTCGTGAACGAGTTCTACGCCTACCTGAGGAAGC | |
| | ACTTCAGCATGATGATCCTGAGCGACGACGCCGTGGTGTGCTTCAACAGCACC | |
| | TACGCCAGCCAGGGCCTGGTGGCCAGCATCAAGAACTTCAAGAGCGTGCTGT | |
| | ACTACCAGAACAACGTGTTCATGAGCGAGGCCAAGTGCTGGACCGAGACCGA | |
| | CCTGACCAAGGGCCCCCACGAGTTCTGCAGCCAGCACACCATGCTGGTGAAG | |
| | CAGGGCGACGACTACGTGTACCTGCCCTACCCCGACCCCAGCAGGATCCTGG | |
| | GCGCCGGCTGCTTCGTGGACGACATCGTGAAGACCGACGGCACCCTGATGAT | |
| | CGAGAGGTTCGTGAGCCTGGCCATCGACGCCTACCCCCTGACCAAGCACCCC | |
| | AACCAGGAGTACGCCGACGTGTTCCACCTGTACCTGCAGTACATCAGGAAGCT | |
| | GCACGACGAGCTGACCGGCCACATGCTGGACATGTACAGCGTGATGCTGACC | |
| | AACGACAACACCAGCAGGTACTGGGAGCCCGAGTTCTACGAGGCCATGTACA | |
| | CCCCCCACACCGTGCTGCAGTGATGACTCGAGCTGGTACTGCATGCACGCAAT | |
| | GCTAGCTGCCCCTTTCCCGTCCTGGGTACCCCGAGTCTCCCCCGACCTCGGG | |
| | TCCCAGGTATGCTCCCACCTCCACCTGCCCCACTCACCACCTCTGCTAGTTCC | |
| | AGACACCTCCCAAGCACGCAGCAATGCAGCTCAAAACGCTTAGCCTAGCCACA | |
| | CCCCCACGGGAAACAGCAGTGATTAACCTTTAGCAATAAACGAAAGTTTAACTA | |
| | AGCTATACTAACCCCAGGGTTGGTCAATTTCGTGCCAGCCACACCCTGGAGCT | |
| | AGCAAAAAAAA | |
| Pan-CoV | ATGTTCGTGTTCCTGGTGCTGCTGCCCCTGGTGAGCAGCCAGTGCGTGAACC | 108 |
| Vaccine | TGACCACCAGGACCCAGCTGCCCCCCGCCTACACCAACAGCTTCACCAGGGG | |
| #45 (B1) | CGTGTACTACCCCGACAAGGTGTTCAGGAGCAGCGTGCTGCACAGCACCCAG | |
| | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGAG | |
| | CGACAACGGCCCCCAGAACCAGAGGAACGCCCCCAGGATCACCTTCGGCGG | |
| | CCCCAGCGACAGCACCGGCAGCAACCAGAACGGCGAGAGGAGCGGCGCCA | |
| | GGAGCAAGCAGAGGAGGCCCCAGGGCCTGCCCAACAACACCGCCAGCTGGT | |
| | TCACCGCCCTGACCCAGCACGGCAAGGAGGACCTGAAGTTCCCCAGGGGCC | |
| | AGGGCGTGCCCATCAACACCAACAGCAGCCCCGACGACCAGATCGGCTACTA | |
| | CAGGAGGGCCACCAGGAGGATCAGGGGCGGCGACGGCAAGATGAAGGACCT | |
| | GAGCCCCAGGTGGTACTTCTACTACCTGGGCACCGGCCCCGAGGCCGGCCT | |
| | GCCCTACGGCGCCAACAAGGACGGCATCATCTGGGTGGCCACCGAGGGCGC | |
| | CCTGAACACCCCCAAGGACCACATCGGCACCAGGAACCCCGCCAACAACGCC | |
| | GCCATCGTGCTGCAGCTGCCCCAGGGCACCACCCTGCCCAAGGGCTTCTACG | |
| | CCGAGGGCAGCAGGGGCGGCAGCCAGGCCAGCAGCAGGAGCAGCAGCAGG | |
| | AGCAGGAACAGCAGCAGGAACAGCACCCCCGGCAGCAGCAGGGGCACCAGC | |
| | CCCGCCAGGATGGCCGGCAACGGCGGCGACGCCGCCCTGGCCCTGCTGCTG | |
| | CTGGACAGGCTGAACCAGCTGGAGAGCAAGATGAGCGGCAAGGGCCAGCAG | |
| | CAGCAGGGCCAGACCGTGACCAAGAAGAGCGCCGCCGAGGCCAGCAAGAAG | |
| | CCCAGGCAGAAGAGGACCGCCACCAAGGCCTACAACGTGACCCAGGCCTTC | |
| | GGCAGGAGGGGCCCCGAGCAGACCCAGGGCAACTTCGGCGACCAGGAGCT | |
| | GATCAGGCAGGGCACCGACTACAAGCACTGGCCCCAGATCGCCCAGTTCGCC | |
| | CCCAGCGCCAGCGCCTTCTTCGGCATGAGCAGGATCGGCATGGAGGTGACCC | |
| | CCAGCGGCACCTGGCTGACCTACACCGGCGCCATCAAGCTGGACGACAAGGA | |
| | CCCCAACTTCAAGGACCAGGTGATCCTGCTGAACAAGCACATCGACGCCTACA | |
| | AGACCTTCCCCCCCACCGAGCCCAAGAAGGACAAGAAGAAGAAGGCCGACG | |
| | AGACCCAGGCCCTGCCCCAGAGGCAGAAGAAGCAGCAGACCGTGACCCTGC | |
| | TGCCCGCCGCCGACCTGGACGACTTCAGCAAGCAGCTGCAGCAGAGCATGA | |
| | GCAGCGCCGACAGCACCCAGGCCTGATGA | |
| Pan-CoV | ATGTTCGTGTTCCTGGTGCTGCTGCCCCTGGTGAGCAGCCAGTGCGTGAACC | 109 |
| Vaccine | TGACCACCAGGACCCAGCTGCCCCCCGCCTACACCAACAGCTTCACCAGGGG | |
| #46 (B2) | CGTGTACTACCCCGACAAGGTGTTCAGGAGCAGCGTGCTGCACAGCACCCAG | |
| | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCTGATGA | |
| Pan-CoV | ATGTTCGTGTTCCTGGTGCTGCTGCCCCTGGTGAGCAGCCAGTGCGTGAACC | 110 |
| Vaccine | TGACCACCAGGACCCAGCTGCCCCCCGCCTACACCAACAGCTTCACCAGGGG | |
| #47 (B4) | CGTGTACTACCCCGACAAGGTGTTCAGGAGCAGCGTGCTGCACAGCACCCAG | |
| | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGACCA | |
| | GGAACCCCGCCTGGAGGAAGGCCGTGTTCATCAGCCCCTACAACAGCCAGAA | |
| | CGCCGTGGCCAGCAAGATCCTGGGCCTGCCCACCCAGACCGTGGACAGCAG | |
| | CCAGGGCAGCGAGTACGACTACGTGATCTTCACCCAGACCACCGAGACCGCC | |
| | CACAGCTGCAACGTGAACAGGTTCAACGTGGCCATCACCAGGGCCAAGGTGG | |
| | GCATCCTGTGCATCATGAGCGACAGGGACCTGTACGACAAGCTGCAGTTCACC | |
| | AGCCTGGAGATCCCCAGGAGGAACGTGGCCACCCTGCAGGCCGAGAACGTG | |
| | ACCGGCCTGTTCAAGGACTGCAGCAAGGTGATCACCGGCCTGCACCCCACCC | |
| | AGGCCCCCACCCACCTGAGCGTGGACACCAAGTTCAAGACCGAGGGCCTGT | |
| | GCGTGGACATCCCCGGCATCCCCAAGGACATGACCTACAGGAGGCTGATCAG | |
| | CATGATGGGCTTCAAGATGAACTACCAGGTGAACGGCTACCCCAACATGTTCAT | |
| | CACCAGGGAGGAGGCCATCAGGCACGTGAGGGCCTGGATCGGCTTCGACGT | |
| | GGAGGGCTGCCACGCCACCAGGGAGGCCGTGGGCACCAACCTGCCCCTGCA | |
| | GCTGGGCTTCAGCACCGGCGTGAACCTGGTGGCCGTGCCCACCGGCTACGT | |
| | GGACACCCCCAACAACACCGACTTCAGCAGGGTGAGCGCCAAGCCCCCCCC | |
| | CGGCGACCAGTTCAAGCACCTGATCCCCCTGATGTACAAGGGCCTGCCCTGG | |
| | AACGTGGTGAGGATCAAGATCGTGCAGATGCTGAGCGACACCCTGAAGAACC | |
| | TGAGCGACAGGGTGGTGTTCGTGCTGTGGGCCCACGGCTTCGAGCTGACCA | |
| | GCATGAAGTACTTCGTGAAGATCGGCCCCGAGAGGACCTGCTGCCTGTGCGA | |
| | CAGGAGGGCCACCTGCTTCAGCACCGCCAGCGACACCTACGCCTGCTGGCA | |
| | CCACAGCATCGGCTTCGACTACGTGTACAACCCCTTCATGATCGACGTGCAGC | |
| | AGTGGGGCTTCACCGGCAACCTGCAGAGCAACCACGACCTGTACTGCCAGGT | |
| | GCACGGCAACGCCCACGTGGCCAGCTGCGACGCCATCATGACCAGGTGCCT | |
| | GGCCGTGCACGAGTGCTTCGTGAAGAGGGTGGACTGGACCATCGAGTACCCC | |
| | ATCATCGGCGACGAGCTGAAGATCAACGCCGCCTGCAGGAAGGTGCAGCACA | |
| | TGGTGGTGAAGGCCGCCCTGCTGGCCGACAAGTTCCCCGTGCTGCACGACAT | |
| | CGGCAACCCCAAGGCCATCAAGTGCGTGCCCCAGGCCGACGTGGAGTGGAA | |
| | GTTCTACGACGCCCAGCCCTGCAGCGACAAGGCCTACAAGATCGAGGAGCTG | |
| | TTCTACAGCTACGCCACCCACAGCGACAAGTTCACCGACGGCGTGTGCCTGTT | |
| | CTGGAACTGCAACGTGGACAGGTACCCCGCCAACAGCATCGTGTGCAGGTTC | |
| | GACACCAGGGTGCTGAGCAACCTGAACCTGCCCGGCTGCGACGGCGGCAGC | |
| | CTGTACGTGAACAAGCACGCCTTCCACACCCCCGCCTTCGACAAGAGCGCCT | |
| | TCGTGAACCTGAAGCAGCTGCCCTTCTTCTACTACAGCGACAGCCCCTGCGAG | |
| | AGCCACGGCAAGCAGGTGGTGAGCGACATCGACTACGTGCCCCTGAAGAGC | |
| | GCCACCTGCATCACCAGGTGCAACCTGGGCGGCGCCGTGTGCAGGCACCAC | |
| | GCCAACGAGTACAGGCTGTACCTGGACGCCTACAACATGATGATCAGCGCCG | |
| | GCTTCAGCCTGTGGGTGTACAAGCAGTTCGACACCTACAACCTGTGGAACACC | |
| | TTCACCAGGCTGCAGAGCCTGGAGAACGTGGCCTTCAACGTGGTGAACAAGG | |
| | GCCACTTCGACGGCCAGCAGGGCGAGGTGCCCGTGAGCATCATCAACAACAC | |
| | CGTGTACACCAAGGTGGACGGCGTGGACGTGGAGCTGTTCGAGAACAAGACC | |
| | ACCCTGCCCGTGAACGTGGCCTTCGAGCTGTGGGCCAAGAGGAACATCAAGC | |
| | CCGTGCCCGAGGTGAAGATCCTGAACAACCTGGGCGTGGACATCGCCGCCAA | |
| | CACCGTGATCTGGGACTACAAGAGGGACGCCCCCGCCCACATCAGCACCATC | |
| | GGCGTGTGCAGCATGACCGACATCGCCAAGAAGCCCACCGAGACCATCTGCG | |
| | CCCCCCTGACCGTGTTCTTCGACGGCAGGGTGGACGGCCAGGTGGACCTGT | |
| | TCAGGAACGCCAGGAACGGCGTGCTGATCACCGAGGGCAGCGTGAAGGGCC | |
| | TGCAGCCCAGCGTGGGCCCCAAGCAGGCCAGCCTGAACGGCGTGACCCTGA | |
| | TCGGCGAGGCCGTGAAGACCCAGTTCAACTACTACAAGAAGGTGGACGGCGT | |
| | GGTGCAGCAGCTGCCCGAGACCTACTTCACCCAGAGCAGGAACCTGCAGGAG | |
| | TTCAAGCCCAGGAGCCAGATGGAGATCGACTTCCTGGAGCTGGCCATGGACG | |
| | AGTTCATCGAGAGGTACAAGCTGGAGGGCTACGCCTTCGAGCACATCGTGTAC | |
| | GGCGACTTCAGCCACAGCCAGCTGGGCGGCCTGCACCTGCTGATCGGCCTG | |
| | GCCAAGAGGTTCAAGGAGAGCCCCTTCGAGCTGGAGGACTTCATCCCCATGG | |
| | ACAGCACCGTGAAGAACTACTTCATCACCGACGCCCAGACCGGCAGCAGCAA | |
| | GTGCGTGTGCAGCGTGATCGACCTGCTGCTGGACGACTTCGTGGAGATCATC | |
| | AAGAGCCAGGACCTGAGCGTGGTGAGCAAGGTGGTGAAGGTGACCATCGACT | |
| | ACACCGAGATCAGCTTCATGCTGTGGTGCAAGGACGGCCACGTGGAGACCTT | |
| | CTACCCCAAGCTGCAGAGCAGCCAGGCCTGGCAGCCCGGCGTGGCCATGCC | |
| | CAACCTGTACAAGATGCAGAGGATGCTGCTGGAGAAGTGCGACCTGCAGAAC | |
| | TACGGCGACAGCGCCACCCTGCCCAAGGGCATCATGATGAACGTGGCCAAGT | |
| | ACACCCAGCTGTGCCAGTACCTGAACACCCTGACCCTGGCCGTGCCCTACAA | |
| | CATGAGGGTGATCCACTTCGGCGCCGGCAGCGACAAGGGCGTGGCCCCCGG | |
| | CACCGCCGTGCTGAGGCAGTGGCTGCCCACCGGCACCCTGCTGGTGGACAG | |
| | CGACCTGAACGACTTCGTGAGCGACGCCGACAGCACCCTGATCGGCGACTGC | |
| | GCCACCGTGCACACCGCCAACAAGTGGGACCTGATCATCAGCGACATGTACG | |
| | ACCCCAAGACCAAGAACGTGACCAAGGAGAACGACAGCAAGGAGGGCTTCTT | |
| | CACCTACATCTGCGGCTTCATCCAGCAGAAGCTGGCCCTGGGCGGCAGCGTG | |
| | GCCATCAAGATCACCGAGCACAGCTGGAACGCCGACCTGTACAAGCTGATGG | |
| | GCCACTTCGCCTGGTGGACCGCCTTCGTGACCAACGTGAACGCCAGCAGCAG | |
| | CGAGGCCTTCCTGATCGGCTGCAACTACCTGGGCAAGCCCAGGGAGCAGATC | |
| | GACGGCTACGTGATGCACGCCAACTACATCTTCTGGAGGAACACCAACCCCAT | |
| | CCAGCTGAGCAGCTACAGCCTGTTCGACATGAGCAAGTTCCCCCTGAAGCTTG | |
| | ATGA | |
| Pan-CoV | ATGTTCGTGTTCCTGGTGCTGCTGCCCCTGGTGAGCAGCCAGTGCGTGAACC | 111 |
| Vaccine | TGACCACCAGGACCCAGCTGCCCCCCGCCTACACCAACAGCTTCACCAGGGG | |
| # 48 (B5) | CGTGTACTACCCCGACAAGGTGTTCAGGAGCAGCGTGCTGCACAGCACCCAG | |
| | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTATGTA | |
| | CAGCTTCGTGAGCGAGGAGACCGGCACCCTGATCGTGAACAGCGTGCTGCTG | |
| | TTCCTGGCCTTCGTGGTGTTCCTGCTGGTGACCCTGGCCATCCTGACCGCCC | |
| | TGAGGCTGTGCGCCTACTGCTGCAACATCGTGAACGTGAGCCTGGTGAAGCC | |
| | CAGCTTCTACGTGTACAGCAGGGTGAAGAACCTGAACAGCAGCAGGGTGCCC | |
| | GACCTGCTGGTGGGAAGCGGAGCCACGAACTTCTCTCTGTTAAAGCAAGCAG | |
| | GAGATGTTGAAGAAAACCCCGGGCCTATGGCCGACAGCAACGGCACCATCAC | |
| | CGTGGAGGAGCTGAAGAAGCTGCTGGAGCAGTGGAACCTGGTGATCGGCTTC | |
| | CTGTTCCTGACCTGGATCTGCCTGCTGCAGTTCGCCTACGCCAACAGGAACAG | |
| | GTTCCTGTACATCATCAAGCTGATCTTCCTGTGGCTGCTGTGGCCCGTGACCC | |
| | TGGCCTGCTTCGTGCTGGCCGCCGTGTACAGGATCAACTGGATCACCGGCGG | |
| | CATCGCCATCGCCATGGCCTGCCTGGTGGGCCTGATGTGGCTGAGCTACTTCA | |
| | TCGCCAGCTTCAGGCTGTTCGCCAGGACCAGGAGCATGTGGAGCTTCAACCC | |
| | CGAGACCAACATCCTGCTGAACGTGCCCCTGCACGGCACCATCCTGACCAGG | |
| | CCCCTGCTGGAGAGCGAGCTGGTGATCGGCGCCGTGATCCTGAGGGGCCAC | |
| | CTGAGGATCGCCGGCCACCACCTGGGCAGGTGCGACATCAAGGACCTGCCC | |
| | AAGGAGATCACCGTGGCCACCAGCAGGACCCTGAGCTACTACAAGCTGGGCG | |
| | CCAGCCAGAGGGTGGCCGGCGACAGCGGCTTCGCCGCCTACAGCAGGTACA | |
| | GGATCGGCAACTACAAGCTGAACACCGACCACAGCAGCAGCAGCGACAACAT | |
| | CGCCCTGCTGGTGCAGGGAAGCGGAGCCACGAACTTCTCTCTGTTAAAGCAA | |
| | GCAGGAGATGTTGAAGAAAACCCCGGGCCTATGAAGATCATCCTGTTCCTGGC | |
| | CCTGATCACCCTGGCCACCTGCGAGCTGTACCACTACCAGGAGTGCGTGAGG | |
| | GGCACCACCGTGTGATGA | |
| Pan-CoV | ATGTTCGTGTTCCTGGTGCTGCTGCCCCTGGTGAGCAGCCAGTGCGTGAACC | 112 |
| Vaccine | TGACCACCAGGACCCAGCTGCCCCCCGCCTACACCAACAGCTTCACCAGGGG | |
| #49 (B6) | CGTGTACTACCCCGACAAGGTGTTCAGGAGCAGCGTGCTGCACAGCACCCAG | |
| | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCCC | |
| | CACCAAGGTGACCTTCGGCGACGACACCGTGATCGAGGTGCAGGGCTACAAG | |
| | AGCGTGAACATCACCTTCGAGCTGGACGAGAGGATCGACAAGGTGCTGAACG | |
| | AGAAGTGCAGCGCCTACACCGTGGAGCTGGGCACCGAGGTGAACGAGTTCG | |
| | CCTGCGTGGTGGCCGACGCCGTGATCAAGACCCTGCAGCCCGTGAGCGAGC | |
| | TGCTGACCCCCCTGGGCATCGACCTGGACGAGTGGAGCATGGCCACCTACTA | |
| | CCTGTTCGACGAGAGCGGCGAGTTCAAGCTGGCCAGCCACATGTACTGCAGC | |
| | TTCTACCCCCCCGACGAGGACGAGGAGGAGGGCGACTGCGAGGAGGAGGAG | |
| | TTCGAGCCCAGCACCCAGTACGAGTACGGCACCGAGGACGACTACCAGGGCA | |
| | AGCCCCTGGAGTTCGGCGCCACCAGCGCCGCCCTGCAGCCCGAGGAGGAGC | |
| | AGGAGGAGGACTGGCTGGACGACGACAGCCAGCAGACCGTGGGCCAGCAG | |
| | GACGGCAGCGAGGACAACCAGACCACCACCATCCAGACCATCGTGGAGGTGC | |
| | AGCCCCAGCTGGAGATGGAGCTGACCCCCGTGGTGCAGACCATCGAGGTGAA | |
| | CAGCTTCAGCGGCTACCTGAAGCTGACCGACAACGTGTACATCAAGAACGCC | |
| | GACATCGTGGAGGAGGCCAAGAAGGTGAAGCCCACCGTGGTGGTGAACGCC | |
| | GCCAACGTGTACCTGAAGCACGGCGGCGGCGTGGCCGGCGCCCTGAACAAG | |
| | GCCACCAACAACGCCATGCAGGTGGAGAGCGACGACTACATCGCCACCAACG | |
| | GCCCCCTGAAGGTGGGCGGCAGCTGCGTGCTGAGCGGCCACAACCTGGCCA | |
| | AGCACTGCCTGCACGTGGTGGGCCCCAACGTGAACAAGGGCGAGGACATCC | |
| | AGCTGCTGAAGAGCGCCTACGAGAACTTCAACCAGCACGAGGTGCTGCTGGC | |
| | CCCCCTGCTGAGCGCCGGCATCTTCGGCGCCGACCCCATCCACAGCCTGAG | |
| | GGTGTGCGTGGACACCGTGAGGACCAACGTGTACCTGGCCGTGTTCGACAAG | |
| | AACCTGTACGACAAGCTGGTGAGCAGCTTCCTGGAGATGAAGAGCGAGAAGC | |
| | AGGTGGAGCAGAAGATCGCCGAGATCCCCAAGGAGGAGGTGAAGCCCTTCAT | |
| | CACCGAGAGCAAGCCCAGCGTGGAGCAGAGGAAGCAGGACGACAAGAAGAT | |
| | CAAGGCCTGCGTGGAGGAGGTGACCACCACCCTGGAGGAGACCAAGTTCCT | |
| | GACCGAGAACCTGCTGCTGTACATCGACATCAACGGCAACCTGCACCCCGAC | |
| | AGCGCCACCCTGGTGAGCGACATCGACATCACCTTCCTGAAGAAGGACGCCC | |
| | CCTACATCGTGGGCGACGTGGTGCAGGAGGGCGTGCTGACCGCCGTGGTGA | |
| | TCCCCACCAAGAAGGCCGGCGGCACCACCGAGATGCTGGCCAAGGCCCTGA | |
| | GGAAGGTGCCCACCGACAACTACATCACCACCTACCCCGGCCAGGGCCTGAA | |
| | CGGCTACACCGTGGAGGAGGCCAAGACCGTGCTGAAGAAGTGCAAGAGCGC | |
| | CTTCTACATCCTGCCCAGCATCATCAGCAACGAGAAGCAGGAGATCCTGGGCA | |
| | CCGTGAGCTGGAACCTGAGGGAGATGCTGGCCCACGCCGAGGAGACCAGGA | |
| | AGCTGATGCCCGTGTGCGTGGAGACCAAGGCCATCGTGAGCACCATCCAGAG | |
| | GAAGTACAAGGGCATCAAGATCCAGGAGGGCGTGGTGGACTACGGCGCCAGG | |
| | TTCTACTTCTACACCAGCAAGACCACCGTGGCCAGCCTGATCAACACCCTGAA | |
| | CGACCTGAACGAGACCCTGGTGACCATGCCCCTGGGCTACGTGACCCACGGC | |
| | CTGAACCTGGAGGAGGCCGCCAGGTACATGAGGAGCCTGAAGGTGCCCGCC | |
| | ACCGTGAGCGTGAGCAGCCCCGACGCCGTGACCGCCTACAACGGCTACCTGA | |
| | CCAGCAGCAGCAAGACCCCCGAGGAGCACTTCATCGAGACCATCAGCCTGGC | |
| | CGGCAGCTACAAGGACTGGAGCTACAGCGGCCAGAGCACCCAGCTGGGCAT | |
| | CGAGTTCCTGAAGAGGGGCGACAAGAGCGTGTACTACACCAGCAACCCCACC | |
| | ACCTTCCACCTGGACGGCGAGGTGATCACCTTCGACAACCTGAAGACCCTGC | |
| | TGAGCCTGAGGGAGGTGAGGACCATCAAGGTGTTCACCACCGTGGACAACAT | |
| | CAACCTGCACGGAAGCGGAGCCACGAACTTCTCTCTGTTAAAGCAAGCAGGA | |
| | GATGTTGAAGAAAACCCCGGGCCTAGCGGCTTCAGGAAGATGGCCTTCCCCA | |
| | GCGGCAAGGTGGAGGGCTGCATGGTGCAGGTGACCTGCGGCACCACCACCC | |
| | TGAACGGCCTGTGGCTGGACGACGTGGTGTACTGCCCCAGGCACGTGATCTG | |
| | CACCAGCGAGGACATGCTGAACCCCAACTACGAGGACCTGCTGATCAGGAAG | |
| | AGCAACCACAACTTCCTGGTGCAGGCCGGCAACGTGCAGCTGAGGGTGATCG | |
| | GCCACAGCATGCAGAACTGCGTGCTGAAGCTGAAGGTGGACACCGCCAACCC | |
| | CAAGACCCCCAAGTACAAGTTCGTGAGGATCCAGCCCGGCCAGACCTTCAGC | |
| | GTGCTGGCCTGCTACAACGGCAGCCCCAGCGGCGTGTACCAGTGCGCCATGA | |
| | GGCCCAACTTCACCATCAAGGGCAGCTTCCTGAACGGCAGCTGCGGCAGCGT | |
| | GGGCTTCAACATCGACTACGACTGCGTGAGCTTCTGCTACATGCACCACATGG | |
| | AGCTGCCCACCGGCGTGCACGCCGGCACCGACCTGGAGGGCAACTTCTACG | |
| | GCCCCTTCGTGGACAGGCAGACCGCCCAGGCCGCCGGCACCGACACCACCA | |
| | TCACCGTGAACGTGCTGGCCTGGCTGTACGCCGCCGTGATCAACGGCGACAG | |
| | GTGGTTCCTGAACAGGTTCACCACCACCCTGAACGACTTCAACCTGGTGGCC | |
| | ATGAAGTACAACTACGAGCCCCTGACCCAGGACCACGTGGACATCCTGGGCC | |
| | CCCTGAGCGCCCAGACCGGCATCGCCGTGCTGGACATGTGCGCCAGCCTGA | |
| | AGGAGCTGCTGCAGAACGGCATGAACGGCAGGACCATCCTGGGCAGCGCCC | |
| | TGCTGGAGGACGAGTTCACCCCCTTCGACGTGGTGAGGCAGTGCAGCGGCG | |
| | TGACCTTCCAGGGAAGCGGAGCCACGAACTTCTCTCTGTTAAAGCAAGCAGG | |
| | AGATGTTGAAGAAAACCCCGGGCCTAGCGCCGACGCCCAGAGCTTCCTGAAC | |
| | AGGGTGTGCGGCGTGAGCGCCGCCAGGCTGACCCCCTGCGGCACCGGCAC | |
| | CAGCACCGACGTGGTGTACAGGGCCTTCGACATCTACAACGACAAGGTGGCC | |
| | GGCTTCGCCAAGTTCCTGAAGACCAACTGCTGCAGGTTCCAGGAGAAGGACG | |
| | AGGACGACAACCTGATCGACAGCTACTTCGTGGTGAAGAGGCACACCTTCAG | |
| | CAACTACCAGCACGAGGAGACCATCTACAACCTGCTGAAGGACTGCCCCGCC | |
| | GTGGCCAAGCACGACTTCTTCAAGTTCAGGATCGACGGCGACATGGTGCCCC | |
| | ACATCAGCAGGCAGAGGCTGACCAAGTACACCATGGCCGACCTGGTGTACGC | |
| | CCTGAGGCACTTCGACGAGGGCAACTGCGACACCCTGAAGGAGATCCTGGTG | |
| | ACCTACAACTGCTGCGACGACGACTACTTCAACAAGAAGGACTGGTACGACTT | |
| | CGTGGAGAACCCCGACATCCTGAGGGTGTACGCCAACCTGGGCGAGAGGGT | |
| | GAGGCAGGCCCTGCTGAAGACCGTGCAGTTCTGCGACGCCATGAGGAACGC | |
| | CGGCATCGTGGGCGTGCTGACCCTGGACAACCAGGACCTGAACGGCAACTG | |
| | GTACGACTTCGGCGACTTCATCCAGACCACCCCCGGCAGCGGCGTGCCCGTG | |
| | GTGGACAGCTACTACAGCCTGCTGATGCCCATCCTGACCCTGACCAGGGCCC | |
| | TGACCGCCGAGAGCCACGTGGACACCGACCTGACCAAGCCCTACATCAAGTG | |
| | GGACCTGCTGAAGTACGACTTCACCGAGGAGAGGCTGAAGCTGTTCGACAGG | |
| | TACTTCAAGTACTGGGACCAGACCTACCACCCCAACTGCGTGAACTGCCTGGA | |
| | CGACAGGTGCATCCTGCACTGCGCCAACTTCAACGTGCTGTTCAGCACCGTG | |
| | TTCCCCCCCACCAGCTTCGGCCCCCTGGTGAGGAAGATCTTCGTGGACGGCG | |
| | TGCCCTTCGTGGTGAGCACCGGCTACCACTTCAGGGAGCTGGGCGTGGTGCA | |
| | CAACCAGGACGTGAACCTGCACAGCAGCAGGCTGAGCTTCAAGGAGCTGCTG | |
| | GTGTACGCCGCCGACCCCGCCATGCACGCCGCCAGCGGCAACCTGCTGCTG | |
| | GACAAGAGGACCACCTGCTTCAGCGTGGCCGCCCTGACCAACAACGTGGCCT | |
| | TCCAGACCGTGAAGCCCGGCAACTTCAACAAGGACTTCTACGACTTCGCCGT | |
| | GAGCAAGGGCTTCTTCAAGGAGGGCAGCAGCGTGGAGCTGAAGCACTTCTTC | |
| | TTCGCCCAGGACGGCAACGCCGCCATCAGCGACTACGACTACTACAGGTACAA | |
| | CCTGCCCACCATGTGCGACATCAGGCAGCTGCTGTTCGTGGTGGAGGTGGTG | |
| | GACAAGTACTTCGACTGCTACGACGGCGGCTGCATCAACGCCAACCAGGTGAT | |
| | CGTGAACAACCTGGACAAGAGCGCCGGCTTCCCCTTCAACAAGTGGGGCAAG | |
| | GCCAGGCTGTACTACGACAGCATGAGCTACGAGGACCAGGACGCCCTGTTCG | |
| | CCTACACCAAGAGGAACGTGATCCCCACCATCACCCAGATGAACCTGAAGTAC | |
| | GCCATCAGCGCCAAGAACAGGGCCAGGACCGTGGCCGGCGTGAGCATCTGC | |
| | AGCACCATGACCAACAGGCAGTTCCACCAGAAGCTGCTGAAGAGCATCGCCG | |
| | CCACCAGGGGCGCCACCGTGGTGATCGGCACCAGCAAGTTCTACGGCGGCT | |
| | GGCACAACATGCTGAAGACCGTGTACAGCGACGTGGAGAACCCCCACCTGAT | |
| | GGGCTGGGACTACCCCAAGTGCGACAGGGCCATGCCCAACATGCTGAGGATC | |
| | ATGGCCAGCCTGGTGCTGGCCAGGAAGCACACCACCTGCTGCAGCCTGAGC | |
| | CACAGGTTCTACAGGCTGGCCAACGAGTGCGCCCAGGTGCTGAGCGAGATGG | |
| | TGATGTGCGGCGGCAGCCTGTACGTGAAGCCCGGCGGCACCAGCAGCGGCG | |
| | ACGCCACCACCGCCTACGCCAACAGCGTGTTCAACATCTGCCAGGCCGTGAC | |
| | CGCCAACGTGAACGCCCTGCTGAGCACCGACGGCAACAAGATCGCCGACAAG | |
| | TACGTGAGGAACCTGCAGCACAGGCTGTACGAGTGCCTGTACAGGAACAGGG | |
| | ACGTGGACACCGACTTCGTGAACGAGTTCTACGCCTACCTGAGGAAGCACTTC | |
| | AGCATGATGATCCTGAGCGACGACGCCGTGGTGTGCTTCAACAGCACCTACG | |
| | CCAGCCAGGGCCTGGTGGCCAGCATCAAGAACTTCAAGAGCGTGCTGTACTA | |
| | CCAGAACAACGTGTTCATGAGCGAGGCCAAGTGCTGGACCGAGACCGACCTG | |
| | ACCAAGGGCCCCCACGAGTTCTGCAGCCAGCACACCATGCTGGTGAAGCAGG | |
| | GCGACGACTACGTGTACCTGCCCTACCCCGACCCCAGCAGGATCCTGGGCGC | |
| | CGGCTGCTTCGTGGACGACATCGTGAAGACCGACGGCACCCTGATGATCGAG | |
| | AGGTTCGTGAGCCTGGCCATCGACGCCTACCCCCTGACCAAGCACCCCAACC | |
| | AGGAGTACGCCGACGTGTTCCACCTGTACCTGCAGTACATCAGGAAGCTGCAC | |
| | GACGAGCTGACCGGCCACATGCTGGACATGTACAGCGTGATGCTGACCAACG | |
| | ACAACACCAGCAGGTACTGGGAGCCCGAGTTCTACGAGGCCATGTACACCCC | |
| | CCACACCGTGCTGCAGTGATGA | |
| Pan-CoV | ATGTTCGTGTTCCTGGTGCTGCTGCCCCTGGTGAGCAGCCAGTGCGTGAACC | 113 |
| Vaccine | TGACCACCAGGACCCAGCTGCCCCCCGCCTACACCAACAGCTTCACCAGGGG | |
| #50 | CGTGTACTACCCCGACAAGGTGTTCAGGAGCAGCGTGCTGCACAGCACCCAG | |
| (B6.1) | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTGCCCC | |
| | CACCAAGGTGACCTTCGGCGACGACACCGTGATCGAGGTGCAGGGCTACAAG | |
| | AGCGTGAACATCACCTTCGAGCTGGACGAGAGGATCGACAAGGTGCTGAACG | |
| | AGAAGTGCAGCGCCTACACCGTGGAGCTGGGCACCGAGGTGAACGAGTTCG | |
| | CCTGCGTGGTGGCCGACGCCGTGATCAAGACCCTGCAGCCCGTGAGCGAGC | |
| | TGCTGACCCCCCTGGGCATCGACCTGGACGAGTGGAGCATGGCCACCTACTA | |
| | CCTGTTCGACGAGAGCGGCGAGTTCAAGCTGGCCAGCCACATGTACTGCAGC | |
| | TTCTACCCCCCCGACGAGGACGAGGAGGAGGGCGACTGCGAGGAGGAGGAG | |
| | TTCGAGCCCAGCACCCAGTACGAGTACGGCACCGAGGACGACTACCAGGGCA | |
| | AGCCCCTGGAGTTCGGCGCCACCAGCGCCGCCCTGCAGCCCGAGGAGGAGC | |
| | AGGAGGAGGACTGGCTGGACGACGACAGCCAGCAGACCGTGGGCCAGCAG | |
| | GACGGCAGCGAGGACAACCAGACCACCACCATCCAGACCATCGTGGAGGTGC | |
| | AGCCCCAGCTGGAGATGGAGCTGACCCCCGTGGTGCAGACCATCGAGGTGAA | |
| | CAGCTTCAGCGGCTACCTGAAGCTGACCGACAACGTGTACATCAAGAACGCC | |
| | GACATCGTGGAGGAGGCCAAGAAGGTGAAGCCCACCGTGGTGGTGAACGCC | |
| | GCCAACGTGTACCTGAAGCACGGCGGCGGCGTGGCCGGCGCCCTGAACAAG | |
| | GCCACCAACAACGCCATGCAGGTGGAGAGCGACGACTACATCGCCACCAACG | |
| | GCCCCCTGAAGGTGGGCGGCAGCTGCGTGCTGAGCGGCCACAACCTGGCCA | |
| | AGCACTGCCTGCACGTGGTGGGCCCCAACGTGAACAAGGGCGAGGACATCC | |
| | AGCTGCTGAAGAGCGCCTACGAGAACTTCAACCAGCACGAGGTGCTGCTGGC | |
| | CCCCCTGCTGAGCGCCGGCATCTTCGGCGCCGACCCCATCCACAGCCTGAG | |
| | GGTGTGCGTGGACACCGTGAGGACCAACGTGTACCTGGCCGTGTTCGACAAG | |
| | AACCTGTACGACAAGCTGGTGAGCAGCTTCCTGGAGATGAAGAGCGAGAAGC | |
| | AGGTGGAGCAGAAGATCGCCGAGATCCCCAAGGAGGAGGTGAAGCCCTTCAT | |
| | CACCGAGAGCAAGCCCAGCGTGGAGCAGAGGAAGCAGGACGACAAGAAGAT | |
| | CAAGGCCTGCGTGGAGGAGGTGACCACCACCCTGGAGGAGACCAAGTTCCT | |
| | GACCGAGAACCTGCTGCTGTACATCGACATCAACGGCAACCTGCACCCCGAC | |
| | AGCGCCACCCTGGTGAGCGACATCGACATCACCTTCCTGAAGAAGGACGCCC | |
| | CCTACATCGTGGGCGACGTGGTGCAGGAGGGCGTGCTGACCGCCGTGGTGA | |
| | TCCCCACCAAGAAGGCCGGCGGCACCACCGAGATGCTGGCCAAGGCCCTGA | |
| | GGAAGGTGCCCACCGACAACTACATCACCACCTACCCCGGCCAGGGCCTGAA | |
| | CGGCTACACCGTGGAGGAGGCCAAGACCGTGCTGAAGAAGTGCAAGAGCGC | |
| | CTTCTACATCCTGCCCAGCATCATCAGCAACGAGAAGCAGGAGATCCTGGGCA | |
| | CCGTGAGCTGGAACCTGAGGGAGATGCTGGCCCACGCCGAGGAGACCAGGA | |
| | AGCTGATGCCCGTGTGCGTGGAGACCAAGGCCATCGTGAGCACCATCCAGAG | |
| | GAAGTACAAGGGCATCAAGATCCAGGAGGGCGTGGTGGACTACGGCGCCAGG | |
| | TTCTACTTCTACACCAGCAAGACCACCGTGGCCAGCCTGATCAACACCCTGAA | |
| | CGACCTGAACGAGACCCTGGTGACCATGCCCCTGGGCTACGTGACCCACGGC | |
| | CTGAACCTGGAGGAGGCCGCCAGGTACATGAGGAGCCTGAAGGTGCCCGCC | |
| | ACCGTGAGCGTGAGCAGCCCCGACGCCGTGACCGCCTACAACGGCTACCTGA | |
| | CCAGCAGCAGCAAGACCCCCGAGGAGCACTTCATCGAGACCATCAGCCTGGC | |
| | CGGCAGCTACAAGGACTGGAGCTACAGCGGCCAGAGCACCCAGCTGGGCAT | |
| | CGAGTTCCTGAAGAGGGGCGACAAGAGCGTGTACTACACCAGCAACCCCACC | |
| | ACCTTCCACCTGGACGGCGAGGTGATCACCTTCGACAACCTGAAGACCCTGC | |
| | TGAGCCTGAGGGAGGTGAGGACCATCAAGGTGTTCACCACCGTGGACAACAT | |
| | CAACCTGCACTGATGA | |
| Pan-CoV | ATGTTCGTGTTCCTGGTGCTGCTGCCCCTGGTGAGCAGCCAGTGCGTGAACC | 114 |
| Vaccine | TGACCACCAGGACCCAGCTGCCCCCCGCCTACACCAACAGCTTCACCAGGGG | |
| #51 | CGTGTACTACCCCGACAAGGTGTTCAGGAGCAGCGTGCTGCACAGCACCCAG | |
| (B6.2) | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGG | |
| | CTTCAGGAAGATGGCCTTCCCCAGCGGCAAGGTGGAGGGCTGCATGGTGCAG | |
| | GTGACCTGCGGCACCACCACCCTGAACGGCCTGTGGCTGGACGACGTGGTG | |
| | TACTGCCCCAGGCACGTGATCTGCACCAGCGAGGACATGCTGAACCCCAACTA | |
| | CGAGGACCTGCTGATCAGGAAGAGCAACCACAACTTCCTGGTGCAGGCCGGC | |
| | AACGTGCAGCTGAGGGTGATCGGCCACAGCATGCAGAACTGCGTGCTGAAGC | |
| | TGAAGGTGGACACCGCCAACCCCAAGACCCCCAAGTACAAGTTCGTGAGGAT | |
| | CCAGCCCGGCCAGACCTTCAGCGTGCTGGCCTGCTACAACGGCAGCCCCAG | |
| | CGGCGTGTACCAGTGCGCCATGAGGCCCAACTTCACCATCAAGGGCAGCTTC | |
| | CTGAACGGCAGCTGCGGCAGCGTGGGCTTCAACATCGACTACGACTGCGTGA | |
| | GCTTCTGCTACATGCACCACATGGAGCTGCCCACCGGCGTGCACGCCGGCAC | |
| | CGACCTGGAGGGCAACTTCTACGGCCCCTTCGTGGACAGGCAGACCGCCCA | |
| | GGCCGCCGGCACCGACACCACCATCACCGTGAACGTGCTGGCCTGGCTGTAC | |
| | GCCGCCGTGATCAACGGCGACAGGTGGTTCCTGAACAGGTTCACCACCACCC | |
| | TGAACGACTTCAACCTGGTGGCCATGAAGTACAACTACGAGCCCCTGACCCAG | |
| | GACCACGTGGACATCCTGGGCCCCCTGAGCGCCCAGACCGGCATCGCCGTG | |
| | CTGGACATGTGCGCCAGCCTGAAGGAGCTGCTGCAGAACGGCATGAACGGCA | |
| | GGACCATCCTGGGCAGCGCCCTGCTGGAGGACGAGTTCACCCCCTTCGACGT | |
| | GGTGAGGCAGTGCAGCGGCGTGACCTTCCAGTGATGA | |
| Pan-CoV | ATGTTCGTGTTCCTGGTGCTGCTGCCCCTGGTGAGCAGCCAGTGCGTGAACC | 115 |
| Vaccine | TGACCACCAGGACCCAGCTGCCCCCCGCCTACACCAACAGCTTCACCAGGGG | |
| #52 | CGTGTACTACCCCGACAAGGTGTTCAGGAGCAGCGTGCTGCACAGCACCCAG | |
| (B6.3) | GACCTGTTCCTGCCCTTCTTCAGCAACGTGACCTGGTTCCACGCCATCCACGT | |
| | GAGCGGCACCAACGGCACCAAGAGGTTCGACAACCCCGTGCTGCCCTTCAAC | |
| | GACGGCGTGTACTTCGCCAGCACCGAGAAGAGCAACATCATCAGGGGCTGGA | |
| | TCTTCGGCACCACCCTGGACAGCAAGACCCAGAGCCTGCTGATCGTGAACAA | |
| | CGCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAGTT | |
| | CAGGGTGTACAGCAGCGCCAACAACTGCACCTTCGAGTACGTGAGCCAGCCC | |
| | TTCCTGATGGACCTGGAGGGCAAGCAGGGCAACTTCAAGAACCTGAGGGAGT | |
| | TCGTGTTCAAGAACATCGACGGCTACTTCAAGATCTACAGCAAGCACACCCCC | |
| | ATCAACCTGGTGAGGGACCTGCCCCAGGGCTTCAGCGCCCTGGAGCCCCTG | |
| | GTGGACCTGCCCATCGGCATCAACATCACCAGGTTCCAGACCCTGCTGGCCC | |
| | TGCACAGGAGCTACCTGACCCCCGGCGACAGCAGCAGCGGCTGGACCGCCG | |
| | GCGCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACCCC | |
| | CTGAGCGAGACCAAGTGCACCCTGAAGAGCTTCACCGTGGAGAAGGGCATCT | |
| | ACCAGACCAGCAACTTCAGGGTGCAGCCCACCGAGAGCATCGTGAGGTTCCC | |
| | CAACATCACCAACCTGTGCCCCTTCGGCGAGGTGTTCAACGCCACCAGGTTC | |
| | GCCAGCGTGTACGCCTGGAACAGGAAGAGGATCAGCAACTGCGTGGCCGACT | |
| | ACAGCGTGCTGTACAACAGCGCCAGCTTCAGCACCTTCAAGTGCTACGGCGT | |
| | GAGCCCCACCAAGCTGAACGACCTGTGCTTCACCAACGTGTACGCCGACAGC | |
| | TTCGTGATCAGGGGCGACGAGGTGAGGCAGATCGCCCCCGGCCAGACCGGC | |
| | AAGATCGCCGACTACAACTACAAGCTGCCCGACGACTTCACCGGCTGCGTGAT | |
| | CGCCTGGAACAGCAACAACCTGGACAGCAAGGTGGGCGGCAACTACAACTAC | |
| | CTGTACAGGCTGTTCAGGAAGAGCAACCTGAAGCCCTTCGAGAGGGACATCA | |
| | GCACCGAGATCTACCAGGCCGGCAGCACCCCCTGCAACGGCGTGGAGGGCT | |
| | TCAACTGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAAGT | |
| | GCGTGAACTTCAACTTCAACGGCCTGACCGGCACCGGCGTGCTGACCGAGAG | |
| | CAACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCCCCT | |
| | GCAGCTTCGGCGGCGTGAGCGTGATCACCCCCGGCACCAACACCAGCAACC | |
| | AGGTGGCCGTGCTGTACCAGGACGTGAACTGCACCGAGGTGCCCGTGGCCAT | |
| | CCACGCCGACCAGCTGACCCCCACCTGGAGGGTGTACAGCACCGGCAGCAA | |
| | CGTGTTCCAGACCAGGGCCGGCTGCCTGATCGGCGCCGAGCACGTGAACAA | |
| | CAGCTACGAGTGCGACATCCCCATCGGCGCCGGCATCTGCGCCAGCTACCAG | |
| | ACCCAGACCAACAGCCCCGGCAGCGCCAGCAGCGTGGCCAGCCAGAGCATC | |
| | ATCGCCTACACCATGAGCCTGGGCGCCGAGAACAGCGTGGCCTACAGCAACA | |
| | ACAGCATCGCCATCCCCACCAACTTCACCATCAGCGTGACCACCGAGATCCTG | |
| | CCCGTGAGCATGACCAAGACCAGCGTGGACTGCACCATGTACATCTGCGGCG | |
| | ACAGCACCGAGTGCAGCAACCTGCTGCTGCAGTACGGCAGCTTCTGCACCCA | |
| | GCTGAACAGGGCCCTGACCGGCATCGCCGTGGAGCAGGACAAGAACACCCA | |
| | GGAGGTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCCAGCA | |
| | AGAGGAGCCCCATCGAGGACCTGCTGTTCAACAAGGTGACCCTGGCCGACGC | |
| | CGGCTTCATCAAGCAGTACGGCGACTGCCTGGGCGACATCGCCGCCAGGGAC | |
| | CTGATCTGCGCCCAGAAGTTCAACGGCCTGACCGTGCTGCCCCCCCTGCTGA | |
| | CCGACGAGATGATCGCCCAGTACACCAGCGCCCTGCTGGCCGGCACCATCAC | |
| | CAGCGGCTGGACCTTCGGCGCCGGCCCCGCCCTGCAGATCCCCTTCCCCAT | |
| | GCAGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATCC | |
| | AGGACAGCCTGAGCAGCACCCCCAGCGCCCTGGGCAAGCTGCAGGACGTGG | |
| | TGAACCAGAACGCCCAGGCCCTGAACACCCTGGTGAAGCAGCTGAGCAGCAA | |
| | CTTCGGCGCCATCAGCAGCGTGCTGAACGACATCCTGAGCAGGCTGGACCCC | |
| | CCCGAGGCCGAGGTGCAGATCGACAGGCTGATCACCGGCAGGCTGCAGAGC | |
| | CTGCAGACCTACGTGACCCAGCAGCTGATCAGGGCCGCCGAGATCAGGGCCA | |
| | GCGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAGAG | |
| | CGCCCCCCACGGCGTGGTGTTCCTGCACGTGACCTACGTGCCCGCCCAGGA | |
| | GAAGAACTTCACCACCGCCCCCGCCATCTGCCACGACGGCAAGGCCCACTTC | |
| | CCCAGGGAGGGCGTGTTCGTGAGCAACGGCACCCACTGGTTCGTGACCCAG | |
| | AGGAACTTCTACGAGCCCCAGATCATCACCACCGACAACACCTTCGTGAGCGG | |
| | CAACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAACC | |
| | ACACCAGCCCCGACGTGGACCTGGGCGACATCAGCGGCATCAACGCCAGCG | |
| | TGGTGAACATCCAGAAGGAGATCGACAGGCTGAACGAGGTGGCCAAGAACCT | |
| | GAACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATCGT | |
| | GATGGTGACCATCATGCTGTGCTGCATGACCAGCTGCTGCAGCTGCCTGAAG | |
| | GGCTGCTGCAGCTGCGGCAGCTGCTGCAAGTTCGACGAGGACGACAGCGAG | |
| | CCCGTGCTGAAGGGCGTGAAGCTGCACTACACCGGAAGCGGAGCCACGAAC | |
| | TTCTCTCTGTTAAAGCAAGCAGGAGATGTTGAAGAAAACCCCGGGCCTAGCGC | |
| | CGACGCCCAGAGCTTCCTGAACAGGGTGTGCGGCGTGAGCGCCGCCAGGCT | |
| | GACCCCCTGCGGCACCGGCACCAGCACCGACGTGGTGTACAGGGCCTTCGA | |
| | CATCTACAACGACAAGGTGGCCGGCTTCGCCAAGTTCCTGAAGACCAACTGCT | |
| | GCAGGTTCCAGGAGAAGGACGAGGACGACAACCTGATCGACAGCTACTTCGT | |
| | GGTGAAGAGGCACACCTTCAGCAACTACCAGCACGAGGAGACCATCTACAAC | |
| | CTGCTGAAGGACTGCCCCGCCGTGGCCAAGCACGACTTCTTCAAGTTCAGGA | |
| | TCGACGGCGACATGGTGCCCCACATCAGCAGGCAGAGGCTGACCAAGTACAC | |
| | CATGGCCGACCTGGTGTACGCCCTGAGGCACTTCGACGAGGGCAACTGCGAC | |
| | ACCCTGAAGGAGATCCTGGTGACCTACAACTGCTGCGACGACGACTACTTCAA | |
| | CAAGAAGGACTGGTACGACTTCGTGGAGAACCCCGACATCCTGAGGGTGTAC | |
| | GCCAACCTGGGCGAGAGGGTGAGGCAGGCCCTGCTGAAGACCGTGCAGTTC | |
| | TGCGACGCCATGAGGAACGCCGGCATCGTGGGCGTGCTGACCCTGGACAAC | |
| | CAGGACCTGAACGGCAACTGGTACGACTTCGGCGACTTCATCCAGACCACCC | |
| | CCGGCAGCGGCGTGCCCGTGGTGGACAGCTACTACAGCCTGCTGATGCCCAT | |
| | CCTGACCCTGACCAGGGCCCTGACCGCCGAGAGCCACGTGGACACCGACCT | |
| | GACCAAGCCCTACATCAAGTGGGACCTGCTGAAGTACGACTTCACCGAGGAG | |
| | AGGCTGAAGCTGTTCGACAGGTACTTCAAGTACTGGGACCAGACCTACCACCC | |
| | CAACTGCGTGAACTGCCTGGACGACAGGTGCATCCTGCACTGCGCCAACTTC | |
| | AACGTGCTGTTCAGCACCGTGTTCCCCCCCACCAGCTTCGGCCCCCTGGTGA | |
| | GGAAGATCTTCGTGGACGGCGTGCCCTTCGTGGTGAGCACCGGCTACCACTT | |
| | CAGGGAGCTGGGCGTGGTGCACAACCAGGACGTGAACCTGCACAGCAGCAG | |
| | GCTGAGCTTCAAGGAGCTGCTGGTGTACGCCGCCGACCCCGCCATGCACGCC | |
| | GCCAGCGGCAACCTGCTGCTGGACAAGAGGACCACCTGCTTCAGCGTGGCC | |
| | GCCCTGACCAACAACGTGGCCTTCCAGACCGTGAAGCCCGGCAACTTCAACA | |
| | AGGACTTCTACGACTTCGCCGTGAGCAAGGGCTTCTTCAAGGAGGGCAGCAG | |
| | CGTGGAGCTGAAGCACTTCTTCTTCGCCCAGGACGGCAACGCCGCCATCAGC | |
| | GACTACGACTACTACAGGTACAACCTGCCCACCATGTGCGACATCAGGCAGCT | |
| | GCTGTTCGTGGTGGAGGTGGTGGACAAGTACTTCGACTGCTACGACGGCGGC | |
| | TGCATCAACGCCAACCAGGTGATCGTGAACAACCTGGACAAGAGCGCCGGCT | |
| | TCCCCTTCAACAAGTGGGGCAAGGCCAGGCTGTACTACGACAGCATGAGCTAC | |
| | GAGGACCAGGACGCCCTGTTCGCCTACACCAAGAGGAACGTGATCCCCACCA | |
| | TCACCCAGATGAACCTGAAGTACGCCATCAGCGCCAAGAACAGGGCCAGGAC | |
| | CGTGGCCGGCGTGAGCATCTGCAGCACCATGACCAACAGGCAGTTCCACCAG | |
| | AAGCTGCTGAAGAGCATCGCCGCCACCAGGGGCGCCACCGTGGTGATCGGC | |
| | ACCAGCAAGTTCTACGGCGGCTGGCACAACATGCTGAAGACCGTGTACAGCG | |
| | ACGTGGAGAACCCCCACCTGATGGGCTGGGACTACCCCAAGTGCGACAGGG | |
| | CCATGCCCAACATGCTGAGGATCATGGCCAGCCTGGTGCTGGCCAGGAAGCA | |
| | CACCACCTGCTGCAGCCTGAGCCACAGGTTCTACAGGCTGGCCAACGAGTGC | |
| | GCCCAGGTGCTGAGCGAGATGGTGATGTGCGGCGGCAGCCTGTACGTGAAG | |
| | CCCGGCGGCACCAGCAGCGGCGACGCCACCACCGCCTACGCCAACAGCGTG | |
| | TTCAACATCTGCCAGGCCGTGACCGCCAACGTGAACGCCCTGCTGAGCACCG | |
| | ACGGCAACAAGATCGCCGACAAGTACGTGAGGAACCTGCAGCACAGGCTGTA | |
| | CGAGTGCCTGTACAGGAACAGGGACGTGGACACCGACTTCGTGAACGAGTTC | |
| | TACGCCTACCTGAGGAAGCACTTCAGCATGATGATCCTGAGCGACGACGCCGT | |
| | GGTGTGCTTCAACAGCACCTACGCCAGCCAGGGCCTGGTGGCCAGCATCAAG | |
| | AACTTCAAGAGCGTGCTGTACTACCAGAACAACGTGTTCATGAGCGAGGCCAA | |
| | GTGCTGGACCGAGACCGACCTGACCAAGGGCCCCCACGAGTTCTGCAGCCA | |
| | GCACACCATGCTGGTGAAGCAGGGCGACGACTACGTGTACCTGCCCTACCCC | |
| | GACCCCAGCAGGATCCTGGGCGCCGGCTGCTTCGTGGACGACATCGTGAAGA | |
| | CCGACGGCACCCTGATGATCGAGAGGTTCGTGAGCCTGGCCATCGACGCCTA | |
| | CCCCCTGACCAAGCACCCCAACCAGGAGTACGCCGACGTGTTCCACCTGTAC | |
| | CTGCAGTACATCAGGAAGCTGCACGACGAGCTGACCGGCCACATGCTGGACA | |
| | TGTACAGCGTGATGCTGACCAACGACAACACCAGCAGGTACTGGGAGCCCGA | |
| | GTTCTACGAGGCCATGTACACCCCCCACACCGTGCTGCAGTGATGA | |
| Pan-CoV | ATGAGCGACAACGGCCCCCAGAACCAGAGGAACGCCCCCAGGATCACCTTCG | 116 |
| Vaccine | GCGGCCCCAGCGACAGCACCGGCAGCAACCAGAACGGCGAGAGGAGCGGC | |
| #53 (B3) | GCCAGGAGCAAGCAGAGGAGGCCCCAGGGCCTGCCCAACAACACCGCCAGC | |
| | TGGTTCACCGCCCTGACCCAGCACGGCAAGGAGGACCTGAAGTTCCCCAGG | |
| | GGCCAGGGCGTGCCCATCAACACCAACAGCAGCCCCGACGACCAGATCGGC | |
| | TACTACAGGAGGGCCACCAGGAGGATCAGGGGCGGCGACGGCAAGATGAAG | |
| | GACCTGAGCCCCAGGTGGTACTTCTACTACCTGGGCACCGGCCCCGAGGCCG | |
| | GCCTGCCCTACGGCGCCAACAAGGACGGCATCATCTGGGTGGCCACCGAGG | |
| | GCGCCCTGAACACCCCCAAGGACCACATCGGCACCAGGAACCCCGCCAACAA | |
| | CGCCGCCATCGTGCTGCAGCTGCCCCAGGGCACCACCCTGCCCAAGGGCTT | |
| | CTACGCCGAGGGCAGCAGGGGCGGCAGCCAGGCCAGCAGCAGGAGCAGCA | |
| | GCAGGAGCAGGAACAGCAGCAGGAACAGCACCCCCGGCAGCAGCAGGGGC | |
| | ACCAGCCCCGCCAGGATGGCCGGCAACGGCGGCGACGCCGCCCTGGCCCT | |
| | GCTGCTGCTGGACAGGCTGAACCAGCTGGAGAGCAAGATGAGCGGCAAGGG | |
| | CCAGCAGCAGCAGGGCCAGACCGTGACCAAGAAGAGCGCCGCCGAGGCCAG | |
| | CAAGAAGCCCAGGCAGAAGAGGACCGCCACCAAGGCCTACAACGTGACCCA | |
| | GGCCTTCGGCAGGAGGGGCCCCGAGCAGACCCAGGGCAACTTCGGCGACCA | |
| | GGAGCTGATCAGGCAGGGCACCGACTACAAGCACTGGCCCCAGATCGCCCAG | |
| | TTCGCCCCCAGCGCCAGCGCCTTCTTCGGCATGAGCAGGATCGGCATGGAGG | |
| | TGACCCCCAGCGGCACCTGGCTGACCTACACCGGCGCCATCAAGCTGGACGA | |
| | CAAGGACCCCAACTTCAAGGACCAGGTGATCCTGCTGAACAAGCACATCGAC | |
| | GCCTACAAGACCTTCCCCCCCACCGAGCCCAAGAAGGACAAGAAGAAGAAGG | |
| | CCGACGAGACCCAGGCCCTGCCCCAGAGGCAGAAGAAGCAGCAGACCGTGA | |
| | CCCTGCTGCCCGCCGCCGACCTGGACGACTTCAGCAAGCAGCTGCAGCAGA | |
| | GCATGAGCAGCGCCGACAGCACCCAGGCCTGATGA | |

In some embodiments, the vaccine compositions described herein are used to prevent a coronavirus disease in a subject. In other embodiments, the vaccine compositions described herein are used to prevent a coronavirus infection prophylactically in a subject. In further embodiments, the vaccine compositions described herein elicits an immune response in a subject.

Additional sequences and details about methods to select sequences can be found in U.S. Application No. PCT/US21/27355, U.S. Application No. PCT/US21/27340, U.S. Application No. PCT/US21/27341 the specifications of which are hereby incorporated in their entirety by reference.

### EXAMPLES

The following is a non-limiting example of the present invention. It is to be understood that said example is not intended to limit the present invention in any way. Equivalents or substitutes are within the scope of the present invention.

### Example 1: Genome-Wide B Cell, CD4+, and CD8+ T Cell Epitopes that are Highly Conserved Between Human and Animal Coronaviruses, Identified from SARS-CoV-2 as Targets for Preemptive Pan-Coronavirus Vaccines

Using several immunoinformatics and sequence alignment approaches, several human B-cell and CD4+ and CD8+ T cell epitopes were identified that are highly conserved in 1) greater than 81,000 SARS-CoV-2 genome sequences identified in 190 countries on six continents; 2) six circulating CoVs that caused previous human outbreaks of the common cold; 3) nine SL-CoVs isolated from bats; 4) nine SL-CoV isolated from pangolins; 5) three SL-CoVs isolated from civet cats; and 6) four MERS strains isolated from camels. Furthermore, the identified epitopes: 1) recalled B-cells and CD4+ and CD8+ T cells from both COVID-19 patients and healthy individuals who were never exposed to SARS-CoV-2, and 2) induced strong B-cell and T-cell responses in humanized HLA-DR1/HLA-A*02:01 double-transgenic mice. The findings pave the way to develop a preemptive multi epitope pan-coronavirus vaccine (as shown below) to protect against past, current, and future outbreaks.

Rapid evolution of emerging SARS-CoV-2 Variants of Concerns and their genetic similarity with other human and animal coronaviruses: Since the SARS-CoV-2 was first reported in December 2019; the strain has undergone a tremendous degree of evolution. Within 17 months since has been first reported, nearly 593 variants of SARS-CoV-2 have emerged in different parts of the world. Based on their degree of pathogenicity and transmissibility, these variants have been broadly classified into Variants of Interest (VOI) and Variants of Concern (VOC).

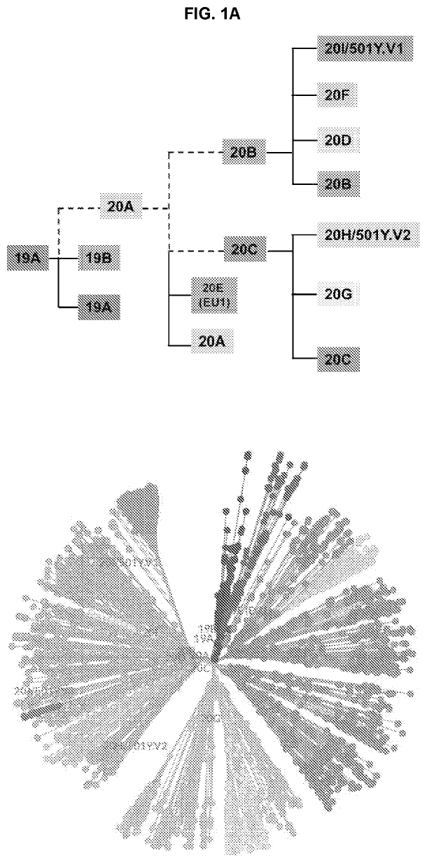

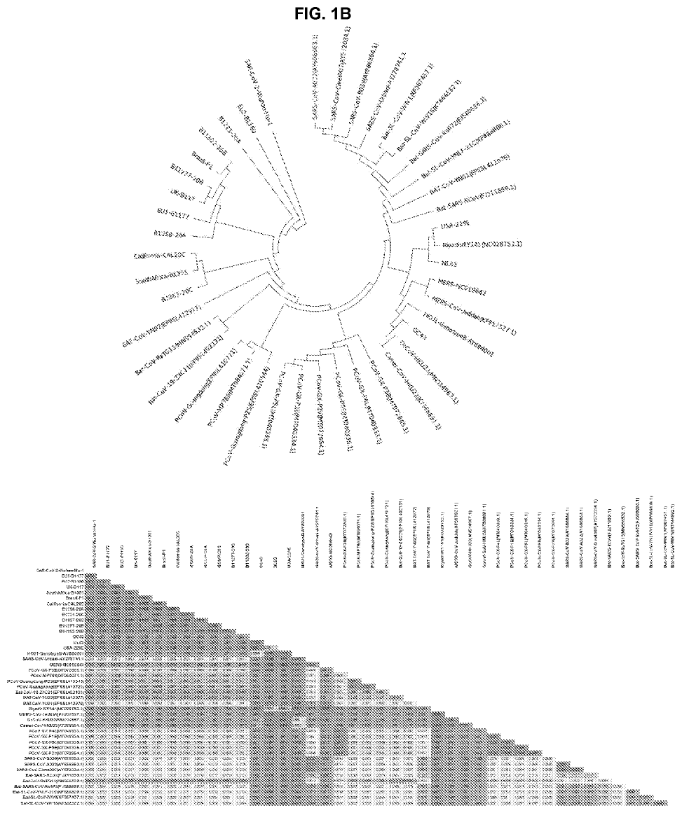

As of Apr. 1, 2021 nearly 712,000 SARS-CoV-2 genome sequences have been reported to the GISAID database. These genome sequences were classified into broad lineages 19A, 19B, 20A, 20B, 20C, 20D, 20E (EU1), 20F, 20G, and 20H based on their degree of relatedness and genetic similarity (FIG. 1A). Currently, two broad nomenclature systems namely PANGO and Nextstrain are in use to categorize the genome sequences that are being reported on daily basis from different parts of the world. Based on the association of the variants with a degree of transmissibility, mortality, increased hospital admission, decreased efficiency of the Covid-19 vaccines towards generating neutralizing antibodies, 17 variants from the larger pool of 593 variants have been classified as VOCs. These VOCs have been reported for the first time in Spain (B.1.177), Australia (B.1.160), England (B.1.1.7), South Africa (B.1.351), Brazil (P.1), California (B.1.427/B.1.429), Scotland (B.1.258), Belgium/Netherlands (B.1.221), Norway/France (B.1.367), Norway/Denmark.UK (B.1.1.277), Sweden (B.1.1.302), North America, Europe, Asia, Africa, and Australia (B.1.525), New York (B.1.526), USA (Wisconsin and other north-central states) (S: 677H.Robin1), USA (Louisiana and other south-central states) (S: 677P.Pelican), and India (B.1.617.1 and B.1.617.2). Later these VOCs spread to different parts of the globe by human beings due to international travels. These VOCs were identified on the basis of a specific set of synonymous and non-synonymous mutations (Table 8).

TABLE 8

| | | Nextstrain | | |
|---|---|---|---|---|
| | PANGO | ID/Other | Synonymous | |
| Origin | Lineage | name(s) | mutations | Non-synonymous mutations |
| Spain | B.1.177 | 20E (EU1)/ | T445C, C6286T, | S: A222V, ORF10: V30L, N: A220V |
| | | 20A.EU1 | C26801G | |
| Australia | B.1.160 | 20A.EU2 | C4543T, G5629T, | S: S477N, N: M234I, N: A376T, |
| | | | C11497T, | ORF1b: A176S, ORF1b: V767L, |
| | | | T26876C | ORF1b: K1141R, ORF1b: E1184D |
| England | B.1.1.7 | 20I/501Y.V1/ | C241T, C913T, | S: H69-, S: V70-, S: Y144-, S: N501Y, |
| | | VOC | C3037T, C5986T, | S: A570D, S: P681H, ORF1a: S3675-, |
| | | 202012/01 | C14676T, | ORF1a: G3676-, ORF1a: F3677-, |
| | | | C15279T, | N: R203K, N: G204R, ORF8: Q27* |
| | | | T16176C | |
| South Africa | B.1.351 | 20H/501Y.V2/ | G174T, C241T, | S: D80A, S: D215G, S: L241-, |
| | | 501Y.V2 | C3037T, C28253T | S: L242-S: A243-, S: K417N, S: E484K, |
| | | | | S: N501Y, S: A701V. |
| | | | | ORF1a: S3675-, ORF1a: G3676-, |
| | | | | ORF1a: F3677-, N: T2051, E: P71L |
| Brazil | P.1 | 20J/501Y.V3 | C241T, T733C, | S: L18F, S: K417T, S: E484K, S: N501Y, |
| | | | C2749T, C3037T, | S: H655Y, |
| | | | A6319G, A6613G, | ORF1a: S3675-, ORF1a: G3676-, |
| | | | C12778T, | ORF1a: F3677- |
| | | | C13860T, | N: P80R, N: R203K, N: G204R |
| | | | A28877T, | |
| | | | G28878C | |
| California | B.1.427 | 20C/S: 452R/ | C2395T, T2597C, | S: S131, S: W152C, S: L452R |
| | B.1.429 | CAL.20C | T24349C, | ORF1a: I4205V, ORF1b: D1183Y |
| | | | G27890T, | |
| | | | A28272T | |
| Scotland | B.1.258 | 20A/S: 439K | C8047T | S: S439K, ORF1a: I2501T |
| Belgium/ | B.1.221 | 20A/S: 98F | C28651T | S: S98F, N: P199L |
| Netherlands | | | | ORF3a: Q38R, ORF3a: G172R, |
| | | | | ORF3a: V202L |
| Norway/ | B.1.367 | 20C/S: 80Y | G4960T, C6070T, | S: D80Y, N: S186Y, N: D377Y |
| France | | | C7303T, C7564T, | ORF1a: T9451, ORF1a: T15671, |
| | | | C10279T, | ORF1a: Q3346K |
| | | | C10525T, | ORF1a: V3475F, ORF1a: M38621 |
| | | | C10582T, | ORF1b: P255T, ORF7a: R80I |
| | | | C27804T | |
| Norway/ | B.1.1.277 | 20B/S: 626S | | S: A626S |
| Denmark. | | | | |
| UK | | | | |
| Sweden | B.1.1.302 | 20B/S: 1122L | | S: V1122L |
| North | B.1.525 | 20A/S: 484K | C241T, C1498T, | S: A67V, S: H69-, S: V70-, S: Y144-, |
| America, | | | A1807G, G2659A, | S: E484K, S: Q677H, S: F888L |
| Europe, | | | C3037T, T8593C, | N: S2-, N: D3Y, N: A12G |
| Asia, Africa, | | | C9593T, C18171T, | ORF1a: S3675-, ORF1a: G3676-, |
| and | | | A20724G, | ORF1a: F3677- |
| Australia | | | C24748T, | |
| | | | A28699G, | |
| | | | G29543T | |
| New York | B.1.526 | 20C/S: 484K | C241T, C3037T, | S: L5F, S: T95I, S: D253G, S: E484K, |
| | | | A20262G, | S: A701V, ORF1b: Q1011H, |
| | | | A28271- | ORF3a: P42L, ORF1a: S3675-, |
| | | | | ORF1a: G3676-, ORF1a: F3677-, |
| | | | | N: P199L, N: M2341, ORF8: T11I |
| USA | | S: 677H.Robin1 | C241T, G1942T, | S: Q677H, N: P67S, N: P199L, N: D377Y |
| (Wisconsin | | | C3037T, A9085G, | ORF1a: T2651, ORF1a: L3352F, |
| and other | | | C14805T | ORF1b: N1653D, ORF1b: R2613C |
| north-central | | | | |
| states) | | | | |
| USA | | S: 677P.Pelican | C241T, C3037T, | S: Q677P, N: P67S, N: P199L |
| (Louisiana | | | C21811A, | ORF1a: T2651, ORF1a: L3352F, |
| and other | | | T29194C, | ORF1b: N1653D, ORF1b: R2613C |
| south-central | | | T29377A | |
| states) | | | | |
| India | B.1.617.1 | 20A/S: 154K | G210T, C241T, | S: E154K, S: L452R, S: E484Q, |
| | | | C3037T, C3457T, | S: D614G, S: P681R, S: Q1071H |
| | | | C26681T, | ORF1b: P314L, ORF1b: G1129C, |
| | | | A28271-, | ORF1b: M13521, ORF1b: K2310R, |
| | | | G29742T | ORF1b: S2312A, N: R203M, N: D377Y, |
| | | | | M: I82S, ORF3a: S26L, ORF1a: T1567I, |
| | | | | ORF1a: T3646A, ORF7a: V82A |
| India | B.1.617.2 | 20A/S: 478K | G210T, C241T, | S: T19R, S: E156-, S: F157-, S: R158G, |
| | | | C3037T, A28271-, | S: L452R, S: T478K, S: D614G, |
| | | | G29742T | S: P681R, S: D950N |
| | | | | ORF1b: P314L, ORF1b: P1000L, |
| | | | | M: I82T, N: D63G, N: R203M, N: D377Y, |
| | | | | ORF3a: S26L, ORF7a: V82A, |
| | | | | ORF7a: T120I |

The evolutionary relationship was screened for between VOCs of human SARS-CoV-2 with SARS-CoV/MERS-CoV strains from previous outbreaks (i.e., Urbani, MERS-CoV, OC43, NL63, 229E, HKU1-genotype-B) and SARS-like Coronaviruses genome sequence (SL-CoVs) obtained from different animal species: Bats (Rhinolophus affinis and Rhinolophus malayanus), civet cats (Paguma larvata) and pangolins (Manis javanica), and MERS-CoVs from camels (Camelus dromedarius and Camelus bactrianus) (FIG. 1B). These sequence alignments revealed similarity of the original human-SARS-CoV-2 strain found in Wuhan, China to the VOCs followed by four bat SL-CoV strains: hCoV-19-bat-Yunnan-RmYN02, bat-CoV-19-ZXC21, and hCoV-19-bat-Yunnan-RaTG13 obtained from the Yunnan and Zhejiang provinces of China (FIG. 1B). With further genetic distance analysis, the least evolutionary divergence was between SARS-CoV-2 isolate Wuhan-Hu-1 and EU2 (B1.160) (0.0001); followed by VOCs CAL20C (0.0003), 20B (B.1.1.302) (0.0003), B.1.1.7 (0.0004), P.1 (0.0005), 20C (0.0005), and B.1.367 (0.0005). Also, SL-CoVs isolate from bats, namely: Bat-CoV-YN02 (0.0511), and Bat-CoV-RaTG13 (0.0402) showed genetic similarity with the SARS-CoV-2 (FIG. 1B), showing an evolutionary convergence of SARS-CoV-2 from the bat strains.

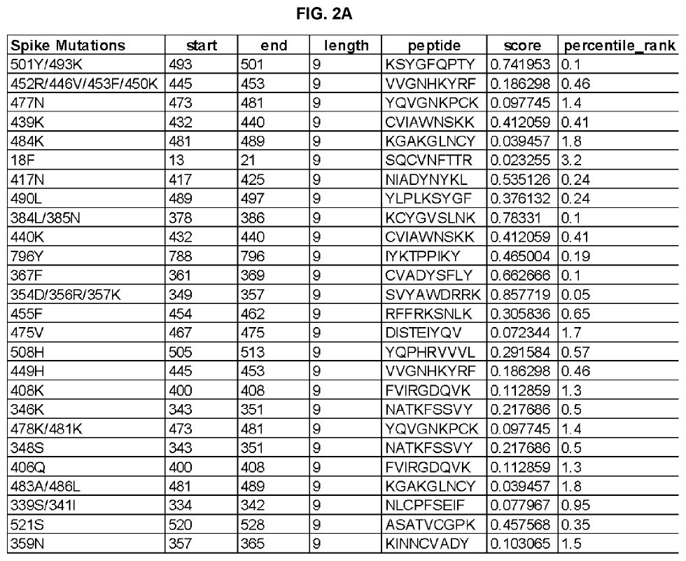

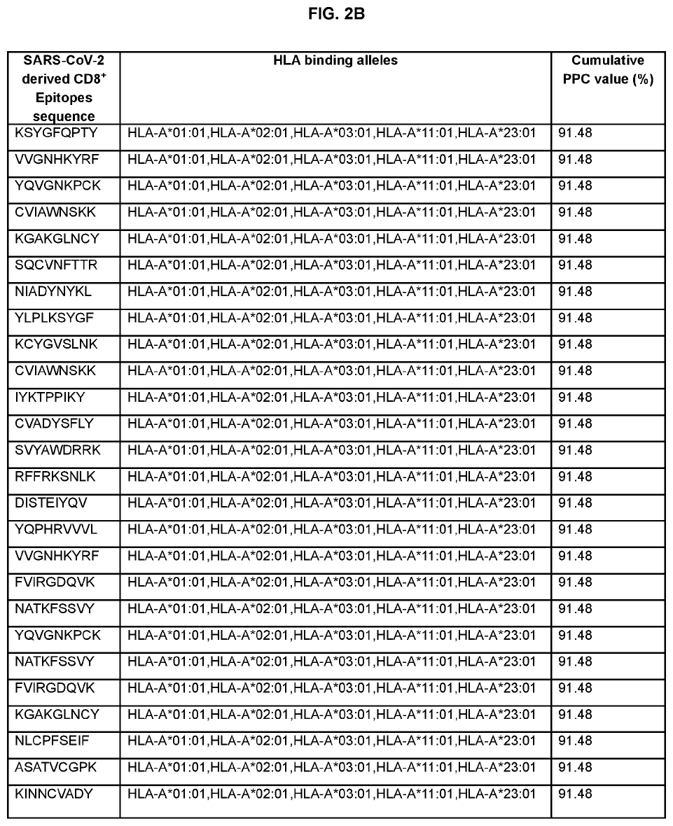

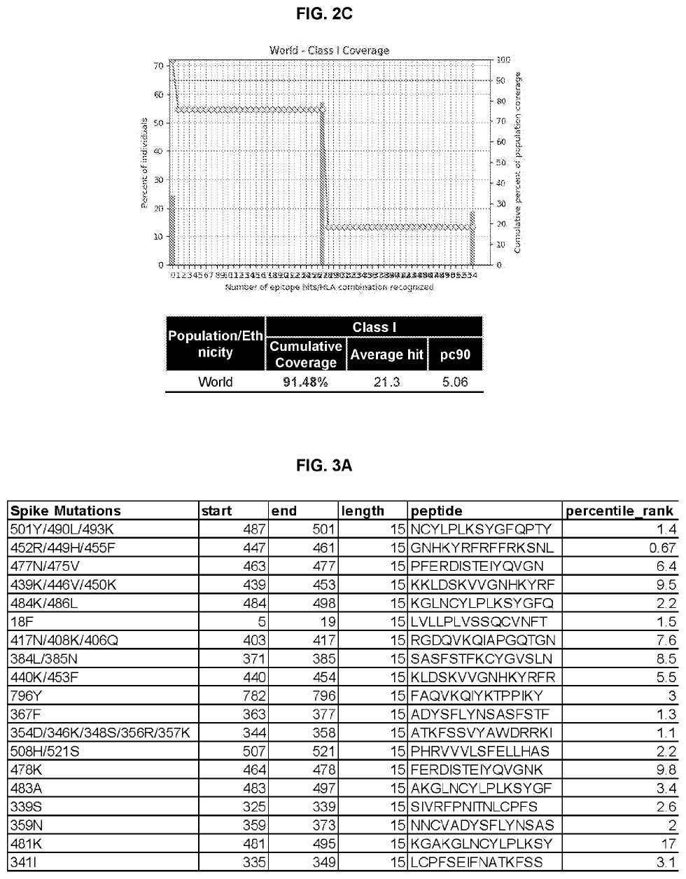

Identification of SARS-CoV-2 CD8⁺ T cell epitopes specific to spike glycoprotein possessing non-synonymous mutations from VOCs: The spike glycoprotein sequence of the SARS-CoV-2-Wuhan-Hu-1 strain (NCBI GenBank accession number MN908947.3) was first substituted with all known non-synonymous mutations reported for the 17 VOCs. Subsequently, this modified spike glycoprotein sequence was used to predict potential CD8⁺ T cell epitopes. For this, multiple databases and algorithms were used, including the SYFPEITHI, MHC-I processing predictions, MHC-I binding predictions, MHC-I immunogenicity and Immune Epitope Database (IEDB). Epitopes restricted to the five most frequent human leukocyte antigen (HLA) class I alleles with 91.48% coverage in worldwide human populations, regardless of race and ethnicity (i.e., HLA-A*01:01, HLA-A*02:01, HLA-A*03:01, HLA-A*11:01, HLA-A*23:01) (63-65) (FIGS. 2A and 2B) were focused on. Using the criteria mentioned above, a total of 26 potential CD8+ T cell epitopes specific to the spike glycoprotein region possessing all the 36 non-synonymous mutations were identified.

These 26 epitopes were subsequently screened for their binding affinity with ACE2 by using molecular docking models across 5 major HLA-A*01:01, HLA-A*02:01, HLA-A*03:01, HLA-A*11:01, HLA-A*23:01 haplotypes (Table 7). The highest binding affinity to HLA-A*02:01 molecules, with the highest interaction similarity (Sᵢₙₜₑᵣ) scores, were recorded for epitope S₂₈₃₋₂₉₁ (KINNCVADY; SEQ ID NO: 137) a Sᵢₙₜₑᵣ score of 179. Whereas minimum Sᵢₙₜₑᵣ score of 108 was observed for epitope S₅₆₂₋₅₇₀ (YQVGNKPCK; SEQ ID NO: 120) (Table 7).

TABLE 7

| | | | | | |
|---|---|---|---|---|---|
| | SARS-CoV-2 | | Protein | | |
| SEQ | derived T-cell | | structure | Estimated | Interaction |
| ID NO: | Epitopes sequence | Protein | similarity | accuracy | similarity |

SARS-CoV-2 derived CD8⁺ T cell epitopes

| | | | | | |
|---|---|---|---|---|---|
| 118 | KSYGFQPTY | Spike | 0.989 | 0.91 | 133 |
| | | glycoprotein | | | |
| 119 | VVGNHKYRF | Spike | 0.984 | 0.93 | 138 |
| | | glycoprotein | | | |
| 120 | YQVGNKPCK | Spike | 0.983 | 0.89 | 174 |
| | | glycoprotein | | | |
| 121 | CVIAWNSKK | Spike | 0.972 | 0.94 | 186 |
| | | glycoprotein | | | |
| 122 | KGAKGLNCY | Spike | 0.967 | 0.92 | 132 |
| | | glycoprotein | | | |
| 123 | SQCVNFTTR | Spike | 0.961 | 0.90 | 170 |
| | | glycoprotein | | | |
| 124 | NIADYNYKL | Spike | 0.978 | 0.86 | 132 |
| | | glycoprotein | | | |
| 125 | YLPLKSYGF | Spike | 0.981 | 0.92 | 135 |
| | | glycoprotein | | | |
| 126 | KCYGVSLNK | Spike | 0.987 | 0.87 | 162 |
| | | glycoprotein | | | |
| 121 | CVIAWNSKK | Spike | 0.991 | 0.85 | 128 |
| | | glycoprotein | | | |
| 127 | IYKTPPIKY | Spike | 0.952 | 0.89 | 140 |
| | | glycoprotein | | | |
| 128 | CVADYSFLY | Spike | 0.959 | 0.93 | 141 |
| | | glycoprotein | | | |
| 129 | SVYAWDRRK | Spike | 0.956 | 0.90 | 141 |
| | | glycoprotein | | | |
| 130 | RFFRKSNLK | Spike | 0.962 | 0.92 | 145 |
| | | glycoprotein | | | |
| 131 | DISTEIYQV | Spike | 0.966 | 0.91 | 189 |
| | | glycoprotein | | | |
| 132 | YQPHRVVVL | Spike | 0.969 | 0.85 | 192 |
| | | glycoprotein | | | |
| 119 | VVGNHKYRF | Spike | 0.964 | 0.92 | 132 |
| | | glycoprotein | | | |
| 133 | FVIRGDQVK | Spike | 0.966 | 0.93 | 167 |
| | | glycoprotein | | | |
| 134 | NATKFSSVY | Spike | 0.972 | 0.96 | 181 |
| | | glycoprotein | | | |
| 120 | YQVGNKPCK | Spike | 0.968 | 0.91 | 108 |
| | | glycoprotein | | | |
| 134 | NATKFSSVY | Spike | 0.971 | 0.91 | 166 |
| | | glycoprotein | | | |
| 133 | FVIRGDQVK | Spike | 0.974 | 0.93 | 152 |
| | | glycoprotein | | | |
| 122 | KGAKGLNCY | Spike | 0.984 | 0.89 | 148 |
| | | glycoprotein | | | |
| 135 | NLCPFSEIF | Spike | 0.989 | 0.87 | 164 |
| | | glycoprotein | | | |
| 136 | ASATVCGPK | Spike | 0.992 | 0.90 | 166 |
| | | glycoprotein | | | |
| 137 | KINNCVADY | Spike | 0.936 | 0.92 | 179 |
| | | glycoprotein | | | |

SARS-CoV-2 derived CD4⁺ T cell epitopes

| | | | | | |
|---|---|---|---|---|---|
| 138 | NCYLPLKSYGFQPTY | Spike | 0.945 | 0.94 | 225 |
| | | glycoprotein | | | |
| 139 | GNHKYRFRFFRKSNL | Spike | 0.987 | 0.95 | 234 |
| | | glycoprotein | | | |
| 140 | PFERDISTEIYQVGN | Spike | 0.993 | 0.92 | 238 |
| | | glycoprotein | | | |
| 141 | KKLDSKVVGNHKYRF | Spike | 0.921 | 0.93 | 256 |
| | | glycoprotein | | | |
| 142 | KGLNCYLPLKSYGFQ | Spike | 0.990 | 0.92 | 272 |
| | | glycoprotein | | | |
| 143 | LVLLPLVSSQCVNFT | Spike | 0.955 | 0.89 | 231 |
| | | glycoprotein | | | |
| 144 | RGDQVKQIAPGQTGN | Spike | 0.959 | 0.91 | 224 |
| | | glycoprotein | | | |
| 145 | SASFSTFKCYGVSLN | Spike | 0.972 | 0.92 | 205 |
| | | glycoprotein | | | |
| 146 | KLDSKVVGNHKYRFR | Spike | 0.970 | 0.90 | 260 |
| | | glycoprotein | | | |
| 147 | FAQVKQIYKTPPIKY | Spike | 0.962 | 0.89 | 242 |
| | | glycoprotein | | | |
| 148 | ADYSFLYNSASFSTF | Spike | 0.963 | 0.96 | 224 |
| | | glycoprotein | | | |
| 149 | ATKFSSVYAWDRRKI | Spike | 0.992 | 0.92 | 249 |
| | | glycoprotein | | | |
| 150 | PHRVVVLSFELLHAS | Spike | 0.987 | 0.92 | 218 |
| | | glycoprotein | | | |
| 151 | FERDISTEIYQVGNK | Spike | 0.952 | 0.91 | 223 |
| | | glycoprotein | | | |
| 152 | AKGLNCYLPLKSYGF | Spike | 0.958 | 0.90 | 204 |
| | | glycoprotein | | | |
| 153 | SIVRFPNITNLCPFS | Spike | 0.967 | 0.87 | 192 |
| | | glycoprotein | | | |
| 154 | NNCVADYSFLYNSAS | Spike | 0.987 | 0.89 | 259 |
| | | glycoprotein | | | |
| 155 | KGAKGLNCYLPLKSY | Spike | 0.982 | 0.91 | 215 |
| | | glycoprotein | | | |
| 156 | LCPFSEIFNATKFSS | Spike | 0.992 | 0.94 | 210 |
| | | glycoprotein | | | |

Altogether, 26 highly potential human CD8+ T cell epitopes were identified possessing the mutations found on VOCs from the modified spike glycoprotein sequence of SARS-CoV-2 possessing all the 36 non-synonymous mutations. If included in a multi-epitope vaccine construct, these epitopes will be able to elicit an immune response and act as immunodominant antigens that are targeted by human CD8+ T cells from both COVID-19 patients infected with any SARS-CoV-2 VOCs.

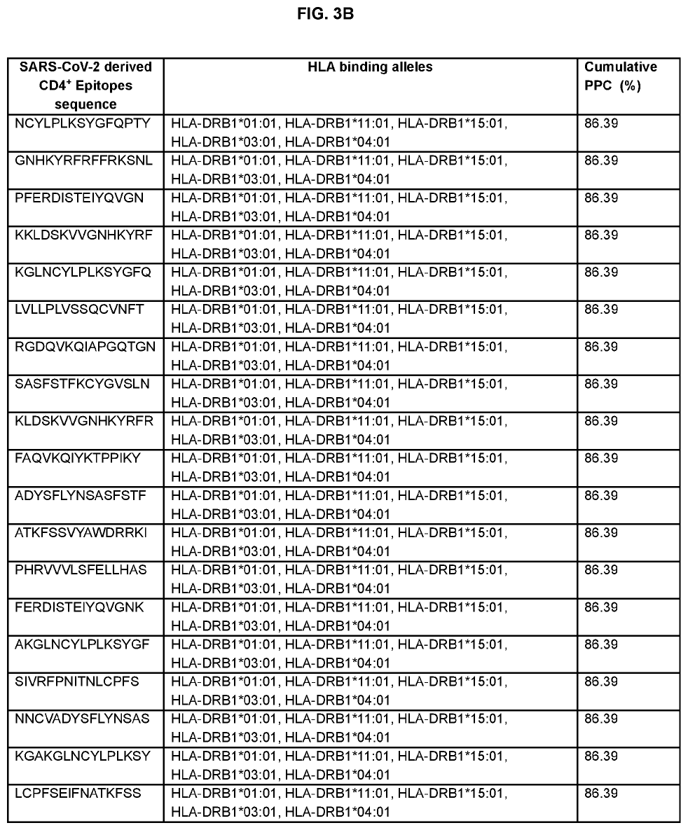

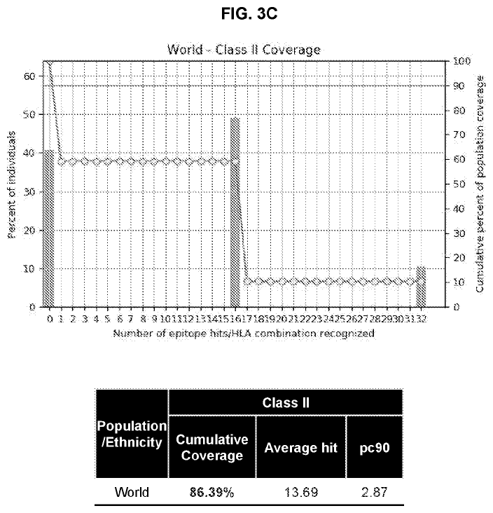

Screening of SARS-CoV-2 CD4+ T cell epitopes specific to spike glycoprotein possessing non-synonymous mutations from VOCs: Subsequently, a total of 19 potential HLA-DR-restricted CD4⁺ T cell epitopes were identified from the spike glycoprotein sequence of SARS-CoV-2-Wuhan-Hu-1 strain (MN908947.3), which was substituted with all the known non-synonymous mutations related to the 17 VOCs. Epitopes were screened using multiple databases and algorithms including the SYFPEITHI, MHC-II Binding Predictions, Tepitool and TEPITOPEpan (FIG. 3A). These potential promiscuous CD4⁺ T cell epitopes were screened in silico against the five most frequent HLA-DR alleles with 86.39% coverage in the human population, regardless of race or ethnicity: HLA-DRB1*01:01, HLA-DRB1*11:01, HLA-DRB1*15:01, HLA-DRB1*03:01, HLA-DRB1*04:01 (FIG. 3A).

Altogether, these results identified 19 potential CD4+ T cell epitopes from the spike glycoprotein region of SARS-CoV-2 substituted with VOC-specific mutations. The highest binding affinity to HLA-DR molecules, with the highest interaction similarity (Sᵢₙₜₑᵣ) scores, was recorded for epitope S₄₄₈₋₄₈₂ (KGLNCYLPLKSYGFQ; SEQ ID NO: 142) with a Sᵢₙₜₑᵣ Score of 272. Whereas minimum Sᵢₙₜₑᵣ score of 192 was observed for epitope S₇₈₅₋₈₀₀ (SIVRFPNITNLCPFS; SEQ ID NO: 153) (Table 7).

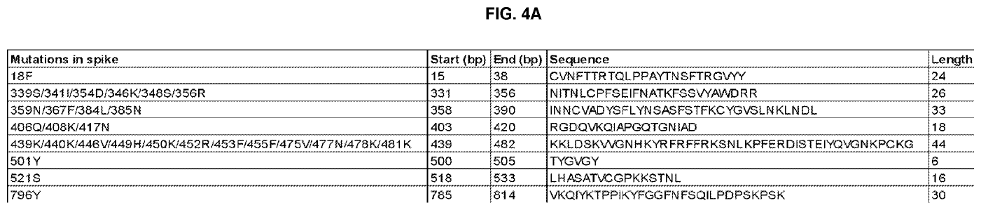

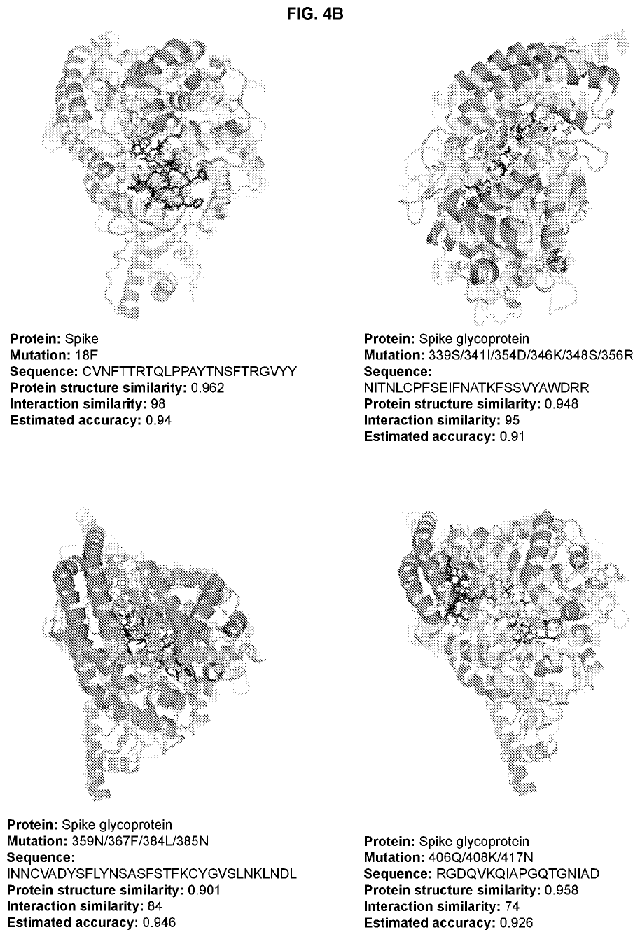

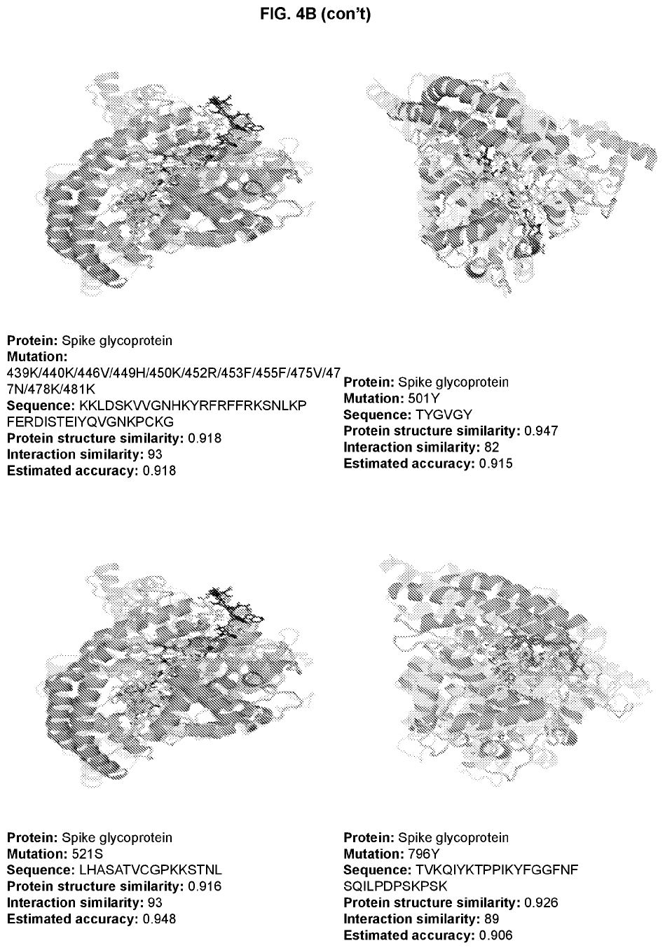

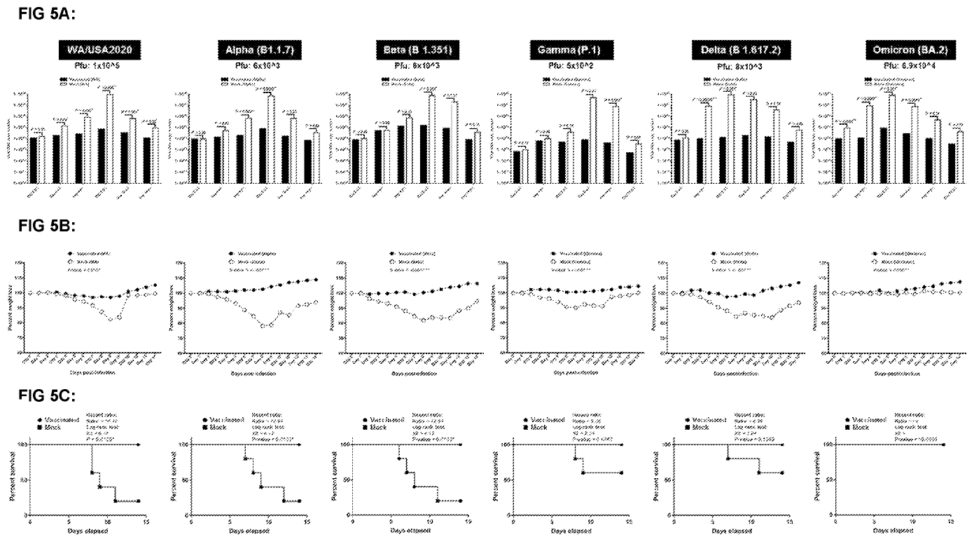

Identification of B-cell epitopes from SARS-CoV-2 Spike protein representing variants of concern specific mutations: Subsequently, potential linear B-cell (antibody) epitopes were predicted on Spike protein sequence of the first SARS-CoV-2-Wuhan-Hu-1 strain (NCBI GenBank accession number MN908947.3) using BepiPred 2.0, with a cutoff of 0.55 (corresponding to a specificity greater than 0.81 and sensitivity below 0.30) and considering sequences having more than 5 amino acid residues. This stringent screening process initially resulted in the identification of 8 linear B-cell epitopes (FIG. 4A).

Higher interaction similarity scores were observed for epitopes S₃₈₂₋₄₀₅ (CVNFTTRTQLPPAYTNSFTRGVYY; SEQ NO 117) and S₅₇₆₋₆₂₀ (KKLDSKVVGNHKYRFRFFRKSNLKPFERDISTEIYQVGNKPCKG: SEQ ID NO: 157) when molecular docking was performed against the ACE2 receptor (FIG. 4B). S₃₈₂₋₄₀₅ possesses mutation 18F, whereas S₅₇₆₋₆₂₅ possesses mutations 439K/440K/446V/449H/450K/452R/453F/455F/475V/477N/478K/481K.

Immunization with pool of CD8⁺ T cell, CD4⁺ T cell, and B cell peptides provide protection against all the current SARS-CoV-2 variants of concerns: The inventors have immunized a group of triple transgenic h-ACE2-HLA-A2/DR mice with pool of CD8⁺ T cell, CD4⁺ T cell, and B cell peptides on Day 0 and Day 14. A second group of triple transgenic mice were left non-immunized. Following 14 days of the two doses of immunization, on Day 28 of the experiment mice were challenged with Washington, Alpha, Beta, Gamma, Delta, and Omicron variants of SARS-CoV-2. Both immunized and non-immunized groups of mice challenged with different variants of SARS-CoV-2 were monitored for next 14 days till Day 42 of the experiment for viral titration (FIG. 5A), physical manifestations (percent weight loss (FIG. 5B), survival (FIG. 5C)). The inventors have observed 100% survival in the immunized group of mice irrespective of the SARS-CoV-2 variant (FIG. 5C). This shows the strong immunogenicity provided by the pool of the T cell and B cell peptides; which in turn is providing absolute protection against any known SARS-CoV-2 variants of concern known as of June 2022.

Sequence-based variant effect predictor analysis to access the pathogenicity of mutations in VOCs: Fourteen non-synonymous mutations were found (S:S477N, S:A222V, S:D253G, S:L452R, S:S439K, S:S98F, S:A701V, S:K417N, S:N501Y, S:D215G, S:D614G, S:L18F, S:E484K, S:A67V) being screened by at-least one of the multiple tools used to predict the effect of the variants. Three tools namely GERP++ RS, phastCons and PhyloP were used to evaluate the conservancy score calculation. Further five tools namely SIFT, PolyPhen, Condel, PROVEAN, and Mutation Assessor of variant effect predictor server (VEP) were used to predict the effect of the mutations with the degree of pathogenicity with COVID-19. Two mutations S:K417N, and S:E484K were predicted to be of higher pathogenicity by at least 4 of the 5 tools. Whereas the mutation S:D614G was predicted as pathogenic by all the 5 variant effect predictor tools (FIG. 8). Thus, inclusion of epitopes specific to these mutations in a vaccine will aid in protecting a larger mass of the population who otherwise are prone to hospitalization and mortality when being infected by VOCs Alpha (B.1.1.7), Beta (B.1.351), Gamma (P.1), Delta (B.1.617.1, B.1.617.2), and Omicron (BA.1, BA.2) that possess S:D614G/S:K417N/S:E484K.

Thus, inclusion of epitopes specific to these mutations in a vaccine will aid in protecting a larger mass of the population who otherwise are prone to hospitalization and mortality when being infected by VOCs B.1.617.1, B.1.617.2, B.1.1.7, B.1.351, P.1 that possess S:D614G/S:K417N/S:E484K.

Sequence comparison among variants of SARS-CoV-2 with the common cold and animal CoV strains: 712,000 human SARS-CoV-2 genome sequences were retrieved from GISAID database representing countries from North America, South America, Central America, Europe, Asia, Oceania, Australia and Africa (FIG. 1A-1B). This comprised of variants of concern (VOC)/variants of interest (VOI) of SARS-CoV-2 (B.1.177, B.1.160, B.1.1.7, B.1.351, P.1, B.1.427/B.1.429, B.1.258, B.1.221, B.1.367, B.1.1.277, B.1.1.302, B.1.525, B.1.526, S: 677H.Robin1, S: 677P.Pelican, B.1.617.1, B.1.617.2) and common cold SARS-CoV strains (SARS-CoV-2-Wuhan-Hu-1 (MN908947.3), SARS-CoV-Urbani (AY278741.1), HKU1-Genotype B (AY884001), CoV-OC43 (KF923903), CoV-NL63 (NC_005831), CoV-229E (KY983587)) and MERS (NC_019843)). Also, for evaluating the evolutionary relationship among the SARS-CoV-2 variants and common cold CoV strains, whole-genome sequences from bat ((RATG13 (MN996532.2), ZXC21 (MG772934.1), YN01 (EPI_ISL_412976), YN02 (EPI_ISL_412977), WIV16 (KT444582.1), WIV1 (KF367457.1), YNLF_31C (KP886808.1), Rs672 (FJ588686.1)), pangolin (GX-P2V (MT072864.1), GX-P5E (MT040336.1), GX-P5L (MT040335.1), GX-P1E (MT040334.1), GX-P4L (MT040333.1), GX-P3B (MT072865.1), MP789 (MT121216.1), Guangdong-P2S (EPI_ISL_410544)), camel (KT368891.1, MN514967.1, KF917527.1, NC_028752.1), and civet (Civet007, A022, B039)) were included. All the sequences included in this study were retrieved either from the NCBI GenBank or GISAID. Multiple sequence alignment was performed keeping SARS-CoV-2-Wuhan-Hu-1 (MN908947.3) protein sequence as a reference against all the SARS-CoV-2 VOCs, common cold, and animal CoV strains. The sequences were aligned using ClustalW algorithm in MEGA X.

SARS-CoV-2 CD8+ and CD4+ T Cell Epitope Prediction: Epitope prediction was performed considering the spike glycoprotein (YP_009724390.1) for the reference SARS-CoV-2 isolate, Wuhan-Hu-1. The reference spike protein sequence was substituted with all the non-synonymous mutations reported on the 17 VOCs to screen CD8⁺ T cell (FIG. 2A) and CD4⁺ T cell (FIG. 3A) epitopes. The tools used for CD8+ T cell-based epitope prediction were SYFPEITHI, MHC-I binding predictions, and Class I Immunogenicity. Of these, the latter two were hosted on the IEDB platform. Multiple databases and algorithms were used for the prediction of CD4⁺ T cell epitopes, namely SYFPEITHI, MHC-II Binding Predictions, Tepitool, and TEPITOPEpan. For CD8⁺ T cell epitope prediction, the 5 most frequent HLA-A class I alleles (HLA-A*01:01, HLA-A*02:01, HLA-A*03:01, HLA-A*11:01, HLA-A*23:01) with nearly 91.48% coverage of the world population, regardless of race and ethnicity (FIGS. 2B and 2C) were selected, using a phenotypic frequency cutoff≥6%. Similarly, for CD4+ T cell epitope prediction, selected HLA-DRB1*01:01, HLA-DRB1*11:01, HLA-DRB1*15:01, HLA-DRB1*03:01, HLA-DRB1*04:01 alleles with population coverage of 86.39% (FIGS. 3B and 3C). Subsequently, using NetMHC the SARS-CoV-2 protein sequence was analyzed against all the aforementioned MHC-I and MHC-II alleles. Epitopes with 9mer length for MHC-I and 15mer length for MHC-II were predicted. Subsequently, the peptides were analyzed for binding stability to the respective HLA allotype. The stringent epitope selection criteria were based on picking the top 1% epitopes focused on prediction percentile scores. N and O glycosylation sites were screened using NetNGlyc 1.0 and NetOGlyc 4.0 prediction servers, respectively.

SARS-CoV-2 B Cell Epitope Prediction: Linear B cell epitope predictions were carried out on the spike glycoprotein(S), the primary target of B cell immune responses for SARS-CoV. The spike glycoprotein sequence was substituted with non-synonymous mutations reported on 17 VOCs. The BepiPred 2.0 algorithm embedded in the B cell prediction analysis tool hosted on IEDB platform was used. For each protein, the epitope probability score for each amino acid and the probability of exposure was retrieved. Potential B cell epitopes were predicted using a cutoff of 0.55 (corresponding to a specificity greater than 0.81 and sensitivity below 0.3) and considering sequences having more than 5 amino acid residues. This screening process resulted in 8 B-cell peptides (FIG. 4A). These epitopes represent all the major non-synonymous mutations reported among the VOCs. One B-cell epitope (S₄₃₉₋₄₈₂) was observed to possess the maximum number of VOC-specific mutations. Structure-based antibody prediction was performed by using Discotope 2.0, and a positivity cutoff greater than-2.5 was applied (corresponding to specificity greater than or equal to 0.80 and sensitivity below 0.39), using the SARS-CoV-2 spike glycoprotein structure (PDB ID: 6M1D), which was further substituted with non-synonymous mutations.

Population-Coverage-Based T Cell Epitope Selection: For a robust epitope screening, the conservancy of CD8⁺ T cell, CD4⁺ T cell, and B cell epitopes within spike glycoprotein of Human-SARS-CoV-2 genome sequences representing North America, South America, Africa, Europe, Asia, and Australia was evaluated. As of May 17, 2021, the GISAID database recorded 1,581,990 human-SARS-CoV-2 genome sequences and the number of genome sequences continues to grow daily. In the present analysis, 712,000 human-SARS-CoV-2 genome sequences representing six continents were extrapolated from the GISAID database as on Apr. 1, 2021. Population coverage calculation (PPC) was carried out using the Population Coverage software hosted on IEDB platform. PPC was performed to evaluate the distribution of screened CD8⁺ and CD4⁺ T cell epitopes in world population at large in combination with HLA-I (HLA-A*01:01,HLA-A*02:01,HLA-A*03:01, HLA-A*11:01, HLA-A*23:01), and HLA-II (HLA-DRB1*01:01, HLA-DRB1*11:01, HLA-DRB1*15:01, HLA-DRB1*03:01, HLA-DRB1*04:01) alleles.

### Example 2: Cross-Protection Induced by Highly Conserved Human B, CD4+, and CD8+ T Cell Epitopes-Based Coronavirus Vaccine Against Severe Infection, Disease, and Death Caused by Multiple SARS-CoV-2 Variants of Concern

The present invention features a multi-epitope-based Coronavirus vaccine that incorporated B, CD4⁺, and CD8⁺ T cell epitopes conserved among all known SARS-CoV-2 VOCs and selectively recognized by CD8⁺ and CD4⁺ T-cells from asymptomatic COVID-19 patients irrespective of VOC infection. The safety, immunogenicity, and cross-protective immunity of this pan-Coronavirus vaccine were studied against six VOCs using an innovative triple transgenic h-ACE-2-HLA-A2/DR mouse model. The Pan-Coronavirus vaccine: (i) is safe: (ii) induces high frequencies of lung-resident functional CD8⁺ and CD4⁺ T_{EM} and T_{RM} cells; and (iii) provides robust protection against virus replication and COVID-19-related lung pathology and death caused by six SARS-CoV-2 VOCs: Alpha (B.1.1.7), Beta (B.1.351), Gamma or P1 (B.1.1.28.1), Delta (lineage B.1.617.2) and Omicron (B.1.1.529).

Highly conserved SARS-CoV-2 epitopes are selectively recognized by CD8⁺ and CD4⁺ T-cells from asymptomatic COVID-19 patients irrespective of variants of concern infection: To identify “universal” SARS-CoV-2 epitopes to be included in a multi-epitope pan-Coronavirus Vaccine; the degree of conservancy for human CD8⁺ T cell, CD4⁺ T cell, and B-cell epitopes that span the whole SARS-CoV-2 genome was screened. CD8⁺ T cell epitopes were screened for their conservancy against variants namely h-CoV-2/Wuhan (MN908947.3), h-CoV-2/WA/USA2020 (OQ294668.1), h-CoV-2/Alpha (B1.1.7) (OL689430.1), h-CoV-2/Beta (B 1.351) (MZ314998), h-CoV-2/Gamma (P.1) (MZ427312.1), h-CoV-2/Delta (B.1.617.2) (OK091006.1), and h-CoV-2/Omicron (B.1.1.529) (OM570283.1) (33). 100% conservancy was observed in all the SARS-CoV-2 variants of concern for 14 of the 16 predicted CD8⁺ T cell epitopes (ORF1ab₂₂₁₀₋₂₂₁₈, ORF1ab₃₀₁₃₋₃₀₂₁; ORF1ab₄₂₈₃₋₄₂₉₁, ORF1ab₆₇₄₉₋₆₇₅₇, ORF6₃₋₁₁, ORF7b_{28 34}, ORF10_{3 11}, ORF10_{5 13}, S₉₅₈₋₉₆₆, S₁₀₀₀₋₁₀₀₈, S₁₂₂₀₋₁₂₂₈, E₂₀₋₂₈, M₅₂₋₈₀, and M₈₉₋₉₇). The only exceptions were epitopes E₂₆₋₃₄ and ORF8a_{73 81} which showed an 88.8% conservancy against Beta (B.1.351) and Alpha (B.1.1.7) variants respectively. All of the 6 highly immunodominant “universal” CD4⁺ T cell epitopes (ORF1a₁₃₅₀₋₁₃₆₅, ORF6₁₂₋₂₆, ORF8b₁₋₁₅, S₁₋₁₃, M₁₇₆₋₁₉₀, and N₃₈₈₋₄₀₃), remained 100% conserved across all the SARS-CoV-2 VOCs.

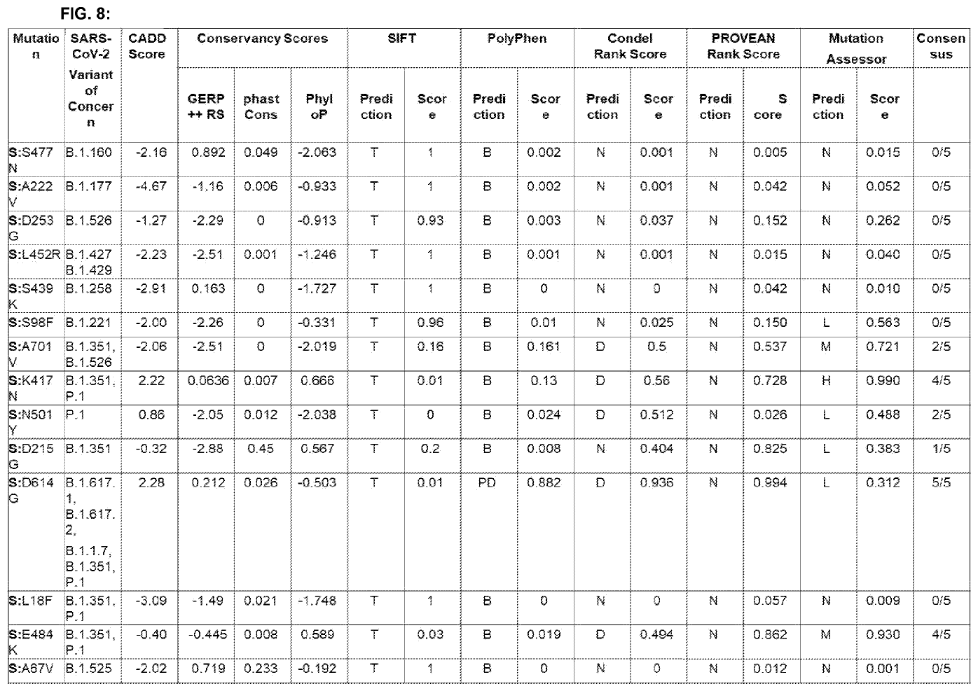

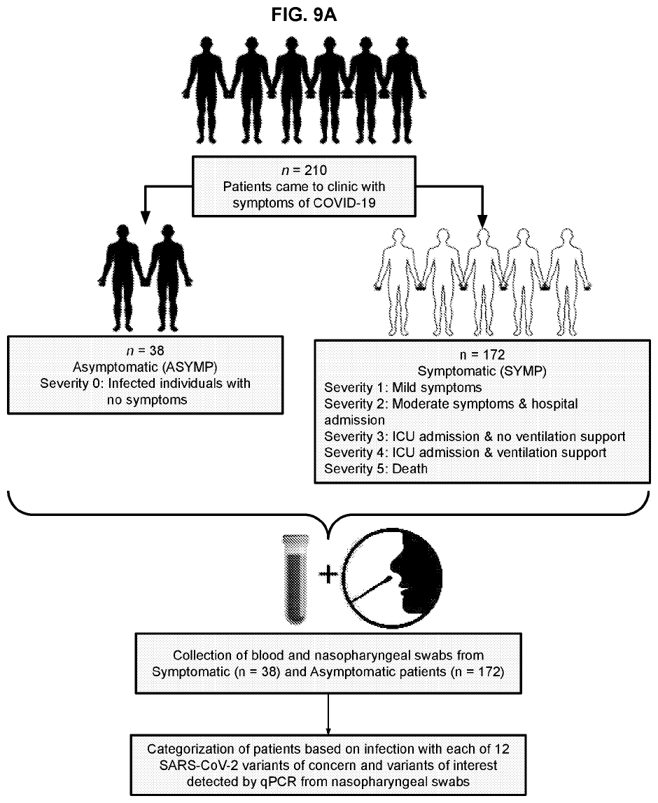

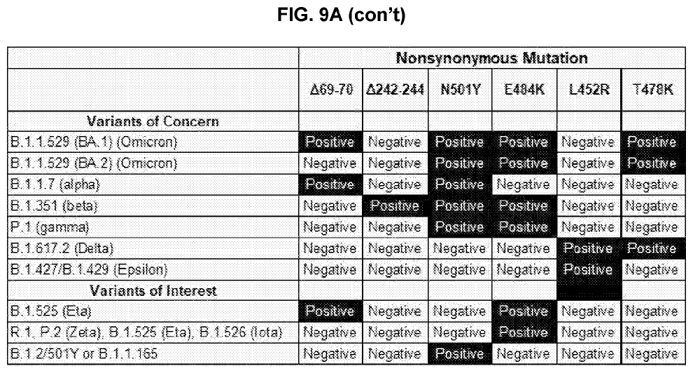

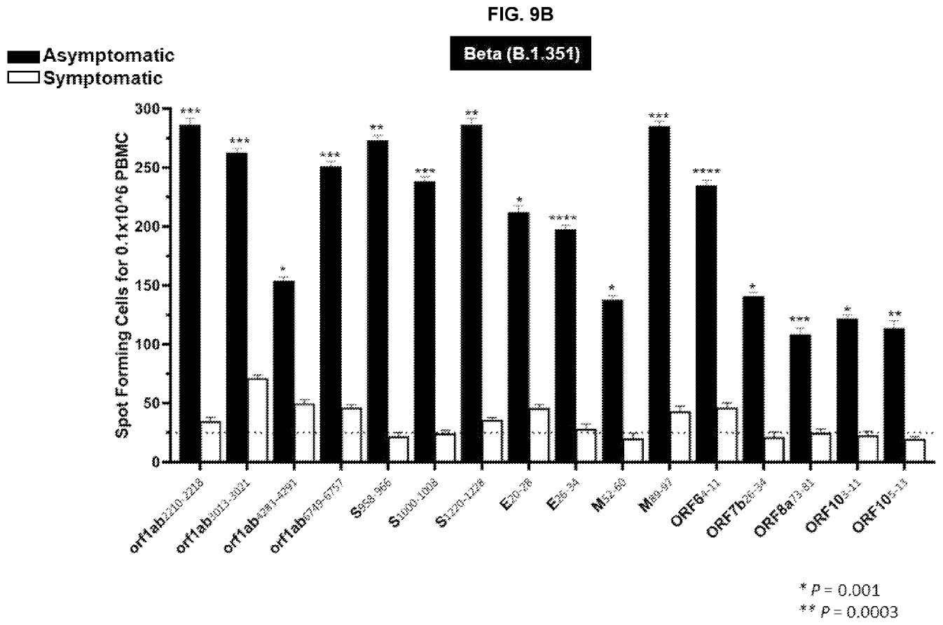

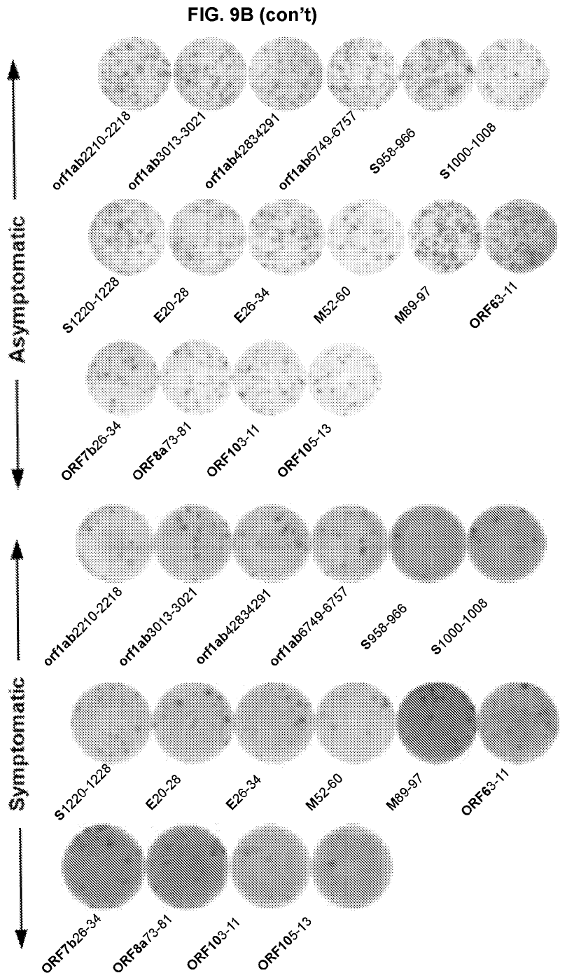

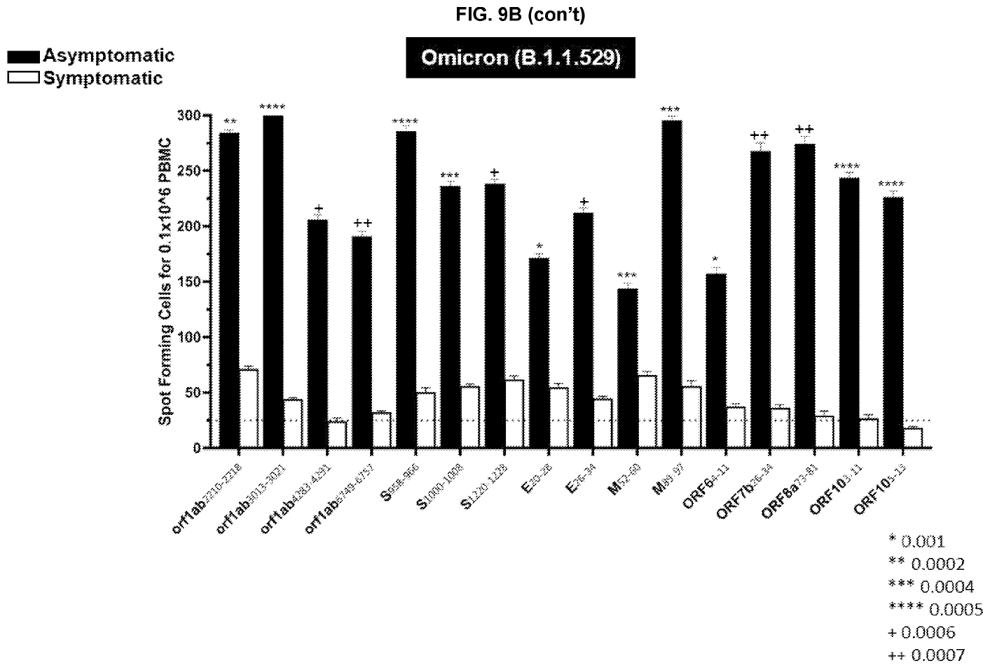

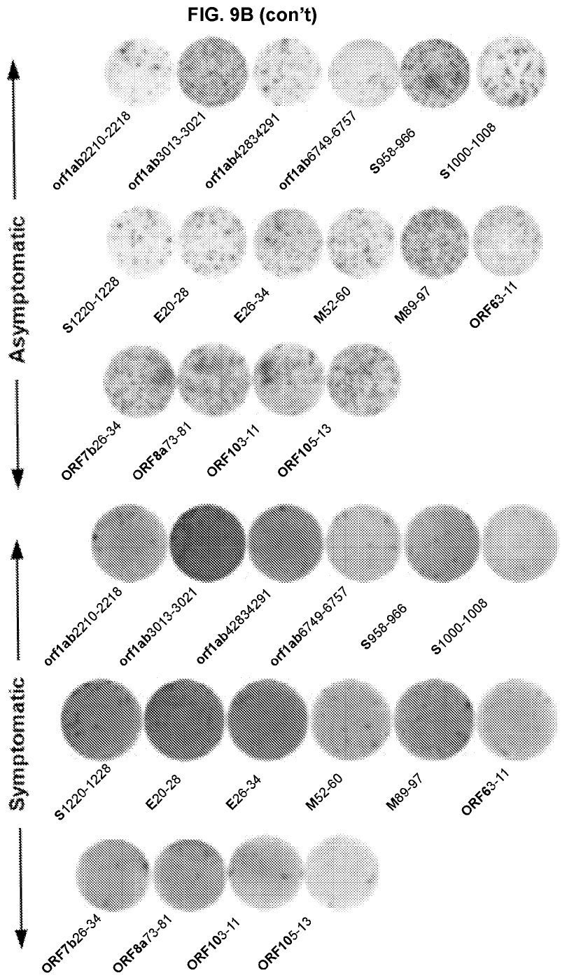

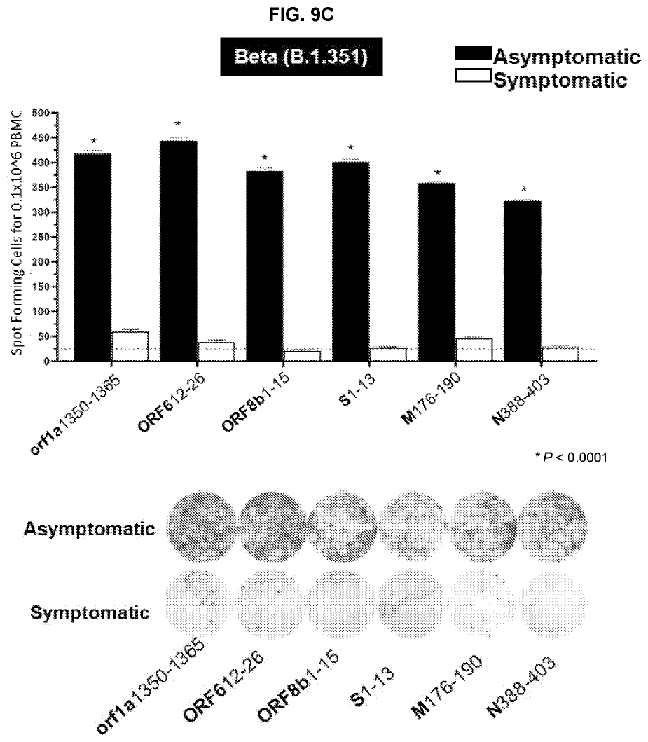

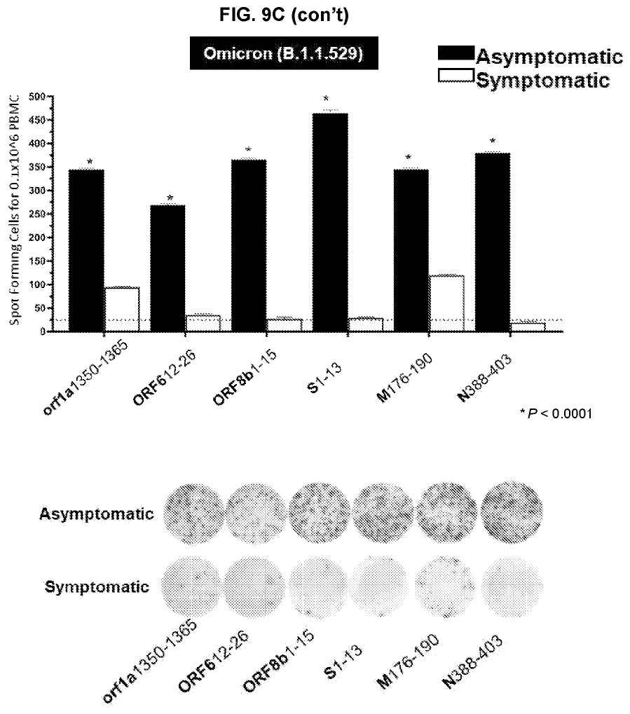

Next, the highly conserved “universal” CD8⁺ and CD4⁺ T cell epitopes were differentially recognized by T cells from asymptomatic (ASYMP) versus symptomatic (SYMP) COVID-19 patients. The magnitude of CD8⁺ and CD4⁺ T cell responses specific to each of the conserved epitopes among 38 ASYMP and 172 SYMP COVID-19 patients were compared. COVID-19 patients infected with SARS-CoV-2 Beta (B.1.351) and SARS-CoV-2 Omicron (B.1.1.529) were recruited spanning two years of the COVID-19 pandemic (FIG. 9A). Fresh PBMCs were isolated from SYMP and ASYMP COVID-19 patients, on average within 4 days after reporting their first symptoms. PBMCs were then stimulated in vitro for 72 hours using each of the 16 CD8⁺ T cell epitopes or each of the 6 CD4⁺ T cell epitopes. Numbers of responding IFN-γ-producing CD8⁺ and CD4⁺ T cells (quantified in ELISpot assay as the number of IFN-γ-spot forming cells, or “SFCs”) were subsequently determined.

ASYMP COVID-19 patients showed significantly higher frequencies of SARS-CoV-2 epitope-specific IFN-γ-producing CD8+ T cells (mean SFCs>25 per 1×10⁶ pulmonary immune cells), irrespective of infection with Beta (P<0.5, FIG. 9B) or Omicron (P<0.5, FIG. 9B) variants. In contrast, severely ill or hospitalized symptomatic COVID-19 patients showed significantly lower frequencies of SARS-CoV-2 epitope-specific IFN-γ-producing CD8+ T cells (P<0.5, FIG. 9B) or Omicron (P<0., FIG. 9B) variants. This observation was consistent regardless of whether CD8+ T cell's targeted epitopes were from structural or non-structural SARS-CoV-2 protein antigens, suggesting that strong CD8+ T cell responses specific to selected “universal” SARS-CoV-2 epitopes were commonly associated with better COVID-19 outcomes. In contrast, low SARS-CoV-2-specific CD8+ T cell responses were more commonly associated with severe onset of disease.

Similarly, higher frequencies of functional IFN-γ-producing CD4⁺ T cells ASYMP COVID-19 patients (mean SFCs>25 per 1×10⁶ pulmonary immune cells), irrespective of infection with Beta (P<0.5, FIG. 9C) or Omicron (P<0.5, FIG. 9C) variants. Whereas reduced frequencies of IFN-γ-producing CD4⁺ T cells were detected in SYMP COVID-19 patients, irrespective of infection with Beta (P<0.5, FIG. 9C) or Omicron (P<0.5, FIG. 9C) variants. This observation was consistent regardless of whether CD4⁺ T cell's targeted epitopes were from structural or non-structural SARS-CoV-2 protein antigens. Our results suggest strong CD4⁺ T cell responses specific to selected “universal” SARS-CoV-2 epitopes were commonly associated with better COVID-19 outcomes. In contrast, low SARS-CoV-2-specific CD4⁺ T cell responses were more commonly associated with severe disease onset.

Altogether these results: (1) demonstrate an important role of SARS-CoV-2-specific CD4⁺ and CD8⁺ T cells directed against highly conserved structural and non-structural SARS-CoV-2 epitopes in protection from severe COVID-19 symptoms, and (2) highlight the potential importance of these highly conserved “asymptomatic” epitopes in mounting protected CD4⁺ and CD8⁺ T cell responses against multiple SARS-CoV-2 VOCs.

A pan-Coronavirus vaccine composed of a mixture of conserved “asymptomatic” CD4⁺ and CD8⁺ T cell epitopes provides robust protection against infection and disease caused by six SARS-CoV-2 variants of concern: A prototype pan-Coronavirus vaccine composed of a mixture of 6 conserved “asymptomatic” CD4⁺ T cell epitopes and 16 conserved “asymptomatic” CD4⁺ and CD8⁺ T cell epitopes was used, previously this vaccine was identified to span the whole SARS-CoV-2 genome. CD4⁺ and CD8⁺ T cell epitopes that show immunodominance selectively in SYMP COVID-19 patients infected with various SARS-CoV-2 VOCs was focused mainly on.

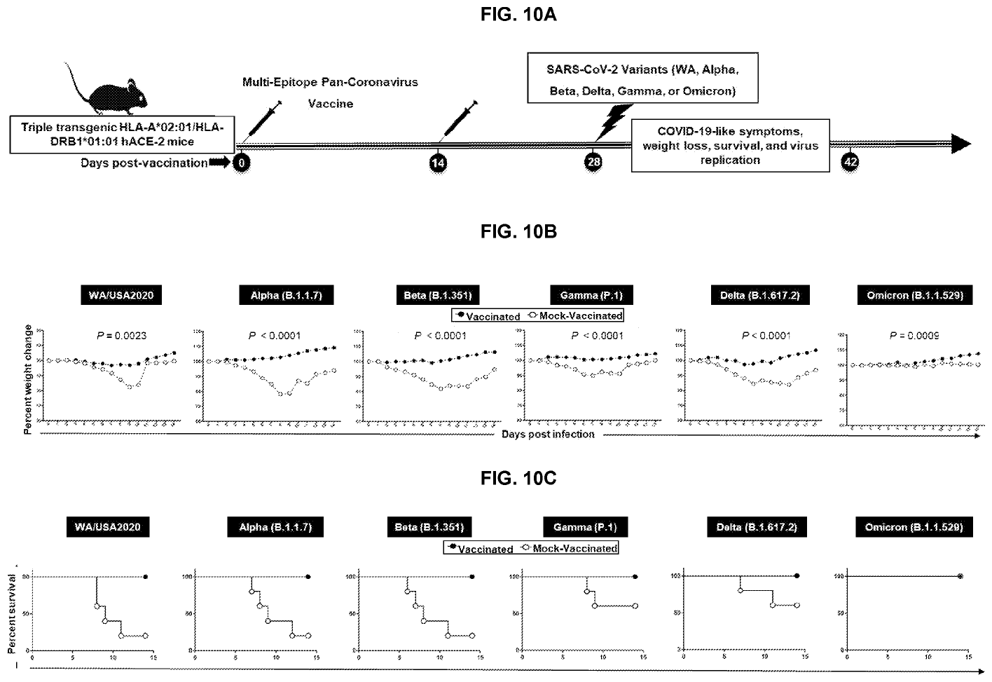

A pool of peptides comprising 25 μg each of 16 CD8+ T cell peptides (ORF1ab₂₂₁₀₋₂₂₁₈, ORF1ab₃₀₁₃₋₃₀₂₁, ORF1ab₄₂₈₃₋₄₂₉₁, ORF1ab₆₇₄₉₋₆₇₅₇, ORF6₃₋₁₁, ORF7b₂₆₋₃₄, ORF8a₇₃₋₈₁, ORF10₃₋₁₁, ORF10₅₋₁₃, S₉₅₈₋₉₈₆, S₁₀₀₀₋₁₀₀₈, S₁₂₂₀₋₁₂₂₈, E₂₀₋₂₆, E₂₆₋₃₄, M₅₂₋₈₀, and M₈₉₋₉₇), 6 CD4+ T cell epitopes (ORF1a₁₃₅₀₋₁₃₆₅, ORF6₁₂₋₂₈, ORF8b₁₋₁₅, S₁₋₁₃, M₁₇₆₋₁₉₀, and N₃₈₈₋₄₀₃). and 7 B-cell peptides selected from the Spike protein, were mixed with cpG1826 adjuvant and administered subcutaneously on Day 0 and Day 14 to 7-8 week old triple transgenic HLA-A*02:01/HLA-DR hACE-2 mice (n=30). The remaining group of the mock-immunized received vehicle alone (n=30) (FIG. 10A). Fourteen days after the second immunization (i.e., day 28) mice were divided into 6 groups and intranasally infected with 1×10⁵ pfu of SARS-CoV-2 (USA-WA1/2020) (n=10), 6×10³ pfu of SARS-CoV-2-Alpha (B.1.1.7) (n=10), 6×10³ pfu of SARS-CoV-2-Beta (B.1.351) (n=10), 5×10² pfu of SARS-CoV-2-Gamma (P.1) (n=10), 8×10³ pfu of SARS-CoV-2-Delta (B.1.617.2) (n=10), and 6.9×10⁴ pfu of SARS-CoV-2-Omicron (B.1.1.529) (n=10) (FIG. 10A).

Mice that received the pan-Coronavirus vaccine showed significant protection from weight loss (FIG. 10B) and death (FIG. 10C) following infection with each of the six SARS-CoV-2 variants of concern: WA/USA2020, Alpha (B.1.1.7), Beta (B.1.351), Gamma (P.1), Delta (B.1.617.2), and Omicron (B.1.1.529). All mice immunized with the conserved pan-Coronavirus vaccine survived infection with SARS-CoV-2 variants of concern. In contrast to mock-immunized mice where 60% mortality was detected among WA/USA2020 infected mice, 80% mortality among Alpha (B.1.1.7) and Beta (B.1.351) infected mice, 40% mortality among Gamma (P.1) and Delta (B.1.617.2) variants infected mice (FIG. 10C). Mortality was not observed for mock-immunized mice infected with the SARS-CoV-2 Omicron (B.1.1.529) variant (FIG. 10C).

Throat swabs were collected from the vaccinated and mock-vaccinated groups of mice on days 2, 4, 6, 8, 10, and 14 post-infection (p.i.) and were processed to detect the viral RNA copy number by qRT-PCR (FIG. 10D). Compared to the viral RNA copy number detected from the mock-vaccinated group of mice, a statistically significant decrease was detected in the viral RNA copy number among vaccinated groups of mice on day 4 p.i. for SARS-CoV-2 WA/USA2020 (P=0.04), Delta (B.1.617.2) (P=0.00009), and Omicron (B.1.1.529) (P=0.007); on day 6 p.i. for SARS-CoV-2 WA/USA2020 (P=0.002), Alpha (B.1.1.7) (P=0.002), Delta (B.1.617.2) (P=0.001), and Omicron (B.1.1.529) (P=0.001); on day 8 p.i. for SARS-CoV-2 WA/USA2020 (P=0.006), Alpha (B.1.1.7) (P=0.0002), Beta (B.1.351) (P=0.002), Gamma (P.1) (P=0.04), and Omicron (B.1.1.529) (P=0.0001); on day 10 p.i. for SARS-CoV-2 WA/USA2020 (P=0.005), Gamma (P.1) (P=0.008); and on day 14 p.i. for SARS-CoV-2 WA/USA2020 (P=0.02) (FIG. 10D). This result suggests that the pan-Coronavirus vaccine showed significant protection from virus replication for most of SARS-CoV-2 variants and confirms a plausible anti-viral effect following immunization with asymptomatic B, CD4+ and CD8+ T cell epitopes carefully selected as being highly conserved from multiple SARS-CoV-2 variants.

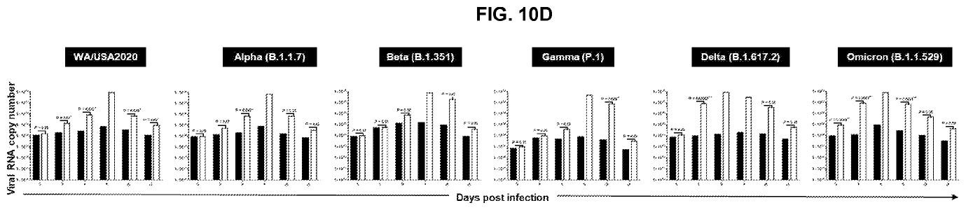

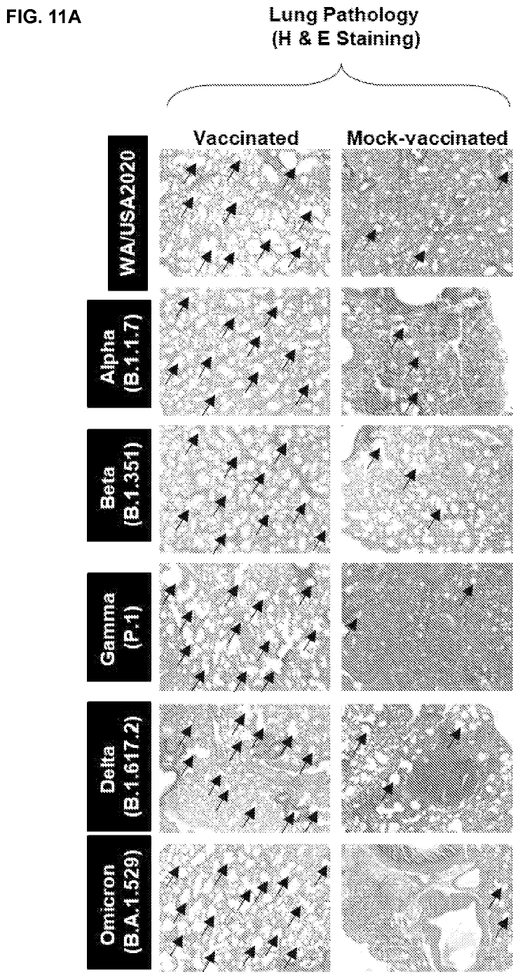

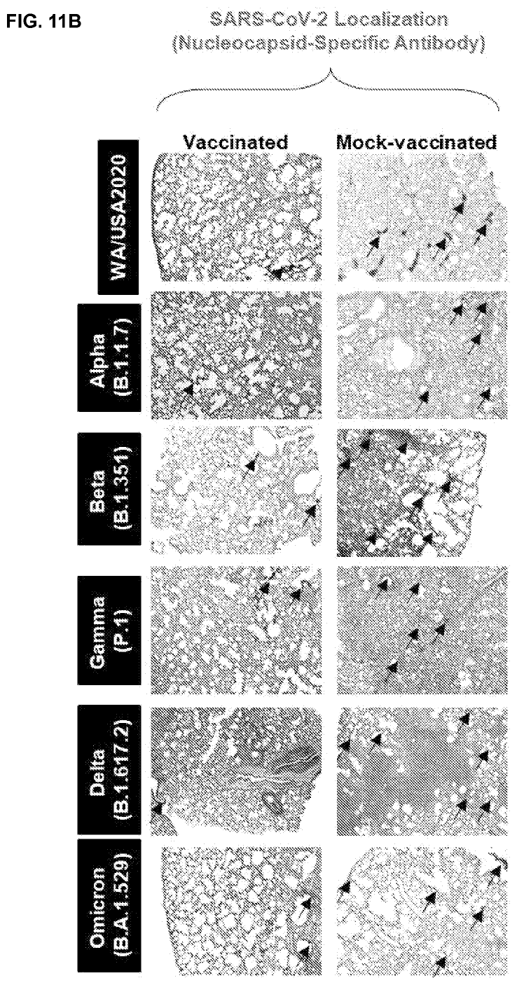

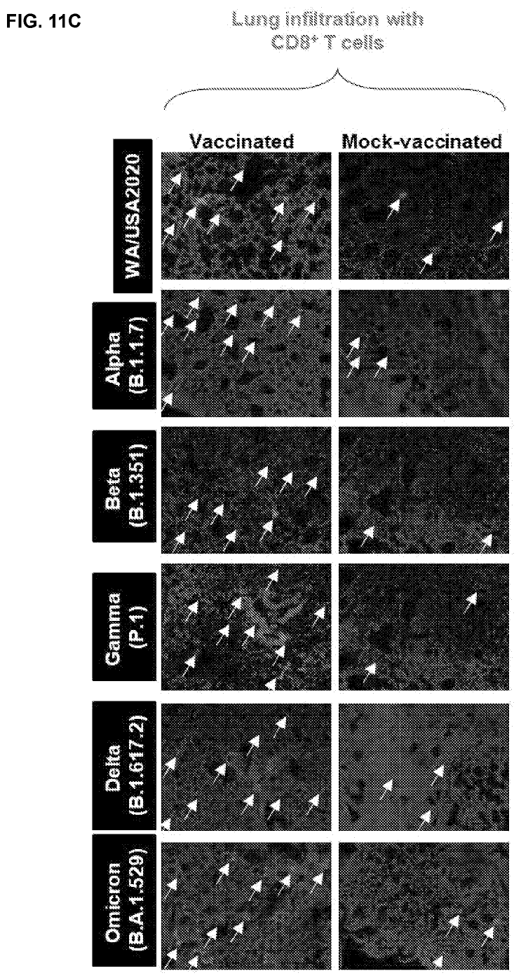

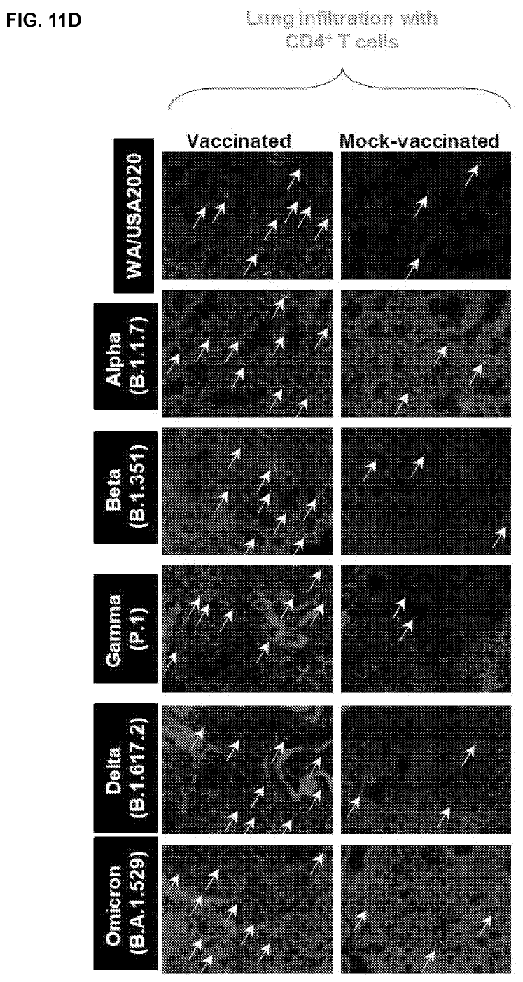

Immunization with the Pan-Coronavirus vaccine bearing conserved epitopes reduced COVID-19-related lung pathology and virus replication associated with increased infiltration of CD8⁺ and CD4⁺ T cells in the lungs: Hematoxylin and eosin staining of lung sections at day 14 p.i. showed a significant reduction in COVID-19-related lung pathology in the mice immunized with conserved Pan-Coronavirus vaccine compared to mock-vaccinated mice (FIG. 11A). This reduction in lung pathology was observed for all six SARS-CoV-2 variants: USA-WA1/2020, Alpha (B.1.1.7), Beta (B.1.351), Gamma (P.1), Delta (B.1.617.2), and Omicron (B.1.1.529) showed severe lung pathogenicity (FIG. 11A). SARS-CoV-2 Nucleocapsid Antibody-based Immunohistochemistry (IHC) staining was further performed on lung tissues obtained from vaccinated and mock-vaccinated groups of mice infected with SARS-CoV-2 variants. Significantly lower antibody staining was detected in the lung tissues of the vaccinated compared mock-vaccinated group of mice following infection with each of the six SARS-CoV-2 variants of concern. This indicated higher expression of the target viral proteins in the lungs of the mock-vaccinated compared to the vaccinated group of mice (FIG. 11B). Furthermore, IHC staining was performed to compare the infiltration CD8⁺ and CD4⁺ T cells into lung tissues of vaccinated and mock-vaccinated mice infected with various SARS-CoV-2 variants. Fourteen days following infection with each of the six variants, we observed a significant increase in the infiltration of both CD8⁺ T cells (FIG. 11C) and CD4⁺ T cells (FIG. 11D) in the lungs of vaccinated mice compared to mock-vaccinated mice.

Altogether these results indicate that immunization with the Pan-Coronavirus vaccine bearing conserved epitopes induced cross-protective CD8⁺ and CD4⁺ T cells that infiltrated the lungs cleared the virus, and reduced COVID-19-related lung pathology following infection with various multiple SARS-CoV-2 variants.

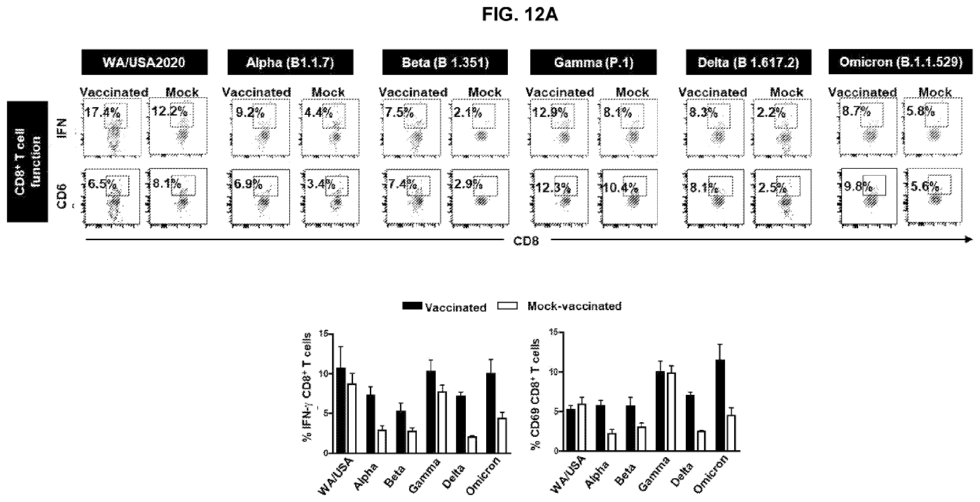

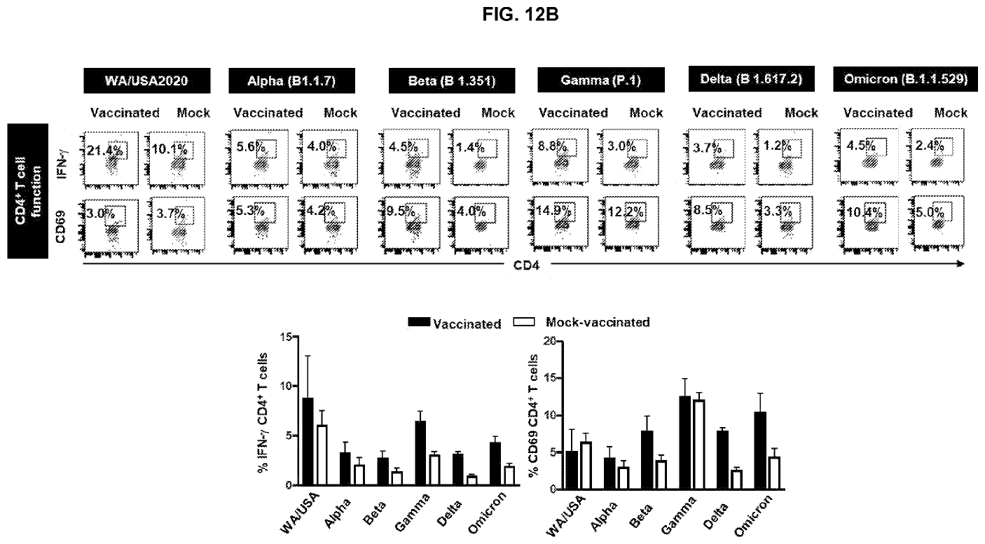

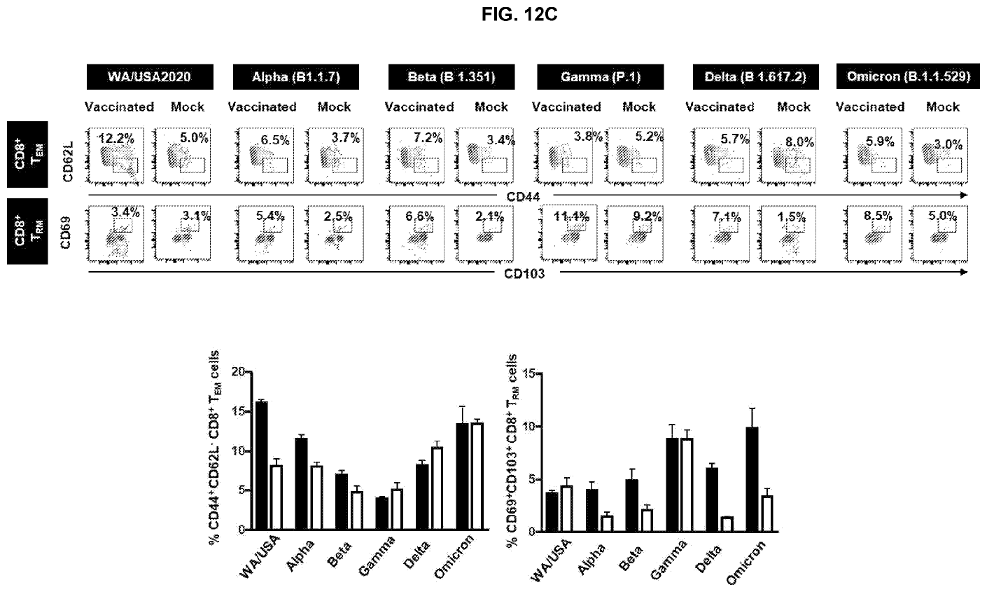

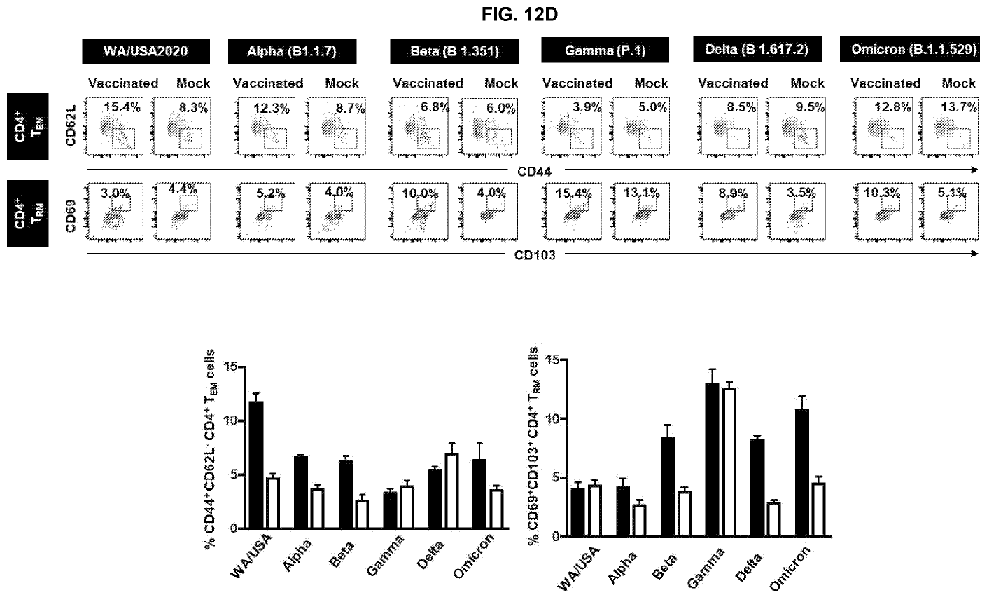

Increased frequencies of lung-resident functional CD8⁺ and CD4⁺ T_{EM} and T_{RM} cells induced by the Pan-Coronavirus vaccine are associated with protection against multiple SARS-CoV-2 variants: To determine whether increased frequencies of lung-resident functional CD8⁺ and CD4⁺ T cells induced by the pan-Coronavirus vaccine are associated with protection against multiple SARS-CoV-2 variants, flow cytometry was used and the frequencies of IFN-γ CD8⁺ T cells and CD69 CD8⁺ T cells were compared (FIG. 12A), IFN-γ CD4⁺ T cells and CD69 CD4⁺ T cells (FIG. 12B) in cell suspensions from the lungs of vaccinated versus mock-vaccinated groups of mice.

Relatively higher frequencies of IFN-γ CD8⁺ T cells were detected in the lungs of protected mice that received the pan-Coronavirus vaccine compared to non-protected mock-vaccinated mice following infections with various SARS-CoV-2 variants: USA-WA 1/2020 (Vaccinated=17.4% vs. Mock=12.2%, P=0.5178), Alpha (B.1.1.7) (Vaccinated=9.2% vs. Mock=4.4%, P=0.0076), Beta (B.1.351) (Vaccinated=7.5% vs Mock=2.1%, P=0.05), Gamma (P.1) (Vaccinated=12.9% vs. Mock=8.1%, P=0.14), Delta (B.1.617.2) (Vaccinated=8.3% vs. Mock=2.23%, P<0.0001), and Omicron (B.1.1.529) (Vaccinated=8.7% vs. Mock=5.8%, P=0.02) (FIG. 12A, top row). Similarly, increased frequencies for CD8+CD69+ T cells were detected in the lungs of protected mice that received the pan-Coronavirus vaccine compared to non-protected mock-vaccinated mice following infections with various SARS-CoV-2 variants: Alpha (B.1.1.7) (Vaccinated=6.9% vs Mock=3.4%, P=0.0033), Beta (B.1.351) (Vaccinated=7.4% vs Mock=2.9%, P=0.05), Gamma (P.1) (Vaccinated=12.3% vs Mock=10.4%, P=0.95), Delta (B.1.617.2) (Vaccinated=8.1% vs Mock=2.5%, P<0.0001), and Omicron (B.1.1.529) (Vaccinated=9.8% vs Mock=5.6%, P=0.01) (FIG. 12A, bottom row).

Moreover, higher frequencies of IFN-γ CD4+ T cells were detected in the lungs of protected mice that received the pan-Coronavirus vaccine compared to non-protected mock-vaccinated mice following infections with various SARS-CoV-2 variants: USA-WA1/2020 (Vaccinated=21.4% vs Mock=10.1%, P=0.5696), Alpha (B.1.1.7) (Vaccinated=5.6% vs Mock=4%, P=0.35), Beta (B.1.351) (Vaccinated=4.5% vs Mock=1.4% %, P=0.12), Gamma (P.1) (Vaccinated=8.8% vs Mock=3%, P=0.02), Delta (B.1.617.2) (Vaccinated=3.7% vs Mock=1.2%, P=0.0002), and Omicron (B.1.1.529) (Vaccinated=4.5% vs Mock=2.4%, P=0.01) (FIG. 12B, top row). Similarly, increased frequencies for CD4+CD69+ T cells were detected in the lungs of protected mice that received the pan-Coronavirus vaccine compared to non-protected mock-vaccinated mice following infections with various SARS-CoV-2 variants: Alpha (B.1.1.7) (Vaccinated=5.3% vs Mock=4.2%, P=0.1748), Beta (B.1.351) (Vaccinated=9.5% vs Mock=4%, P=0.009), Gamma (P.1) (Vaccinated=14.9% vs Mock=12.2%, P=0.7155), Delta (B.1.617.2) (Vaccinated=8.5% vs Mock=3.3%, P<0.0001), and Omicron (B.1.1.529) (Vaccinated=10.4% vs Mock=5%, P=0.003) (FIG. 12B, bottom row).

FACS-based immunophenotyping, confirmed higher frequencies of the memory CD8+T_{EM} (CD44+CD62L) cell subset in immunized mice with a pool of pan-Coronavirus peptides and subjected to infection against USA-WA1/2020 (Vaccinated=12.2% vs Mock=5%, P<0.0001), Alpha (B.1.1.7) (Vaccinated=6.5% vs Mock=3.7%, P=0.0017), Beta (B.1.351) (Vaccinated=7.2% vs Mock=3.4%, P=0.0253), and Omicron (B.1.1.529) (Vaccinated=5.9% vs Mock=3%, P=0.9765) (FIG. 12C). Similarly, when the frequencies for the memory CD8⁺ T_{RM} (CD69⁺CD103⁺) cell subset was evaluated, higher CD8⁺ T_{RM} cell subset frequencies was found for immunized mice infected with USA-WA1/2020 (Vaccinated=3.4% vs Mock=3.1%, P=0.4004), Alpha (B.1.1.7) (Vaccinated=5.4% vs Mock=2.5%, P=0.0160), Beta (B.1.351) (Vaccinated=6.6% vs Mock=2.1%, P=0.0420), Gamma (P.1) (Vaccinated=11.1% vs Mock=9.2%, P=0.9961), Delta (B.1.617.2) (Vaccinated=7.1% vs Mock=1.5%, P<0.0001), and Omicron (B.1.1.529) (Vaccinated=8.5% vs Mock=5%, P=0.0139) (FIG. 12C).

Moreover, in context to memory CD4⁺ T_{EM} (CD44⁺CD62L) cell subset, relatively higher frequencies were observed for immunized mice subjected to infection with SARS-CoV-2 variants USA-WA1/2020 (Vaccinated=15.4% vs Mock=8.3%, P=0.0001), Alpha (B.1.1.7) (Vaccinated=12.3% vs Mock=8.7%, P<0.0001), and Beta (B.1.351) (Vaccinated=6.8% vs Mock=6%, P<0.0004) (FIG. 12D). Higher frequencies of the CD4+T_{RM} (CD69⁺CD103⁺) cell subset were found in immunized mice infected with SARS-CoV-2 variants Alpha (B.1.1.7) (Vaccinated=5.2% vs Mock=4%, P=0.0828), Beta (B.1.351) (Vaccinated=10% vs Mock=4%, P=0.005), Gamma (P.1) (Vaccinated=15.4% vs Mock=13.1%, P=0.7860), Delta (B.1.617.2) (Vaccinated=8.9% vs Mock=3.5%, P<0.0001), and Omicron (B.1.1.529) (Vaccinated=10.3% vs Mock=5.1%, P=0.0021) (FIG. 12D).

Altogether, aforementioned findings confirm that immunization with the pan-Coronavirus vaccine bearing conserved epitopes induced high frequencies of functional CD8⁺ and CD4⁺ T_{EM} and T_{RM} cells that infiltrate the lungs associated with a significant decrease in virus replication and a reduction in COVID-19-related lung pathology following infection with various multiple SARS-CoV-2 variants.

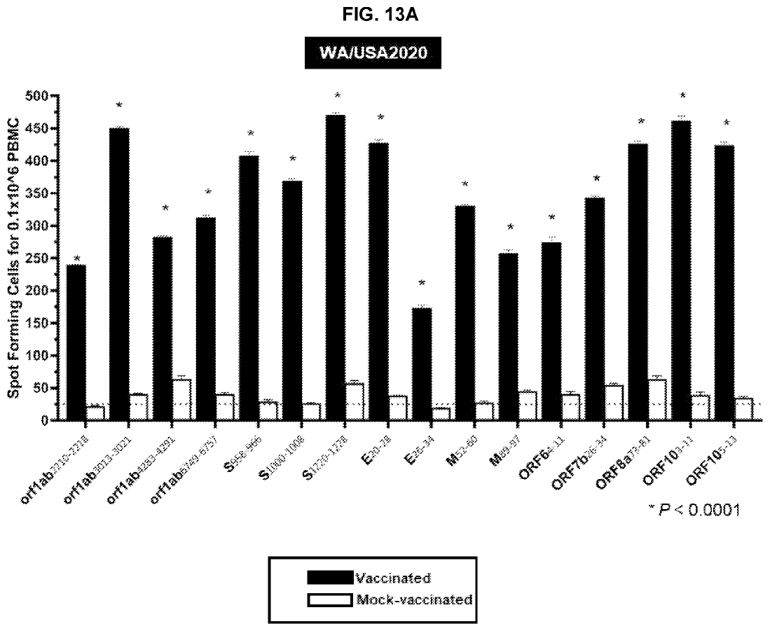

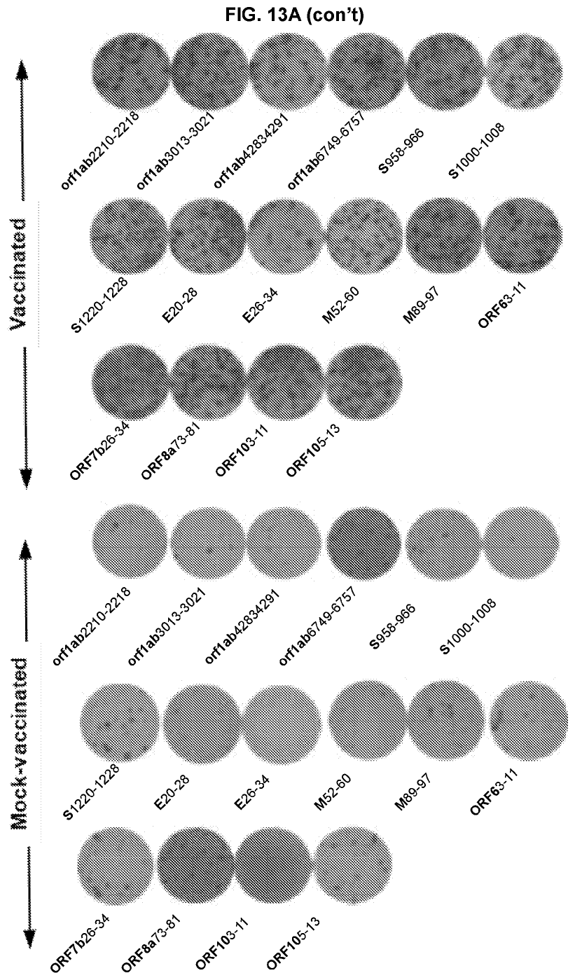

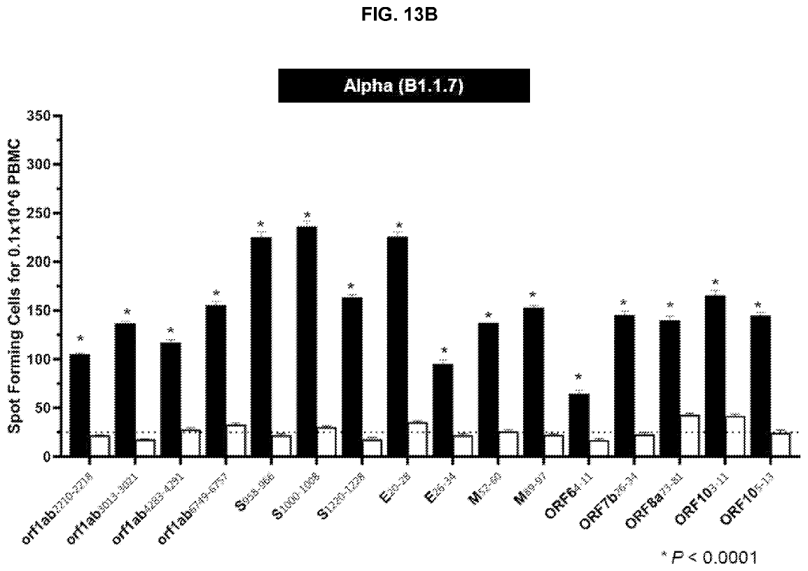

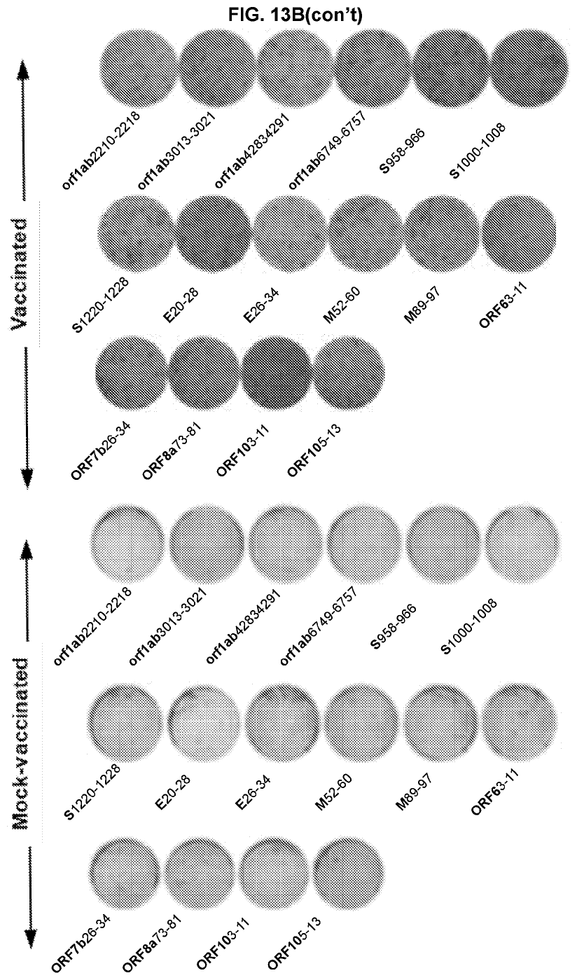

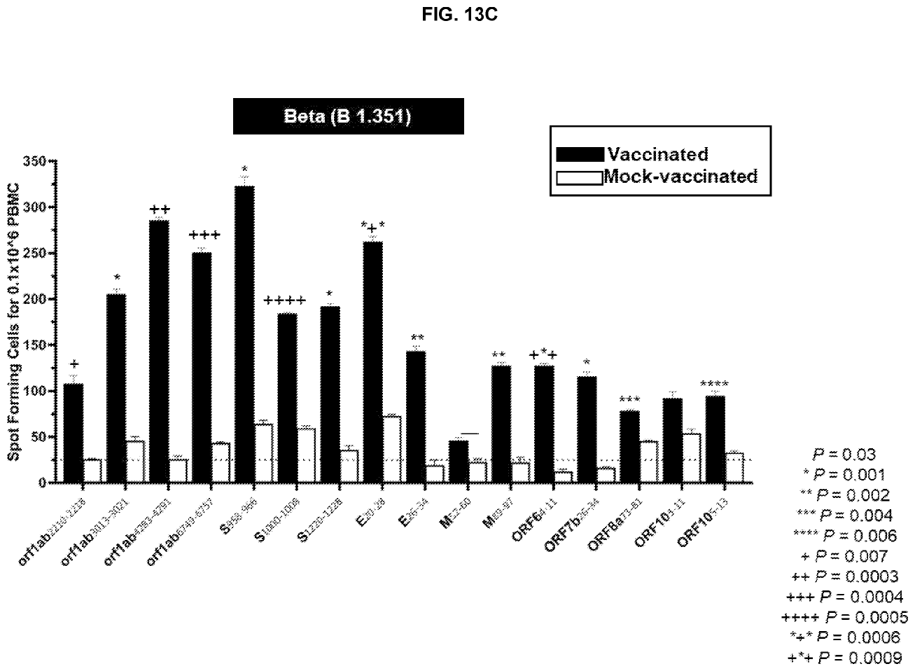

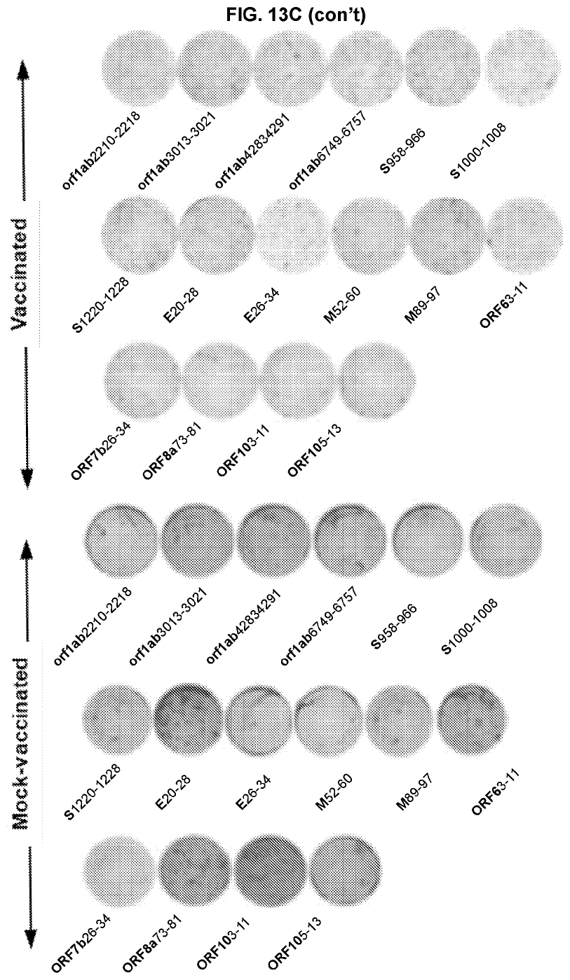

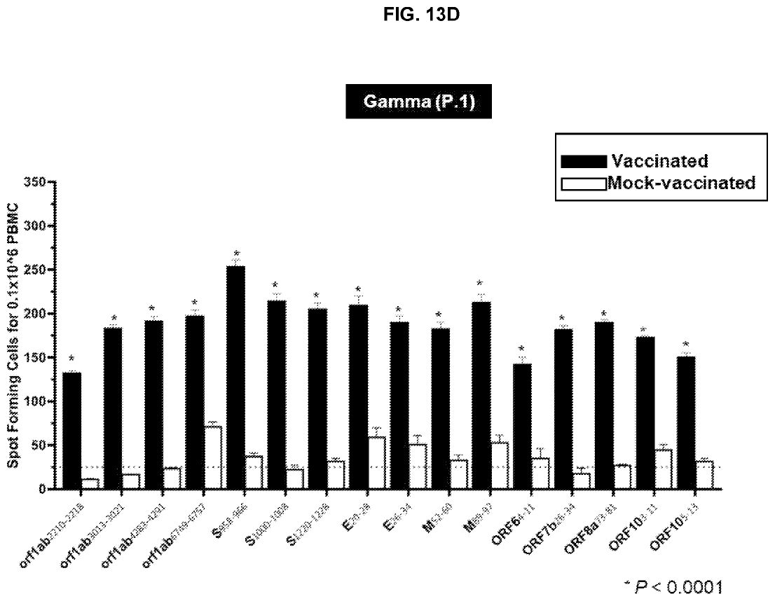

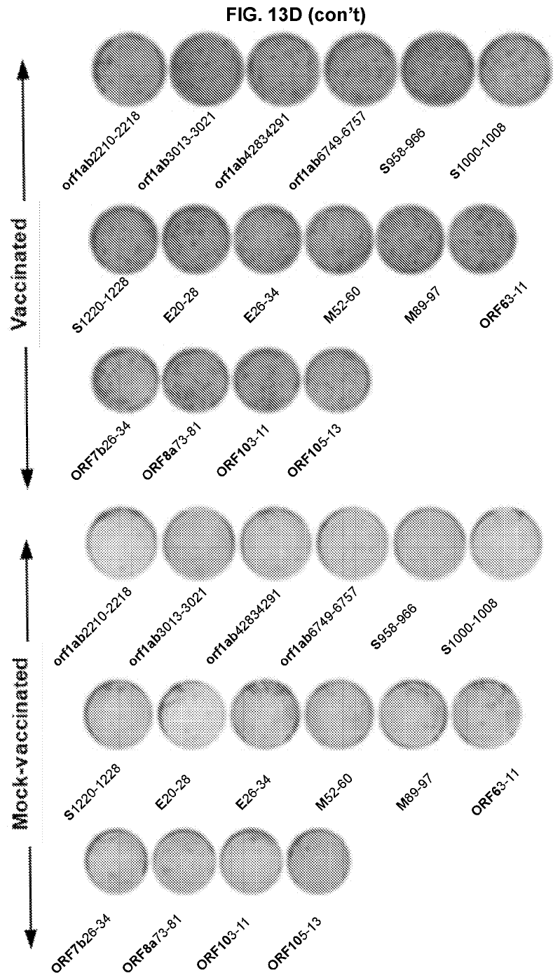

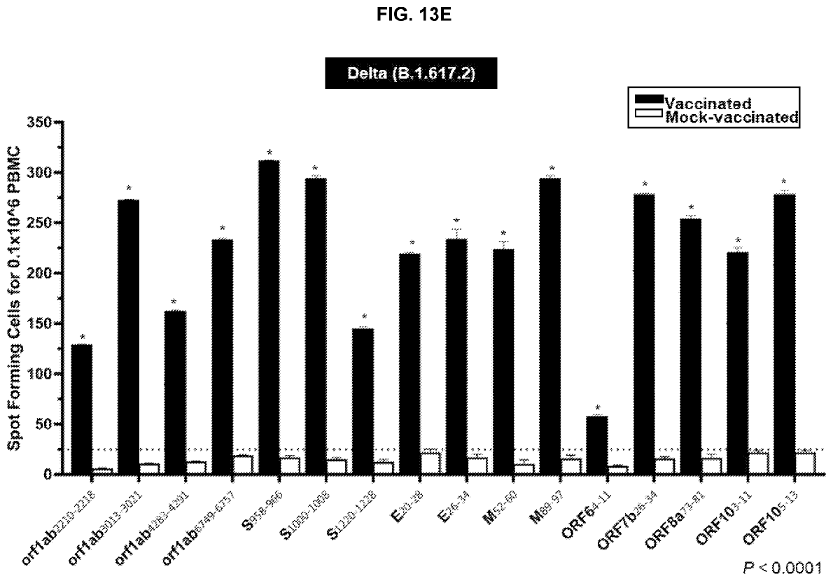

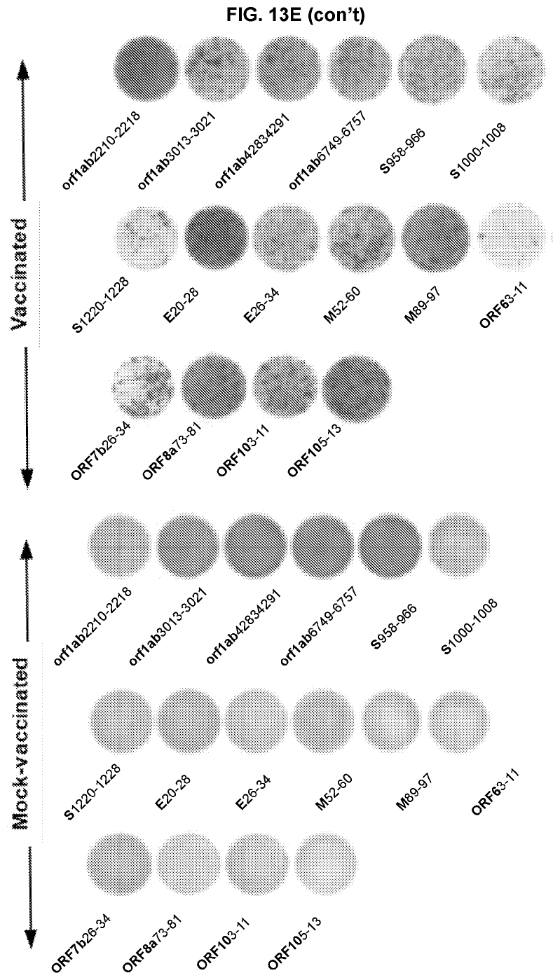

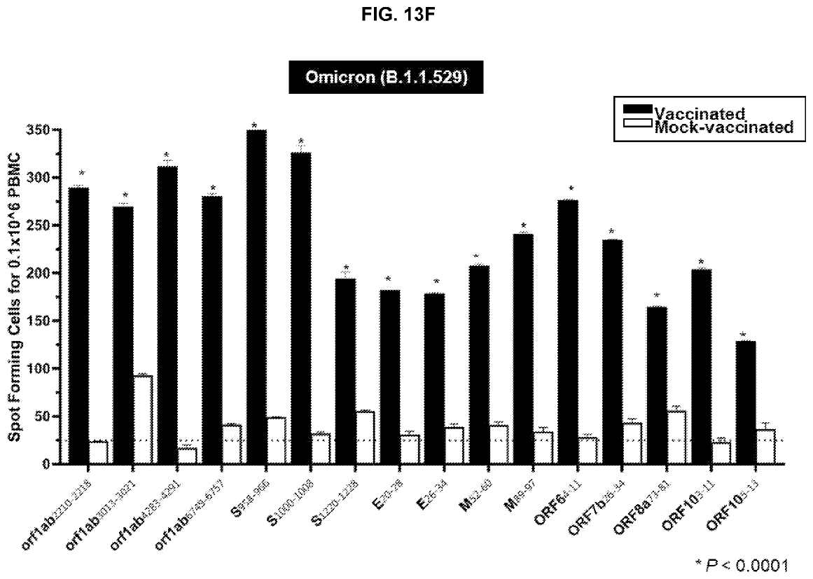

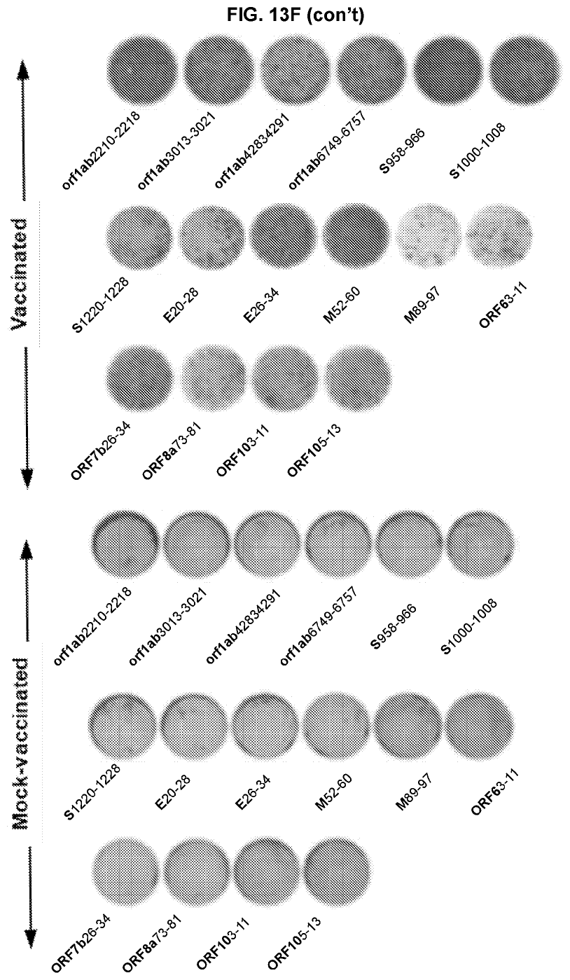

Increased SARS-CoV-2 epitopes-specific IFN-γ-producing CD8⁺ T cells in the lungs of vaccinated mice in comparison to mock-vaccinated mice: To determine whether the functional lung-resident CD8⁺ T cells are specific to SARS-CoV-2, lung-cell suspension from vaccinated and mock-vaccinated mice were stimulated with each of the 14 “universal” human CD8+ T cell epitopes (ORF1 ab₂₂₁₀₋₂₂₁₈, ORF1ab₃₀₁₃₋₃₀₂₁, ORF1ab₄₂₈₃₋₄₂₉₁, ORF1ab₆₇₄₉₋₆₇₅₇; ORF6₃₋₁₁, ORF7b₂₆₋₃₄, ORF10₃₋₁₁, ORF10₅₋₁₃, S₉₅₈₋₉₆₆, S₁₀₀₀₋₁₀₀₈, S₁₂₂₀₋₁₂₂₈, E₂₀₋₂₈, M₅₂₋₆₀, and M₈₉₋₉₇) and quantified the number of IFN-γ-producing CD8⁺ T cells using ELISpot (FIG. 13A-13E). To determine where cross-reactive IFN-γ-producing CD8⁺ T cell responses will be detected regardless of SARS-CoC-2 variant, the number IFN-γ-producing CD8⁺ T cells were determined in the lung tissues of vaccinated and mock-vaccinated mice after challenge with each of six different SARS-CoV-2 variants of concern.

Overall, a significant increase in the number of IFN-γ-producing CD8⁺ T cells was detected in the lungs of protected mice that received the pan-Coronavirus vaccine compared to non-protected mock-vaccinated mice (mean SFCs>25 per 0.5×10⁶ pulmonary immune cells), irrespective of the SARS-CoV-2 variants of concern: WA/USA2020 (FIG. 13A), Alpha (B.1.1.7) (FIG. 13B), Beta (B.1.351) (FIG. 13C), Gamma (P.1) (FIG. 13D), Delta (B.1.617.2) (FIG. 13E), or Omicron (B.1.1.529) (FIG. 13F). All the comparisons among vaccinated and mock-vaccinated groups of mice, irrespective of SARS-CoV-2 variants of concern were found to be statistically significant regardless of whether CD8⁺ T cells targeted epitopes were from structural (Spike, Envelope, Membrane), or non-structural (ORF1ab, ORF6, ORF7b, ORF10) SARS-CoV-2 protein antigens (P<0.5).

Taken together, these results: (1) Confirm that immunization with the pan-Coronavirus vaccine bearing conserved epitopes induced high frequencies of functional CD8⁺ T cells that infiltrate the lungs associated with cross-protection against multiple SARS-CoV-2 variants: (2) Demonstrate that increased SARS-CoV-2 epitopes-specific IFN-γ-producing CD8⁺ T cells in the lungs of vaccinated triple transgenic HLA-A*02:01/HLA-DRB1*01:01-hACE-2 mice are associated with protection from multiple variants of concern. In contrast, low frequencies of lung-resident SARS-CoV-2-specific IFN-γ-producing CD8⁺ T cells were associated with severe disease onset in mock-vaccinated triple transgenic HLA-A*02:01/HLA-DRB1*01:01-hACE-2 mice. In this report, we suggest an important role for functional lung-resident SARS-CoV-2-specific CD8+ T cells specific to highly conserved “universal” epitopes from structural and non-structural antigens in cross-protection against SARS-CoV-2 VOCs.

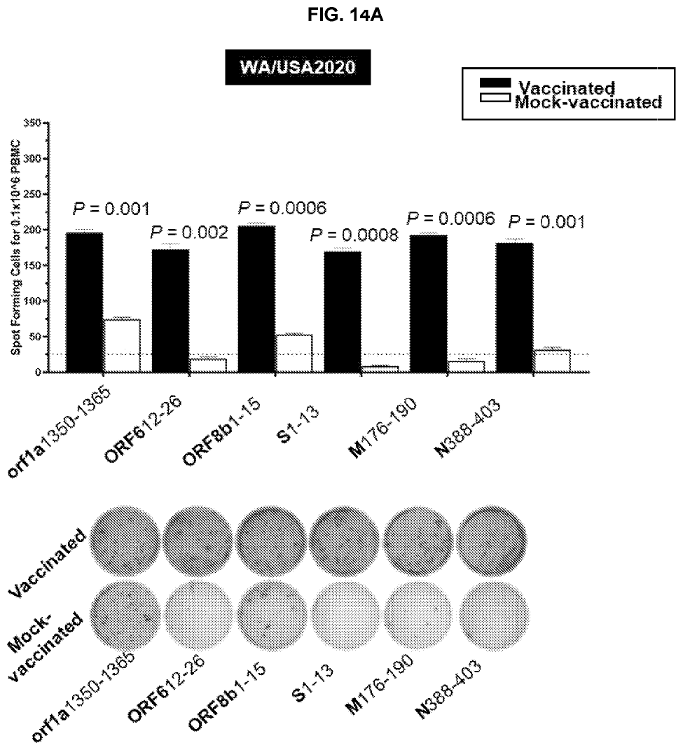

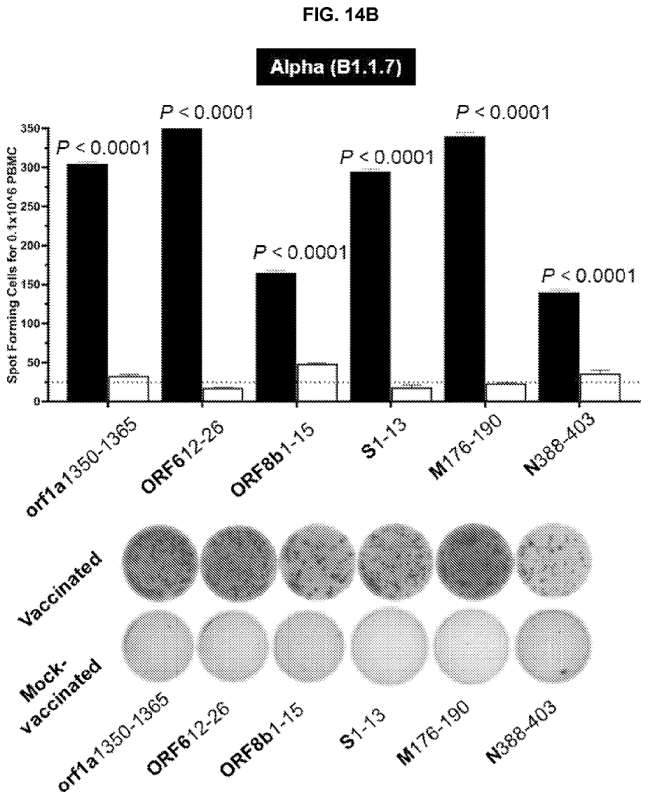

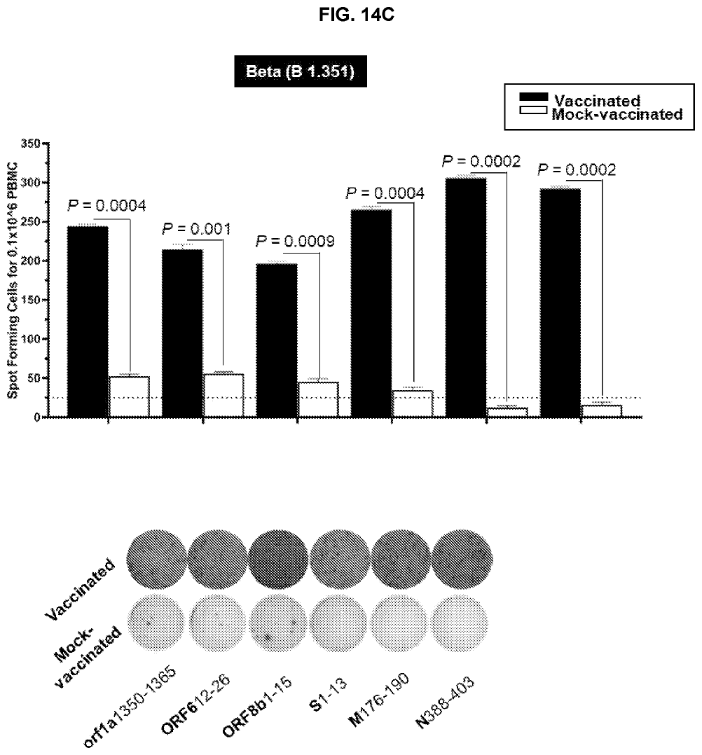

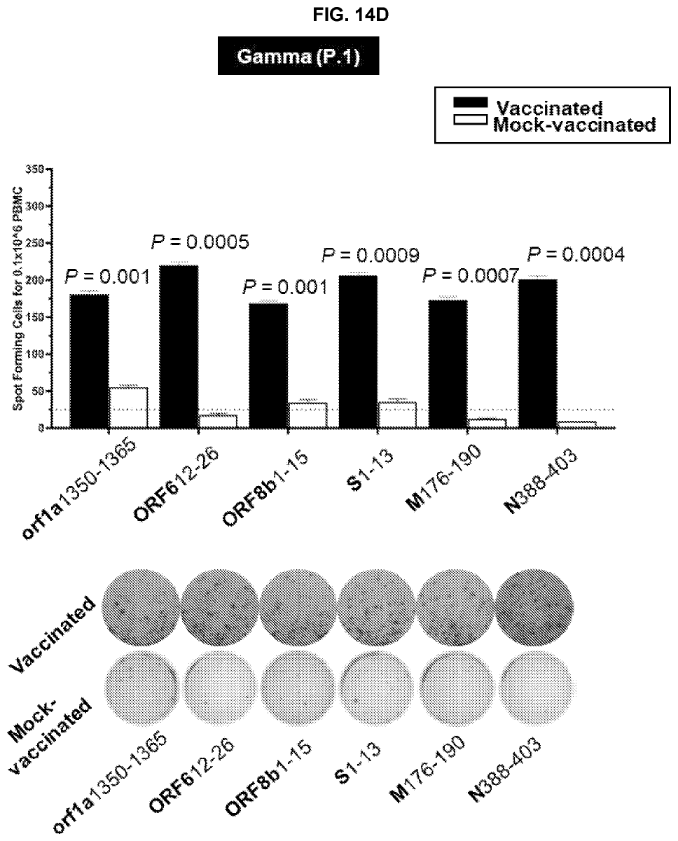

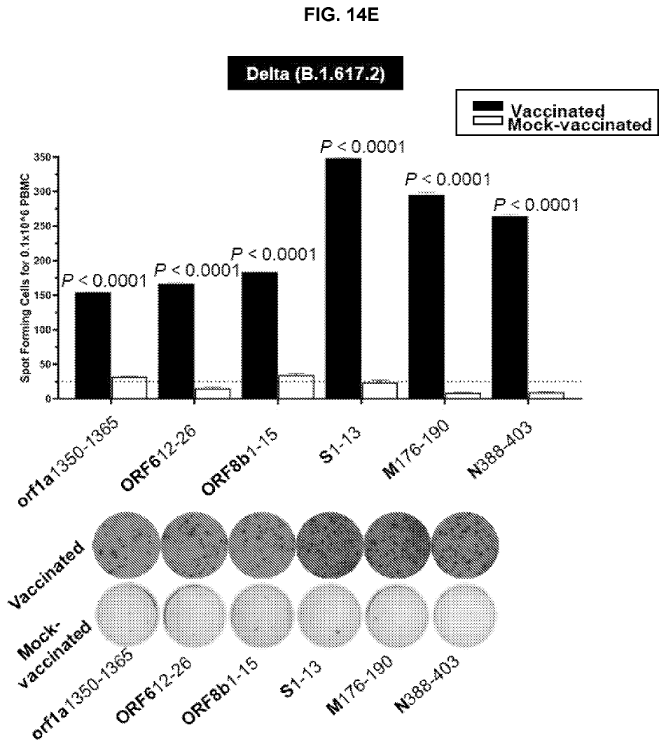

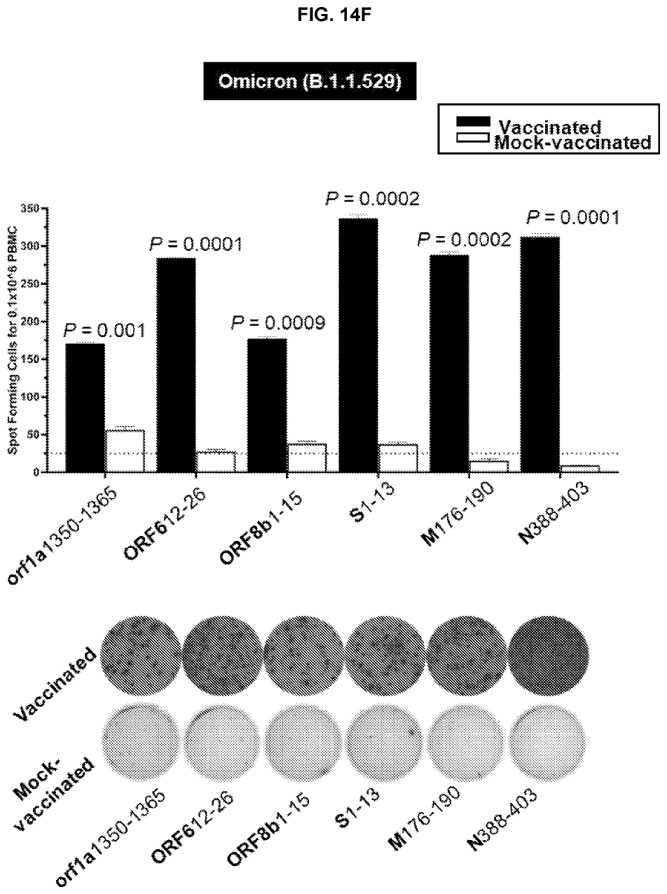

Increased SARS-CoV-2 epitopes-specific IFN-γ-producing CD4⁺ T cells in the lungs of vaccinated mice in comparison to mock-vaccinated mice: Lung-cell suspension from vaccinated and mock-vaccinated groups of mice were stimulated with each of the 6 “universal” human CD4⁺ T cell epitopes (ORF1a₁₃₅₀₋₁₃₆₅, ORF6₁₂₋₂₆, ORF8b₁₋₁₅, S₁₋₁₃, M₁₇₆₋₁₉₀, and N₃₈₈₋₄₀₃) and quantified the number of IFN-γ-producing CD4⁺ T cells using ELISpot, to determine whether the functional lung-resident CD4⁺ T cells are specific to SARS-CoV-2 (FIG. 14A-14E).

Overall, a significant increase in the number of IFN-γ-producing CD4⁺ T cells were detected in the lungs of protected mice that received the pan-Coronavirus vaccine compared to non-protected mock-vaccinated mice (mean SFCs>25 per 0.5×10⁶ pulmonary immune cells), irrespective of the SARS-CoV-2 VOCs: WA/USA2020 (FIG. 14A), Alpha (B.1.1.7) (FIG. 14B), Beta (B.1.351) (FIG. 14C), Gamma (P.1) (FIG. 14D), Delta (B.1.617.2) (FIG. 14E), or Omicron (B.1.1.529) (FIG. 14F). All the comparisons among vaccinated and mock-vaccinated groups of mice, irrespective of SARS-CoV-2 VOCs were statistically significant regardless of whether CD4+ T cells targeted epitopes were from structural or non-structural SARS-CoV-2 protein antigens (P<0.5).

Taken together, the aforementioned findings demonstrate that increased SARS-CoV-2 epitopes-specific IFN-γ-producing CD4⁺ T cells in the lungs of vaccinated triple transgenic HLA-A*02:01/HLA-DRB1*01:01-hACE-2 mice are associated with protection from multiple variants of concern. In contrast, low frequencies of lung-resident SARS-CoV-2-specific IFN-γ-producing CD4+ T cells were associated onset with severe disease in mock-vaccinated triple transgenic HLA-A*02:01/HLA-DRB1*01:01-hACE-2 mice. The findings suggest an important role of functional lung-resident SARS-CoV-2-specific CD4+ T cells specific to highly conserved “universal” epitopes from structural and non-structural antigens in cross-protection against SARS-CoV-2 VOCs.

Universal B cell epitopes from SARS-CoV-2 Spike protein showed a high degree of immunogenicity across SARS-CoV-2 variants based on antibody response in COVID-19 patients and triple transgenic HLA-A*02:01/HLA-DRB1*01:01-hACE-2: Next, whether the antibody responses were associated with protection was determined, since the prototype pan-Coronavirus vaccine used herein also contains nine conserved B cell epitopes selected from the Spike glycoprotein of SARS-CoV-2. The nine B-cell epitopes were screened for their conservancy against variants namely h-CoV-2/Wuhan (MN908947.3), h-CoV-2/WA/USA2020 (OQ294668.1), h-CoV-2/Alpha (B1.1.7) (OL689430.1), h-CoV-2/Beta (B 1.351) (MZ314998), h-CoV-2/Gamma (P.1) (MZ427312.1), h-CoV-2/Delta (B.1.617.2) (OK091006.1), and h-CoV-2/Omicron (B.1.1.529) (OM570283.1). 100% conservancy was observed in three of the earlier predicted B cell epitopes namely S₂₈₇₋₃₁₇, S₅₂₄₋₅₅₈, and S₅₆₅₋₅₉₈.

The antibody titer specific to each of the nine “universal” B-cell epitopes was determined by ELISA in COVID-19 patients infected with multiple SARS-CoV-2 variants of concern and in vaccinated and mock-vaccinated triple transgenic HLA-A*02:01/HLA-DRB1*01:01-hACE-2 mice challenged with same SARS-CoV-2 VOCs. The peptide binding IgG level was significantly higher for all nine “universal” B cell epitopes in COVID-19 patients as well as in vaccinated triple transgenic mice, irrespective of SARS-CoV-2 variant. Reduced peptide binding IgG level was observed for severely ill COVID-19 patients and in mock-vaccinated triple transgenic HLA-A*02:01/HLA-DRB1*01:01-hACE-2 mice.

Altogether, the aforementioned results indicate that immunization with the pan-Coronavirus vaccine bearing conserved “universal” B and T cell epitope induced cross-protective antibodies, CD8+ and CD4+ T cells that infiltrated the lungs, cleared the virus, and reduced COVID-19-related lung pathology following infection with various multiple SARS-CoV-2 VOCs.

Viruses: SARS-CoV-2 viruses specific to six variants, namely (i) SARS-CoV-2-USA/WA/2020 (Batch Number: G2027B); (ii) Alpha (B.1.1.7) (isolate England/204820464/2020 Batch Number: C2108K); (iii) Beta (B.1.351) (isolate South Africa/KRISP-EC-K005321/2020; Batch Number: C2108F), (iv) Gamma (P.1) (isolate hCoV-19/Japan/TY7-503/2021; Batch Number: G2126A), (v) Delta (B.1.617.2) (isolate h-CoV-19/USA/MA29189; Batch number: G87167), and Omicron (BA.1.529) (isolate h-CoV-19/USA/FL17829; Batch number: G76172) were procured from Microbiologics (St. Cloud, MN). The initial batches of viral stocks were propagated to generate high-titer virus stocks. Vero E6 (ATCC-CRL1586) cells were used. Procedures were completed only after appropriate safety training was obtained using an aseptic technique under BSL-3 containment.

Triple transgenic mice immunization with SARS-CoV-2 conserved peptides and Infection: The University of California-Irvine conformed to the Guide for the Care and Use of Laboratory Animals published by the US National Institute of Health (IACUC protocol #AUP-22-086). Seven to eight-week-old triple transgenic HLA-A*02:01/HLA-DRB1*01:01-hACE-2 mice (n=60) were included in this experiment. Mice were subcutaneously immunized with a pool of conserved Pan-Coronavirus peptides. The peptide pool administered per mouse comprised 25 μg each of the 9-mer long 16 CD8+ T cell peptides (ORF1ab₂₂₁₀₋₂₂₁₈, ORF1ab₃₀₁₃₋₃₀₂₁, ORF1ab₄₂₈₃₋₄₂₉₁, ORF1ab₆₇₄₉₋₆₇₅₇, ORF6₃₋₁₁, ORF7b₂₆₋₃₄, ORF8a₇₃₋₈₁, ORF10₃₋₁₁, ORF10₅₋₁₃, S₉₅₈₋₉₆₆, S₁₀₀₀₋₁₀₀₈, S₁₂₂₀₋₁₂₂₈, E₂₀₋₂₈, E₂₆₋₃₄, M₅₂₋₆₀, and M₈₉₋₉₇), 15-mer long 6 CD4⁺ T cell epitopes (ORF1a₁₃₅₀₋₁₃₆₅, ORF6₁₂₋₂₆, ORF8b₁₋₁₅, S₁₋₁₃, M₁₇₆₋₁₉₀, and N₃₈₈₋₄₀₃), and 9 B-cell peptides. The pool of peptides was then mixed with 25 μg of CpG and 25 μg of Alum to prepare the final composition. Mice were immunized with the peptide pool on Day 0 and Day 14 of the experiment. Fourteen days following the second immunization, on Day 28, mice were divided into 6 groups and intranasally infected with 1×10⁵ pfu of SARS-CoV-2 (USA-WA1/2020) (n=10), 6×10³ pfu of SARS-CoV-2-Alpha (B.1.1.7) (n=10), 6×10³ pfu of SARS-CoV-2-Beta (B.1.351) (n=10), 5×10² pfu of SARS-CoV-2-Gamma (P.1) (n=10), 8×10³ pfu of SARS-CoV-2-Delta (B.1.617.2) (n=10), and 6.9×10⁴ pfu of SARS-CoV-2-Omicron (B.1.1.529) (n=10). The viruses were diluted, and each mouse was administered intranasally with 20 μl volume. Mice were monitored daily for weight loss and survival until Day 14 p.i. Throat swabs were collected for viral titration on Days 2, 4, 6, 8, 10, and 14 post-infection.

Human study population cohort and HLA genotyping: In this study, we have included 210 subjects from a pool of over 682 subjects. Written informed consent was obtained from participants before inclusion. The subjects were categorized as mild to severe COVID-19 groups and have undergone treatment at the University of California Irvine Medical Center between July 2020 to July 2022 (Institutional Review Board protocol #-2020-5779). SARS-CoV-2 positivity was defined by a positive RT-PCR on nasopharyngeal swab samples. All the subjects were genotyped by PCR for class I HLA-A*02:01 and class II HLA-DRB1*01:01 among the 682 patients (and after excluding a few for which the given amount of blood was insufficient—i.e., less than 6 ml), we ended up with 210 that were genotyped for HLA-A*02:01″ or/and HLA-DRB1*01:01+. Based on the severity of symptoms and ICU admission/intubation status, the subjects were divided into five broad severity categories namely: Severity 5: patients who died from COVID-19 complications; Severity 4: infected COVID-19 patients with severe disease that were admitted to the intensive care unit (ICU) and required ventilation support; Severity 3: infected COVID-19 patients with severe disease that required enrollment in ICU, but without ventilation support; Severity 2: infected COVID-19 patients with moderate symptoms that involved a regular hospital admission; Severity 1: infected COVID-19 patients with mild symptoms; and Severity 0: infected individuals with no symptoms. Demographically, the 210 patients included were from mixed ethnicities (Hispanic (34%), Hispanic Latino (29%), Asian (19%), Caucasian (14%), Afro-American (3%), and Native Hawaiian and Other Pacific Islander descent (1%).

### Example 3: a Multi-Epitope/CXCL11 Prime/Pull Coronavirus Mucosal Vaccine Boosts the Frequency and the Function of Lung-Resident CD4+ and CD8+Memory T Cells and Protects Against COVID-19-Like Symptoms and Death Caused by SARS-CoV-2 Infection

The present invention features a pre-clinically tested the safety, immunogenicity, and protective efficacy of a novel multi-epitope//CXCL11 prime/pull mucosal Coronavirus vaccine. This prime/pull vaccine strategy comprises intranasal delivery of a lung-tropic adeno-associated virus type 9 (AAV-9) vector that incorporates highly conserved human B, CD4+ CD8+ cell epitopes of SARS-CoV-2 (prime) and pulling the primed B and T cells into the lungs using the T cell attracting chemokine, CXCL-11 (pull). The present example demonstrated that immunization of HLA-DR*0101/HLA-A*0201/hACE2 triple transgenic mice with this multi-epitope//CXCL11 prime/pull Coronavirus mucosal vaccine: (i) Increased the frequencies of CD4+ and CD8+ TEM, TCM, and TRM cells in the lungs; and (ii) reduced COVID19-like symptoms, lowered virus replication, and prevented deaths following challenge with SARS-CoV-2. These findings discuss the importance of bolstering the number and function of lung-resident memory CD4+ and CD8+ T cells for better protection against SARS-CoV-2 infection, COVID-19-like symptoms, and death

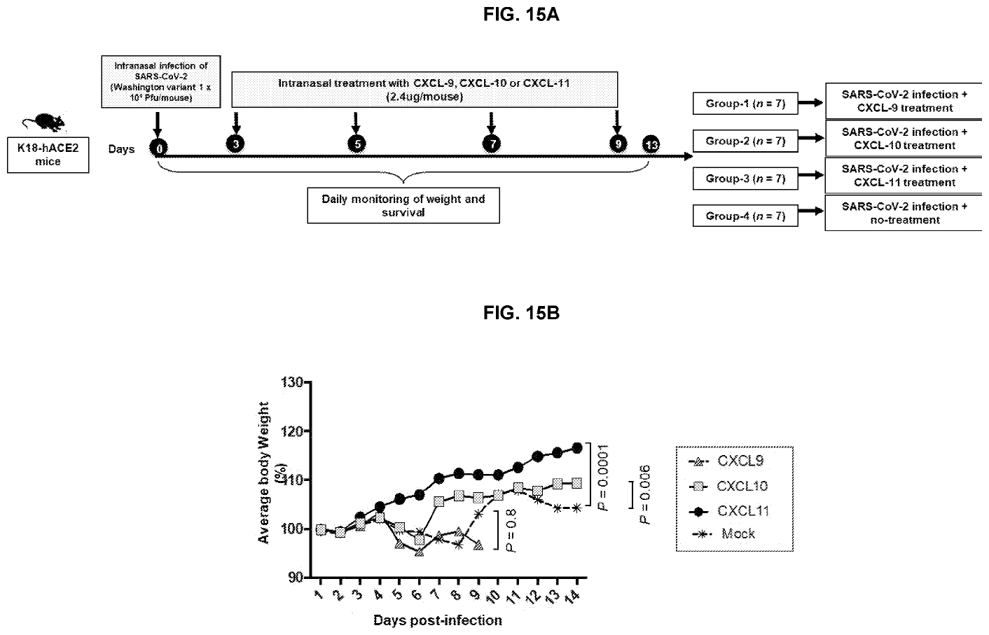

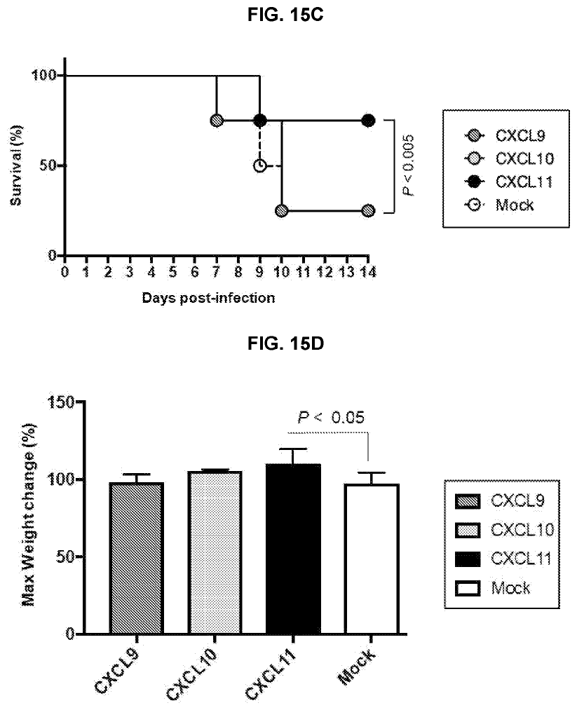

Treatment of SARS-CoV-2-infected K18-hACE2 mice with CXCL-11 T-cell-attracting chemokine improves COVID-19-like symptoms and survivals: We first determined whether treatment of SARS-CoV-2 infected K18-hACE2 mice with CXCL-9, CXCL-10, and CXCL-11 T-cell-attracting chemokines will improve COVID-19-like symptoms. Eight to nine-week-old K18-hACE2 transgenic mice (n=20) were first infected intranasally with SARS-CoV-2 (1×10⁴ pfu/mouse of Washington USA-WA1/2020 variant) and were subsequently left untreated (control) or treated intranasally on days 3, 5, 7 and 9 post-infection either with CXCL-9, CXCL-10, or CXCL-11 chemokine (FIG. 15A). Chemokine-treated and untreated mice were then followed daily for the percentage of body weight and survival (i.e., COVID-19-like symptoms and death scoring). SARS-CoV-2-infected and CXCL-11-treated K18-hACE2 mice presented significant protection against weight loss (FIG. 15B and FIG. 15D) and death (FIG. 15C) compared to the SARS-CoV-2-infected untreated control K18-hACE2 mice (P<0.05). However, SARS-CoV-2-infected and CXCL-9-treated K18-hACE2 mice did not show significant improvement in weight loss (FIG. 15B and FIG. 15D) and death (FIG. 15C) compared to the SARS-CoV-2-infected untreated control K18-hACE2 mice (P>0.05). Similar to the CXCL-11-treated K18-hACE2 mice, the CXCL-10-treated K18-hACE2 mice showed better survival (100% survival vs. 75% survival, respectively, FIG. 15C).

Altogether, these results indicate that among the three CXCL-9, CXCL-10, and CXCL-11 T-cell-attracting chemokines; treatment with the CXCL-11 chemokine significantly improved COVID-19-like symptoms and reduced deaths in SARS-CoV-2 infected K18-hACE2 mice.

A multi-epitope/CXCL11 prime/pull Coronavirus vaccine protects against COVID19-like symptoms in HLA-DR*0101/HLA-A*0201/hACE2 triple transgenic mice following infection with SARS-CoV-2: Since treatment with the CXCL-11, but not CXCL-9 and CXCL-10 chemokines, appeared to improve COVID-19-like symptoms and survivals of K18-hACE2 mice following infection with SARS-CoV-2, next it was determined whether CXCL-11 treatment would also improve the protection induced by a multi-epitope coronavirus vaccine.

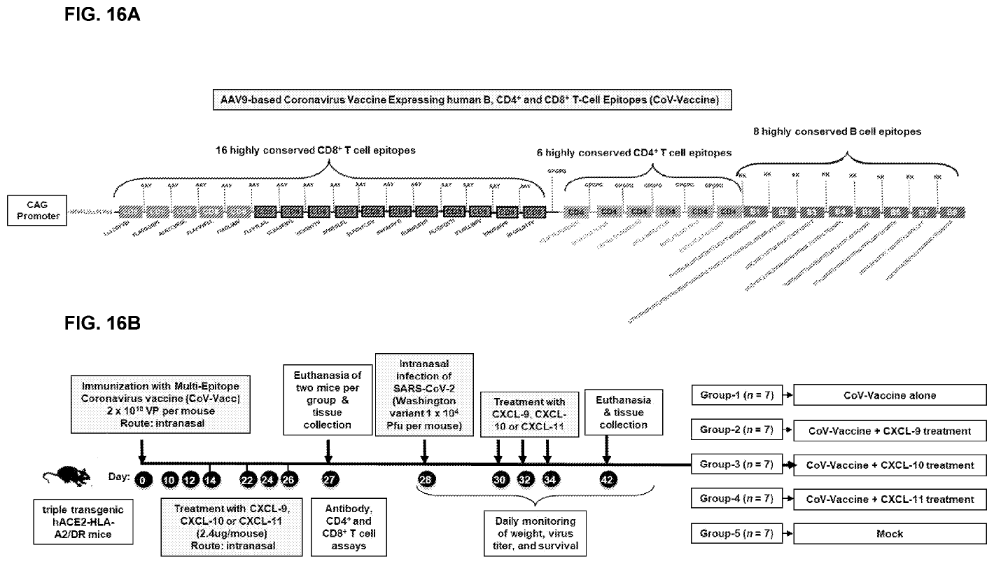

Thus, the protective efficacy of multi-epitope prime/pull Coronavirus vaccine candidates bearing multiple human CD4⁺ and CD8⁺ T cell epitopes were compared. For this experiment: (1) a multi-epitope Coronavirus vaccine was designed and produced that co-express recently identified 16 highly conserved human CD8⁺ T cell epitopes, 6 highly conserved human CD4⁺ T cell epitopes, and 8 highly conserved human B cell epitopes all expressed in tandem under improved CMV promoter (CAG) in a lung-tropic adeno-associated virus type 9 (AAV9) vector (designated as CoV-Vacc, FIG. 16A); and (2) a novel HLA-DR*0101/HLA-A*0201/hACE2 triple transgenic mice expressing human ACE2 was generated, human HLA class 1 (HLA-A*0201) and class 2 (HLA-DR*0101) following challenge with SARS-CoV-2.

As illustrated in FIG. 16B, the prime/pull Coronavirus vaccination consists of (i) first priming of SARS-CoV-2-specific B cells, CD4⁺ T cells, and CD8⁺ T cells in HLA-DR*0101/HLA-A*0201/hACE2 triple transgenic mice using the engineered lung-tropic AAV9 multi-epitope Coronavirus vaccine co-expressing the recently identified immunodominant B, CD4⁺, and CD8⁺ T cell epitopes (i.e., CoV-Vacc) and delivered intranasally followed by (ii) Pulling the “primed” B cells, CD4⁺ T cells, and CD8⁺ T cells into the lungs using mouse CXCL-9, CXCL-10, or CXCL-11 T-cell attracting chemokines delivered intranasally (nose drops).

FIG. 16B shows the HLA-DR*0101/HLA-A*0201/hACE2 triple transgenic mice (eight to nine-week-old, n=35)) were intranasally immunized with 2×10¹⁰ VP per mouse of CoV-Vacc (SEQ ID NO: 36). The immunized mice were divided into 5 groups of 7 mice each and subsequently left untreated (CoV-Vacc. alone) or treated intranasally with 2.4 μg of CXCL-9 (n=7), CXCL-10 (n=7), or CXCL-11 (n=7) on days 10, 12, 14, 22, 24, 26 post-immunization. The fifth control group (n=7) was neither immunized with the CoV-Vacc. nor treated with the chemokines (mock). On day 28 post-immunization, mice were intranasally challenged with 1×10⁴ pfu of SARS-CoV-2 (USA-WA1/2020). Subsequently, all vaccinated groups received additional treatment with CXCL-9, CXCL-10, or CXCL-11 on days 30, 32, and 34 post-immunization. All animals were then monitored for up to day 14 post-infection (p.i.). for weight loss, virus replication in the lungs, and death. On day 14 p.i., mice were euthanized, and lungs were collected for lung inflammation using H & E staining.

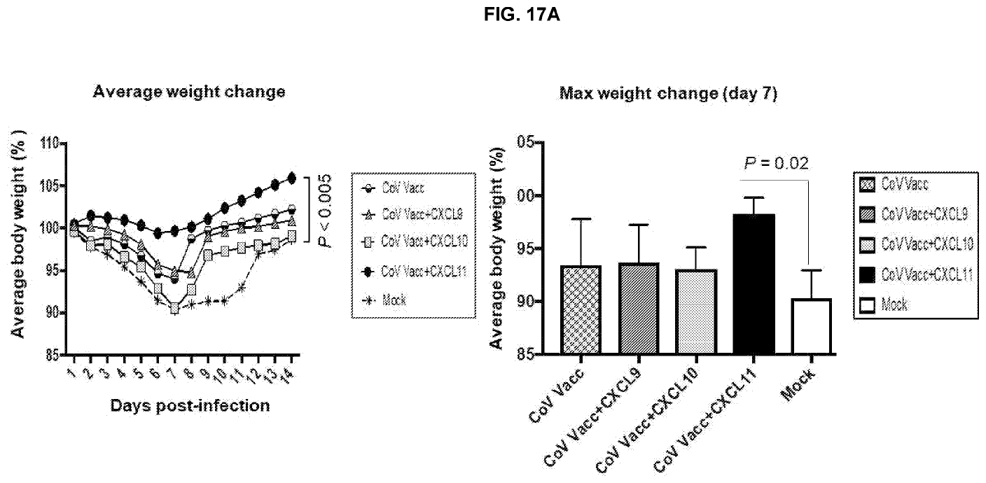

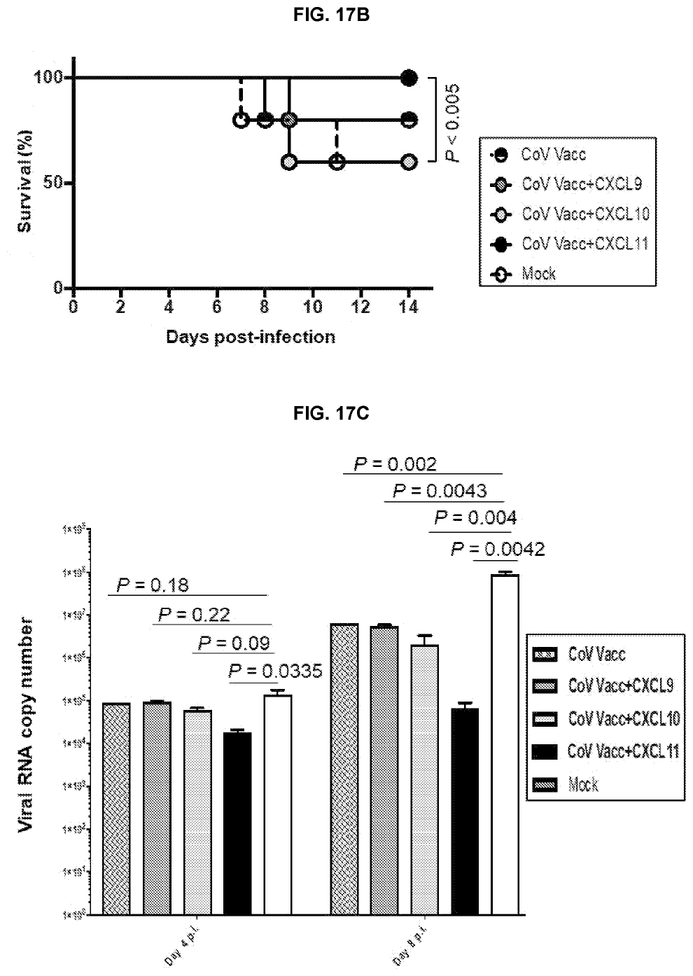

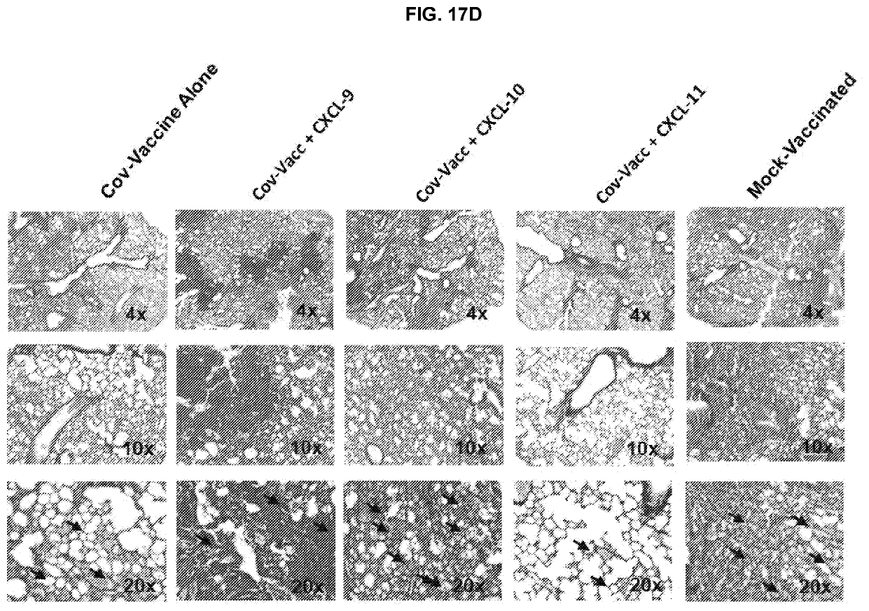

Significant protection against weight loss was observed in the group of mice that received the multi-epitope//CXCL11 prime/pull CoV-Vacc compared to the multi-epitope//CXCL9 prime/pull CoV-Vacc. group, the multi-epitope//CXCL10 prime/pull CoV-Vacc group, as well as the Mock control group (P<0.005, FIG. 17A). Between days 5 and 9 post-challenge with SARS-CoV-2, the multi-epitope//CXCL11 prime/pull CoV-Vacc group lost only around 2% of their initial weight, whereas the remaining groups showed a significant loss in their initial weight that varied between 4% to 10% (FIG. 17A). Accordingly, a significant reduction in virus replication was recorded in the lungs of multi-epitope//CXCL11 prime/pull CoV-Vacc group of mice compared to other groups (FIG. 17C). Significantly lower viral RNA copy numbers were detected on both days 4 and 8 post-infection in the lungs of the multi-epitope//CXCL11 prime/pull CoV-Vacc group compared to other groups (FIG. 17C). Moreover, from day 0 to day 14 post-challenge with SARS-CoV-2, the multi-epitope//CXCL11 prime/pull CoV-Vacc group of mice showed 100% survival whereas the remaining groups depicted only 60% to 80% survival (FIG. 17B). The lowest virus replication in the lungs of multi-epitope//CXCL11 prime/pull CoV-Vacc. group of mice was associated with less inflammatory cells infiltrating the lungs (FIG. 17D). Less pulmonary pathological changes characterized by (i) open alveolar air spaces, (ii) less inflammation, and (iii) less residual cellular debris in air spaces with less alveolar damage, were observed in the H & E sections from mice immunized with Pan-CoV-Vacc and treated with CXCL-11 (FIG. 17D).

Altogether, these results demonstrate that the multi-epitope//CXCL11 prime/pull Coronavirus vaccine protected against COVID19-like symptoms and reduced virus replication and inflammation in the lungs, and prevented deaths in HLA-DR*0101/HLA-A*0201/hACE2 triple transgenic mice following infection with SARS-CoV-2.

Bolstering the frequencies of lung-resident memory CD4⁺ and CD8⁺ T_{EM}, T_{CM}, and T_{RM} cells through the multi-epitope/CXCL11 prime/pull Coronavirus vaccine protected HLA-DR*0101/HLA-A*0201/hACE2 triple transgenic mice following SARS-CoV-2 infection: Since the multi-epitope//CXCL11 prime/pull Coronavirus vaccine appeared to prevent weight loss, to reduce virus replication and inflammation in the lungs and to prevent deaths in HLA-DR*0101/HLA-A*0201/hACE2 triple transgenic mice following infection with SARS-CoV-2, next it was determined whether such protection would be associated with increased frequencies of lung-resident memory CD4+ and CD8+ T cells.

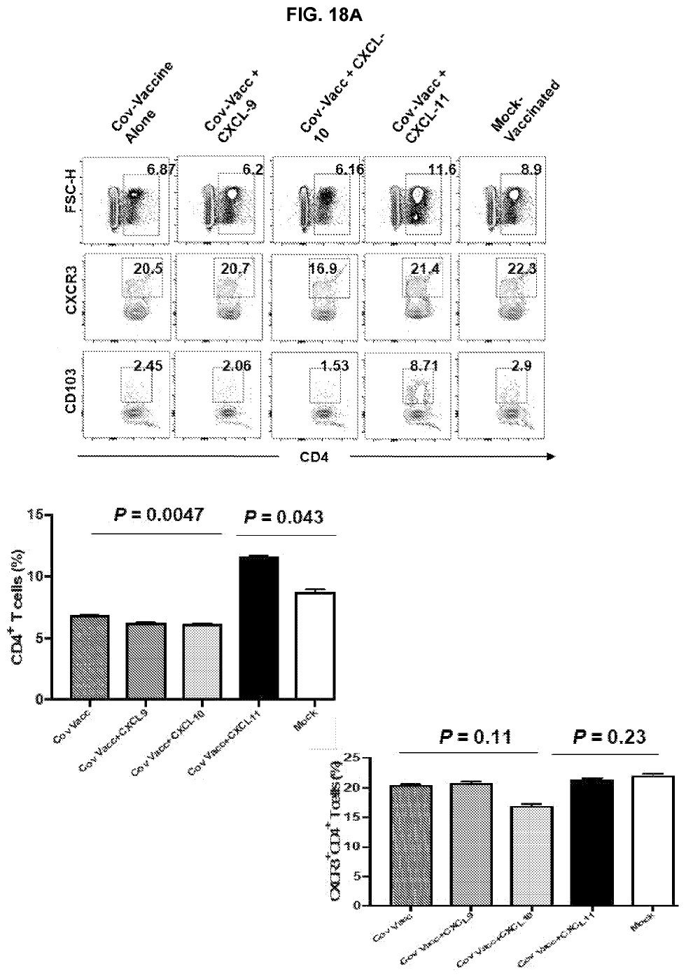

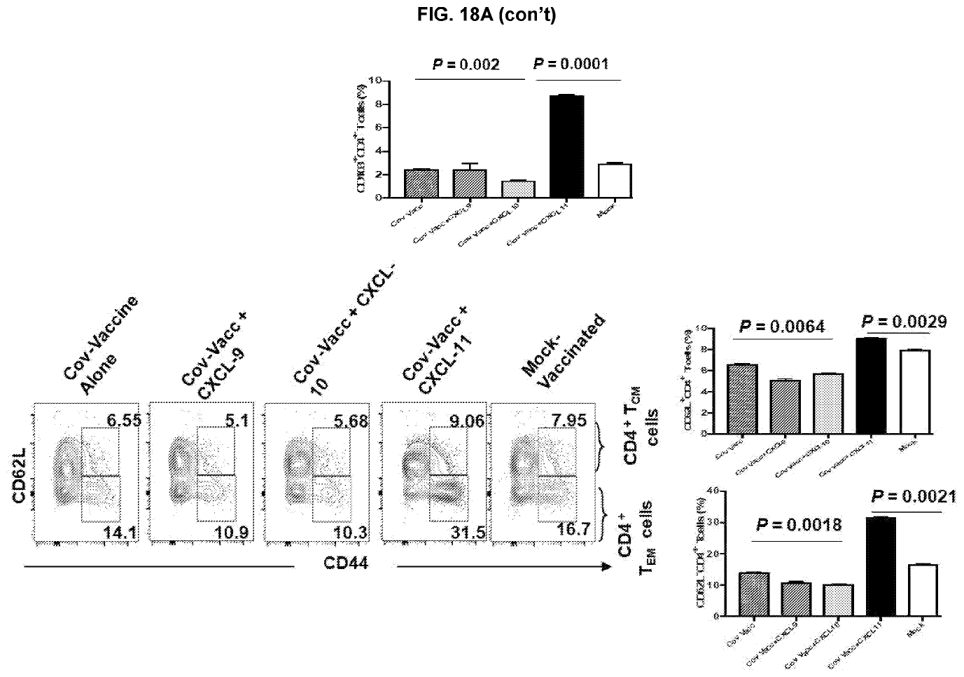

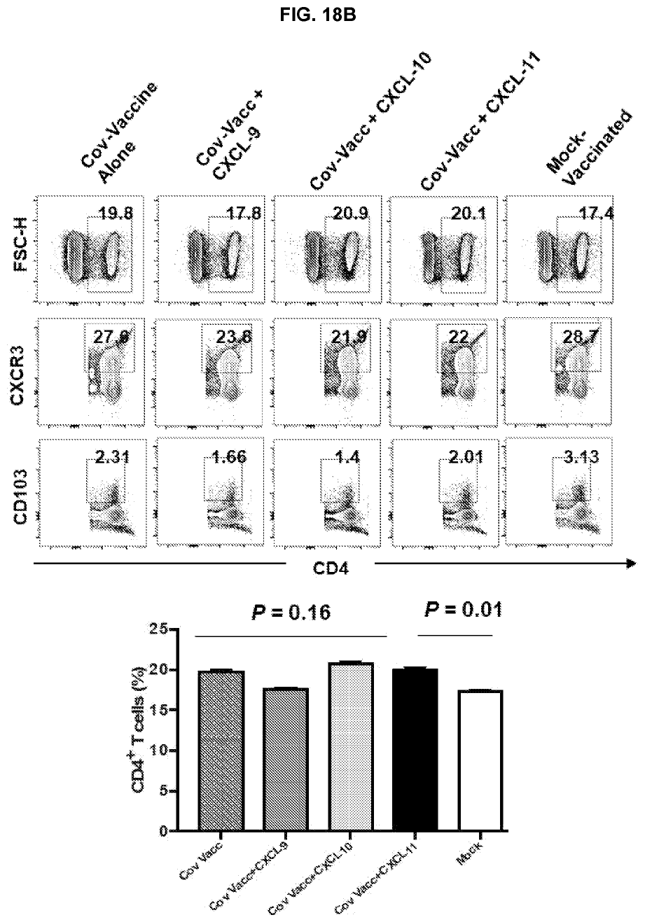

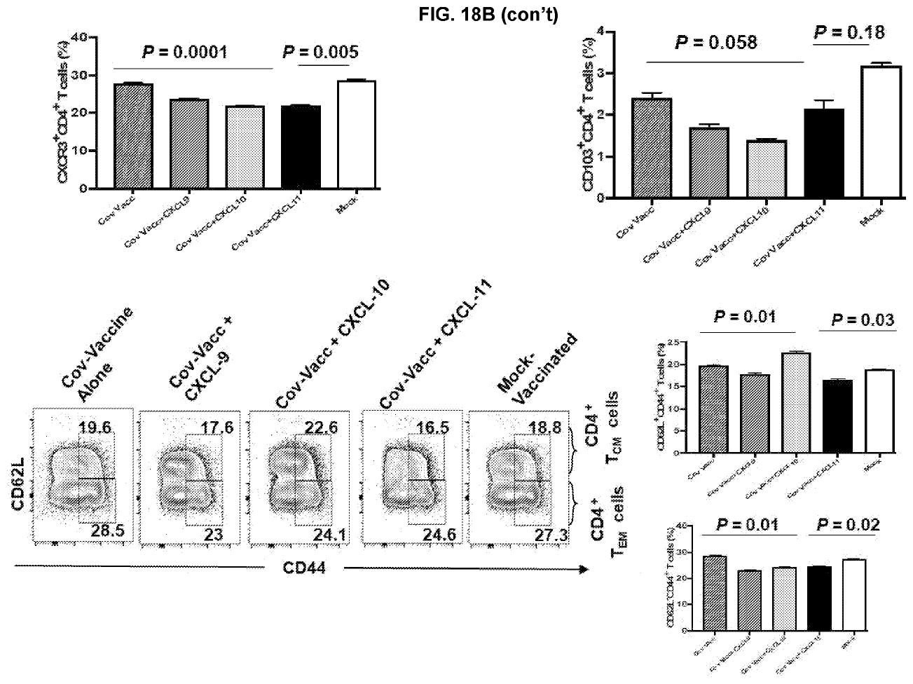

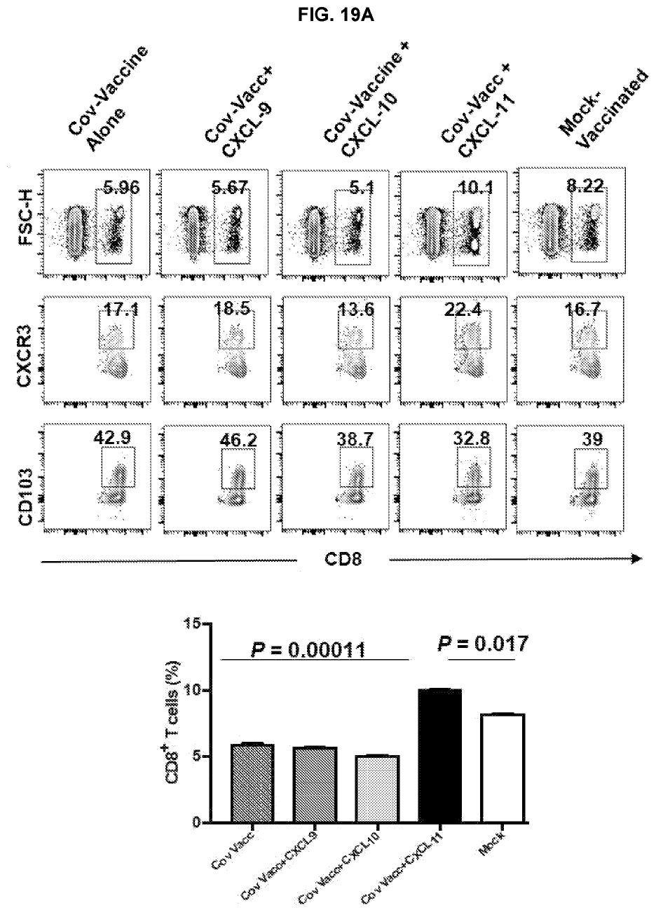

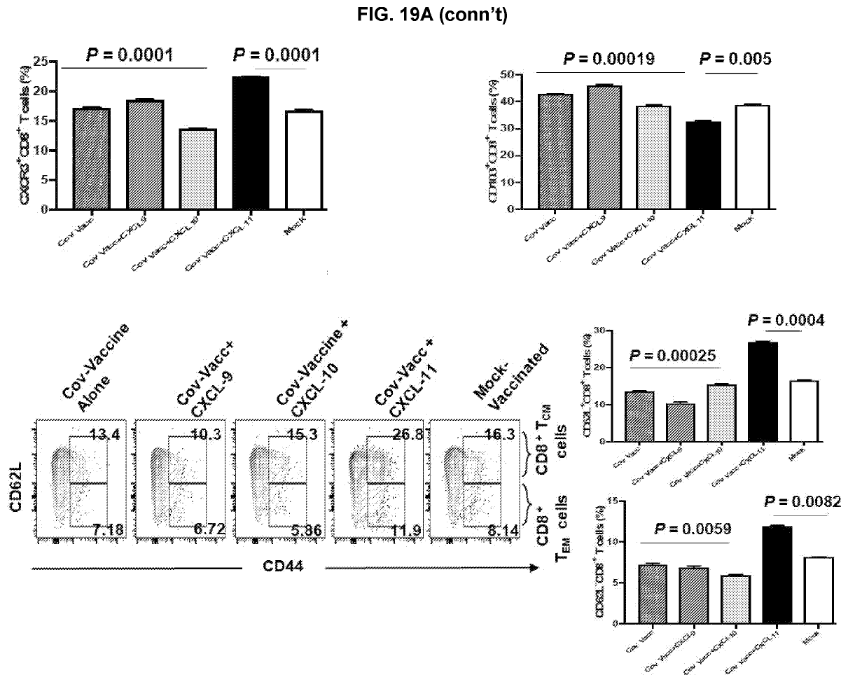

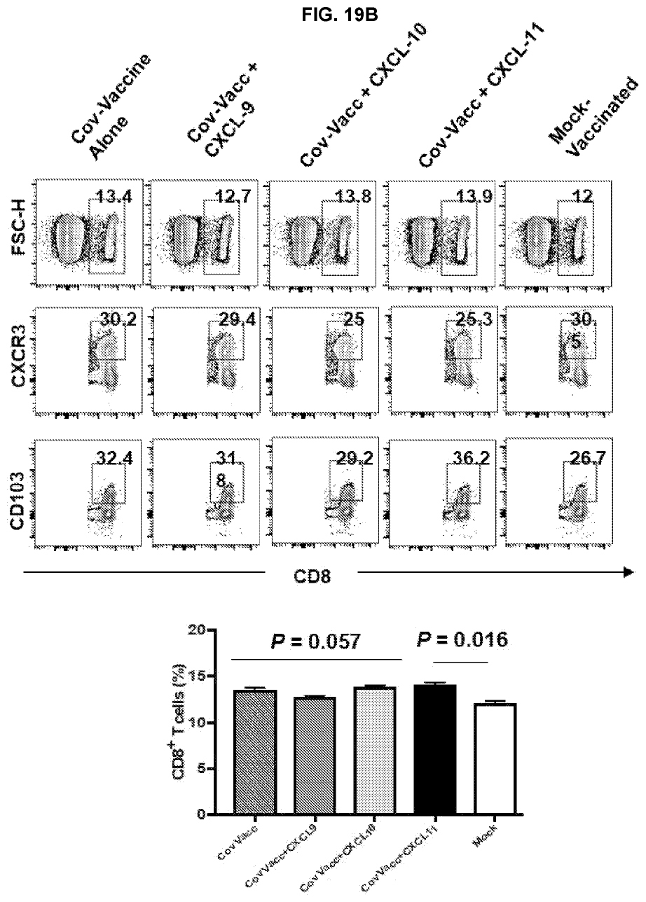

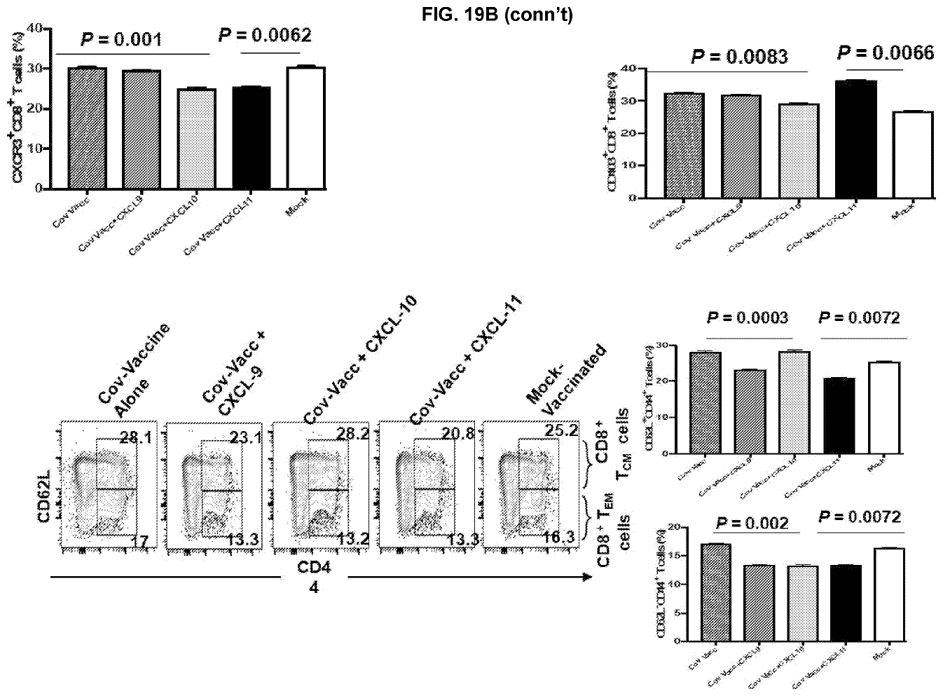

Next, the effect of immunization with the multi-epitope prime/pull Coronavirus vaccine candidate based on CXCL-9, CXCL-10, and CXCL-11 T-cell-attracting chemokines were compared on the frequencies of three major lung-resident memory CD4⁺ and CD8⁺ T cell subsets (i.e., effector memory (T_{EM}), resident memory (T_{RM}). and central memory (T_{CM})) in the lungs of HLA-DR*0101/HLA-A*0201/hACE2 triple transgenic mice, before (FIGS. 18A and 18B) and after (FIGS. 19A and 19B) challenge with SARS-CoV-2 (FIG. 16B). As controls, the frequencies of three major lung-resident memory and CD4+CD8+ T cell subsets were compared in HLA-DR*0101/HLA-A*0201/hACE2 triple transgenic mice that received the same multi-epitope CoV-Vacc bearing human CD4⁺ and CD8⁺ T cell epitopes without chemokine treatment (CoV Vacc alone) as well as in mock-vaccinated mice (Mock).

For this experiment, eight to nine-week-old HLA-DR*0101/HLA-A*0201/hACE2 triple transgenic mice (n=35) were first immunized with the multi-epitope Coronavirus vaccine (CoV-Vacc) delivered intranasally at 2×10¹⁰ VP per mouse. Vaccinated HLA-DR*0101/HLA-A*0201/hACE2 triple transgenic were subsequently left untreated (control) or treated intranasally on days 10, 12, and 14 post-vaccination and on days 22, 24, and 26 post-vaccination with the CXCL-9, CXCL-10, or CXCL-11 T-cell-attracting chemokine. Vaccinated/chemokine-treated and Vaccinated/untreated mice were euthanized on day 27 post-vaccine and the frequencies of the three major lung-resident memory CD4⁺ and CD8⁺ T cells expressing CXCR3, CD103, CD62L, and CD44 among total lung cells (i.e. effector memory (T_{EM}), resident memory (T_{RM}), and central memory (T_{CM})) were determined by FACS.

A significant increase was found in the frequency of total CD4+ T cells among immunized mice compared to mock mice (FIG. 18A). Similarly, a significant increase in CD103⁺CD4⁺ T cells, CD44⁺CD62L CD4⁺ T cells, and CD44⁺CD62L CD4⁺ T cells in mice immunized with the CoV-Vacc was found. However, CXCR3⁺CD4⁺ T cells did not show significant variation for mice immunized with CoV Vacc+CXCL-9, CoV Vacc+CXCL-10, or CoV Vacc+CXCL-11, compared to mice immunized only with Vacc or mock group of mice. In FIG. 19A, a higher magnitude in the frequency of total CD8+ T cells in lung immune cells was shown to be observed in the immunized mice. Further, the mice immunized with CoV-Vacc showed a significant increase in CXCR3⁺CD8⁺ T cells, CD44⁺CD62L CD8⁺ T cells, and CD44⁺CD62L⁺CD8⁺ T cells when compared to the mock group. However, CD103⁺CD8⁺ T cells showed a decrease in the CoV-Vacc+CXCL-11 group compared to the immunized and mock groups.

Altogether, these results demonstrate that the protection induced by the multi-epitope//CXCL11 prime/pull Coronavirus vaccine in SARS-CoV-2 infected HLA-DR*0101/HLA-A*0201/hACE2 triple transgenic mice are associated with high frequencies of lung-resident memory CD4⁺ and CD8⁺ T_{EM}, T_{CM}, and T_{RM} cells.

Bolstering the function of virus-specific lung-resident memory CD4⁺ and CD8⁺ T cells through the multi-epitope/CXCL11 prime/pull Coronavirus vaccine protected HLA-DR*0101/HLA-A*0201/hACE2 triple transgenic mice following SARS-CoV-2 infection: Since the protection induced by the multi-epitope//CXCL11 prime/pull Coronavirus vaccine in SARS-CoV-2 infected HLA-DR*0101/HLA-A*0201/hACE2 triple transgenic mice is associated with high frequencies of lung-resident memory CD4⁺ and CD8⁺ T_{EM}, T_{CM}, and T_{RM} cells, we next determined whether such protection would be associated with increased frequencies of lung-resident memory CD4⁺ and CD8⁺ T cells.

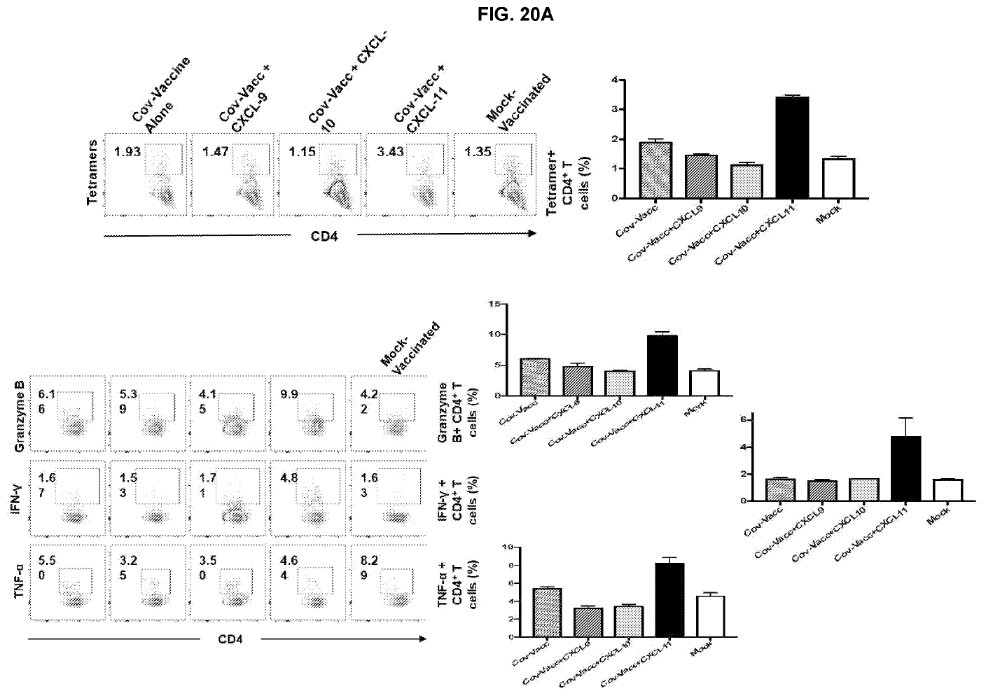

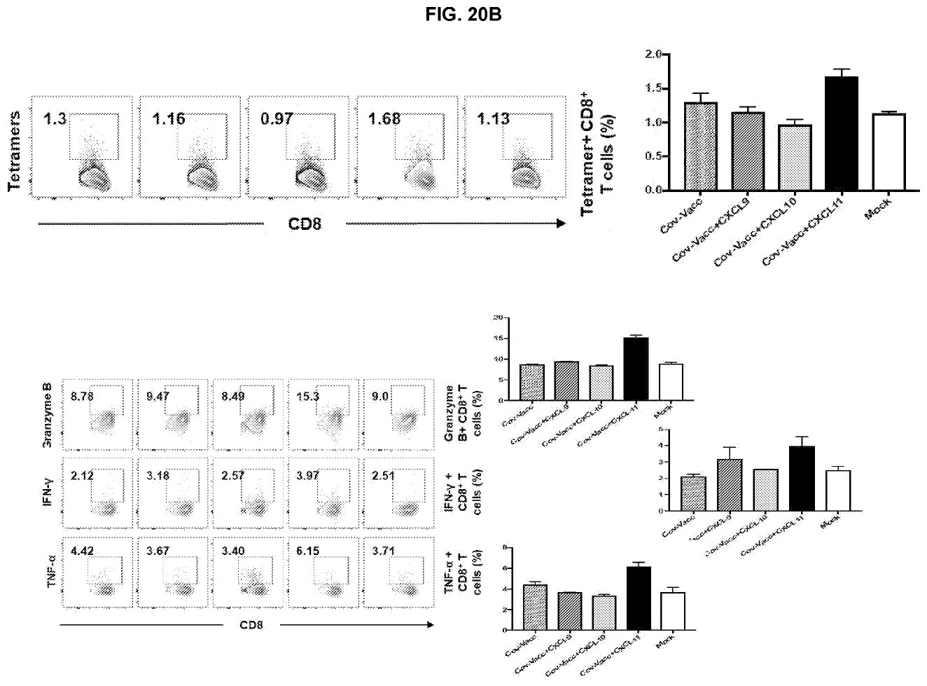

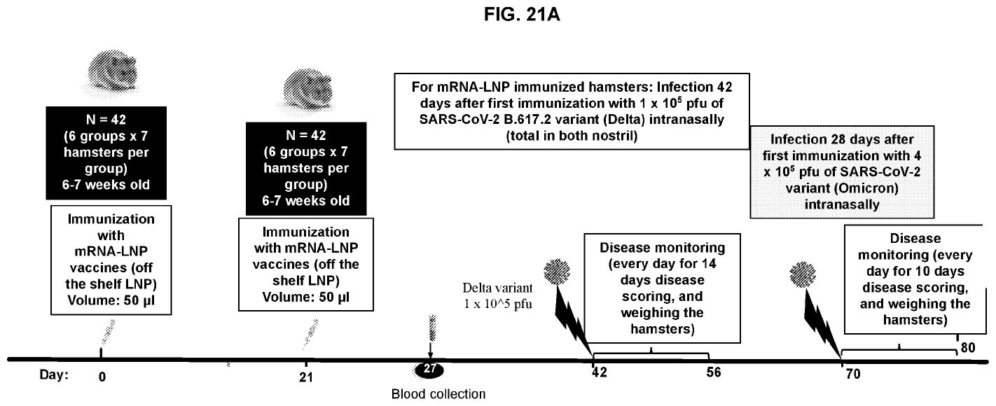

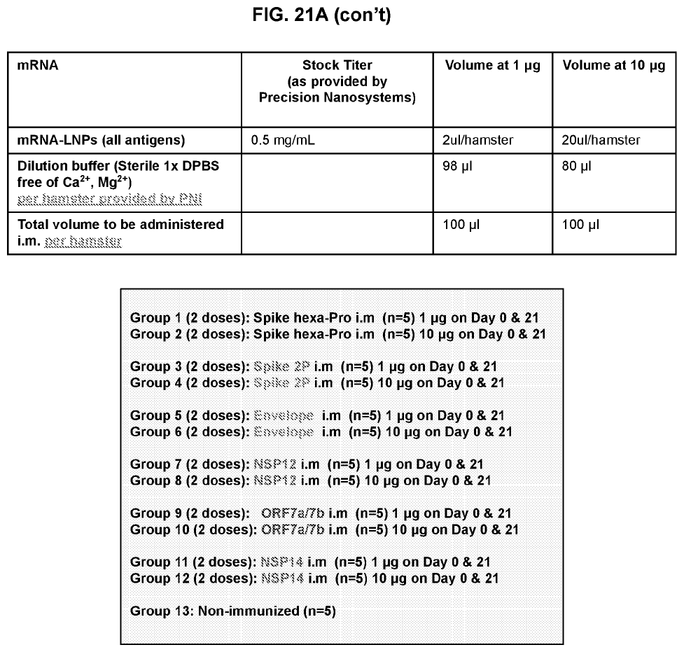

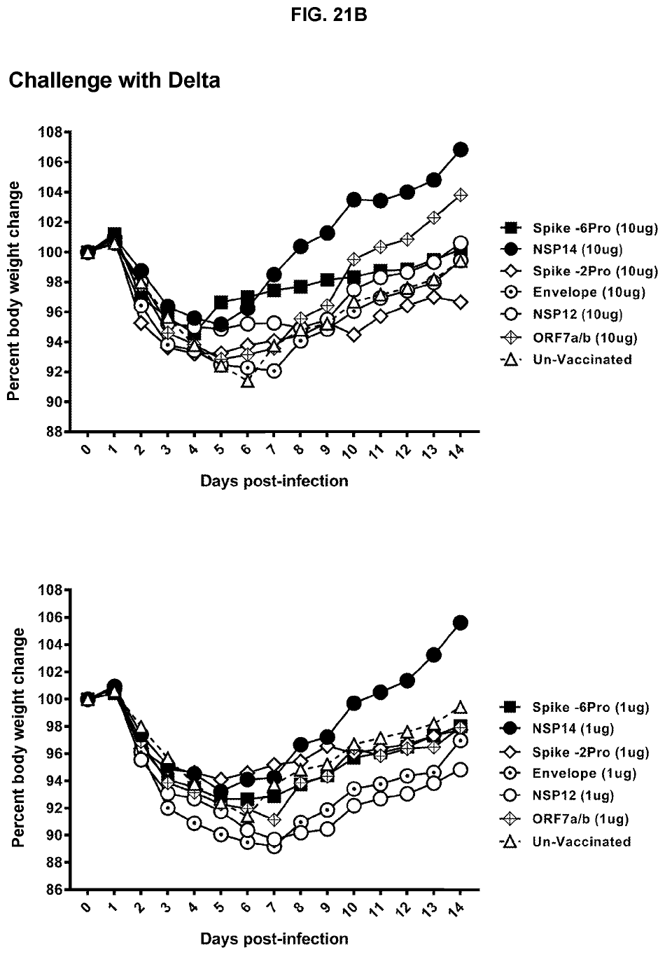

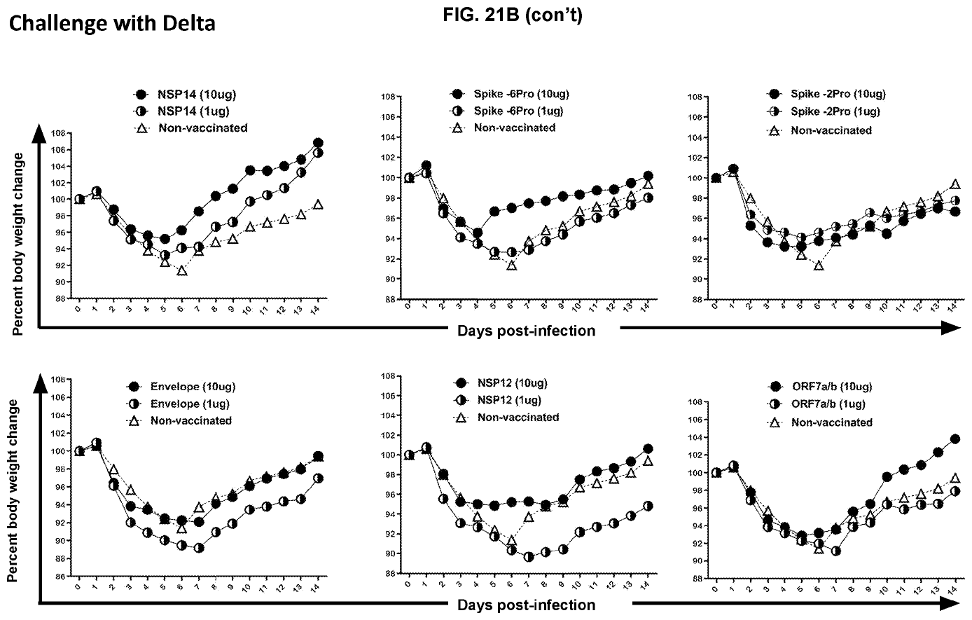

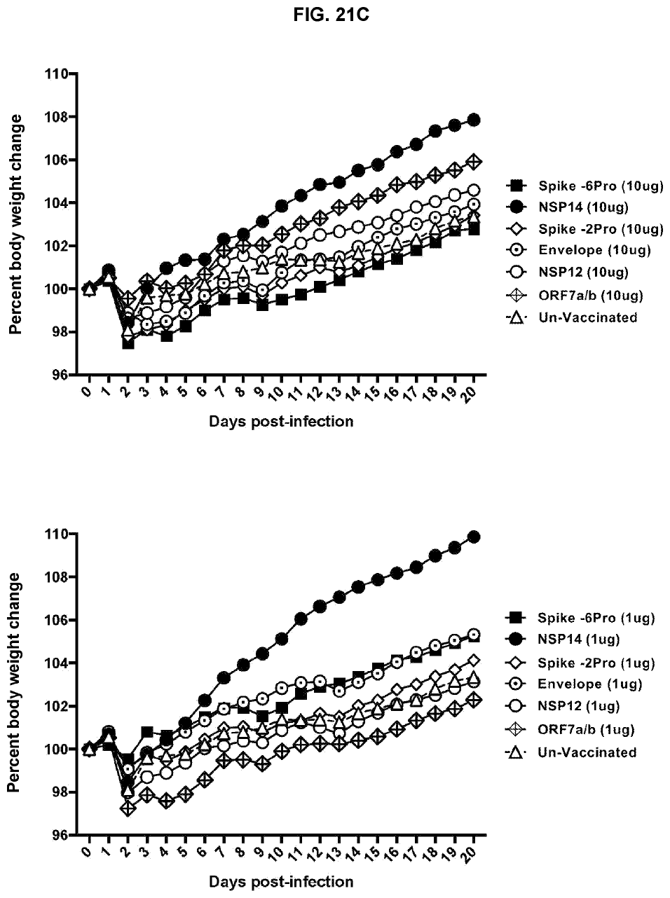

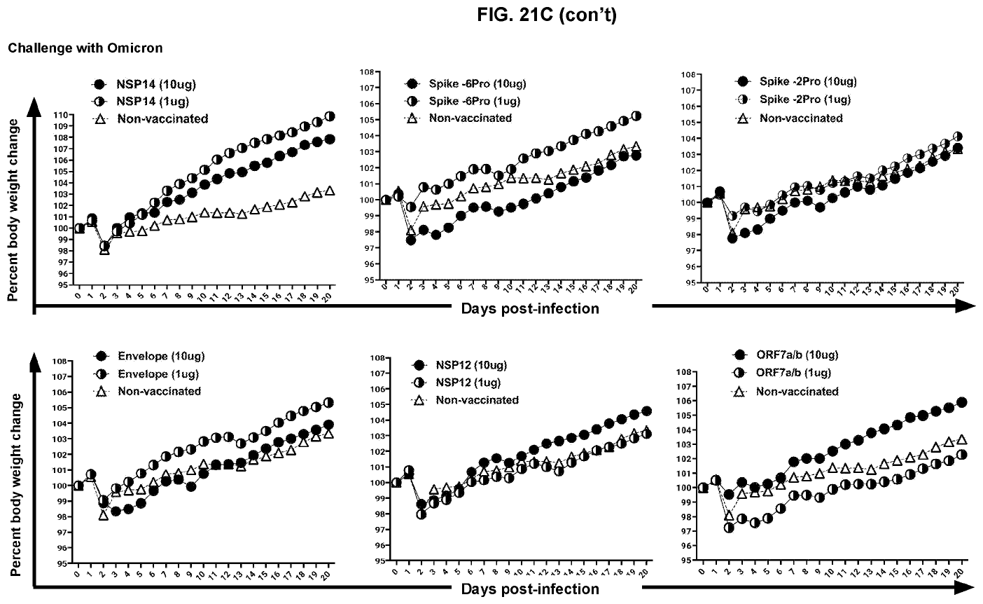

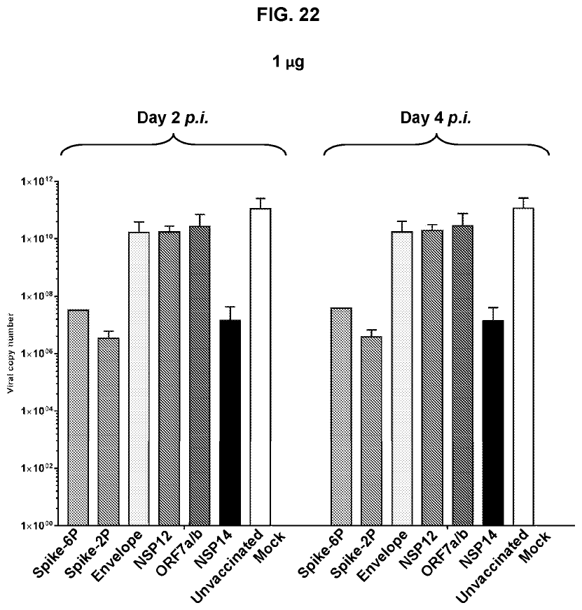

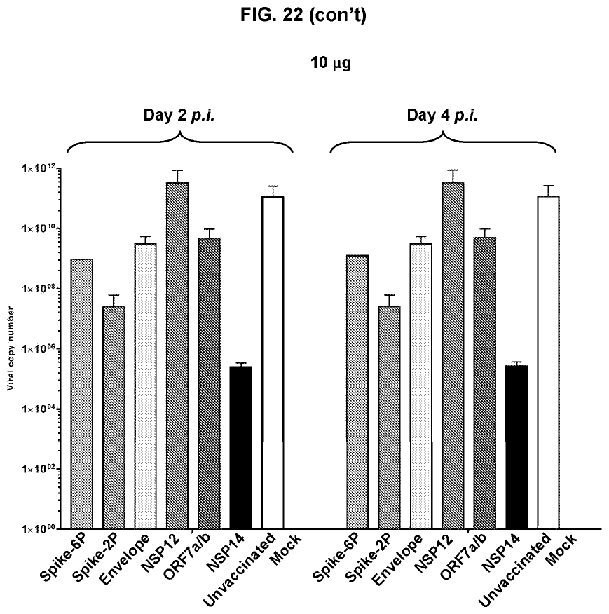

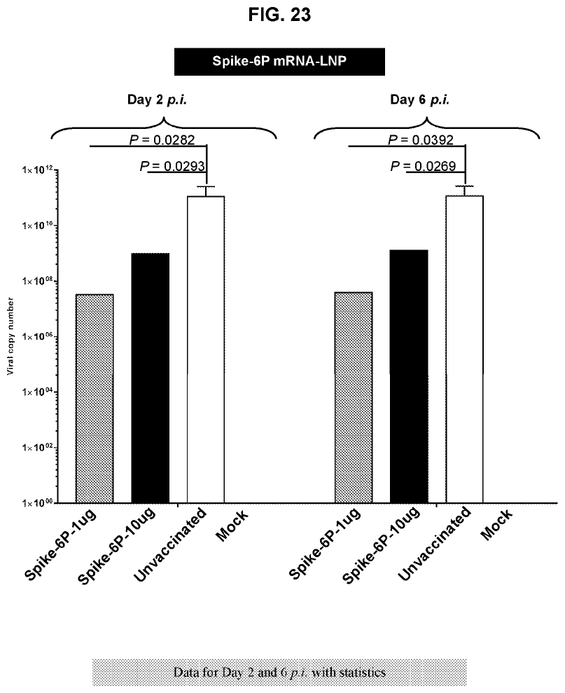

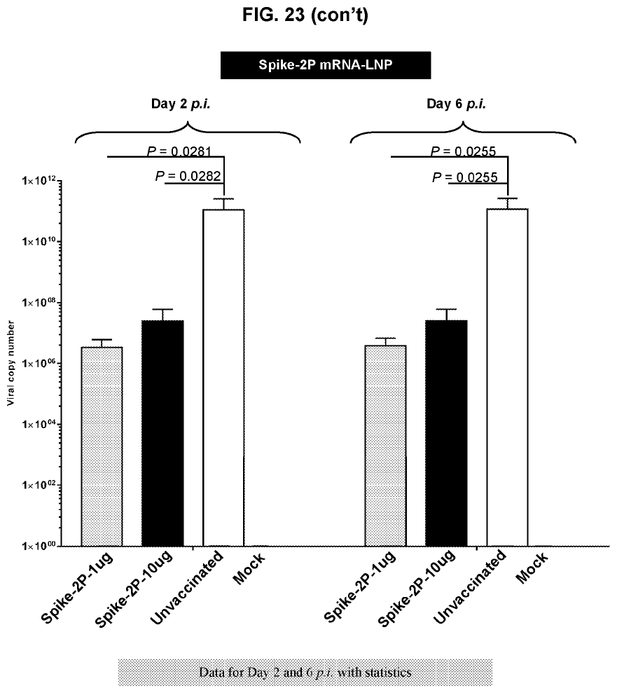

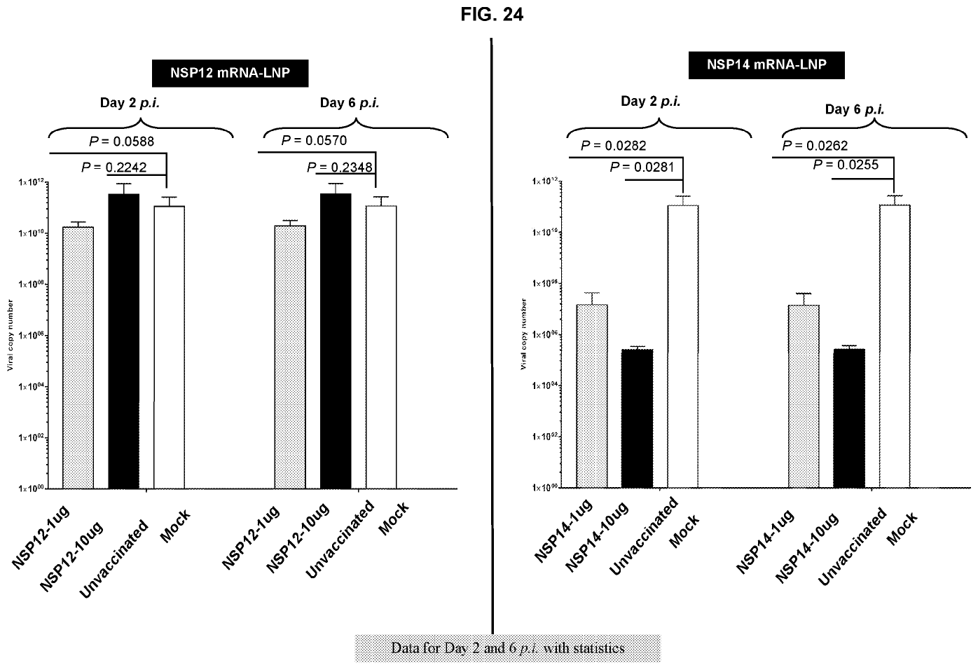

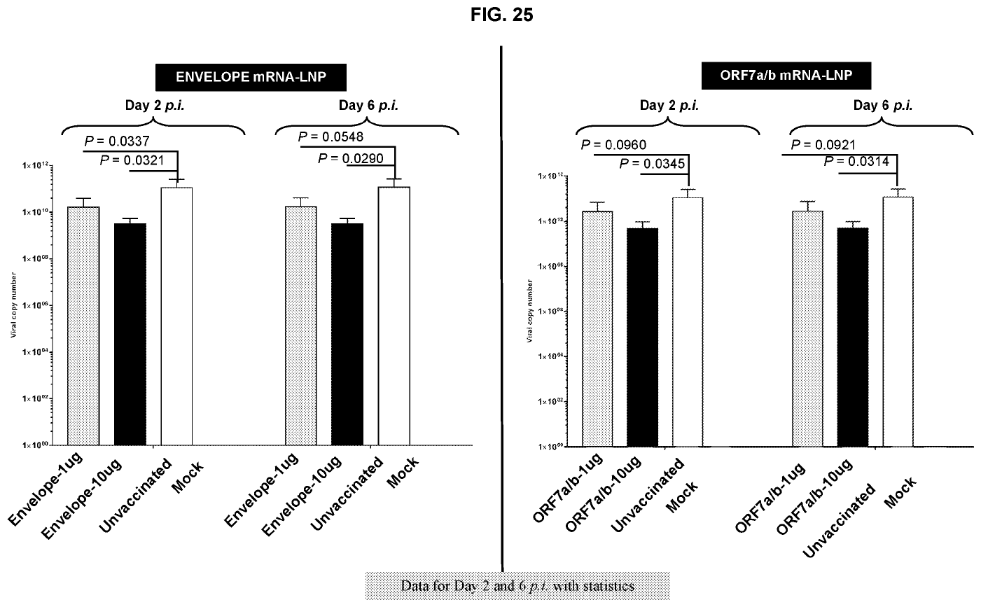

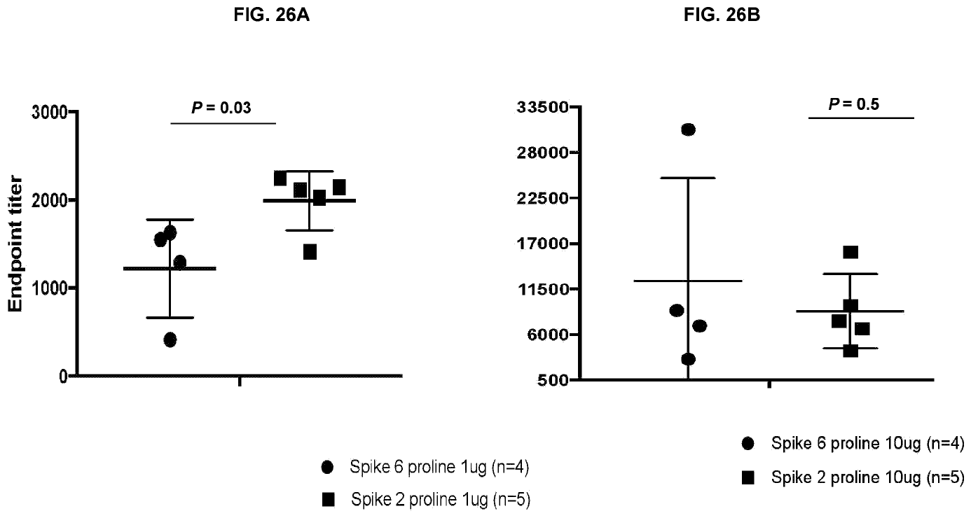

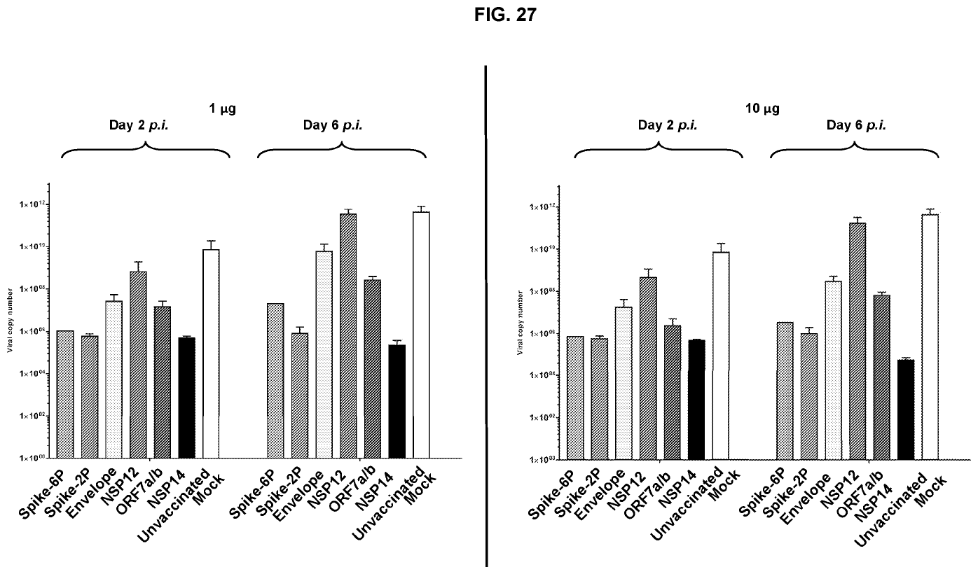

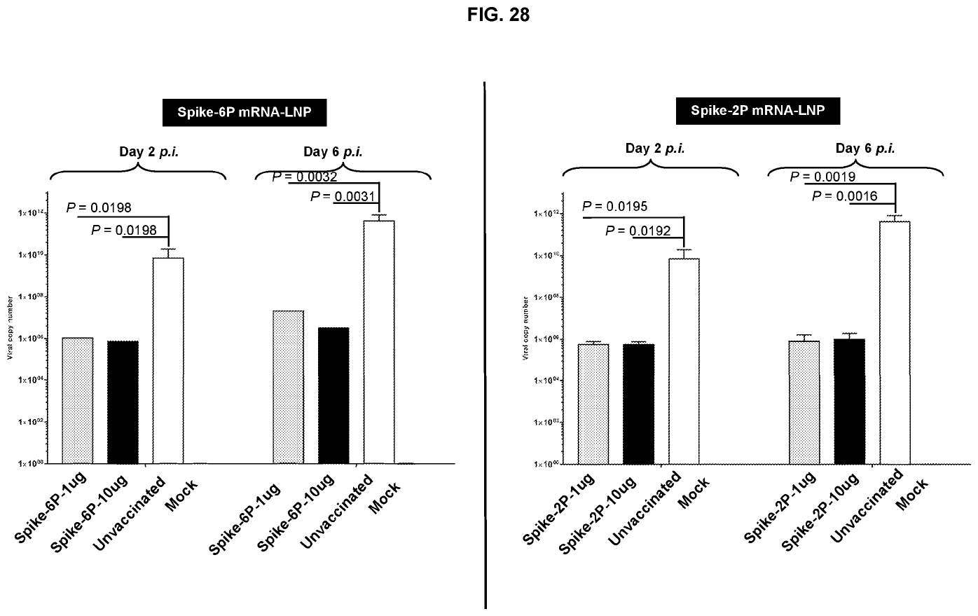

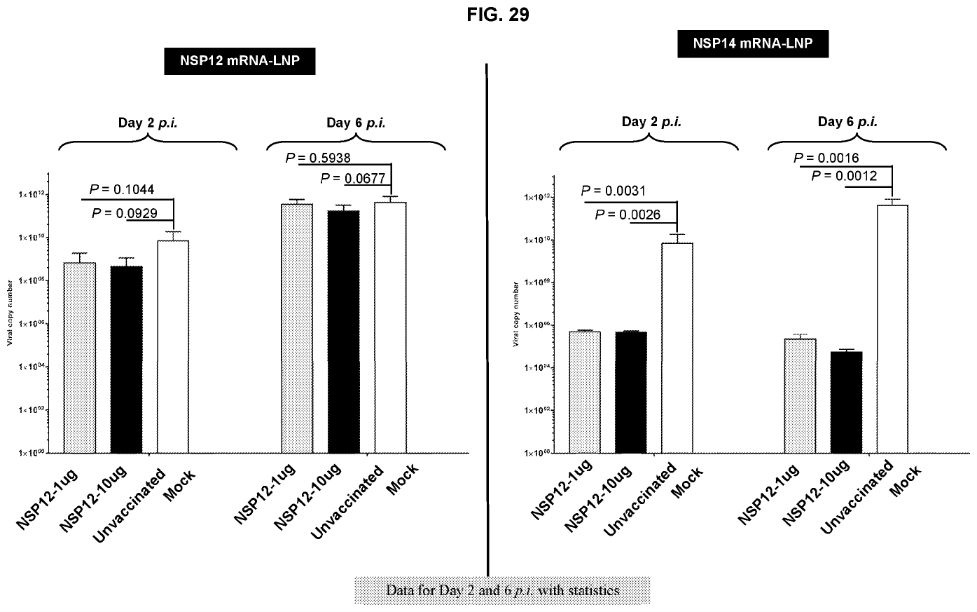

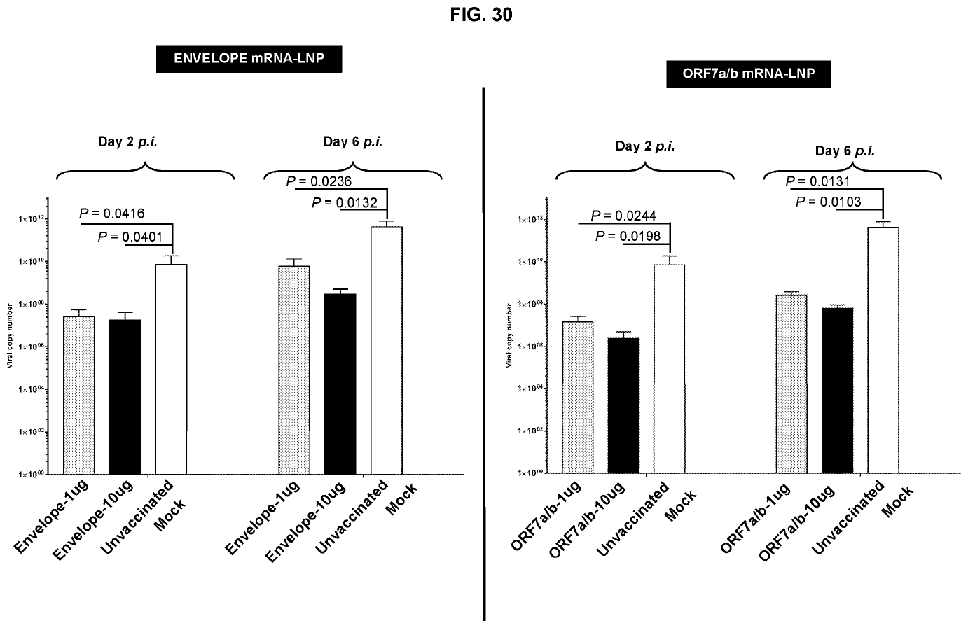

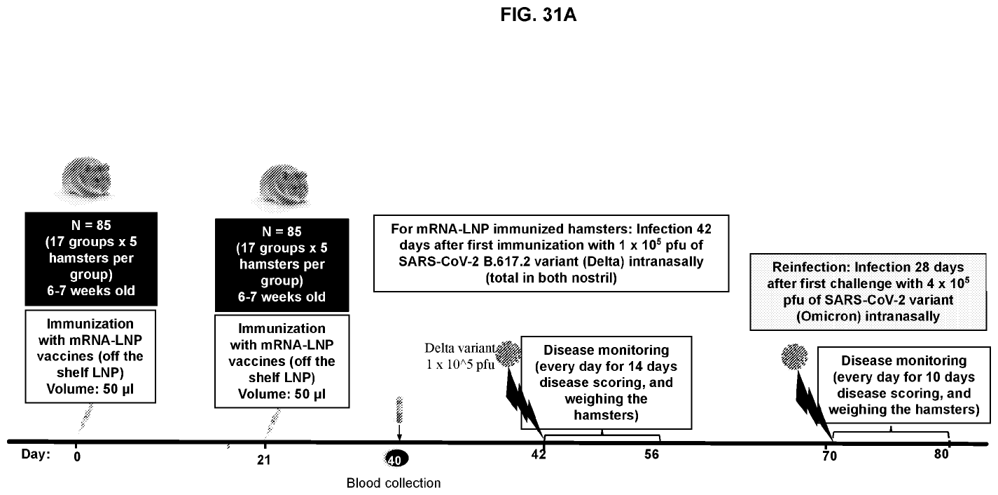

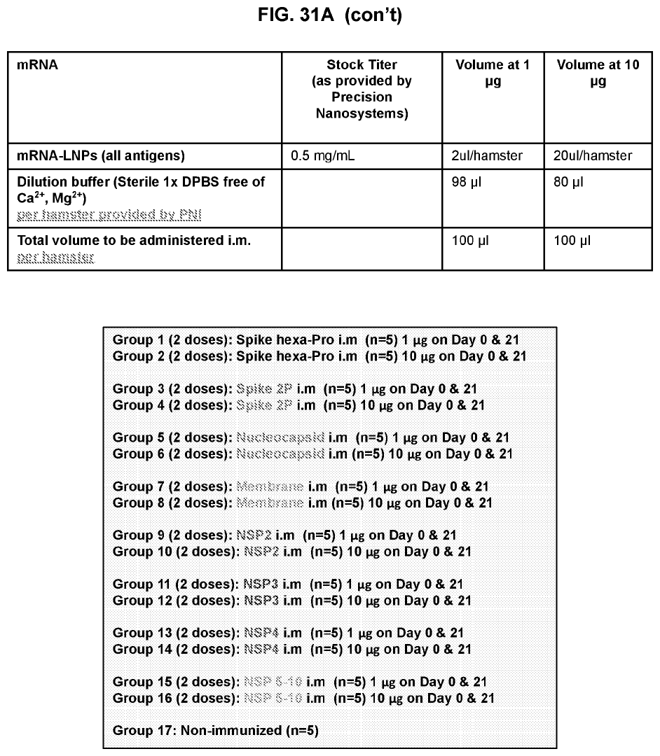

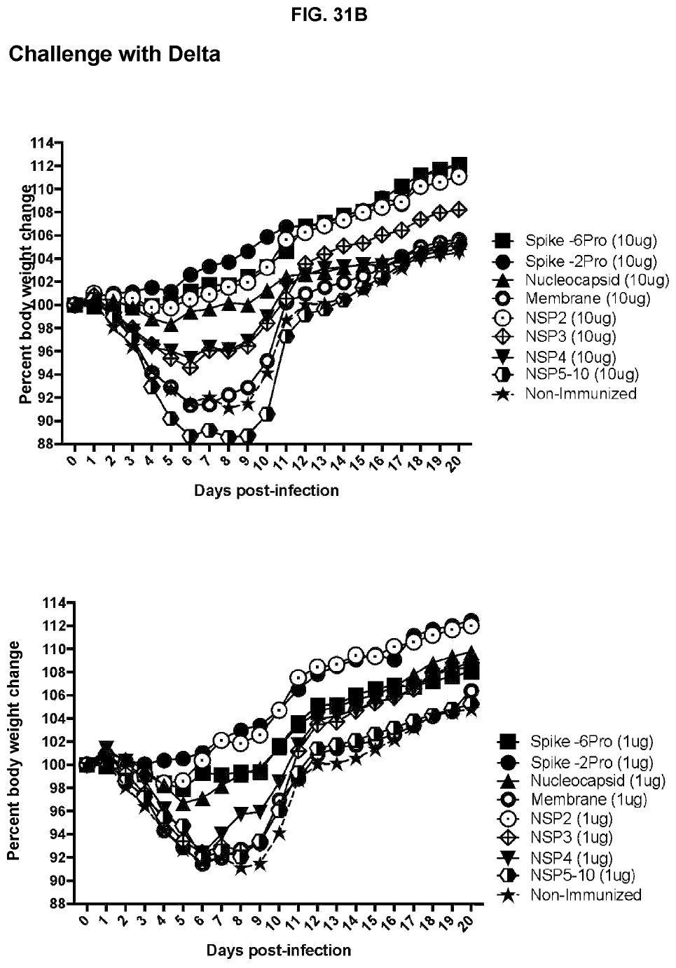

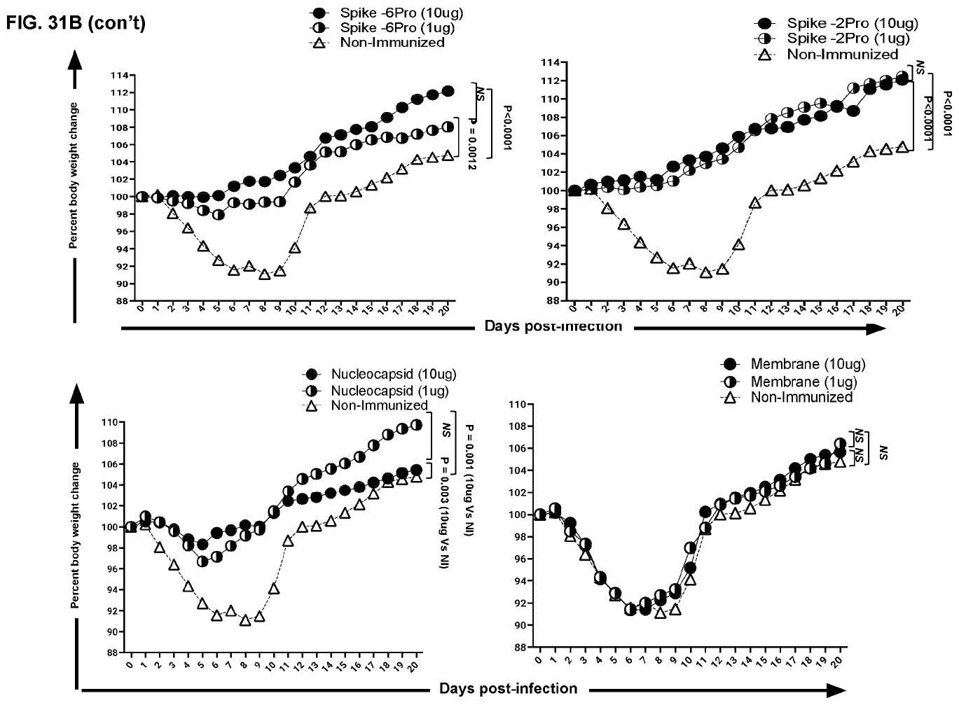

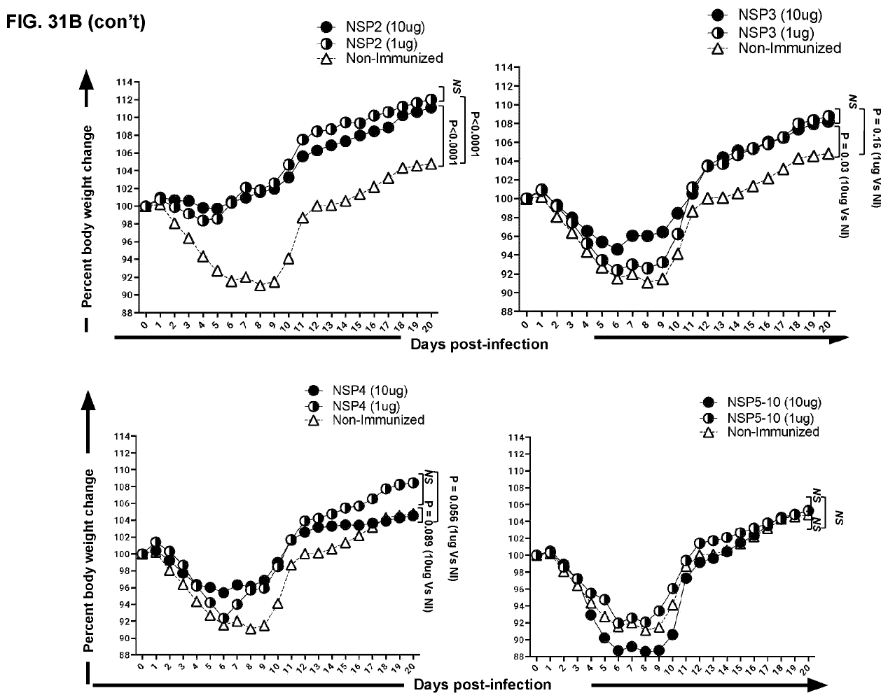

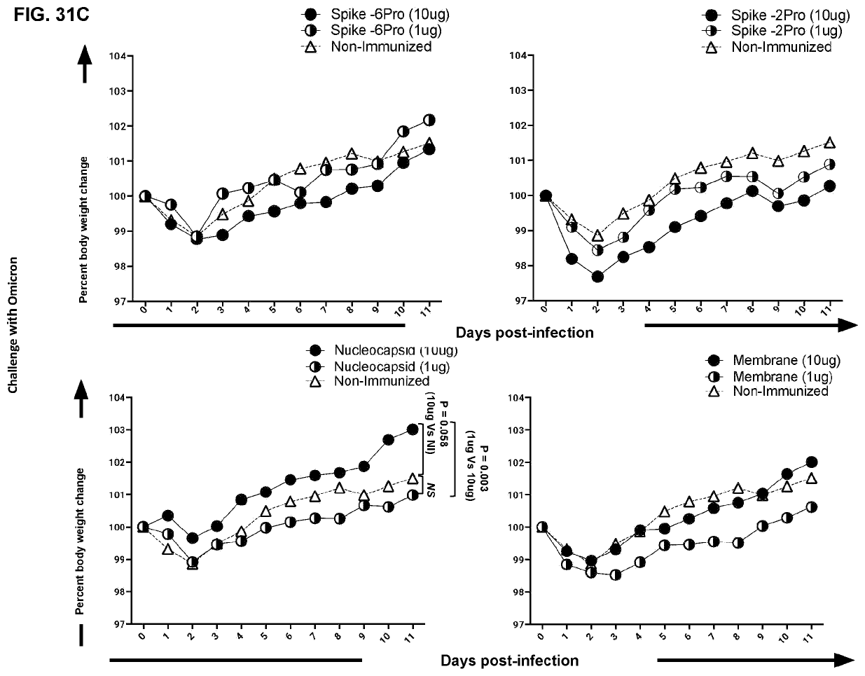

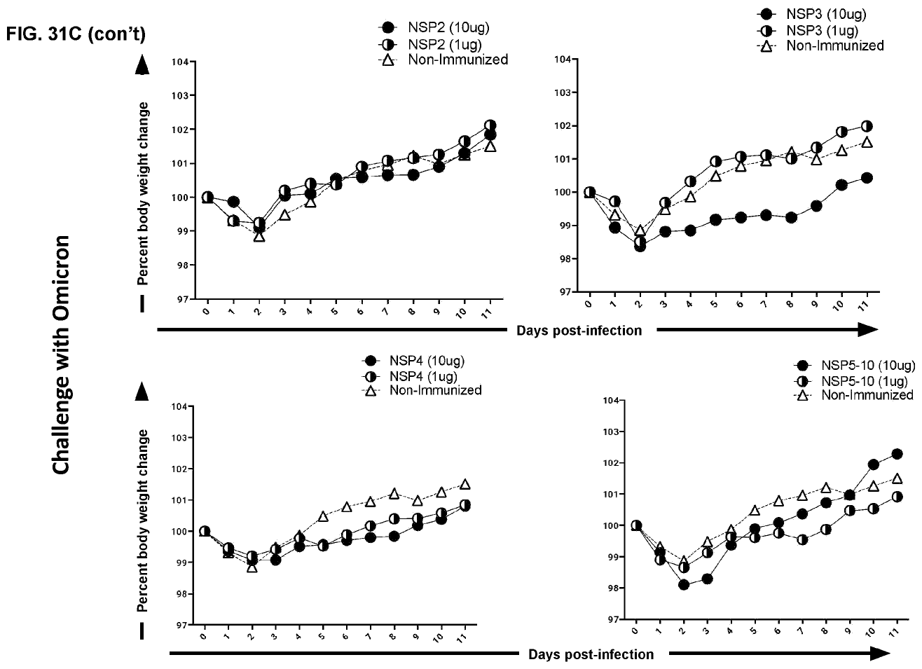

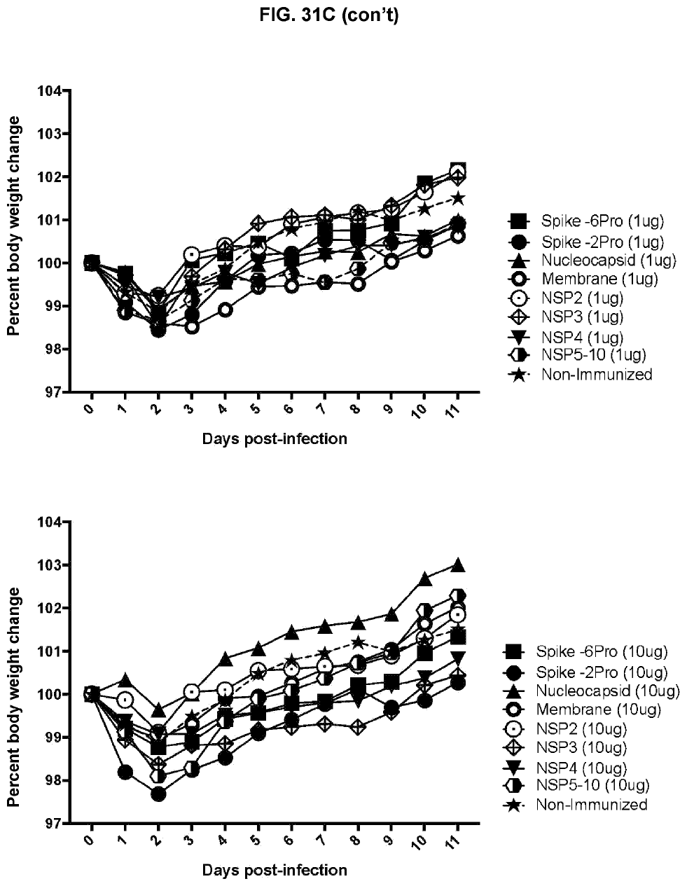

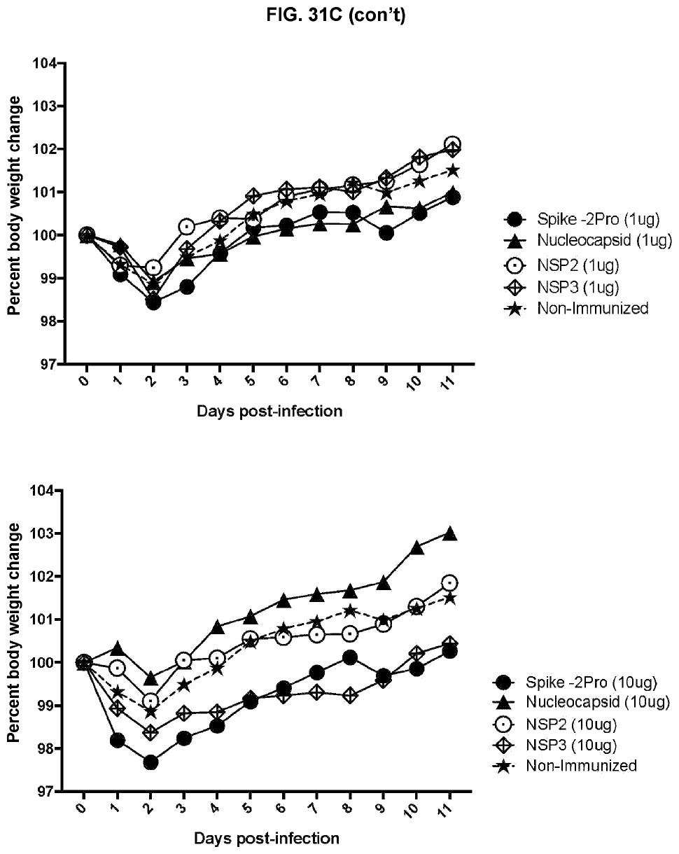

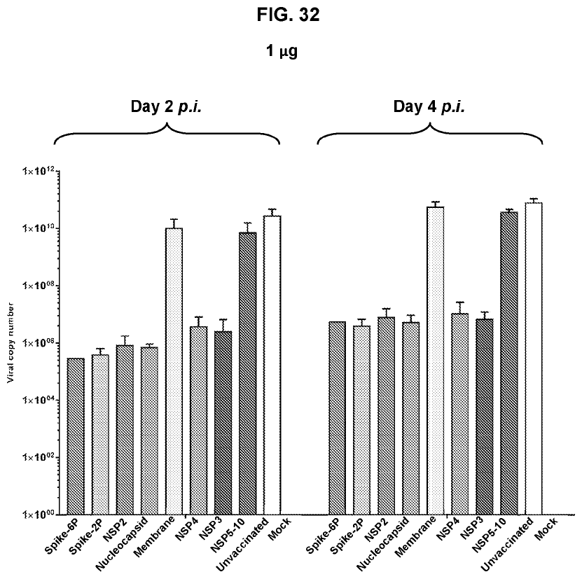

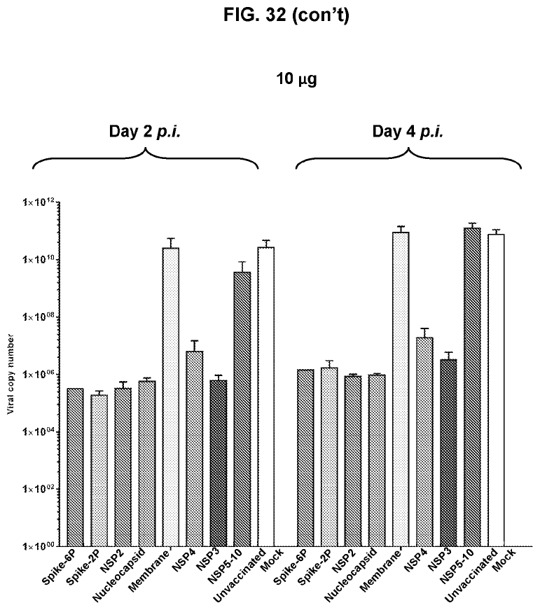

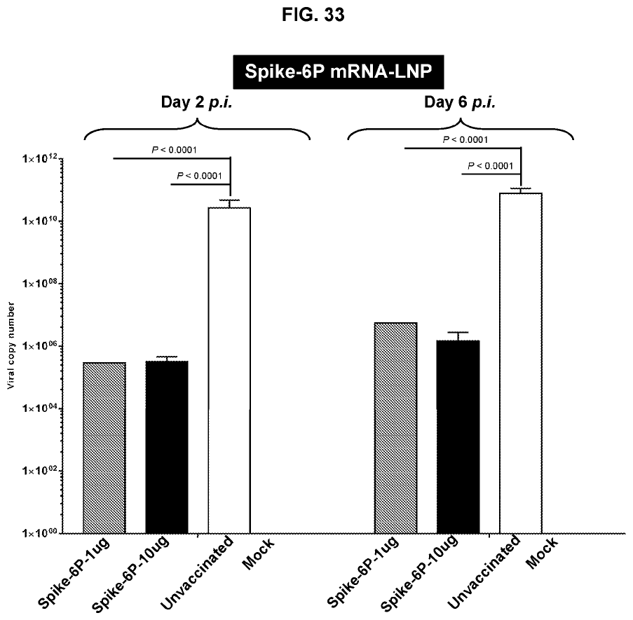

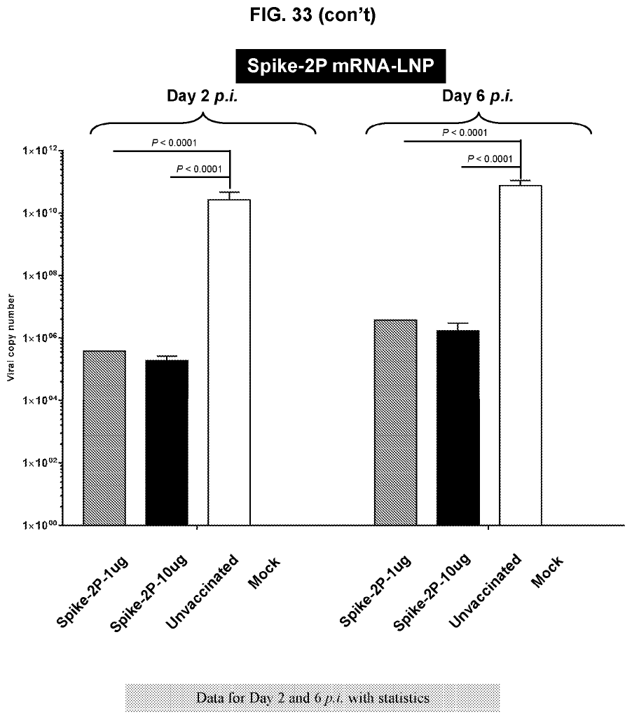

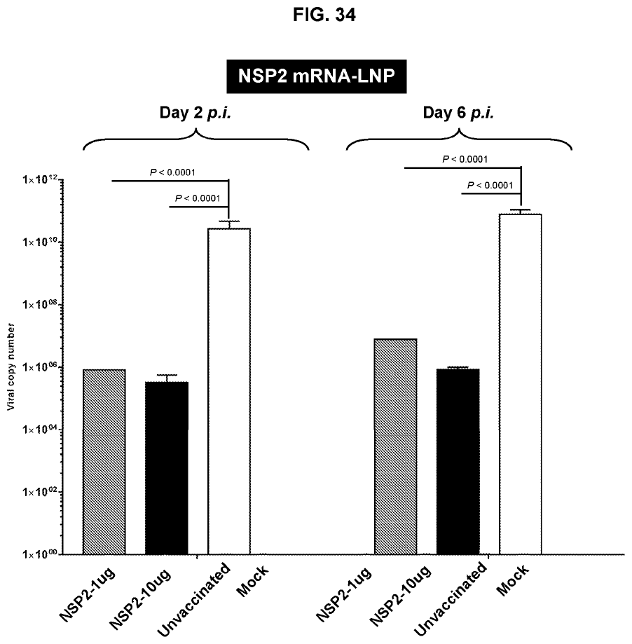

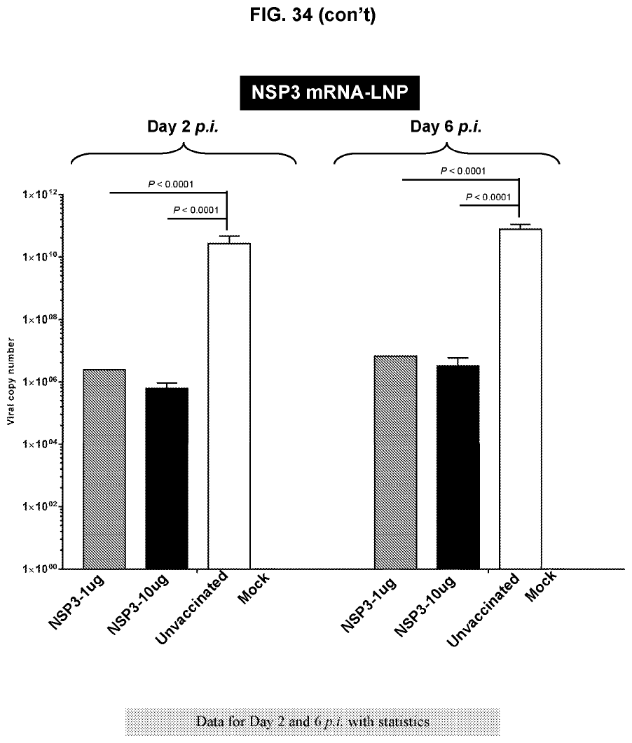

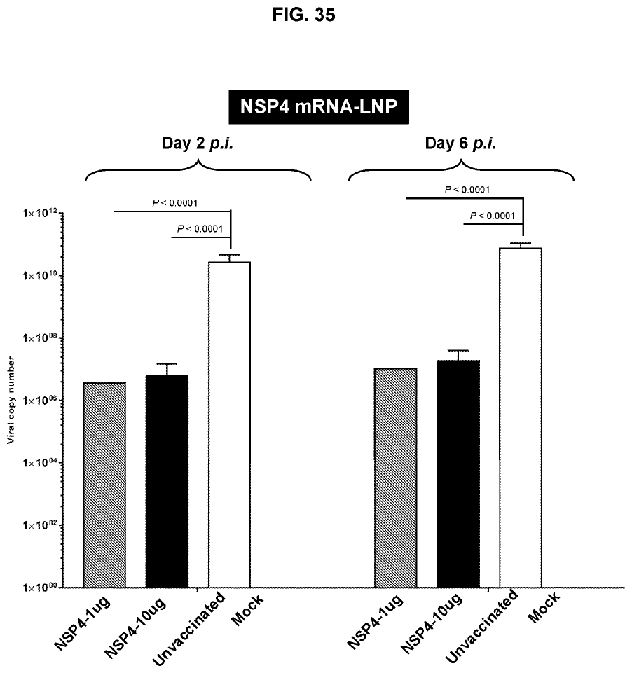

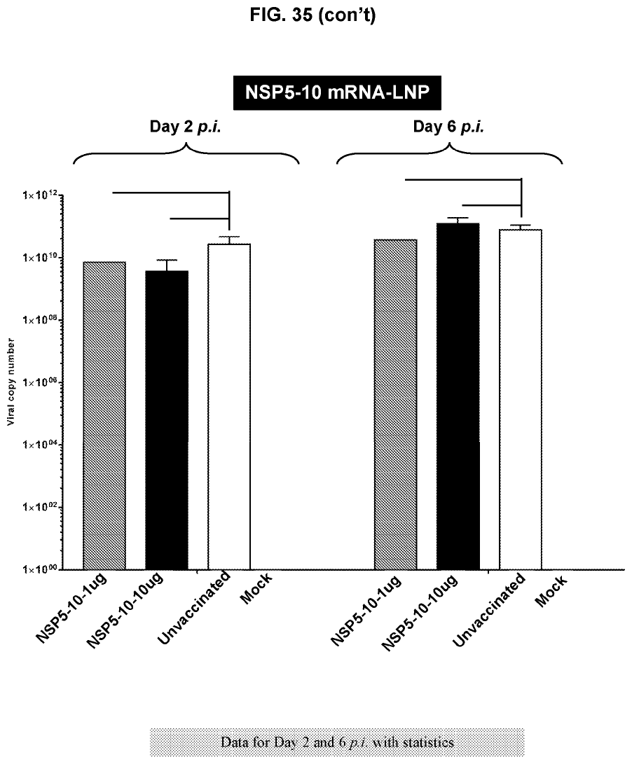

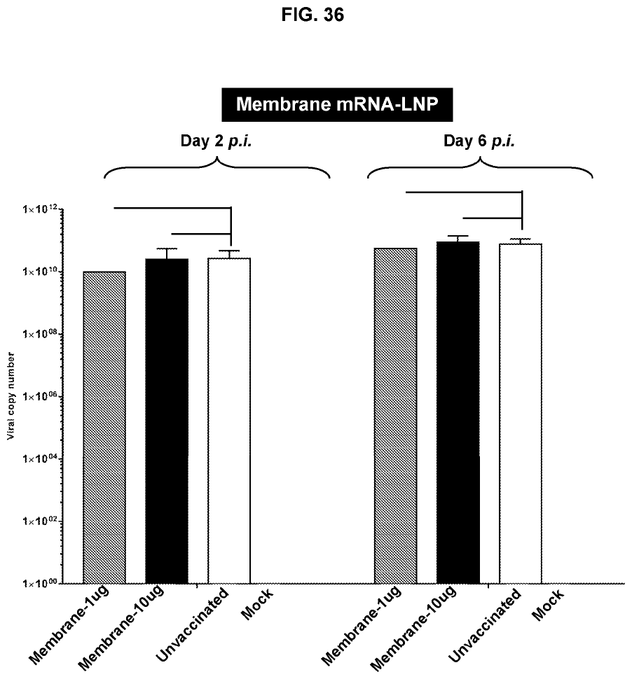

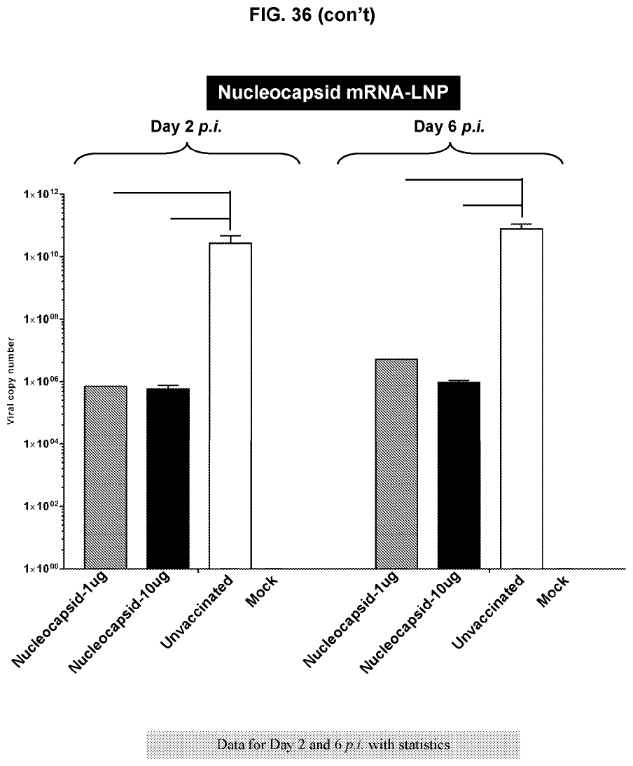

The effect of immunization was compared with the three multi-epitope prime/pull Coronavirus vaccine candidates based on CXCL-9, CXCL-10, and CXCL-11 T-cell-attracting chemokines on the function and specificity of pulled CD4⁺ and CD8⁺ T cells in the lungs of HLA-DR*0101/HLA-A*0201/hACE2 triple transgenic mice, before (FIGS. 20A and 20B) challenge with SARS-CoV-2 (FIG. 16B). As controls, HLA-DR*0101/HLA-A*0201/hACE2 triple transgenic mice that received the same multi-epitope CoV-Vacc bearing human CD4⁺ and CD8⁺ T cell epitopes without chemokine treatment (CoV Vacc alone) as well as in mock-vaccinated mice (Mock).

For this experiment, eight to nine-week-old HLA-DR*0101/HLA-A*0201/hACE2 triple transgenic mice (n=35) were first immunized with the multi-epitope Coronavirus vaccine (CoV-Vacc) delivered intranasally at 2×10¹⁰ VP per mouse. Vaccinated HLA-DR*0101/HLA-A*0201/hACE2 triple transgenic were subsequently left untreated (control) or treated intranasally on days 10, 12, and 14 post-vaccination and on days 22, 24, and 26 post-vaccination with the CXCL-9, CXCL-10, or CXCL-11 T-cell-attracting chemokine. Vaccinated/chemokine-treated and Vaccinated/untreated mice were euthanized on day 27 post-vaccine immune cells were harvested for the frequency and the function of major lung-memory CD4⁺ and CD8⁺ T cells determined by FACS.

A significant increase in the percentage of tetramer⁺CD4⁺ T cells was found among immunized and CXCL11-treated mice compared to all other mice groups (FIG. 20A). Similarly, we found a significant increase in Granzyme B⁺CD4⁺ T cells, IFN-γ⁺CD4⁺ T cells, and TNF-α⁺CD4⁺ T cells in mice immunized with the CoV-Vacc and treated with the chemokine CXCL11. In FIG. 20B, a slight increase in magnitude in the percentage of tetramer⁺CD8⁺ T cells in the lungs of immunized and CXCL11-treated mice compared to all other mice groups. A significant increase in Granzyme B⁺CD8⁺ T cells and TNF-α⁺CD8⁺ T cells was shown in the lungs of the CoV Vacc+CXCL-11 group compared to the immunized and mock groups. While IFN-γ⁺CD8⁺ T cells didn't show a significant increase among all the groups.

Altogether, these results demonstrate that the protection induced by the multi-epitope//CXCL11 prime/pull Coronavirus vaccine in SARS-CoV-2 infected HLA-DR*0101/HLA-A*0201/hACE2 triple transgenic mice are associated with high frequencies of specific of CD4⁺ and CD8⁺ T cells in the lungs. As well as high expression of Granzyme B, IFN-γ, and TNF-α of those cells.

Mice: Female K18-hACE2 transgenic mice (8-9 weeks old) were purchased from the Jackson Laboratory (Bar Harbor, ME). K18-hACE2 mice breeding was conducted in the UCI animal facility where female mice were used at 8-9 weeks. In addition, female HLA-DR*0101/HLA-A*0201/hACE2 triple transgenic mice (8-9 weeks old) were used. The HLA-DR*0101/HLA-A*0201/hACE2 triple transgenic mouse colony was established here at the UCI by cross-breeding K18-hACE2 mice (51) with double transgenic HLA-DR*0101/HLA-A*0201 mice (17). The animal studies were performed at the University of California Irvine and adhered to the Guide for the Care and Use of Laboratory Animals published by the US National Institute of Health. All animal experiments were performed under the approved IACUC protocol #AUP-22-086.

Immunization and CXC chemokine treatment: Groups of (8-9 weeks old) female HLA-DR*0101/HLA-A*0201/hACE2 triple transgenic mice were immunized intranasally on day 0 with (2×10¹⁰ VP per mouse, n=28) of a multi-epitope Coronavirus vaccine (CoV Vacc), consisting of highly conserved and immunogenic 16 CD8+ T cell epitopes, 6 CD4+ T cell epitopes, and 9 B cell epitopes. As a negative control, mice received sterile PBS (mock). Mice were treated intranasally with CXCL-9, CXCL-10, and CXCL-11 (2.4 μg in 20 μl of sterile PBS/mice). murine MIG (CXCL-9), IP-10 (CXCL-10) and I-TAC (CXCL-11) were obtained (PEPROTECH, USA).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-30. (canceled)

31. A pre-emptive pan-coronavirus vaccine composition, the composition comprising, or comprising a sequence encoding: at least two conserved coronavirus antigens selected from: (i) a conserved coronavirus Spike protein; (ii) a conserved coronavirus NSP2 protein; (iii) a conserved coronavirus NSP3 protein; (iv) a conserved coronavirus NSP14 protein; and (v) a conserved coronavirus Nucleoprotein.

32. The composition of claim 31, wherein the at least two conserved coronavirus antigens are: a conserved coronavirus Spike protein; and a conserved coronavirus NSP2 protein.

33. The composition of claim 31, wherein the at least two conserved coronavirus antigens are: a conserved coronavirus Spike protein; and a conserved coronavirus NSP3 protein.

34. The composition of claim 31, wherein the at least two conserved coronavirus antigens are: a conserved coronavirus Spike protein; and a conserved coronavirus NSP14 protein.

35. The composition of claim 31, wherein the at least two conserved coronavirus antigens are: a conserved coronavirus Spike protein; and a conserved coronavirus Nucleoprotein.

36. (canceled)

37. The composition of claim 31, wherein the at least two conserved coronavirus antigens are: a conserved coronavirus Spike protein; a conserved coronavirus NSP2 protein; and a conserved coronavirus NSP14 protein.

38. The composition of claim 31, wherein the at least two conserved coronavirus antigens are: a conserved coronavirus Spike protein; a conserved coronavirus NSP2 protein; and a conserved coronavirus Nucleoprotein.

39.-40. (canceled)

41. The composition of claim 31, wherein the at least two conserved coronavirus antigens are: a conserved coronavirus Spike protein; a conserved coronavirus NSP14 protein; and a conserved coronavirus Nucleoprotein.

42.-43. (canceled)

44. The composition of claim 31, wherein the at least two conserved coronavirus antigens are: a conserved coronavirus NSP2 protein; and a conserved coronavirus NSP14 protein.

45. The composition of claim 31, wherein the at least two conserved coronavirus antigens are: a conserved coronavirus NSP2 protein; and a conserved coronavirus Nucleoprotein.

46.-47. (canceled)

48. The composition of claim 31, wherein the at least two conserved coronavirus antigens are: a conserved coronavirus NSP14 protein; and a conserved coronavirus Nucleoprotein.

49. (canceled)

50. The composition of claim 31, wherein the at least two conserved coronavirus antigens are: a conserved coronavirus NSP2 protein; a conserved coronavirus NSP3 protein; and a conserved coronavirus Nucleoprotein.

51. The composition of claim 31, wherein the at least two conserved coronavirus antigens are: a conserved coronavirus NSP2 protein; a conserved coronavirus NSP14 protein; and a conserved coronavirus Nucleoprotein.

52. The composition of claim 31, wherein the at least two conserved coronavirus antigens are: a conserved coronavirus NSP3 protein; a conserved coronavirus NSP14 protein; and a conserved coronavirus Nucleoprotein.

53.-57. (canceled)

58. The composition of claim 31 further comprising a T cell attracting chemokine, wherein the T cell attracting chemokine is CCL5, CXCL9, CXCL10, CXCL11, or a combination thereof.

59. The composition of claim 31 further comprising a composition that promotes T cell proliferation and T-cell memory, wherein the composition that promotes T cell proliferation and memory is IL-7, IL-2, or IL-15.

60. The composition of claim 31, wherein the conserved protein or antigen is conserved among human and animal coronaviruses.

61.-64. (canceled)

65. The composition of claim 31, wherein the composition comprises one of SEQ ID NO: 64-116.

66.-77. (canceled)

78. A pre-emptive pan-coronavirus vaccine composition, the composition comprising, or comprising a sequence encoding one or more large sequence coronavirus proteins, wherein the one or more large sequence coronavirus proteins comprise: a conserved coronavirus Spike protein; a conserved coronavirus NSP2 protein; a conserved coronavirus NSP14 protein; and a conserved coronavirus Nucleoprotein.

79. A method of preventing infection or reinfection by one or more coronavirus variants or subvariants in a subject, said method comprising administering a therapeutically effective amount of a composition according claim 31.


SEQUENCE LISTING (157 sequences)

SEQ ID NO: 1 - protein, 9 aa, synthetic construct
CLEASFNYL

SEQ ID NO: 2 - protein, 9 aa, synthetic construct
SLPGVFCGV

SEQ ID NO: 3 - protein, 9 aa, synthetic construct
ALNTLVKQL

SEQ ID NO: 4 - protein, 9 aa, synthetic construct
LLLDDFVEI

SEQ ID NO: 5 - protein, 9 aa, synthetic construct
ALNTLVKQL

SEQ ID NO: 6 - protein, 9 aa, synthetic construct
RLQSLQTYV

SEQ ID NO: 7 - protein, 9 aa, synthetic construct
FIAGLIAIV

SEQ ID NO: 8 - protein, 9 aa, synthetic construct
FLAFVVFLL

SEQ ID NO: 9 - protein, 9 aa, synthetic construct
FLLVTLAIL

SEQ ID NO: 10 - protein, 9 aa, synthetic construct
IFLWLLWPV

SEQ ID NO: 11 - protein, 9 aa, synthetic construct
GLMWLSYFI

SEQ ID NO: 12 - protein, 9 aa, synthetic construct
IHLVDFQVT

SEQ ID NO: 13 - protein, 9 aa, synthetic construct
IIFWFSLEL

SEQ ID NO: 14 - protein, 9 aa, synthetic construct
YIDIGNYTV

SEQ ID NO: 15 - protein, 9 aa, synthetic construct
YINVFAFPF

SEQ ID NO: 16 - protein, 9 aa, synthetic construct
NVFAFPFTI

SEQ ID NO: 17 - protein, 15 aa, synthetic construct
KSAFYILPSIISNEK

SEQ ID NO: 18 - protein, 13 aa, synthetic construct
MFVFLVLLPLVSS

SEQ ID NO: 19 - protein, 15 aa, synthetic construct
LSYYKLGASQRVAGD

SEQ ID NO: 20 - protein, 15 aa, synthetic construct
MKFLVFLGIITTVAA

SEQ ID NO: 21 - protein, 15 aa, synthetic construct
AEILLIIMRTFKVSI

SEQ ID NO: 22 - protein, 16 aa, synthetic construct
KQQTVTLLPAADLDDF

SEQ ID NO: 23 - protein, 25 aa, synthetic construct
SQCVNLTTRTQLPPAYTNSFTRGVY

SEQ ID NO: 24 - protein, 31 aa, synthetic construct
DAVDCALDPLSETKCTLKSFTVEKGIYQTSN

SEQ ID NO: 25 - protein, 26 aa, synthetic construct
FGEVFNATRFASVYAWNRKRISNCVA

SEQ ID NO: 26 - protein, 25 aa, synthetic construct
YNSASFSTFKCYGVSPTKLNDLCFT

SEQ ID NO: 27 - protein, 31 aa, synthetic construct
EIYQAGSTPCNGVEGFNCYFPLQSYGFQPTN

SEQ ID NO: 28 - protein, 35 aa, synthetic construct
VCGPKKSTNLVKNKCVNFNFNGLTGTGVLTESNKK

SEQ ID NO: 29 - protein, 34 aa, synthetic construct
FGRDIADTTDAVRDPQTLEILDITPCSFGGVSVI

SEQ ID NO: 30 - protein, 28 aa, synthetic construct
GTNTSNQVAVLYQDVNCTEVPVAIHADQ

SEQ ID NO: 31 - protein, 27 aa, synthetic construct
DVNCTEVPVAIHADQLTPTWRVYSTGS

SEQ ID NO: 32 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 33 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 34 - DNA, 635 bp, Homo sapiens
  source 1..635: mol_type = other DNA
gtatagggctgtctgggagccactccagggccacagaaatcttgtctctgactcagggtattttgttttctgttttgtgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgcc

SEQ ID NO: 35 - DNA, 1559 bp, Homo sapiens
  source 1..1559: mol_type = genomic DNA
catccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcgcccaagatgggagggtcctcagcttccccatctgtagaatgggcatcgtcccactcccatgacagagaggctcc

SEQ ID NO: 36 - DNA, 3665 bp, synthetic construct
  source 1..3665: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgttcgtgtttctggtcctgctgcctctggtgagctccctgctgctggacgactttgtggaaattgctgcttattacctcgcttccggcggccagccaatcgccgcctatgccctgaacaccctggtgaagcagctggctgcatattttctggctttcgtggtgtttctgctggccgcctacttcatcgccgggctgatcgctatcgtggccgcctattttctgctggtgacactggccattctggccgcctactgcctggaggcctccttcaactacctggccgcctactacatcgacatcggcaactacaccgtggccgcctatatcattttctggttctcactggagctggccgcttactccctgcccggcgtgttctgtggagtggccgcttacaacgtgtttgccttccccttcaccatcgcagcctacggtctgatgtggctgtcatacttcatcgcagcctatcatctggtggacttccaggtgactatcgcagcctacatcttcctgtggctgctgtggcccgtggccgcatactacattaatgtgttcgcctttccttttgccgcctacagactgcagagcctgcagacctacgtcggccctggccctgggaagtccgccttctacatcctgcccagtatcatttccaacgagaaggggccaggaccgggaatgttcgtgttcctggtgctgttgccactggtgagtagcggccccggaccaggcctgagctactataagctgggcgcctcccagcgggtggctggcgacggccccggaccaggcgctgagatcctgctgattatcatgaggaccttcaaggtgtccatcgggcctggtcctggaatgaagttcctggtgttcctgggcatcatcactaccgtggccgccgggcctgggccaggcaagcagcagaccgtgaccctgctgccagctgccgatctggatgatttcaagaaggacgccgtggactgcgccctggatcccctgtccgaaactaagtgtaccctgaagtctttcacagtggagaagggcatctaccagacaagcaacaagaagggcaccaatacttctaaccaggtcgccgtgctgtaccaggacgtgaactgcactgaagtccccgtggccattcacgctgaccaggacgtgaactgcacagaggtgccagtggccatccacgccgaccaactgacccccacctggcgagtgtactccactggctccaagaaatcccagtgcgtgaatttgactaccagaacacagctgccccccgcttacaccaacagtttcacaagaggggtctacaagaaggtgtgcggaccaaagaaaagcaccaacctggtgaaaaacaagtgcgtgaatttcaacttcaatggactgaccggcacgggagtgctgaccgagagcaataagaagaaaaagtttggacgcgacatcgccgatacaaccgatgccgtgagggacccacagacactggagatcctcgacatcaccccatgtagttttggcggagtgtcagtgatcaagaaggagatttaccaggccggcagcaccccatgcaacggcgtggaaggctttaattgctactttccactgcagtcctatggattccagcccaccaacaaaaagtataattctgcctcctttagcacttttaagtgctatggcgtgagccccacaaaactgaacgatctgtgcttcacaaagaagtttggcgaggtgttt

SEQ ID NO: 37 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - DNA, 3771 bp, synthetic construct
  source 1..3771: mol_type = other DNA
aacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 40 - DNA, 3771 bp, synthetic construct
  source 1..3771: mol_type = other DNA
aacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 41 - DNA, 3762 bp, synthetic construct
  source 1..3762: mol_type = other DNA
aacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 42 - DNA, 3762 bp, synthetic construct
  source 1..3762: mol_type = other DNA
aacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 43 - DNA, 2099 bp, synthetic construct
  source 1..2099: mol_type = other DNA
taccaagctactagagtagtggtactttcatttgagcttctaaatgcacctgccacagtgtgtggaccaaaattgtccacatcactaattaagaaccagtgtgtcaattttaatttcaatggactcaagggtactggtgtgttgactgactcgtccaaaaagtttcagtcttttcaacaatttggaagggatgcatctgattttactgactcagtacgcgaccctcagacacttcaaatacttgacatttcaccatgttcatttggtggtgtgagtgtaataacaccaggaacaaatgcttcatctgaagtagccgttctataccaagatgtaaactgcactgatgttcccacggccatacgtgctgaccaactcacacctgcttggcgtgtttactctgctggagtaaatgtgtttcaaactcaggctggctgtttaataggagcggaacatgtcaatgcttcatatgagtgtgacattcccattggtgcaggcatttgtgctagttaccatacagcttcccttttacgtaatacaggccagaaatcaattgtggcctatactatgtcacttggtgctgaaaactcaattgcttatgctaataactcaattgccatacctacaaatttttcaatcagtgtcacaactgaagtgatgcctgtttcaatggctaagacatcagtagattgtacaatgtacatctgtggtgactctcaggagtgcagcaacttactacttcagtatggtagcttttgcacacaattaaatcgtgccctttcaggcattgctgttgaacaggacaaaaacactcaagaggtttttgcccaagttaaacaaatgtataagacaccagccataaaagattttggtggctttaatttctcacaaatattgcctgacccttctaagccaacaaaaagatcatttattgaggatttactcttcaacaaagtgactctcgctgatgctggctttatgaagcaatacggcgaatgcctaggcgatattagtgctagagatctcatttgtgcgcagaagttcaatggactcactgtccttccacctctactcacggatgaaatgattgctgcttacaccgccgctcttgtcagcggtactgctactgctggttggacatttggtgcaggtgctgctctacaaataccttttgctatgcaaatggcttataggttcaatggcattggagttactcaaaatgttctctatgagaaccagaagcagatcgctaaccaatttaacaaggcgatcagtcaaattcaagaatcacttactactacttcaactgcattgggcaagctgcaagacgtcgtcaaccagaatgctcaagcattgaacacacttgttaaacaactaagttctaactttggtgcaatttcaagtgttttaaatgacattctgtctcgactygacaaagttgaggctgaagtgcaaattgataggttgattactggcagattacaaagccttcagacctatgtaacacaacaactaatcagagctgctgaaatcagagcttctgccaatcttgctgccactaagatgtccgagtgtgttcttggacaatcaaaaagagttgacttttgtggaaaaggctatcatcttatgtctttccctcaagcagccccacatggtgtcgtcttcttacatgtcacatacgtgccatcgcaagaaagaaacttcaccactgccccagcaatctgccatcaaggcaaggcacacttccctcgtgaaggtgtttttgtatctaatggcacttcttggtttatcacacagaggaacttcttttcaccacaaataattacaacagacaatacatttgtctctggaaattgtgatgtcgttattggcatcatcaacaatactgtttatgatcctctgcaacctgagcttgactcatttaaagaagagctggacaagtacttcaaaaaccacacgtcacctgatgtrgatcttggcgacatctcaggcattaatgcttcagtcgtcaatattcaaaaagaaattgaccgcctcaatgaggttgccaaaaatctaaatgaatcgctcatcgatcttcaagaacttggaaaatatgagca

SEQ ID NO: 44 - DNA, 540 bp, synthetic construct
  source 1..540: mol_type = other DNA
atggagagcctggtgcccggcttcaacgagaagacccacgtgcagctgagcctgcccgtgctgcaggtgagggacgtgctggtgaggggcttcggcgacagcgtggaggaggtgctgagcgaggccaggcagcacctgaaggacggcacctgcggcctggtggaggtggagaagggcgtgctgccccagctggagcagccctacgtgttcatcaagaggagcgacgccaggaccgccccccacggccacgtgatggtggagctggtggccgagctggagggcatccagtacggcaggagcggcgagaccctgggcgtgctggtgccccacgtgggcgagatccccgtggcctacaggaaggtgctgctgaggaagaacggcaacaagggcgccggcggccacagctacggcgccgacctgaagagcttcgacctgggcgacgagctgggcaccgacccctacgaggacttccaggagaactggaacaccaagcacagcagcggcgtgaccagggagctgatgagggagctgaacggcggc

SEQ ID NO: 45 - DNA, 1914 bp, synthetic construct
  source 1..1914: mol_type = other DNA
gcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggc

SEQ ID NO: 46 - DNA, 2289 bp, synthetic construct
  source 1..2289: mol_type = other DNA
atggcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcac

SEQ ID NO: 47 - DNA, 918 bp, synthetic construct
  source 1..918: mol_type = other DNA
agcggcttcaggaagatggccttccccagcggcaaggtggagggctgcatggtgcaggtgacctgcggcaccaccaccctgaacggcctgtggctggacgacgtggtgtactgccccaggcacgtgatctgcaccagcgaggacatgctgaaccccaactacgaggacctgctgatcaggaagagcaaccacaacttcctggtgcaggccggcaacgtgcagctgagggtgatcggccacagcatgcagaactgcgtgctgaagctgaaggtggacaccgccaaccccaagacccccaagtacaagttcgtgaggatccagcccggccagaccttcagcgtgctggcctgctacaacggcagccccagcggcgtgtaccagtgcgccatgaggcccaacttcaccatcaagggcagcttcctgaacggcagctgcggcagcgtgggcttcaacatcgactacgactgcgtgagcttctgctacatgcaccacatggagctgcccaccggcgtgcacgccggcaccgacctggagggcaacttctacggccccttcgtggacaggcagaccgcccaggccgccggcaccgacaccaccatcaccgtgaacgtgctggcctggctgtacgccgccgtgatcaacggcgacaggtggttcctgaacaggttcaccaccaccctgaacgacttcaacctggtggccatgaagtacaactacgagcccctgacccaggaccacgtggacatcctgggccccctgagcgcccagaccggcatcgccgtgctggacatgtgcgccagcctgaaggagctgctgcagaacggcatgaacggcaggaccatcctgggcagcgccctgctggaggacgagttcacccccttcgacgtggtgaggcagtgcagcggcgtgaccttccag

SEQ ID NO: 48 - DNA, 870 bp, synthetic construct
  source 1..870: mol_type = other DNA
agcgccgtgaagaggaccatcaagggcacccaccactggctgctgctgaccatcctgaccagcctgctggtgctggtgcagagcacccagtggagcctgttcttcttcctgtacgagaacgccttcctgcccttcgccatgggcatcatcgccatgagcgccttcgccatgatgttcgtgaagcacaagcacgccttcctgtgcctgttcctgctgcccagcctggccaccgtggcctacttcaacatggtgtacatgcccgccagctgggtgatgaggatcatgacctggctggacatggtggacaccagcctgagcggcttcaagctgaaggactgcgtgatgtacgccagcgccgtggtgctgctgatcctgatgaccgccaggaccgtgtacgacgacggcgccaggagggtgtggaccctgatgaacgtgctgaccctggtgtacaaggtgtactacggcaacgccctggaccaggccatcagcatgtgggccctgatcatcagcgtgaccagcaactacagcggcgtggtgaccaccgtgatgttcctggccaggggcatcgtgttcatgtgcgtggagtactgccccatcttcttcatcaccggcaacaccctgcagtgcatcatgctggtgtactgcttcctgggctacttctgcacctgctacttcggcctgttctgcctgctgaacaggtacttcaggctgaccctgggcgtgtacgactacctggtgagcacccaggagttcaggtacatgaacagccagggcctgctgccccccaagaacagcatcgacgccttcaagctgaacatcaagctgctgggcgtgggcggcaagccctgcatcaaggtggccaccgtgcag

SEQ ID NO: 49 - DNA, 249 bp, synthetic construct
  source 1..249: mol_type = other DNA
agcaagatgagcgacgtgaagtgcaccagcgtggtgctgctgagcgtgctgcagcagctgagggtggagagcagcagcaagctgtgggcccagtgcgtgcagctgcacaacgacatcctgctggccaaggacaccaccgaggccttcgagaagatggtgagcctgctgagcgtgctgctgagcatgcagggcgccgtggacatcaacaagctgtgcgaggagatgctggacaacagggccaccctgcag

SEQ ID NO: 50 - DNA, 593 bp, synthetic construct
  source 1..593: mol_type = other DNA
gccatcgccagcgagttcagcagcctgcccagctacgccgccttcgccaccgcccaggaggcctacgagcaggccgtggccaacggcgacagcgaggtggtgctgaagaagctgaagaagagcctgaacgtggccaagagcgagttcgacagggacgccgccatgcagaggaagctggagaagatggccgaccaggccatgacccagatgtacaagcaggccaggagcgaggacaagagggccaaggtgaccagcgccatgcagaccatgctgttcaccatgctgaggaagctggacaacgacgccctgaacaacatcatcaacaacgccagggacggctgcgtgcccctgaacatcatccccctgaccaccgccgccaagctgatggtggtgatccccgactacaacacctacaagaacacctgcgacggcaccaccttcacctacgccagcgccctgtgggagatccagcaggtggtggacgccgacagcaagatcgtgcagctgagcgagatcagcatggacaacagccccaacctggcctggcccctgatcgtgaccgccctgagggccaacagcgccgtgaagctgca

SEQ ID NO: 51 - DNA, 339 bp, synthetic construct
  source 1..339: mol_type = other DNA
aacaacgagctgagccccgtggccctgaggcagatgagctgcgccgccggcaccacccagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacctgtacttcatcaagggcctgaacaacctgaacaggggcatggtgctgggcagcctggccgccaccgtgaggctgcag

SEQ ID NO: 52 - DNA, 417 bp, synthetic construct
  source 1..417: mol_type = other DNA
gccggcaacgccaccgaggtgcccgccaacagcaccgtgctgagcttctgcgccttcgccgtggacgccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaagaacaccgtgtgcaccgtgtgcggcatgtggaagggctacggctgcagctgcgaccagctgagggagcccatgctgcag

SEQ ID NO: 53 - DNA, 2799 bp, synthetic construct
  source 1..2799: mol_type = other DNA
atgagcgccgacgcccagagcttcctgaacagggtgtgcggcgtgagcgccgccaggctgaccccctgcggcaccggcaccagcaccgacgtggtgtacagggccttcgacatctacaacgacaaggtggccggcttcgccaagttcctgaagaccaactgctgcaggttccaggagaaggacgaggacgacaacctgatcgacagctacttcgtggtgaagaggcacaccttcagcaactaccagcacgaggagaccatctacaacctgctgaaggactgccccgccgtggccaagcacgacttcttcaagttcaggatcgacggcgacatggtgccccacatcagcaggcagaggctgaccaagtacaccatggccgacctggtgtacgccctgaggcacttcgacgagggcaactgcgacaccctgaaggagatcctggtgacctacaactgctgcgacgacgactacttcaacaagaaggactggtacgacttcgtggagaaccccgacatcctgagggtgtacgccaacctgggcgagagggtgaggcaggccctgctgaagaccgtgcagttctgcgacgccatgaggaacgccggcatcgtgggcgtgctgaccctggacaaccaggacctgaacggcaactggtacgacttcggcgacttcatccagaccacccccggcagcggcgtgcccgtggtggacagctactacagcctgctgatgcccatcctgaccctgaccagggccctgaccgccgagagccacgtggacaccgacctgaccaagccctacatcaagtgggacctgctgaagtacgacttcaccgaggagaggctgaagctgttcgacaggtacttcaagtactgggaccagacctaccaccccaactgcgtgaactgcctggacgacaggtgcatcctgcactgcgccaacttcaacgtgctgttcagcaccgtgttcccccccaccagcttcggccccctggtgaggaagatcttcgtggacggcgtgcccttcgtggtgagcaccggctaccacttcagggagctgggcgtggtgcacaaccaggacgtgaacctgcacagcagcaggctgagcttcaaggagctgctggtgtacgccgccgaccccgccatgcacgccgccagcggcaacctgctgctggacaagaggaccacctgcttcagcgtggccgccctgaccaacaacgtggccttccagaccgtgaagcccggcaacttcaacaaggacttctacgacttcgccgtgagcaagggcttcttcaaggagggcagcagcgtggagctgaagcacttcttcttcgcccaggacggcaacgccgccatcagcgactacgactactacaggtacaacctgcccaccatgtgcgacatcaggcagctgctgttcgtggtggaggtggtggacaagtacttcgactgctacgacggcggctgcatcaacgccaaccaggtgatcgtgaacaacctggacaagagcgccggcttccccttcaacaagtggggcaaggccaggctgtactacgacagcatgagctacgaggaccaggacgccctgttcgcctacaccaagaggaacgtgatccccaccatcacccagatgaacctgaagtacgccatcagcgccaagaacagggccaggaccgtggccggcgtgagcatctgcagcaccatgaccaacaggcagttccaccagaagctgctgaagagcatcgccgccaccaggggcgccaccgtggtgatcggcaccagcaagttctacggcggctggcacaacatgctgaagaccgtgtacagcgacgtggagaacccccacctgatgggctgggactaccccaagtgcgacagggccatgcccaacatgctgaggatcatggccagcctggtgctggccaggaagcacaccacctgctgcagcctgagccacaggttctacaggctggccaacgagtgcgcccaggtgctgagcgagatggtgatgtgcggcggcagcctgtacgtgaagcccggcggcaccagcagcggcgacgccaccaccgcctacgccaacagcgtgttcaacatctgccaggccgtgaccgccaacgtgaacgccctgctgagcaccgacggcaacaagatcgccgacaagtacgtgaggaacctgcagcacaggctgtacgagtgcctgtacaggaacagggacgtggacaccgacttcgtgaacgagttctacgcctacctgaggaagcacttcagcatgatgatcctgagcgacgacgccgtggtgtgcttcaacagcacctacgccagccagggcctggtggccagcatcaagaacttcaagagcgtgctgtactaccagaacaacgtgttcatgagcgaggccaagtgctggaccgagaccgacctgaccaagggcccccacgagttctgcagccagcacaccatgctggtgaagcagggcgacgactacgtgtacctgccctaccccgaccccagcaggatcctgggcgccggctgcttcgtggacgacatcgtgaagaccgacggcaccctgatgatcgagaggttcgtgagcctggccatcgacgcctaccccctgaccaagcaccccaaccaggagtacgccgacgtgttccacctgtacctgcagtacatcaggaagctgcacgacgagctgaccggccacatgctggacatgtacagcgtgatgctgaccaacgacaacaccagcaggtactgggagcccgagttctacgaggccatgtacaccccccacaccgtgctgcag

SEQ ID NO: 54 - DNA, 1803 bp, synthetic construct
  source 1..1803: mol_type = other DNA
gccgtgggcgcctgcgtgctgtgcaacagccagaccagcctgaggtgcggcgcctgcatcaggaggcccttcctgtgctgcaagtgctgctacgaccacgtgatcagcaccagccacaagctggtgctgagcgtgaacccctacgtgtgcaacgcccccggctgcgacgtgaccgacgtgacccagctgtacctgggcggcatgagctactactgcaagagccacaagccccccatcagcttccccctgtgcgccaacggccaggtgttcggcctgtacaagaacacctgcgtgggcagcgacaacgtgaccgacttcaacgccatcgccacctgcgactggaccaacgccggcgactacatcctggccaacacctgcaccgagaggctgaagctgttcgccgccgagaccctgaaggccaccgaggagaccttcaagctgagctacggcatcgccaccgtgagggaggtgctgagcgacagggagctgcacctgagctgggaggtgggcaagcccaggccccccctgaacaggaactacgtgttcaccggctacagggtgaccaagaacagcaaggtgcagatcggcgagtacaccttcgagaagggcgactacggcgacgccgtggtgtacaggggcaccaccacctacaagctgaacgtgggcgactacttcgtgctgaccagccacaccgtgatgcccctgagcgcccccaccctggtgccccaggagcactacgtgaggatcaccggcctgtaccccaccctgaacatcagcgacgagttcagcagcaacgtggccaactaccagaaggtgggcatgcagaagtacagcaccctgcagggcccccccggcaccggcaagagccacttcgccatcggcctggccctgtactaccccagcgccaggatcgtgtacaccgcctgcagccacgccgccgtggacgccctgtgcgagaaggccctgaagtacctgcccatcgacaagtgcagcaggatcatccccgccagggccagggtggagtgcttcgacaagttcaaggtgaacagcaccctggagcagtacgtgttctgcaccgtgaacgccctgcccgagaccaccgccgacatcgtggtgttcgacgagatcagcatggccaccaactacgacctgagcgtggtgaacgccaggctgagggccaagcactacgtgtacatcggcgaccccgcccagctgcccgcccccaggaccctgctgaccaagggcaccctggagcccgagtacttcaacagcgtgtgcaggctgatgaagaccatcggccccgacatgttcctgggcacctgcaggaggtgccccgccgagatcgtggacaccgtgagcgccctggtgtacgacaacaagctgaaggcccacaaggacaagagcgcccagtgcttcaagatgttctacaagggcgtgatcacccacgacgtgagcagcgccatcaacaggccccagatcggcgtggtgagggagttcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcag

SEQ ID NO: 55 - DNA, 1581 bp, synthetic construct
  source 1..1581: mol_type = other DNA
gccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcag

SEQ ID NO: 56 - DNA, 1038 bp, synthetic construct
  source 1..1038: mol_type = other DNA
agcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcag

SEQ ID NO: 57 - DNA, 894 bp, synthetic construct
  source 1..894: mol_type = other DNA
agcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagctgaggggcaccgccgtgatgagcctgaaggagggccagatcaacgacatgatcctgagcctgctgagcaagggcaggctgatcatcagggagaacaacagggtggtgatcagcagcgacgtgctggtgaacaac

SEQ ID NO: 58 - DNA, 825 bp, synthetic construct
  source 1..825: mol_type = other DNA
atggacctgttcatgaggatcttcaccatcggcaccgtgaccctgaagcagggcgagatcaaggacgccacccccagcgacttcgtgagggccaccgccaccatccccatccaggccagcctgcccttcggctggctgatcgtgggcgtggccctgctggccgtgttccagagcgccagcaagatcatcaccctgaagaagaggtggcagctggccctgagcaagggcgtgcacttcgtgtgcaacctgctgctgctgttcgtgaccgtgtacagccacctgctgctggtggccgccggcctggaggcccccttcctgtacctgtacgccctggtgtacttcctgcagagcatcaacttcgtgaggatcatcatgaggctgtggctgtgctggaagtgcaggagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctg

SEQ ID NO: 59 - DNA, 1254 bp, synthetic construct
  source 1..1254: mol_type = other DNA
agcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcc

SEQ ID NO: 60 - DNA, 3684 bp, synthetic construct
  source 1..3684: mol_type = other DNA
gaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcaggccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagct

SEQ ID NO: 61 - DNA, 225 bp, synthetic construct
  source 1..225: mol_type = other DNA
atgtacagcttcgtgagcgaggagaccggcaccctgatcgtgaacagcgtgctgctgttcctggccttcgtggtgttcctgctggtgaccctggccatcctgaccgccctgaggctgtgcgcctactgctgcaacatcgtgaacgtgagcctggtgaagcccagcttctacgtgtacagcagggtgaagaacctgaacagcagcagggtgcccgacctgctggtg

SEQ ID NO: 62 - DNA, 663 bp, synthetic construct
  source 1..663: mol_type = other DNA
gccgacagcaacggcaccatcaccgtggaggagctgaagaagctgctggagcagtggaacctggtgatcggcttcctgttcctgacctggatctgcctgctgcagttcgcctacgccaacaggaacaggttcctgtacatcatcaagctgatcttcctgtggctgctgtggcccgtgaccctggcctgcttcgtgctggccgccgtgtacaggatcaactggatcaccggcggcatcgccatcgccatggcctgcctggtgggcctgatgtggctgagctacttcatcgccagcttcaggctgttcgccaggaccaggagcatgtggagcttcaaccccgagaccaacatcctgctgaacgtgcccctgcacggcaccatcctgaccaggcccctgctggagagcgagctggtgatcggcgccgtgatcctgaggggccacctgaggatcgccggccaccacctgggcaggtgcgacatcaaggacctgcccaaggagatcaccgtggccaccagcaggaccctgagctactacaagctgggcgccagccagagggtggccggcgacagcggcttcgccgcctacagcaggtacaggatcggcaactacaagctgaacaccgaccacagcagcagcagcgacaacatcgccctgctggtgcag

SEQ ID NO: 63 - DNA, 84 bp, synthetic construct
  source 1..84: mol_type = other DNA
aagatcatcctgttcctggccctgatcaccctggccacctgcgagctgtaccactaccaggagtgcgtgaggggcaccaccgtg

SEQ ID NO: 64 - DNA, 7354 bp, synthetic construct
  source 1..7354: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggagagcctggtgcccggcttcaacgagaagacccacgtgcagctgagcctgcccgtgctgcaggtgagggacgtgctggtgaggggcttcggcgacagcgtggaggaggtgctgagcgaggccaggcagcacctgaaggacggcacctgcggcctggtggaggtggagaagggcgtgctgccccagctggagcagccctacgtgttcatcaagaggagcgacgccaggaccgccccccacggccacgtgatggtggagctggtggccgagctggagggcatccagtacggcaggagcggcgagaccctgggcgtgctggtgccccacgtgggcgagatccccgtggcctacaggaaggtgctgctgaggaagaacggcaacaagggcgccggcggccacagctacggcgccgacctgaagagcttcgacctgggcgacgagctgggcaccgacccctacgaggacttccaggagaactggaacaccaagcacagcagcggcgtgaccagggagctgatgagggagctgaacggcggcggaagcggagagggcaggggaagtcttctaacatgcggggacgtggaggaaaatcccggccccgcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 65 - DNA, 7354 bp, synthetic construct
  source 1..7354: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggagagcctggtgcccggcttcaacgagaagacccacgtgcagctgagcctgcccgtgctgcaggtgagggacgtgctggtgaggggcttcggcgacagcgtggaggaggtgctgagcgaggccaggcagcacctgaaggacggcacctgcggcctggtggaggtggagaagggcgtgctgccccagctggagcagccctacgtgttcatcaagaggagcgacgccaggaccgccccccacggccacgtgatggtggagctggtggccgagctggagggcatccagtacggcaggagcggcgagaccctgggcgtgctggtgccccacgtgggcgagatccccgtggcctacaggaaggtgctgctgaggaagaacggcaacaagggcgccggcggccacagctacggcgccgacctgaagagcttcgacctgggcgacgagctgggcaccgacccctacgaggacttccaggagaactggaacaccaagcacagcagcggcgtgaccagggagctgatgagggagctgaacggcggcggaagcggagagggcaggggaagtcttctaacatgcggggacgtggaggaaaatcccggccccgcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 66 - DNA, 6682 bp, synthetic construct
  source 1..6682: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcgccgtgaagaggaccatcaagggcacccaccactggctgctgctgaccatcctgaccagcctgctggtgctggtgcagagcacccagtggagcctgttcttcttcctgtacgagaacgccttcctgcccttcgccatgggcatcatcgccatgagcgccttcgccatgatgttcgtgaagcacaagcacgccttcctgtgcctgttcctgctgcccagcctggccaccgtggcctacttcaacatggtgtacatgcccgccagctgggtgatgaggatcatgacctggctggacatggtggacaccagcctgagcggcttcaagctgaaggactgcgtgatgtacgccagcgccgtggtgctgctgatcctgatgaccgccaggaccgtgtacgacgacggcgccaggagggtgtggaccctgatgaacgtgctgaccctggtgtacaaggtgtactacggcaacgccctggaccaggccatcagcatgtgggccctgatcatcagcgtgaccagcaactacagcggcgtggtgaccaccgtgatgttcctggccaggggcatcgtgttcatgtgcgtggagtactgccccatcttcttcatcaccggcaacaccctgcagtgcatcatgctggtgtactgcttcctgggctacttctgcacctgctacttcggcctgttctgcctgctgaacaggtacttcaggctgaccctgggcgtgtacgactacctggtgagcacccaggagttcaggtacatgaacagccagggcctgctgccccccaagaacagcatcgacgccttcaagctgaacatcaagctgctgggcgtgggcggcaagccctgcatcaaggtggccaccgtgcagggaagcggagagggcaggggaagtcttctaacatgcggggacgtggaggaaaatcccggccccagcaagatgagcgacgtgaagtgcaccagcgtggtgctgctgagcgtgctgcagcagctgagggtggagagcagcagcaagctgtgggcccagtgcgtgcagctgcacaacgacatcctgctggccaaggacaccaccgaggccttcgagaagatggtgagcctgctgagcgtgctgctgagcatgcagggcgccgtggacatcaacaagctgtgcgaggagatgctggacaacagggccaccctgcagggaagcggacagtgtactaattatgctctcttgaaattggctggagatgttgagagcaacccaggtcccgccatcgccagcgagttcagcagcctgcccagctacgccgccttcgccaccgcccaggaggcctacgagcaggccgtggccaacggcgacagcgaggtggtgctgaagaagctgaagaagagcctgaacgtggccaagagcgagttcgacagggacgccgccatgcagaggaagctggagaagatggccgaccaggccatgacccagatgtacaagcaggccaggagcgaggacaagagggccaaggtgaccagcgccatgcagaccatgctgttcaccatgctgaggaagctggacaacgacgccctgaacaacatcatcaacaacgccagggacggctgcgtgcccctgaacatcatccccctgaccaccgccgccaagctgatggtggtgatccccgactacaacacctacaagaacacctgcgacggcaccaccttcacctacgccagcgccctgtgggagatccagcaggtggtggacgccgacagcaagatcgtgcagctgagcgagatcagcatggacaacagccccaacctggcctggcccctgatcgtgaccgccctgagggccaacagcgccgtgaagctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 67 - DNA, 6682 bp, synthetic construct
  source 1..6682: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcgccgtgaagaggaccatcaagggcacccaccactggctgctgctgaccatcctgaccagcctgctggtgctggtgcagagcacccagtggagcctgttcttcttcctgtacgagaacgccttcctgcccttcgccatgggcatcatcgccatgagcgccttcgccatgatgttcgtgaagcacaagcacgccttcctgtgcctgttcctgctgcccagcctggccaccgtggcctacttcaacatggtgtacatgcccgccagctgggtgatgaggatcatgacctggctggacatggtggacaccagcctgagcggcttcaagctgaaggactgcgtgatgtacgccagcgccgtggtgctgctgatcctgatgaccgccaggaccgtgtacgacgacggcgccaggagggtgtggaccctgatgaacgtgctgaccctggtgtacaaggtgtactacggcaacgccctggaccaggccatcagcatgtgggccctgatcatcagcgtgaccagcaactacagcggcgtggtgaccaccgtgatgttcctggccaggggcatcgtgttcatgtgcgtggagtactgccccatcttcttcatcaccggcaacaccctgcagtgcatcatgctggtgtactgcttcctgggctacttctgcacctgctacttcggcctgttctgcctgctgaacaggtacttcaggctgaccctgggcgtgtacgactacctggtgagcacccaggagttcaggtacatgaacagccagggcctgctgccccccaagaacagcatcgacgccttcaagctgaacatcaagctgctgggcgtgggcggcaagccctgcatcaaggtggccaccgtgcagggaagcggagagggcaggggaagtcttctaacatgcggggacgtggaggaaaatcccggccccagcaagatgagcgacgtgaagtgcaccagcgtggtgctgctgagcgtgctgcagcagctgagggtggagagcagcagcaagctgtgggcccagtgcgtgcagctgcacaacgacatcctgctggccaaggacaccaccgaggccttcgagaagatggtgagcctgctgagcgtgctgctgagcatgcagggcgccgtggacatcaacaagctgtgcgaggagatgctggacaacagggccaccctgcagggaagcggacagtgtactaattatgctctcttgaaattggctggagatgttgagagcaacccaggtcccgccatcgccagcgagttcagcagcctgcccagctacgccgccttcgccaccgcccaggaggcctacgagcaggccgtggccaacggcgacagcgaggtggtgctgaagaagctgaagaagagcctgaacgtggccaagagcgagttcgacagggacgccgccatgcagaggaagctggagaagatggccgaccaggccatgacccagatgtacaagcaggccaggagcgaggacaagagggccaaggtgaccagcgccatgcagaccatgctgttcaccatgctgaggaagctggacaacgacgccctgaacaacatcatcaacaacgccagggacggctgcgtgcccctgaacatcatccccctgaccaccgccgccaagctgatggtggtgatccccgactacaacacctacaagaacacctgcgacggcaccaccttcacctacgccagcgccctgtgggagatccagcaggtggtggacgccgacagcaagatcgtgcagctgagcgagatcagcatggacaacagccccaacctggcctggcccctgatcgtgaccgccctgagggccaacagcgccgtgaagctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 68 - DNA, 7528 bp, synthetic construct
  source 1..7528: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctaacaacgagctgagccccgtggccctgaggcagatgagctgcgccgccggcaccacccagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacctgtacttcatcaagggcctgaacaacctgaacaggggcatggtgctgggcagcctggccgccaccgtgaggctgcagggaagcggagagggcaggggaagtcttctaacatgcggggacgtggaggaaaatcccggccccgccggcaacgccaccgaggtgcccgccaacagcaccgtgctgagcttctgcgccttcgccgtggacgccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaagaacaccgtgtgcaccgtgtgcggcatgtggaagggctacggctgcagctgcgaccagctgagggagcccatgctgcagggaagcggacagtgtactaattatgctctcttgaaattggctggagatgttgagagcaacccaggtcccgccgtgggcgcctgcgtgctgtgcaacagccagaccagcctgaggtgcggcgcctgcatcaggaggcccttcctgtgctgcaagtgctgctacgaccacgtgatcagcaccagccacaagctggtgctgagcgtgaacccctacgtgtgcaacgcccccggctgcgacgtgaccgacgtgacccagctgtacctgggcggcatgagctactactgcaagagccacaagccccccatcagcttccccctgtgcgccaacggccaggtgttcggcctgtacaagaacacctgcgtgggcagcgacaacgtgaccgacttcaacgccatcgccacctgcgactggaccaacgccggcgactacatcctggccaacacctgcaccgagaggctgaagctgttcgccgccgagaccctgaaggccaccgaggagaccttcaagctgagctacggcatcgccaccgtgagggaggtgctgagcgacagggagctgcacctgagctgggaggtgggcaagcccaggccccccctgaacaggaactacgtgttcaccggctacagggtgaccaagaacagcaaggtgcagatcggcgagtacaccttcgagaagggcgactacggcgacgccgtggtgtacaggggcaccaccacctacaagctgaacgtgggcgactacttcgtgctgaccagccacaccgtgatgcccctgagcgcccccaccctggtgccccaggagcactacgtgaggatcaccggcctgtaccccaccctgaacatcagcgacgagttcagcagcaacgtggccaactaccagaaggtgggcatgcagaagtacagcaccctgcagggcccccccggcaccggcaagagccacttcgccatcggcctggccctgtactaccccagcgccaggatcgtgtacaccgcctgcagccacgccgccgtggacgccctgtgcgagaaggccctgaagtacctgcccatcgacaagtgcagcaggatcatccccgccagggccagggtggagtgcttcgacaagttcaaggtgaacagcaccctggagcagtacgtgttctgcaccgtgaacgccctgcccgagaccaccgccgacatcgtggtgttcgacgagatcagcatggccaccaactacgacctgagcgtggtgaacgccaggctgagggccaagcactacgtgtacatcggcgaccccgcccagctgcccgcccccaggaccctgctgaccaagggcaccctggagcccgagtacttcaacagcgtgtgcaggctgatgaagaccatcggccccgacatgttcctgggcacctgcaggaggtgccccgccgagatcgtggacaccgtgagcgccctggtgtacgacaacaagctgaaggcccacaaggacaagagcgcccagtgcttcaagatgttctacaagggcgtgatcacccacgacgtgagcagcgccatcaacaggccccagatcggcgtggtgagggagttcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 69 - DNA, 7528 bp, synthetic construct
  source 1..7528: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctaacaacgagctgagccccgtggccctgaggcagatgagctgcgccgccggcaccacccagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacctgtacttcatcaagggcctgaacaacctgaacaggggcatggtgctgggcagcctggccgccaccgtgaggctgcagggaagcggagagggcaggggaagtcttctaacatgcggggacgtggaggaaaatcccggccccgccggcaacgccaccgaggtgcccgccaacagcaccgtgctgagcttctgcgccttcgccgtggacgccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaagaacaccgtgtgcaccgtgtgcggcatgtggaagggctacggctgcagctgcgaccagctgagggagcccatgctgcagggaagcggacagtgtactaattatgctctcttgaaattggctggagatgttgagagcaacccaggtcccgccgtgggcgcctgcgtgctgtgcaacagccagaccagcctgaggtgcggcgcctgcatcaggaggcccttcctgtgctgcaagtgctgctacgaccacgtgatcagcaccagccacaagctggtgctgagcgtgaacccctacgtgtgcaacgcccccggctgcgacgtgaccgacgtgacccagctgtacctgggcggcatgagctactactgcaagagccacaagccccccatcagcttccccctgtgcgccaacggccaggtgttcggcctgtacaagaacacctgcgtgggcagcgacaacgtgaccgacttcaacgccatcgccacctgcgactggaccaacgccggcgactacatcctggccaacacctgcaccgagaggctgaagctgttcgccgccgagaccctgaaggccaccgaggagaccttcaagctgagctacggcatcgccaccgtgagggaggtgctgagcgacagggagctgcacctgagctgggaggtgggcaagcccaggccccccctgaacaggaactacgtgttcaccggctacagggtgaccaagaacagcaaggtgcagatcggcgagtacaccttcgagaagggcgactacggcgacgccgtggtgtacaggggcaccaccacctacaagctgaacgtgggcgactacttcgtgctgaccagccacaccgtgatgcccctgagcgcccccaccctggtgccccaggagcactacgtgaggatcaccggcctgtaccccaccctgaacatcagcgacgagttcagcagcaacgtggccaactaccagaaggtgggcatgcagaagtacagcaccctgcagggcccccccggcaccggcaagagccacttcgccatcggcctggccctgtactaccccagcgccaggatcgtgtacaccgcctgcagccacgccgccgtggacgccctgtgcgagaaggccctgaagtacctgcccatcgacaagtgcagcaggatcatccccgccagggccagggtggagtgcttcgacaagttcaaggtgaacagcaccctggagcagtacgtgttctgcaccgtgaacgccctgcccgagaccaccgccgacatcgtggtgttcgacgagatcagcatggccaccaactacgacctgagcgtggtgaacgccaggctgagggccaagcactacgtgtacatcggcgaccccgcccagctgcccgcccccaggaccctgctgaccaagggcaccctggagcccgagtacttcaacagcgtgtgcaggctgatgaagaccatcggccccgacatgttcctgggcacctgcaggaggtgccccgccgagatcgtggacaccgtgagcgccctggtgtacgacaacaagctgaaggcccacaaggacaagagcgcccagtgcttcaagatgttctacaagggcgtgatcacccacgacgtgagcagcgccatcaacaggccccagatcggcgtggtgagggagttcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 70 - DNA, 7519 bp, synthetic construct
  source 1..7519: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagggaagcggagagggcaggggaagtcttctaacatgcggggacgtggaggaaaatcccggccccagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 71 - DNA, 7519 bp, synthetic construct
  source 1..7519: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagggaagcggagagggcaggggaagtcttctaacatgcggggacgtggaggaaaatcccggccccagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 72 - DNA, 5731 bp, synthetic construct
  source 1..5731: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagctgaggggcaccgccgtgatgagcctgaaggagggccagatcaacgacatgatcctgagcctgctgagcaagggcaggctgatcatcagggagaacaacagggtggtgatcagcagcgacgtgctggtgaacaactgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 73 - DNA, 5731 bp, synthetic construct
  source 1..5731: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagctgaggggcaccgccgtgatgagcctgaaggagggccagatcaacgacatgatcctgagcctgctgagcaagggcaggctgatcatcagggagaacaacagggtggtgatcagcagcgacgtgctggtgaacaactgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 74 - DNA, 5662 bp, synthetic construct
  source 1..5662: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggacctgttcatgaggatcttcaccatcggcaccgtgaccctgaagcagggcgagatcaaggacgccacccccagcgacttcgtgagggccaccgccaccatccccatccaggccagcctgcccttcggctggctgatcgtgggcgtggccctgctggccgtgttccagagcgccagcaagatcatcaccctgaagaagaggtggcagctggccctgagcaagggcgtgcacttcgtgtgcaacctgctgctgctgttcgtgaccgtgtacagccacctgctgctggtggccgccggcctggaggcccccttcctgtacctgtacgccctggtgtacttcctgcagagcatcaacttcgtgaggatcatcatgaggctgtggctgtgctggaagtgcaggagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 75 - DNA, 5662 bp, synthetic construct
  source 1..5662: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggacctgttcatgaggatcttcaccatcggcaccgtgaccctgaagcagggcgagatcaaggacgccacccccagcgacttcgtgagggccaccgccaccatccccatccaggccagcctgcccttcggctggctgatcgtgggcgtggccctgctggccgtgttccagagcgccagcaagatcatcaccctgaagaagaggtggcagctggccctgagcaagggcgtgcacttcgtgtgcaacctgctgctgctgttcgtgaccgtgtacagccacctgctgctggtggccgccggcctggaggcccccttcctgtacctgtacgccctggtgtacttcctgcagagcatcaacttcgtgaggatcatcatgaggctgtggctgtgctggaagtgcaggagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 76 - DNA, 6408 bp, synthetic construct
  source 1..6408: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggagagcctggtgcccggcttcaacgagaagacccacgtgcagctgagcctgcccgtgctgcaggtgagggacgtgctggtgaggggcttcggcgacagcgtggaggaggtgctgagcgaggccaggcagcacctgaaggacggcacctgcggcctggtggaggtggagaagggcgtgctgccccagctggagcagccctacgtgttcatcaagaggagcgacgccaggaccgccccccacggccacgtgatggtggagctggtggccgagctggagggcatccagtacggcaggagcggcgagaccctgggcgtgctggtgccccacgtgggcgagatccccgtggcctacaggaaggtgctgctgaggaagaacggcaacaagggcgccggcggccacagctacggcgccgacctgaagagcttcgacctgggcgacgagctgggcaccgacccctacgaggacttccaggagaactggaacaccaagcacagcagcggcgtgaccagggagctgatgagggagctgaacggcggcggaagcggagagggcaggggaagtcttctaacatgcggggacgtggaggaaaatcccggccccgcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggctgatga

SEQ ID NO: 77 - DNA, 6408 bp, synthetic construct
  source 1..6408: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggagagcctggtgcccggcttcaacgagaagacccacgtgcagctgagcctgcccgtgctgcaggtgagggacgtgctggtgaggggcttcggcgacagcgtggaggaggtgctgagcgaggccaggcagcacctgaaggacggcacctgcggcctggtggaggtggagaagggcgtgctgccccagctggagcagccctacgtgttcatcaagaggagcgacgccaggaccgccccccacggccacgtgatggtggagctggtggccgagctggagggcatccagtacggcaggagcggcgagaccctgggcgtgctggtgccccacgtgggcgagatccccgtggcctacaggaaggtgctgctgaggaagaacggcaacaagggcgccggcggccacagctacggcgccgacctgaagagcttcgacctgggcgacgagctgggcaccgacccctacgaggacttccaggagaactggaacaccaagcacagcagcggcgtgaccagggagctgatgagggagctgaacggcggcggaagcggagagggcaggggaagtcttctaacatgcggggacgtggaggaaaatcccggccccgcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggctgatga

SEQ ID NO: 78 - DNA, 5736 bp, synthetic construct
  source 1..5736: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcgccgtgaagaggaccatcaagggcacccaccactggctgctgctgaccatcctgaccagcctgctggtgctggtgcagagcacccagtggagcctgttcttcttcctgtacgagaacgccttcctgcccttcgccatgggcatcatcgccatgagcgccttcgccatgatgttcgtgaagcacaagcacgccttcctgtgcctgttcctgctgcccagcctggccaccgtggcctacttcaacatggtgtacatgcccgccagctgggtgatgaggatcatgacctggctggacatggtggacaccagcctgagcggcttcaagctgaaggactgcgtgatgtacgccagcgccgtggtgctgctgatcctgatgaccgccaggaccgtgtacgacgacggcgccaggagggtgtggaccctgatgaacgtgctgaccctggtgtacaaggtgtactacggcaacgccctggaccaggccatcagcatgtgggccctgatcatcagcgtgaccagcaactacagcggcgtggtgaccaccgtgatgttcctggccaggggcatcgtgttcatgtgcgtggagtactgccccatcttcttcatcaccggcaacaccctgcagtgcatcatgctggtgtactgcttcctgggctacttctgcacctgctacttcggcctgttctgcctgctgaacaggtacttcaggctgaccctgggcgtgtacgactacctggtgagcacccaggagttcaggtacatgaacagccagggcctgctgccccccaagaacagcatcgacgccttcaagctgaacatcaagctgctgggcgtgggcggcaagccctgcatcaaggtggccaccgtgcagggaagcggagagggcaggggaagtcttctaacatgcggggacgtggaggaaaatcccggccccagcaagatgagcgacgtgaagtgcaccagcgtggtgctgctgagcgtgctgcagcagctgagggtggagagcagcagcaagctgtgggcccagtgcgtgcagctgcacaacgacatcctgctggccaaggacaccaccgaggccttcgagaagatggtgagcctgctgagcgtgctgctgagcatgcagggcgccgtggacatcaacaagctgtgcgaggagatgctggacaacagggccaccctgcagggaagcggacagtgtactaattatgctctcttgaaattggctggagatgttgagagcaacccaggtcccgccatcgccagcgagttcagcagcctgcccagctacgccgccttcgccaccgcccaggaggcctacgagcaggccgtggccaacggcgacagcgaggtggtgctgaagaagctgaagaagagcctgaacgtggccaagagcgagttcgacagggacgccgccatgcagaggaagctggagaagatggccgaccaggccatgacccagatgtacaagcaggccaggagcgaggacaagagggccaaggtgaccagcgccatgcagaccatgctgttcaccatgctgaggaagctggacaacgacgccctgaacaacatcatcaacaacgccagggacggctgcgtgcccctgaacatcatccccctgaccaccgccgccaagctgatggtggtgatccccgactacaacacctacaagaacacctgcgacggcaccaccttcacctacgccagcgccctgtgggagatccagcaggtggtggacgccgacagcaagatcgtgcagctgagcgagatcagcatggacaacagccccaacctggcctggcccctgatcgtgaccgccctgagggccaacagcgccgtgaagctgcagtgatga

SEQ ID NO: 79 - DNA, 5736 bp, synthetic construct
  source 1..5736: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcgccgtgaagaggaccatcaagggcacccaccactggctgctgctgaccatcctgaccagcctgctggtgctggtgcagagcacccagtggagcctgttcttcttcctgtacgagaacgccttcctgcccttcgccatgggcatcatcgccatgagcgccttcgccatgatgttcgtgaagcacaagcacgccttcctgtgcctgttcctgctgcccagcctggccaccgtggcctacttcaacatggtgtacatgcccgccagctgggtgatgaggatcatgacctggctggacatggtggacaccagcctgagcggcttcaagctgaaggactgcgtgatgtacgccagcgccgtggtgctgctgatcctgatgaccgccaggaccgtgtacgacgacggcgccaggagggtgtggaccctgatgaacgtgctgaccctggtgtacaaggtgtactacggcaacgccctggaccaggccatcagcatgtgggccctgatcatcagcgtgaccagcaactacagcggcgtggtgaccaccgtgatgttcctggccaggggcatcgtgttcatgtgcgtggagtactgccccatcttcttcatcaccggcaacaccctgcagtgcatcatgctggtgtactgcttcctgggctacttctgcacctgctacttcggcctgttctgcctgctgaacaggtacttcaggctgaccctgggcgtgtacgactacctggtgagcacccaggagttcaggtacatgaacagccagggcctgctgccccccaagaacagcatcgacgccttcaagctgaacatcaagctgctgggcgtgggcggcaagccctgcatcaaggtggccaccgtgcagggaagcggagagggcaggggaagtcttctaacatgcggggacgtggaggaaaatcccggccccagcaagatgagcgacgtgaagtgcaccagcgtggtgctgctgagcgtgctgcagcagctgagggtggagagcagcagcaagctgtgggcccagtgcgtgcagctgcacaacgacatcctgctggccaaggacaccaccgaggccttcgagaagatggtgagcctgctgagcgtgctgctgagcatgcagggcgccgtggacatcaacaagctgtgcgaggagatgctggacaacagggccaccctgcagggaagcggacagtgtactaattatgctctcttgaaattggctggagatgttgagagcaacccaggtcccgccatcgccagcgagttcagcagcctgcccagctacgccgccttcgccaccgcccaggaggcctacgagcaggccgtggccaacggcgacagcgaggtggtgctgaagaagctgaagaagagcctgaacgtggccaagagcgagttcgacagggacgccgccatgcagaggaagctggagaagatggccgaccaggccatgacccagatgtacaagcaggccaggagcgaggacaagagggccaaggtgaccagcgccatgcagaccatgctgttcaccatgctgaggaagctggacaacgacgccctgaacaacatcatcaacaacgccagggacggctgcgtgcccctgaacatcatccccctgaccaccgccgccaagctgatggtggtgatccccgactacaacacctacaagaacacctgcgacggcaccaccttcacctacgccagcgccctgtgggagatccagcaggtggtggacgccgacagcaagatcgtgcagctgagcgagatcagcatggacaacagccccaacctggcctggcccctgatcgtgaccgccctgagggccaacagcgccgtgaagctgcagtgatga

SEQ ID NO: 80 - DNA, 6582 bp, synthetic construct
  source 1..6582: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctaacaacgagctgagccccgtggccctgaggcagatgagctgcgccgccggcaccacccagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacctgtacttcatcaagggcctgaacaacctgaacaggggcatggtgctgggcagcctggccgccaccgtgaggctgcagggaagcggagagggcaggggaagtcttctaacatgcggggacgtggaggaaaatcccggccccgccggcaacgccaccgaggtgcccgccaacagcaccgtgctgagcttctgcgccttcgccgtggacgccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaagaacaccgtgtgcaccgtgtgcggcatgtggaagggctacggctgcagctgcgaccagctgagggagcccatgctgcagggaagcggacagtgtactaattatgctctcttgaaattggctggagatgttgagagcaacccaggtcccgccgtgggcgcctgcgtgctgtgcaacagccagaccagcctgaggtgcggcgcctgcatcaggaggcccttcctgtgctgcaagtgctgctacgaccacgtgatcagcaccagccacaagctggtgctgagcgtgaacccctacgtgtgcaacgcccccggctgcgacgtgaccgacgtgacccagctgtacctgggcggcatgagctactactgcaagagccacaagccccccatcagcttccccctgtgcgccaacggccaggtgttcggcctgtacaagaacacctgcgtgggcagcgacaacgtgaccgacttcaacgccatcgccacctgcgactggaccaacgccggcgactacatcctggccaacacctgcaccgagaggctgaagctgttcgccgccgagaccctgaaggccaccgaggagaccttcaagctgagctacggcatcgccaccgtgagggaggtgctgagcgacagggagctgcacctgagctgggaggtgggcaagcccaggccccccctgaacaggaactacgtgttcaccggctacagggtgaccaagaacagcaaggtgcagatcggcgagtacaccttcgagaagggcgactacggcgacgccgtggtgtacaggggcaccaccacctacaagctgaacgtgggcgactacttcgtgctgaccagccacaccgtgatgcccctgagcgcccccaccctggtgccccaggagcactacgtgaggatcaccggcctgtaccccaccctgaacatcagcgacgagttcagcagcaacgtggccaactaccagaaggtgggcatgcagaagtacagcaccctgcagggcccccccggcaccggcaagagccacttcgccatcggcctggccctgtactaccccagcgccaggatcgtgtacaccgcctgcagccacgccgccgtggacgccctgtgcgagaaggccctgaagtacctgcccatcgacaagtgcagcaggatcatccccgccagggccagggtggagtgcttcgacaagttcaaggtgaacagcaccctggagcagtacgtgttctgcaccgtgaacgccctgcccgagaccaccgccgacatcgtggtgttcgacgagatcagcatggccaccaactacgacctgagcgtggtgaacgccaggctgagggccaagcactacgtgtacatcggcgaccccgcccagctgcccgcccccaggaccctgctgaccaagggcaccctggagcccgagtacttcaacagcgtgtgcaggctgatgaagaccatcggccccgacatgttcctgggcacctgcaggaggtgccccgccgagatcgtggacaccgtgagcgccctggtgtacgacaacaagctgaaggcccacaaggacaagagcgcccagtgcttcaagatgttctacaagggcgtgatcacccacgacgtgagcagcgccatcaacaggccccagatcggcgtggtgagggagttcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcagtgatga

SEQ ID NO: 81 - DNA, 6582 bp, synthetic construct
  source 1..6582: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctaacaacgagctgagccccgtggccctgaggcagatgagctgcgccgccggcaccacccagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacctgtacttcatcaagggcctgaacaacctgaacaggggcatggtgctgggcagcctggccgccaccgtgaggctgcagggaagcggagagggcaggggaagtcttctaacatgcggggacgtggaggaaaatcccggccccgccggcaacgccaccgaggtgcccgccaacagcaccgtgctgagcttctgcgccttcgccgtggacgccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaagaacaccgtgtgcaccgtgtgcggcatgtggaagggctacggctgcagctgcgaccagctgagggagcccatgctgcagggaagcggacagtgtactaattatgctctcttgaaattggctggagatgttgagagcaacccaggtcccgccgtgggcgcctgcgtgctgtgcaacagccagaccagcctgaggtgcggcgcctgcatcaggaggcccttcctgtgctgcaagtgctgctacgaccacgtgatcagcaccagccacaagctggtgctgagcgtgaacccctacgtgtgcaacgcccccggctgcgacgtgaccgacgtgacccagctgtacctgggcggcatgagctactactgcaagagccacaagccccccatcagcttccccctgtgcgccaacggccaggtgttcggcctgtacaagaacacctgcgtgggcagcgacaacgtgaccgacttcaacgccatcgccacctgcgactggaccaacgccggcgactacatcctggccaacacctgcaccgagaggctgaagctgttcgccgccgagaccctgaaggccaccgaggagaccttcaagctgagctacggcatcgccaccgtgagggaggtgctgagcgacagggagctgcacctgagctgggaggtgggcaagcccaggccccccctgaacaggaactacgtgttcaccggctacagggtgaccaagaacagcaaggtgcagatcggcgagtacaccttcgagaagggcgactacggcgacgccgtggtgtacaggggcaccaccacctacaagctgaacgtgggcgactacttcgtgctgaccagccacaccgtgatgcccctgagcgcccccaccctggtgccccaggagcactacgtgaggatcaccggcctgtaccccaccctgaacatcagcgacgagttcagcagcaacgtggccaactaccagaaggtgggcatgcagaagtacagcaccctgcagggcccccccggcaccggcaagagccacttcgccatcggcctggccctgtactaccccagcgccaggatcgtgtacaccgcctgcagccacgccgccgtggacgccctgtgcgagaaggccctgaagtacctgcccatcgacaagtgcagcaggatcatccccgccagggccagggtggagtgcttcgacaagttcaaggtgaacagcaccctggagcagtacgtgttctgcaccgtgaacgccctgcccgagaccaccgccgacatcgtggtgttcgacgagatcagcatggccaccaactacgacctgagcgtggtgaacgccaggctgagggccaagcactacgtgtacatcggcgaccccgcccagctgcccgcccccaggaccctgctgaccaagggcaccctggagcccgagtacttcaacagcgtgtgcaggctgatgaagaccatcggccccgacatgttcctgggcacctgcaggaggtgccccgccgagatcgtggacaccgtgagcgccctggtgtacgacaacaagctgaaggcccacaaggacaagagcgcccagtgcttcaagatgttctacaagggcgtgatcacccacgacgtgagcagcgccatcaacaggccccagatcggcgtggtgagggagttcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcagtgatga

SEQ ID NO: 82 - DNA, 6573 bp, synthetic construct
  source 1..6573: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagggaagcggagagggcaggggaagtcttctaacatgcggggacgtggaggaaaatcccggccccagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagtgatga

SEQ ID NO: 83 - DNA, 6573 bp, synthetic construct
  source 1..6573: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagggaagcggagagggcaggggaagtcttctaacatgcggggacgtggaggaaaatcccggccccagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagtgatga

SEQ ID NO: 84 - DNA, 4785 bp, synthetic construct
  source 1..4785: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagctgaggggcaccgccgtgatgagcctgaaggagggccagatcaacgacatgatcctgagcctgctgagcaagggcaggctgatcatcagggagaacaacagggtggtgatcagcagcgacgtgctggtgaacaactgatga

SEQ ID NO: 85 - DNA, 4785 bp, synthetic construct
  source 1..4785: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagctgaggggcaccgccgtgatgagcctgaaggagggccagatcaacgacatgatcctgagcctgctgagcaagggcaggctgatcatcagggagaacaacagggtggtgatcagcagcgacgtgctggtgaacaactgatga

SEQ ID NO: 86 - DNA, 4716 bp, synthetic construct
  source 1..4716: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggacctgttcatgaggatcttcaccatcggcaccgtgaccctgaagcagggcgagatcaaggacgccacccccagcgacttcgtgagggccaccgccaccatccccatccaggccagcctgcccttcggctggctgatcgtgggcgtggccctgctggccgtgttccagagcgccagcaagatcatcaccctgaagaagaggtggcagctggccctgagcaagggcgtgcacttcgtgtgcaacctgctgctgctgttcgtgaccgtgtacagccacctgctgctggtggccgccggcctggaggcccccttcctgtacctgtacgccctggtgtacttcctgcagagcatcaacttcgtgaggatcatcatgaggctgtggctgtgctggaagtgcaggagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgtgatga

SEQ ID NO: 87 - DNA, 4716 bp, synthetic construct
  source 1..4716: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatggacctgttcatgaggatcttcaccatcggcaccgtgaccctgaagcagggcgagatcaaggacgccacccccagcgacttcgtgagggccaccgccaccatccccatccaggccagcctgcccttcggctggctgatcgtgggcgtggccctgctggccgtgttccagagcgccagcaagatcatcaccctgaagaagaggtggcagctggccctgagcaagggcgtgcacttcgtgtgcaacctgctgctgctgttcgtgaccgtgtacagccacctgctgctggtggccgccggcctggaggcccccttcctgtacctgtacgccctggtgtacttcctgcagagcatcaacttcgtgaggatcatcatgaggctgtggctgtgctggaagtgcaggagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgtgatga

SEQ ID NO: 88 - DNA, 5915 bp, synthetic construct
  source 1..5915: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 89 - DNA, 5915 bp, synthetic construct
  source 1..5915: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 90 - DNA, 4771 bp, synthetic construct
  source 1..4771: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 91 - DNA, 4771 bp, synthetic construct
  source 1..4771: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 92 - DNA, 2209 bp, synthetic construct
  source 1..2209: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgagcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 93 - DNA, 4639 bp, synthetic construct
  source 1..4639: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatggaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcaggccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagcttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 94 - DNA, 1177 bp, synthetic construct
  source 1..1177: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgtacagcttcgtgagcgaggagaccggcaccctgatcgtgaacagcgtgctgctgttcctggccttcgtggtgttcctgctggtgaccctggccatcctgaccgccctgaggctgtgcgcctactgctgcaacatcgtgaacgtgagcctggtgaagcccagcttctacgtgtacagcagggtgaagaacctgaacagcagcagggtgcccgacctgctggtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 95 - DNA, 1618 bp, synthetic construct
  source 1..1618: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatggccgacagcaacggcaccatcaccgtggaggagctgaagaagctgctggagcagtggaacctggtgatcggcttcctgttcctgacctggatctgcctgctgcagttcgcctacgccaacaggaacaggttcctgtacatcatcaagctgatcttcctgtggctgctgtggcccgtgaccctggcctgcttcgtgctggccgccgtgtacaggatcaactggatcaccggcggcatcgccatcgccatggcctgcctggtgggcctgatgtggctgagctacttcatcgccagcttcaggctgttcgccaggaccaggagcatgtggagcttcaaccccgagaccaacatcctgctgaacgtgcccctgcacggcaccatcctgaccaggcccctgctggagagcgagctggtgatcggcgccgtgatcctgaggggccacctgaggatcgccggccaccacctgggcaggtgcgacatcaaggacctgcccaaggagatcaccgtggccaccagcaggaccctgagctactacaagctgggcgccagccagagggtggccggcgacagcggcttcgccgcctacagcaggtacaggatcggcaactacaagctgaacaccgaccacagcagcagcagcgacaacatcgccctgctggtgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 96 - DNA, 1039 bp, synthetic construct
  source 1..1039: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgaagatcatcctgttcctggccctgatcaccctggccacctgcgagctgtaccactaccaggagtgcgtgaggggcaccaccgtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 97 - DNA, 3241 bp, synthetic construct
  source 1..3241: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatggcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcactgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 98 - DNA, 1873 bp, synthetic construct
  source 1..1873: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgagcggcttcaggaagatggccttccccagcggcaaggtggagggctgcatggtgcaggtgacctgcggcaccaccaccctgaacggcctgtggctggacgacgtggtgtactgccccaggcacgtgatctgcaccagcgaggacatgctgaaccccaactacgaggacctgctgatcaggaagagcaaccacaacttcctggtgcaggccggcaacgtgcagctgagggtgatcggccacagcatgcagaactgcgtgctgaagctgaaggtggacaccgccaaccccaagacccccaagtacaagttcgtgaggatccagcccggccagaccttcagcgtgctggcctgctacaacggcagccccagcggcgtgtaccagtgcgccatgaggcccaacttcaccatcaagggcagcttcctgaacggcagctgcggcagcgtgggcttcaacatcgactacgactgcgtgagcttctgctacatgcaccacatggagctgcccaccggcgtgcacgccggcaccgacctggagggcaacttctacggccccttcgtggacaggcagaccgcccaggccgccggcaccgacaccaccatcaccgtgaacgtgctggcctggctgtacgccgccgtgatcaacggcgacaggtggttcctgaacaggttcaccaccaccctgaacgacttcaacctggtggccatgaagtacaactacgagcccctgacccaggaccacgtggacatcctgggccccctgagcgcccagaccggcatcgccgtgctggacatgtgcgccagcctgaaggagctgctgcagaacggcatgaacggcaggaccatcctgggcagcgccctgctggaggacgagttcacccccttcgacgtggtgaggcagtgcagcggcgtgaccttccagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 99 - DNA, 3751 bp, synthetic construct
  source 1..3751: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgagcgccgacgcccagagcttcctgaacagggtgtgcggcgtgagcgccgccaggctgaccccctgcggcaccggcaccagcaccgacgtggtgtacagggccttcgacatctacaacgacaaggtggccggcttcgccaagttcctgaagaccaactgctgcaggttccaggagaaggacgaggacgacaacctgatcgacagctacttcgtggtgaagaggcacaccttcagcaactaccagcacgaggagaccatctacaacctgctgaaggactgccccgccgtggccaagcacgacttcttcaagttcaggatcgacggcgacatggtgccccacatcagcaggcagaggctgaccaagtacaccatggccgacctggtgtacgccctgaggcacttcgacgagggcaactgcgacaccctgaaggagatcctggtgacctacaactgctgcgacgacgactacttcaacaagaaggactggtacgacttcgtggagaaccccgacatcctgagggtgtacgccaacctgggcgagagggtgaggcaggccctgctgaagaccgtgcagttctgcgacgccatgaggaacgccggcatcgtgggcgtgctgaccctggacaaccaggacctgaacggcaactggtacgacttcggcgacttcatccagaccacccccggcagcggcgtgcccgtggtggacagctactacagcctgctgatgcccatcctgaccctgaccagggccctgaccgccgagagccacgtggacaccgacctgaccaagccctacatcaagtgggacctgctgaagtacgacttcaccgaggagaggctgaagctgttcgacaggtacttcaagtactgggaccagacctaccaccccaactgcgtgaactgcctggacgacaggtgcatcctgcactgcgccaacttcaacgtgctgttcagcaccgtgttcccccccaccagcttcggccccctggtgaggaagatcttcgtggacggcgtgcccttcgtggtgagcaccggctaccacttcagggagctgggcgtggtgcacaaccaggacgtgaacctgcacagcagcaggctgagcttcaaggagctgctggtgtacgccgccgaccccgccatgcacgccgccagcggcaacctgctgctggacaagaggaccacctgcttcagcgtggccgccctgaccaacaacgtggccttccagaccgtgaagcccggcaacttcaacaaggacttctacgacttcgccgtgagcaagggcttcttcaaggagggcagcagcgtggagctgaagcacttcttcttcgcccaggacggcaacgccgccatcagcgactacgactactacaggtacaacctgcccaccatgtgcgacatcaggcagctgctgttcgtggtggaggtggtggacaagtacttcgactgctacgacggcggctgcatcaacgccaaccaggtgatcgtgaacaacctggacaagagcgccggcttccccttcaacaagtggggcaaggccaggctgtactacgacagcatgagctacgaggaccaggacgccctgttcgcctacaccaagaggaacgtgatccccaccatcacccagatgaacctgaagtacgccatcagcgccaagaacagggccaggaccgtggccggcgtgagcatctgcagcaccatgaccaacaggcagttccaccagaagctgctgaagagcatcgccgccaccaggggcgccaccgtggtgatcggcaccagcaagttctacggcggctggcacaacatgctgaagaccgtgtacagcgacgtggagaacccccacctgatgggctgggactaccccaagtgcgacagggccatgcccaacatgctgaggatcatggccagcctggtgctggccaggaagcacaccacctgctgcagcctgagccacaggttctacaggctggccaacgagtgcgcccaggtgctgagcgagatggtgatgtgcggcggcagcctgtacgtgaagcccggcggcaccagcagcggcgacgccaccaccgcctacgccaacagcgtgttcaacatctgccaggccgtgaccgccaacgtgaacgccctgctgagcaccgacggcaacaagatcgccgacaagtacgtgaggaacctgcagcacaggctgtacgagtgcctgtacaggaacagggacgtggacaccgacttcgtgaacgagttctacgcctacctgaggaagcacttcagcatgatgatcctgagcgacgacgccgtggtgtgcttcaacagcacctacgccagccagggcctggtggccagcatcaagaacttcaagagcgtgctgtactaccagaacaacgtgttcatgagcgaggccaagtgctggaccgagaccgacctgaccaagggcccccacgagttctgcagccagcacaccatgctggtgaagcagggcgacgactacgtgtacctgccctaccccgaccccagcaggatcctgggcgccggctgcttcgtggacgacatcgtgaagaccgacggcaccctgatgatcgagaggttcgtgagcctggccatcgacgcctaccccctgaccaagcaccccaaccaggagtacgccgacgtgttccacctgtacctgcagtacatcaggaagctgcacgacgagctgaccggccacatgctggacatgtacagcgtgatgctgaccaacgacaacaccagcaggtactgggagcccgagttctacgaggccatgtacaccccccacaccgtgctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 100 - DNA, 3353 bp, synthetic construct
  source 1..3353: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgagcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 101 - DNA, 5783 bp, synthetic construct
  source 1..5783: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatggaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcaggccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagcttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 102 - DNA, 2321 bp, synthetic construct
  source 1..2321: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgtacagcttcgtgagcgaggagaccggcaccctgatcgtgaacagcgtgctgctgttcctggccttcgtggtgttcctgctggtgaccctggccatcctgaccgccctgaggctgtgcgcctactgctgcaacatcgtgaacgtgagcctggtgaagcccagcttctacgtgtacagcagggtgaagaacctgaacagcagcagggtgcccgacctgctggtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 103 - DNA, 2762 bp, synthetic construct
  source 1..2762: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatggccgacagcaacggcaccatcaccgtggaggagctgaagaagctgctggagcagtggaacctggtgatcggcttcctgttcctgacctggatctgcctgctgcagttcgcctacgccaacaggaacaggttcctgtacatcatcaagctgatcttcctgtggctgctgtggcccgtgaccctggcctgcttcgtgctggccgccgtgtacaggatcaactggatcaccggcggcatcgccatcgccatggcctgcctggtgggcctgatgtggctgagctacttcatcgccagcttcaggctgttcgccaggaccaggagcatgtggagcttcaaccccgagaccaacatcctgctgaacgtgcccctgcacggcaccatcctgaccaggcccctgctggagagcgagctggtgatcggcgccgtgatcctgaggggccacctgaggatcgccggccaccacctgggcaggtgcgacatcaaggacctgcccaaggagatcaccgtggccaccagcaggaccctgagctactacaagctgggcgccagccagagggtggccggcgacagcggcttcgccgcctacagcaggtacaggatcggcaactacaagctgaacaccgaccacagcagcagcagcgacaacatcgccctgctggtgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 104 - DNA, 2183 bp, synthetic construct
  source 1..2183: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgaagatcatcctgttcctggccctgatcaccctggccacctgcgagctgtaccactaccaggagtgcgtgaggggcaccaccgtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 105 - DNA, 4385 bp, synthetic construct
  source 1..4385: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatggcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcactgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 106 - DNA, 3017 bp, synthetic construct
  source 1..3017: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgagcggcttcaggaagatggccttccccagcggcaaggtggagggctgcatggtgcaggtgacctgcggcaccaccaccctgaacggcctgtggctggacgacgtggtgtactgccccaggcacgtgatctgcaccagcgaggacatgctgaaccccaactacgaggacctgctgatcaggaagagcaaccacaacttcctggtgcaggccggcaacgtgcagctgagggtgatcggccacagcatgcagaactgcgtgctgaagctgaaggtggacaccgccaaccccaagacccccaagtacaagttcgtgaggatccagcccggccagaccttcagcgtgctggcctgctacaacggcagccccagcggcgtgtaccagtgcgccatgaggcccaacttcaccatcaagggcagcttcctgaacggcagctgcggcagcgtgggcttcaacatcgactacgactgcgtgagcttctgctacatgcaccacatggagctgcccaccggcgtgcacgccggcaccgacctggagggcaacttctacggccccttcgtggacaggcagaccgcccaggccgccggcaccgacaccaccatcaccgtgaacgtgctggcctggctgtacgccgccgtgatcaacggcgacaggtggttcctgaacaggttcaccaccaccctgaacgacttcaacctggtggccatgaagtacaactacgagcccctgacccaggaccacgtggacatcctgggccccctgagcgcccagaccggcatcgccgtgctggacatgtgcgccagcctgaaggagctgctgcagaacggcatgaacggcaggaccatcctgggcagcgccctgctggaggacgagttcacccccttcgacgtggtgaggcagtgcagcggcgtgaccttccagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 107 - DNA, 4895 bp, synthetic construct
  source 1..4895: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgagcgccgacgcccagagcttcctgaacagggtgtgcggcgtgagcgccgccaggctgaccccctgcggcaccggcaccagcaccgacgtggtgtacagggccttcgacatctacaacgacaaggtggccggcttcgccaagttcctgaagaccaactgctgcaggttccaggagaaggacgaggacgacaacctgatcgacagctacttcgtggtgaagaggcacaccttcagcaactaccagcacgaggagaccatctacaacctgctgaaggactgccccgccgtggccaagcacgacttcttcaagttcaggatcgacggcgacatggtgccccacatcagcaggcagaggctgaccaagtacaccatggccgacctggtgtacgccctgaggcacttcgacgagggcaactgcgacaccctgaaggagatcctggtgacctacaactgctgcgacgacgactacttcaacaagaaggactggtacgacttcgtggagaaccccgacatcctgagggtgtacgccaacctgggcgagagggtgaggcaggccctgctgaagaccgtgcagttctgcgacgccatgaggaacgccggcatcgtgggcgtgctgaccctggacaaccaggacctgaacggcaactggtacgacttcggcgacttcatccagaccacccccggcagcggcgtgcccgtggtggacagctactacagcctgctgatgcccatcctgaccctgaccagggccctgaccgccgagagccacgtggacaccgacctgaccaagccctacatcaagtgggacctgctgaagtacgacttcaccgaggagaggctgaagctgttcgacaggtacttcaagtactgggaccagacctaccaccccaactgcgtgaactgcctggacgacaggtgcatcctgcactgcgccaacttcaacgtgctgttcagcaccgtgttcccccccaccagcttcggccccctggtgaggaagatcttcgtggacggcgtgcccttcgtggtgagcaccggctaccacttcagggagctgggcgtggtgcacaaccaggacgtgaacctgcacagcagcaggctgagcttcaaggagctgctggtgtacgccgccgaccccgccatgcacgccgccagcggcaacctgctgctggacaagaggaccacctgcttcagcgtggccgccctgaccaacaacgtggccttccagaccgtgaagcccggcaacttcaacaaggacttctacgacttcgccgtgagcaagggcttcttcaaggagggcagcagcgtggagctgaagcacttcttcttcgcccaggacggcaacgccgccatcagcgactacgactactacaggtacaacctgcccaccatgtgcgacatcaggcagctgctgttcgtggtggaggtggtggacaagtacttcgactgctacgacggcggctgcatcaacgccaaccaggtgatcgtgaacaacctggacaagagcgccggcttccccttcaacaagtggggcaaggccaggctgtactacgacagcatgagctacgaggaccaggacgccctgttcgcctacaccaagaggaacgtgatccccaccatcacccagatgaacctgaagtacgccatcagcgccaagaacagggccaggaccgtggccggcgtgagcatctgcagcaccatgaccaacaggcagttccaccagaagctgctgaagagcatcgccgccaccaggggcgccaccgtggtgatcggcaccagcaagttctacggcggctggcacaacatgctgaagaccgtgtacagcgacgtggagaacccccacctgatgggctgggactaccccaagtgcgacagggccatgcccaacatgctgaggatcatggccagcctggtgctggccaggaagcacaccacctgctgcagcctgagccacaggttctacaggctggccaacgagtgcgcccaggtgctgagcgagatggtgatgtgcggcggcagcctgtacgtgaagcccggcggcaccagcagcggcgacgccaccaccgcctacgccaacagcgtgttcaacatctgccaggccgtgaccgccaacgtgaacgccctgctgagcaccgacggcaacaagatcgccgacaagtacgtgaggaacctgcagcacaggctgtacgagtgcctgtacaggaacagggacgtggacaccgacttcgtgaacgagttctacgcctacctgaggaagcacttcagcatgatgatcctgagcgacgacgccgtggtgtgcttcaacagcacctacgccagccagggcctggtggccagcatcaagaacttcaagagcgtgctgtactaccagaacaacgtgttcatgagcgaggccaagtgctggaccgagaccgacctgaccaagggcccccacgagttctgcagccagcacaccatgctggtgaagcagggcgacgactacgtgtacctgccctaccccgaccccagcaggatcctgggcgccggctgcttcgtggacgacatcgtgaagaccgacggcaccctgatgatcgagaggttcgtgagcctggccatcgacgcctaccccctgaccaagcaccccaaccaggagtacgccgacgtgttccacctgtacctgcagtacatcaggaagctgcacgacgagctgaccggccacatgctggacatgtacagcgtgatgctgaccaacgacaacaccagcaggtactgggagcccgagttctacgaggccatgtacaccccccacaccgtgctgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 108 - DNA, 5148 bp, synthetic construct
  source 1..5148: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgagcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcctgatga

SEQ ID NO: 109 - DNA, 3825 bp, synthetic construct
  source 1..3825: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgatga

SEQ ID NO: 110 - DNA, 7575 bp, synthetic construct
  source 1..7575: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgaccaggaaccccgcctggaggaaggccgtgttcatcagcccctacaacagccagaacgccgtggccagcaagatcctgggcctgcccacccagaccgtggacagcagccagggcagcgagtacgactacgtgatcttcacccagaccaccgagaccgcccacagctgcaacgtgaacaggttcaacgtggccatcaccagggccaaggtgggcatcctgtgcatcatgagcgacagggacctgtacgacaagctgcagttcaccagcctggagatccccaggaggaacgtggccaccctgcaggccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcagagcctggagaacgtggccttcaacgtggtgaacaagggccacttcgacggccagcagggcgaggtgcccgtgagcatcatcaacaacaccgtgtacaccaaggtggacggcgtggacgtggagctgttcgagaacaagaccaccctgcccgtgaacgtggccttcgagctgtgggccaagaggaacatcaagcccgtgcccgaggtgaagatcctgaacaacctgggcgtggacatcgccgccaacaccgtgatctgggactacaagagggacgcccccgcccacatcagcaccatcggcgtgtgcagcatgaccgacatcgccaagaagcccaccgagaccatctgcgcccccctgaccgtgttcttcgacggcagggtggacggccaggtggacctgttcaggaacgccaggaacggcgtgctgatcaccgagggcagcgtgaagggcctgcagcccagcgtgggccccaagcaggccagcctgaacggcgtgaccctgatcggcgaggccgtgaagacccagttcaactactacaagaaggtggacggcgtggtgcagcagctgcccgagacctacttcacccagagcaggaacctgcaggagttcaagcccaggagccagatggagatcgacttcctggagctggccatggacgagttcatcgagaggtacaagctggagggctacgccttcgagcacatcgtgtacggcgacttcagccacagccagctgggcggcctgcacctgctgatcggcctggccaagaggttcaaggagagccccttcgagctggaggacttcatccccatggacagcaccgtgaagaactacttcatcaccgacgcccagaccggcagcagcaagtgcgtgtgcagcgtgatcgacctgctgctggacgacttcgtggagatcatcaagagccaggacctgagcgtggtgagcaaggtggtgaaggtgaccatcgactacaccgagatcagcttcatgctgtggtgcaaggacggccacgtggagaccttctaccccaagctgcagagcagccaggcctggcagcccggcgtggccatgcccaacctgtacaagatgcagaggatgctgctggagaagtgcgacctgcagaactacggcgacagcgccaccctgcccaagggcatcatgatgaacgtggccaagtacacccagctgtgccagtacctgaacaccctgaccctggccgtgccctacaacatgagggtgatccacttcggcgccggcagcgacaagggcgtggcccccggcaccgccgtgctgaggcagtggctgcccaccggcaccctgctggtggacagcgacctgaacgacttcgtgagcgacgccgacagcaccctgatcggcgactgcgccaccgtgcacaccgccaacaagtgggacctgatcatcagcgacatgtacgaccccaagaccaagaacgtgaccaaggagaacgacagcaaggagggcttcttcacctacatctgcggcttcatccagcagaagctggccctgggcggcagcgtggccatcaagatcaccgagcacagctggaacgccgacctgtacaagctgatgggccacttcgcctggtggaccgccttcgtgaccaacgtgaacgccagcagcagcgaggccttcctgatcggctgcaactacctgggcaagcccagggagcagatcgacggctacgtgatgcacgccaactacatcttctggaggaacaccaaccccatccagctgagcagctacagcctgttcgacatgagcaagttccccctgaagcttgatga

SEQ ID NO: 111 - DNA, 5001 bp, synthetic construct
  source 1..5001: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgtacagcttcgtgagcgaggagaccggcaccctgatcgtgaacagcgtgctgctgttcctggccttcgtggtgttcctgctggtgaccctggccatcctgaccgccctgaggctgtgcgcctactgctgcaacatcgtgaacgtgagcctggtgaagcccagcttctacgtgtacagcagggtgaagaacctgaacagcagcagggtgcccgacctgctggtgggaagcggagccacgaacttctctctgttaaagcaagcaggagatgttgaagaaaaccccgggcctatggccgacagcaacggcaccatcaccgtggaggagctgaagaagctgctggagcagtggaacctggtgatcggcttcctgttcctgacctggatctgcctgctgcagttcgcctacgccaacaggaacaggttcctgtacatcatcaagctgatcttcctgtggctgctgtggcccgtgaccctggcctgcttcgtgctggccgccgtgtacaggatcaactggatcaccggcggcatcgccatcgccatggcctgcctggtgggcctgatgtggctgagctacttcatcgccagcttcaggctgttcgccaggaccaggagcatgtggagcttcaaccccgagaccaacatcctgctgaacgtgcccctgcacggcaccatcctgaccaggcccctgctggagagcgagctggtgatcggcgccgtgatcctgaggggccacctgaggatcgccggccaccacctgggcaggtgcgacatcaaggacctgcccaaggagatcaccgtggccaccagcaggaccctgagctactacaagctgggcgccagccagagggtggccggcgacagcggcttcgccgcctacagcaggtacaggatcggcaactacaagctgaacaccgaccacagcagcagcagcgacaacatcgccctgctggtgcagggaagcggagccacgaacttctctctgttaaagcaagcaggagatgttgaagaaaaccccgggcctatgaagatcatcctgttcctggccctgatcaccctggccacctgcgagctgtaccactaccaggagtgcgtgaggggcaccaccgtgtgatga

SEQ ID NO: 112 - DNA, 10023 bp, synthetic construct
  source 1..10023: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcacggaagcggagccacgaacttctctctgttaaagcaagcaggagatgttgaagaaaaccccgggcctagcggcttcaggaagatggccttccccagcggcaaggtggagggctgcatggtgcaggtgacctgcggcaccaccaccctgaacggcctgtggctggacgacgtggtgtactgccccaggcacgtgatctgcaccagcgaggacatgctgaaccccaactacgaggacctgctgatcaggaagagcaaccacaacttcctggtgcaggccggcaacgtgcagctgagggtgatcggccacagcatgcagaactgcgtgctgaagctgaaggtggacaccgccaaccccaagacccccaagtacaagttcgtgaggatccagcccggccagaccttcagcgtgctggcctgctacaacggcagccccagcggcgtgtaccagtgcgccatgaggcccaacttcaccatcaagggcagcttcctgaacggcagctgcggcagcgtgggcttcaacatcgactacgactgcgtgagcttctgctacatgcaccacatggagctgcccaccggcgtgcacgccggcaccgacctggagggcaacttctacggccccttcgtggacaggcagaccgcccaggccgccggcaccgacaccaccatcaccgtgaacgtgctggcctggctgtacgccgccgtgatcaacggcgacaggtggttcctgaacaggttcaccaccaccctgaacgacttcaacctggtggccatgaagtacaactacgagcccctgacccaggaccacgtggacatcctgggccccctgagcgcccagaccggcatcgccgtgctggacatgtgcgccagcctgaaggagctgctgcagaacggcatgaacggcaggaccatcctgggcagcgccctgctggaggacgagttcacccccttcgacgtggtgaggcagtgcagcggcgtgaccttccagggaagcggagccacgaacttctctctgttaaagcaagcaggagatgttgaagaaaaccccgggcctagcgccgacgcccagagcttcctgaacagggtgtgcggcgtgagcgccgccaggctgaccccctgcggcaccggcaccagcaccgacgtggtgtacagggccttcgacatctacaacgacaaggtggccggcttcgccaagttcctgaagaccaactgctgcaggttccaggagaaggacgaggacgacaacctgatcgacagctacttcgtggtgaagaggcacaccttcagcaactaccagcacgaggagaccatctacaacctgctgaaggactgccccgccgtggccaagcacgacttcttcaagttcaggatcgacggcgacatggtgccccacatcagcaggcagaggctgaccaagtacaccatggccgacctggtgtacgccctgaggcacttcgacgagggcaactgcgacaccctgaaggagatcctggtgacctacaactgctgcgacgacgactacttcaacaagaaggactggtacgacttcgtggagaaccccgacatcctgagggtgtacgccaacctgggcgagagggtgaggcaggccctgctgaagaccgtgcagttctgcgacgccatgaggaacgccggcatcgtgggcgtgctgaccctggacaaccaggacctgaacggcaactggtacgacttcggcgacttcatccagaccacccccggcagcggcgtgcccgtggtggacagctactacagcctgctgatgcccatcctgaccctgaccagggccctgaccgccgagagccacgtggacaccgacctgaccaagccctacatcaagtgggacctgctgaagtacgacttcaccgaggagaggctgaagctgttcgacaggtacttcaagtactgggaccagacctaccaccccaactgcgtgaactgcctggacgacaggtgcatcctgcactgcgccaacttcaacgtgctgttcagcaccgtgttcccccccaccagcttcggccccctggtgaggaagatcttcgtggacggcgtgcccttcgtggtgagcaccggctaccacttcagggagctgggcgtggtgcacaaccaggacgtgaacctgcacagcagcaggctgagcttcaaggagctgctggtgtacgccgccgaccccgccatgcacgccgccagcggcaacctgctgctggacaagaggaccacctgcttcagcgtggccgccctgaccaacaacgtggccttccagaccgtgaagcccggcaacttcaacaaggacttctacgacttcgccgtgagcaagggcttcttcaaggagggcagcagcgtggagctgaagcacttcttcttcgcccaggacggcaacgccgccatcagcgactacgactactacaggtacaacctgcccaccatgtgcgacatcaggcagctgctgttcgtggtggaggtggtggacaagtacttcgactgctacgacggcggctgcatcaacgccaaccaggtgatcgtgaacaacctggacaagagcgccggcttccccttcaacaagtggggcaaggccaggctgtactacgacagcatgagctacgaggaccaggacgccctgttcgcctacaccaagaggaacgtgatccccaccatcacccagatgaacctgaagtacgccatcagcgccaagaacagggccaggaccgtggccggcgtgagcatctgcagcaccatgaccaacaggcagttccaccagaagctgctgaagagcatcgccgccaccaggggcgccaccgtggtgatcggcaccagcaagttctacggcggctggcacaacatgctgaagaccgtgtacagcgacgtggagaacccccacctgatgggctgggactaccccaagtgcgacagggccatgcccaacatgctgaggatcatggccagcctggtgctggccaggaagcacaccacctgctgcagcctgagccacaggttctacaggctggccaacgagtgcgcccaggtgctgagcgagatggtgatgtgcggcggcagcctgtacgtgaagcccggcggcaccagcagcggcgacgccaccaccgcctacgccaacagcgtgttcaacatctgccaggccgtgaccgccaacgtgaacgccctgctgagcaccgacggcaacaagatcgccgacaagtacgtgaggaacctgcagcacaggctgtacgagtgcctgtacaggaacagggacgtggacaccgacttcgtgaacgagttctacgcctacctgaggaagcacttcagcatgatgatcctgagcgacgacgccgtggtgtgcttcaacagcacctacgccagccagggcctggtggccagcatcaagaacttcaagagcgtgctgtactaccagaacaacgtgttcatgagcgaggccaagtgctggaccgagaccgacctgaccaagggcccccacgagttctgcagccagcacaccatgctggtgaagcagggcgacgactacgtgtacctgccctaccccgaccccagcaggatcctgggcgccggctgcttcgtggacgacatcgtgaagaccgacggcaccctgatgatcgagaggttcgtgagcctggccatcgacgcctaccccctgaccaagcaccccaaccaggagtacgccgacgtgttccacctgtacctgcagtacatcaggaagctgcacgacgagctgaccggccacatgctggacatgtacagcgtgatgctgaccaacgacaacaccagcaggtactgggagcccgagttctacgaggccatgtacaccccccacaccgtgctgcagtgatga

SEQ ID NO: 113 - DNA, 6177 bp, synthetic construct
  source 1..6177: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctgcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcactgatga

SEQ ID NO: 114 - DNA, 4809 bp, synthetic construct
  source 1..4809: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcggcttcaggaagatggccttccccagcggcaaggtggagggctgcatggtgcaggtgacctgcggcaccaccaccctgaacggcctgtggctggacgacgtggtgtactgccccaggcacgtgatctgcaccagcgaggacatgctgaaccccaactacgaggacctgctgatcaggaagagcaaccacaacttcctggtgcaggccggcaacgtgcagctgagggtgatcggccacagcatgcagaactgcgtgctgaagctgaaggtggacaccgccaaccccaagacccccaagtacaagttcgtgaggatccagcccggccagaccttcagcgtgctggcctgctacaacggcagccccagcggcgtgtaccagtgcgccatgaggcccaacttcaccatcaagggcagcttcctgaacggcagctgcggcagcgtgggcttcaacatcgactacgactgcgtgagcttctgctacatgcaccacatggagctgcccaccggcgtgcacgccggcaccgacctggagggcaacttctacggccccttcgtggacaggcagaccgcccaggccgccggcaccgacaccaccatcaccgtgaacgtgctggcctggctgtacgccgccgtgatcaacggcgacaggtggttcctgaacaggttcaccaccaccctgaacgacttcaacctggtggccatgaagtacaactacgagcccctgacccaggaccacgtggacatcctgggccccctgagcgcccagaccggcatcgccgtgctggacatgtgcgccagcctgaaggagctgctgcagaacggcatgaacggcaggaccatcctgggcagcgccctgctggaggacgagttcacccccttcgacgtggtgaggcagtgcagcggcgtgaccttccagtgatga

SEQ ID NO: 115 - DNA, 6687 bp, synthetic construct
  source 1..6687: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcgccgacgcccagagcttcctgaacagggtgtgcggcgtgagcgccgccaggctgaccccctgcggcaccggcaccagcaccgacgtggtgtacagggccttcgacatctacaacgacaaggtggccggcttcgccaagttcctgaagaccaactgctgcaggttccaggagaaggacgaggacgacaacctgatcgacagctacttcgtggtgaagaggcacaccttcagcaactaccagcacgaggagaccatctacaacctgctgaaggactgccccgccgtggccaagcacgacttcttcaagttcaggatcgacggcgacatggtgccccacatcagcaggcagaggctgaccaagtacaccatggccgacctggtgtacgccctgaggcacttcgacgagggcaactgcgacaccctgaaggagatcctggtgacctacaactgctgcgacgacgactacttcaacaagaaggactggtacgacttcgtggagaaccccgacatcctgagggtgtacgccaacctgggcgagagggtgaggcaggccctgctgaagaccgtgcagttctgcgacgccatgaggaacgccggcatcgtgggcgtgctgaccctggacaaccaggacctgaacggcaactggtacgacttcggcgacttcatccagaccacccccggcagcggcgtgcccgtggtggacagctactacagcctgctgatgcccatcctgaccctgaccagggccctgaccgccgagagccacgtggacaccgacctgaccaagccctacatcaagtgggacctgctgaagtacgacttcaccgaggagaggctgaagctgttcgacaggtacttcaagtactgggaccagacctaccaccccaactgcgtgaactgcctggacgacaggtgcatcctgcactgcgccaacttcaacgtgctgttcagcaccgtgttcccccccaccagcttcggccccctggtgaggaagatcttcgtggacggcgtgcccttcgtggtgagcaccggctaccacttcagggagctgggcgtggtgcacaaccaggacgtgaacctgcacagcagcaggctgagcttcaaggagctgctggtgtacgccgccgaccccgccatgcacgccgccagcggcaacctgctgctggacaagaggaccacctgcttcagcgtggccgccctgaccaacaacgtggccttccagaccgtgaagcccggcaacttcaacaaggacttctacgacttcgccgtgagcaagggcttcttcaaggagggcagcagcgtggagctgaagcacttcttcttcgcccaggacggcaacgccgccatcagcgactacgactactacaggtacaacctgcccaccatgtgcgacatcaggcagctgctgttcgtggtggaggtggtggacaagtacttcgactgctacgacggcggctgcatcaacgccaaccaggtgatcgtgaacaacctggacaagagcgccggcttccccttcaacaagtggggcaaggccaggctgtactacgacagcatgagctacgaggaccaggacgccctgttcgcctacaccaagaggaacgtgatccccaccatcacccagatgaacctgaagtacgccatcagcgccaagaacagggccaggaccgtggccggcgtgagcatctgcagcaccatgaccaacaggcagttccaccagaagctgctgaagagcatcgccgccaccaggggcgccaccgtggtgatcggcaccagcaagttctacggcggctggcacaacatgctgaagaccgtgtacagcgacgtggagaacccccacctgatgggctgggactaccccaagtgcgacagggccatgcccaacatgctgaggatcatggccagcctggtgctggccaggaagcacaccacctgctgcagcctgagccacaggttctacaggctggccaacgagtgcgcccaggtgctgagcgagatggtgatgtgcggcggcagcctgtacgtgaagcccggcggcaccagcagcggcgacgccaccaccgcctacgccaacagcgtgttcaacatctgccaggccgtgaccgccaacgtgaacgccctgctgagcaccgacggcaacaagatcgccgacaagtacgtgaggaacctgcagcacaggctgtacgagtgcctgtacaggaacagggacgtggacaccgacttcgtgaacgagttctacgcctacctgaggaagcacttcagcatgatgatcctgagcgacgacgccgtggtgtgcttcaacagcacctacgccagccagggcctggtggccagcatcaagaacttcaagagcgtgctgtactaccagaacaacgtgttcatgagcgaggccaagtgctggaccgagaccgacctgaccaagggcccccacgagttctgcagccagcacaccatgctggtgaagcagggcgacgactacgtgtacctgccctaccccgaccccagcaggatcctgggcgccggctgcttcgtggacgacatcgtgaagaccgacggcaccctgatgatcgagaggttcgtgagcctggccatcgacgcctaccccctgaccaagcaccccaaccaggagtacgccgacgtgttccacctgtacctgcagtacatcaggaagctgcacgacgagctgaccggccacatgctggacatgtacagcgtgatgctgaccaacgacaacaccagcaggtactgggagcccgagttctacgaggccatgtacaccccccacaccgtgctgcagtgatga

SEQ ID NO: 116 - DNA, 1263 bp, synthetic construct
  source 1..1263: mol_type = other DNA
atgagcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcctgatga

SEQ ID NO: 117 - protein, 24 aa, synthetic construct
CVNFTTRTQLPPAYTNSFTRGVYY

SEQ ID NO: 118 - protein, 9 aa, synthetic construct
KSYGFQPTY

SEQ ID NO: 119 - protein, 9 aa, synthetic construct
VVGNHKYRF

SEQ ID NO: 120 - protein, 9 aa, synthetic construct
YQVGNKPCK

SEQ ID NO: 121 - protein, 9 aa, synthetic construct
CVIAWNSKK

SEQ ID NO: 122 - protein, 9 aa, synthetic construct
KGAKGLNCY

SEQ ID NO: 123 - protein, 9 aa, synthetic construct
SQCVNFTTR

SEQ ID NO: 124 - protein, 9 aa, synthetic construct
NIADYNYKL

SEQ ID NO: 125 - protein, 9 aa, synthetic construct
YLPLKSYGF

SEQ ID NO: 126 - protein, 9 aa, synthetic construct
KCYGVSLNK

SEQ ID NO: 127 - protein, 9 aa, synthetic construct
IYKTPPIKY

SEQ ID NO: 128 - protein, 9 aa, synthetic construct
CVADYSFLY

SEQ ID NO: 129 - protein, 9 aa, synthetic construct
SVYAWDRRK

SEQ ID NO: 130 - protein, 9 aa, synthetic construct
RFFRKSNLK

SEQ ID NO: 131 - protein, 9 aa, synthetic construct
DISTEIYQV

SEQ ID NO: 132 - protein, 9 aa, synthetic construct
YQPHRVVVL

SEQ ID NO: 133 - protein, 9 aa, synthetic construct
FVIRGDQVK

SEQ ID NO: 134 - protein, 9 aa, synthetic construct
NATKFSSVY

SEQ ID NO: 135 - protein, 9 aa, synthetic construct
NLCPFSEIF

SEQ ID NO: 136 - protein, 9 aa, synthetic construct
ASATVCGPK

SEQ ID NO: 137 - protein, 9 aa, synthetic construct
KINNCVADY

SEQ ID NO: 138 - protein, 15 aa, synthetic construct
NCYLPLKSYGFQPTY

SEQ ID NO: 139 - protein, 15 aa, synthetic construct
GNHKYRFRFFRKSNL

SEQ ID NO: 140 - protein, 15 aa, synthetic construct
PFERDISTEIYQVGN

SEQ ID NO: 141 - protein, 15 aa, synthetic construct
KKLDSKVVGNHKYRF

SEQ ID NO: 142 - protein, 15 aa, synthetic construct
KGLNCYLPLKSYGFQ

SEQ ID NO: 143 - protein, 15 aa, synthetic construct
LVLLPLVSSQCVNFT

SEQ ID NO: 144 - protein, 15 aa, synthetic construct
RGDQVKQIAPGQTGN

SEQ ID NO: 145 - protein, 15 aa, synthetic construct
SASFSTFKCYGVSLN

SEQ ID NO: 146 - protein, 15 aa, synthetic construct
KLDSKVVGNHKYRFR

SEQ ID NO: 147 - protein, 15 aa, synthetic construct
FAQVKQIYKTPPIKY

SEQ ID NO: 148 - protein, 15 aa, synthetic construct
ADYSFLYNSASFSTF

SEQ ID NO: 149 - protein, 15 aa, synthetic construct
ATKFSSVYAWDRRKI

SEQ ID NO: 150 - protein, 15 aa, synthetic construct
PHRVVVLSFELLHAS

SEQ ID NO: 151 - protein, 15 aa, synthetic construct
FERDISTEIYQVGNK

SEQ ID NO: 152 - protein, 15 aa, synthetic construct
AKGLNCYLPLKSYGF

SEQ ID NO: 153 - protein, 15 aa, synthetic construct
SIVRFPNITNLCPFS

SEQ ID NO: 154 - protein, 15 aa, synthetic construct
NNCVADYSFLYNSAS

SEQ ID NO: 155 - protein, 15 aa, synthetic construct
KGAKGLNCYLPLKSY

SEQ ID NO: 156 - protein, 15 aa, synthetic construct
LCPFSEIFNATKFSS

SEQ ID NO: 157 - protein, 44 aa, synthetic construct
KKLDSKVVGNHKYRFRFFRKSNLKPFERDISTEIYQVGNKPCKG